(19) United States
(12) Patent Application Publication
(10) Pub. No.: US 2024/0246980 A1
(43) Pub. Date: Jul. 25, 2024

(54) MODULATORS OF MAS-RELATED G-PROTEIN RECEPTOR D AND RELATED PRODUCTS AND METHODS

(71) Applicant: Escient Pharmaceuticals, Inc., San Diego, CA (US)

(72) Inventors: Adam Yeager, San Diego, CA (US); Marion Lanier, San Diego, CA (US); Marcos Sainz, San Diego, CA (US); Brandon Selfridge, San Diego, CA (US); Liming Huang, San Diego, CA (US); Esther Martinborough, San Diego, CA (US); Marcus Boehm, San Diego, CA (US)

(21) Appl. No.: 18/496,540

(22) Filed: Oct. 27, 2023

Related U.S. Application Data
(60) Provisional application No. 63/420,470, filed on Oct. 28, 2022.

(51) Int. Cl. C07D 487/04; A61K 31/437; A61K 31/454; A61K 31/4545; A61K 31/5025; A61K 31/5377; C07D 401/04; C07D 471/04; C07D 519/00

(52) U.S. Cl. CPC C07D 487/04; A61K 31/437; A61K 31/454; A61K 31/4545; A61K 31/5025; A61K 31/5377; C07D 401/04; C07D 471/04; C07D 519/00

(57) ABSTRACT

Methods are provided for modulating MRGPRD generally, or for treating a MRGPRD dependent condition more specifically, by contacting the MRGPRD or administering to a subject in need thereof, respectively, an effective amount of a compound having the structure of Formula (I):

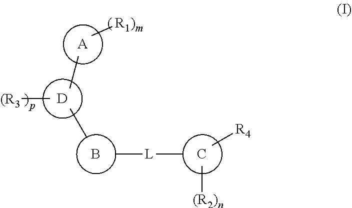

or a pharmaceutically acceptable salt, isomer, hydrate, solvate or isotope thereof, wherein A, B, C, D, L, R₁, R₂, R₃, R₄, m, n, and p are as defined herein. Pharmaceutical compositions containing such compounds, as well as to compounds themselves, are also provided.

### BACKGROUND

### Technical Field

The invention relates to modulators of the Mas-related G-protein coupled receptor D (MRGPRD), to products containing the same, as well as to methods of their use and preparation.

### Description of the Related Art

Mas-related G protein receptors (MRGPRs) are a group of orphan receptors with limited expression in very specialized tissues. Little is known about the function of most of these receptors. There are eight related receptors in this class expressed in humans, only four of which have readily identifiable orthologs in other species (i.e., MRGPR D, E, F and G). The other four receptors (MRGPR X1, X2, X3 and X4) have no counterpart, based on homology, in species other than humans and primates.

### BRIEF SUMMARY

This invention is based, in part, on the identification of MRGPRD or MRGPRD ortholog modulator compounds. MRGPRD corresponds functionally to mouse and rat Mrgprd. MRGPRD and its orthologs are expressed in the dorsal root ganglia as well as several peripheral organs. MRGPRD and its orthologs have been shown to be involved in pain signaling, physiological and pathophysiological processes of the gastrointestinal (GI) tract, Ca²⁺ dysregulation in the heart (cardiac output and vascular tone) and have also been shown to be expressed in skin, immune cells, the eye, kidney, and brain.

In one embodiment is provided a method of treating a MRGPRD or a MRGPRD ortholog dependent condition, by administering to a subject in need thereof an effective amount of the pharmaceutical composition of the modulator compounds of the present invention.

Accordingly, in some embodiments, compounds having activity as modulators of the Mas-related G-protein coupled receptor D are provided, the compounds having the following structure (I):

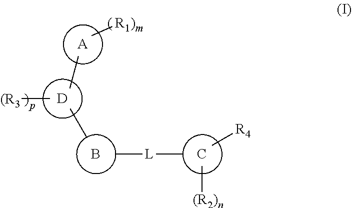

or a pharmaceutically acceptable salt, isomer, hydrate, solvate or isotope thereof, wherein A, B, C, D, L, R₁, R₂, R₃, R₄, m, n, and p are as defined below.

In other embodiments, compounds having activity as modulators of the Mas-related G-protein coupled receptor D are provided, the compounds having the following structure (II):

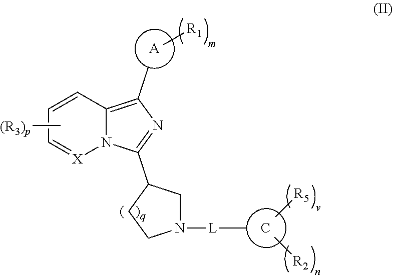

or a pharmaceutically acceptable salt, isomer, hydrate, solvate or isotope thereof, wherein A, C, L, X, R₁, R₂, R₃, R₅, m, n, p, q, and v are as defined below.

In some embodiments, compounds having activity as modulators of the Mas-related G-protein coupled receptor D are provided, the compounds having the following structure (III):

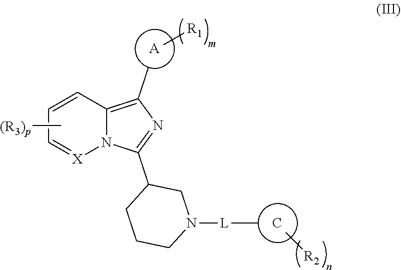

or a pharmaceutically acceptable salt, isomer, hydrate, solvate or isotope thereof, wherein A, C, L, X, R₁, R₂, R₃, m, n, and p are as defined below.

In some embodiments, compounds having activity as modulators of the Mas-related G-protein coupled receptor D are provided, the compounds having the following structure (IV):

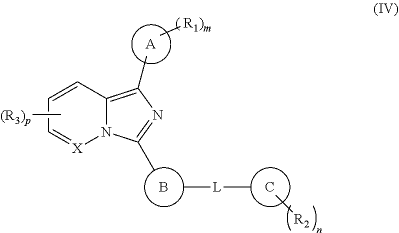

or a pharmaceutically acceptable salt, isomer, hydrate, solvate or isotope thereof, wherein A, B, C, L, X, R₁, R₂, R₃, m, n, and p are as defined below.

In some embodiments, compounds having activity as modulators of the Mas-related G-protein coupled receptor D are provided, the compounds having the following structure (V):

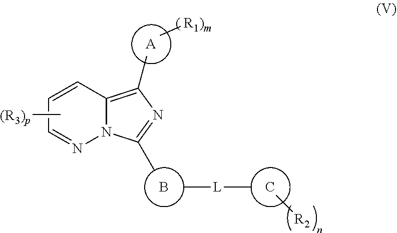

or a pharmaceutically acceptable salt, isomer, hydrate, solvate or isotope thereof, wherein A, B, C, L, R₁, R₂, R₃, m, n, and p are as defined below.

In some embodiments, compounds having activity as modulators of the Mas-related G-protein coupled receptor D are provided, the compounds having the following structure (VI):

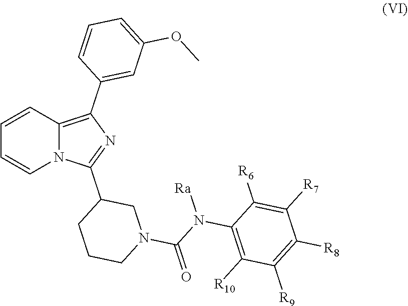

or a pharmaceutically acceptable salt, isomer, hydrate, solvate or isotope thereof, wherein Ra, R₆, R₇, R₈, R₉, and R₁₀ are as defined below.

In some embodiments, compounds having activity as modulators of the Mas-related G-protein coupled receptor D are provided, the compounds having the following structure (VII):

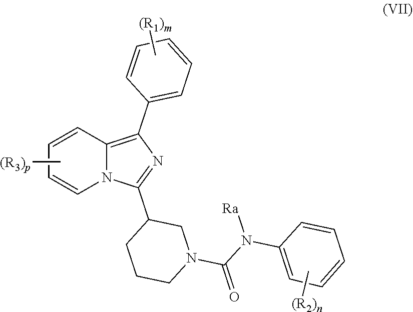

or a pharmaceutically acceptable salt, isomer, hydrate, solvate or isotope thereof, wherein Ra, R₁, R₂, R₃, m, n, and p are as defined below.

In some embodiments, compounds having activity as modulators of the Mas-related G-protein coupled receptor D are provided, the compounds having the following structure (VIII):

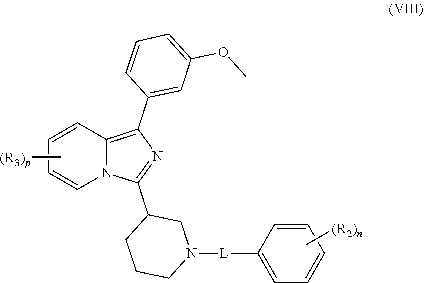

or a pharmaceutically acceptable salt, isomer, hydrate, solvate or isotope thereof, wherein L, R₂, R₃, n, and p are as defined below.

In some embodiments, compounds having activity as modulators of the Mas-related G-protein coupled receptor D are provided, the compounds having the following structure (IX):

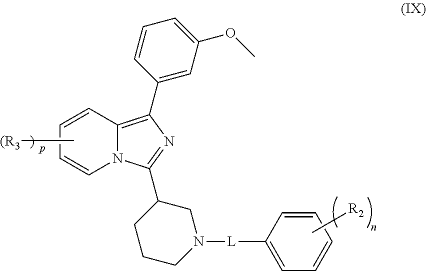

or a pharmaceutically acceptable salt, isomer, hydrate, solvate or isotope thereof, wherein L, R₂, R₃, n, and p are as defined below.

In some embodiments, compounds having activity as modulators of the Mas-related G-protein coupled receptor D are provided, the compounds having the following structure (X):

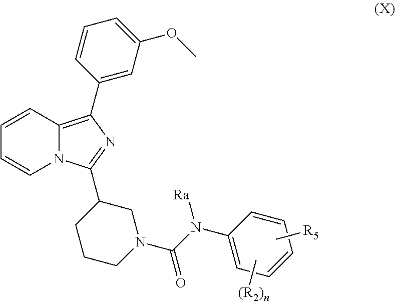

or a pharmaceutically acceptable salt, isomer, hydrate, solvate or isotope thereof, wherein Ra, R₂, R₅, and n are as defined below.

In some embodiments, compounds having activity as modulators of the Mas-related G-protein coupled receptor D are provided, the compounds having the following structure (XI):

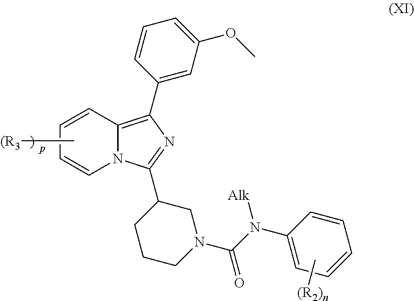

or a pharmaceutically acceptable salt, isomer, hydrate, solvate or isotope thereof, wherein Ak, R₂, R₃, n and p are as defined below.

In some embodiments, compounds having activity as modulators of the Mas-related G-protein coupled receptor D are provided, the compounds having the following structure (XII):

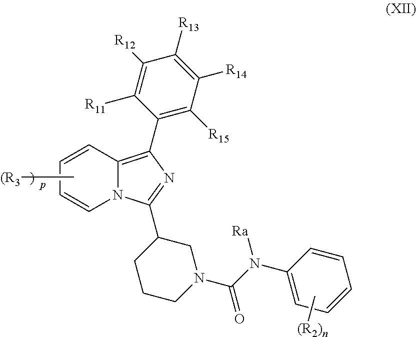

or a pharmaceutically acceptable salt, isomer, hydrate, solvate or isotope thereof, wherein Ra, R₂, R₃, R₁₁, R₁₂, R₁₃, R₁₄, R₁₅, n and p are as defined below.

In some embodiments, compounds having activity as modulators of the Mas-related G-protein coupled receptor D are provided, the compounds having the following structure (XIII):

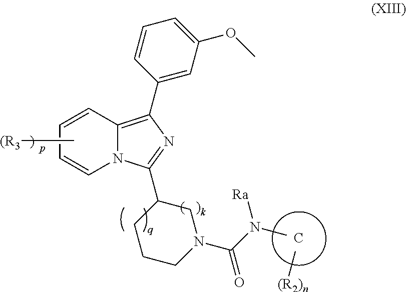

or a pharmaceutically acceptable salt, isomer, hydrate, solvate or isotope thereof, wherein C, Ra, R₂, R₃, n, p, q, and k are as defined below.

In some embodiments, compounds having activity as modulators of the Mas-related G-protein coupled receptor D are provided, the compounds having the following structure (XIV):

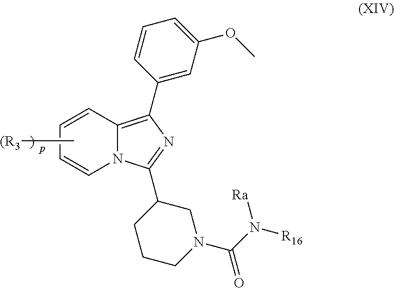

or a pharmaceutically acceptable salt, isomer, hydrate, solvate or isotope thereof, wherein Ra, R₃, R₁₆, and p are as defined below.

In additional embodiments, methods are provided for modulating a MRGPRD by contacting the MRGPRD with an effective amount of a compound having the structure of Formula (XV):

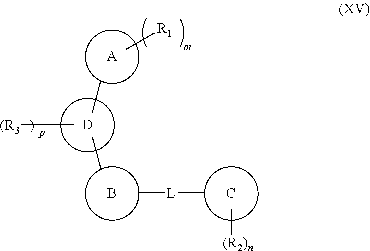

or a pharmaceutically acceptable salt, isomer, hydrate, solvate or isotope thereof, wherein A, B, C, D, L, R₁, R₂, R₃, m, n, and p are as defined below.

In other embodiments, methods are provided for modulating a MRGPRD by contacting the MRGPRD with an effective amount of a compound having the structure (XVa) or (XVb) as defined herein, or a pharmaceutically acceptable salt, isomer, hydrate, solvate or isotope thereof.

In yet other embodiments, pharmaceutical compositions are provided comprising a carrier or excipient and a compound having structure (I), or a pharmaceutically acceptable salt, isomer, hydrate, solvate or isotope thereof.

In other embodiments, pharmaceutical compositions are provided comprising a compound of structures (II), (III), (IV), (V), (VI), (VII), (VIII), (IX), (X), (XI), (XII), (XIII), or (XIV) as defined herein or a pharmaceutically acceptable salt, isomer, hydrate, solvate or isotope thereof.

In another embodiment, methods are provided for treating an MRGPRD dependent condition by administering to a subject in need thereof an effective amount of a compound or pharmaceutical composition having structure (I), (II), (III), (IV), (V), (VI), (VII), (VIII), (IX), (X), (XI), (XII), (XIII), (XIV), (XV), (XVa), or (XVb) or a pharmaceutically acceptable salt, isomer, hydrate, solvate or isotope thereof.

### DETAILED DESCRIPTION

### MRGPRs

Mas-related G-protein-coupled receptors (MRGPRs) comprise a subfamily of Class A receptors named after the first discovered member, Mas. MRGPRs were first identified on specialized sensory neurons that encode itch (pruriceptors) and pain (nociceptors). Both neuronal subtypes have their cell bodies residing in the dorsal root and trigeminal ganglia (DRG and TG). Importantly, activation of Mrgprs expressed at the surface of sensory neurons has been shown to induce both non-histaminergic itch, pain sensations and mechanical/visceral hypersensitivity.

The eight human MRGPRs comprise of MRGPRX1-4 (1,2,3 and 4) and MRGPRD-G (D, E, F, and G). Whilst MRGPRX1-4 are only expressed in humans and non-human primates, MRGPRD-G seem to be expressed in all mammals. The orthlogs of MRGPRD are readily identified despite low sequence homology. Of note, MRGPRD is encoded by a single copy MRGPRD gene with defined orthologues in rodents and humans and thus constitutes an attractive therapeutic target for pain and other indications.

Despite the sensory nature of these receptors, the function of these MRGPRs has proved elusive. Trianglulation of agonist profiles, receptor expression in tissues and loss/gain of function in animal (disease) models has proven important in recognizing the therapeutic potential of these receptors.

### MRGPRD Agonists

The first ligand described for MRGPRD of human, rat, mouse, and monkey was β-alanine. This amino acid analog is produced in the liver from uracil or from dietary carnosine by the enzyme carnosinase. Coupling to calcium-activated chloride channels via Gq proteins, phospholipase C, and inositol-3 phosphate-induced Ca²⁺ release has been described.

Alamandine, Ala1-Ang-(1-7), a peptide in the Renin-Angiotensin System (RAS), has also been described as a potent agonist of MRGPRD. Peptides seem to interact differently with the receptor than β-alanine, because β-alanine does not inhibit the vasodilatory actions of alamandine. However, D-Pro7-Ang-(1-7) was shown to be an inhibitor of alamandine binding to MRGPRD.

5-oxoETE, a lipid elevated in the gut of IBS patients, has been shown to induce calcium signaling in sensory neurons that is dependent on the presence of MRGPRD, suggesting that 5-oxoETE might signal in neurons via MRGPRD.

### MRGPRD Expression

In addition to DRG, the expression of MRGPRD transcripts has been identified in several peripheral organs including artery, heart, bladder, GI tract, eye, brain, and kidney. This suggests that MRGPRD may have a role in several peripheral indications

### MRGPRD in Neurons of the Dorsal Root Ganglia

MRGPRD is expressed at very high levels in most unmyelinated nociceptive neurons that are labeled by isolectin-B4 and expressed in DRG. Like other members in the MRGPRs family, MRGPRD has been suggested to be highly related to the sensation of pain and itch.

MRGPRD activation has been shown to mediate pain signaling, characterized by hypersensitivity to multiple stimuli that lead to painful sensation once integrated in the brain. Indeed, elevated expression of MRGPRD is observed in models of neuropathic pain.

### MRGPRD in the Gut

Expression of MRGPRD has been demonstrated in sensory neurons innervating the colon. In colon-projecting sensory neurons, 41% of TRPV1-positive neurons were also reported to express MRGPRD. Activation of MRGPRD signaling in the colon has been shown to participate in the development of pain sensation in the context of irritable bowel syndrome (IBS).

The expression of MRGPRD in the enteric neurons indicates that MRGPRD is highly associated with physiological and pathophysiological processes of the GI tract, such as bowel motility/dysmotility and intestinal inflammation.

In a recent study, an increase in the arachidonic acid metabolite 5-oxoETE was found in biopsies from patients with clinically established IBS compared to healthy subjects. 5-oxoETE has been shown to induce calcium signaling in sensory neurons. In the absence of MRGPRD, activation of sensory neurons by 5-oxoETE was significantly decreased, suggesting that 5-oxoETE might signal in neurons via MRGPRD.

### MRGPRD in Vascular Tissue

In the aorta/heart, MRGPRD is activated by its receptor agonist, alamandine, and produces the endothelial-dependent vasodilation in rat and mouse aortic rings. In hypertensive rats, alamandine treatment restored the contractile function and prevented Ca²⁺ dysregulation via activation of MrgprD in cardiomyocytes.

Alamandine via MRGPRD induces AMPK/NO signaling to counter-regulate ANGII-induced hypertrophy, highlighting the therapeutic potential of the alamandine/MrgD axis in the heart.

Blockers of the receptors of alternate RAS, such as the MRGPRD, increase splanchnic vascular resistance in cirrhotic animals, and thus drugs targeting the alternate RAS may be useful in the treatment of portal hypertension and liver fibrosis.

### MRGPRD in Immune Cells

MRGPRD has been found to be expressed in neutrophils and is thought to be involved in inflammatory reactions.

Alamandine, through MRGPRD receptors, do not affect MO macrophages but reduce the proinflammatory TNF-α, CCL2, and IL-1β transcript expression levels in LPS+IFN-γ-stimulated macrophages.

### MRGPRD in the Eye

MRGPRD is expressed in retinal neurons, retinal vasculature, Müller glial and RPE cells. MRGPRD-deficient mice do not exhibit gross changes in retinal morphology and thickness in aging. In vitro studies in human retinal cells show that alamandine attenuated increases in inflammatory cytokine gene expression and production of reactive oxygen species. These results support the notion that alamandine/MRGPRD may represent another new protective axis of RAS in the retina exerting anti-oxidative and anti-inflammatory effects.

### MRGPRD in the Kidney

Allantoin induces scratching behavior in mice and active DRG neurons; the calcium influx and the action potential were significantly reduced in DRG neurons of MRGPRD KO mice, suggesting a role for MRGPRD in chronic kidney disease (CKD).

### MRGPRD in the Brain

Studies in the mouse brain show that MRGPRD-positive cells have been identified in some forebrain areas, including cortex, hippocampus, amygdala, hypothalamus, habenular nuclei, striatum and pallidum, as well as in some mid-brain nuclei in a region-specific manner. The specific localization of MRGPRD in the reward- and limbic-related areas can hint at a role of MRGPRD in processes such as pain perception/modulation, synaptic plasticity, learning, memory, and cognition.

Additionally, alamandine induces antidepressant-like effects in low brain angiotensinogen transgenic rats. An MRGPRD receptor antagonist reversed the antidepressant-like effect induced by alamandine, suggesting a role of MRGPRD in the treatment of neuropsychiatric diseases.

### MRGPRD and Lung Cancer

MRGPRD is reported to be expressed in lung cancer and promotes tumorigenesis. Therefore, targeting MRGPRD may provide a novel therapy for lung or other cancers.

### Measurement of MRGPRD Activity

See example 26.

### MRGPRD Utilities

As used herein, the phrase “MRGPRD dependent condition” means a condition where the activation, over sensitization, or desensitization of MRGPRD by a natural or synthetic ligand initiates, mediates, sustains, or augments a pathological condition.

A method of treating a subject having a pathological condition is provided, the method comprising of the administration to the subject a pharmaceutically effective amount of a compound having structure (I) or a pharmaceutically acceptable salt, isomer, hydrate, solvate or isotope thereof, or a pharmaceutical composition thereof.

As mentioned above, the invention relates to modulators of MRGPRD, to products containing the same, as well as to methods of their use and preparation. This receptor mediates disorders including dry eye syndrome/keratoconjunctivitis sicca and related conditions, chronic itch (e.g., pruritus), inflammation disorders, autoimmunity, skin disorders, cardiovascular disease, renal disease, cognitive impairment due to neurodegenerative diseases, age-induced cognitive impairment, vascular cognitive impairment, post-stroke cognitive impairment, and psychiatric disorders.

### Definitions

As used herein, the following terms have the meaning defined below, unless the context indicates otherwise.

“Modulating” MRGPRD means that the compound interacts with the MRGPRD in a manner such that it functions as an inverse agonist to the receptor, and/or as a competitive antagonist to the receptor. In one embodiment, such modulation is partially or fully selective against other MRGPRs, such as MRGPRX1, X2, X3 and/or X4.

The term “agonism” is used herein to encompass compounds that interact in some way with a receptor and thereby function as an agonist, either by binding to the receptor at the binding site of its natural ligand or at locations other than the binding site. Thus, the phrase “MRGPRD agonism” is used herein to encompass compounds that interact in some way with MRGPRD and thereby function as an agonist, either by binding to the GPCR receptor at the binding site of its natural ligand, or at a location other than the binding site (i.e., allosteric binding).

Conversely, the term “antagonism” is used herein to encompass compounds that interact in some way with a receptor and thereby function as an antagonist, either by binding to the receptor at the binding site of its natural ligand or at locations other than the binding site. Thus, the phrase “MRGPRD antagonism” is used herein to encompass compounds that interact in some way with the MRGPRD and thereby function as an antagonist, either by binding to the GPCR at the binding site of its natural ligand, or at a location other than the binding site (i.e., allosteric binding).

A partial agonist is a compound that binds to and activates a receptor, but with reduced efficacy compared to a full agonist. In the presence of a full agonist, a partial agonist behaves as an effective competitive antagonist. An inverse agonist is a compound that binds to a receptor and induces an opposing pharmacological response to that of an agonist. An allosteric modulator is a compound that binds at a location distinct from the orthosteric site, or the site of action of the primary ligand, and exerts an indirect effect by influencing binding or efficacy of the primary ligand. Pure allostery exerts no effect on a protein in the absence of a primary ligand that either activates or deactivates a receptor.

“MRGPR” refers to one or more of the Mas-related G protein coupled receptors, which are a group of orphan receptors with limited expression in very specialized tissues (e.g., in mast cells and dorsal root ganglia) and barrier tissues. There are eight related receptors in this class expressed in humans, only 4 of which have readily identifiable orthologs in other species (i.e., MRGPRD, E, F and G). Some of the other four receptors (MRGPRX1, X2, X3 and X4) have counterparts in higher species including dogs and horses, but they do not have a single corresponding ortholog in rodents.

“MRGPRD,” also referred to as “MRGD,” or, “TGR7”, or “MAS related GPR family member D” refers to a member of the MRGPR family.

“Effective amount” refers to a quantity of a specified agent sufficient to achieve a desired effect in a subject being treated with that agent. Ideally, an effective amount of an agent is an amount sufficient to inhibit or treat the disease without causing substantial toxicity in the subject. The effective amount of an agent will be dependent on the subject being treated, the severity of the affliction, and the manner of administration of the pharmaceutical composition. Methods of determining an effective amount of the disclosed compound sufficient to achieve a desired effect in a subject will be understood by those of skill in the art in light of this disclosure.

In the following description, certain specific details are set forth in order to provide a thorough understanding of various embodiments of the invention. However, one skilled in the art will understand that the invention may be practiced without these details.

Unless the context requires otherwise, throughout the present specification and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless defined otherwise, all technical and scientific terms used herein have the same meaning as is commonly understood by one of skill in the art to which this invention belongs. As used in the specification and claims, the singular form “a”, “an” and “the” include plural references unless the context clearly dictates otherwise.

“Alkyl” means a saturated straight chain or branched alkyl group having from 1 to 8 carbon atoms, in some embodiments from 1 to 6 carbon atoms, in some embodiments from 1 to 4 carbon atoms, and in some embodiments from 1 to 3 carbon atoms. Examples of saturated straight chain alkyl groups include, but are not limited to, methyl, ethyl, n-propyl, n-butyl, n-pentyl-, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. Specific examples of alkyl groups include,

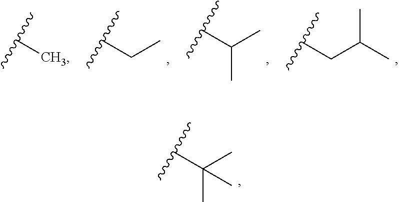

and the like.

“Alkenyl” means a straight chain or branched alkenyl group having from 2 to 8 carbon atoms, in some embodiments from 2 to 6 carbon atoms, in some embodiments from 2 to 4 carbon atoms, and in some embodiments from 2 to 3 carbon atoms. Alkenyl groups are unsaturated hydrocarbons that contain at least one carbon-carbon double bond. Examples of alkenyl groups include, but are not limited to, vinyl, propenyl, butenyl, pentenyl, and hexenyl.

“Alkynyl” means a straight chain or branched alkynyl group having from 2 to 8 carbon atoms, in some embodiments from 2 to 6 carbon atoms, in some embodiments from 2 to 4 carbon atoms, and in some embodiments from 2 to 3 carbon atoms. Alkynyl groups are unsaturated hydrocarbons that contain at least one carbon-carbon triple bond. Examples of alkynyl groups include, but are not limited to, ethynyl, propynyl, butynyl, pentynyl, and hexynyl.

“Halo” or “halogen” refers to fluorine, chlorine, bromine, and iodine.

“Hydroxy” or “hydroxyl” refers to —OH.

“Cyano” refers to —CN.

“Carboxy” or “carboxyl” refers to —CO₂H.

“Amino” refers to —NH₂.

“Aminyl” refers to —NHalkyl or N(alkyl)₂, wherein alkyl is as defined above. Examples of aminyl include, but are not limited to

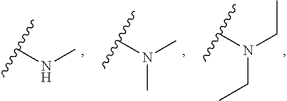

and the like.

“Aminylalkyl” refers to an aminyl as described above joined by way of an alkyl as described above (i.e., -alkyl-aminyl). Examples of aminylalkyl include, but are not limited to,

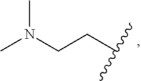 [SMILES: *CCN(C)C]

and the like.

“Cyanoalkyl” refers to CN as described above joined by way of an alkyl as described above (i.e., -alkyl-CN). Examples of cyanoalkyl include, but are not limited to,

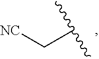 [SMILES: *CC#N]

and the like.

“Haloalkyl” refers to alkyl as defined above with one or more hydrogen atoms replaced with halogen. Examples of haloalkyl groups include, but are not limited to, —CF₃, —CHF₂, and the like.

“Hydroxylalkyl” refers to a hydroxyl as described above joined by way of an alkyl as described above (i.e., -alkyl-OH). Examples of hydroxylalkyl include, but are not limited to,

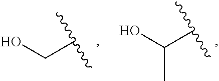

and the like.

“Alkoxy” refers to alkyl as defined above joined by way of an oxygen atom (i.e., —O-alkyl). Examples of alkoxy groups include, but are not limited to, methoxy, ethoxy, n-propoxy, n-butoxy, isopropoxy, sec-butoxy, tert-butoxy, and the like. Specific examples of alkoxy groups include,

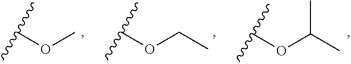

and the like.

“Alkoxyalkyl” refers to alkoxy as described above joined by way of an alkyl as described above (i.e., -alkyl-alkoxy). Examples of alkoxyalkyl groups include, but are not limited to,

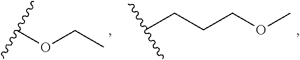

and the like.

“Alkoxyalkenyl” refers to alkoxy as described above joined by way of an alkenyl as described above (i.e., -alkenyl-alkoxy). Examples of alkoxyalkenyl groups include, but are not limited to,

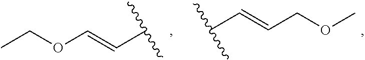

and the like.

“Haloalkoxy” refers to haloalkyl as defined above joined by way of an oxygen atom (i.e., —O-haloalkyl). Examples of haloalkoxy groups include, but are not limited to, —OCF₃, and the like.

“Alkylcarbonyl” refers to alkyl as described above joined by way of a carbonyl (i.e., —C(O)-alkyl). Examples of alkylcarbonyl groups include, but are not limited to,

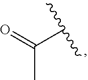 [SMILES: *C(C)=O]

and the like.

“Alkoxycarbonyl” refers to alkoxy as described above joined by way of a carbonyl (i.e., —C(O)-alkoxy). Examples of alkoxycarbonyl groups include, but are not limited to,

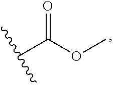 [SMILES: *C(=O)OC]

and the like.

“Aminocarbonyl” refers to amino as described above joined by way of a carbonyl (i.e., —C(O)-amino). Examples of aminocarbonyl groups include, but are not limited to,

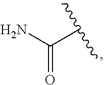 [SMILES: *C(N)=O]

and the like.

“Aminylcarbonyl” refers to aminyl as described above joined by way of a carbonyl (i.e., —C(O)-aminyl). Examples of aminylcarbonyl groups include, but are not limited to,

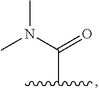 [SMILES: *C(=O)N(C)C]

and the like.

“Alkylsulfonyl” refers to alkyl as described above joined by way of a sulfonyl (i.e., —S(O)₂-alkyl). Examples of alkylsulfonyl groups include, but are not limited to, and the

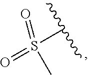 [SMILES: *S(C)(=O)=O]

like.

“Alkylsulfonylalkyl” refers to alkylsulfonyl as described above joined by way of an alkyl (i.e., -alkyl-S(O)₂-alkyl). Examples of alkylsulfonyl groups include, but are not limited to,

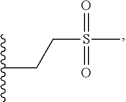 [SMILES: *CCS(C)(=O)=O]

and the like.

“Carbocycle” or “carbocyclyl” or “carbocyclic ring” refers to alkyl groups forming a ring structure, which can be substituted or unsubstituted, wherein the ring is either completely saturated, partially unsaturated, or fully unsaturated, wherein if there is unsaturation, the conjugation of the pi-electrons in the ring may give rise to aromaticity. In one embodiment, carbocycle includes cycloalkyl as defined herein. In another embodiment, carbocycle includes aryl as defined herein.

“Cycloalkyl” refers to alkyl groups forming a ring structure, which can be substituted or unsubstituted, wherein the ring is either completely saturated, partially unsaturated, or fully unsaturated, wherein if there is unsaturation, the conjugation of the pi-electrons in the ring do not give rise to aromaticity. Examples of cycloalkyl include, but are not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Specific examples of cycloalkyl groups include

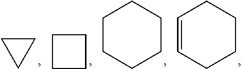

and the like.

“Cycloalkylalkyl” refers to a cycloalkyl as described above joined by way of an alkyl as described above (i.e., -alkyl-cycloalkyl). Examples of cycloalkylalkyl include, but are not limited to,

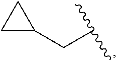 [SMILES: *CC1CC1]

and the like.

“Aryl” groups are cyclic aromatic hydrocarbons that do not contain heteroatoms. Representative aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain 6-14 carbons in the ring portions of the groups. The terms “aryl” and “aryl groups” include fused rings wherein at least one ring, but not necessarily all rings, are aromatic, such as fused aromatic-aliphatic ring systems (e.g., indanyl, tetrahydronaphthyl, and the like). In one embodiment, aryl is phenyl or naphthyl, and in another embodiment aryl is phenyl. Specific examples of aryl groups include

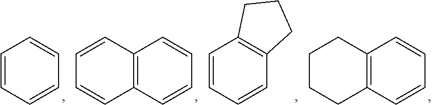

and the like.

“Arylalkyl” refers to an aryl group as described above jointed by way of an alkyl as described above (i.e., -alkyl-aryl). Examples of arylalkyl include, but are not limited to,

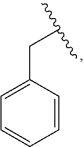 [SMILES: *Cc1ccccc1]

and the like.

“Arylsulfonyl” refers to aryl as described above joined by way of a sulfonyl (i.e., —S(O)₂-aryl). Examples of alkylsulfonyl groups include, but are not limited to,

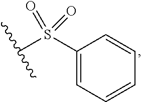 [SMILES: *S(=O)(=O)c1ccccc1]

and the like.

“Heterocycle” or “heterocyclyl” or “heterocyclic ring” refers to aromatic and non-aromatic ring moieties containing 3 or more ring members, of which one or more is a heteroatom such as, but not limited to, N, O, S, or P. In some embodiments, heterocyclyl include 3 to 20 ring members, whereas other such groups have 3 to 15 ring members, and yet other such groups have 5 to 10 ring members. In certain embodiments, “heterocycle”, “heterocyclyl”, or “heterocyclic ring” refers to a group having from 5 to 10 ring members, of which one or more is a heteroatom selected from N, O, or S. At least one ring contains a heteroatom, but every ring in a polycyclic system need not contain a heteroatom. For example, a dioxolanyl ring and a benzdioxolanyl ring system (methylenedioxyphenyl ring system) are both heterocyclyl groups within the meaning herein.

Heterocyclyl groups also include fused ring species including those having fused aromatic and non-aromatic groups. A heterocyclyl group also includes polycyclic ring systems containing a heteroatom such as, but not limited to, quinuclidyl, and also includes heterocyclyl groups that have substituents, including but not limited to alkyl, halo, amino, aminyl, hydroxy, cyano, carboxy, nitro, thio, or alkoxy groups, bonded to one of the ring members. A heterocyclyl group as defined herein can be a heteroaryl group or a partially or completely saturated cyclic group including at least one ring heteroatom. Heterocyclyl groups include, but are not limited to, pyrrolidinyl, furanyl, tetrahydrofuranyl, dioxolanyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups.

In one embodiment, heterocyclyl includes heteroaryl.

“Heterocyclylalkyl” refers to a heterocyclyl group as described above joined by way of an alkyl as described above (i.e., -alkyl-heterocyclyl).

“Heteroaryl” refers to aromatic ring moieties containing 5 or more ring members, of which, one or more is a heteroatom such as, but not limited to, N, O, and S. Heteroaryl groups include, but are not limited to, groups such as pyrrolyl, pyrazolyl, pyridinyl, pyridazinyl, pyrimidyl, pyrazyl, pyrazinyl, pyrimidinyl, thienyl, triazolyl, tetrazolyl, triazinyl, thiazolyl, thiophenyl, oxazolyl, isoxazolyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, tetrahydroisoquinolinyl, quinoxalinyl, and quinazolinyl groups. The terms “heteroaryl” and “heteroaryl groups” include fused ring compounds such as wherein at least one ring, but not necessarily all rings, are aromatic, including tetrahydroquinolinyl, tetrahydroisoquinolinyl, indolyl, and 2,3-dihydro indolyl.

“Heteroarylalkyl” refers to a heteroaryl as described above joined by way of an alkyl as described above (i.e., -alkyl-heteroaryl). Examples of heteroarylalkyl include, but are not limited to,

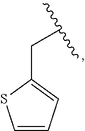 [SMILES: *Cc1cccs1]

and the like.

“Alkylheteroaryl” refers to an alkyl as described above joined by way of a heteroaryl as described above (i.e., -heteroaryl-alkyl). Examples of heteroarylalkyl include, but are not limited to,

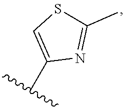 [SMILES: *c1csc(C)n1]

and the like.

The phrase “such that all valencies are satisfied” is readily understood by a person of skill in the art. For example in structure (I) “such that all valencies are satisfied” with respect to ring A means that ring A is connected to ring D and ring A is optionally connected to one or more R₁ substituents such that all valencies are satisfied.

“Isomer” is used herein to encompass all chiral, diastereomeric or racemic forms of a structure (also referred to as a stereoisomer, as opposed to a structural or positional isomer), unless a particular stereochemistry or isomeric form is specifically indicated. Such compounds can be enriched or resolved optical isomers at any or all asymmetric atoms as are apparent from the depictions, at any degree of enrichment. Both racemic and diastereomeric mixtures, as well as the individual optical isomers can be synthesized so as to be substantially free of their enantiomeric or diastereomeric partners, and these are all within the scope of certain embodiments of the invention. The isomers resulting from the presence of a chiral center comprise a pair of nonsuperimposable-isomers that are called “enantiomers.” Single enantiomers of a pure compound are optically active (i.e., they are capable of rotating the plane of plane polarized light and are designated R or S).

“Isolated optical isomer” means a compound which has been substantially purified from the corresponding optical isomer(s) of the same formula. For example, the isolated isomer may be at least about 80%, at least 80% or at least 85% pure by weight. In other embodiments, the isolated isomer is at least 90% pure or at least 98% pure, or at least 99% pure by weight.

“Substantially enantiomerically or diastereomerically” pure means a level of enantiomeric or diastereomeric enrichment of one enantiomer with respect to the other enantiomer or diastereomer of at least about 80%, and more specifically in excess of 80%, 85%, 90%, 95%, 98%, 99%, 99.5% or 99.9%.

The terms “racemate” and “racemic mixture” refer to an equal mixture of two enantiomers. A racemate is labeled “(±)” because it is not optically active (i.e., will not rotate plane-polarized light in either direction since its constituent enantiomers cancel each other out). All compounds with an asterisk (*) adjacent to a tertiary or quaternary carbon are optically active isomers, which may be purified from the respective racemate and/or synthesized by appropriate chiral synthesis.

A “hydrate” is a compound that exists in combination with water molecules. The combination can include water in stoichiometric quantities, such as a monohydrate or a dihydrate, or can include water in random amounts. As the term is used herein a “hydrate” refers to a solid form; that is, a compound in a water solution, while it may be hydrated, is not a hydrate as the term is used herein.

A “solvate” is similar to a hydrate except that a solvent other than water is present. For example, methanol or ethanol can form an “alcoholate”, which can again be stoichiometric or non-stoichiometric. As the term is used herein a “solvate” refers to a solid form; that is, a compound in a solvent solution, while it may be solvated, is not a solvate as the term is used herein.

“Isotope” refers to atoms with the same number of protons but a different number of neutrons, and an isotope of a compound of structure (I) includes any such compound wherein one or more atoms are replaced by an isotope of that atom. For example, carbon 12, the most common form of carbon, has six protons and six neutrons, whereas carbon 13 has six protons and seven neutrons, and carbon 14 has six protons and eight neutrons. Hydrogen has two stable isotopes, deuterium (one proton and one neutron) and tritium (one proton and two neutrons). While fluorine has a number of isotopes, fluorine-19 is longest-lived. Thus, an isotope of a compound having the structure of structure (I) includes, but not limited to, compounds of structure (I) wherein one or more carbon 12 atoms are replaced by carbon-13 and/or carbon-14 atoms, wherein one or more hydrogen atoms are replaced with deuterium and/or tritium, and/or wherein one or more fluorine atoms are replaced by fluorine-19.

“Salt” generally refers to an organic compound, such as a carboxylic acid or an amine, in ionic form, in combination with a counter ion. For example, salts formed between acids in their anionic form and cations are referred to as “acid addition salts”. Conversely, salts formed between bases in the cationic form and anions are referred to as “base addition salts.”

The term “pharmaceutically acceptable” refers to an agent that has been approved for human consumption and is generally non-toxic. For example, the term “pharmaceutically acceptable salt” refers to nontoxic inorganic or organic acid and/or base addition salts (see, e.g., Lit et al., Salt Selection for Basic Drugs, Int. J. Pharm., 33, 201-217, 1986) (incorporated by reference herein).

Pharmaceutically acceptable base addition salts of compounds of the invention include, for example, metallic salts including alkali metal, alkaline earth metal, and transition metal salts such as, for example, calcium, magnesium, potassium, sodium, and zinc salts. Pharmaceutically acceptable base addition salts also include organic salts made from basic amines such as, for example, N,N′dibenzylethylenediamine, chloroprocaine, choline, diethanolamine, ethylenediamine, meglumine (N-methylglucamine), and procaine.

Pharmaceutically acceptable acid addition salts may be prepared from an inorganic acid or from an organic acid. Examples of inorganic acids include hydrochloric, hydrobromic, hydroiodic, nitric, carbonic, sulfuric, and phosphoric acids. Appropriate organic acids may be selected from aliphatic, cycloaliphatic, aromatic, aromatic aliphatic, heterocyclic, carboxylic, and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, hippuric, malonic, oxalic, embonic (pamoic), methanesulfonic, ethanesulfonic, benzenesulfonic, panthothenic, trifluoromethanesulfonic, 2-hydroxyethanesulfonic, p-toluenesulfonic, sulfanilic, cyclohexylaminosulfonic, stearic, alginic, βhydroxybutyric, salicylic, -galactaric, and galacturonic acid.

The compounds of the disclosure (i.e., compounds of structure (I) and embodiments thereof), or their pharmaceutically acceptable salts may contain one or more centers of geometric asymmetry and may thus give rise to enantiomers, diastereomers, and other stereoisomeric forms that are defined, in terms of absolute stereochemistry, as (R)- or (S)- or, as (D)- or (L)- for amino acids. Embodiments thus include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also included.

Although pharmaceutically unacceptable salts are not generally useful as medicaments, such salts may be useful, for example as intermediates in the synthesis of compounds having the structure (I), for example in their purification by recrystallization.

As used herein, the phrase “MRGPRD or MRGPRD ortholog dependent condition” means a condition where the activation, over sensitization, or desensitization of MRGPRD or its ortholog by a natural or synthetic ligand initiates, mediates, sustains, or augments a pathological condition.

In some embodiments, the MRGPRD dependent condition is a pain associated condition, an itch associated condition, an inflammatory condition, an ocular associated condition, a cardiovascular and renal disease associated condition, an inflammatory or autoimmune disorder, or a cognitive impairment associate condition.

As used herein, the phrase “pain associated condition” means any pain due to a medical condition. Thus, in one embodiment, the method of present invention is provided to treat a pain associated condition, such as Acute Pain, Advanced Prostate Cancer, AIDS-Related Pain, Ankylosing Spondylitis, Arachnoiditis, Arthritis, Arthrofibrosis, Ataxic Cerebral Palsy, Autoimmune Atrophic Gastritis, Avascular Necrosis, Back Pain, Behcet's Disease (Syndrome), Burning Mouth Syndrome, Bursitis, Cancer Pain, Carpal Tunnel, Cauda Equina Syndrome, Central Pain Syndrome, Cerebral Palsy, Cervical Stenosis, Charcot-Marie-Tooth (CMT) Disease, Chronic Fatigue Syndrome (CFS), Chronic Functional Abdominal Pain (CFAP), Chronic Pain, Chronic Pancreatitis, Chronic Pelvic Pain Syndrome, Collapsed Lung (Pneumothorax), Complex Regional Pain Syndrome (RSD), Constipation, Corneal Neuropathic Pain, Crohn's Disease, Degenerative Disc Disease, Dental Pain, Dercum's Disease, Dermatomyositis, Diabetic Peripheral Neuropathy (DPN), Dry Eye Syndrome, Dystonia, Ehlers-Danlos Syndrome (EDS), Endometriosis, Eosinophilia-Myalgia Syndrome (EMS), Erythromelalgia, Fibromyalgia, Gout, Headaches, Herniated disc, Hydrocephalus, Inflammatory bowel disease (IBD), Intercostal Neuraligia, Interstitial Cystitis, Irritable Bowel syndrome (IBS), Juvenile Dermatositis (Dermatomyositis), Knee Injury, Leg Pain, Loin Pain-Haematuria Syndrome, Lupus, Lyme Disease, Medullary Sponge Kidney (MSK), Meralgia Paresthetica, Mesothelioma, Migraine, Musculoskeletal pain, Myofascial Pain, Myositis, Neck Pain, Neuropathic Pain, Occipital Neuralgia, Occular Itch, Osteoarthritis, Paget's Disease, Parsonage Turner Syndrome, Pelvic Pain, Periodontitis Pain, Peripheral Neuropathy, Phantom Limb Pain, Pinched Nerve, Polycystic Kidney Disease, Polymyalgia Rhuematica, Polymyositis, Porphyria, Post Herniorraphy Pain Syndrome, Post Mastectomy, Postoperative Pain, Pain Syndrome, Post Stroke Pain, Post Thorocotomy Pain Syndrome, Postherpetic Neuralgia (Shingles), Post-Polio Syndrome, Primary Lateral Sclerosis, Psoriatic Arthritis, Pudendal Neuralgia, Radiculopathy, Raynaud's Disease, Rheumatoid Arthritis (RA), Sacroiliac Joint Dysfunction, Sarcoidosi, Scheuemann's Kyphosis Disease, Sciatica, Scoliosis, Shingles (Herpes Zoster), Sjogren's Syndrome, Spasmodic Torticollis, Sphincter of Oddi Dysfunction, Spinal Cerebellum Ataxia (SCA Ataxia), Spinal Cord Injury, Spinal Stenosis, Syringomyelia, Tarlov Cysts, Transverse Myelitis, Trigeminal Neuralgia, Neuropathic Pain, Ulcerative Colitis, Vascular Pain and Vulvodynia.

As used herein, the phrase “itch associated condition” means pruritus (including acute and chronic pruritus) associated with any condition. The itch sensation can originate, e.g., from the peripheral nervous system (e.g., dermal or neuropathic itch) or from the central nervous system (e.g., neuropathic, neurogenic or psychogenic itch). Thus, in one embodiment, the method of present invention is provided to treat an itch associated condition, such as chronic itch; contact dermatitis; Allergic blepharitis; Anaphylaxis; Anaphylactoid drug reactions; Anaphylactic shock; Anemia; Atopic dermatitis; Bullous pemphigoid; Candidiasis; Chicken pox; end-stage renal failure; hemodialysis; Cholestatic pruritis; Chronic urticaria; Contact dermatitis, Atopic Dermatitis; Dermatitis herpetiformis; Diabetes; Drug allergy, Dry skin; Dyshidrotic dermatitis; Ectopic eczema; Eosinophilic fasciitis; Epidermolysis bullosa; Erythrasma; Food allergy; Folliculitis; Fungal skin infection; Hemorrhoids; Herpes; HIV infection; Hodgkin's disease; Hyperthyroidism; Iodinated contrast dye allergy; Iron deficiency anemia; Kidney disease; Leukemia, Porphyrias; Lymphoma; Mast cell activation syndrome; Malignancy; Mastocystosis; Multiple myeloma; Neurodermatitis; Onchocerciasis; Paget's disease; Pediculosis; Polycythemia rubra vera; Prurigo nodularis; Lichen Planus; Lichen Sclerosis; Pruritus ani; Pseudo-allergic reactions; Pseudorabies; Psoriasis; Rectal prolapse; Sarcoidosis granulomas; Scabies; Schistosomiasis; Scleroderma, Severe stress, Stasia dermatitis; Swimmer's itch; Thyroid disease; Tinea cruris; Uremic Pruritus; Rosacea; Cutaneous amyloidosis; Scleroderma; Acne; wound healing; burn healing; ocular itch; and Urticaria.

As used herein, the term “administration” refers to providing a compound, or a pharmaceutical composition comprising the compound as described herein. The compound or composition can be administered by another person to the subject or it can be self-administered by the subject. Non-limiting examples of routes of administration are oral, parenteral (e.g., intravenous), or topical.

As used herein, the term “treatment” refers to an intervention that ameliorates a sign or symptom of a disease or pathological condition. As used herein, the terms “treatment”, “treat” and “treating,” with reference to a disease, pathological condition or symptom, also refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A prophylactic treatment is a treatment administered to a subject who does not exhibit signs of a disease or exhibits only early signs, for the purpose of decreasing the risk of developing pathology. A therapeutic treatment is a treatment administered to a subject after signs and symptoms of the disease have developed.

As used herein, the term “subject” refers to an animal (e.g., a mammal, such as a human, dog or horse). A subject to be treated according to the methods described herein may be one who has been diagnosed with a MRGPRD dependent condition or MRGPRD ortholog dependent condition, such as a pain associated condition. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition. The term “patient” may be used interchangeably with the term “subject.” A subject may refer to an adult or pediatric subject.

The Federal Food, Drug, and Cosmetic Act defines “pediatric” as a subject aged 21 or younger at the time of their diagnosis or treatment. Pediatric subpopulations are further characterized as: (i) neonates—from birth through the first 28 days of life; (ii) infants—from 29 days to less than 2 years; (iii) children—2 years to less than 12 years; and (iv) adolescents—aged 12 through 21. Despite the definition, depending on the susceptible patient population and clinical trial evaluation, an approved regulatory label may include phrasing that specifically modifies the range of a pediatric population, such as, for example, pediatric patients up to 22 years of age.

In another embodiment, the method of treating a subject having a MRGPRD dependent condition (e.g., a pain associated conditions) described herein further comprises administering to the subject a pharmaceutically effective amount of a second therapeutic agent. In another embodiment, a method of treating a subject having a pain associated condition is provided, the method comprising administering to the subject a pharmaceutically effective amount of a compound having structure (I) or a pharmaceutically acceptable salt, isomer, hydrate, solvate or isotope thereof, or a pharmaceutical composition thereof.

### Methods

### MRGPRD Modulators

In some embodiments, compounds having activity as modulators of the Mas-related G-protein coupled receptor D are provided, the compounds having the following structure (I):

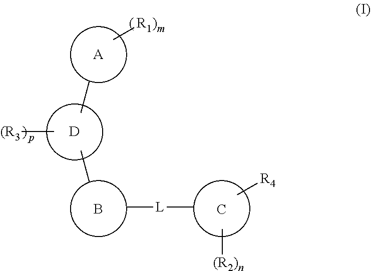

or a pharmaceutically acceptable salt, isomer, hydrate, solvate, or isotope thereof, wherein:
A and B are each individually carbocyclyl or heterocyclyl;
C is phenyl or C is optionally absent when R₄ is aminylalkyl;
D is

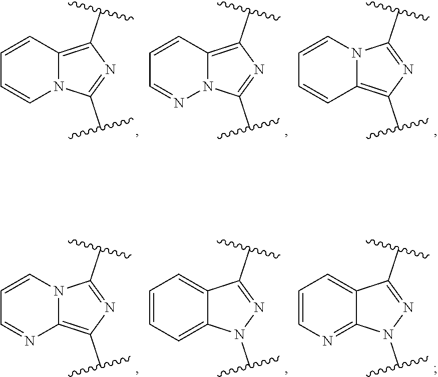

L is a linker having the structure —C(O)—, —C(O)-alkyl-, —C(O)—NRa-, —C(O)—NRa-alkyl-, —S(O)₂—, or —S(O)₂-alkyl-;
R₁ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, alkoxycarbonyl, aminylcarbonyl, alkylsulfonyl, cycloalkylalkyl, aryl, aralkyl, or arylsulfonyl;
R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
R₄ is aminyl, aminylalkyl, or aminylcarbonyl;
Ra is H or alkyl;
m is 0, 1, or 2;
n is 0, 1, 2, or 3; and
p is 0, 1, 2, or 3.

In some embodiments, compounds of structure (I) are provided wherein A is carbocyclyl. In other embodiments, compounds of structure (I) are provided wherein A is heterocyclyl. In certain embodiments, compounds of structure (I) are provided wherein A is selected from:

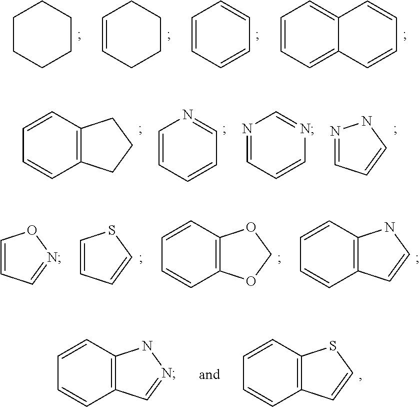

such that all valencies are satisfied. In specific embodiments, compounds of structure (I) are provided wherein A is selected from:

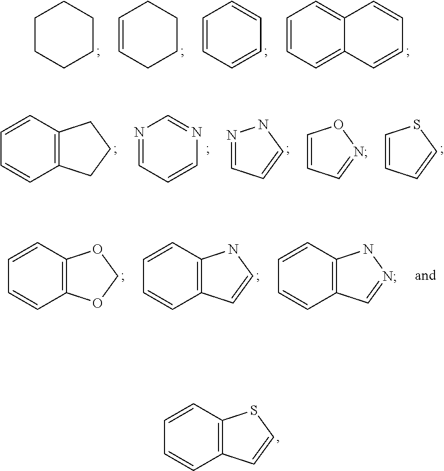

such that all valencies are satisfied.

In some embodiments, compounds of structure (I) are provided wherein B is carbocyclyl. In certain embodiments, compounds of structure (I) are provided wherein B is aryl. In specific embodiments, compounds of structure (I) are provided wherein B is phenyl. In other embodiments, compounds of structure (I) are provided wherein B is heterocyclyl. In other embodiments, compounds of structure (I) are provided wherein B is heteroaryl. In certain embodiments, compounds of structure (I) are provided wherein B is thiophenyl. In additional embodiments, compounds of structure (I) are provided wherein B is piperidinyl. In some embodiments, compounds of structure (I) are provided wherein B is pyrrolidinyl. In other embodiments, compounds of structure (I) are provided wherein B is azetidinyl.

In some embodiments, compounds of structure (I) are provided wherein A is carbocyclyl and B is heterocyclyl. In certain embodiments, compounds of structure (I) are provided wherein A is phenyl and B is piperidinyl, pyrrolidinyl, or azetidinyl. In other embodiments, compounds of structure (I) are provided wherein A is carbocyclyl and B is carbocyclyl. In certain embodiments, compounds of structure (I) are provided wherein A is aryl and B is aryl. In specific embodiments, compounds of structure (I) are provided wherein A is phenyl and B is phenyl. In additional embodiments, compounds of structure (I) are provided wherein A is heterocyclyl and B is heterocyclyl. In other embodiments, compounds of structure (I) are provided wherein A is heteroaryl and B is heterocyclyl. In certain embodiments, compounds of structure (I) are provided wherein A is heteroaryl and B is piperidinyl, pyrrolidinyl, or azetidinyl.

In some embodiments, compounds of structure (I) are provided wherein C is aryl. In some embodiments, compounds of structure (I) are provided wherein C is phenyl. In other embodiments, compounds of structure (I) are provided wherein C is absent and R₄ is aminylalkyl.

In some embodiments, compounds of structure (I) are provided wherein D is

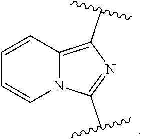 [SMILES: *c1nc(*)n2ccccc12]

In other embodiments, compounds of structure (I) are provided wherein D is

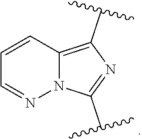 [SMILES: *c1nc(*)n2ncccc12]

In additional embodiments, compounds of structure (I) are provided wherein D is

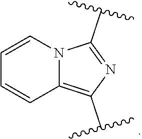 [SMILES: *c1nc(*)n2ccccc12]

In further embodiments, compounds of structure (I) are provided wherein D is

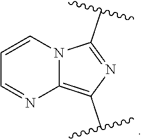 [SMILES: *c1nc(*)n2cccnc12]

In yet other embodiments, compounds of structure (I) are provided wherein D is

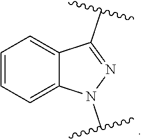 [SMILES: *c1nn(*)c2ccccc12]

In some embodiments, compounds of structure (I) are provided wherein D is

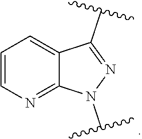 [SMILES: *c1nn(*)c2ncccc12]

In some embodiments, compounds of structure (I) are provided wherein L is a linker having the structure —C(O)—. In other embodiments, compounds of structure (I) are provided wherein L is a linker having the structure —C(O)-alkyl-. In additional embodiments, compounds of structure (I) are provided wherein L is a linker having the structure —C(O)—NRa-. In further embodiments, compounds of structure (I) are provided wherein L is a linker having the structure —C(O)—NRa-alkyl-. In yet other embodiments, compounds of structure (I) are provided wherein L is a linker having the structure —S(O)₂—. In some embodiments, compounds of structure (I) are provided wherein L is a linker having the structure —S(O)₂-alkyl-.

In some embodiments, compounds of structure (I) are provided wherein R₁ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, alkoxycarbonyl, aminylcarbonyl, or alkylsulfonyl. In other embodiments, compounds of structure (I) are provided wherein R₁ is cycloalkylalkyl, aryl, aralkyl, or arylsulfonyl. In certain embodiments, compounds of structure (I) are provided wherein R₁ is selected from:

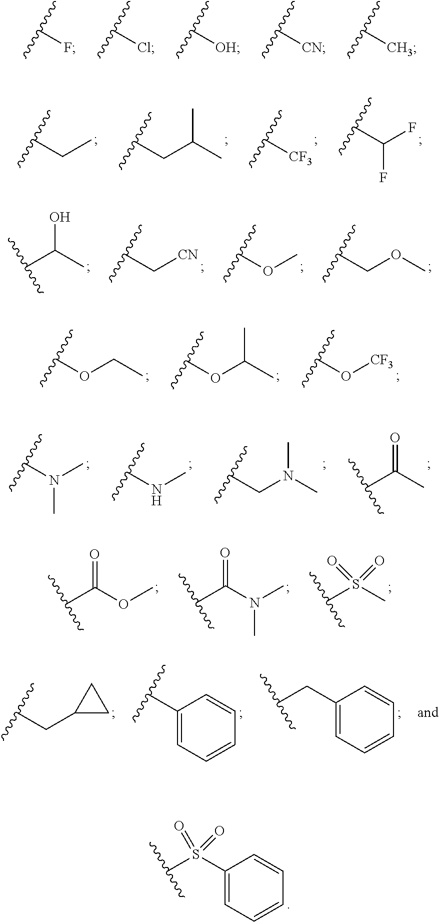

In some embodiments, compounds of structure (I) are provided wherein R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, or alkylsufonylalkyl. In some embodiments, compounds of structure (I) are provided wherein R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, or alkylsulfonyl. In other embodiments, compounds of structure (I) are provided wherein R₂ is aryl, heterocyclyl, or alkylheteroaryl. In certain embodiments, compounds of structure (I) are provided wherein R₂ is selected from:

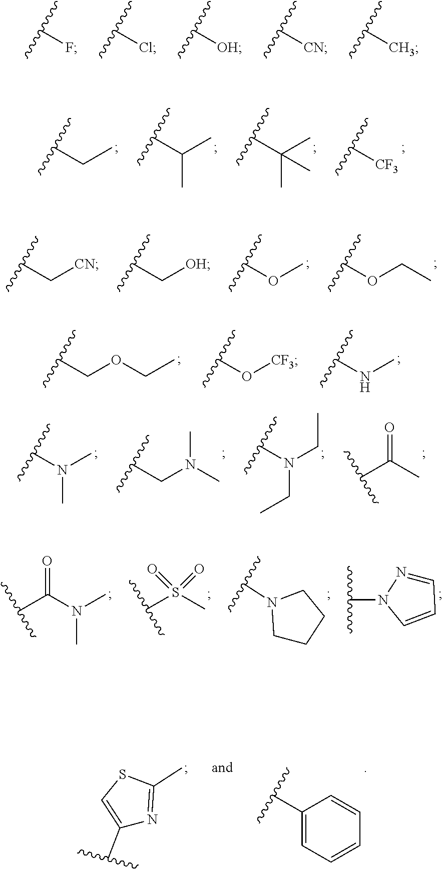

In some embodiments, compounds of structure (I) are provided wherein R₂ is selected from halo and alkyl. In specific embodiments, compounds of structure (I) are provided wherein R₂ is selected from:

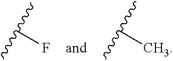

In certain embodiments, compounds of structure (I) are provided wherein R₃ is alkyl. In other embodiments, compounds of structure (I) are provided wherein R₃ is alkoxy.

In some embodiments, compounds of structure (I) are provided wherein R₄ is aminyl. In other embodiments, compounds of structure (I) are provided wherein R₄ is aminylalkyl. In additional embodiments, compounds of structure (I) are provided wherein R₄ is aminylcarbonyl.

In certain embodiments, compounds of structure (I) are provided wherein R₄ is

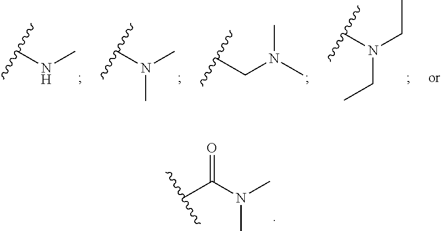

In specific embodiments, compounds of structure (I) are provided wherein R₄ is

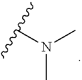 [SMILES: *N(C)C]

In some embodiments, compounds of structure (I) are provided wherein Ra is H. In other embodiments, compounds of structure (I) are provided wherein Ra is alkyl.

In some embodiments, compounds of structure (I) are provided wherein m is 0. In other embodiments, compounds of structure (I) are provided wherein m is 1. In additional embodiments, compounds of structure (I) are provided wherein m is 2.

In some embodiments, compounds of structure (I) are provided wherein n is 0. In other embodiments, compounds of structure (I) are provided wherein n is 1. In additional embodiments, compounds of structure (I) are provided wherein n is 2. In further embodiments, compounds of structure (I) are provided wherein n is 3.

In some embodiments, compounds of structure (I) are provided wherein p is 0. In other embodiments, compounds of structure (I) are provided wherein p is 1. In additional embodiments, compounds of structure (I) are provided wherein p is 2. In further embodiments, compounds of structure (I) are provided wherein p is 3.

Representative compounds of structure (I) include any one of the compounds listed in Table I below, as well as a pharmaceutically acceptable isomer, racemate, hydrate, solvate, isotope, or salt thereof. To this end, representative compounds are identified herein by their respective “Compound Number”, which is sometimes abbreviated as “Compound No.” or “Cpd. No.”

TABLE I
Compounds of Structure I

| Cpd. No. | Structure |
|---|---|
| 1-1 | 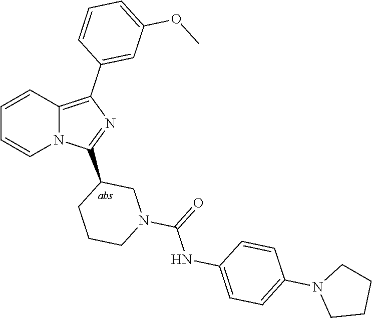 [SMILES: COc1cccc(-c2nc([C@@H]3CCCN(C(=O)Nc4ccc(N5CCCC5)cc4)C3)n3ccccc23)c1] |
| 1-2 | 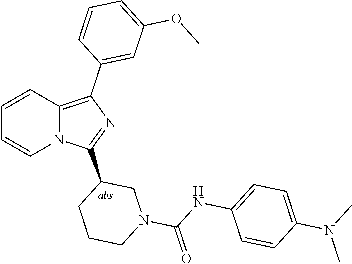 [SMILES: COc1cccc(-c2nc([C@@H]3CCCN(C(=O)Nc4ccc(N(C)C)cc4)C3)n3ccccc23)c1] |
| 1-5 | 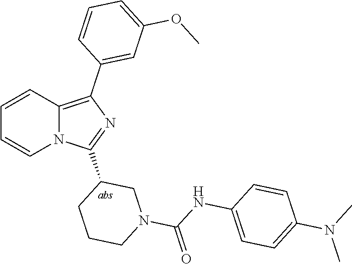 [SMILES: COc1cccc(-c2nc([C@H]3CCCN(C(=O)Nc4ccc(N(C)C)cc4)C3)n3ccccc23)c1] |
| 1-6 | 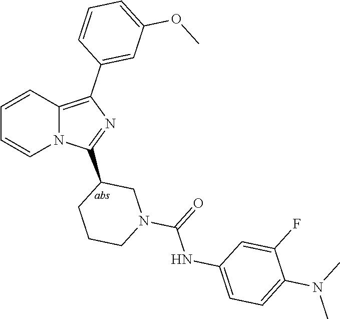 [SMILES: COc1cccc(-c2nc([C@@H]3CCCN(C(=O)Nc4ccc(N(C)C)c(F)c4)C3)n3ccccc23)c1] |
| 1-7 | 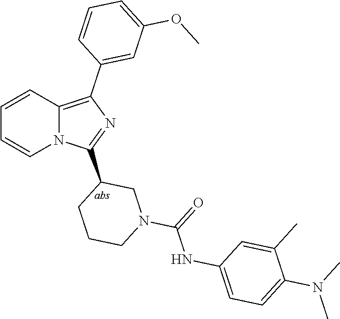 [SMILES: COc1cccc(-c2nc([C@@H]3CCCN(C(=O)Nc4ccc(N(C)C)c(C)c4)C3)n3ccccc23)c1] |
| 1-8 | 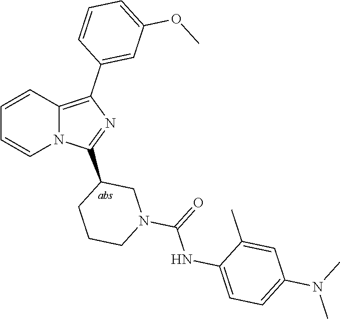 [SMILES: COc1cccc(-c2nc([C@@H]3CCCN(C(=O)Nc4ccc(N(C)C)cc4C)C3)n3ccccc23)c1] |
| 1-19 | 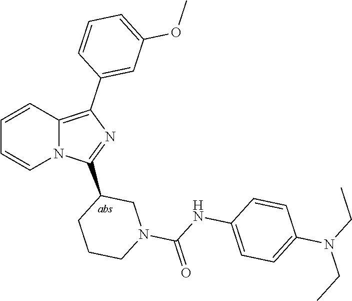 [SMILES: CCN(CC)c1ccc(NC(=O)N2CCC[C@@H](c3nc(-c4cccc(OC)c4)c4ccccn34)C2)cc1] |
| 1-20 | 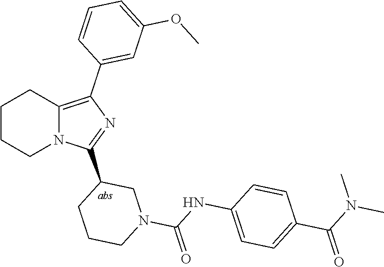 [SMILES: COc1cccc(-c2nc([C@@H]3CCCN(C(=O)Nc4ccc(C(=O)N(C)C)cc4)C3)n3c2CCCC3)c1] |
| 1-21 | 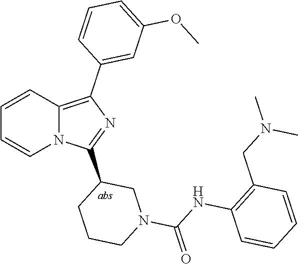 [SMILES: COc1cccc(-c2nc([C@@H]3CCCN(C(=O)Nc4ccccc4CN(C)C)C3)n3ccccc23)c1] |
| 1-26 | 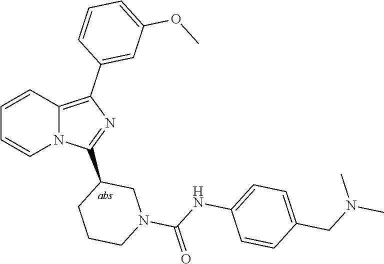 [SMILES: COc1cccc(-c2nc([C@@H]3CCCN(C(=O)Nc4ccc(CN(C)C)cc4)C3)n3ccccc23)c1] |
| 1-27 | 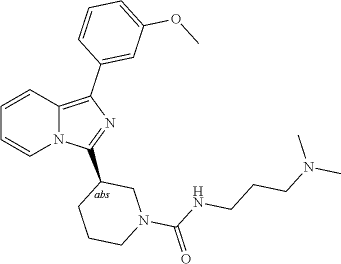 [SMILES: COc1cccc(-c2nc([C@@H]3CCCN(C(=O)NCCCN(C)C)C3)n3ccccc23)c1] |
| 1-29 | 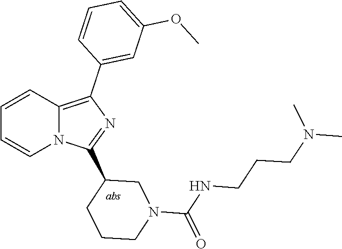 [SMILES: COc1cccc(-c2nc([C@@H]3CCCN(C(=O)NCCCN(C)C)C3)n3ccccc23)c1] |
| 3-24 | 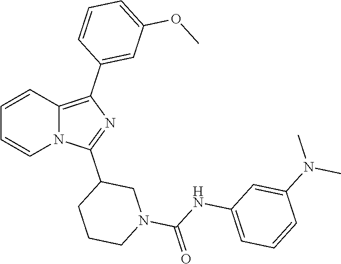 [SMILES: COc1cccc(-c2nc(C3CCCN(C(=O)Nc4cccc(N(C)C)c4)C3)n3ccccc23)c1] |
| 3-25 | 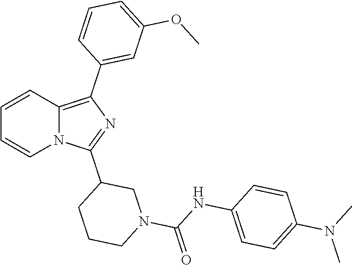 [SMILES: COc1cccc(-c2nc(C3CCCN(C(=O)Nc4ccc(N(C)C)cc4)C3)n3ccccc23)c1] |
| 4-3 | 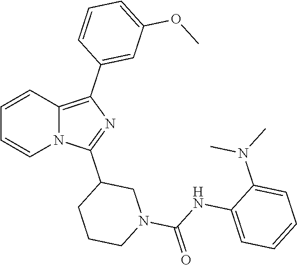 [SMILES: COc1cccc(-c2nc(C3CCCN(C(=O)Nc4ccccc4N(C)C)C3)n3ccccc23)c1] |
| 5-4 | 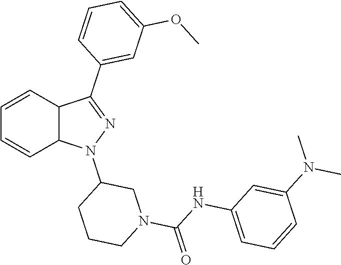 [SMILES: COc1cccc(C2=NN(C3CCCN(C(=O)Nc4cccc(N(C)C)c4)C3)C3C=CC=CC23)c1] |
| 5-6 | 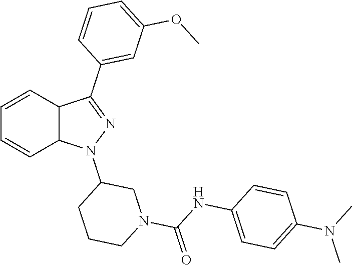 [SMILES: COc1cccc(C2=NN(C3CCCN(C(=O)Nc4ccc(N(C)C)cc4)C3)C3C=CC=CC23)c1] |
| 5-8 | 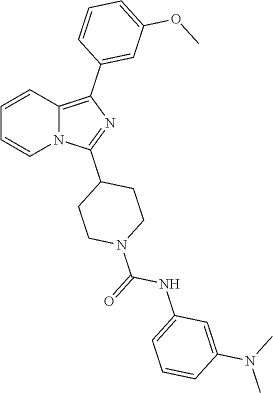 [SMILES: COc1cccc(-c2nc(C3CCN(C(=O)Nc4cccc(N(C)C)c4)CC3)n3ccccc23)c1] |
| 5-9 | 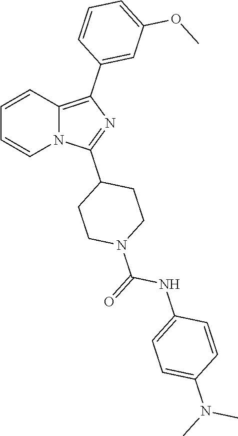 [SMILES: COc1cccc(-c2nc(C3CCN(C(=O)Nc4ccc(N(C)C)cc4)CC3)n3ccccc23)c1] |
| 5-17 | 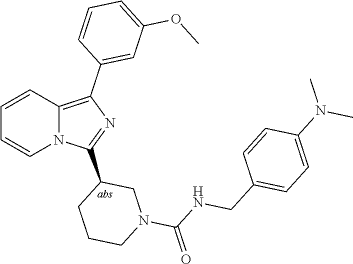 [SMILES: COc1cccc(-c2nc([C@@H]3CCCN(C(=O)NCc4ccc(N(C)C)cc4)C3)n3ccccc23)c1] |
| 5-18 | 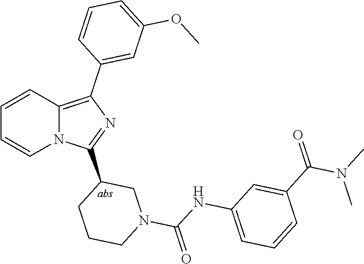 [SMILES: COc1cccc(-c2nc([C@@H]3CCCN(C(=O)Nc4cccc(C(=O)N(C)C)c4)C3)n3ccccc23)c1] |
| 5-19 | 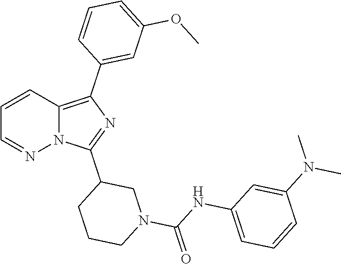 [SMILES: COc1cccc(-c2nc(C3CCCN(C(=O)Nc4cccc(N(C)C)c4)C3)n3ncccc23)c1] |
| 5-20 | 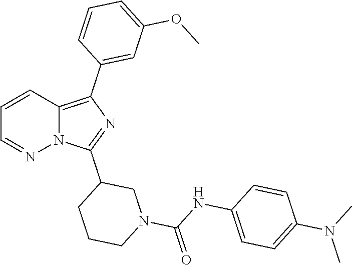 [SMILES: COc1cccc(-c2nc(C3CCCN(C(=O)Nc4ccc(N(C)C)cc4)C3)n3ncccc23)c1] |
| 5-31 | 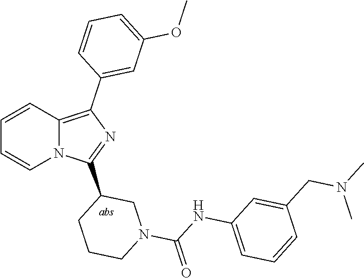 [SMILES: COc1cccc(-c2nc([C@@H]3CCCN(C(=O)Nc4cccc(CN(C)C)c4)C3)n3ccccc23)c1] |
| 5-33 | 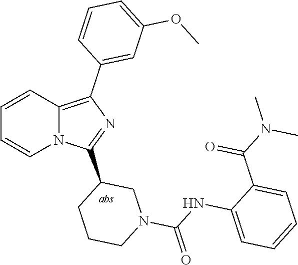 [SMILES: COc1cccc(-c2nc([C@@H]3CCCN(C(=O)Nc4ccccc4C(=O)N(C)C)C3)n3ccccc23)c1] |
| 5-36 | 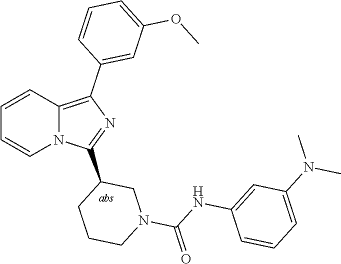 [SMILES: COc1cccc(-c2nc([C@@H]3CCCN(C(=O)Nc4cccc(N(C)C)c4)C3)n3ccccc23)c1] |
| 5-37 | 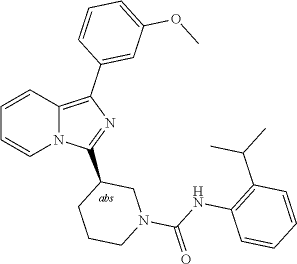 [SMILES: COc1cccc(-c2nc([C@@H]3CCCN(C(=O)Nc4ccccc4C(C)C)C3)n3ccccc23)c1] |
| 7-1 | 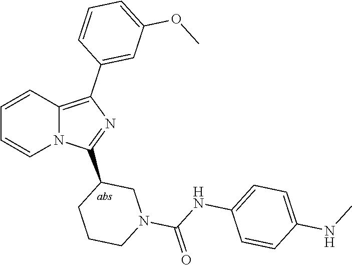 [SMILES: CNc1ccc(NC(=O)N2CCC[C@@H](c3nc(-c4cccc(OC)c4)c4ccccn34)C2)cc1] |
| 8-1 | 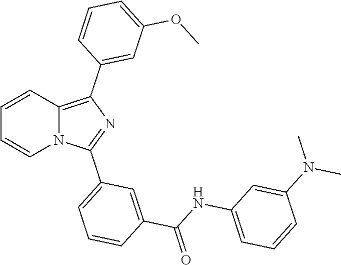 [SMILES: COc1cccc(-c2nc(-c3cccc(C(=O)Nc4cccc(N(C)C)c4)c3)n3ccccc23)c1] |
| 8-2 | 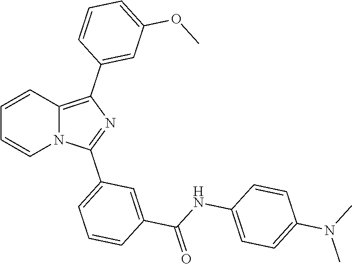 [SMILES: COc1cccc(-c2nc(-c3cccc(C(=O)Nc4ccc(N(C)C)cc4)c3)n3ccccc23)c1] |
| 9-1 | 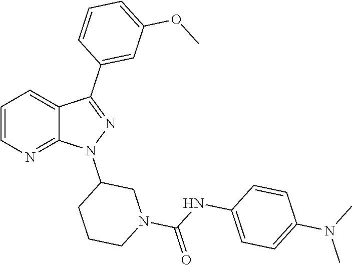 [SMILES: COc1cccc(-c2nn(C3CCCN(C(=O)Nc4ccc(N(C)C)cc4)C3)c3ncccc23)c1] |
| 9-2 | 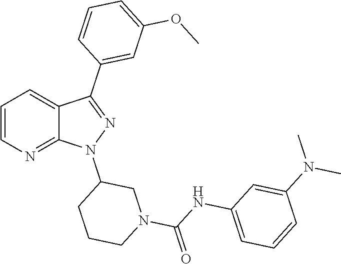 [SMILES: COc1cccc(-c2nn(C3CCCN(C(=O)Nc4cccc(N(C)C)c4)C3)c3ncccc23)c1] |
| 10-1 | 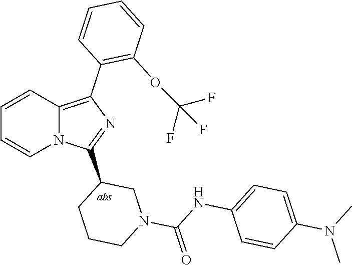 [SMILES: CN(C)c1ccc(NC(=O)N2CCC[C@@H](c3nc(-c4ccccc4OC(F)(F)F)c4ccccn34)C2)cc1] |
| 10-2 | 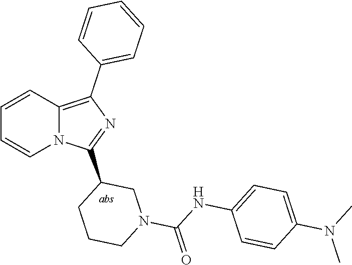 [SMILES: CN(C)c1ccc(NC(=O)N2CCC[C@@H](c3nc(-c4ccccc4)c4ccccn34)C2)cc1] |
| 10-3 | 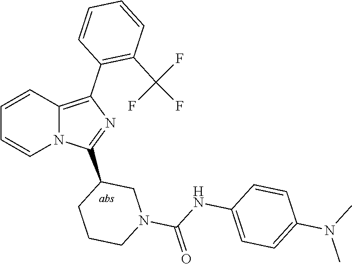 [SMILES: CN(C)c1ccc(NC(=O)N2CCC[C@@H](c3nc(-c4ccccc4C(F)(F)F)c4ccccn34)C2)cc1] |
| 10-4 | 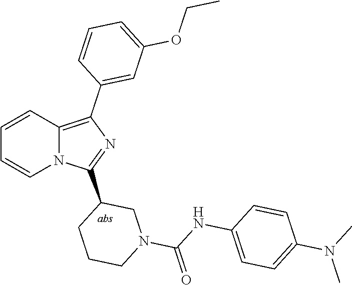 [SMILES: CCOc1cccc(-c2nc([C@@H]3CCCN(C(=O)Nc4ccc(N(C)C)cc4)C3)n3ccccc23)c1] |
| 10-5 | 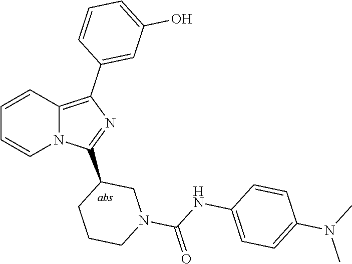 [SMILES: CN(C)c1ccc(NC(=O)N2CCC[C@@H](c3nc(-c4cccc(O)c4)c4ccccn34)C2)cc1] |
| 10-6 | 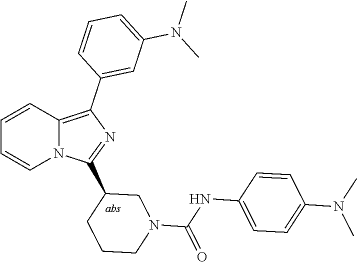 [SMILES: CN(C)c1ccc(NC(=O)N2CCC[C@@H](c3nc(-c4cccc(N(C)C)c4)c4ccccn34)C2)cc1] |
| 10-7 | 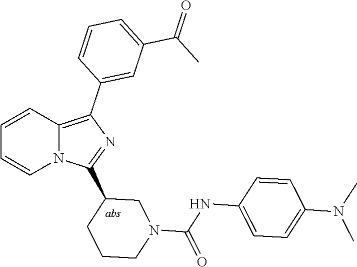 [SMILES: CC(=O)c1cccc(-c2nc([C@@H]3CCCN(C(=O)Nc4ccc(N(C)C)cc4)C3)n3ccccc23)c1] |
| 10-8 | 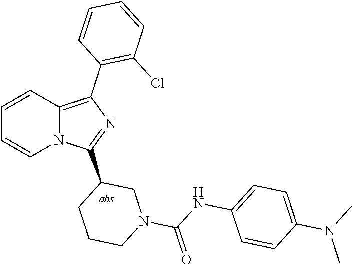 [SMILES: CN(C)c1ccc(NC(=O)N2CCC[C@@H](c3nc(-c4ccccc4Cl)c4ccccn34)C2)cc1] |
| 10-9 | 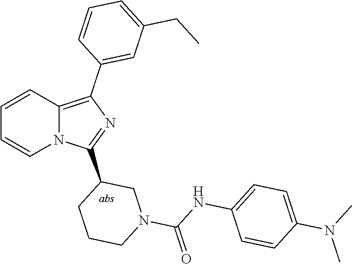 [SMILES: CCc1cccc(-c2nc([C@@H]3CCCN(C(=O)Nc4ccc(N(C)C)cc4)C3)n3ccccc23)c1] |
| 10-10 | 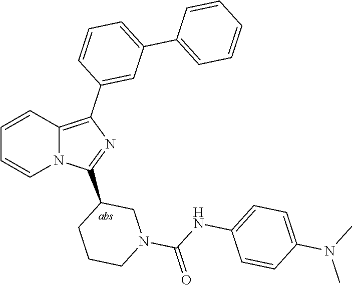 [SMILES: CN(C)c1ccc(NC(=O)N2CCC[C@@H](c3nc(-c4cccc(-c5ccccc5)c4)c4ccccn34)C2)cc1] |
| 10-11 | 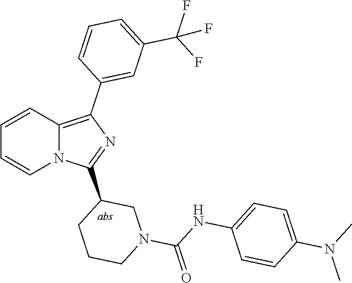 [SMILES: CN(C)c1ccc(NC(=O)N2CCC[C@@H](c3nc(-c4cccc(C(F)(F)F)c4)c4ccccn34)C2)cc1] |
| 10-12 | 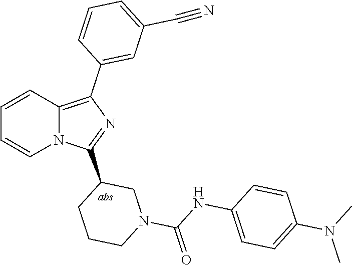 [SMILES: CN(C)c1ccc(NC(=O)N2CCC[C@@H](c3nc(-c4cccc(C#N)c4)c4ccccn34)C2)cc1] |
| 10-13 | 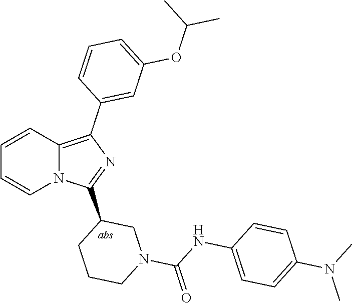 [SMILES: CC(C)Oc1cccc(-c2nc([C@@H]3CCCN(C(=O)Nc4ccc(N(C)C)cc4)C3)n3ccccc23)c1] |
| 10-14 | 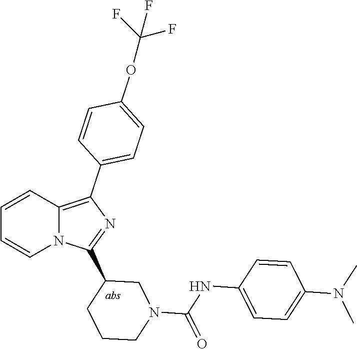 [SMILES: CN(C)c1ccc(NC(=O)N2CCC[C@@H](c3nc(-c4ccc(OC(F)(F)F)cc4)c4ccccn34)C2)cc1] |
| 10-15 | 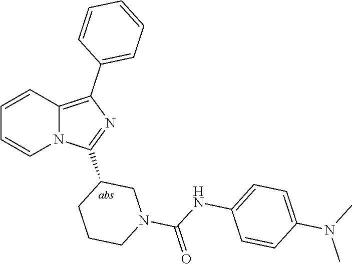 [SMILES: CN(C)c1ccc(NC(=O)N2CCC[C@H](c3nc(-c4ccccc4)c4ccccn34)C2)cc1] |
| 10-16 | 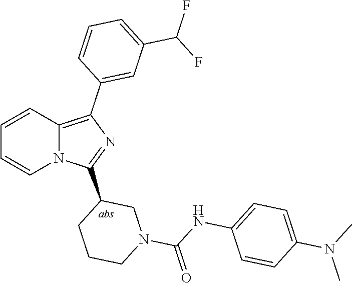 [SMILES: CN(C)c1ccc(NC(=O)N2CCC[C@@H](c3nc(-c4cccc(C(F)F)c4)c4ccccn34)C2)cc1] |
| 10-17 | 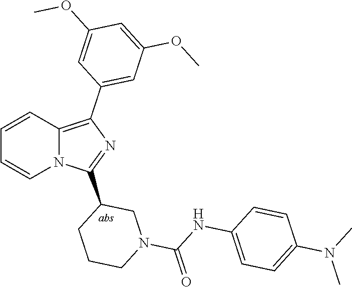 [SMILES: COc1cc(OC)cc(-c2nc([C@@H]3CCCN(C(=O)Nc4ccc(N(C)C)cc4)C3)n3ccccc23)c1] |
| 10-18 | 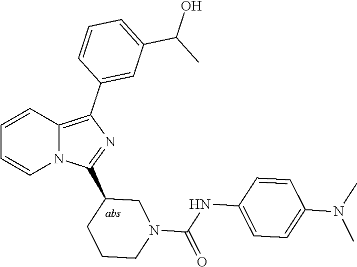 [SMILES: CC(O)c1cccc(-c2nc([C@@H]3CCCN(C(=O)Nc4ccc(N(C)C)cc4)C3)n3ccccc23)c1] |
| 10-19 | 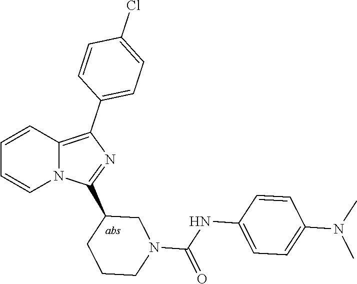 [SMILES: CN(C)c1ccc(NC(=O)N2CCC[C@@H](c3nc(-c4ccc(Cl)cc4)c4ccccn34)C2)cc1] |
| 10-20 | 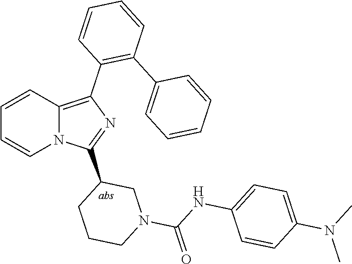 [SMILES: CN(C)c1ccc(NC(=O)N2CCC[C@@H](c3nc(-c4ccccc4-c4ccccc4)c4ccccn34)C2)cc1] |
| 10-21 | 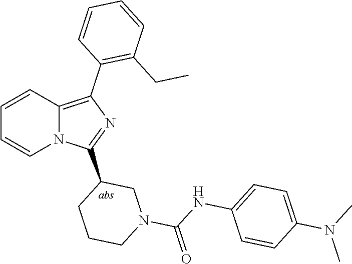 [SMILES: CCc1ccccc1-c1nc([C@@H]2CCCN(C(=O)Nc3ccc(N(C)C)cc3)C2)n2ccccc12] |
| 10-22 | 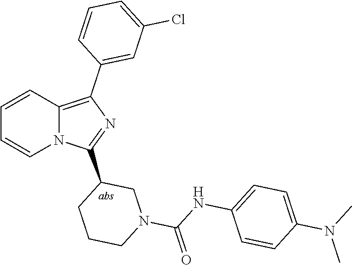 [SMILES: CN(C)c1ccc(NC(=O)N2CCC[C@@H](c3nc(-c4cccc(Cl)c4)c4ccccn34)C2)cc1] |
| 10-23 | 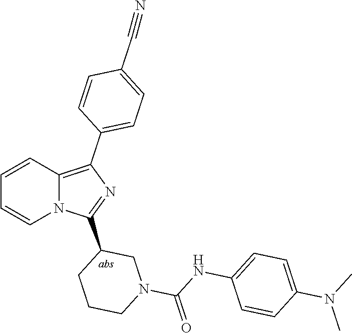 [SMILES: CN(C)c1ccc(NC(=O)N2CCC[C@@H](c3nc(-c4ccc(C#N)cc4)c4ccccn34)C2)cc1] |
| 10-24 | 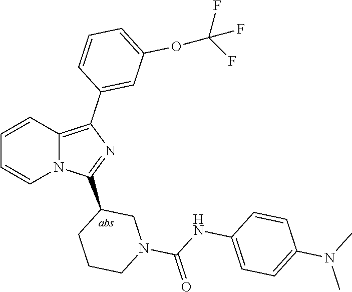 [SMILES: CN(C)c1ccc(NC(=O)N2CCC[C@@H](c3nc(-c4cccc(OC(F)(F)F)c4)c4ccccn34)C2)cc1] |
| 10-26 | 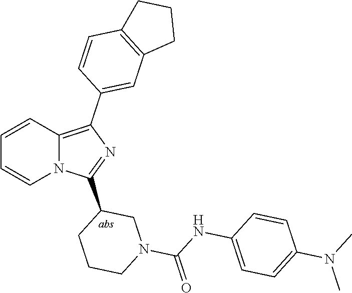 [SMILES: CN(C)c1ccc(NC(=O)N2CCC[C@@H](c3nc(-c4ccc5c(c4)CCC5)c4ccccn34)C2)cc1] |
| 10-27 | 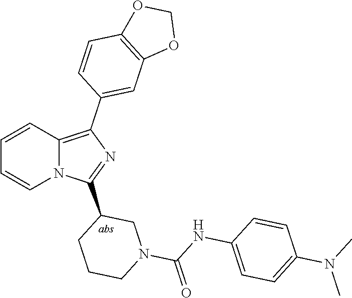 [SMILES: CN(C)c1ccc(NC(=O)N2CCC[C@@H](c3nc(-c4ccc5c(c4)OCO5)c4ccccn34)C2)cc1] |
| 10-28 | 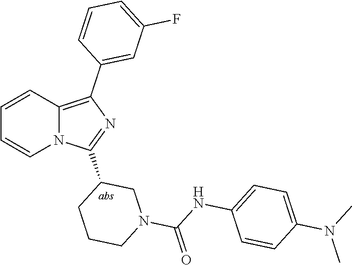 [SMILES: CN(C)c1ccc(NC(=O)N2CCC[C@H](c3nc(-c4cccc(F)c4)c4ccccn34)C2)cc1] |
| 10-29 | 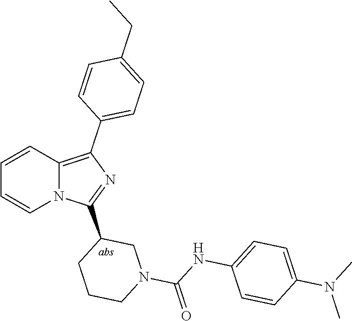 [SMILES: CCc1ccc(-c2nc([C@@H]3CCCN(C(=O)Nc4ccc(N(C)C)cc4)C3)n3ccccc23)cc1] |
| 10-30 | 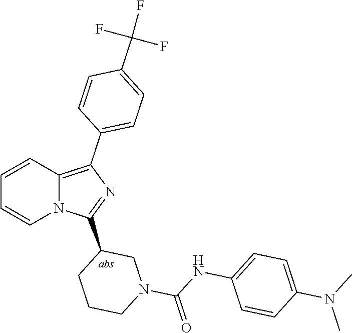 [SMILES: CN(C)c1ccc(NC(=O)N2CCC[C@@H](c3nc(-c4ccc(C(F)(F)F)cc4)c4ccccn34)C2)cc1] |
| 10-31 | 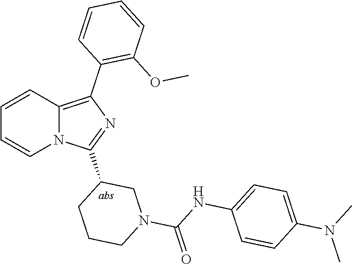 [SMILES: COc1ccccc1-c1nc([C@H]2CCCN(C(=O)Nc3ccc(N(C)C)cc3)C2)n2ccccc12] |
| 10-32 | 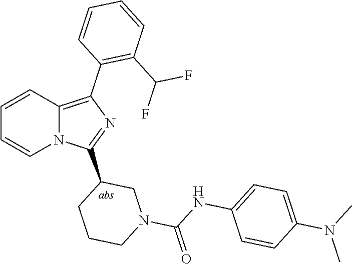 [SMILES: CN(C)c1ccc(NC(=O)N2CCC[C@@H](c3nc(-c4ccccc4C(F)F)c4ccccn34)C2)cc1] |
| 10-33 | 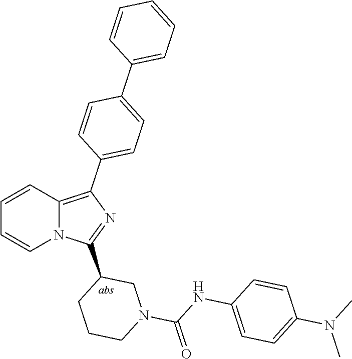 [SMILES: CN(C)c1ccc(NC(=O)N2CCC[C@@H](c3nc(-c4ccc(-c5ccccc5)cc4)c4ccccn34)C2)cc1] |
| 10-34 | 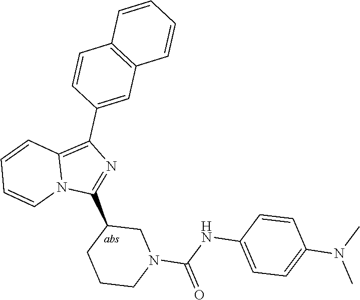 [SMILES: CN(C)c1ccc(NC(=O)N2CCC[C@@H](c3nc(-c4ccc5ccccc5c4)c4ccccn34)C2)cc1] |
| 10-35 | 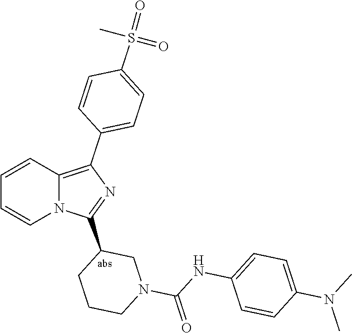 [SMILES: CN(C)c1ccc(NC(=O)N2CCC[C@@H](c3nc(-c4ccc(S(C)(=O)=O)cc4)c4ccccn34)C2)cc1] |
| 10-36 | 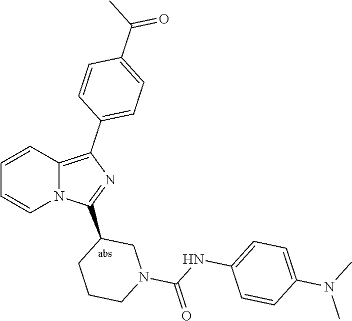 [SMILES: CC(=O)c1ccc(-c2nc([C@@H]3CCCN(C(=O)Nc4ccc(N(C)C)cc4)C3)n3ccccc23)cc1] |
| 10-37 | 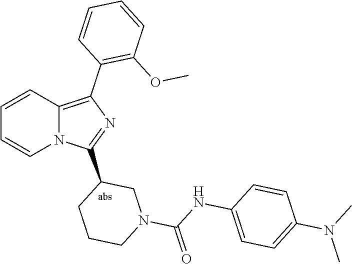 [SMILES: COc1ccccc1-c1nc([C@@H]2CCCN(C(=O)Nc3ccc(N(C)C)cc3)C2)n2ccccc12] |
| 10-40 | 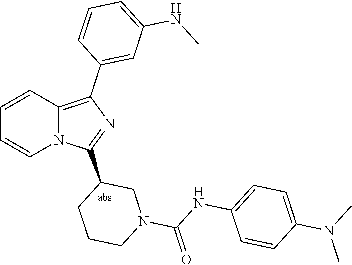 [SMILES: CNc1cccc(-c2nc([C@@H]3CCCN(C(=O)Nc4ccc(N(C)C)cc4)C3)n3ccccc23)c1] |
| 10-42 | 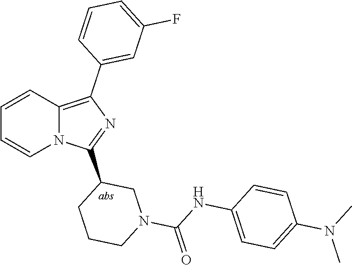 [SMILES: CN(C)c1ccc(NC(=O)N2CCC[C@@H](c3nc(-c4cccc(F)c4)c4ccccn34)C2)cc1] |
| 10-43 | 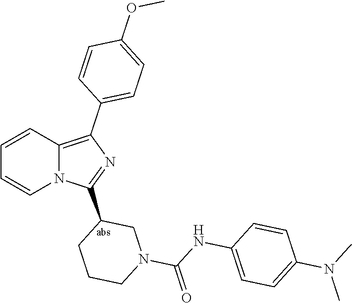 [SMILES: COc1ccc(-c2nc([C@@H]3CCCN(C(=O)Nc4ccc(N(C)C)cc4)C3)n3ccccc23)cc1] |
| 10-44 | 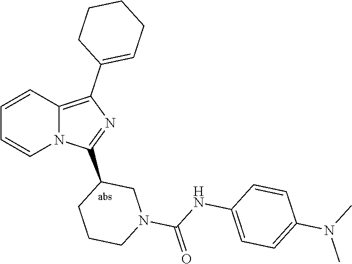 [SMILES: CN(C)c1ccc(NC(=O)N2CCC[C@@H](c3nc(C4=CCCCC4)c4ccccn34)C2)cc1] |
| 10-45 | 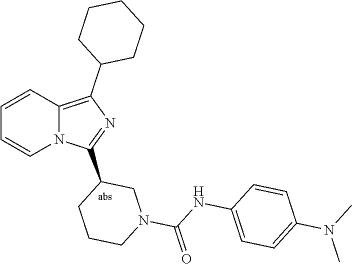 [SMILES: CN(C)c1ccc(NC(=O)N2CCC[C@@H](c3nc(C4CCCCC4)c4ccccn34)C2)cc1] |
| 10-46 | 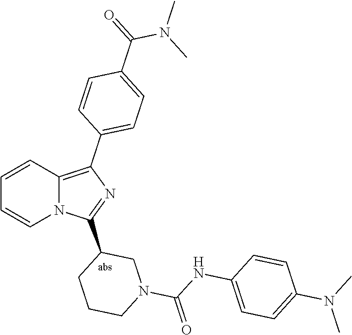 [SMILES: CN(C)C(=O)c1ccc(-c2nc([C@@H]3CCCN(C(=O)Nc4ccc(N(C)C)cc4)C3)n3ccccc23)cc1] |
| 10-47 | 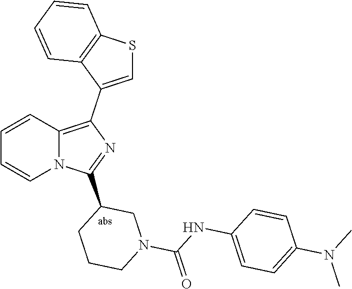 [SMILES: CN(C)c1ccc(NC(=O)N2CCC[C@@H](c3nc(-c4csc5ccccc45)c4ccccn34)C2)cc1] |
| 10-48 | 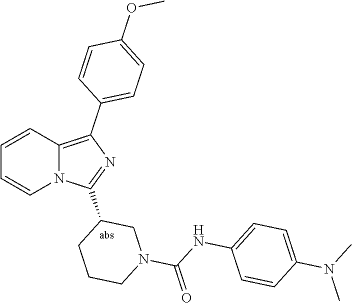 [SMILES: COc1ccc(-c2nc([C@H]3CCCN(C(=O)Nc4ccc(N(C)C)cc4)C3)n3ccccc23)cc1] |
| 10-49 | 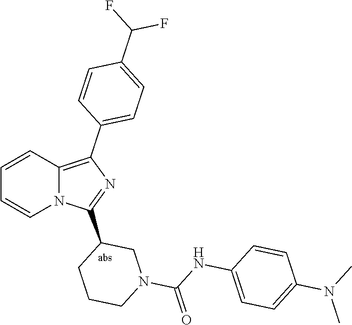 [SMILES: CN(C)c1ccc(NC(=O)N2CCC[C@@H](c3nc(-c4ccc(C(F)F)cc4)c4ccccn34)C2)cc1] |
| 10-50 | 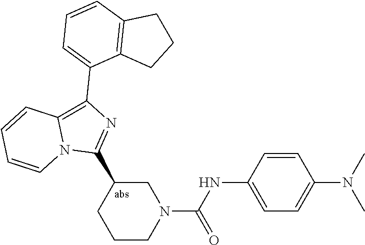 [SMILES: CN(C)c1ccc(NC(=O)N2CCC[C@@H](c3nc(-c4cccc5c4CCC5)c4ccccn34)C2)cc1] |
| 10-51 | 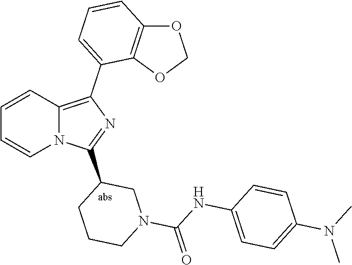 [SMILES: CN(C)c1ccc(NC(=O)N2CCC[C@@H](c3nc(-c4cccc5c4OCO5)c4ccccn34)C2)cc1] |
| 10-52 | 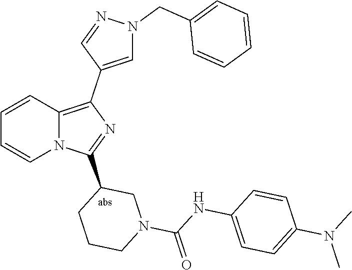 [SMILES: CN(C)c1ccc(NC(=O)N2CCC[C@@H](c3nc(-c4cnn(Cc5ccccc5)c4)c4ccccn34)C2)cc1] |
| 10-53 | 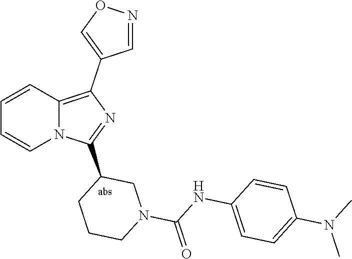 [SMILES: CN(C)c1ccc(NC(=O)N2CCC[C@@H](c3nc(-c4cnoc4)c4ccccn34)C2)cc1] |
| 10-54 | 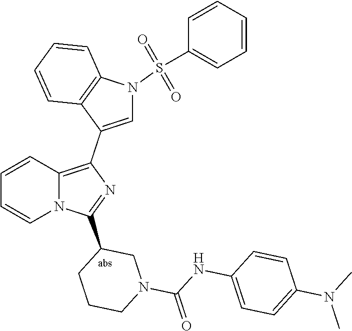 [SMILES: CN(C)c1ccc(NC(=O)N2CCC[C@@H](c3nc(-c4cn(S(=O)(=O)c5ccccc5)c5ccccc45)c4ccccn34)C2)cc1] |
| 10-55 | 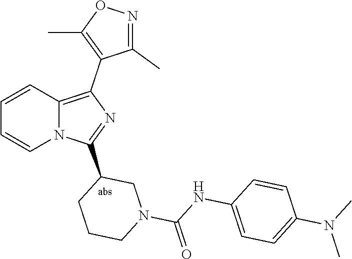 [SMILES: Cc1noc(C)c1-c1nc([C@@H]2CCCN(C(=O)Nc3ccc(N(C)C)cc3)C2)n2ccccc12] |
| 10-56 | 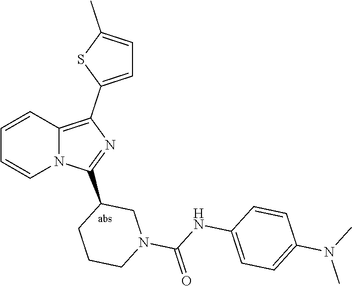 [SMILES: Cc1ccc(-c2nc([C@@H]3CCCN(C(=O)Nc4ccc(N(C)C)cc4)C3)n3ccccc23)s1] |
| 10-57 | 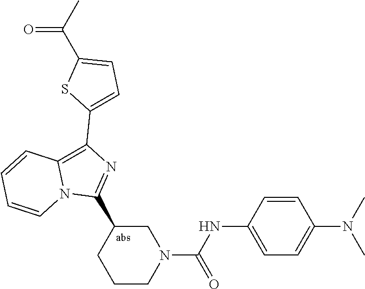 [SMILES: CC(=O)c1ccc(-c2nc([C@@H]3CCCN(C(=O)Nc4ccc(N(C)C)cc4)C3)n3ccccc23)s1] |
| 10-58 | 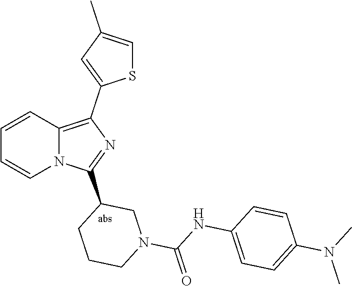 [SMILES: Cc1csc(-c2nc([C@@H]3CCCN(C(=O)Nc4ccc(N(C)C)cc4)C3)n3ccccc23)c1] |
| 10-59 | 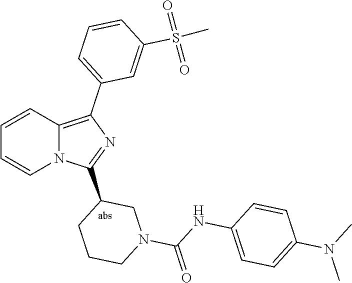 [SMILES: CN(C)c1ccc(NC(=O)N2CCC[C@@H](c3nc(-c4cccc(S(C)(=O)=O)c4)c4ccccn34)C2)cc1] |
| 10-60 | 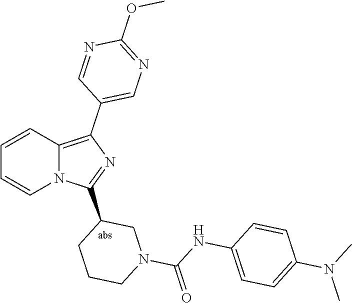 [SMILES: COc1ncc(-c2nc([C@@H]3CCCN(C(=O)Nc4ccc(N(C)C)cc4)C3)n3ccccc23)cn1] |
| 10-61 | 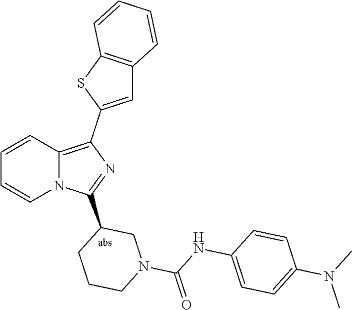 [SMILES: CN(C)c1ccc(NC(=O)N2CCC[C@@H](c3nc(-c4cc5ccccc5s4)c4ccccn34)C2)cc1] |
| 10-62 | 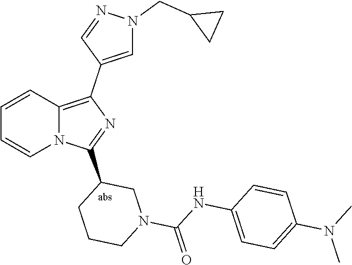 [SMILES: CN(C)c1ccc(NC(=O)N2CCC[C@@H](c3nc(-c4cnn(CC5CC5)c4)c4ccccn34)C2)cc1] |
| 10-63 | 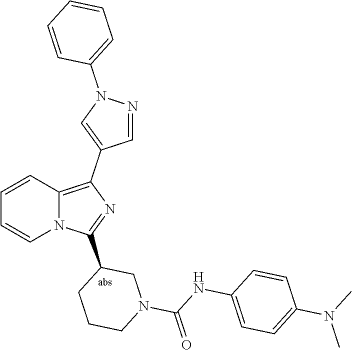 [SMILES: CN(C)c1ccc(NC(=O)N2CCC[C@@H](c3nc(-c4cnn(-c5ccccc5)c4)c4ccccn34)C2)cc1] |
| 10-64 | 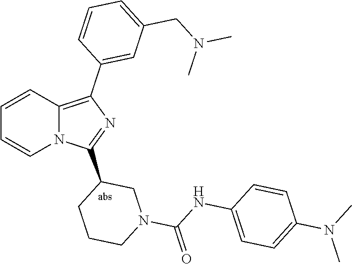 [SMILES: CN(C)Cc1cccc(-c2nc([C@@H]3CCCN(C(=O)Nc4ccc(N(C)C)cc4)C3)n3ccccc23)c1] |
| 10-65 | 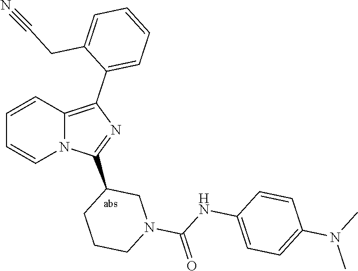 [SMILES: CN(C)c1ccc(NC(=O)N2CCC[C@@H](c3nc(-c4ccccc4CC#N)c4ccccn34)C2)cc1] |
| 10-66 | 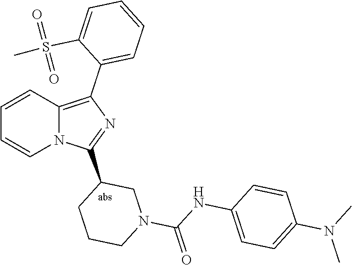 [SMILES: CN(C)c1ccc(NC(=O)N2CCC[C@@H](c3nc(-c4ccccc4S(C)(=O)=O)c4ccccn34)C2)cc1] |
| 10-67 | 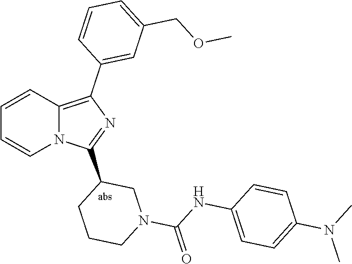 [SMILES: COCc1cccc(-c2nc([C@@H]3CCCN(C(=O)Nc4ccc(N(C)C)cc4)C3)n3ccccc23)c1] |
| 10-68 | 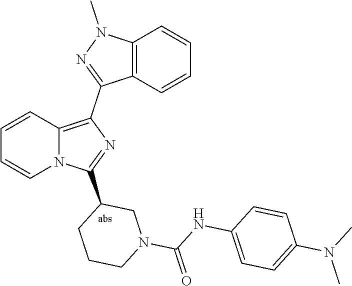 [SMILES: CN(C)c1ccc(NC(=O)N2CCC[C@@H](c3nc(-c4nn(C)c5ccccc45)c4ccccn34)C2)cc1] |
| 10-69 | 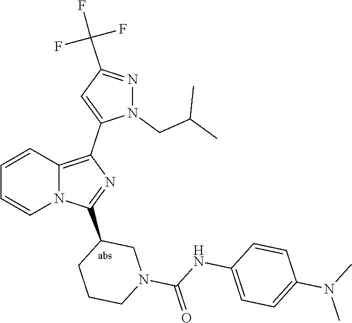 [SMILES: CC(C)Cn1nc(C(F)(F)F)cc1-c1nc([C@@H]2CCCN(C(=O)Nc3ccc(N(C)C)cc3)C2)n2ccccc12] |
| 10-70 | 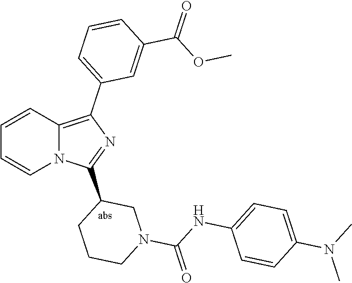 [SMILES: COC(=O)c1cccc(-c2nc([C@@H]3CCCN(C(=O)Nc4ccc(N(C)C)cc4)C3)n3ccccc23)c1] |
| 10-71 | 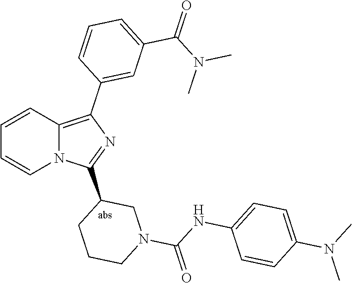 [SMILES: CN(C)C(=O)c1cccc(-c2nc([C@@H]3CCCN(C(=O)Nc4ccc(N(C)C)cc4)C3)n3ccccc23)c1] |
| 10-72 | 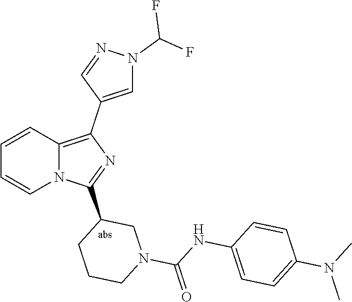 [SMILES: CN(C)c1ccc(NC(=O)N2CCC[C@@H](c3nc(-c4cnn(C(F)F)c4)c4ccccn34)C2)cc1] |
| 20-1 | 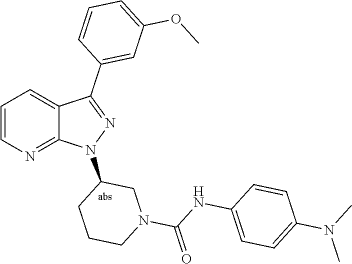 [SMILES: COc1cccc(-c2nn([C@@H]3CCCN(C(=O)Nc4ccc(N(C)C)cc4)C3)c3ncccc23)c1] |
| 20-2 | 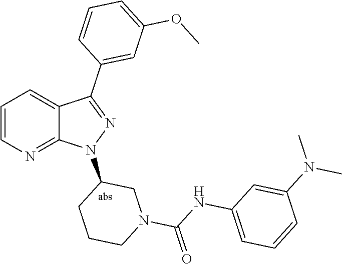 [SMILES: COc1cccc(-c2nn([C@@H]3CCCN(C(=O)Nc4cccc(N(C)C)c4)C3)c3ncccc23)c1] |
| 20-3 | 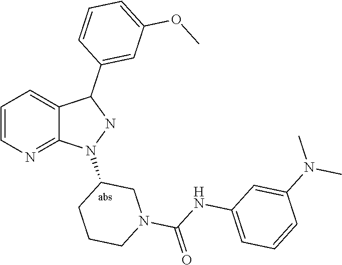 [SMILES: COc1cccc(C2NN([C@H]3CCCN(C(=O)Nc4cccc(N(C)C)c4)C3)c3ncccc32)c1] |
| 20-4 | 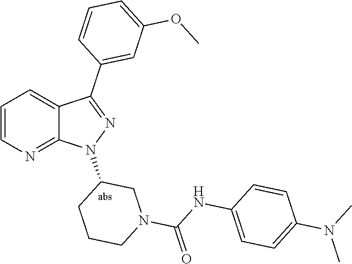 [SMILES: COc1cccc(-c2nn([C@H]3CCCN(C(=O)Nc4ccc(N(C)C)cc4)C3)c3ncccc23)c1] |
| 21-5 | 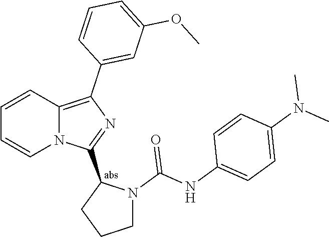 [SMILES: COc1cccc(-c2nc([C@@H]3CCCN3C(=O)Nc3ccc(N(C)C)cc3)n3ccccc23)c1] |
| 21-6 | 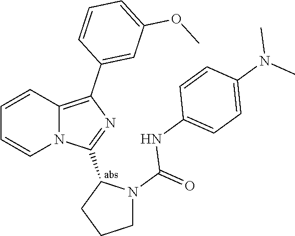 [SMILES: COc1cccc(-c2nc([C@H]3CCCN3C(=O)Nc3ccc(N(C)C)cc3)n3ccccc23)c1] |
| 23-1 | 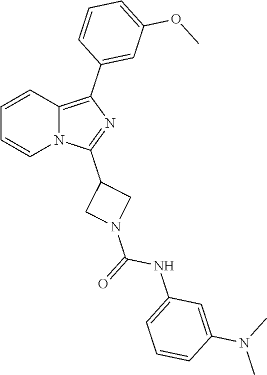 [SMILES: COc1cccc(-c2nc(C3CN(C(=O)Nc4cccc(N(C)C)c4)C3)n3ccccc23)c1] |
| 23-2 | 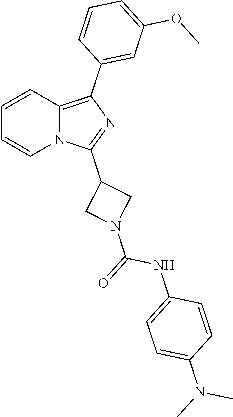 [SMILES: COc1cccc(-c2nc(C3CN(C(=O)Nc4ccc(N(C)C)cc4)C3)n3ccccc23)c1] |
| 24-1 | 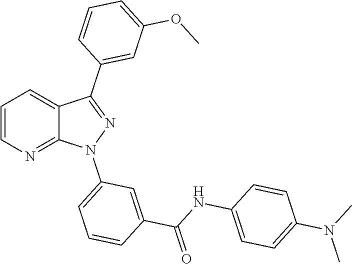 [SMILES: COc1cccc(-c2nn(-c3cccc(C(=O)Nc4ccc(N(C)C)cc4)c3)c3ncccc23)c1] |
| 24-2 | 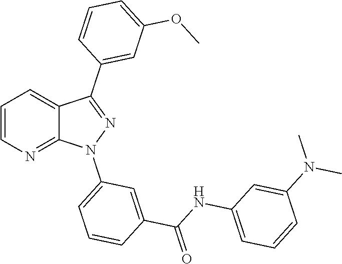 [SMILES: COc1cccc(-c2nn(-c3cccc(C(=O)Nc4cccc(N(C)C)c4)c3)c3ncccc23)c1] |
| 25-1 | 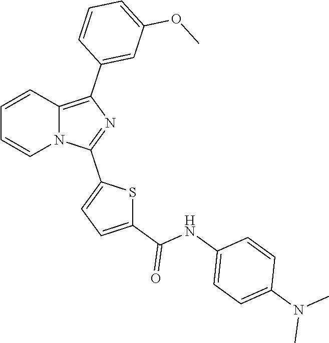 [SMILES: COc1cccc(-c2nc(-c3ccc(C(=O)Nc4ccc(N(C)C)cc4)s3)n3ccccc23)c1] |
| 26-1 | 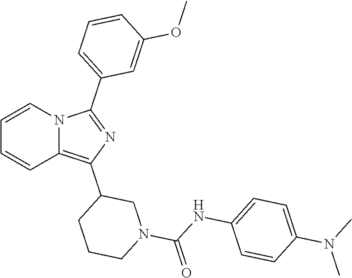 [SMILES: COc1cccc(-c2nc(C3CCCN(C(=O)Nc4ccc(N(C)C)cc4)C3)c3ccccn23)c1] |

In some embodiments, compounds having activity as modulators of the Mas-related G-protein coupled receptor D are provided, the compounds having the following structure (II):

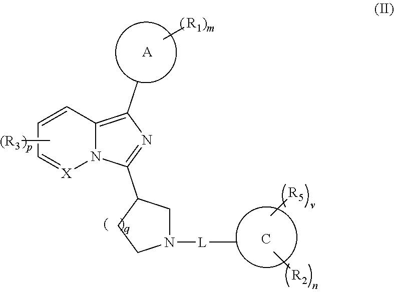

or a pharmaceutically acceptable salt, isomer, hydrate, solvate, or isotope thereof, wherein:
A is carbocyclyl or heterocyclyl, or A is

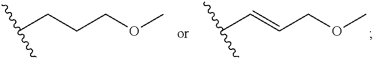

C is carbocyclyl or heterocyclyl;
L is a linker having the structure —C(O)—, —C(O)-alkyl-, —C(O)—NRa-, —C(O)—NRa-alkyl-, —S(O)₂—, or —S(O)₂-alkyl-;
X is CR₃, CH, or N;

R₁ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, alkoxycarbonyl, aminylcarbonyl, alkylsulfonyl, cycloalkylalkyl, aryl, aralkyl, or arylsulfonyl;
R₂ is hydroxyl, hydroxylalkyl, aminyl, aminylalkyl, cyanoalkyl, alkoxyalkyl, aminylcarbonyl, alkylsulfonyl, alkylsufonylalkyl, heterocyclyl, or alkylheteroaryl;
R₃ is alkyl or alkoxy;
R₅ is halo or alkyl;
Ra is H or alkyl;
m is 0, 1, or 2;
n is 1, 2, or 3;
p is 0, 1, 2, or 3;
q is 0, 1, or 2; and
v is 0 or 1.

In some embodiments, compounds of structure (II) are provided wherein A is carbocyclyl. In some embodiments, compounds of structure (II) are provided wherein A is aryl. In some embodiments, compounds of structure (II) are provided wherein A is cycloalkyl. In other embodiments, compounds of structure (II) are provided wherein A is heterocyclyl. In other embodiments, compounds of structure (II) are provided wherein A is heteroaryl. In certain embodiments, compounds of structure (II) are provided wherein A is selected from:

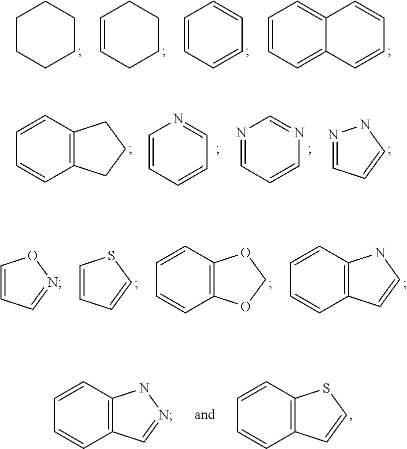

such that all valencies are satisfied. In specific embodiments, compounds of structure (II) are provided wherein A is selected from:

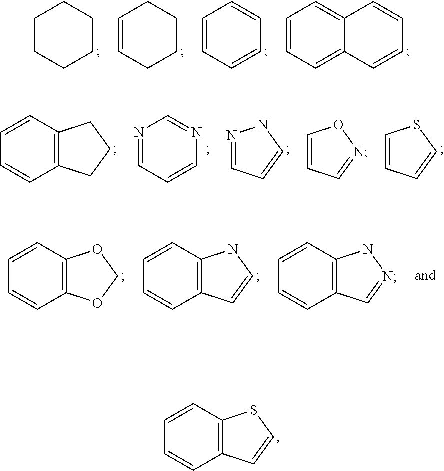

such that all valencies are satisfied. In additional embodiments, compounds of structure (II) are provided wherein A is

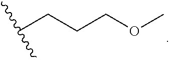 [SMILES: *CCCOC]

In further embodiments, compounds of structure (II) are provided wherein A is

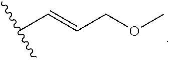 [SMILES: */C=C/COC]

In some embodiments, compounds of structure (II) are provided wherein C is carbocyclyl. In other embodiments, compounds of structure (II) are provided wherein C is cycloalkyl. In certain embodiments, compounds of structure (II) are provided wherein C is cyclopropyl, cyclobutyl, cyclopentyl, or cyclohexyl. In additional embodiments, compounds of structure (II) are provided wherein C is aryl. In specific embodiments, compounds of structure (II) are provided wherein C is phenyl. In further embodiments, compounds of structure (II) are provided wherein C is selected from:

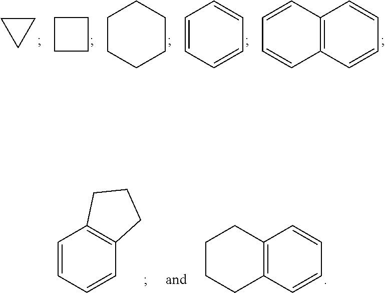

In some embodiments, compounds of structure (II) are provided wherein C is selected from:

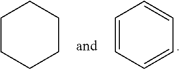 [SMILES: C1CCCCC1 ; c1ccccc1]

In additional embodiments, compounds of structure (II) are provided wherein C is heterocyclyl. In other embodiments, compounds of structure (II) are provided wherein C is saturated heterocyclyl. In yet other embodiments, compounds of structure (II) are provided wherein C is heteroaryl. In certain embodiments, compounds of structure (II) are provided wherein C is selected from:

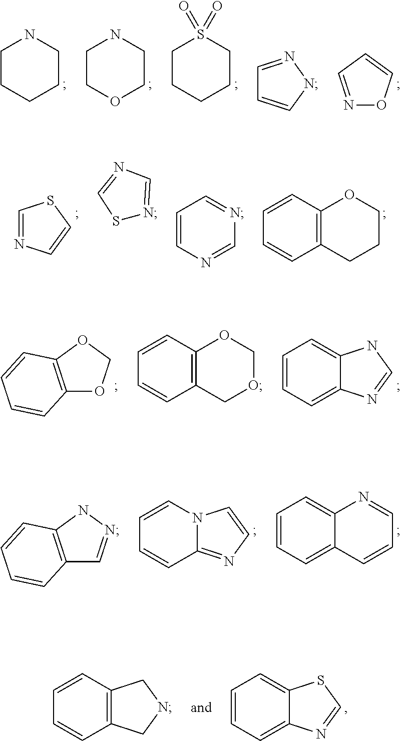

wherein all valencies are satisfied. In specific embodiments, compounds of structure (II) are provided wherein C is selected from:

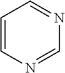

wherein all valencies are satisfied.

In some embodiments, compounds of structure (II) are provided wherein L is a linker having the structure —C(O)—. In other embodiments, compounds of structure (II) are provided wherein L is a linker having the structure —C(O)-alkyl-. In additional embodiments, compounds of structure (II) are provided wherein L is a linker having the structure —C(O)—NRa-. In yet other embodiments, compounds of structure (II) are provided wherein L is a linker having the structure —C(O)—NRa-alkyl-. In further embodiments, compounds of structure (II) are provided wherein L is a linker having the structure —S(O)₂—. In some embodiments, compounds of structure (II) are provided wherein L is a linker having the structure —S(O)₂-alkyl-.

In some embodiments, compounds of structure (II) are provided wherein X is CR₃. In other embodiments, compounds of structure (II) are provided wherein X is CH. In additional embodiments, compounds of structure (II) are provided wherein X is N.

In some embodiments, compounds of structure (II) are provided wherein R₁ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, alkoxycarbonyl, aminylcarbonyl, or alkylsulfonyl. In other embodiments, compounds of structure (II) are provided wherein R₁ is cycloalkylalkyl, aryl, aralkyl, or arylsulfonyl. In certain embodiments, compounds of structure (II) are provided wherein R₁ is selected from:

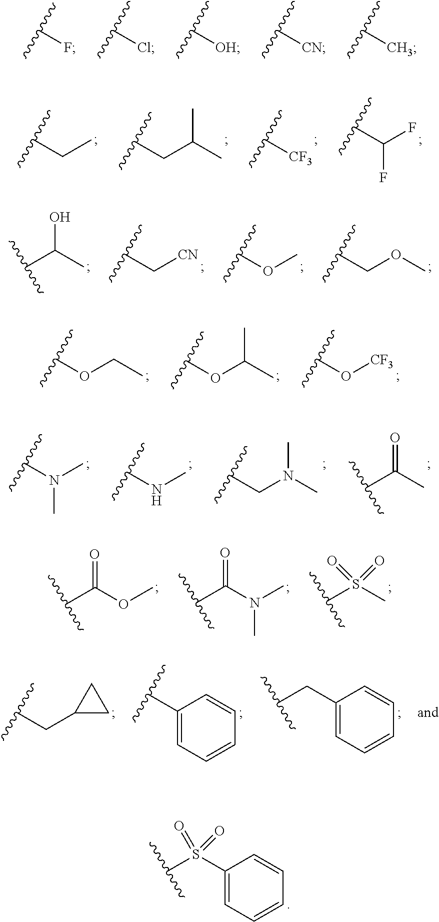

In some embodiments, compounds of structure (II) are provided wherein R₂ is hydroxyl, hydroxylalkyl, aminyl, aminylalkyl, cyanoalkyl, alkoxyalkyl, aminylcarbonyl, alkylsulfonyl, or alkylsufonylalkyl. In some embodiments, compounds of structure (II) are provided wherein R₂ is hydroxyl, hydroxylalkyl, aminyl, aminylalkyl, cyanoalkyl, alkoxyalkyl, aminylcarbonyl, or alkylsulfonyl. In other embodiments, compounds of structure (II) are provided wherein R₂ is heterocyclyl or alkylheteroaryl. In certain embodiments, compounds of structure (II) are provided wherein R₂ is selected from:

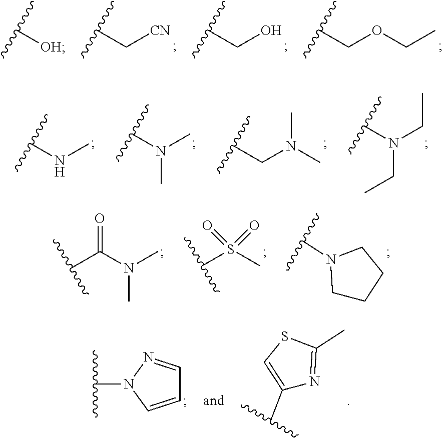

In some embodiments, compounds of structure (II) are provided wherein R₃ is alkyl. In other embodiments, compounds of structure (II) are provided wherein R₃ is alkoxy.

In some embodiments, compounds of structure (II) are provided wherein R₅ is halo. In other embodiments, compounds of structure (II) are provided wherein R₅ is alkyl.

In some embodiments, compounds of structure (II) are provided wherein Ra is H. In other embodiments, compounds of structure (II) are provided wherein Ra is alkyl.

In some embodiments, compounds of structure (II) are provided wherein m is 0. In other embodiments, compounds of structure (II) are provided wherein m is 1. In additional embodiments, compounds of structure (II) are provided wherein m is 2.

In other embodiments, compounds of structure (II) are provided wherein n is 1. In additional embodiments, compounds of structure (II) are provided wherein n is 2. In further embodiments, compounds of structure (II) are provided wherein n is 3.

In some embodiments, compounds of structure (II) are provided wherein p is 0. In other embodiments, compounds of structure (II) are provided wherein p is 1. In additional embodiments, compounds of structure (II) are provided wherein p is 2. In further embodiments, compounds of structure (II) are provided wherein p is 3.

In some embodiments, compounds of structure (II) are provided wherein q is 0. In other embodiments, compounds of structure (II) are provided wherein q is 1. In additional embodiments, compounds of structure (II) are provided wherein q is 2.

In some embodiments, compounds of structure (II) are provided wherein v is 0. In other embodiments, compounds of structure (II) are provided wherein v is 1.

Representative compounds of structure (II) include any one of the compounds listed in Table II below, as well as a pharmaceutically acceptable isomer, racemate, hydrate, solvate, isotope, or salt thereof.

TABLE II
Compounds of Structure II

| Cpd. No. | Structure |
|---|---|
| 1-1 | 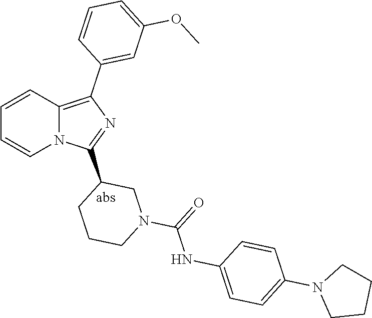 [SMILES: COc1cccc(-c2nc([C@@H]3CCCN(C(=O)Nc4ccc(N5CCCC5)cc4)C3)n3ccccc23)c1] |
| 1-2 | 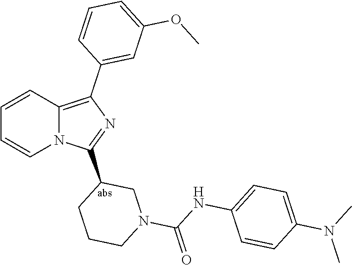 [SMILES: COc1cccc(-c2nc([C@@H]3CCCN(C(=O)Nc4ccc(N(C)C)cc4)C3)n3ccccc23)c1] |
| 1-5 | 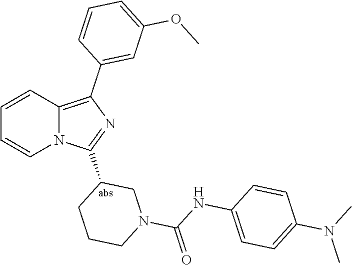 [SMILES: COc1cccc(-c2nc([C@H]3CCCN(C(=O)Nc4ccc(N(C)C)cc4)C3)n3ccccc23)c1] |
| 1-6 | 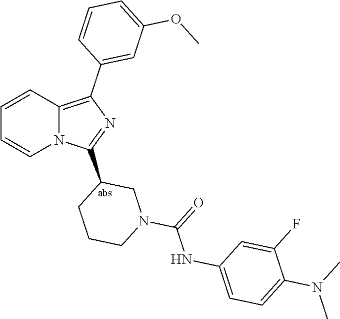 [SMILES: COc1cccc(-c2nc([C@@H]3CCCN(C(=O)Nc4ccc(N(C)C)c(F)c4)C3)n3ccccc23)c1] |
| 1-7 | 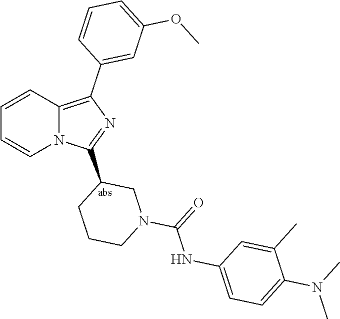 [SMILES: COc1cccc(-c2nc([C@@H]3CCCN(C(=O)Nc4ccc(N(C)C)c(C)c4)C3)n3ccccc23)c1] |
| 1-8 | 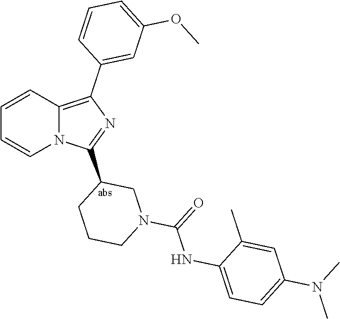 [SMILES: COc1cccc(-c2nc([C@@H]3CCCN(C(=O)Nc4ccc(N(C)C)cc4C)C3)n3ccccc23)c1] |
| 1-19 | 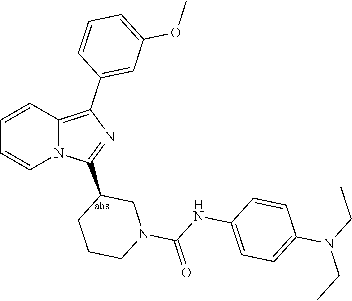 [SMILES: CCN(CC)c1ccc(NC(=O)N2CCC[C@@H](c3nc(-c4cccc(OC)c4)c4ccccn34)C2)cc1] |
| 1-20 | 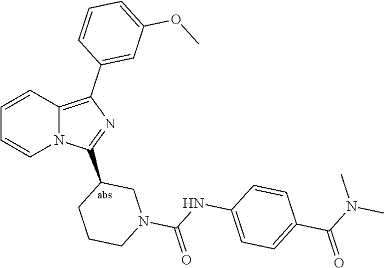 [SMILES: COc1cccc(-c2nc([C@@H]3CCCN(C(=O)Nc4ccc(C(=O)N(C)C)cc4)C3)n3ccccc23)c1] |
| 1-21 | 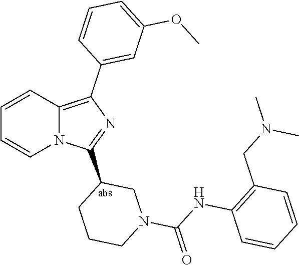 [SMILES: COc1cccc(-c2nc([C@@H]3CCCN(C(=O)Nc4ccccc4CN(C)C)C3)n3ccccc23)c1] |
| 1-26 | 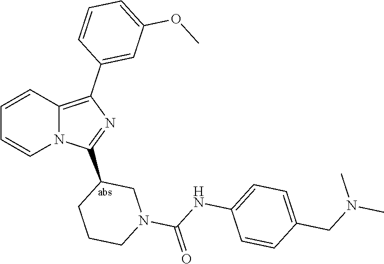 [SMILES: COc1cccc(-c2nc([C@@H]3CCCN(C(=O)Nc4ccc(CN(C)C)cc4)C3)n3ccccc23)c1] |
| 3-24 | 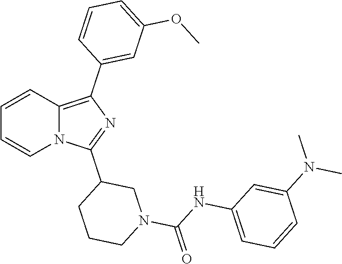 [SMILES: COc1cccc(-c2nc(C3CCCN(C(=O)Nc4cccc(N(C)C)c4)C3)n3ccccc23)c1] |
| 3-25 | 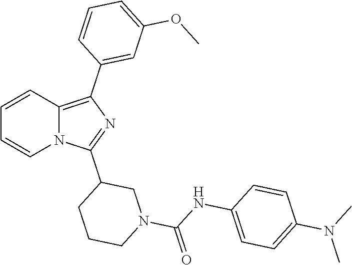 [SMILES: COc1cccc(-c2nc(C3CCCN(C(=O)Nc4ccc(N(C)C)cc4)C3)n3ccccc23)c1] |
| 4-3 | 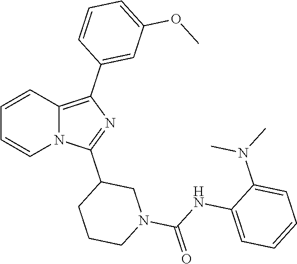 [SMILES: COc1cccc(-c2nc(C3CCCN(C(=O)Nc4ccccc4N(C)C)C3)n3ccccc23)c1] |
| 5-7 | 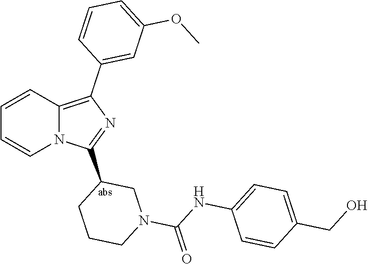 [SMILES: COc1cccc(-c2nc([C@@H]3CCCN(C(=O)Nc4ccc(CO)cc4)C3)n3ccccc23)c1] |
| 5-16 | 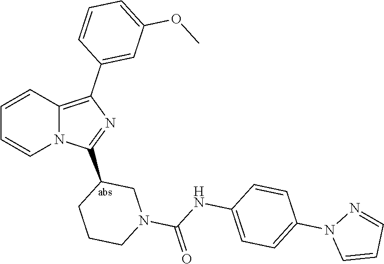 [SMILES: COc1cccc(-c2nc([C@@H]3CCCN(C(=O)Nc4ccc(-n5cccn5)cc4)C3)n3ccccc23)c1] |
| 5-17 | 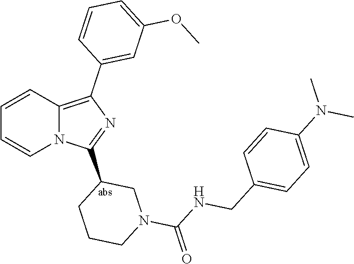 [SMILES: COc1cccc(-c2nc([C@@H]3CCCN(C(=O)NCc4ccc(N(C)C)cc4)C3)n3ccccc23)c1] |
| 5-18 | 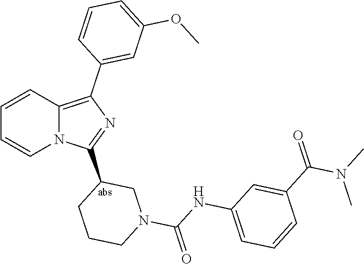 [SMILES: COc1cccc(-c2nc([C@@H]3CCCN(C(=O)Nc4cccc(C(=O)N(C)C)c4)C3)n3ccccc23)c1] |
| 5-19 | 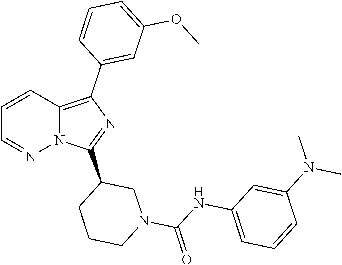 [SMILES: COc1cccc(-c2nc([C@@H]3CCCN(C(=O)Nc4cccc(N(C)C)c4)C3)n3ncccc23)c1] |
| 5-20 | 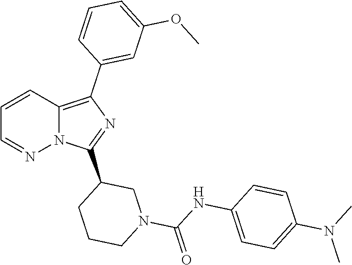 [SMILES: COc1cccc(-c2nc([C@@H]3CCCN(C(=O)Nc4ccc(N(C)C)cc4)C3)n3ncccc23)c1] |
| 5-28 | 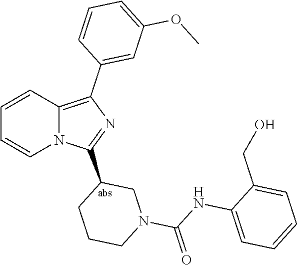 [SMILES: COc1cccc(-c2nc([C@@H]3CCCN(C(=O)Nc4ccccc4CO)C3)n3ccccc23)c1] |
| 5-31 | 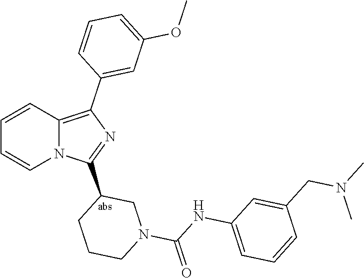 [SMILES: COc1cccc(-c2nc([C@@H]3CCCN(C(=O)Nc4cccc(CN(C)C)c4)C3)n3ccccc23)c1] |
| 5-33 | 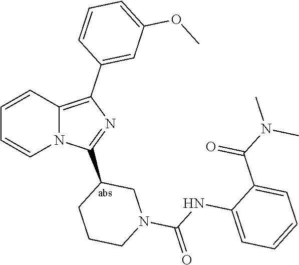 [SMILES: COc1cccc(-c2nc([C@@H]3CCCN(C(=O)Nc4ccccc4C(=O)N(C)C)C3)n3ccccc23)c1] |
| 5-36 | 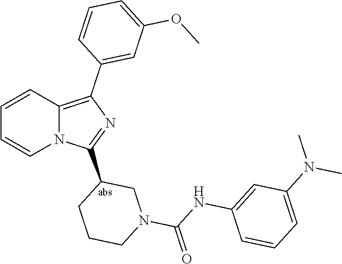 [SMILES: COc1cccc(-c2nc([C@@H]3CCCN(C(=O)Nc4cccc(N(C)C)c4)C3)n3ccccc23)c1] |
| 5-37 | 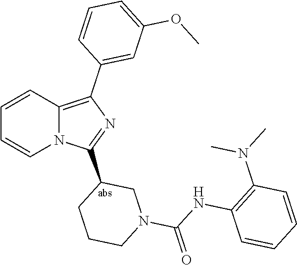 [SMILES: COc1cccc(-c2nc([C@@H]3CCCN(C(=O)Nc4ccccc4N(C)C)C3)n3ccccc23)c1] |
| 5-49 | 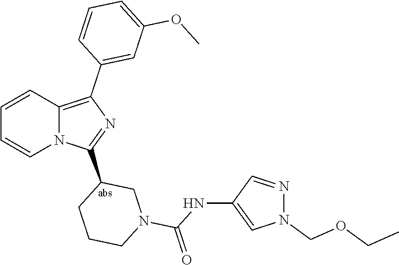 [SMILES: CCOCn1cc(NC(=O)N2CCC[C@@H](c3nc(-c4cccc(OC)c4)c4ccccn34)C2)cn1] |
| 5-56 | 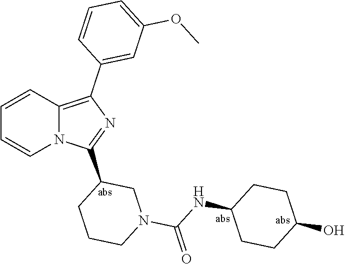 [SMILES: COc1cccc(-c2nc([C@@H]3CCCN(C(=O)N[C@H]4CC[C@@H](O)CC4)C3)n3ccccc23)c1] |
| 5-61 | 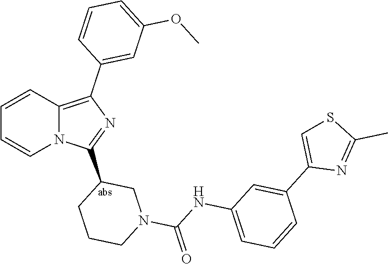 [SMILES: COc1cccc(-c2nc([C@@H]3CCCN(C(=O)Nc4cccc(-c5csc(C)n5)c4)C3)n3ccccc23)c1] |
| 5-63 | 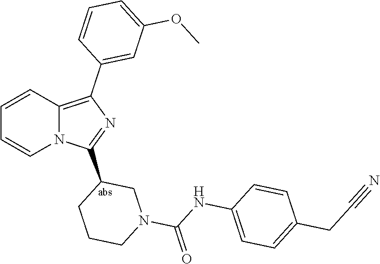 [SMILES: COc1cccc(-c2nc([C@@H]3CCCN(C(=O)Nc4ccc(CC#N)cc4)C3)n3ccccc23)c1] |
| 5-70 | 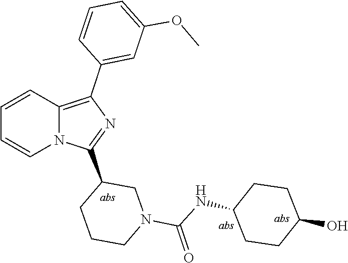 [SMILES: COc1cccc(-c2nc([C@@H]3CCCN(C(=O)N[C@H]4CC[C@H](O)CC4)C3)n3ccccc23)c1] |
| 5-72 | 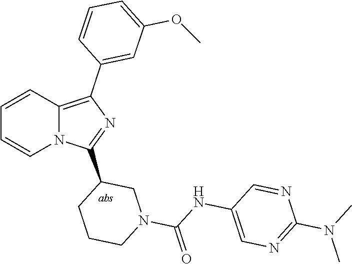 [SMILES: COc1cccc(-c2nc([C@@H]3CCCN(C(=O)Nc4cnc(N(C)C)nc4)C3)n3ccccc23)c1] |
| 6-1 | 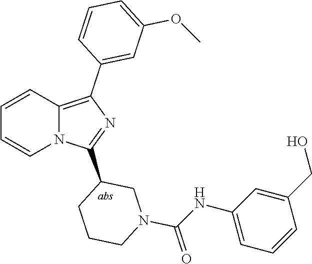 [SMILES: COc1cccc(-c2nc([C@@H]3CCCN(C(=O)Nc4cccc(CO)c4)C3)n3ccccc23)c1] |
| 7-1 | 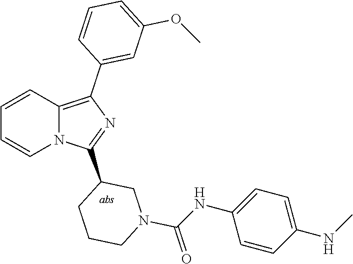 [SMILES: CNc1ccc(NC(=O)N2CCC[C@@H](c3nc(-c4cccc(OC)c4)c4ccccn34)C2)cc1] |
| 10-1 | 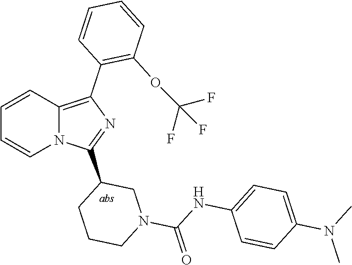 [SMILES: CN(C)c1ccc(NC(=O)N2CCC[C@@H](c3nc(-c4ccccc4OC(F)(F)F)c4ccccn34)C2)cc1] |
| 10-2 | 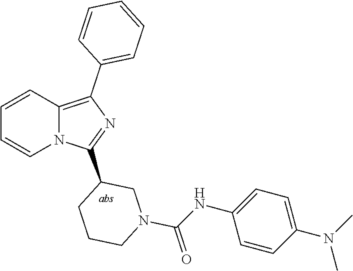 [SMILES: CN(C)c1ccc(NC(=O)N2CCC[C@@H](c3nc(-c4ccccc4)c4ccccn34)C2)cc1] |
| 10-3 | 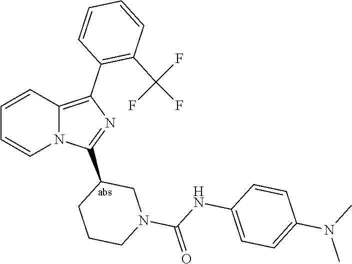 [SMILES: CN(C)c1ccc(NC(=O)N2CCC[C@@H](c3nc(-c4ccccc4C(F)(F)F)c4ccccn34)C2)cc1] |
| 10-4 | 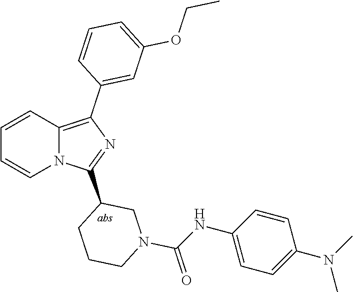 [SMILES: CCOc1cccc(-c2nc([C@@H]3CCCN(C(=O)Nc4ccc(N(C)C)cc4)C3)n3ccccc23)c1] |
| 10-5 | 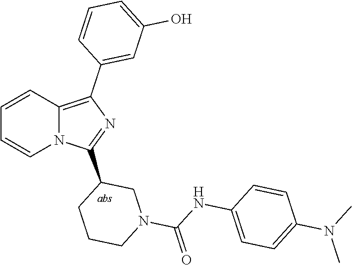 [SMILES: CN(C)c1ccc(NC(=O)N2CCC[C@@H](c3nc(-c4cccc(O)c4)c4ccccn34)C2)cc1] |
| 10-6 | 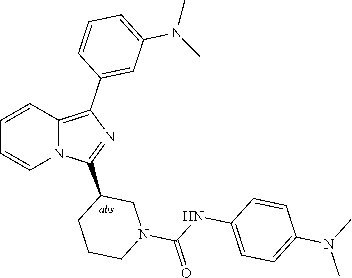 [SMILES: CN(C)c1ccc(NC(=O)N2CCC[C@@H](c3nc(-c4cccc(N(C)C)c4)c4ccccn34)C2)cc1] |
| 10-7 | 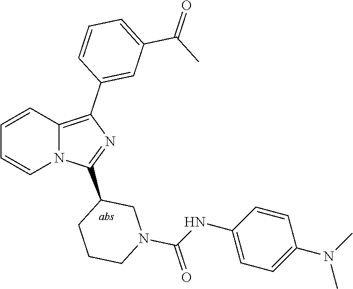 [SMILES: CC(=O)c1cccc(-c2nc([C@@H]3CCCN(C(=O)Nc4ccc(N(C)C)cc4)C3)n3ccccc23)c1] |
| 10-8 | 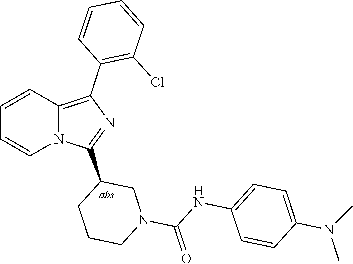 [SMILES: CN(C)c1ccc(NC(=O)N2CCC[C@@H](c3nc(-c4ccccc4Cl)c4ccccn34)C2)cc1] |
| 10-9 | 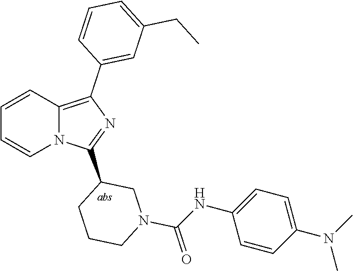 [SMILES: CCc1cccc(-c2nc([C@@H]3CCCN(C(=O)Nc4ccc(N(C)C)cc4)C3)n3ccccc23)c1] |
| 10-10 | 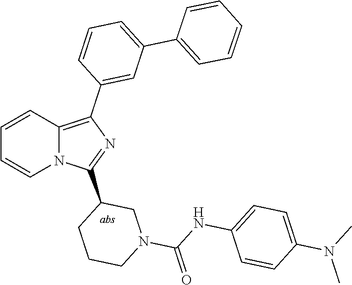 [SMILES: CN(C)c1ccc(NC(=O)N2CCC[C@@H](c3nc(-c4cccc(-c5ccccc5)c4)c4ccccn34)C2)cc1] |
| 10-11 | 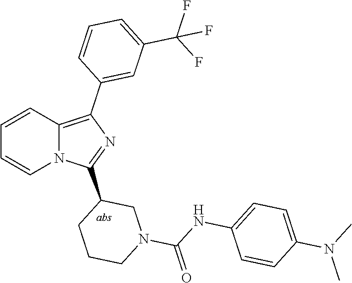 [SMILES: CN(C)c1ccc(NC(=O)N2CCC[C@@H](c3nc(-c4cccc(C(F)(F)F)c4)c4ccccn34)C2)cc1] |
| 10-12 | 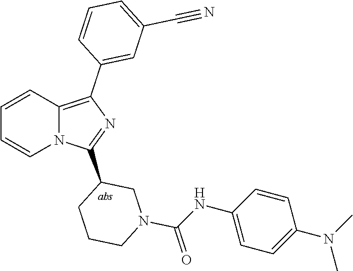 [SMILES: CN(C)c1ccc(NC(=O)N2CCC[C@@H](c3nc(-c4cccc(C#N)c4)c4ccccn34)C2)cc1] |
| 10-13 | 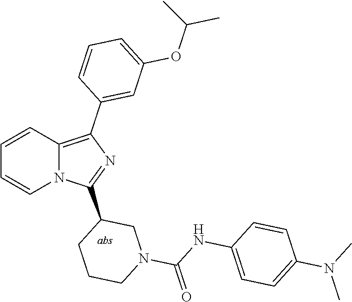 [SMILES: CC(C)Oc1cccc(-c2nc([C@@H]3CCCN(C(=O)Nc4ccc(N(C)C)cc4)C3)n3ccccc23)c1] |
| 10-14 | 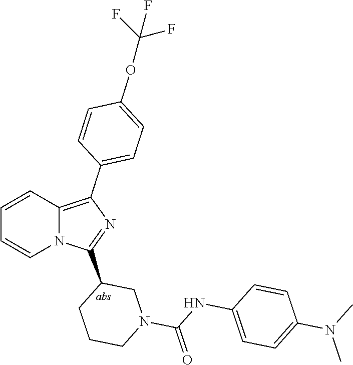 [SMILES: CN(C)c1ccc(NC(=O)N2CCC[C@@H](c3nc(-c4ccc(OC(F)(F)F)cc4)c4ccccn34)C2)cc1] |
| 10-15 | 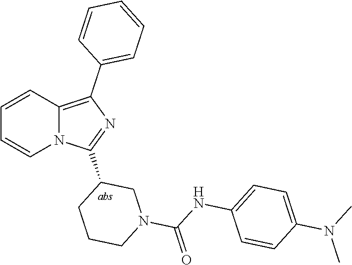 [SMILES: CN(C)c1ccc(NC(=O)N2CCC[C@H](c3nc(-c4ccccc4)c4ccccn34)C2)cc1] |
| 10-16 | 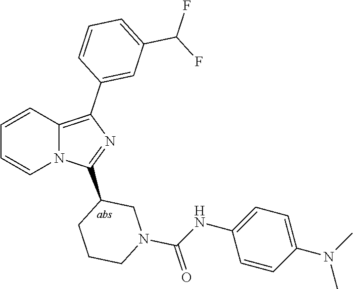 [SMILES: CN(C)c1ccc(NC(=O)N2CCC[C@@H](c3nc(-c4cccc(C(F)F)c4)c4ccccn34)C2)cc1] |
| 10-17 | 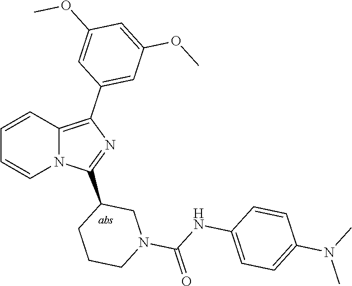 [SMILES: COc1cc(OC)cc(-c2nc([C@@H]3CCCN(C(=O)Nc4ccc(N(C)C)cc4)C3)n3ccccc23)c1] |
| 10-18 | 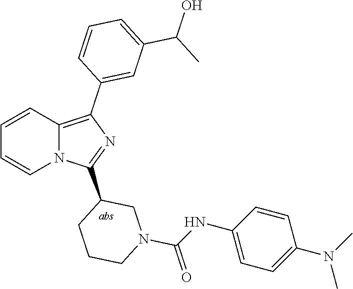 [SMILES: CC(O)c1cccc(-c2nc([C@@H]3CCCN(C(=O)Nc4ccc(N(C)C)cc4)C3)n3ccccc23)c1] |
| 10-19 | 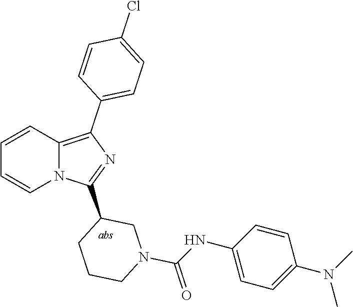 [SMILES: CN(C)c1ccc(NC(=O)N2CCC[C@@H](c3nc(-c4ccc(Cl)cc4)c4ccccn34)C2)cc1] |
| 10-20 | 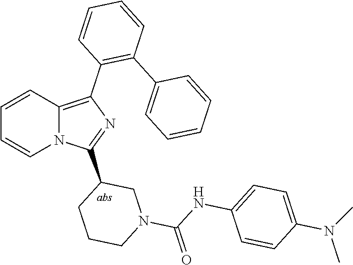 [SMILES: CN(C)c1ccc(NC(=O)N2CCC[C@@H](c3nc(-c4ccccc4-c4ccccc4)c4ccccn34)C2)cc1] |
| 10-21 | 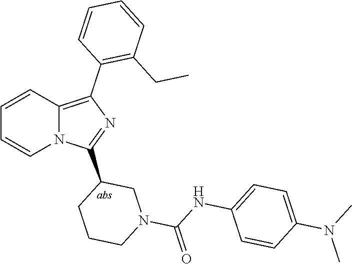 [SMILES: CCc1ccccc1-c1nc([C@@H]2CCCN(C(=O)Nc3ccc(N(C)C)cc3)C2)n2ccccc12] |
| 10-22 | 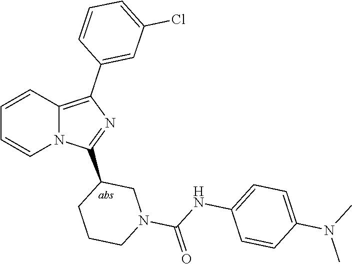 [SMILES: CN(C)c1ccc(NC(=O)N2CCC[C@@H](c3nc(-c4cccc(Cl)c4)c4ccccn34)C2)cc1] |
| 10-23 | 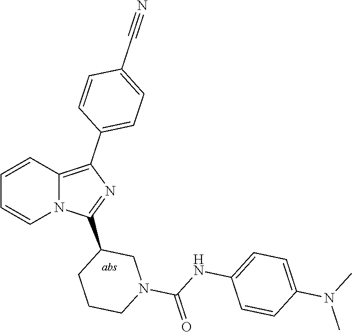 [SMILES: CN(C)c1ccc(NC(=O)N2CCC[C@@H](c3nc(-c4ccc(C#N)cc4)c4ccccn34)C2)cc1] |
| 10-24 | 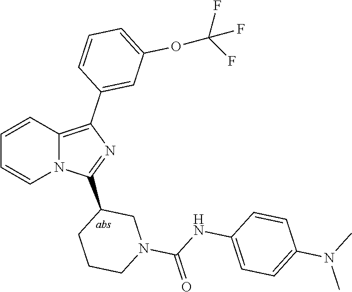 [SMILES: CN(C)c1ccc(NC(=O)N2CCC[C@@H](c3nc(-c4cccc(OC(F)(F)F)c4)c4ccccn34)C2)cc1] |
| 10-26 | 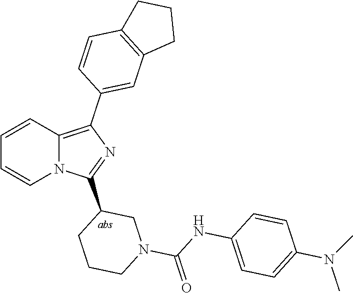 [SMILES: CN(C)c1ccc(NC(=O)N2CCC[C@@H](c3nc(-c4ccc5c(c4)CCC5)c4ccccn34)C2)cc1] |
| 10-27 | 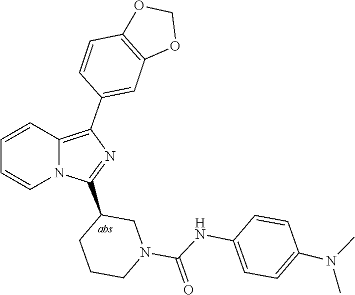 [SMILES: CN(C)c1ccc(NC(=O)N2CCC[C@@H](c3nc(-c4ccc5c(c4)OCO5)c4ccccn34)C2)cc1] |
| 10-28 | 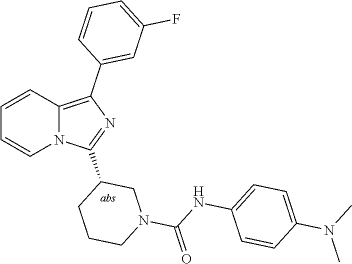 [SMILES: CN(C)c1ccc(NC(=O)N2CCC[C@H](c3nc(-c4cccc(F)c4)c4ccccn34)C2)cc1] |
| 10-29 | 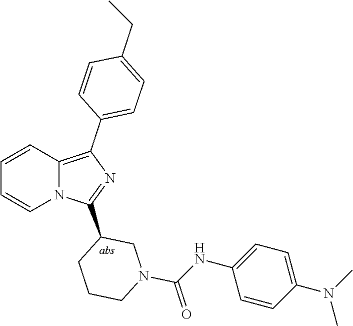 [SMILES: CCc1ccc(-c2nc([C@@H]3CCCN(C(=O)Nc4ccc(N(C)C)cc4)C3)n3ccccc23)cc1] |
| 10-30 | 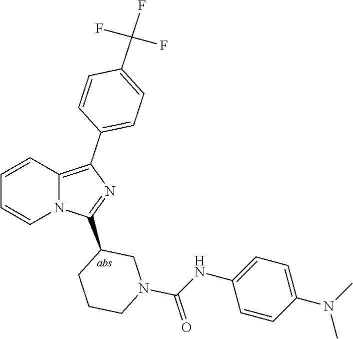 [SMILES: CN(C)c1ccc(NC(=O)N2CCC[C@@H](c3nc(-c4ccc(C(F)(F)F)cc4)c4ccccn34)C2)cc1] |
| 10-31 | 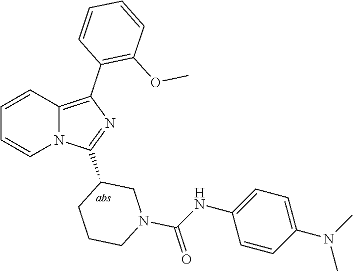 [SMILES: COc1ccccc1-c1nc([C@H]2CCCN(C(=O)Nc3ccc(N(C)C)cc3)C2)n2ccccc12] |
| 10-32 | 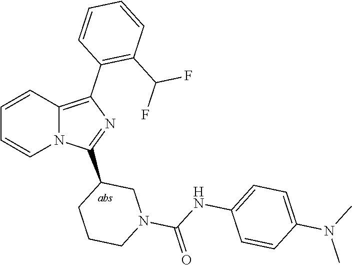 [SMILES: CN(C)c1ccc(NC(=O)N2CCC[C@@H](c3nc(-c4ccccc4C(F)F)c4ccccn34)C2)cc1] |
| 10-33 | 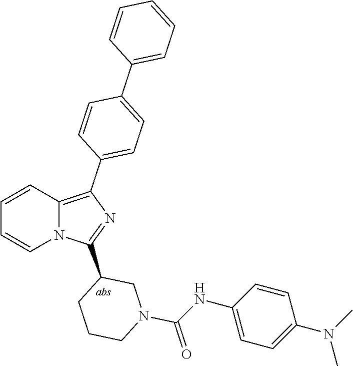 [SMILES: CN(C)c1ccc(NC(=O)N2CCC[C@@H](c3nc(-c4ccc(-c5ccccc5)cc4)c4ccccn34)C2)cc1] |
| 10-34 | 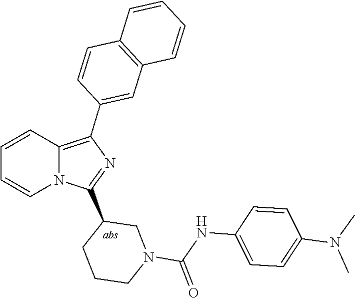 [SMILES: CN(C)c1ccc(NC(=O)N2CCC[C@@H](c3nc(-c4ccc5ccccc5c4)c4ccccn34)C2)cc1] |
| 10-35 | 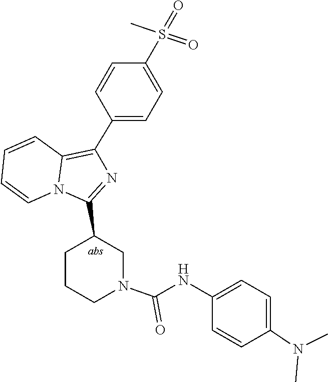 [SMILES: CN(C)c1ccc(NC(=O)N2CCC[C@@H](c3nc(-c4ccc(S(C)(=O)=O)cc4)c4ccccn34)C2)cc1] |
| 10-36 | 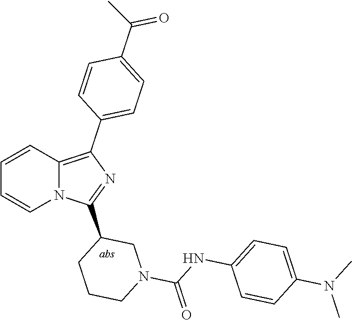 [SMILES: CC(=O)c1ccc(-c2nc([C@@H]3CCCN(C(=O)Nc4ccc(N(C)C)cc4)C3)n3ccccc23)cc1] |
| 10-37 | 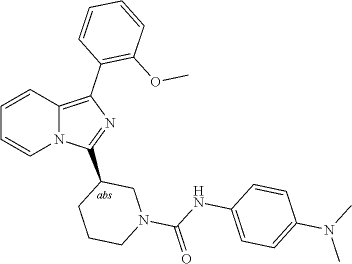 [SMILES: COc1ccccc1-c1nc([C@@H]2CCCN(C(=O)Nc3ccc(N(C)C)cc3)C2)n2ccccc12] |
| 10-39 | 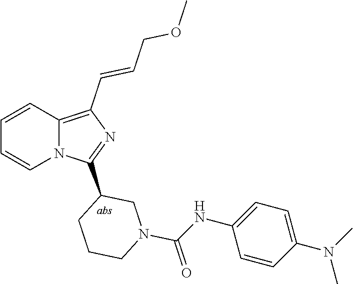 [SMILES: COC/C=C/c1nc([C@@H]2CCCN(C(=O)Nc3ccc(N(C)C)cc3)C2)n2ccccc12] |
| 10-40 | 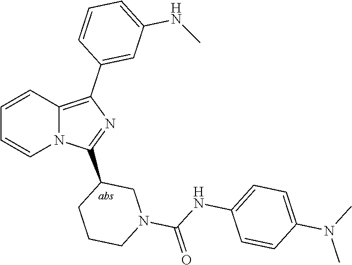 [SMILES: CNc1cccc(-c2nc([C@@H]3CCCN(C(=O)Nc4ccc(N(C)C)cc4)C3)n3ccccc23)c1] |
| 10-41 | 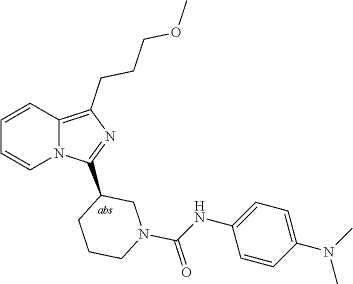 [SMILES: COCCCc1nc([C@@H]2CCCN(C(=O)Nc3ccc(N(C)C)cc3)C2)n2ccccc12] |
| 10-42 | 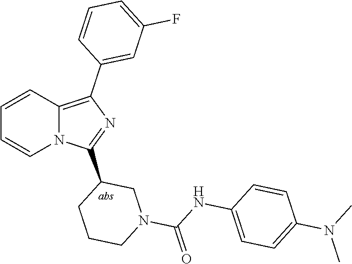 [SMILES: CN(C)c1ccc(NC(=O)N2CCC[C@@H](c3nc(-c4cccc(F)c4)c4ccccn34)C2)cc1] |
| 10-43 | 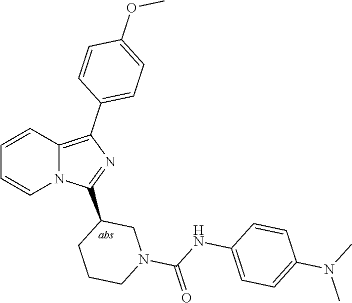 [SMILES: COc1ccc(-c2nc([C@@H]3CCCN(C(=O)Nc4ccc(N(C)C)cc4)C3)n3ccccc23)cc1] |
| 10-44 | 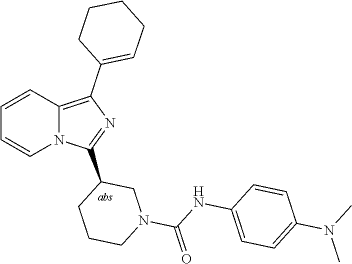 [SMILES: CN(C)c1ccc(NC(=O)N2CCC[C@@H](c3nc(C4=CCCCC4)c4ccccn34)C2)cc1] |
| 10-45 | 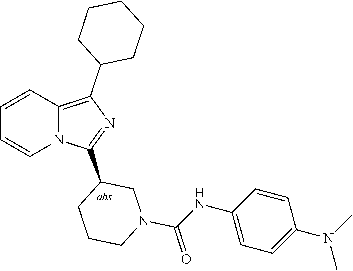 [SMILES: CN(C)c1ccc(NC(=O)N2CCC[C@@H](c3nc(C4CCCCC4)c4ccccn34)C2)cc1] |
| 10-46 | 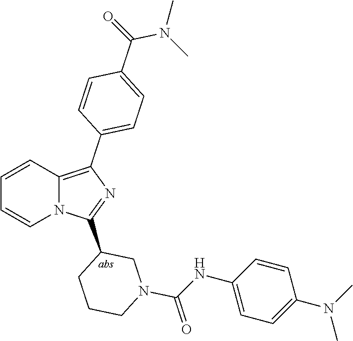 [SMILES: CN(C)C(=O)c1ccc(-c2nc([C@@H]3CCCN(C(=O)Nc4ccc(N(C)C)cc4)C3)n3ccccc23)cc1] |
| 10-47 | 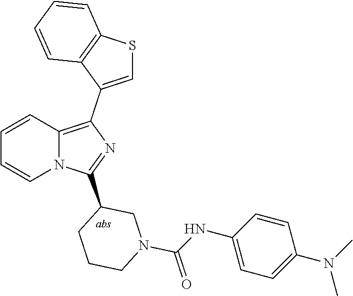 [SMILES: CN(C)c1ccc(NC(=O)N2CCC[C@@H](c3nc(-c4csc5ccccc45)c4ccccn34)C2)cc1] |
| 10-48 | 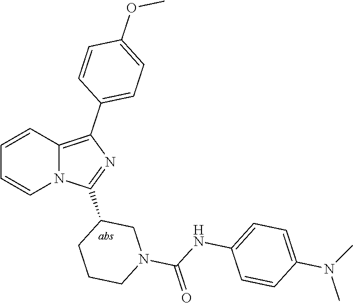 [SMILES: COc1ccc(-c2nc([C@H]3CCCN(C(=O)Nc4ccc(N(C)C)cc4)C3)n3ccccc23)cc1] |
| 10-49 | 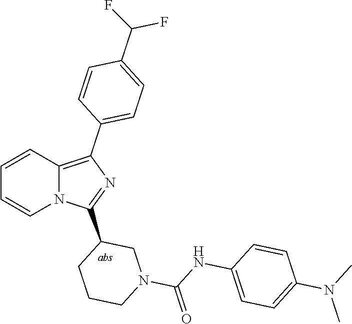 [SMILES: CN(C)c1ccc(NC(=O)N2CCC[C@@H](c3nc(-c4ccc(C(F)F)cc4)c4ccccn34)C2)cc1] |
| 10-50 | 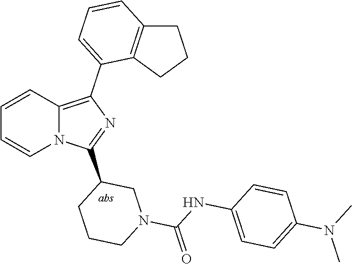 [SMILES: CN(C)c1ccc(NC(=O)N2CCC[C@@H](c3nc(-c4cccc5c4CCC5)c4ccccn34)C2)cc1] |
| 10-51 | 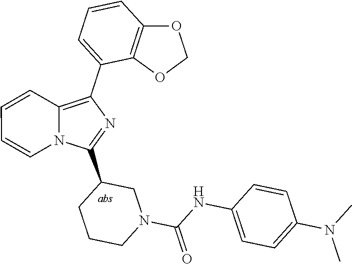 [SMILES: CN(C)c1ccc(NC(=O)N2CCC[C@@H](c3nc(-c4cccc5c4OCO5)c4ccccn34)C2)cc1] |
| 10-52 | 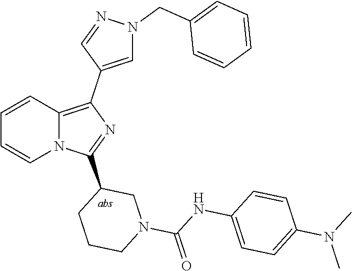 [SMILES: CN(C)c1ccc(NC(=O)N2CCC[C@@H](c3nc(-c4cnn(Cc5ccccc5)c4)c4ccccn34)C2)cc1] |
| 10-53 | 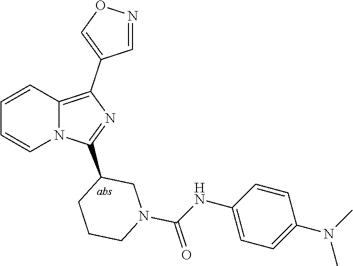 [SMILES: CN(C)c1ccc(NC(=O)N2CCC[C@@H](c3nc(-c4cnoc4)c4ccccn34)C2)cc1] |
| 10-54 | 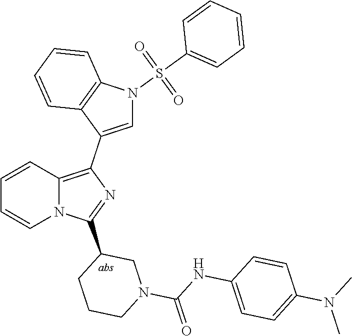 [SMILES: CN(C)c1ccc(NC(=O)N2CCC[C@@H](c3nc(-c4cn(S(=O)(=O)c5ccccc5)c5ccccc45)c4ccccn34)C2)cc1] |
| 10-55 | 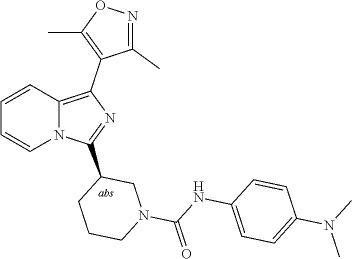 [SMILES: Cc1noc(C)c1-c1nc([C@@H]2CCCN(C(=O)Nc3ccc(N(C)C)cc3)C2)n2ccccc12] |
| 10-56 | 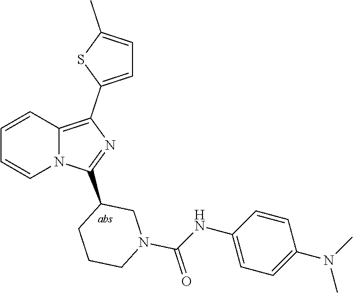 [SMILES: Cc1ccc(-c2nc([C@@H]3CCCN(C(=O)Nc4ccc(N(C)C)cc4)C3)n3ccccc23)s1] |
| 10-57 | 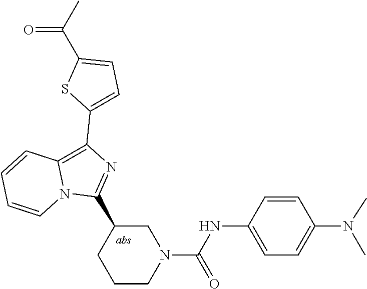 [SMILES: CC(=O)c1ccc(-c2nc([C@@H]3CCCN(C(=O)Nc4ccc(N(C)C)cc4)C3)n3ccccc23)s1] |
| 10-58 | 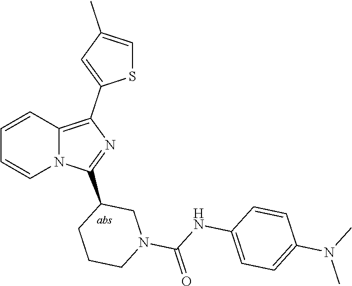 [SMILES: Cc1csc(-c2nc([C@@H]3CCCN(C(=O)Nc4ccc(N(C)C)cc4)C3)n3ccccc23)c1] |
| 10-59 | 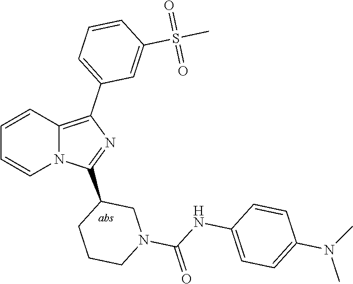 [SMILES: CN(C)c1ccc(NC(=O)N2CCC[C@@H](c3nc(-c4cccc(S(C)(=O)=O)c4)c4ccccn34)C2)cc1] |
| 10-60 | 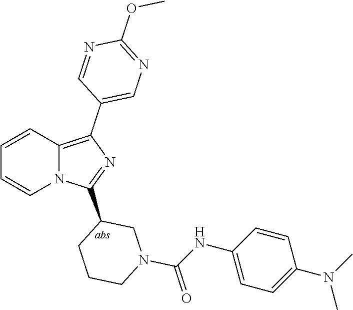 [SMILES: COc1ncc(-c2nc([C@@H]3CCCN(C(=O)Nc4ccc(N(C)C)cc4)C3)n3ccccc23)cn1] |
| 10-61 | 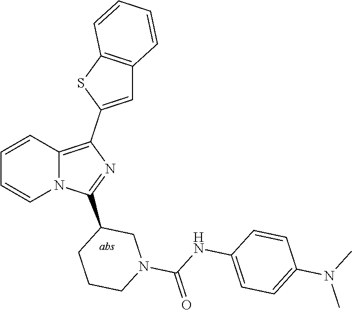 [SMILES: CN(C)c1ccc(NC(=O)N2CCC[C@@H](c3nc(-c4cc5ccccc5s4)c4ccccn34)C2)cc1] |
| 10-62 | 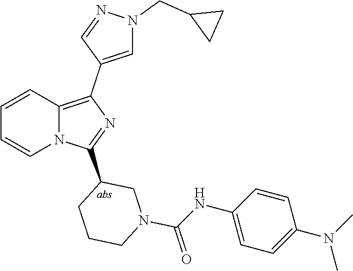 [SMILES: CN(C)c1ccc(NC(=O)N2CCC[C@@H](c3nc(-c4cnn(CC5CC5)c4)c4ccccn34)C2)cc1] |
| 10-63 | 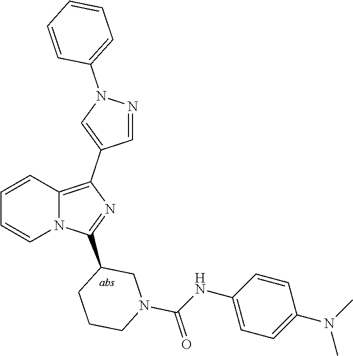 [SMILES: CN(C)c1ccc(NC(=O)N2CCC[C@@H](c3nc(-c4cnn(-c5ccccc5)c4)c4ccccn34)C2)cc1] |
| 10-64 | 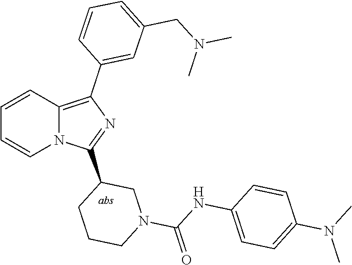 [SMILES: CN(C)Cc1cccc(-c2nc([C@@H]3CCCN(C(=O)Nc4ccc(N(C)C)cc4)C3)n3ccccc23)c1] |
| 10-65 | 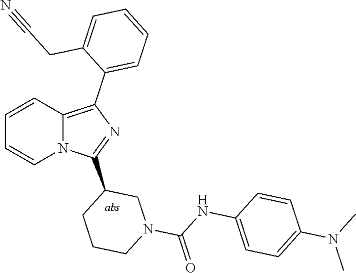 [SMILES: CN(C)c1ccc(NC(=O)N2CCC[C@@H](c3nc(-c4ccccc4CC#N)c4ccccn34)C2)cc1] |
| 10-66 | 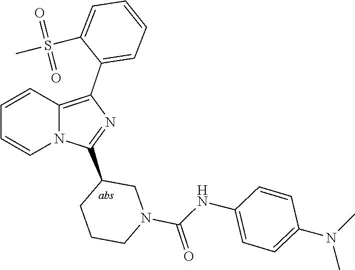 [SMILES: CN(C)c1ccc(NC(=O)N2CCC[C@@H](c3nc(-c4ccccc4S(C)(=O)=O)c4ccccn34)C2)cc1] |
| 10-67 | 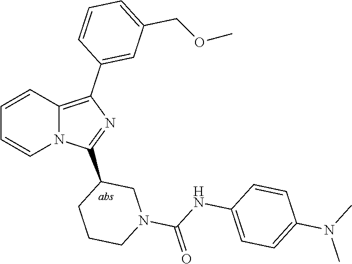 [SMILES: COCc1cccc(-c2nc([C@@H]3CCCN(C(=O)Nc4ccc(N(C)C)cc4)C3)n3ccccc23)c1] |
| 10-68 | 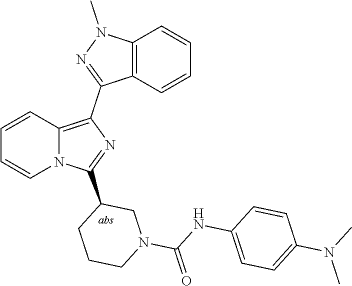 [SMILES: CN(C)c1ccc(NC(=O)N2CCC[C@@H](c3nc(-c4nn(C)c5ccccc45)c4ccccn34)C2)cc1] |
| 10-69 | 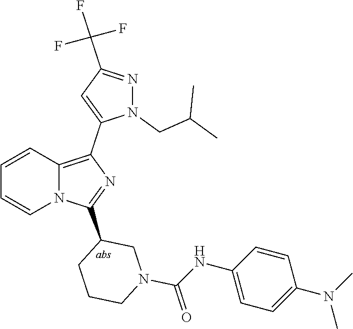 [SMILES: CC(C)Cn1nc(C(F)(F)F)cc1-c1nc([C@@H]2CCCN(C(=O)Nc3ccc(N(C)C)cc3)C2)n2ccccc12] |
| 10-70 | 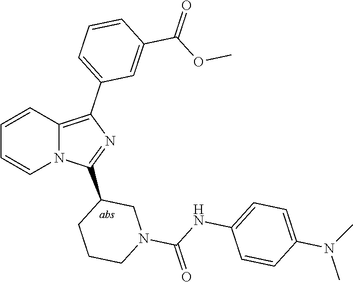 [SMILES: COC(=O)c1cccc(-c2nc([C@@H]3CCCN(C(=O)Nc4ccc(N(C)C)cc4)C3)n3ccccc23)c1] |
| 10-71 | 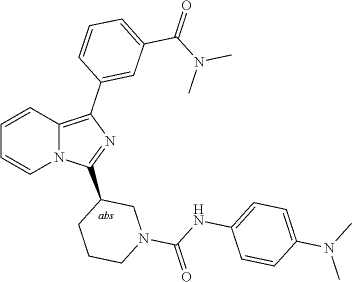 [SMILES: CN(C)C(=O)c1cccc(-c2nc([C@@H]3CCCN(C(=O)Nc4ccc(N(C)C)cc4)C3)n3ccccc23)c1] |
| 10-72 | 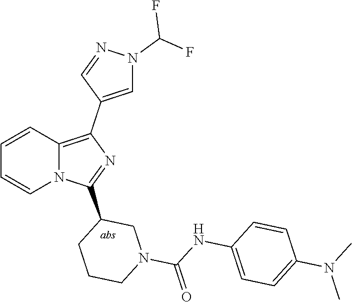 [SMILES: CN(C)c1ccc(NC(=O)N2CCC[C@@H](c3nc(-c4cnn(C(F)F)c4)c4ccccn34)C2)cc1] |
| 23-1 | 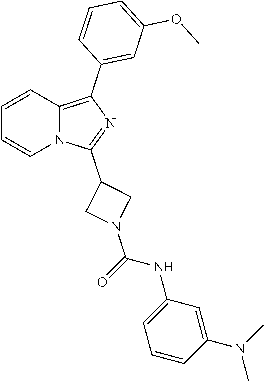 [SMILES: COc1cccc(-c2nc(C3CN(C(=O)Nc4cccc(N(C)C)c4)C3)n3ccccc23)c1] |
| 23-2 | 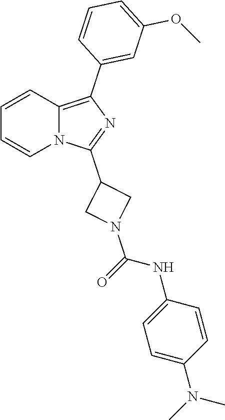 [SMILES: COc1cccc(-c2nc(C3CN(C(=O)Nc4ccc(N(C)C)cc4)C3)n3ccccc23)c1] |

In some embodiments, compounds having activity as modulators of the Mas-related G-protein coupled receptor D are provided, the compounds having the following structure (III):

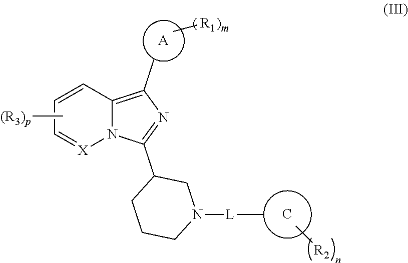

or a pharmaceutically acceptable salt, isomer, hydrate, solvate, or isotope thereof, wherein:
A is cycloalkyl or heterocyclyl;
C is carbocyclyl or heterocyclyl, or C is optionally absent when L is —C(O)—NRa- and is R₂ is alkyl, aminylalkyl, alkoxyalkyl, or alkylsufonylalkyl;
L is a linker having the structure —C(O)—, —C(O)-alkyl-, —C(O)—NRa-, —C(O)—NRa-alkyl-, —S(O)₂—, or —S(O)₂-alkyl-;
X is CR₃, CH, or N;
R₁ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, alkoxycarbonyl, aminylcarbonyl, alkylsulfonyl, cycloalkylalkyl, aryl, aralkyl, or arylsulfonyl;
R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
Ra is H or alkyl;
m is 0, 1, or 2;
n is 0, 1, 2, or 3; and
p is 0, 1, 2, or 3.

In some embodiments, compounds of structure (III) are provided wherein A is cycloalkyl. In other embodiments, compounds of structure (III) are provided wherein A is heterocyclyl. In other embodiments, compounds of structure (III) are provided wherein A is heteroaryl. In certain embodiments, compounds of structure (III) are provided wherein A is selected from:

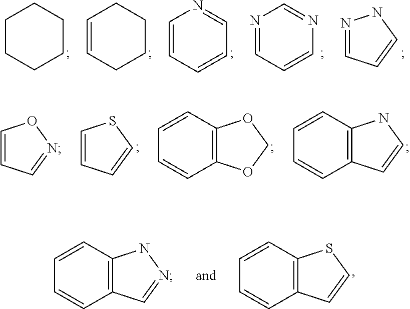

such that all valencies are satisfied.

In some embodiments, compounds of structure (III) are provided wherein C is carbocyclyl. In other embodiments, compounds of structure (III) are provided wherein C is cycloalkyl. In certain embodiments, compounds of structure (III) are provided wherein C is cyclopropyl, cyclobutyl, cyclopentyl, or cyclohexyl. In additional embodiments, compounds of structure (III) are provided wherein C is aryl. In specific embodiments, compounds of structure (III) are provided wherein C is phenyl. In yet other embodiments, compounds of structure (III) are provided wherein C is selected from:

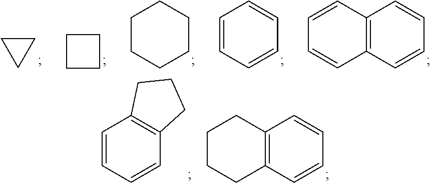

and

In further embodiments, compounds of structure (III) are provided wherein C is heterocyclyl. In some embodiments, compounds of structure (III) are provided wherein C is saturated heterocyclyl. In other embodiments, compounds of structure (III) are provided wherein C is heteroaryl. In certain embodiments, compounds of structure (III) are provided wherein C is selected from:

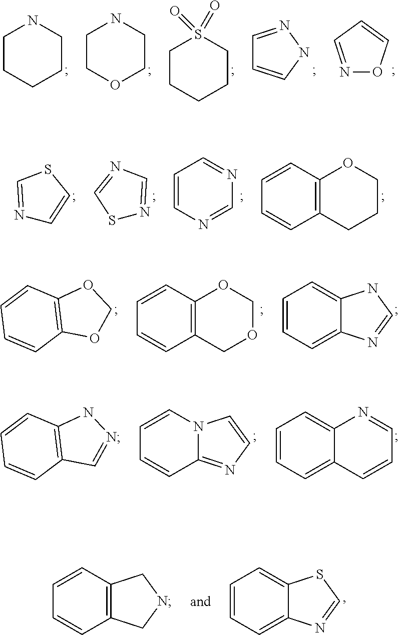

wherein all valencies are satisfied.

In some embodiments, compounds of structure (III) are provided wherein C is absent, L is —C(O)—NRa-, and R₂ is alkyl, aminylalkyl, alkoxyalkyl, or alkylsufonylalkyl. In other embodiments, compounds of structure (III) are provided wherein C is absent, L is —C(O)—NRa-, and R₂ is alkyl. In additional embodiments, compounds of structure (III) are provided wherein C is absent, L is —C(O)—NRa-, and R₂ is aminylalkyl. In yet other embodiments, compounds of structure (III) are provided wherein C is absent, L is —C(O)—NRa-, and R₂ is alkoxyalkyl. In further embodiments, compounds of structure (III) are provided wherein C is absent, L is —C(O)—NRa-, and R₂ is alkylsufonylalkyl.

In some embodiments, compounds of structure (III) are provided wherein L is a linker having the structure —C(O)—. In other embodiments, compounds of structure (III) are provided wherein L is a linker having the structure —C(O)-alkyl-. In additional embodiments, compounds of structure (III) are provided wherein L is a linker having the structure —C(O)—NRa-. In further embodiments, compounds of structure (III) are provided wherein L is a linker having the structure —C(O)—NRa-alkyl-. In yet other embodiments, compounds of structure (III) are provided wherein L is a linker having the structure —S(O)₂—. In some embodiments, compounds of structure (III) are provided wherein L is a linker having the structure —S(O)₂-alkyl-.

In some embodiments, compounds of structure (III) are provided wherein X is CR₃. In other embodiments, compounds of structure (III) are provided wherein X is CH. In additional embodiments, compounds of structure (III) are provided wherein X is N.

In some embodiments, compounds of structure (III) are provided wherein R₁ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, alkoxycarbonyl, aminylcarbonyl, or alkylsulfonyl. In other embodiments, compounds of structure (III) are provided wherein R₁ is cycloalkylalkyl, aryl, aralkyl, or arylsulfonyl. In certain embodiments, compounds of structure (III) are provided wherein R₁ is selected from:

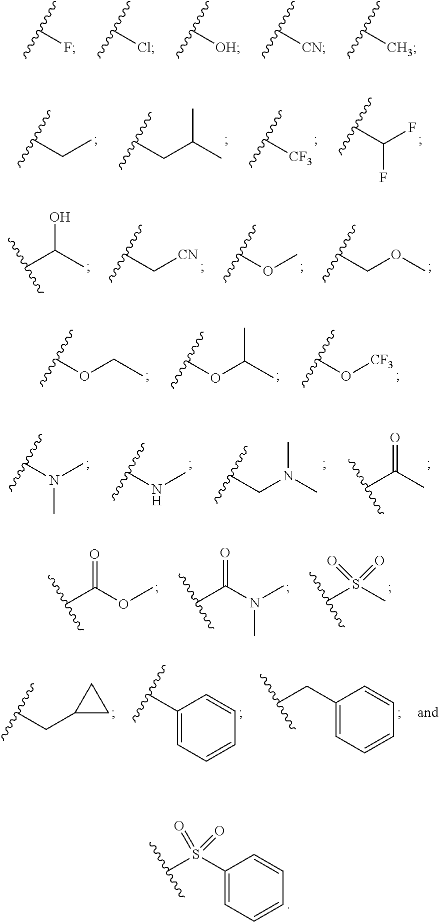

In specific embodiments, compounds of structure (III) are provided wherein R₁ is selected from:

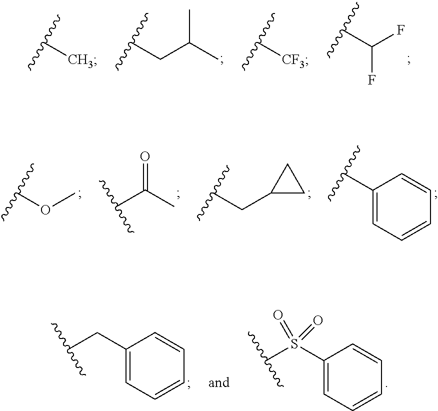

In some embodiments, compounds of structure (III) are provided wherein R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, or alkylsufonylalkyl. In some embodiments, compounds of structure (III) are provided wherein R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, or alkylsulfonyl. In other embodiments, compounds of structure (III) are provided wherein R₂ is aryl, heterocyclyl, or alkylheteroaryl. In certain embodiments, compounds of structure (III) are provided wherein R₂ is selected from:

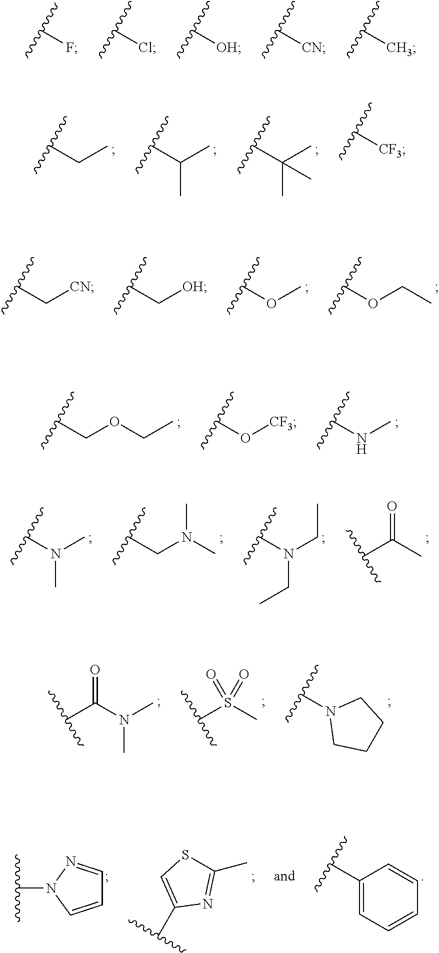

In specific embodiments, compounds of structure (III) are provided wherein R₂ is selected from: halo and aminyl. In specific embodiments, compounds of structure (III) are provided wherein R₂ is selected from:

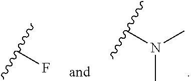

In some embodiments, compounds of structure (III) are provided wherein R₃ is alkyl. In other embodiments, compounds of structure (III) are provided wherein R₃ is alkoxy.

In some embodiments, compounds of structure (III) are provided wherein Ra is H. In other embodiments, compounds of structure (III) are provided wherein Ra is alkyl.

In some embodiments, compounds of structure (III) are provided wherein m is 0. In other embodiments, compounds of structure (III) are provided wherein m is 1. In additional embodiments, compounds of structure (III) are provided wherein m is 2.

In some embodiments, compounds of structure (III) are provided wherein n is 0. In other embodiments, compounds of structure (III) are provided wherein n is 1. In additional embodiments, compounds of structure (III) are provided wherein n is 2. In further embodiments, compounds of structure (III) are provided wherein n is 3.

In some embodiments, compounds of structure (III) are provided wherein p is 0. In other embodiments, compounds of structure (III) are provided wherein p is 1. In additional embodiments, compounds of structure (III) are provided wherein p is 2. In further embodiments, compounds of structure (III) are provided wherein p is 3.

Representative compounds of structure (III) include any one of the compounds listed in Table III below, as well as a pharmaceutically acceptable isomer, racemate, hydrate, solvate, isotope, or salt thereof.

TABLE III
Compounds of Structure III

| Cpd. No. | Structure | Cpd. No. | Structure |
|---|---|---|---|
| 10-27 | 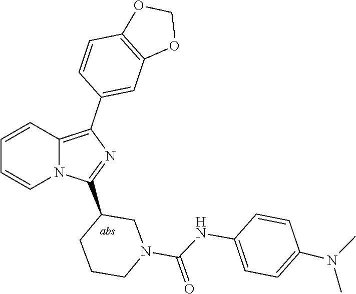 [SMILES: CN(C)c1ccc(NC(=O)N2CCC[C@@H](c3nc(-c4ccc5c(c4)OCO5)c4ccccn34)C2)cc1] | 10-53 | 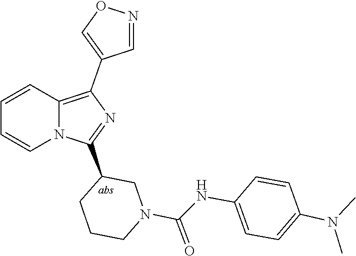 [SMILES: CN(C)c1ccc(NC(=O)N2CCC[C@@H](c3nc(-c4cnoc4)c4ccccn34)C2)cc1] |
| 10-44 | 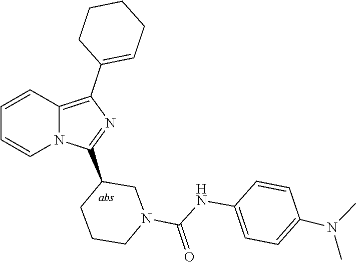 [SMILES: CN(C)c1ccc(NC(=O)N2CCC[C@@H](c3nc(C4=CCCCC4)c4ccccn34)C2)cc1] | 10-54 | 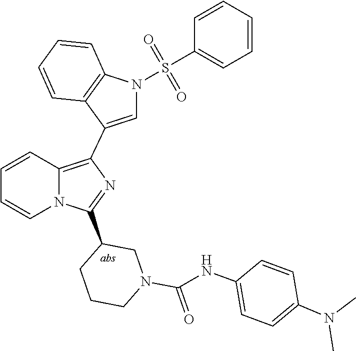 [SMILES: CN(C)c1ccc(NC(=O)N2CCC[C@@H](c3nc(-c4cn(S(=O)(=O)c5ccccc5)c5ccccc45)c4ccccn34)C2)cc1] |
| 10-45 | 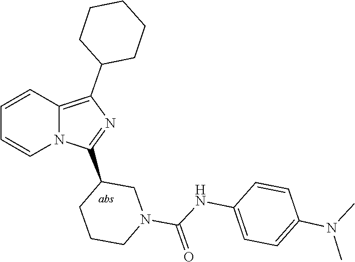 [SMILES: CN(C)c1ccc(NC(=O)N2CCC[C@@H](c3nc(C4CCCCC4)c4ccccn34)C2)cc1] | 10-55 | 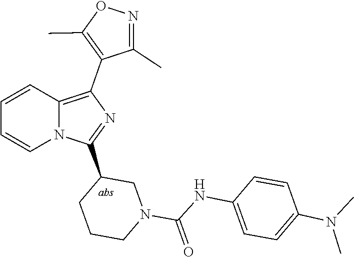 [SMILES: Cc1noc(C)c1-c1nc([C@@H]2CCCN(C(=O)Nc3ccc(N(C)C)cc3)C2)n2ccccc12] |
| 10-47 | 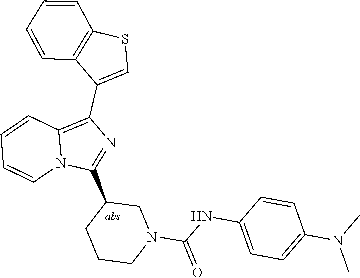 [SMILES: CN(C)c1ccc(NC(=O)N2CCC[C@@H](c3nc(-c4csc5ccccc45)c4ccccn34)C2)cc1] | 10-56 | 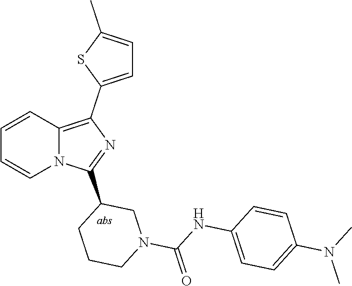 [SMILES: Cc1ccc(-c2nc([C@@H]3CCCN(C(=O)Nc4ccc(N(C)C)cc4)C3)n3ccccc23)s1] |
| 10-51 | 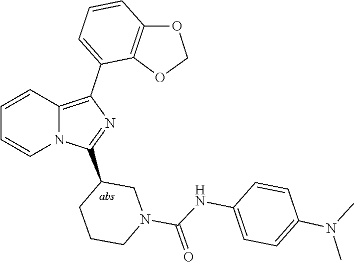 [SMILES: CN(C)c1ccc(NC(=O)N2CCC[C@@H](c3nc(-c4cccc5c4OCO5)c4ccccn34)C2)cc1] | 10-57 | 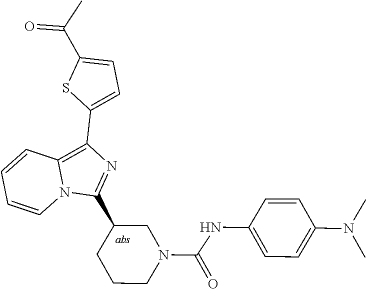 [SMILES: CC(=O)c1ccc(-c2nc([C@@H]3CCCN(C(=O)Nc4ccc(N(C)C)cc4)C3)n3ccccc23)s1] |
| 10-52 | 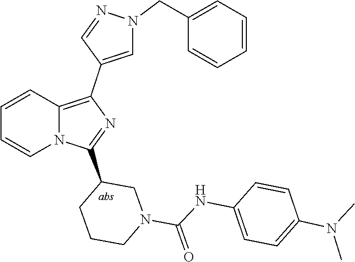 [SMILES: CN(C)c1ccc(NC(=O)N2CCC[C@@H](c3nc(-c4cnn(Cc5ccccc5)c4)c4ccccn34)C2)cc1] | 10-58 | 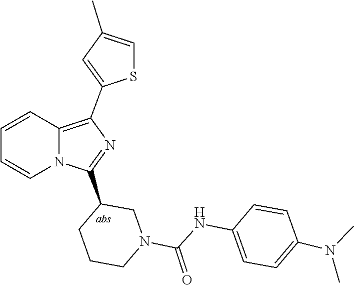 [SMILES: Cc1csc(-c2nc([C@@H]3CCCN(C(=O)Nc4ccc(N(C)C)cc4)C3)n3ccccc23)c1] |
| 10-60 | 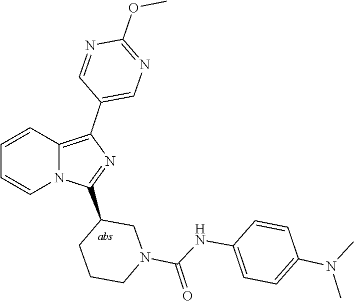 [SMILES: COc1ncc(-c2nc([C@@H]3CCCN(C(=O)Nc4ccc(N(C)C)cc4)C3)n3ccccc23)cn1] | 10-72 | 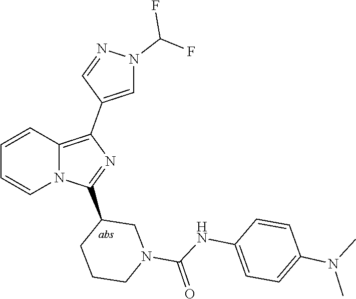 [SMILES: CN(C)c1ccc(NC(=O)N2CCC[C@@H](c3nc(-c4cnn(C(F)F)c4)c4ccccn34)C2)cc1] |
| 10-61 | 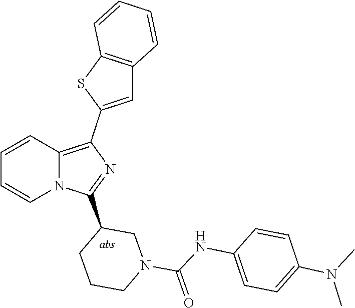 [SMILES: CN(C)c1ccc(NC(=O)N2CCC[C@@H](c3nc(-c4cc5ccccc5s4)c4ccccn34)C2)cc1] | 11-6 | 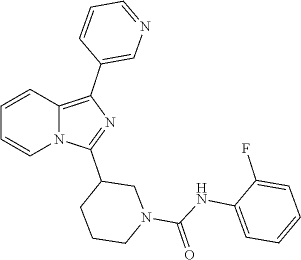 [SMILES: O=C(Nc1ccccc1F)N1CCCC(c2nc(-c3cccnc3)c3ccccn23)C1] |
| 10-62 | 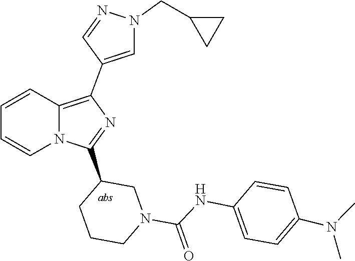 [SMILES: CN(C)c1ccc(NC(=O)N2CCC[C@@H](c3nc(-c4cnn(CC5CC5)c4)c4ccccn34)C2)cc1] | 11-10 | 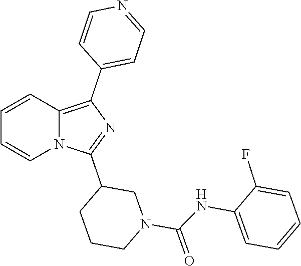 [SMILES: O=C(Nc1ccccc1F)N1CCCC(c2nc(-c3ccncc3)c3ccccn23)C1] |
| 10-63 | 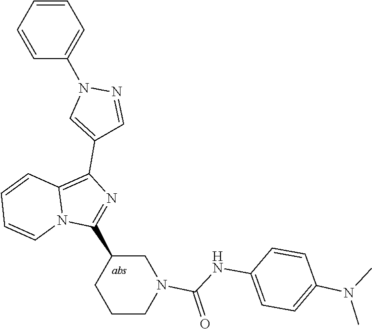 [SMILES: CN(C)c1ccc(NC(=O)N2CCC[C@@H](c3nc(-c4cnn(-c5ccccc5)c4)c4ccccn34)C2)cc1] | 11-11 | 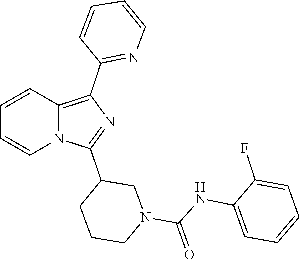 [SMILES: O=C(Nc1ccccc1F)N1CCCC(c2nc(-c3ccccn3)c3ccccn23)C1] |
| 10-68 | 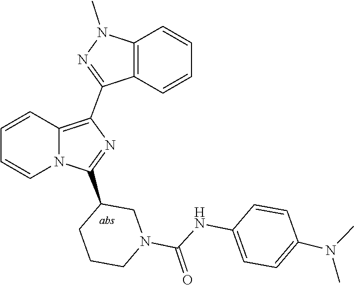 [SMILES: CN(C)c1ccc(NC(=O)N2CCC[C@@H](c3nc(-c4nn(C)c5ccccc45)c4ccccn34)C2)cc1] | | |
| 10-69 | 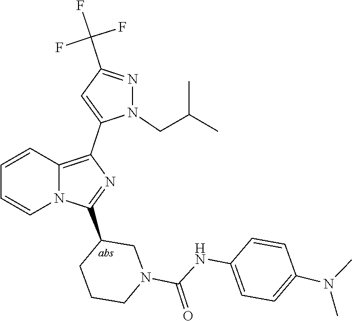 [SMILES: CC(C)Cn1nc(C(F)(F)F)cc1-c1nc([C@@H]2CCCN(C(=O)Nc3ccc(N(C)C)cc3)C2)n2ccccc12] | | |

In some embodiments, compounds having activity as modulators of the Mas-related G-protein coupled receptor D are provided, the compounds having the following structure (IV):

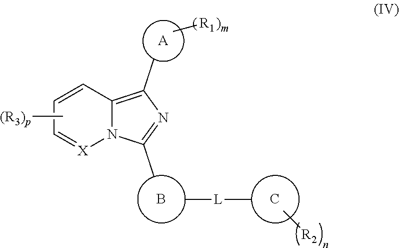

or a pharmaceutically acceptable salt, isomer, hydrate, solvate, or isotope thereof, wherein:
A is carbocyclyl or heterocyclyl;
B is heterocyclyl;
C is a nitrogen containing heterocyclyl;
L is a linker having the structure —C(O)—, —C(O)-alkyl-, —C(O)—NRa-, —C(O)—NRa-alkyl-, —S(O)₂—, or —S(O)₂-alkyl-;
X is CR₃, CH, or N;
R₁ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, alkoxycarbonyl, aminylcarbonyl, alkylsulfonyl, cycloalkylalkyl, aryl, aralkyl, or arylsulfonyl;
R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
Ra is H or alkyl;
m is 0, 1, or 2;
n is 0, 1, 2, or 3; and
p is 0, 1, 2, or 3.

In some embodiments, compounds of structure (IV) are provided wherein A is carbocyclyl. In some embodiments, compounds of structure (IV) are provided wherein A is cycloalkyl. In other embodiments, compounds of structure (IV) are provided wherein A is aryl. In certain embodiments, compounds of structure (IV) are provided wherein A is phenyl. In additional embodiments, compounds of structure (IV) are provided wherein A is heterocyclyl. In additional embodiments, compounds of structure (IV) are provided wherein A is heteroaryl. In certain embodiments, compounds of structure (IV) are provided wherein A is selected from:

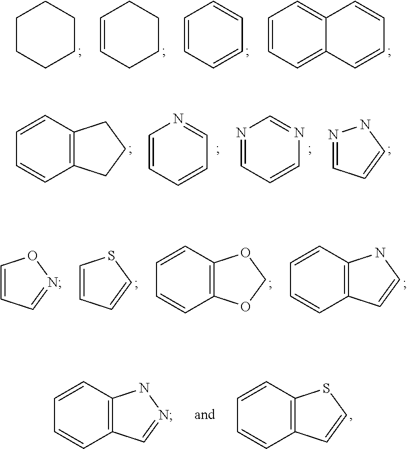

such that all valencies are satisfied.

In some embodiments, compounds of structure (IV) are provided wherein B is a saturated heterocyclyl. In other embodiments, compounds of structure (IV) are provided wherein B is a partially unsaturated heterocyclyl. In additional embodiments, compounds of structure (IV) are provided wherein B is heteroaryl. In certain embodiments, compounds of structure (IV) are provided wherein B is thiophenyl. In further embodiments, compounds of structure (IV) are provided wherein B is piperidinyl. In yet other embodiments, compounds of structure (IV) are provided wherein B is pyrrolidinyl. In some embodiments, compounds of structure (IV) are provided wherein B is azetidinyl.

In some embodiments, compounds of structure (IV) are provided wherein C is a saturated nitrogen containing heterocyclyl. In other embodiments, compounds of structure (IV) are provided wherein C is a partially unsaturated nitrogen containing heterocyclyl. In additional embodiments, compounds of structure (IV) are provided wherein C is a nitrogen containing heteroaryl. In certain embodiments, compounds of structure (IV) are provided wherein C is selected from:

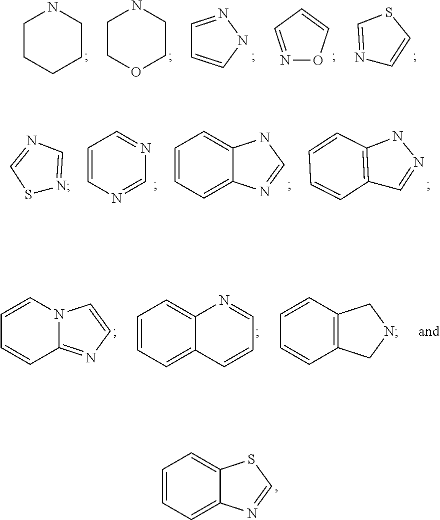

wherein all valencies are satisfied.

In some embodiments, compounds of structure (IV) are provided wherein L is a linker having the structure —C(O)—. In other embodiments, compounds of structure (IV) are provided wherein L is a linker having the structure —C(O)-alkyl-. In additional embodiments, compounds of structure (IV) are provided wherein L is a linker having the structure —C(O)—NRa-. In further embodiments, compounds of structure (IV) are provided wherein L is a linker having the structure —C(O)—NRa-alkyl-. In yet other embodiments, compounds of structure (IV) are provided wherein L is a linker having the structure —S(O)₂—. In some embodiments, compounds of structure (IV) are provided wherein L is a linker having the structure —S(O)₂-alkyl-.

In some embodiments, compounds of structure (IV) are provided wherein X is CR₃. In other embodiments, compounds of structure (IV) are provided wherein X is CH. In additional embodiments, compounds of structure (IV) are provided wherein X is N.

In some embodiments, compounds of structure (IV) are provided wherein R₁ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, alkoxycarbonyl, aminylcarbonyl, or alkylsulfonyl. In other embodiments, compounds of structure (IV) are provided wherein R₁ is cycloalkylalkyl, aryl, aralkyl, or arylsulfonyl. In additional embodiments, compounds of structure (IV) are provided wherein R₁ is selected from:

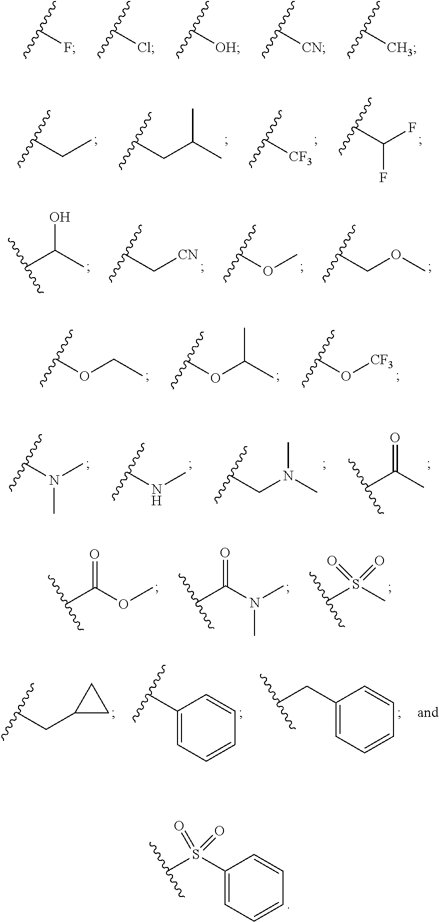

In further embodiments, compounds of structure (IV) are provided wherein R₁ is

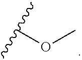 [SMILES: *OC]

In some embodiments, compounds of structure (IV) are provided wherein R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, or alkylsufonylalkyl. In some embodiments, compounds of structure (IV) are provided wherein R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, or alkylsulfonyl. In other embodiments, compounds of structure (IV) are provided wherein R₂ is aryl, heterocyclyl, or alkylheteroaryl. In certain embodiments, compounds of structure (IV) are provided wherein R₂ is selected from:

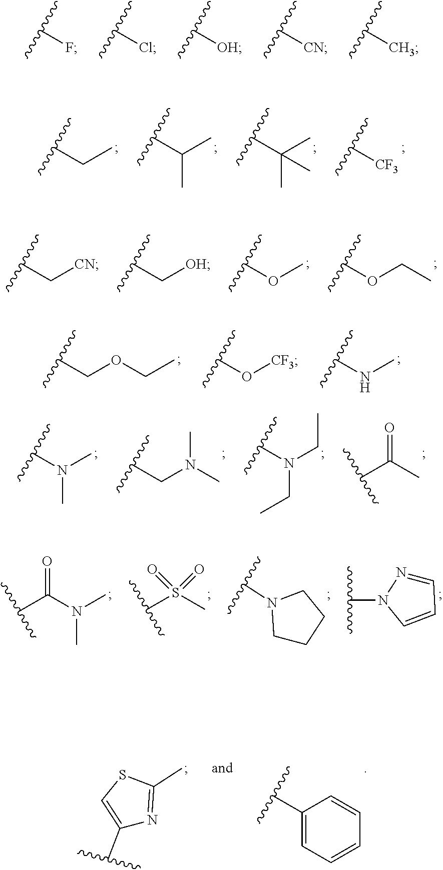

In specific embodiments, compounds of structure (IV) are provided wherein R₂ is selected from: cyano, alkyl, alkoxyalkyl, and aminyl. In specific embodiments, compounds of structure (IV) are provided wherein R₂ is selected from:

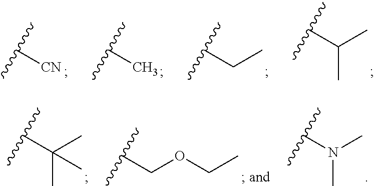

In some embodiments, compounds of structure (IV) are provided wherein R₃ is alkyl. In other embodiments, compounds of structure (IV) are provided wherein R₃ is alkoxy.

In some embodiments, compounds of structure (IV) are provided wherein Ra is H. In other embodiments, compounds of structure (IV) are provided wherein Ra is alkyl.

In some embodiments, compounds of structure (IV) are provided wherein m is 0. In other embodiments, compounds of structure (IV) are provided wherein m is 1. In additional embodiments, compounds of structure (IV) are provided wherein m is 2.

In some embodiments, compounds of structure (IV) are provided wherein n is 0. In other embodiments, compounds of structure (IV) are provided wherein n is 1. In additional embodiments, compounds of structure (IV) are provided wherein n is 2. In further embodiments, compounds of structure (IV) are provided wherein n is 3.

In some embodiments, compounds of structure (IV) are provided wherein p is 0. In other embodiments, compounds of structure (IV) are provided wherein p is 1. In additional embodiments, compounds of structure (IV) are provided wherein p is 2. In further embodiments, compounds of structure (IV) are provided wherein p is 3.

Representative compounds of structure (IV) include any one of the compounds listed in Table IV below, as well as a pharmaceutically acceptable isomer, racemate, hydrate, solvate, isotope, or salt thereof.

TABLE IV
Compounds of Structure IV

| Cpd. No. | Structure |
|---|---|
| 1-28 | 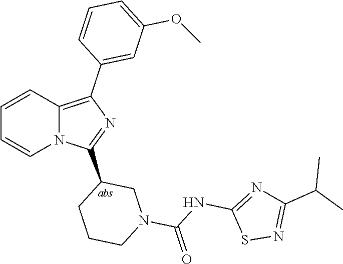 [SMILES: COc1cccc(-c2nc([C@@H]3CCCN(C(=O)Nc4nc(C(C)C)ns4)C3)n3ccccc23)c1] |
| 1-30 | 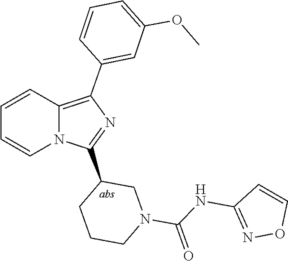 [SMILES: COc1cccc(-c2nc([C@@H]3CCCN(C(=O)Nc4ccon4)C3)n3ccccc23)c1] |
| 1-32 | 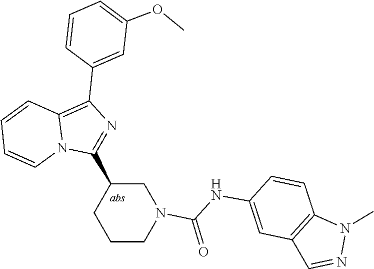 [SMILES: COc1cccc(-c2nc([C@@H]3CCCN(C(=O)Nc4ccc5c(cnn5C)c4)C3)n3ccccc23)c1] |
| 1-33 | 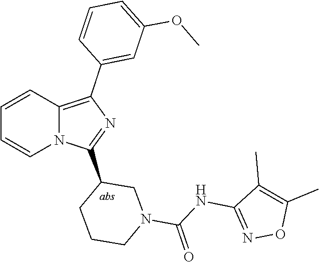 [SMILES: COc1cccc(-c2nc([C@@H]3CCCN(C(=O)Nc4noc(C)c4C)C3)n3ccccc23)c1] |
| 5-30 | 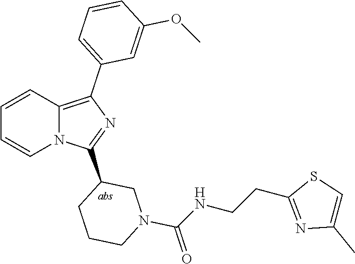 [SMILES: COc1cccc(-c2nc([C@@H]3CCCN(C(=O)NCCc4nc(C)cs4)C3)n3ccccc23)c1] |
| 5-40 | 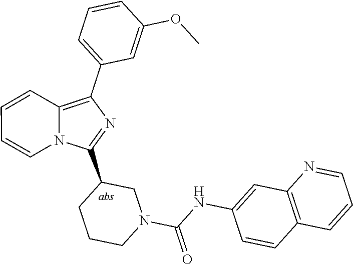 [SMILES: COc1cccc(-c2nc([C@@H]3CCCN(C(=O)Nc4ccc5cccnc5c4)C3)n3ccccc23)c1] |
| 5-42 | 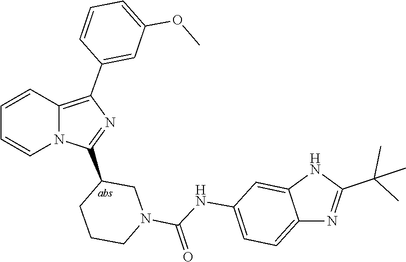 [SMILES: COc1cccc(-c2nc([C@@H]3CCCN(C(=O)Nc4ccc5nc(C(C)(C)C)[nH]c5c4)C3)n3ccccc23)c1] |
| 5-44 | 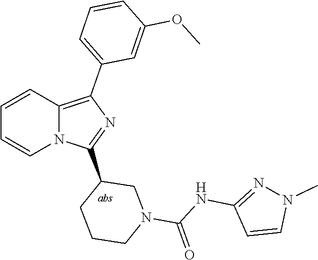 [SMILES: COc1cccc(-c2nc([C@@H]3CCCN(C(=O)Nc4ccn(C)n4)C3)n3ccccc23)c1] |
| 5-45 | 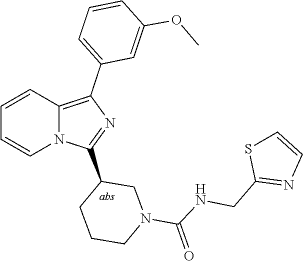 [SMILES: COc1cccc(-c2nc([C@@H]3CCCN(C(=O)NCc4nccs4)C3)n3ccccc23)c1] |
| 5-46 | 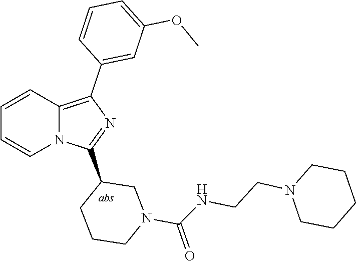 [SMILES: COc1cccc(-c2nc([C@@H]3CCCN(C(=O)NCCN4CCCCC4)C3)n3ccccc23)c1] |
| 5-49 | 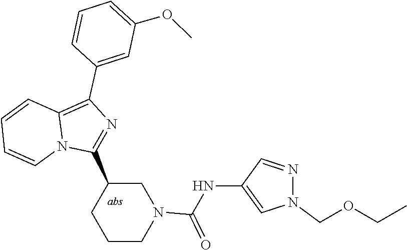 [SMILES: CCOCn1cc(NC(=O)N2CCC[C@@H](c3nc(-c4cccc(OC)c4)c4ccccn34)C2)cn1] |
| 5-50 | 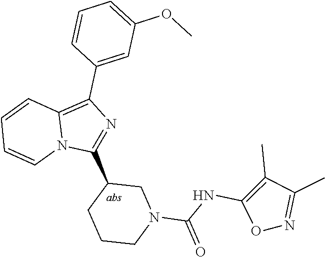 [SMILES: COc1cccc(-c2nc([C@@H]3CCCN(C(=O)Nc4onc(C)c4C)C3)n3ccccc23)c1] |
| 5-51 | 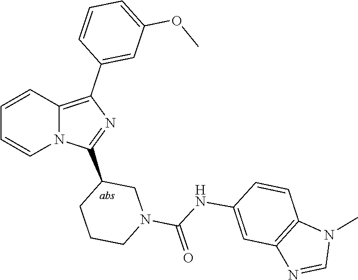 [SMILES: COc1cccc(-c2nc([C@@H]3CCCN(C(=O)Nc4ccc5c(c4)ncn5C)C3)n3ccccc23)c1] |
| 5-53 | 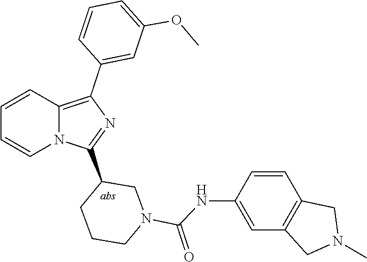 [SMILES: COc1cccc(-c2nc([C@@H]3CCCN(C(=O)Nc4ccc5c(c4)CN(C)C5)C3)n3ccccc23)c1] |
| 5-54 | 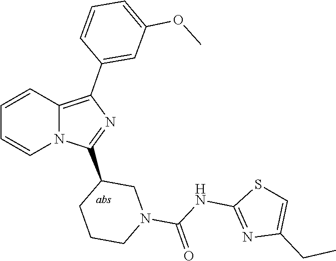 [SMILES: CCc1csc(NC(=O)N2CCC[C@@H](c3nc(-c4cccc(OC)c4)c4ccccn34)C2)n1] |
| 5-59 | 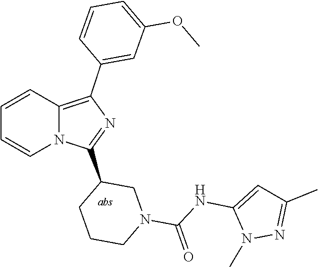 [SMILES: COc1cccc(-c2nc([C@@H]3CCCN(C(=O)Nc4cc(C)nn4C)C3)n3ccccc23)c1] |
| 5-62 | 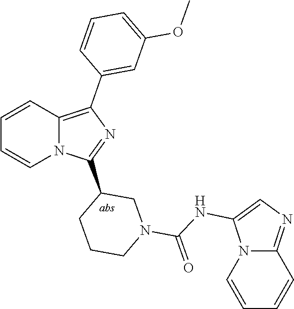 [SMILES: COc1cccc(-c2nc([C@@H]3CCCN(C(=O)Nc4cnc5ccccn45)C3)n3ccccc23)c1] |
| 5-64 | 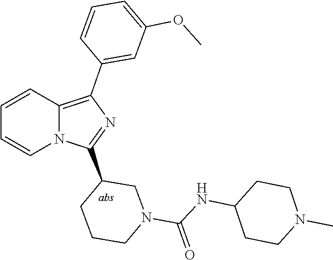 [SMILES: COc1cccc(-c2nc([C@@H]3CCCN(C(=O)NC4CCN(C)CC4)C3)n3ccccc23)c1] |
| 5-65 | 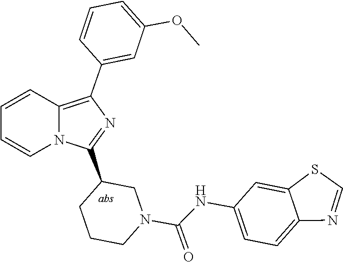 [SMILES: COc1cccc(-c2nc([C@@H]3CCCN(C(=O)Nc4ccc5ncsc5c4)C3)n3ccccc23)c1] |
| 5-66 | 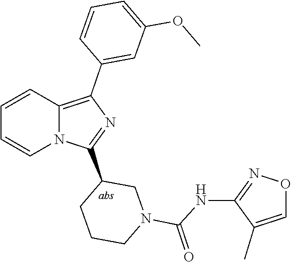 [SMILES: COc1cccc(-c2nc([C@@H]3CCCN(C(=O)Nc4nocc4C)C3)n3ccccc23)c1] |
| 5-67 | 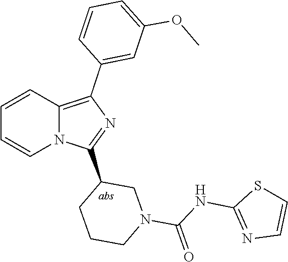 [SMILES: COc1cccc(-c2nc([C@@H]3CCCN(C(=O)Nc4nccs4)C3)n3ccccc23)c1] |
| 5-68 | 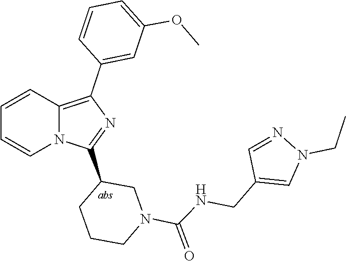 [SMILES: CCn1cc(CNC(=O)N2CCC[C@@H](c3nc(-c4cccc(OC)c4)c4ccccn34)C2)cn1] |
| 5-69 | 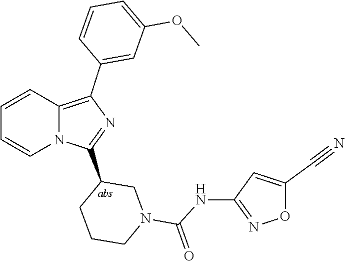 [SMILES: COc1cccc(-c2nc([C@@H]3CCCN(C(=O)Nc4cc(C#N)on4)C3)n3ccccc23)c1] |
| 5-72 | 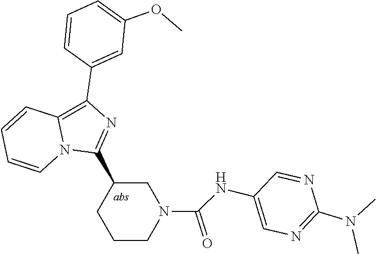 [SMILES: COc1cccc(-c2nc([C@@H]3CCCN(C(=O)Nc4cnc(N(C)C)nc4)C3)n3ccccc23)c1] |
| 5-73 | 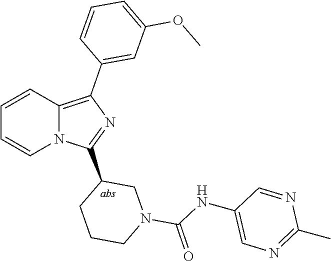 [SMILES: COc1cccc(-c2nc([C@@H]3CCCN(C(=O)Nc4cnc(C)nc4)C3)n3ccccc23)c1] |
| 10-25 | 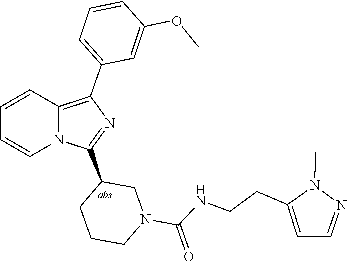 [SMILES: COc1cccc(-c2nc([C@@H]3CCCN(C(=O)NCCc4ccnn4C)C3)n3ccccc23)c1] |
| 10-38 | 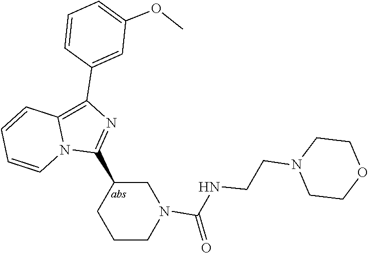 [SMILES: COc1cccc(-c2nc([C@@H]3CCCN(C(=O)NCCN4CCOCC4)C3)n3ccccc23)c1] |

In some embodiments, compounds having activity as modulators of the Mas-related G-protein coupled receptor D are provided, the compounds having the following structure (V):

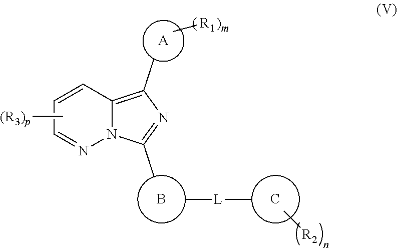

or a pharmaceutically acceptable salt, isomer, hydrate, solvate, or isotope thereof, wherein:
A and B are each individually carbocyclyl or heterocyclyl;
C is carbocyclyl or heterocyclyl, or C is optionally absent when L is —C(O)—NRa- and R₂ is alkyl, aminylalkyl, alkoxyalkyl, or alkylsufonylalkyl;
L is a linker having the structure —C(O)—, —C(O)-alkyl-, —C(O)—NRa-, —C(O)—NRa-alkyl-, —S(O)₂—, or —S(O)₂-alkyl-;
R₁ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, alkoxycarbonyl, aminylcarbonyl, alkylsulfonyl, cycloalkylalkyl, aryl, aralkyl, or arylsulfonyl;
R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
Ra is H or alkyl;
m is 0, 1, or 2;
n is 0, 1, 2, or 3; and
p is 0, 1, 2, or 3.

In some embodiments, compounds of structure (V) are provided wherein A is carbocyclyl. In some embodiments, compounds of structure (V) are provided wherein A is cycloalkyl. In other embodiments, compounds of structure (V) are provided wherein A is aryl. In certain embodiments, compounds of structure (V) are provided wherein A is phenyl. In additional embodiments, compounds of structure (V) are provided wherein A is heterocyclyl. In additional embodiments, compounds of structure (V) are provided wherein A is heteroaryl. In further embodiments, compounds of structure (V) are provided wherein A is selected from:

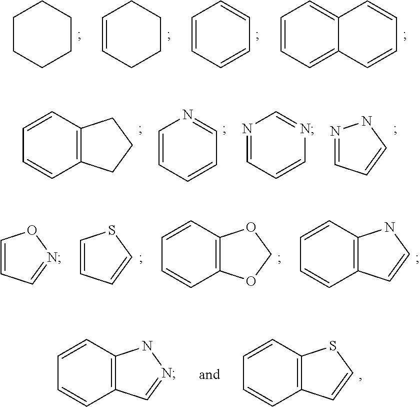

such that all valencies are satisfied.

In some embodiments, compounds of structure (V) are provided wherein B is carbocyclyl. In some embodiments, compounds of structure (V) are provided wherein B is cycloalkyl. In certain embodiments, compounds of structure (V) are provided wherein B is aryl. In specific embodiments, compounds of structure (V) are provided wherein B is phenyl. In additional embodiments, compounds of structure (V) are provided wherein B is heterocyclyl. In additional embodiments, compounds of structure (V) are provided wherein B is heteroaryl. In further embodiments, compounds of structure (V) are provided wherein B is thiophenyl. In yet other embodiments, compounds of structure (V) are provided wherein B is piperidinyl. In some embodiments, compounds of structure (V) are provided wherein B is pyrrolidinyl. In other embodiments, compounds of structure (V) are provided wherein B is azetidinyl.

In some embodiments, compounds of structure (V) are provided wherein C is carbocyclyl. In other embodiments, compounds of structure (V) are provided wherein C is cycloalkyl. In certain embodiments, compounds of structure (V) are provided wherein C is cyclopropyl, cyclobutyl, cyclopentyl, or cyclohexyl. In additional embodiments, compounds of structure (V) are provided wherein C is aryl. In specific embodiments, compounds of structure (V) are provided wherein C is phenyl. In certain embodiments, compounds of structure (V) are provided wherein C is selected from:

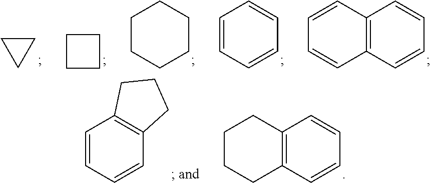

In further embodiments, compounds of structure (V) are provided wherein C is heterocyclyl. In some embodiments, compounds of structure (V) are provided wherein C is saturated heterocyclyl. In other embodiments, compounds of structure (V) are provided wherein C is heteroaryl. In certain embodiments, compounds of structure (V) are provided wherein C is selected from:

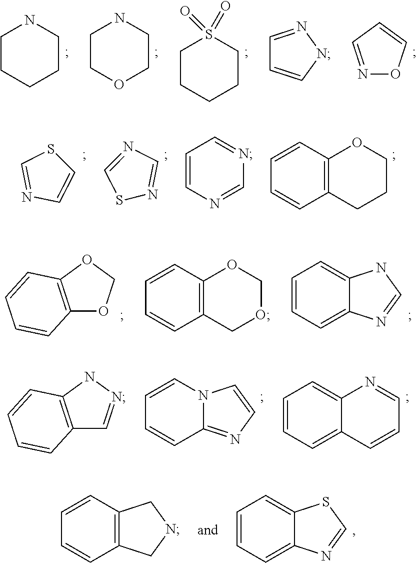

wherein all valencies are satisfied.

In some embodiments, compounds of structure (V) are provided wherein C is absent, L is —C(O)—NRa-, and R₂ is alkyl, aminylalkyl, alkoxyalkyl, or alkylsufonylalkyl. In other embodiments, compounds of structure (V) are provided wherein C is absent, L is —C(O)—NRa-, and R₂ is alkyl. In additional embodiments, compounds of structure (V) are provided wherein C is absent, L is —C(O)—NRa-, and R₂ is aminylalkyl. In additional embodiments, compounds of structure (V) are provided wherein C is absent, L is —C(O)—NRa-, and R₂ is alkoxyalkyl. In further embodiments, compounds of structure (V) are provided wherein C is absent, L is —C(O)—NRa-, and R₂ is alkylsufonylalkyl.

In some embodiments, compounds of structure (V) are provided wherein L is a linker having the structure —C(O)—. In other embodiments, compounds of structure (V) are provided wherein L is a linker having the structure —C(O)-alkyl-. In additional embodiments, compounds of structure (V) are provided wherein L is a linker having the structure —C(O)—NRa-. In further embodiments, compounds of structure (V) are provided wherein L is a linker having the structure —C(O)—NRa-alkyl-. In yet tother embodiments, compounds of structure (V) are provided wherein L is a linker having the structure —S(O)₂—. In some embodiments, compounds of structure (V) are provided wherein L is a linker having the structure —S(O)₂-alkyl-.

In some embodiments, compounds of structure (V) are provided wherein R₁ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, alkoxycarbonyl, aminylcarbonyl, or alkylsulfonyl. In other embodiments, compounds of structure (V) are provided wherein R₁ is cycloalkylalkyl, aryl, aralkyl, or arylsulfonyl. In certain embodiments, compounds of structure (V) are provided wherein R₁ is selected from:

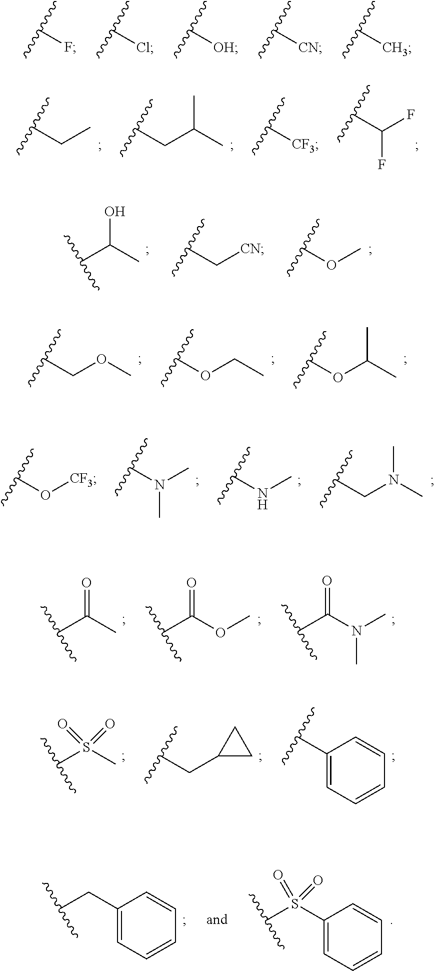

In specific embodiments, compounds of structure (V) are provided wherein R₁ is alkoxy. In specific embodiments, compounds of structure (V) are provided wherein R₁ is

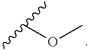 [SMILES: *OC]

In some embodiments, compounds of structure (V) are provided wherein R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, or alkylsufonylalkyl. In some embodiments, compounds of structure (V) are provided wherein R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, or alkylsulfonyl. In other embodiments, compounds of structure (V) are provided wherein R₂ is aryl, heterocyclyl, or alkylheteroaryl. In certain embodiments, compounds of structure (V) are provided wherein R₂ is selected from:

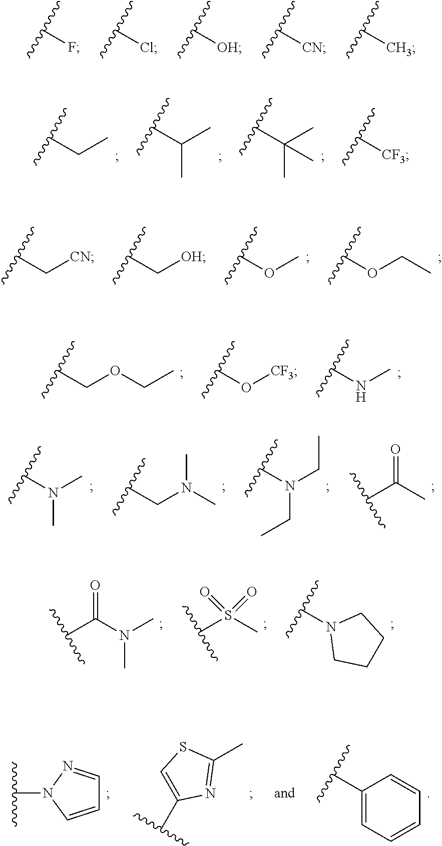

In specific embodiments, compounds of structure (V) are provided wherein R₂ is aminyl. In specific embodiments, compounds of structure (V) are provided wherein R₂ is

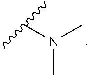 [SMILES: *N(C)C]

In some embodiments, compounds of structure (V) are provided wherein R₃ is alkyl. In other embodiments, compounds of structure (V) are provided wherein R₃ is alkoxy.

In some embodiments, compounds of structure (V) are provided wherein Ra is H. In other embodiments, compounds of structure (V) are provided wherein Ra is alkyl.

In some embodiments, compounds of structure (V) are provided wherein m is 0. In other embodiments, compounds of structure (V) are provided wherein m is 1. In additional embodiments, compounds of structure (V) are provided wherein m is 2.

In some embodiments, compounds of structure (V) are provided wherein n is 0. In other embodiments, compounds of structure (V) are provided wherein n is 1. In additional embodiments, compounds of structure (V) are provided wherein n is 2. In further embodiments, compounds of structure (V) are provided wherein n is 3.

In some embodiments, compounds of structure (V) are provided wherein p is 0. In other embodiments, compounds of structure (V) are provided wherein p is 1. In additional embodiments, compounds of structure (V) are provided wherein p is 2. In further embodiments, compounds of structure (V) are provided wherein p is 3.

Representative compounds of structure (V) include any one of the compounds listed in Table V below, as well as a pharmaceutically acceptable isomer, racemate, hydrate, solvate, isotope, or salt thereof.

TABLE V
Compounds of Structure V

| Cpd. No. | Structure |
|---|---|
| 5-19 | 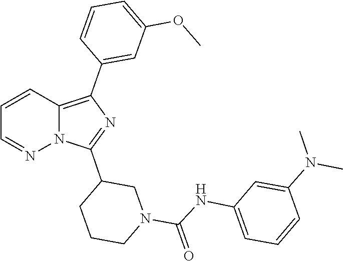 [SMILES: COc1cccc(-c2nc(C3CCCN(C(=O)Nc4cccc(N(C)C)c4)C3)n3ncccc23)c1] |
| 5-20 | 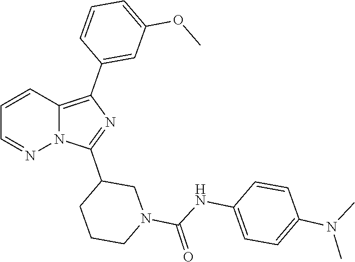 [SMILES: COc1cccc(-c2nc(C3CCCN(C(=O)Nc4ccc(N(C)C)cc4)C3)n3ncccc23)c1] |

In some embodiments, compounds having activity as modulators of the Mas-related G-protein coupled receptor D are provided, the compounds having the following structure (VI):

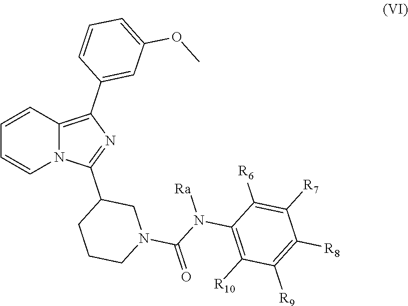

or a pharmaceutically acceptable salt, isomer, hydrate, solvate, or isotope thereof, wherein:

Ra is H or alkyl; and
either (1) R₆-R₁₀ are each individually H;
(2) R₆ and R₁₀ are H and R₇, R₈, and R₉ are each individually methoxy;
(3) R₆ is methyl and R₇ is methoxy and R₈-R₁₀ are each individually H;
(4) R₉ and R₁₀ are both H, and all but one of R₆-R₈ is H and the non-hydrogen substituent is selected from:
R₆ is F, Cl, methyl, ethyl, methoxy, ethoxy, OCF₃, —C(O)CH₃,
R₇ is Cl, cyano, ethyl, CF₃, OCF₃, ethoxy, —C(O)CH₃, and
R₈ is F, Cl, methyl, ethyl, methoxy, ethoxy, OCF₃, phenyl;
(5) R₆ and R₇ join together with the phenyl ring to which they are attached to form

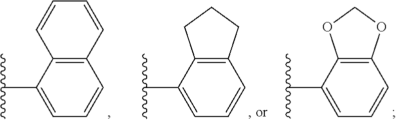

(6) R₇ and R₈ join together with the phenyl ring to which they are attached to form

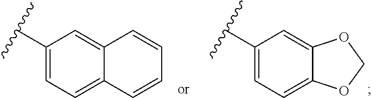 [SMILES: *c1ccc2ccccc2c1 ; *c1ccc2c(c1)OCO2]

or
(7) R₈ and R₉ join together with the phenyl ring to which they are attached to form

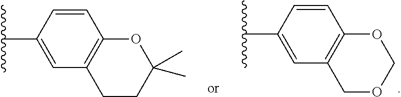 [SMILES: *c1ccc2c(c1)CCC(C)(C)O2 ; *c1ccc2c(c1)COCO2]

In some embodiments, compounds of structure (VI) are provided wherein Ra is H. In other embodiments, compounds of structure (VI) are provided wherein Ra is alkyl.

In some embodiments, compounds of structure (VI) are provided wherein R₆-R₁₀ are each individually H.

In some embodiments, compounds of structure (VI) are provided wherein R₆ and R₁₀ are H and R₇, R₈, and R₉ are each individually methoxy.

In some embodiments, compounds of structure (VI) are provided wherein R₆ is methyl and R₇ is methoxy and R₈-R₁₀ are each individually H.

In some embodiments, compounds of structure (VI) are provided wherein R₉ and R₁₀ are both H, and all but one of R₆-R₈ is H and the non-hydrogen substituent is selected from:
R₆ is F, Cl, methyl, ethyl, methoxy, ethoxy, OCF₃, —C(O)CH₃,
R₇ is Cl, cyano, ethyl, CF₃, OCF₃, ethoxy, —C(O)CH₃, and
R₈ is F, Cl, methyl, ethyl, methoxy, ethoxy, OCF₃, phenyl.

In certain embodiments, compounds of structure (VI) are provided wherein R₇, R₈, R₉, and R₁₀ are all H, and R₆ is F, Cl, methyl, ethyl, methoxy, ethoxy, OCF₃, or —C(O)CH₃. In specific embodiments, compounds of structure (VI) are provided wherein R₆, R₈, R₉, and R₁₀ are all H, and R₇ is Cl, cyano, ethyl, CF₃, OCF₃, ethoxy, or —C(O)CH₃. In yet other embodiments, compounds of structure (VI) are provided wherein R₆, R₇, R₉, and R₁₀ are all H, and R₈ is F, Cl, methyl, ethyl, methoxy, ethoxy, OCF₃, or phenyl.

In some embodiments, compounds of structure (VI) are provided wherein R₆ and R₇ join together with the phenyl ring to which they are attached to form

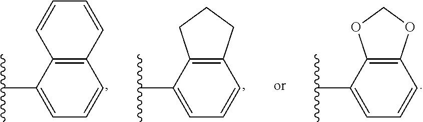

In some embodiments, compounds of structure (VI) are provided wherein R₇ and R₈ join together with the phenyl ring to which they are attached to form

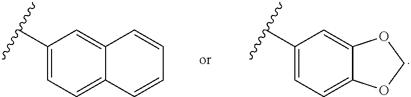 [SMILES: *c1ccc2ccccc2c1 ; *c1ccc2c(c1)OCO2]

In some embodiments, compounds of structure (VI) are provided wherein R₈ and R₉ join together with the phenyl ring to which they are attached to form

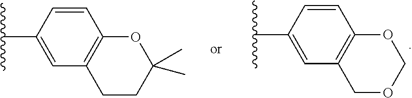 [SMILES: *c1ccc2c(c1)CCC(C)(C)O2 ; *c1ccc2c(c1)COCO2]

Representative compounds of structure (VI) include any one of the compounds listed in Table VI below, as well as a pharmaceutically acceptable isomer, racemate, hydrate, solvate, isotope, or salt thereof.

TABLE VI
Compounds of Structure VI

| Cpd. No. | Structure |
|---|---|
| 1-12 | 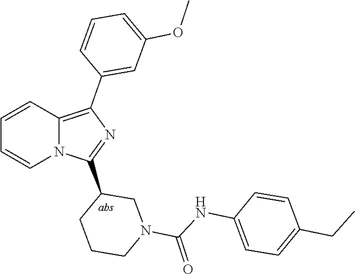 [SMILES: CCc1ccc(NC(=O)N2CCC[C@@H](c3nc(-c4cccc(OC)c4)c4ccccn34)C2)cc1] |
| 1-16 | 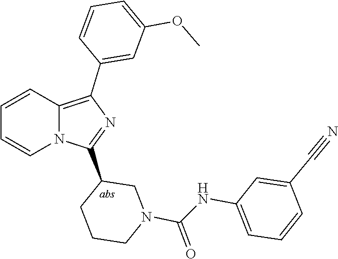 [SMILES: COc1cccc(-c2nc([C@@H]3CCCN(C(=O)Nc4cccc(C#N)c4)C3)n3ccccc23)c1] |
| 1-17 | 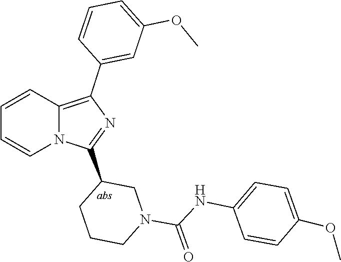 [SMILES: COc1ccc(NC(=O)N2CCC[C@@H](c3nc(-c4cccc(OC)c4)c4ccccn34)C2)cc1] |
| 1-18 | 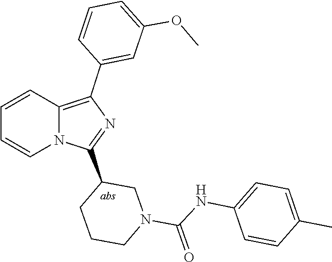 [SMILES: COc1cccc(-c2nc([C@@H]3CCCN(C(=O)Nc4ccc(C)cc4)C3)n3ccccc23)c1] |
| 1-22 | 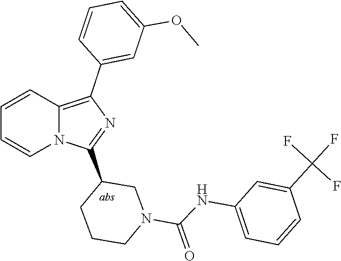 [SMILES: COc1cccc(-c2nc([C@@H]3CCCN(C(=O)Nc4cccc(C(F)(F)F)c4)C3)n3ccccc23)c1] |
| 1-23 | 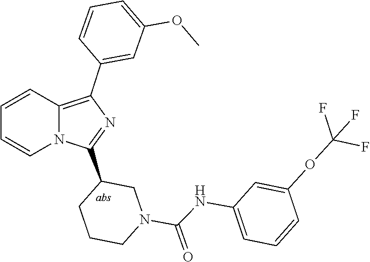 [SMILES: COc1cccc(-c2nc([C@@H]3CCCN(C(=O)Nc4cccc(OC(F)(F)F)c4)C3)n3ccccc23)c1] |
| 1-24 | 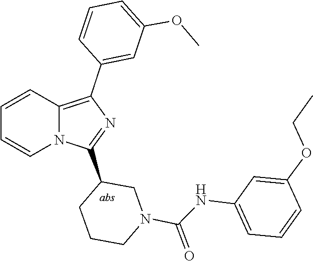 [SMILES: CCOc1cccc(NC(=O)N2CCC[C@@H](c3nc(-c4cccc(OC)c4)c4ccccn34)C2)c1] |
| 1-25 | 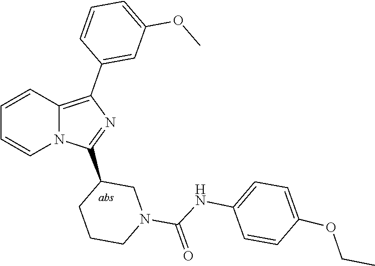 [SMILES: CCOc1ccc(NC(=O)N2CCC[C@@H](c3nc(-c4cccc(OC)c4)c4ccccn34)C2)cc1] |
| 1-31 | 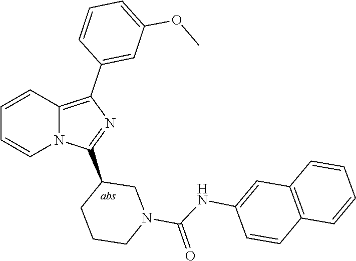 [SMILES: COc1cccc(-c2nc([C@@H]3CCCN(C(=O)Nc4ccc5ccccc5c4)C3)n3ccccc23)c1] |
| 3-1 | 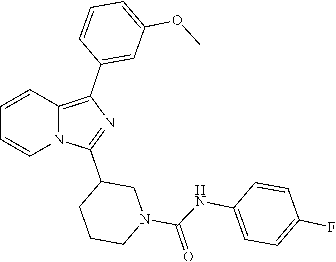 [SMILES: COc1cccc(-c2nc(C3CCCN(C(=O)Nc4ccc(F)cc4)C3)n3ccccc23)c1] |
| 3-4 | 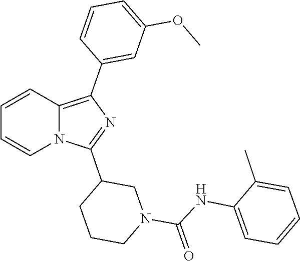 [SMILES: COc1cccc(-c2nc(C3CCCN(C(=O)Nc4ccccc4C)C3)n3ccccc23)c1] |
| 3-5 | 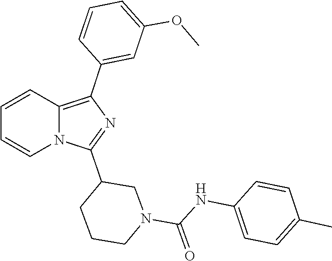 [SMILES: COc1cccc(-c2nc(C3CCCN(C(=O)Nc4ccc(C)cc4)C3)n3ccccc23)c1] |
| 3-6 | 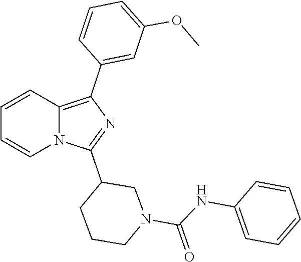 [SMILES: COc1cccc(-c2nc(C3CCCN(C(=O)Nc4ccccc4)C3)n3ccccc23)c1] |
| 3-7 | 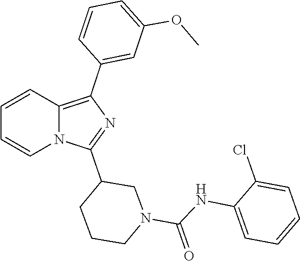 [SMILES: COc1cccc(-c2nc(C3CCCN(C(=O)Nc4ccccc4Cl)C3)n3ccccc23)c1] |
| 3-8 | 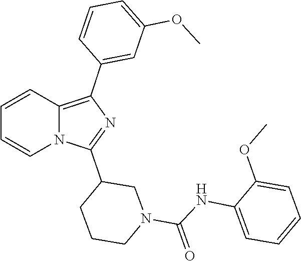 [SMILES: COc1cccc(-c2nc(C3CCCN(C(=O)Nc4ccccc4OC)C3)n3ccccc23)c1] |
| 3-15 | 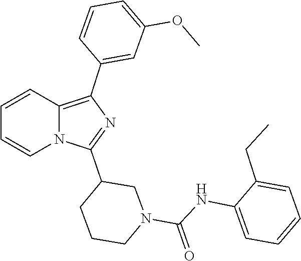 [SMILES: CCc1ccccc1NC(=O)N1CCCC(c2nc(-c3cccc(OC)c3)c3ccccn23)C1] |
| 3-17 | 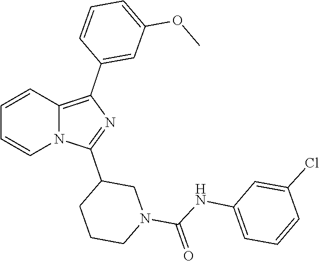 [SMILES: COc1cccc(-c2nc(C3CCCN(C(=O)Nc4cccc(Cl)c4)C3)n3ccccc23)c1] |
| 3-18 | 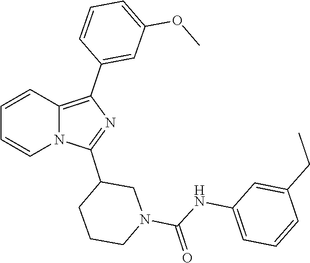 [SMILES: CCc1cccc(NC(=O)N2CCCC(c3nc(-c4cccc(OC)c4)c4ccccn34)C2)c1] |
| 3-19 | 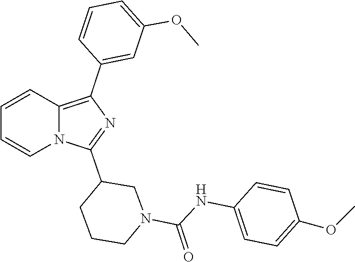 [SMILES: COc1ccc(NC(=O)N2CCCC(c3nc(-c4cccc(OC)c4)c4ccccn34)C2)cc1] |
| 3-20 | 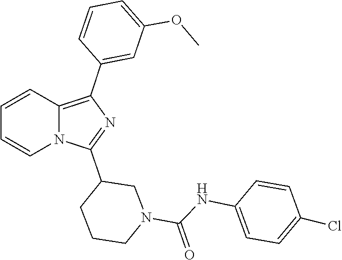 [SMILES: COc1cccc(-c2nc(C3CCCN(C(=O)Nc4ccc(Cl)cc4)C3)n3ccccc23)c1] |
| 3-21 | 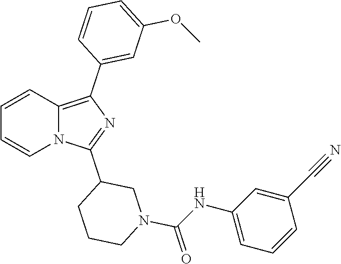 [SMILES: COc1cccc(-c2nc(C3CCCN(C(=O)Nc4cccc(C#N)c4)C3)n3ccccc23)c1] |
| 3-22 | 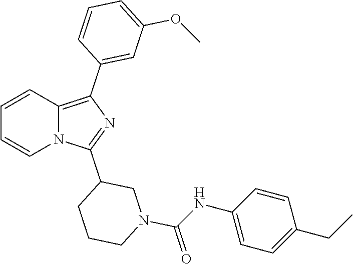 [SMILES: CCc1ccc(NC(=O)N2CCCC(c3nc(-c4cccc(OC)c4)c4ccccn34)C2)cc1] |
| 3-26 | 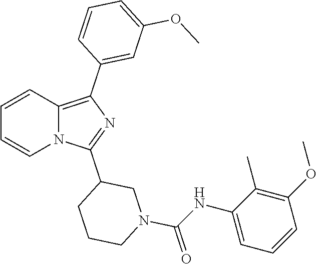 [SMILES: COc1cccc(-c2nc(C3CCCN(C(=O)Nc4cccc(OC)c4C)C3)n3ccccc23)c1] |
| 3-27 | 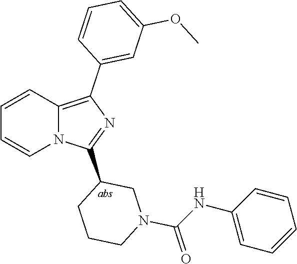 [SMILES: COc1cccc(-c2nc([C@@H]3CCCN(C(=O)Nc4ccccc4)C3)n3ccccc23)c1] |
| 3-28 | 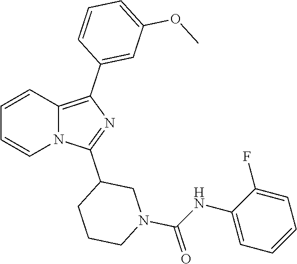 [SMILES: COc1cccc(-c2nc(C3CCCN(C(=O)Nc4ccccc4F)C3)n3ccccc23)c1] |
| 3-29 | 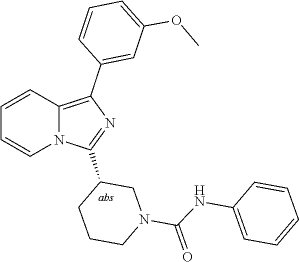 [SMILES: COc1cccc(-c2nc([C@H]3CCCN(C(=O)Nc4ccccc4)C3)n3ccccc23)c1] |
| 4-2 | 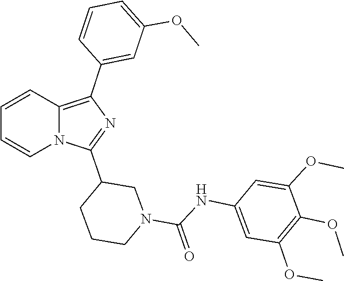 [SMILES: COc1cccc(-c2nc(C3CCCN(C(=O)Nc4cc(OC)c(OC)c(OC)c4)C3)n3ccccc23)c1] |
| 5-10 | 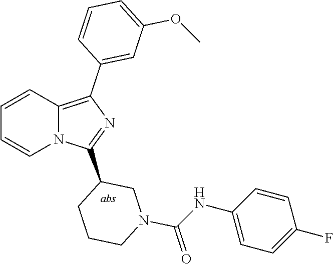 [SMILES: COc1cccc(-c2nc([C@@H]3CCCN(C(=O)Nc4ccc(F)cc4)C3)n3ccccc23)c1] |
| 5-11 | 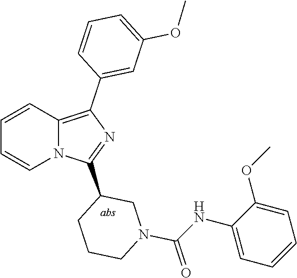 [SMILES: COc1cccc(-c2nc([C@@H]3CCCN(C(=O)Nc4ccccc4OC)C3)n3ccccc23)c1] |
| 5-12 | 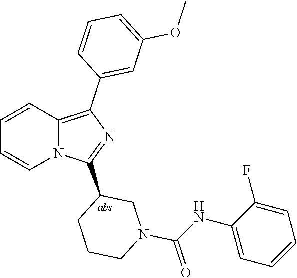 [SMILES: COc1cccc(-c2nc([C@@H]3CCCN(C(=O)Nc4ccccc4F)C3)n3ccccc23)c1] |
| 5-13 | 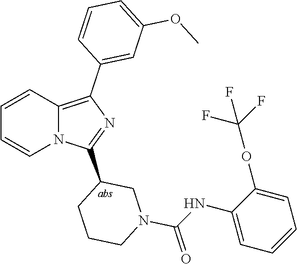 [SMILES: COc1cccc(-c2nc([C@@H]3CCCN(C(=O)Nc4ccccc4OC(F)(F)F)C3)n3ccccc23)c1] |
| 5-14 | 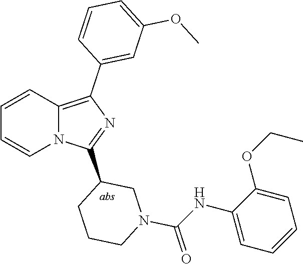 [SMILES: CCOc1ccccc1NC(=O)N1CCC[C@@H](c2nc(-c3cccc(OC)c3)c3ccccn23)C1] |
| 5-15 | 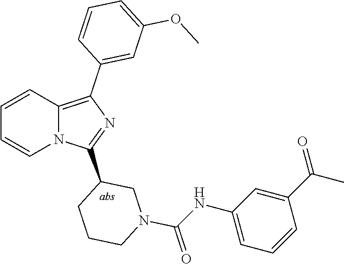 [SMILES: COc1cccc(-c2nc([C@@H]3CCCN(C(=O)Nc4cccc(C(C)=O)c4)C3)n3ccccc23)c1] |
| 5-21 | 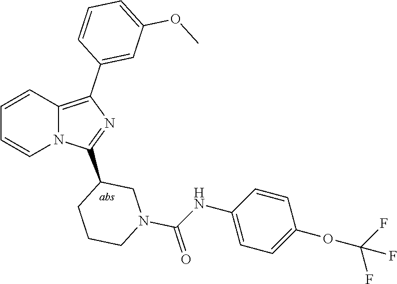 [SMILES: COc1cccc(-c2nc([C@@H]3CCCN(C(=O)Nc4ccc(OC(F)(F)F)cc4)C3)n3ccccc23)c1] |
| 5-22 | 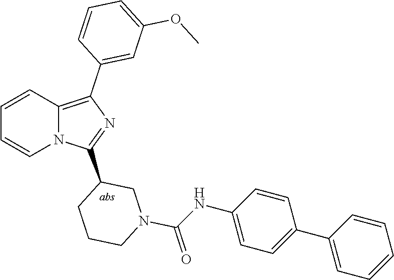 [SMILES: COc1cccc(-c2nc([C@@H]3CCCN(C(=O)Nc4ccc(-c5ccccc5)cc4)C3)n3ccccc23)c1] |
| 5-26 | 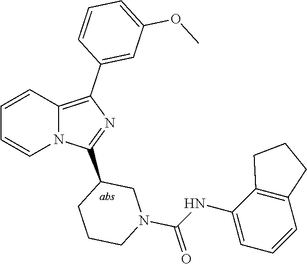 [SMILES: COc1cccc(-c2nc([C@@H]3CCCN(C(=O)Nc4cccc5c4CCC5)C3)n3ccccc23)c1] |
| 5-27 | 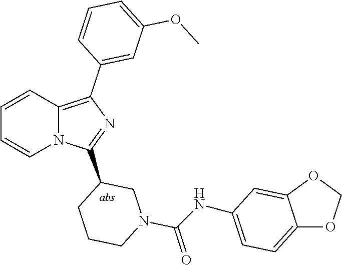 [SMILES: COc1cccc(-c2nc([C@@H]3CCCN(C(=O)Nc4ccc5c(c4)OCO5)C3)n3ccccc23)c1] |
| 5-29 | 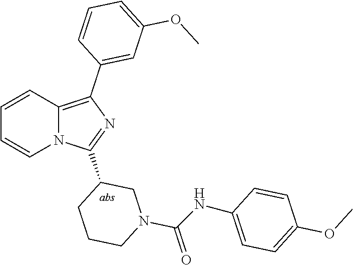 [SMILES: COc1ccc(NC(=O)N2CCC[C@H](c3nc(-c4cccc(OC)c4)c4ccccn34)C2)cc1] |
| 5-34 | 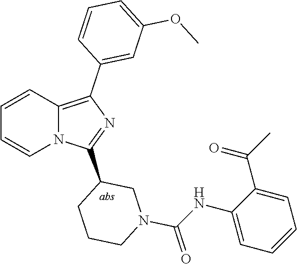 [SMILES: COc1cccc(-c2nc([C@@H]3CCCN(C(=O)Nc4ccccc4C(C)=O)C3)n3ccccc23)c1] |
| 5-35 | 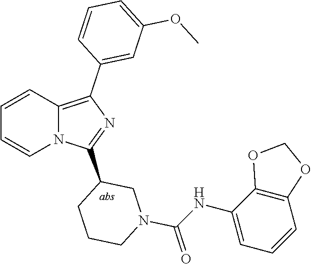 [SMILES: COc1cccc(-c2nc([C@@H]3CCCN(C(=O)Nc4cccc5c4OCO5)C3)n3ccccc23)c1] |
| 5-39 | 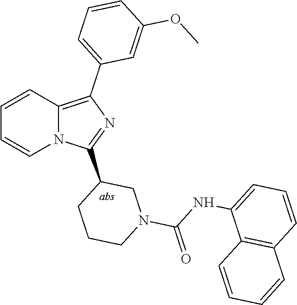 [SMILES: COc1cccc(-c2nc([C@@H]3CCCN(C(=O)Nc4cccc5ccccc45)C3)n3ccccc23)c1] |
| 5-47 | 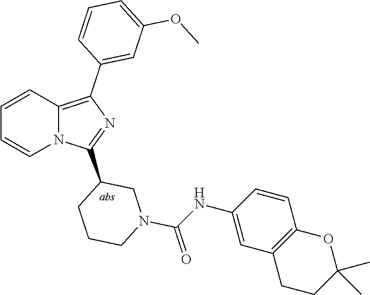 [SMILES: COc1cccc(-c2nc([C@@H]3CCCN(C(=O)Nc4ccc5c(c4)CCC(C)(C)O5)C3)n3ccccc23)c1] |
| 5-57 | 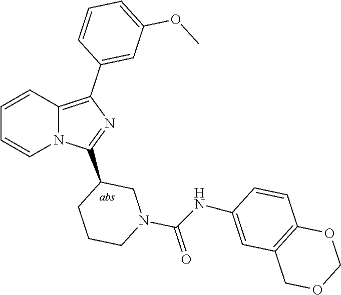 [SMILES: COc1cccc(-c2nc([C@@H]3CCCN(C(=O)Nc4ccc5c(c4)COCO5)C3)n3ccccc23)c1] |

In some embodiments, compounds having activity as modulators of the Mas-related G-protein coupled receptor D are provided, the compounds having the following structure (VII):

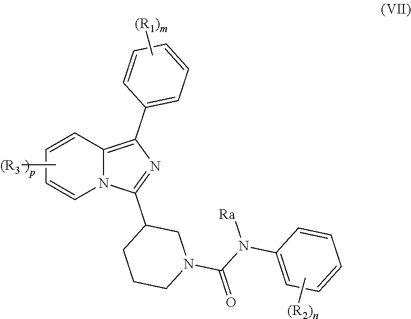

or a pharmaceutically acceptable salt, isomer, hydrate, solvate, or isotope thereof, wherein:
R₁ is hydroxyl, halo, cyano, alkyl, haloalkyl, hydroxylalkyl, C₂-C₆ alkoxy, haloalkoxy, aminyl, aminylalkyl, cyanoalkyl, alkoxyalkyl, alkylcarbonyl, alkoxycarbonyl, aminylcarbonyl, alkylsulfonyl, cycloalkylalkyl, aryl, aralkyl, or arylsulfonyl;
R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
Ra is H or alkyl;
m is 0, 1, or 2;
n is 0, 1, 2, or 3; and
p is 0, 1, 2, or 3.

In some embodiments, compounds of structure (VII) are provided wherein R₁ is hydroxyl, halo, cyano, alkyl, haloalkyl, hydroxylalkyl, C₂-C₆ alkoxy, haloalkoxy, aminyl, aminylalkyl, cyanoalkyl, alkoxyalkyl, alkylcarbonyl, alkoxycarbonyl, aminylcarbonyl, or alkylsulfonyl. In other embodiments, compounds of structure (VII) are provided wherein R₁ is cycloalkylalkyl, aryl, aralkyl, or arylsulfonyl. In certain embodiments, compounds of structure (VII) are provided wherein R₁ is selected from:

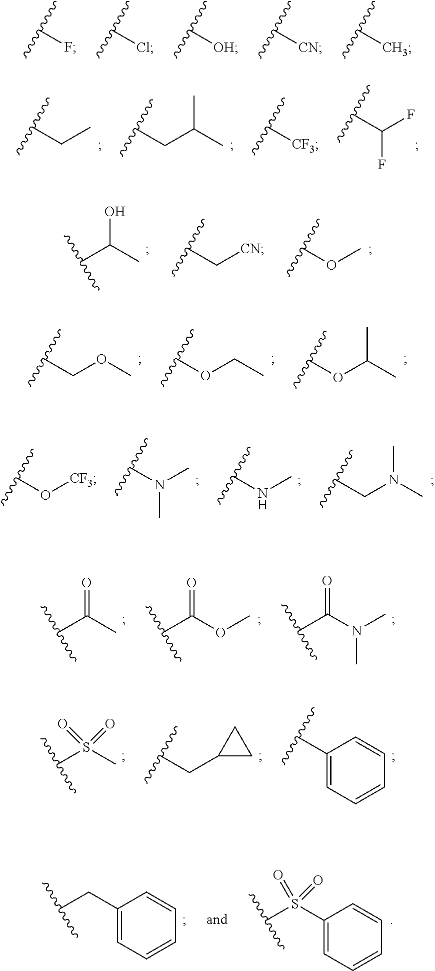

In specific embodiments, compounds of structure (VII) are provided wherein R₁ is selected from:

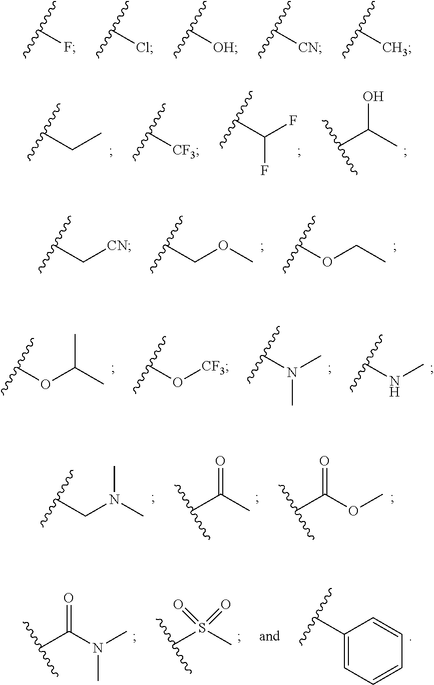

In some embodiments, compounds of structure (VII) are provided wherein R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, or alkylsufonylalkyl. In some embodiments, compounds of structure (VII) are provided wherein R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, or alkylsulfonyl. In other embodiments, compounds of structure (VII) are provided wherein R₂ is aryl, heterocyclyl, or alkylheteroaryl. In certain embodiments, compounds of structure (VII) are provided wherein R₂ is selected from:

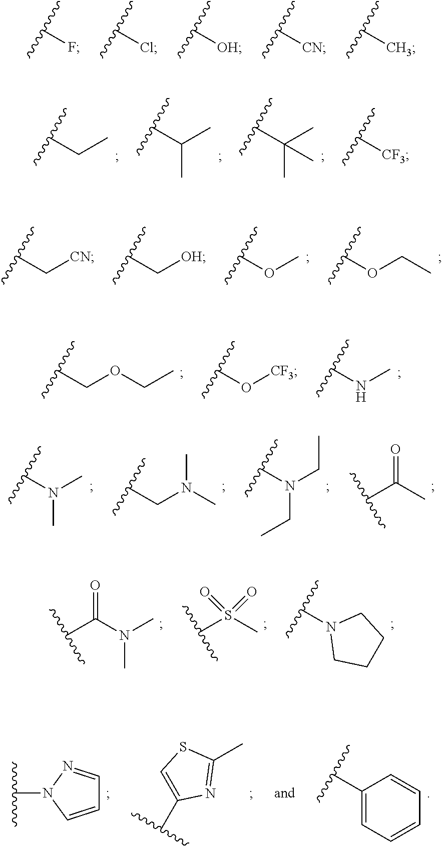

In specific embodiments, compounds of structure (VII) are provided wherein R₂ is selected from halo and aminyl. In specific embodiments, compounds of structure (VII) are provided wherein R₂ is selected from:

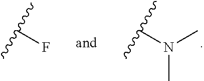

In some embodiments, compounds of structure (VII) are provided wherein R₃ is alkyl. In other embodiments, compounds of structure (VII) are provided wherein R₃ is alkoxy.

In some embodiments, compounds of structure (VII) are provided wherein Ra is H. In other embodiments, compounds of structure (VII) are provided wherein Ra is alkyl.

In some embodiments, compounds of structure (VII) are provided wherein m is 0. In other embodiments, compounds of structure (VII) are provided wherein m is 1. In additional embodiments, compounds of structure (VII) are provided wherein m is 2.

In some embodiments, compounds of structure (VII) are provided wherein n is 0. In other embodiments, compounds of structure (VII) are provided wherein n is 1. In additional embodiments, compounds of structure (VII) are provided wherein n is 2. In further embodiments, compounds of structure (VII) are provided wherein n is 3.

In some embodiments, compounds of structure (VII) are provided wherein p is 0. In other embodiments, compounds of structure (VII) are provided wherein p is 1. In additional embodiments, compounds of structure (VII) are provided wherein p is 2. In further embodiments, compounds of structure (VII) are provided wherein p is 3.

Representative compounds of structure (VII) include any one of the compounds listed in Table VII below, as well as a pharmaceutically acceptable isomer, racemate, hydrate, solvate, isotope, or salt thereof.

TABLE VII
Compounds of Structure VII

| Cpd. No. | Structure |
|---|---|
| 10-1 | 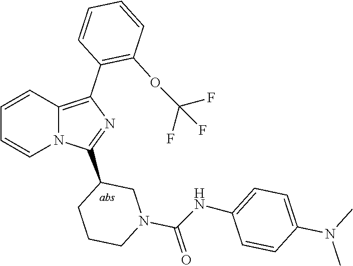 [SMILES: CN(C)c1ccc(NC(=O)N2CCC[C@@H](c3nc(-c4ccccc4OC(F)(F)F)c4ccccn34)C2)cc1] |
| 10-2 | 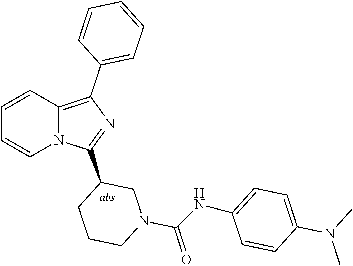 [SMILES: CN(C)c1ccc(NC(=O)N2CCC[C@@H](c3nc(-c4ccccc4)c4ccccn34)C2)cc1] |
| 10-3 | 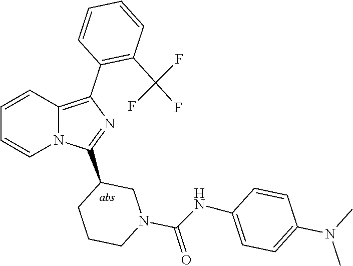 [SMILES: CN(C)c1ccc(NC(=O)N2CCC[C@@H](c3nc(-c4ccccc4C(F)(F)F)c4ccccn34)C2)cc1] |
| 10-4 | 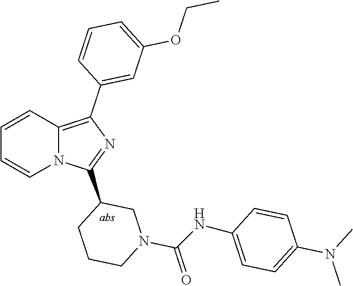 [SMILES: CCOc1cccc(-c2nc([C@@H]3CCCN(C(=O)Nc4ccc(N(C)C)cc4)C3)n3ccccc23)c1] |
| 10-5 | 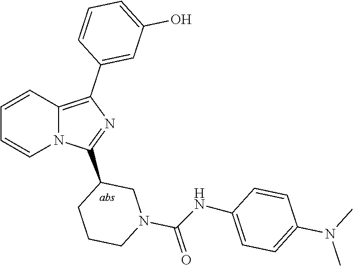 [SMILES: CN(C)c1ccc(NC(=O)N2CCC[C@@H](c3nc(-c4cccc(O)c4)c4ccccn34)C2)cc1] |
| 10-6 | 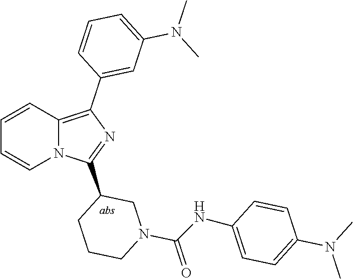 [SMILES: CN(C)c1ccc(NC(=O)N2CCC[C@@H](c3nc(-c4cccc(N(C)C)c4)c4ccccn34)C2)cc1] |
| 10-7 | 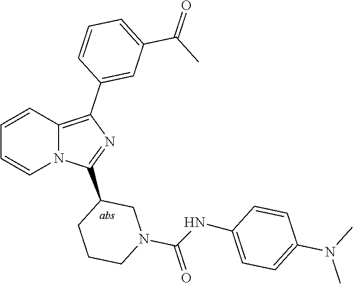 [SMILES: CC(=O)c1cccc(-c2nc([C@@H]3CCCN(C(=O)Nc4ccc(N(C)C)cc4)C3)n3ccccc23)c1] |
| 10-8 | 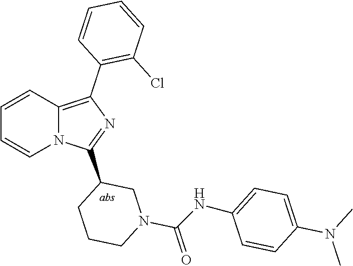 [SMILES: CN(C)c1ccc(NC(=O)N2CCC[C@@H](c3nc(-c4ccccc4Cl)c4ccccn34)C2)cc1] |
| 10-9 | 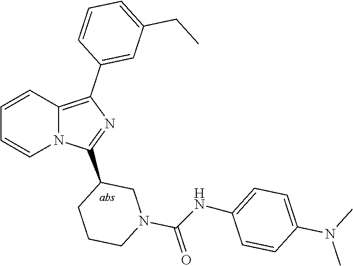 [SMILES: CCc1cccc(-c2nc([C@@H]3CCCN(C(=O)Nc4ccc(N(C)C)cc4)C3)n3ccccc23)c1] |
| 10-10 | 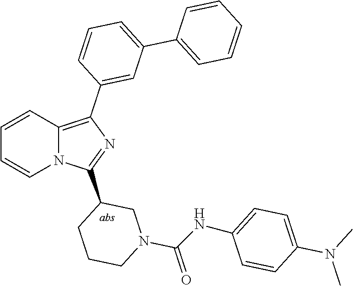 [SMILES: CN(C)c1ccc(NC(=O)N2CCC[C@@H](c3nc(-c4cccc(-c5ccccc5)c4)c4ccccn34)C2)cc1] |
| 10-11 | 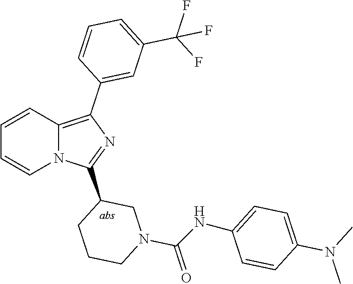 [SMILES: CN(C)c1ccc(NC(=O)N2CCC[C@@H](c3nc(-c4cccc(C(F)(F)F)c4)c4ccccn34)C2)cc1] |
| 10-12 | 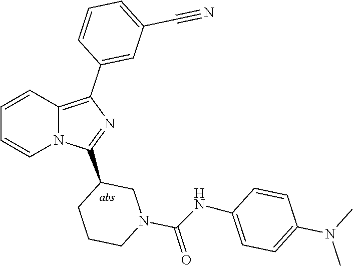 [SMILES: CN(C)c1ccc(NC(=O)N2CCC[C@@H](c3nc(-c4cccc(C#N)c4)c4ccccn34)C2)cc1] |
| 10-13 | 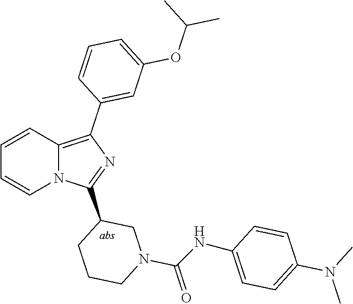 [SMILES: CC(C)Oc1cccc(-c2nc([C@@H]3CCCN(C(=O)Nc4ccc(N(C)C)cc4)C3)n3ccccc23)c1] |
| 10-14 | 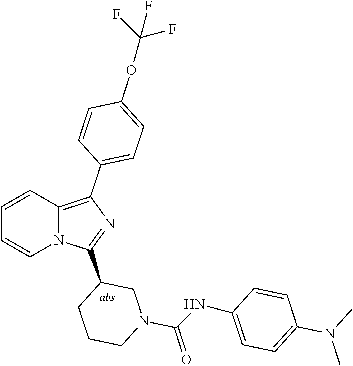 [SMILES: CN(C)c1ccc(NC(=O)N2CCC[C@@H](c3nc(-c4ccc(OC(F)(F)F)cc4)c4ccccn34)C2)cc1] |
| 10-15 | 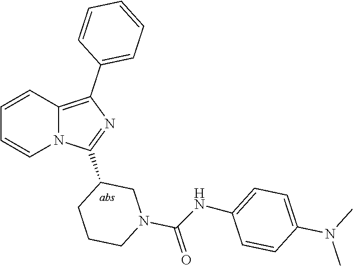 [SMILES: CN(C)c1ccc(NC(=O)N2CCC[C@H](c3nc(-c4ccccc4)c4ccccn34)C2)cc1] |
| 10-16 | 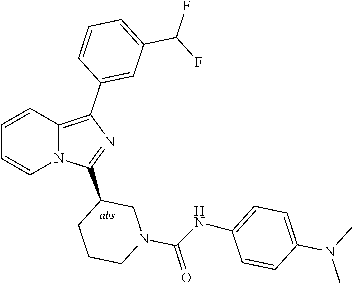 [SMILES: CN(C)c1ccc(NC(=O)N2CCC[C@@H](c3nc(-c4cccc(C(F)F)c4)c4ccccn34)C2)cc1] |
| 10-18 | 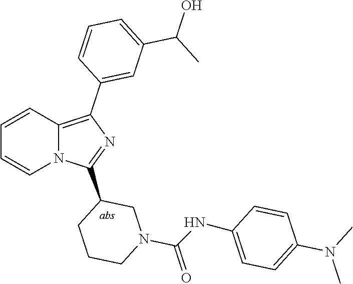 [SMILES: CC(O)c1cccc(-c2nc([C@@H]3CCCN(C(=O)Nc4ccc(N(C)C)cc4)C3)n3ccccc23)c1] |
| 10-19 | 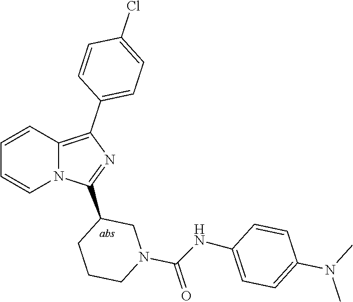 [SMILES: CN(C)c1ccc(NC(=O)N2CCC[C@@H](c3nc(-c4ccc(Cl)cc4)c4ccccn34)C2)cc1] |
| 10-20 | 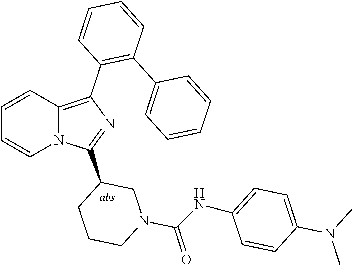 [SMILES: CN(C)c1ccc(NC(=O)N2CCC[C@@H](c3nc(-c4ccccc4-c4ccccc4)c4ccccn34)C2)cc1] |
| 10-21 | 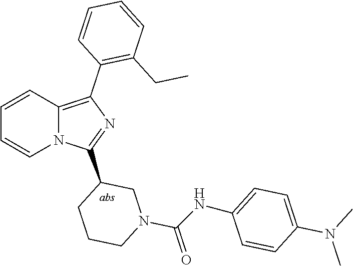 [SMILES: CCc1ccccc1-c1nc([C@@H]2CCCN(C(=O)Nc3ccc(N(C)C)cc3)C2)n2ccccc12] |
| 10-22 | 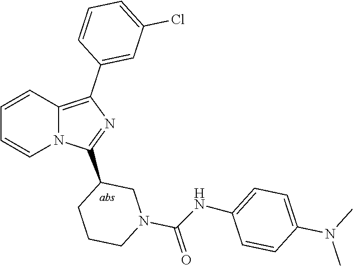 [SMILES: CN(C)c1ccc(NC(=O)N2CCC[C@@H](c3nc(-c4cccc(Cl)c4)c4ccccn34)C2)cc1] |
| 10-23 | 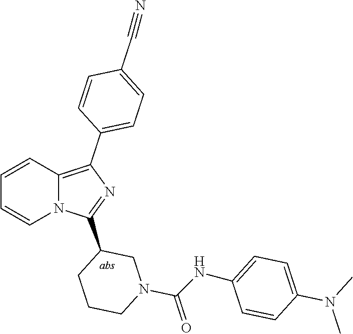 [SMILES: CN(C)c1ccc(NC(=O)N2CCC[C@@H](c3nc(-c4ccc(C#N)cc4)c4ccccn34)C2)cc1] |
| 10-24 | 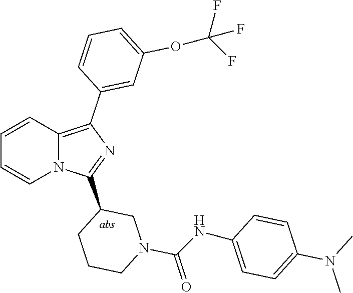 [SMILES: CN(C)c1ccc(NC(=O)N2CCC[C@@H](c3nc(-c4cccc(OC(F)(F)F)c4)c4ccccn34)C2)cc1] |
| 10-28 | 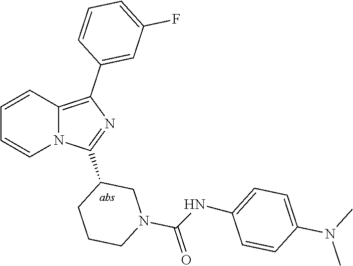 [SMILES: CN(C)c1ccc(NC(=O)N2CCC[C@H](c3nc(-c4cccc(F)c4)c4ccccn34)C2)cc1] |
| 10-29 | 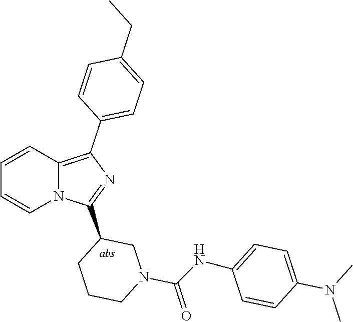 [SMILES: CCc1ccc(-c2nc([C@@H]3CCCN(C(=O)Nc4ccc(N(C)C)cc4)C3)n3ccccc23)cc1] |
| 10-30 | 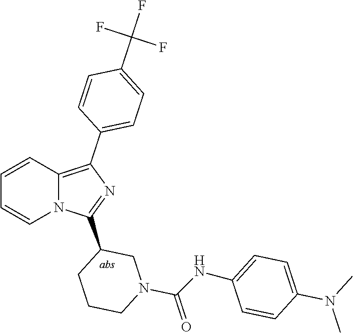 [SMILES: CN(C)c1ccc(NC(=O)N2CCC[C@@H](c3nc(-c4ccc(C(F)(F)F)cc4)c4ccccn34)C2)cc1] |
| 10-32 | 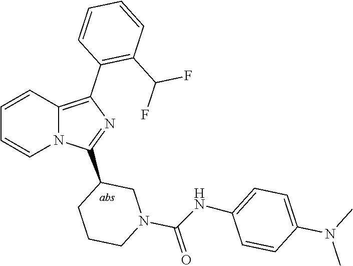 [SMILES: CN(C)c1ccc(NC(=O)N2CCC[C@@H](c3nc(-c4ccccc4C(F)F)c4ccccn34)C2)cc1] |
| 10-33 | 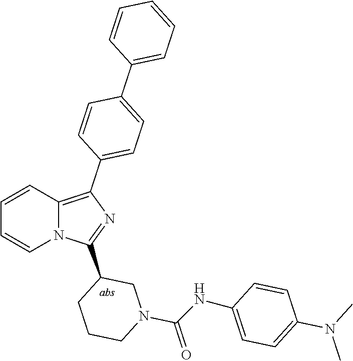 [SMILES: CN(C)c1ccc(NC(=O)N2CCC[C@@H](c3nc(-c4ccc(-c5ccccc5)cc4)c4ccccn34)C2)cc1] |
| 10-35 | 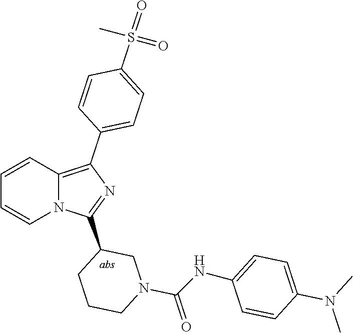 [SMILES: CN(C)c1ccc(NC(=O)N2CCC[C@@H](c3nc(-c4ccc(S(C)(=O)=O)cc4)c4ccccn34)C2)cc1] |
| 10-36 | 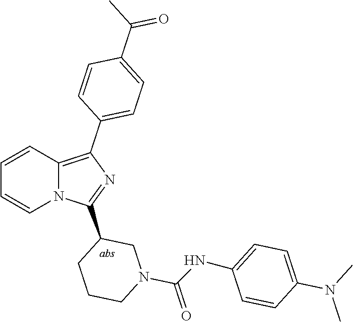 [SMILES: CC(=O)c1ccc(-c2nc([C@@H]3CCCN(C(=O)Nc4ccc(N(C)C)cc4)C3)n3ccccc23)cc1] |
| 10-40 | 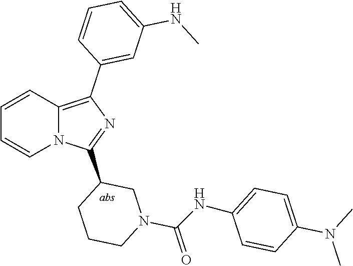 [SMILES: CNc1cccc(-c2nc([C@@H]3CCCN(C(=O)Nc4ccc(N(C)C)cc4)C3)n3ccccc23)c1] |
| 10-42 | 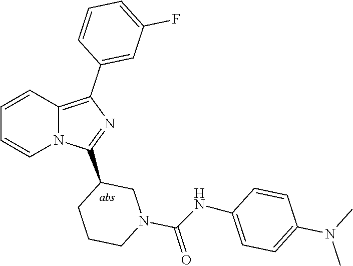 [SMILES: CN(C)c1ccc(NC(=O)N2CCC[C@@H](c3nc(-c4cccc(F)c4)c4ccccn34)C2)cc1] |
| 10-46 | 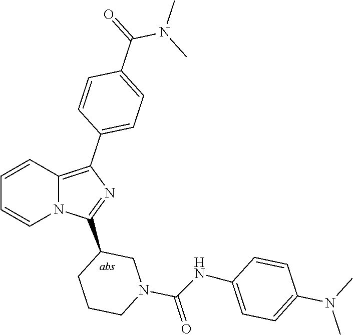 [SMILES: CN(C)C(=O)c1ccc(-c2nc([C@@H]3CCCN(C(=O)Nc4ccc(N(C)C)cc4)C3)n3ccccc23)cc1] |
| 10-49 | 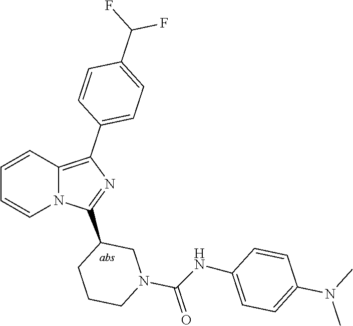 [SMILES: CN(C)c1ccc(NC(=O)N2CCC[C@@H](c3nc(-c4ccc(C(F)F)cc4)c4ccccn34)C2)cc1] |
| 10-59 | 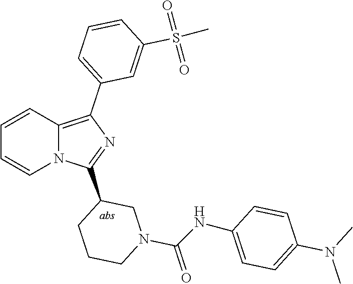 [SMILES: CN(C)c1ccc(NC(=O)N2CCC[C@@H](c3nc(-c4cccc(S(C)(=O)=O)c4)c4ccccn34)C2)cc1] |
| 10-64 | 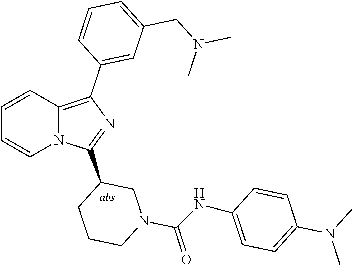 [SMILES: CN(C)Cc1cccc(-c2nc([C@@H]3CCCN(C(=O)Nc4ccc(N(C)C)cc4)C3)n3ccccc23)c1] |
| 10-65 | 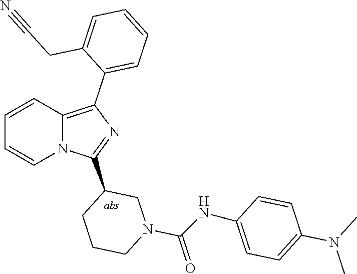 [SMILES: CN(C)c1ccc(NC(=O)N2CCC[C@@H](c3nc(-c4ccccc4CC#N)c4ccccn34)C2)cc1] |
| 10-66 | 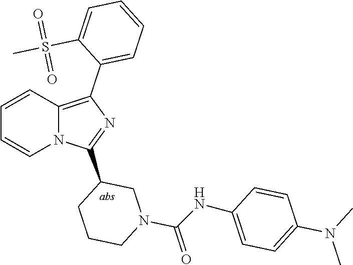 [SMILES: CN(C)c1ccc(NC(=O)N2CCC[C@@H](c3nc(-c4ccccc4S(C)(=O)=O)c4ccccn34)C2)cc1] |
| 10-67 | 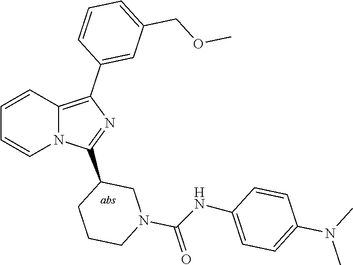 [SMILES: COCc1cccc(-c2nc([C@@H]3CCCN(C(=O)Nc4ccc(N(C)C)cc4)C3)n3ccccc23)c1] |
| 10-70 | 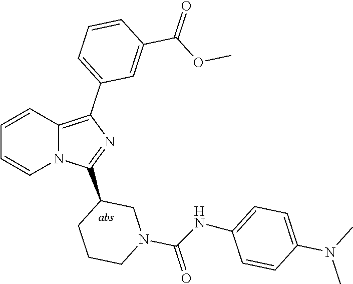 [SMILES: COC(=O)c1cccc(-c2nc([C@@H]3CCCN(C(=O)Nc4ccc(N(C)C)cc4)C3)n3ccccc23)c1] |
| 10-71 | 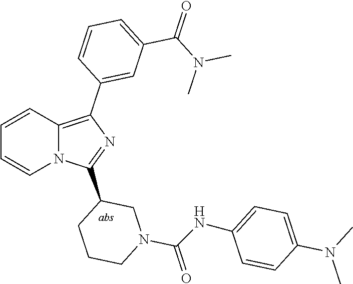 [SMILES: CN(C)C(=O)c1cccc(-c2nc([C@@H]3CCCN(C(=O)Nc4ccc(N(C)C)cc4)C3)n3ccccc23)c1] |
| 11-1 | 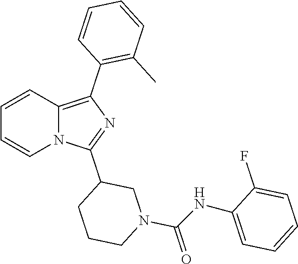 [SMILES: Cc1ccccc1-c1nc(C2CCCN(C(=O)Nc3ccccc3F)C2)n2ccccc12] |
| 11-2 | 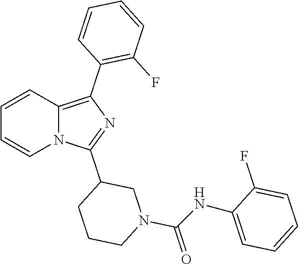 [SMILES: O=C(Nc1ccccc1F)N1CCCC(c2nc(-c3ccccc3F)c3ccccn23)C1] |
| 11-4 | 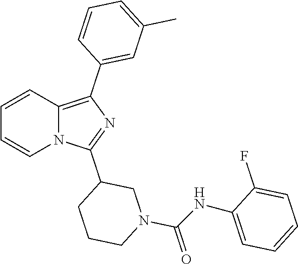 [SMILES: Cc1cccc(-c2nc(C3CCCN(C(=O)Nc4ccccc4F)C3)n3ccccc23)c1] |
| 11-5 | 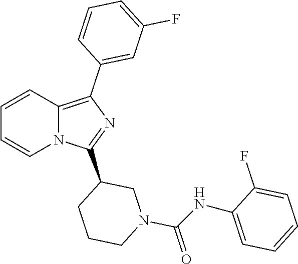 [SMILES: O=C(Nc1ccccc1F)N1CCC[C@@H](c2nc(-c3cccc(F)c3)c3ccccn23)C1] |
| 11-7 | 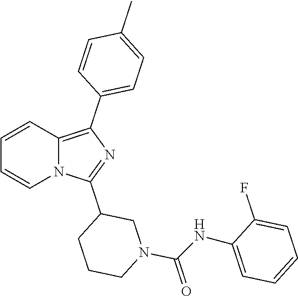 [SMILES: Cc1ccc(-c2nc(C3CCCN(C(=O)Nc4ccccc4F)C3)n3ccccc23)cc1] |
| 11-8 | 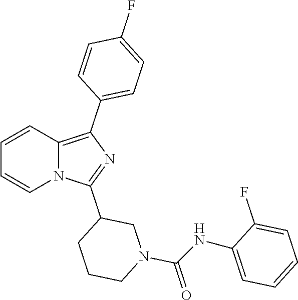 [SMILES: O=C(Nc1ccccc1F)N1CCCC(c2nc(-c3ccc(F)cc3)c3ccccn23)C1] |

In some embodiments, compounds having activity as modulators of the Mas-related G-protein coupled receptor D are provided, the compounds having the following structure (VIII):

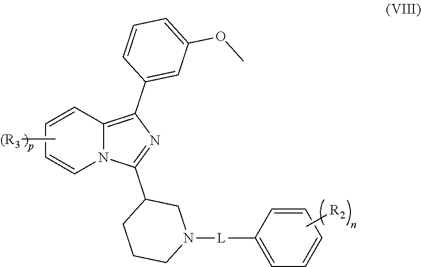

or a pharmaceutically acceptable salt, isomer, hydrate, solvate, or isotope thereof, wherein:
L is a linker having the structure —C(O)—, —C(O)-alkyl-, —C(O)—NRa-, —C(O)—NRa-alkyl-, —S(O)₂—, or —S(O)₂-alkyl-;
R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
Ra is H or alkyl;
n is 0, 1, 2, or 3; and
p is 1, 2, or 3.

In some embodiments, compounds of structure (VIII) are provided wherein L is a linker having the structure —C(O)—. In other embodiments, compounds of structure (VIII) are provided wherein L is a linker having the structure —C(O)-alkyl-. In additional embodiments, compounds of structure (VIII) are provided wherein L is a linker having the structure —C(O)—NRa-. In further embodiments, compounds of structure (VIII) are provided wherein L is a linker having the structure —C(O)—NRa-alkyl-. In yet other embodiments, compounds of structure (VIII) are provided wherein L is a linker having the structure —S(O)₂—. In some embodiments, compounds of structure (VIII) are provided wherein L is a linker having the structure —S(O)₂-alkyl-.

In some embodiments, compounds of structure (VIII) are provided wherein R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, or alkylsufonylalkyl. In some embodiments, compounds of structure (VIII) are provided wherein R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, or alkylsulfonyl. In other embodiments, compounds of structure (VIII) are provided wherein R₂ is aryl, heterocyclyl, or alkylheteroaryl. In certain embodiments, compounds of structure (VIII) are provided wherein R₂ is selected from:

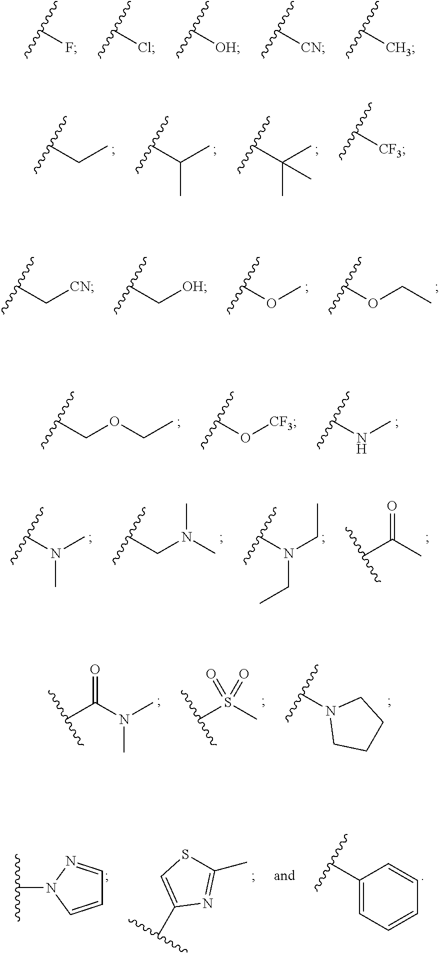

In specific embodiments, compounds of structure (VIII) are provided wherein R₂ is selected from halo and alkoxy. In specific embodiments, compounds of structure (VIII) are provided wherein R₂ is selected from:

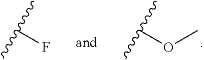

In some embodiments, compounds of structure (VIII) are provided wherein R₃ is alkyl. In other embodiments, compounds of structure (VIII) are provided wherein R₃ is alkoxy.

In some embodiments, compounds of structure (VIII) are provided wherein Ra is H. In other embodiments, compounds of structure (VIII) are provided wherein Ra is alkyl.

In some embodiments, compounds of structure (VIII) are provided wherein n is 0. In other embodiments, compounds of structure (VIII) are provided wherein n is 1. In additional embodiments, compounds of structure (VIII) are provided wherein n is 2. In further embodiments, compounds of structure (VIII) are provided wherein n is 3.

In other embodiments, compounds of structure (VIII) are provided wherein p is 1. In additional embodiments, compounds of structure (VIII) are provided wherein p is 2. In further embodiments, compounds of structure (VIII) are provided wherein p is 3.

Representative compounds of structure (VIII) include any one of the compounds listed in Table VIII below, as well as a pharmaceutically acceptable isomer, racemate, hydrate, solvate, isotope, or salt thereof.

TABLE VIII
Compounds of Structure VIII

| Cpd. No. | Structure |
|---|---|
| 13-1 | 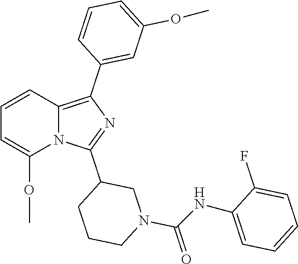 [SMILES: COc1cccc(-c2nc(C3CCCN(C(=O)Nc4ccccc4F)C3)n3c(OC)cccc23)c1] |
| 14-1 | 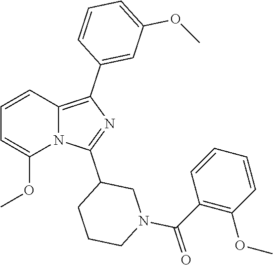 [SMILES: COc1cccc(-c2nc(C3CCCN(C(=O)c4ccccc4OC)C3)n3c(OC)cccc23)c1] |
| 14-2 | 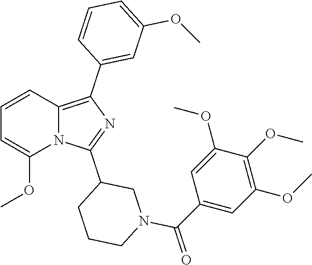 [SMILES: COc1cccc(-c2nc(C3CCCN(C(=O)c4cc(OC)c(OC)c(OC)c4)C3)n3c(OC)cccc23)c1] |
| 14-3 | 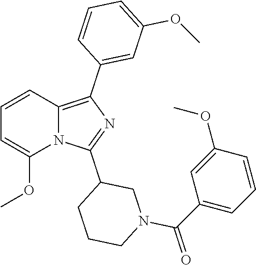 [SMILES: COc1cccc(C(=O)N2CCCC(c3nc(-c4cccc(OC)c4)c4cccc(OC)n34)C2)c1] |
| 14-4 | 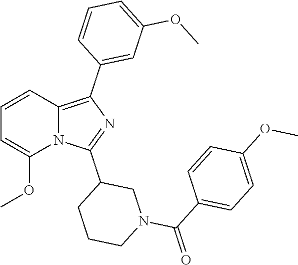 [SMILES: COc1ccc(C(=O)N2CCCC(c3nc(-c4cccc(OC)c4)c4cccc(OC)n34)C2)cc1] |
| 14-5 | 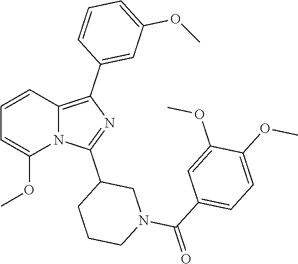 [SMILES: COc1cccc(-c2nc(C3CCCN(C(=O)c4ccc(OC)c(OC)c4)C3)n3c(OC)cccc23)c1] |
| 15-1 | 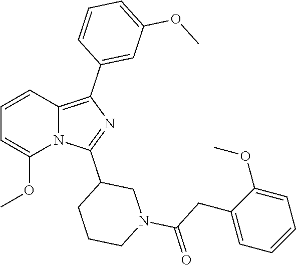 [SMILES: COc1cccc(-c2nc(C3CCCN(C(=O)Cc4ccccc4OC)C3)n3c(OC)cccc23)c1] |
| 17-1 | 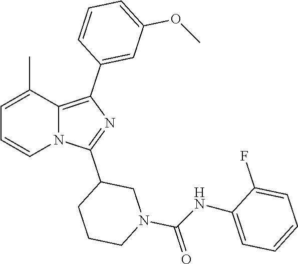 [SMILES: COc1cccc(-c2nc(C3CCCN(C(=O)Nc4ccccc4F)C3)n3cccc(C)c23)c1] |
| 17-2 | 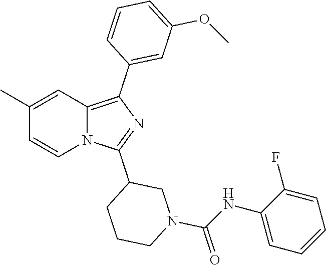 [SMILES: COc1cccc(-c2nc(C3CCCN(C(=O)Nc4ccccc4F)C3)n3ccc(C)cc23)c1] |
| 17-3 | 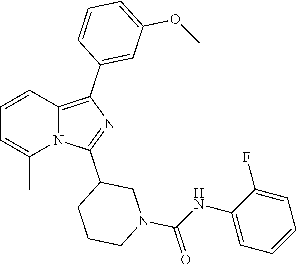 [SMILES: COc1cccc(-c2nc(C3CCCN(C(=O)Nc4ccccc4F)C3)n3c(C)cccc23)c1] |
| 18-1 | 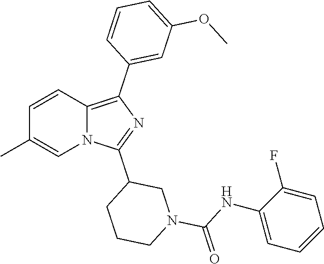 [SMILES: COc1cccc(-c2nc(C3CCCN(C(=O)Nc4ccccc4F)C3)n3cc(C)ccc23)c1] |
| 19-1 | 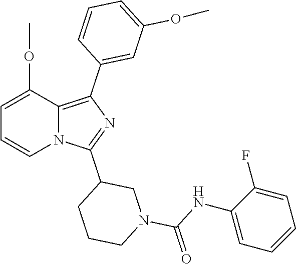 [SMILES: COc1cccc(-c2nc(C3CCCN(C(=O)Nc4ccccc4F)C3)n3cccc(OC)c23)c1] |
| 19-2 | 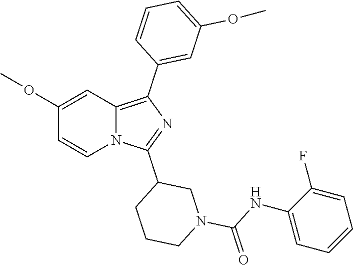 [SMILES: COc1cccc(-c2nc(C3CCCN(C(=O)Nc4ccccc4F)C3)n3ccc(OC)cc23)c1] |
| 19-3 | 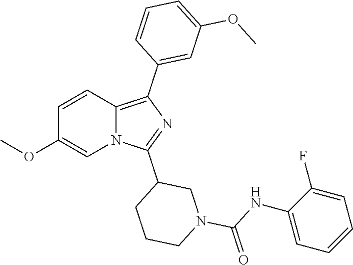 [SMILES: COc1cccc(-c2nc(C3CCCN(C(=O)Nc4ccccc4F)C3)n3cc(OC)ccc23)c1] |

In some embodiments, compounds having activity as modulators of the Mas-related G-protein coupled receptor D are provided, the compounds having the following structure (IX):

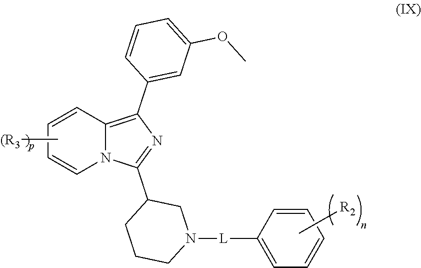

or a pharmaceutically acceptable salt, isomer, hydrate, solvate, or isotope thereof, wherein:
L is a linker having the structure —C(O)—, —C(O)-alkyl-, —S(O)₂—, or —S(O)₂-alkyl-;
each R₂ is individually hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl; and
R₃ is alkyl or alkoxy;
n is 0, 1, 2, or 3; and
p is 0, 1, 2, or 3.

In some embodiments, compounds of structure (IX) are provided wherein L is a linker having the structure —C(O)—. In other embodiments, compounds of structure (IX) are provided wherein L is a linker having the structure —C(O)-alkyl-. In additional embodiments, compounds of structure (IX) are provided wherein L is a linker having the structure —S(O)₂—. In further embodiments, compounds of structure (IX) are provided wherein L is a linker having the structure —S(O)₂-alkyl-.

In some embodiments, compounds of structure (IX) are provided wherein R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, or alkylsufonylalkyl. In some embodiments, compounds of structure (IX) are provided wherein R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, or alkylsulfonyl. In other embodiments, compounds of structure (IX) are provided wherein R₂ is aryl, heterocyclyl, or alkylheteroaryl. In certain embodiments, compounds of structure (IX) are provided wherein R₂ is selected from:

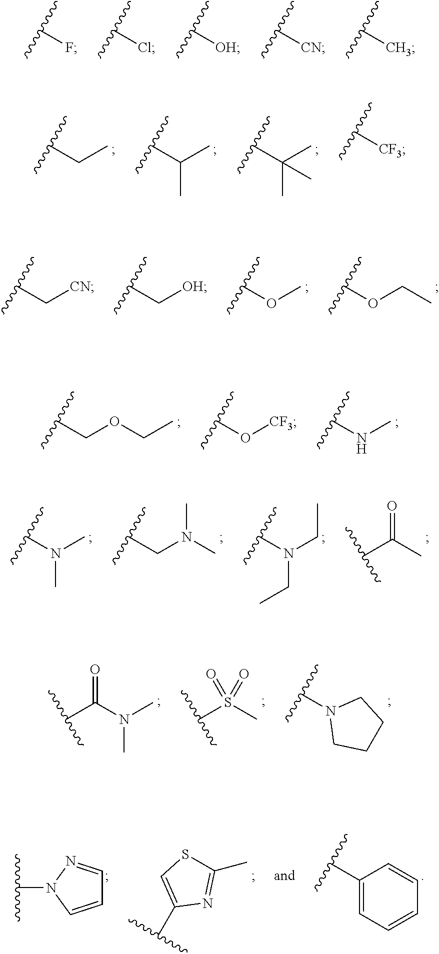

In specific embodiments, compounds of structure (IX) are provided wherein R₂ is selected from halo, alkyl, and alkoxy. In specific embodiments, compounds of structure (IX) are provided wherein R₂ is selected from:

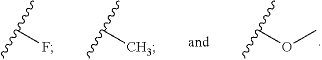

In some embodiments, compounds of structure (IX) are provided wherein R₃ is alkyl. In other embodiments, compounds of structure (IX) are provided wherein R₃ is alkoxy.

In some embodiments, compounds of structure (IX) are provided wherein n is 0. In other embodiments, compounds of structure (IX) are provided wherein n is 1. In additional embodiments, compounds of structure (IX) are provided wherein n is 2. In further embodiments, compounds of structure (IX) are provided wherein n is 3.

In some embodiments, compounds of structure (IX) are provided wherein p is 0. In other embodiments, compounds of structure (IX) are provided wherein p is 1. In additional embodiments, compounds of structure (IX) are provided wherein p is 2. In further embodiments, compounds of structure (IX) are provided wherein p is 3.

Representative compounds of structure (IX) include any one of the compounds listed in Table IX below, as well as a pharmaceutically acceptable isomer, racemate, hydrate, solvate, isotope, or salt thereof.

TABLE IX
Compounds of Structure IX

| Cpd. No. | Structure |
|---|---|
| 12-1 | 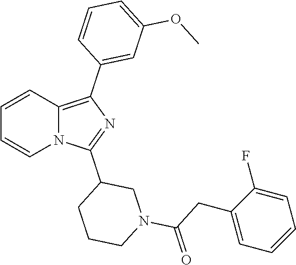 [SMILES: COc1cccc(-c2nc(C3CCCN(C(=O)Cc4ccccc4F)C3)n3ccccc23)c1] |
| 12-2 | 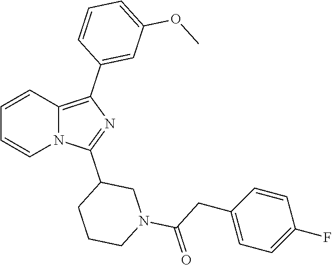 [SMILES: COc1cccc(-c2nc(C3CCCN(C(=O)Cc4ccc(F)cc4)C3)n3ccccc23)c1] |
| 12-3 | 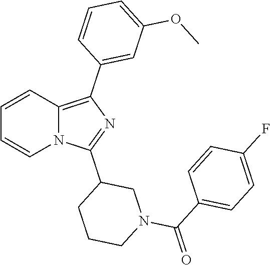 [SMILES: COc1cccc(-c2nc(C3CCCN(C(=O)c4ccc(F)cc4)C3)n3ccccc23)c1] |
| 12-4 | 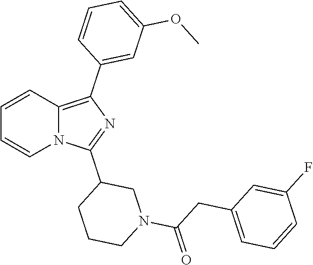 [SMILES: COc1cccc(-c2nc(C3CCCN(C(=O)Cc4cccc(F)c4)C3)n3ccccc23)c1] |
| 12-5 | 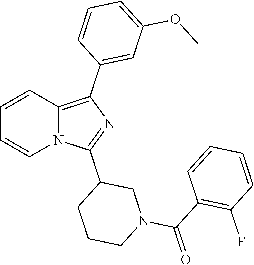 [SMILES: COc1cccc(-c2nc(C3CCCN(C(=O)c4ccccc4F)C3)n3ccccc23)c1] |
| 12-6 | 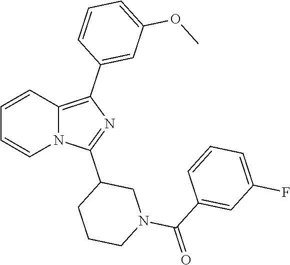 [SMILES: COc1cccc(-c2nc(C3CCCN(C(=O)c4cccc(F)c4)C3)n3ccccc23)c1] |
| 12-7 | 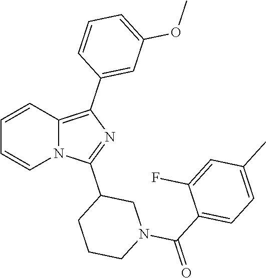 [SMILES: COc1cccc(-c2nc(C3CCCN(C(=O)c4ccc(C)cc4F)C3)n3ccccc23)c1] |
| 12-8 | 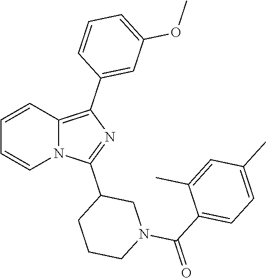 [SMILES: COc1cccc(-c2nc(C3CCCN(C(=O)c4ccc(C)cc4C)C3)n3ccccc23)c1] |
| 12-9 | 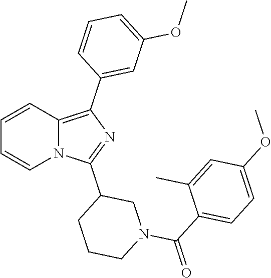 [SMILES: COc1cccc(-c2nc(C3CCCN(C(=O)c4ccc(OC)cc4C)C3)n3ccccc23)c1] |
| 12-10 | 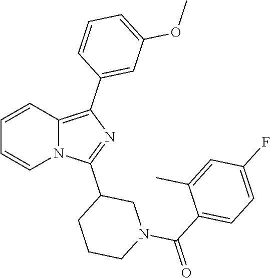 [SMILES: COc1cccc(-c2nc(C3CCCN(C(=O)c4ccc(F)cc4C)C3)n3ccccc23)c1] |
| 12-11 | 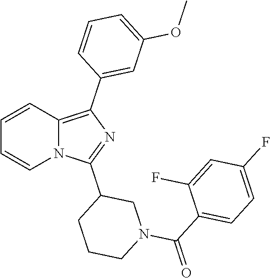 [SMILES: COc1cccc(-c2nc(C3CCCN(C(=O)c4ccc(F)cc4F)C3)n3ccccc23)c1] |
| 12-12 | 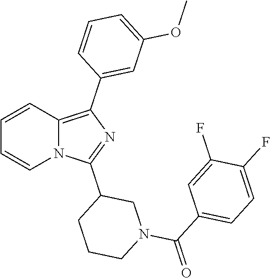 [SMILES: COc1cccc(-c2nc(C3CCCN(C(=O)c4ccc(F)c(F)c4)C3)n3ccccc23)c1] |
| 12-13 | 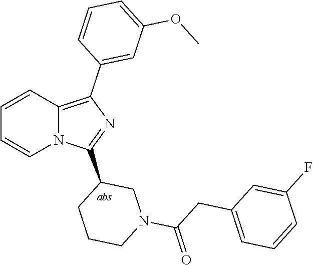 [SMILES: COc1cccc(-c2nc([C@@H]3CCCN(C(=O)Cc4cccc(F)c4)C3)n3ccccc23)c1] |
| 12-14 | 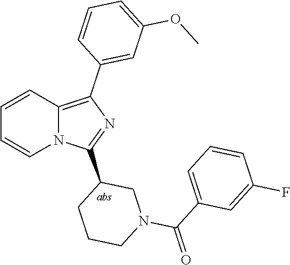 [SMILES: COc1cccc(-c2nc([C@@H]3CCCN(C(=O)c4cccc(F)c4)C3)n3ccccc23)c1] |
| 12-15 | 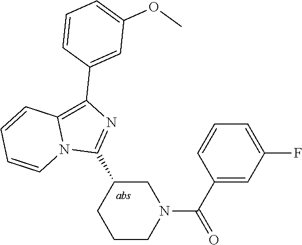 [SMILES: COc1cccc(-c2nc([C@H]3CCCN(C(=O)c4cccc(F)c4)C3)n3ccccc23)c1] |
| 12-16 | 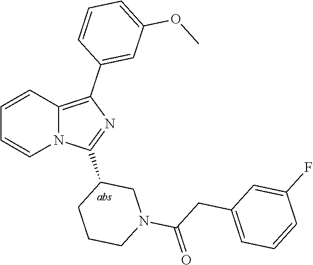 [SMILES: COc1cccc(-c2nc([C@H]3CCCN(C(=O)Cc4cccc(F)c4)C3)n3ccccc23)c1] |
| 14-1 | 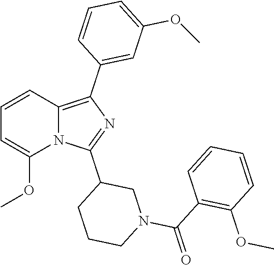 [SMILES: COc1cccc(-c2nc(C3CCCN(C(=O)c4ccccc4OC)C3)n3c(OC)cccc23)c1] |
| 14-2 | 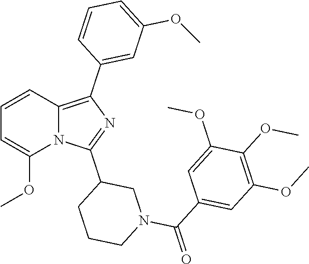 [SMILES: COc1cccc(-c2nc(C3CCCN(C(=O)c4cc(OC)c(OC)c(OC)c4)C3)n3c(OC)cccc23)c1] |
| 14-3 | 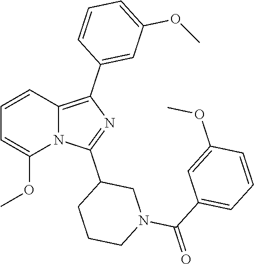 [SMILES: COc1cccc(C(=O)N2CCCC(c3nc(-c4cccc(OC)c4)c4cccc(OC)n34)C2)c1] |
| 14-4 | 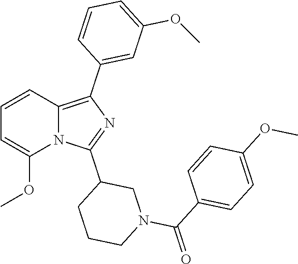 [SMILES: COc1ccc(C(=O)N2CCCC(c3nc(-c4cccc(OC)c4)c4cccc(OC)n34)C2)cc1] |
| 14-5 | 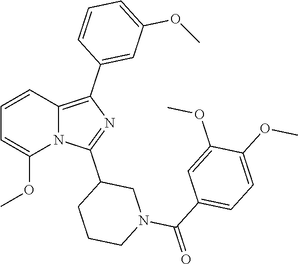 [SMILES: COc1cccc(-c2nc(C3CCCN(C(=O)c4ccc(OC)c(OC)c4)C3)n3c(OC)cccc23)c1] |
| 15-1 | 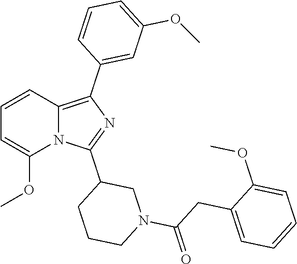 [SMILES: COc1cccc(-c2nc(C3CCCN(C(=O)Cc4ccccc4OC)C3)n3c(OC)cccc23)c1] |
| 16-1 | 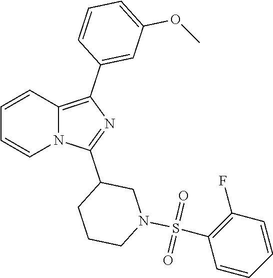 [SMILES: COc1cccc(-c2nc(C3CCCN(S(=O)(=O)c4ccccc4F)C3)n3ccccc23)c1] |
| 16-2 | 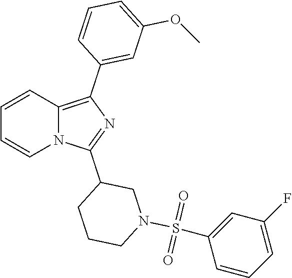 [SMILES: COc1cccc(-c2nc(C3CCCN(S(=O)(=O)c4cccc(F)c4)C3)n3ccccc23)c1] |
| 16-3 | 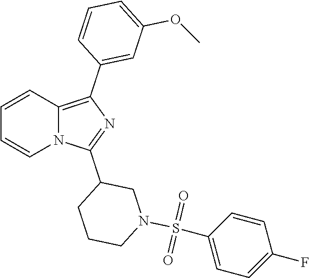 [SMILES: COc1cccc(-c2nc(C3CCCN(S(=O)(=O)c4ccc(F)cc4)C3)n3ccccc23)c1] |
| 16-4 | 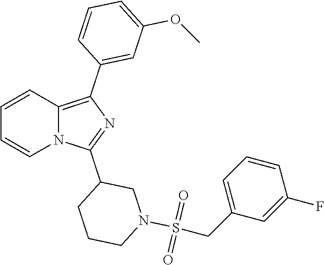 [SMILES: COc1cccc(-c2nc(C3CCCN(S(=O)(=O)Cc4cccc(F)c4)C3)n3ccccc23)c1] |
| 16-5 | 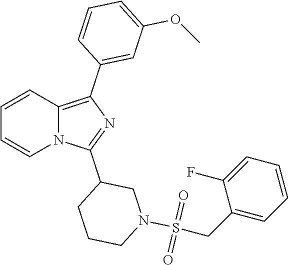 [SMILES: COc1cccc(-c2nc(C3CCCN(S(=O)(=O)Cc4ccccc4F)C3)n3ccccc23)c1] |
| 16-6 | 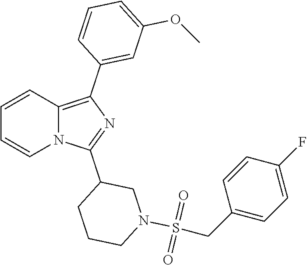 [SMILES: COc1cccc(-c2nc(C3CCCN(S(=O)(=O)Cc4ccc(F)cc4)C3)n3ccccc23)c1] |
| 16-7 | 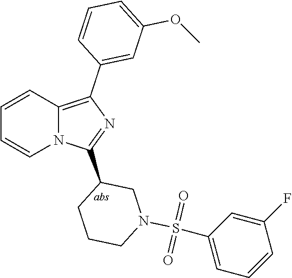 [SMILES: COc1cccc(-c2nc([C@@H]3CCCN(S(=O)(=O)c4cccc(F)c4)C3)n3ccccc23)c1] |
| 16-8 | 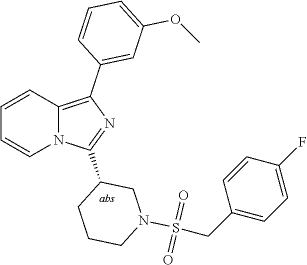 [SMILES: COc1cccc(-c2nc([C@H]3CCCN(S(=O)(=O)Cc4ccc(F)cc4)C3)n3ccccc23)c1] |
| 16-9 | 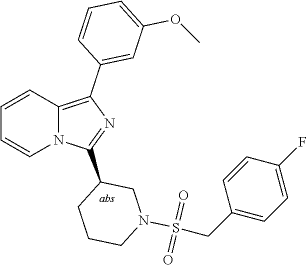 [SMILES: COc1cccc(-c2nc([C@@H]3CCCN(S(=O)(=O)Cc4ccc(F)cc4)C3)n3ccccc23)c1] |
| 16-10 | 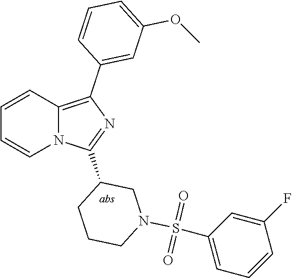 [SMILES: COc1cccc(-c2nc([C@H]3CCCN(S(=O)(=O)c4cccc(F)c4)C3)n3ccccc23)c1] |

In some embodiments, compounds having activity as modulators of the Mas-related G-protein coupled receptor D are provided, the compounds having the following structure (X):

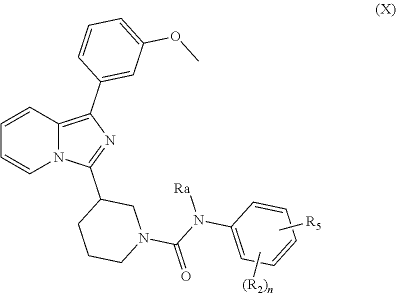

or a pharmaceutically acceptable salt, isomer, hydrate, solvate, or isotope thereof, wherein:
R₂ is hydroxyl, halo, cyano, alkyl, haloalkyl, hydroxylalkyl, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₅ is alkyl;
Ra is H or alkyl; and
n is 1, 2, or 3.

In some embodiments, compounds of structure (X) are provided wherein R₂ is hydroxyl, halo, cyano, alkyl, haloalkyl, hydroxylalkyl, haloalkoxy, aminyl, aminylalkyl, cyanoalkyl, alkoxyalkyl, alkylcarbonyl, aminylcarbonyl, alkylsulfonyl, or alkylsufonylalkyl. In some embodiments, compounds of structure (X) are provided wherein R₂ is hydroxyl, halo, cyano, alkyl, haloalkyl, hydroxylalkyl, haloalkoxy, aminyl, aminylalkyl, cyanoalkyl, alkoxyalkyl, alkylcarbonyl, aminylcarbonyl, or alkylsulfonyl. In other embodiments, compounds of structure (X) are provided wherein R₂ is aryl, heterocyclyl, or alkylheteroaryl. In certain embodiments, compounds of structure (X) are provided wherein R₂ is selected from:

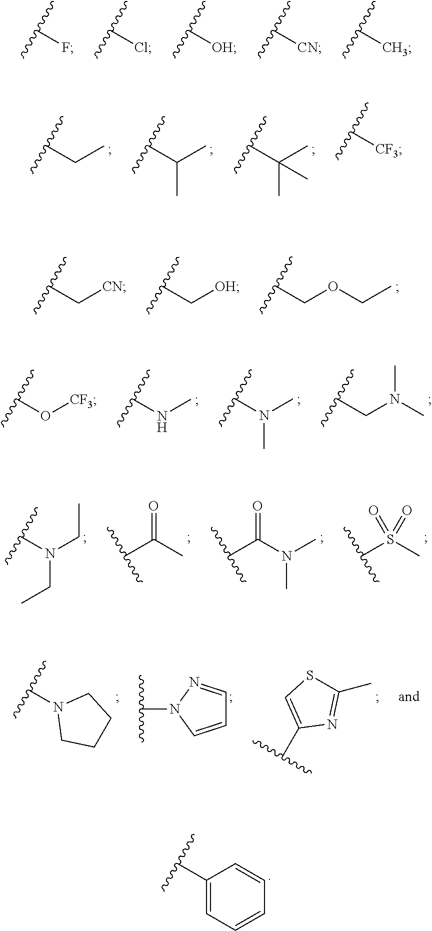

In specific embodiments, compounds of structure (X) are provided wherein R₂ is selected from halo and alkyl. In specific embodiments, compounds of structure (X) are provided wherein R₂ is selected from:

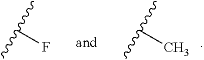

In some embodiments, compounds of structure (X) are provided wherein Ra is H. In other embodiments, compounds of structure (X) are provided wherein Ra is alkyl.

In other embodiments, compounds of structure (X) are provided wherein n is 1. In additional embodiments, compounds of structure (X) are provided wherein n is 2. In further embodiments, compounds of structure (X) are provided wherein n is 3.

Representative compounds of structure (X) include any one of the compounds listed in Table X below, as well as a pharmaceutically acceptable isomer, racemate, hydrate, solvate, isotope, or salt thereof.

TABLE X
Compounds of Structure X

| Cpd. No. | Structure |
|---|---|
| 1-10 | 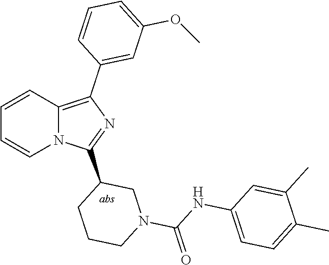 [SMILES: COc1cccc(-c2nc([C@@H]3CCCN(C(=O)Nc4ccc(C)c(C)c4)C3)n3ccccc23)c1] |
| 1-11 | 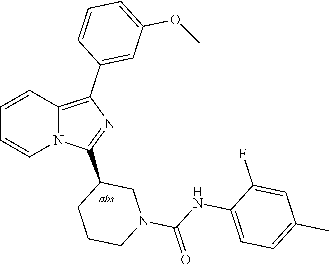 [SMILES: COc1cccc(-c2nc([C@@H]3CCCN(C(=O)Nc4ccc(C)cc4F)C3)n3ccccc23)c1] |
| 3-23 | 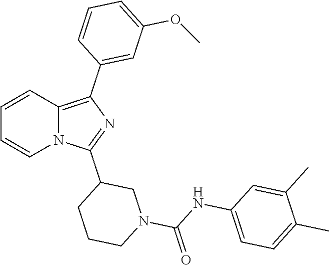 [SMILES: COc1cccc(-c2nc(C3CCCN(C(=O)Nc4ccc(C)c(C)c4)C3)n3ccccc23)c1] |
| 5-3 | 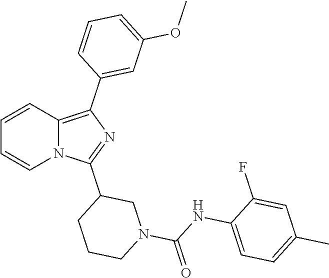 [SMILES: COc1cccc(-c2nc(C3CCCN(C(=O)Nc4ccc(C)cc4F)C3)n3ccccc23)c1] |
| 5-5 | 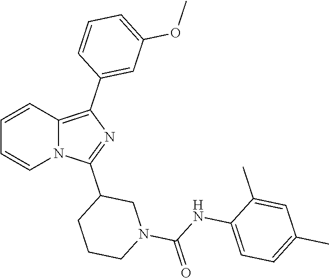 [SMILES: COc1cccc(-c2nc(C3CCCN(C(=O)Nc4ccc(C)cc4C)C3)n3ccccc23)c1] |
| 5-25 | 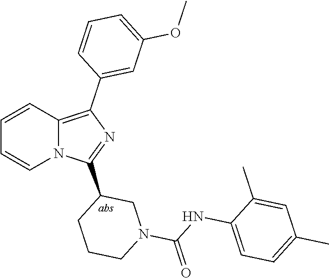 [SMILES: COc1cccc(-c2nc([C@@H]3CCCN(C(=O)Nc4ccc(C)cc4C)C3)n3ccccc23)c1] |

In some embodiments, compounds having activity as modulators of the Mas-related G-protein coupled receptor D are provided, the compounds having the following structure (XI):

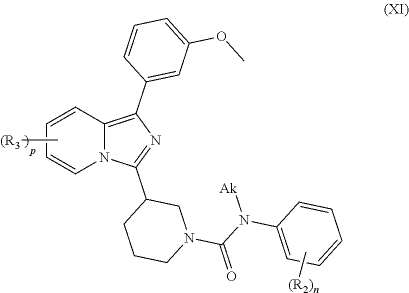

or a pharmaceutically acceptable salt, isomer, hydrate, solvate, or isotope thereof, wherein:
Ak is alkyl;
R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
n is 0, 1, 2, or 3; and
p is 0, 1, 2, or 3.

In some embodiments, compounds of structure (XI) are provided wherein R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, or alkylsufonylalkyl. In some embodiments, compounds of structure (XI) are provided wherein R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, or alkylsulfonyl. In other embodiments, compounds of structure (XI) are provided wherein R₂ is aryl, heterocyclyl, or alkylheteroaryl. In certain embodiments, compounds of structure (XI) are provided wherein R₂ is selected from:

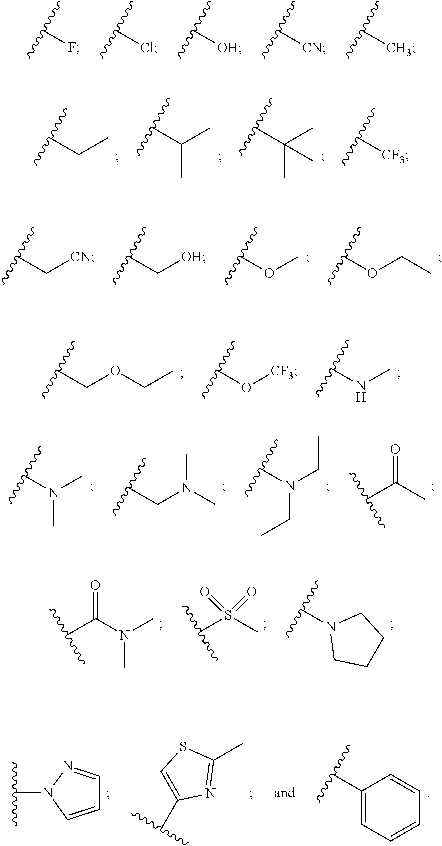

In specific embodiments, compounds of structure (XI) are provided wherein R₂ is halo. In specific embodiments, compounds of structure (XI) are provided wherein R₂ is

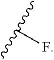 [SMILES: *F]

In some embodiments, compounds of structure (XI) are provided wherein R₃ is alkyl. In other embodiments, compounds of structure (XI) are provided wherein R₃ is alkoxy.

In some embodiments, compounds of structure (XI) are provided wherein n is 0. In other embodiments, compounds of structure (XI) are provided wherein n is 1. In additional embodiments, compounds of structure (XI) are provided wherein n is 2. In further embodiments, compounds of structure (XI) are provided wherein n is 3.

In some embodiments, compounds of structure (XI) are provided wherein p is 0. In other embodiments, compounds of structure (XI) are provided wherein p is 1. In additional embodiments, compounds of structure (XI) are provided wherein p is 2. In further embodiments, compounds of structure (XI) are provided wherein p is 3.

Representative compounds of structure (XI) include any one of the compounds listed in Table XI below, as well as a pharmaceutically acceptable isomer, racemate, hydrate, solvate, isotope, or salt thereof.

TABLE XI
Compounds of Structure XI

| Cpd. No. | Structure |
|---|---|
| 1-3 | 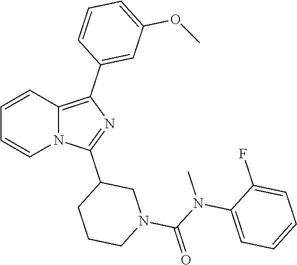 [SMILES: COc1cccc(-c2nc(C3CCCN(C(=O)N(C)c4ccccc4F)C3)n3ccccc23)c1] |
| 1-4 | 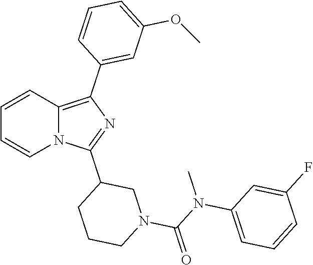 [SMILES: COc1cccc(-c2nc(C3CCCN(C(=O)N(C)c4cccc(F)c4)C3)n3ccccc23)c1] |
| 5-1 | 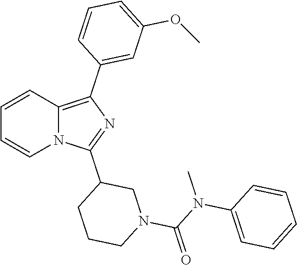 [SMILES: COc1cccc(-c2nc(C3CCCN(C(=O)N(C)c4ccccc4)C3)n3ccccc23)c1] |
| 5-2 | 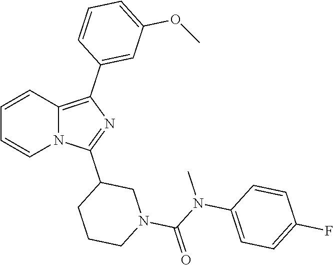 [SMILES: COc1cccc(-c2nc(C3CCCN(C(=O)N(C)c4ccc(F)cc4)C3)n3ccccc23)c1] |

In some embodiments, compounds having activity as modulators of the Mas-related G-protein coupled receptor D are provided, the compounds having the following structure (XII):

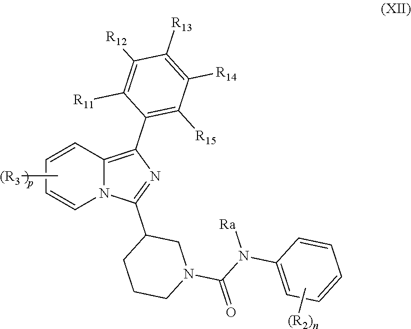

or a pharmaceutically acceptable salt, isomer, hydrate, solvate, or isotope thereof, wherein:
either (1) R₁₃ is methoxy and R₁₁, R₁₂, R₁₄, and R₁₅ are each individually H,
(2) R₁₁ is methoxy and R₁₂, R₁₃, R₁₄, and R₁₅ are each individually H,
(3) R₁₅ is methoxy and R₁₁, R₁₂, R₁₃, and R₁₄ are each individually H, or
(4) R₁₂ and R₁₄ are both methoxy and R₁₁, R₁₃, and R₁₅ are each individually H; and
R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
Ra is H or alkyl;
n is 0, 1, 2, or 3; and
p is 0, 1, 2, or 3.

In some embodiments, compounds of structure (XII) are provided wherein R₁₃ is methoxy and R₁₁, R₁₂, R₁₄, and R₁₅ are each individually H.

In some embodiments, compounds of structure (XII) are provided wherein R₁₁ is methoxy and R₁₂, R₁₃, R₁₄, and R₁₅ are each individually H.

In some embodiments, compounds of structure (XII) are provided wherein R₁₅ is methoxy and R₁₁, R₁₂, R₁₃, and R₁₄ are each individually H.

In some embodiments, compounds of structure (XII) are provided wherein R₁₂ and R₁₄ are both methoxy and R₁₁, R₁₃, and R₁₅ are each individually H.

In some embodiments, compounds of structure (XII) are provided wherein R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, or alkylsufonylalkyl. In some embodiments, compounds of structure (XII) are provided wherein R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, or alkylsulfonyl. In other embodiments, compounds of structure (XII) are provided wherein R₂ is aryl, heterocyclyl, or alkylheteroaryl. In certain embodiments, compounds of structure (XII) are provided wherein R₂ is selected from:

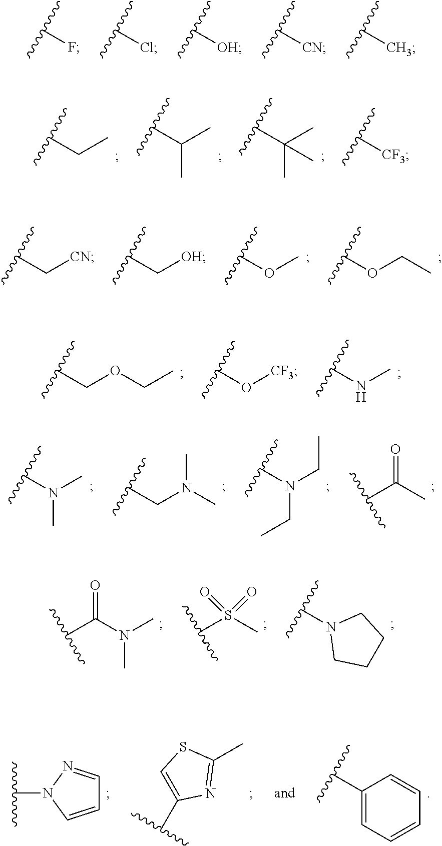

In specific embodiments, compounds of structure (XII) are provided wherein R₂ is selected from halo and aminyl. In specific embodiments, compounds of structure (XII) are provided wherein R₂ is selected from:

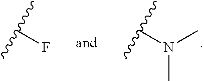

In some embodiments, compounds of structure (XII) are provided wherein R₃ is alkyl. In other embodiments, compounds of structure (XII) are provided wherein R₃ is alkoxy.

In some embodiments, compounds of structure (XII) are provided wherein Ra is H. In other embodiments, compounds of structure (XII) are provided wherein Ra is alkyl.

In some embodiments, compounds of structure (XII) are provided wherein n is 0. In other embodiments, compounds of structure (XII) are provided wherein n is 1. In additional embodiments, compounds of structure (XII) are provided wherein n is 2. In further embodiments, compounds of structure (XII) are provided wherein n is 3.

In some embodiments, compounds of structure (XII) are provided wherein p is 0. In other embodiments, compounds of structure (XII) are provided wherein p is 1. In additional embodiments, compounds of structure (XII) are provided wherein p is 2. In further embodiments, compounds of structure (XII) are provided wherein p is 3.

Representative compounds of structure (XII) include any one of the compounds listed in Table XII below, as well as a pharmaceutically acceptable isomer, racemate, hydrate, solvate, isotope, or salt thereof.

TABLE XII
Compounds of Structure XII

| Cpd. No. | Structure |
|---|---|
| 10-17 | 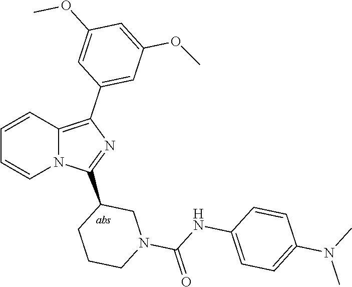 [SMILES: COc1cc(OC)cc(-c2nc([C@@H]3CCCN(C(=O)Nc4ccc(N(C)C)cc4)C3)n3ccccc23)c1] |
| 10-31 | 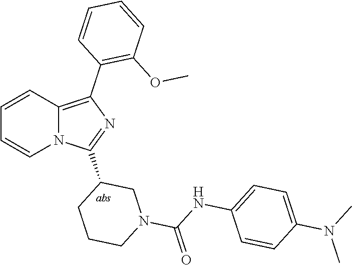 [SMILES: COc1ccccc1-c1nc([C@H]2CCCN(C(=O)Nc3ccc(N(C)C)cc3)C2)n2ccccc12] |
| 10-37 | 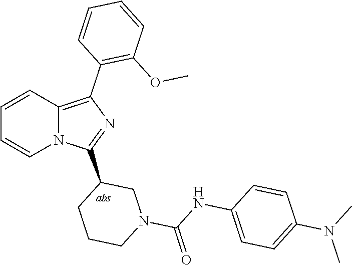 [SMILES: COc1ccccc1-c1nc([C@@H]2CCCN(C(=O)Nc3ccc(N(C)C)cc3)C2)n2ccccc12] |
| 10-43 | 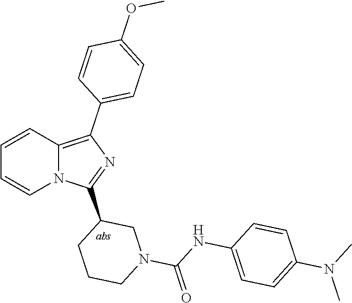 [SMILES: COc1ccc(-c2nc([C@@H]3CCCN(C(=O)Nc4ccc(N(C)C)cc4)C3)n3ccccc23)cc1] |
| 10-48 | 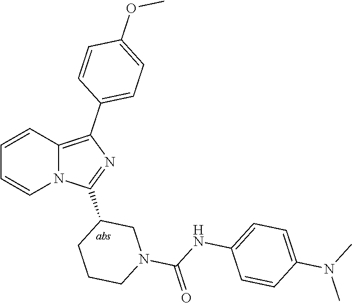 [SMILES: COc1ccc(-c2nc([C@H]3CCCN(C(=O)Nc4ccc(N(C)C)cc4)C3)n3ccccc23)cc1] |
| 11-3 | 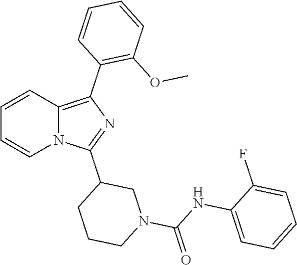 [SMILES: COc1ccccc1-c1nc(C2CCCN(C(=O)Nc3ccccc3F)C2)n2ccccc12] |
| 11-9 | 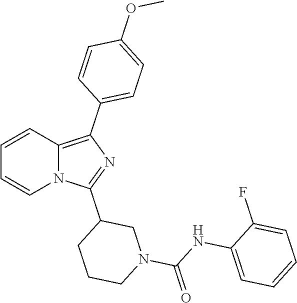 [SMILES: COc1ccc(-c2nc(C3CCCN(C(=O)Nc4ccccc4F)C3)n3ccccc23)cc1] |

In some embodiments, compounds having activity as modulators of the Mas-related G-protein coupled receptor D are provided, the compounds having the following structure (XIII):

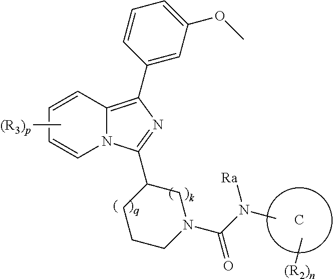

or a pharmaceutically acceptable salt, isomer, hydrate, solvate, or isotope thereof, wherein:
either (1) k is 0 and q is 1, or (2) k is 1 and q is 0;
C is phenyl, or C is methyl and n is 0;
R₂ is hydroxyl, halo, cyano, haloalkyl, hydroxylalkyl, alkoxy, haloalkoxy, aminyl, cyanoalkyl, alkoxyalkyl, alkylcarbonyl, aminylcarbonyl, alkylsulfonyl, aminylalkyl, alkylsufonylalkyl, aryl, heterocyclyl, or alkylheteroaryl;
R₃ is alkyl or alkoxy;
Ra is H or alkyl;
n is 0, 1, 2, or 3; and
p is 0, 1, 2, or 3.

In some embodiments, compounds of structure (XIII) are provided wherein k is 0 and q is 1.

In some embodiments, compounds of structure (XIII) are provided wherein k is 1 and q is 0.

In some embodiments, compounds of structure (XIII) are provided wherein C is phenyl. In other embodiments, compounds of structure (XIII) are provided wherein C is methyl and n is 0.

In some embodiments, compounds of structure (XIII) are provided wherein R₂ is hydroxyl, halo, cyano, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, or alkylsufonylalkyl. In some embodiments, compounds of structure (XIII) are provided wherein R₂ is hydroxyl, halo, cyano, haloalkyl, hydroxylalkyl, alkoxy, haloalkoxy, aminyl, aminylalkyl, cyanoalkyl, alkoxyalkyl, alkylcarbonyl, aminylcarbonyl, or alkylsulfonyl. In other embodiments, compounds of structure (XIII) are provided wherein R₂ is aryl, heterocyclyl, or alkylheteroaryl. In certain embodiments, compounds of structure (XIII) are provided wherein R₂ is selected from:

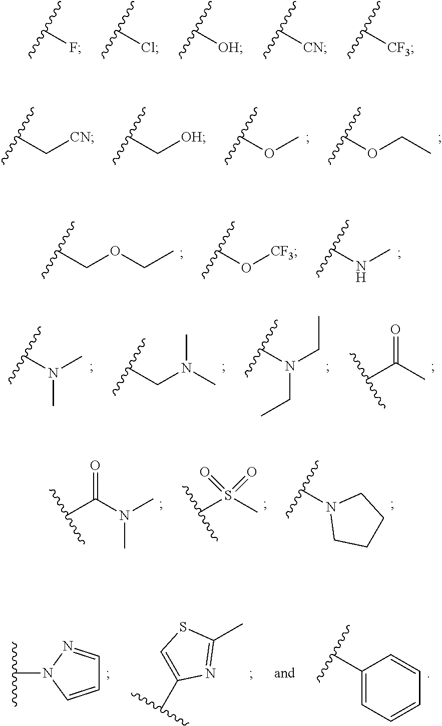

In specific embodiments, compounds of structure (XIII) are provided wherein R₂ is selected from halo and aminyl. In specific embodiments, compounds of structure (XIII) are provided wherein R₂ is selected from:

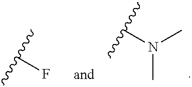

In some embodiments, compounds of structure (XIII) are provided wherein R₃ is alkyl. In other embodiments, compounds of structure (XIII) are provided wherein R₃ is alkoxy.

In some embodiments, compounds of structure (XIII) are provided wherein Ra is H. In other embodiments, compounds of structure (XIII) are provided wherein Ra is alkyl.

In some embodiments, compounds of structure (XIII) are provided wherein n is 0. In other embodiments, compounds of structure (XIII) are provided wherein n is 1. In additional embodiments, compounds of structure (XIII) are provided wherein n is 2. In further embodiments, compounds of structure (XIII) are provided wherein n is 3.

In some embodiments, compounds of structure (XIII) are provided wherein p is 0. In other embodiments, compounds of structure (XIII) are provided wherein p is 1. In additional embodiments, compounds of structure (XIII) are provided wherein p is 2. In further embodiments, compounds of structure (XIII) are provided wherein p is 3.

Representative compounds of structure (XIII) include any one of the compounds listed in Table XIII below, as well as a pharmaceutically acceptable isomer, racemate, hydrate, solvate, isotope, or salt thereof.

TABLE XIII
Compounds of Structure XIII

| Cpd. No. | Structure |
|---|---|
| 21-1 | 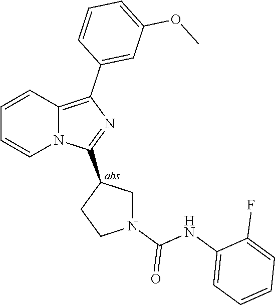 [SMILES: COc1cccc(-c2nc([C@@H]3CCN(C(=O)Nc4ccccc4F)C3)n3ccccc23)c1] |
| 21-2 | 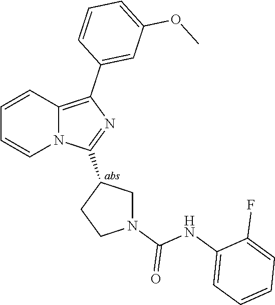 [SMILES: COc1cccc(-c2nc([C@H]3CCN(C(=O)Nc4ccccc4F)C3)n3ccccc23)c1] |
| 21-3 | 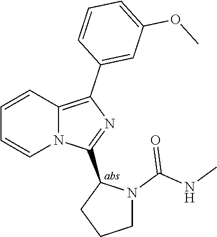 [SMILES: CNC(=O)N1CCC[C@H]1c1nc(-c2cccc(OC)c2)c2ccccn12] |
| 21-4 | 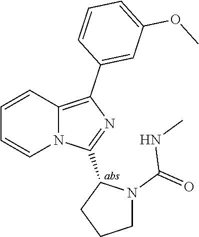 [SMILES: CNC(=O)N1CCC[C@@H]1c1nc(-c2cccc(OC)c2)c2ccccn12] |
| 21-5 | 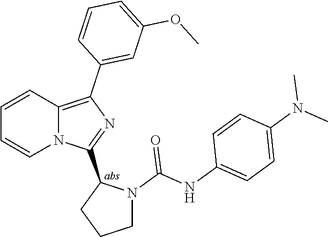 [SMILES: COc1cccc(-c2nc([C@@H]3CCCN3C(=O)Nc3ccc(N(C)C)cc3)n3ccccc23)c1] |
| 21-6 | 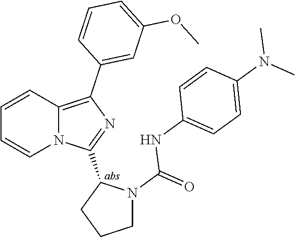 [SMILES: COc1cccc(-c2nc([C@H]3CCCN3C(=O)Nc3ccc(N(C)C)cc3)n3ccccc23)c1] |
| 22-1 | 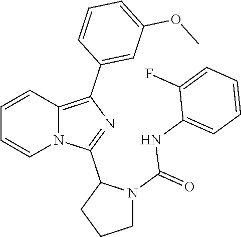 [SMILES: COc1cccc(-c2nc(C3CCCN3C(=O)Nc3ccccc3F)n3ccccc23)c1] |

In some embodiments, compounds having activity as modulators of the Mas-related G-protein coupled receptor D are provided, the compounds having the following structure (XIV):

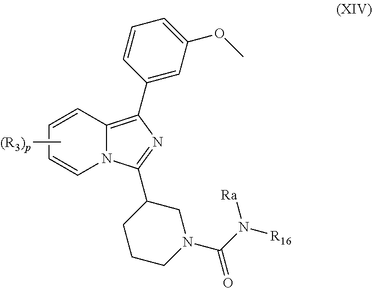

or a pharmaceutically acceptable salt, isomer, hydrate, solvate, or isotope thereof, wherein:
R₁₆ is methyl, alkoxyalkyl, alkylsufonylalkyl, C₃-C₈ cycloalkyl, C₃-C₅ cycloalkylC₁-C₆alkyl, alkylC₃-C₅cycloalkyl, benzyl,

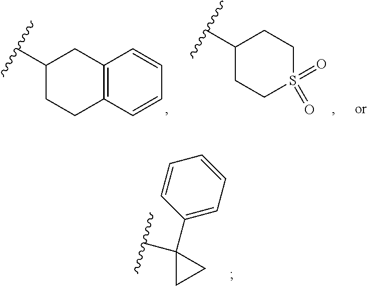 [SMILES: *C1CCc2ccccc2C1 ; *C1CCS(=O)(=O)CC1 ; *C1(c2ccccc2)CC1]

R₃ is alkyl or alkoxy; and
Ra is H or alkyl;
p is 0, 1, 2, or 3.

In some embodiments, compounds of structure (XIV) are provided wherein R₁₆ is methyl. In other embodiments, compounds of structure (XIV) are provided wherein R₁₆ is alkoxyalkyl. In additional embodiments, compounds of structure (XIV) are provided wherein R₁₆ is

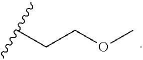 [SMILES: *CCOC ; "."]

In further embodiments, compounds of structure (XIV) are provided wherein R₁₆ is alkylsufonylalkyl. In yet other embodiments, compounds of structure (XIV) are provided wherein R₁₆ is

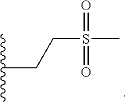 [SMILES: *CCS(C)(=O)=O]

In some embodiments, compounds of structure (XIV) are provided wherein R₁₆ is C₃-C₈ cycloalkyl. In certain embodiments, compounds of structure (XIV) are provided wherein R₁₆ is

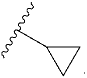 [SMILES: *C1CC1]

In other embodiments, compounds of structure (XIV) are provided wherein R₁₆ is C₃-C₈ cycloalkylC₁-C₆alkyl. In specific embodiments, compounds of structure (XIV) are provided wherein R₁₆ is

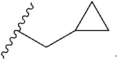 [SMILES: *CC1CC1]

In yet other embodiments, compounds of structure (XIV) are provided wherein R₁₆ is alkylC₃-C₅cycloalkyl. In additional embodiments, compounds of structure (XIV) are provided wherein R₁₆ is

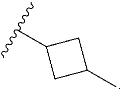 [SMILES: *C1CC(C)C1]

In further embodiments, compounds of structure (XIV) are provided wherein R₁₆ is benzyl. In yet other embodiments, compounds of structure (XIV) are provided wherein R₁₆ is

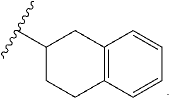 [SMILES: *C1CCc2ccccc2C1]

In some embodiments, compounds of structure (XIV) are provided wherein R₁₆ is

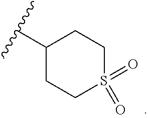 [SMILES: *C1CCS(=O)(=O)CC1]

In specific embodiments, compounds of structure (XIV) are provided wherein R₁₆ is or

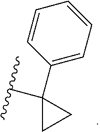 [SMILES: *C1(c2ccccc2)CC1]

In some embodiments, compounds of structure (XIV) are provided wherein R₃ is alkyl. In other embodiments, compounds of structure (XIV) are provided wherein R₃ is alkoxy.

In some embodiments, compounds of structure (XIV) are provided wherein Ra is H. In other embodiments, compounds of structure (XIV) are provided wherein Ra is alkyl.

In some embodiments, compounds of structure (XIV) are provided wherein p is 0. In other embodiments, compounds of structure (XIV) are provided wherein p is 1. In additional embodiments, compounds of structure (XIV) are provided wherein p is 2. In further embodiments, compounds of structure (XIV) are provided wherein p is 3.

Representative compounds of structure (XIV) include any one of the compounds listed in Table XIV below, as well as a pharmaceutically acceptable isomer, racemate, hydrate, solvate, isotope, or salt thereof.

TABLE XIV
Compounds of Structure XIV

| Cpd. No. | Structure |
|---|---|
| 2-1 | 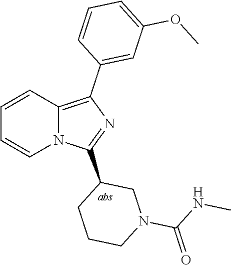 [SMILES: CNC(=O)N1CCC[C@@H](c2nc(-c3cccc(OC)c3)c3ccccn23)C1] |
| 2-2 | 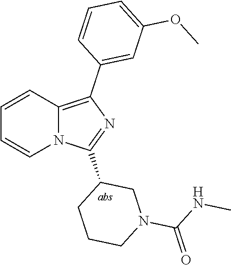 [SMILES: CNC(=O)N1CCC[C@H](c2nc(-c3cccc(OC)c3)c3ccccn23)C1] |
| 3-11 | 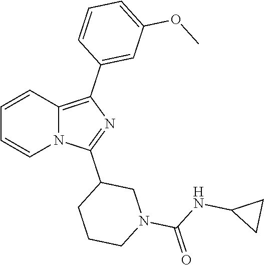 [SMILES: COc1cccc(-c2nc(C3CCCN(C(=O)NC4CC4)C3)n3ccccc23)c1] |
| 3-13 | 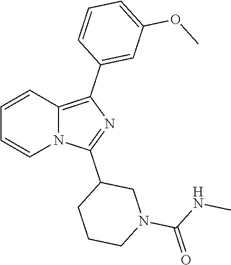 [SMILES: CNC(=O)N1CCCC(c2nc(-c3cccc(OC)c3)c3ccccn23)C1] |
| 3-14 | 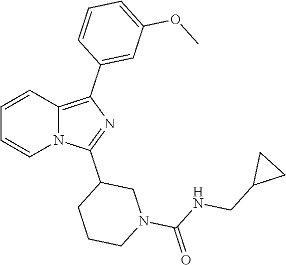 [SMILES: COc1cccc(-c2nc(C3CCCN(C(=O)NCC4CC4)C3)n3ccccc23)c1] |
| 5-38 | 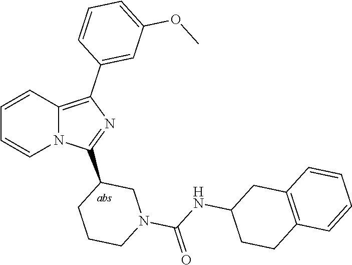 [SMILES: COc1cccc(-c2nc([C@@H]3CCCN(C(=O)NC4CCc5ccccc5C4)C3)n3ccccc23)c1] |
| 5-41 | 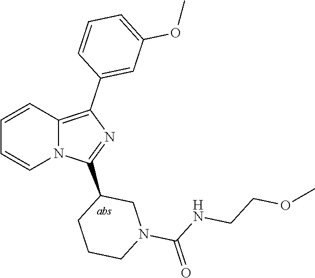 [SMILES: COCCNC(=O)N1CCC[C@@H](c2nc(-c3cccc(OC)c3)c3ccccn23)C1] |
| 5-48 | 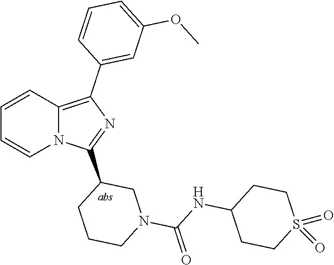 [SMILES: COc1cccc(-c2nc([C@@H]3CCCN(C(=O)NC4CCS(=O)(=O)CC4)C3)n3ccccc23)c1] |
| 5-52 | 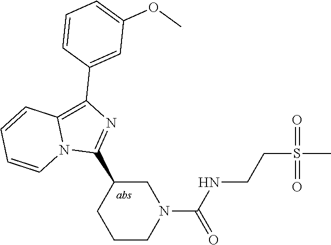 [SMILES: COc1cccc(-c2nc([C@@H]3CCCN(C(=O)NCCS(C)(=O)=O)C3)n3ccccc23)c1] |
| 5-55 | 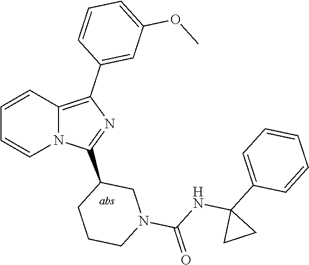 [SMILES: COc1cccc(-c2nc([C@@H]3CCCN(C(=O)NC4(c5ccccc5)CC4)C3)n3ccccc23)c1] |
| 5-60 | 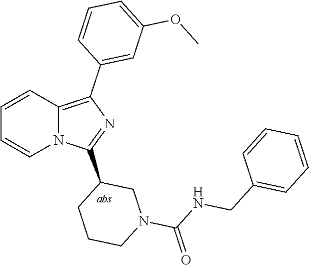 [SMILES: COc1cccc(-c2nc([C@@H]3CCCN(C(=O)NCc4ccccc4)C3)n3ccccc23)c1] |
| 5-71 | 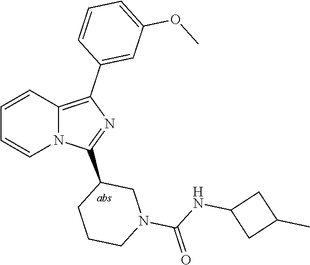 [SMILES: COc1cccc(-c2nc([C@@H]3CCCN(C(=O)NC4CC(C)C4)C3)n3ccccc23)c1] |

In some embodiments, provided is a method of treating a MRGPRD dependent condition by administering to a subject in need thereof an effective amount of a compound having the following structure (XV):

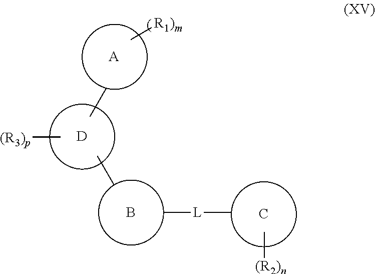

or a pharmaceutically acceptable salt, isomer, hydrate, solvate, or isotope thereof, wherein:
A is carbocyclyl, heterocyclyl, alkoxyalkyl, or alkoxyalkenyl;
B is phenyl, thiophenyl, piperidinyl, pyrrolidinyl, or azetidinyl;
C is carbocyclyl or heterocyclyl, or C is optionally absent when L is —C(O)—NRa- and R₂ is alkyl, aminylalkyl, alkoxyalkyl, or alkylsufonylalkyl;
D is

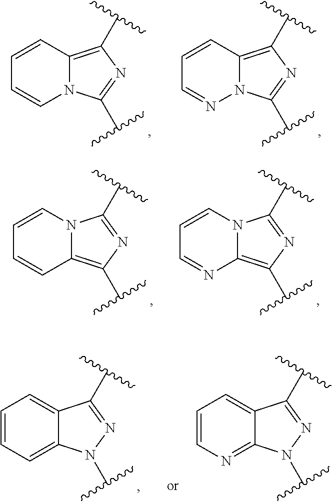 [SMILES: *c1nc(*)n2ccccc12 ; *c1nc(*)n2ncccc12 ; *c1nc(*)n2ccccc12 ; *c1nc(*)n2cccnc12 ; *c1nn(*)c2ccccc12 ; *c1nn(*)c2ncccc12]

L is a linker having the structure —C(O)—, —C(O)-alkyl-, —C(O)—NRa-, —C(O)—NRa-alkyl-, —S(O)₂—, or —S(O)₂-alkyl-;
R₁ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, alkoxycarbonyl, aminylcarbonyl, alkylsulfonyl, cycloalkylalkyl, aryl, aralkyl, or arylsulfonyl;
R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
Ra is H or alkyl;
m is 0, 1, or 2;
n is 0, 1, 2, or 3; and
p is 0, 1, 2, or 3.

In some embodiments, provided is a method of treating a MRGPRD dependent condition by administering to a subject in need thereof an effective amount of a compound having the following structure (XVa):

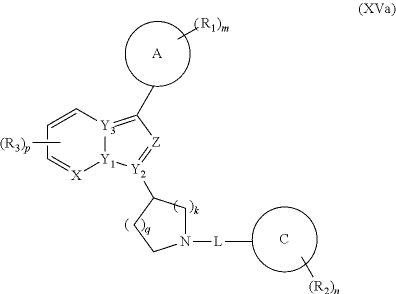

or a pharmaceutically acceptable salt, isomer, hydrate, solvate, or isotope thereof, wherein:
A is carbocyclyl, heterocyclyl, alkoxyalkyl, or alkoxyalkenyl;
C is carbocyclyl or heterocyclyl, or C is optionally absent when L is —C(O)—NRa- and R₂ is alkyl, aminylalkyl, alkoxyalkyl, or alkylsufonylalkyl;
X is CR₃, CH, or N;
Y₁, Y₂, and Y₃ are each independently C or N;
Z is CH or N;
L is a linker having the structure —C(O)—, —C(O)-alkyl-, —C(O)—NRa-, —C(O)—NRa-alkyl-, —S(O)₂—, or —S(O)₂-alkyl-;
R₁ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, alkoxycarbonyl, aminylcarbonyl, alkylsulfonyl, cycloalkylalkyl, aryl, aralkyl, or arylsulfonyl;
R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
Ra is H or alkyl;
m is 0, 1, or 2;
n is 0, 1, 2, or 3;
p is 0, 1, 2, or 3;
q is 0, 1, or 2; and
k is 0, 1, or 2.

In some embodiments, provided is a method of treating a MRGPRD dependent condition by administering to a subject in need thereof an effective amount of a compound of the structure (XVa), wherein Y₁ is N, Y₂ and Y₃ are each C, and Z is N. In other embodiments, Y₂ and Z are each N, and Y₁ and Y₃ are C. In additional embodiments, Y₃ is N, Y₁ and Y₂ are C, and Z is N.

In some embodiments, provided is a method of treating a MRGPRD dependent condition by administering to a subject in need thereof an effective amount of a compound of the structure (XVa), wherein q is 0 and k is 1. In other embodiments, q is 1 and k is 1. In additional embodiments, q is 1 and k is 2. In further embodiments, q is 2 and k is 0. In yet other embodiments, q is 2 and k is 1.

In some embodiments, provided is a method of treating a MRGPRD dependent condition by administering to a subject in need thereof an effective amount of a compound having the following structure (XVb):

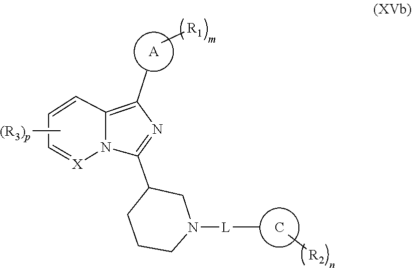

or a pharmaceutically acceptable salt, isomer, hydrate, solvate, or isotope thereof, wherein:
A is carbocyclyl, heterocyclyl, alkoxyalkyl, or alkoxyalkenyl;
C is carbocyclyl or heterocyclyl, or C is optionally absent when L is —C(O)—NRa- and R₂ is alkyl, aminylalkyl, alkoxyalkyl, or alkylsufonylalkyl;
X is CR₃, CH, or N;
L is a linker having the structure —C(O)—, —C(O)-alkyl-, —C(O)—NRa-, —C(O)—NRa-alkyl-, —S(O)₂—, or —S(O)₂-alkyl-;
R₁ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, alkoxycarbonyl, aminylcarbonyl, alkylsulfonyl, cycloalkylalkyl, aryl, aralkyl, or arylsulfonyl;
R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
Ra is H or alkyl;
m is 0, 1, or 2;
n is 0, 1, 2, or 3; and
p is 0, 1, 2, or 3.

In some embodiments, provided is a method of treating a MRGPRD dependent condition by administering to a subject in need thereof an effective amount of a compound of the structure (XV), (XVa), or (XVb), wherein A is carbocyclyl. In other embodiments, A is cycloalkyl. In other embodiments, A is aryl. In other embodiments, A is phenyl. In other embodiments, A is heterocyclyl. In other embodiments, A is saturated heterocyclyl. In other embodiments, A is heteroaryl. In certain embodiments, A is selected from:

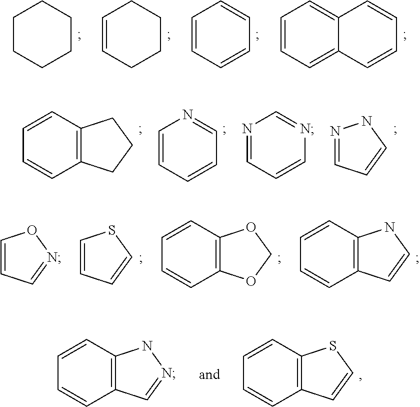

such that all valencies are satisfied. In additional embodiments, A is alkoxyalkyl. In specific embodiments, A is

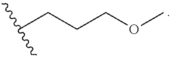 [SMILES: *CCCOC]

In further embodiments, A is alkoxyalkenyl. In specific embodiment, A is

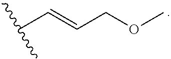 [SMILES: */C=C/COC]

In some embodiments, provided is a method of treating a MRGPRD dependent condition by administering to a subject in need thereof an effective amount of a compound of the structure (XV) wherein B is phenyl. In other embodiments, B is thiophenyl. In additional embodiments, B is piperidinyl. In further embodiments, B is pyrrolidinyl. In yet other embodiments, B is azetidinyl.

In some embodiments, provided is a method of treating a MRGPRD dependent condition by administering to a subject in need thereof an effective amount of a compound of the structure (XV), (XVa), or (XVb), wherein C is carbocyclyl. In other embodiments, C is cycloalkyl. In certain embodiments, C is cyclopropyl, cyclobutyl, cyclopentyl, or cyclohexyl. In additional embodiments, C is aryl. In specific embodiments, C is phenyl. In certain embodiments, C is selected from:

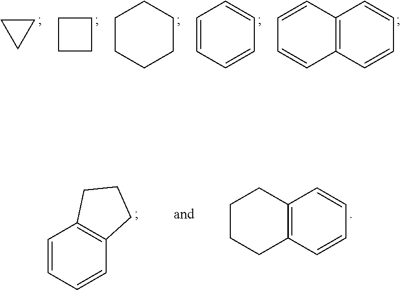

In further embodiments, C is heterocyclyl. In yet other embodiments, C is saturated heterocyclyl. In some embodiments, C is heteroaryl. In specific embodiments, C is selected from:

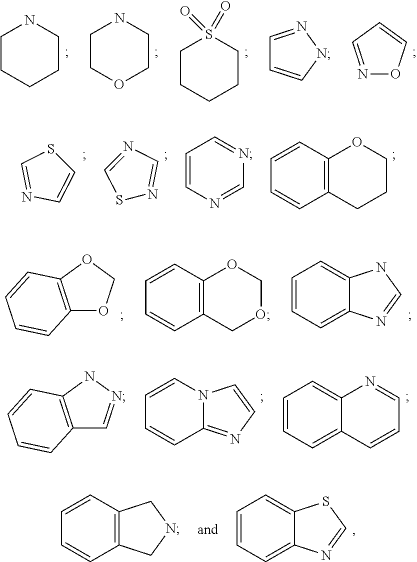

wherein all valencies are satisfied.

In some embodiments, provided is a method of treating a MRGPRD dependent condition by administering to a subject in need thereof an effective amount of a compound of the structure (XV), (XVa), or (XVb), wherein C is absent, L is —C(O)—NRa-, and R₂ is alkyl, aminylalkyl, alkoxyalkyl, or alkylsufonylalkyl. In certain embodiments, C is absent, L is —C(O)—NRa-, and R₂ is alkyl. In other embodiments, C is absent, L is —C(O)—NRa-, and R₂ is aminylalkyl. In additional embodiments, C is absent, L is —C(O)—NRa-, and R₂ is alkoxyalkyl. In further embodiments, C is absent, L is —C(O)—NRa-, and R₂ is alkylsufonylalkyl.

In some embodiments, provided is a method of treating a MRGPRD dependent condition by administering to a subject in need thereof an effective amount of a compound of the structure (XV), wherein D is

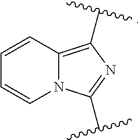 [SMILES: *c1nc(*)n2ccccc12]

In other embodiments, D is

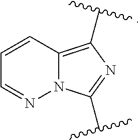 [SMILES: *c1nc(*)n2ncccc12]

In additional embodiments, D is

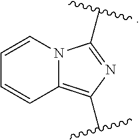 [SMILES: *c1nc(*)n2ccccc12]

In further embodiments, D is

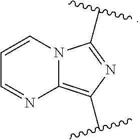 [SMILES: *c1nc(*)n2cccnc12]

In yet other embodiments, D is

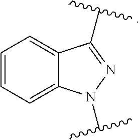 [SMILES: *c1nn(*)c2ccccc12]

In some embodiments, D is

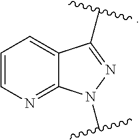 [SMILES: *c1nn(*)c2ncccc12]

In some embodiments, provided is a method of treating a MRGPRD dependent condition by administering to a subject in need thereof an effective amount of a compound of the structure (XVa) or (XVb), wherein X is CR₃. In other embodiments X is CH. In additional embodiments, X is N.

In some embodiments, provided is a method of treating a MRGPRD dependent condition by administering to a subject in need thereof an effective amount of a compound of the structure (XV), (XVa), or (XVb), wherein L is a linker having the structure —C(O)—. In other embodiments, L is a linker having the structure —C(O)-alkyl-. In additional embodiments, L is a linker having the structure —C(O)—NRa-. In further embodiments, L is a linker having the structure —C(O)—NRa-alkyl-. In yet other embodiments, L is a linker having the structure —S(O)₂—. In some embodiments, L is a linker having the structure —S(O)₂-alkyl-.

In some embodiments, provided is a method of treating a MRGPRD dependent condition by administering to a subject in need thereof an effective amount of a compound of the structure (XV), (XVa), or (XVb), wherein R₁ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, alkoxycarbonyl, aminylcarbonyl, or alkylsulfonyl. In other embodiments, R₁ is cycloalkylalkyl, aryl, aralkyl, or arylsulfonyl. In certain embodiments, R₁ is selected from:

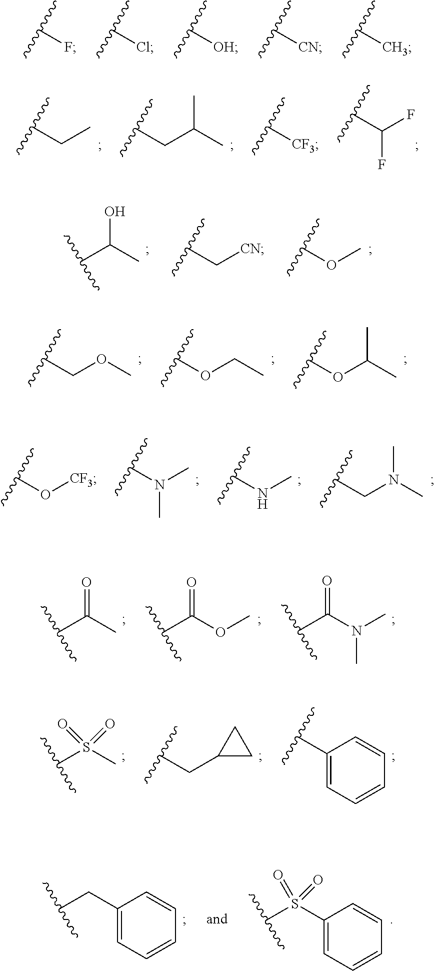

In some embodiments, provided is a method of treating a MRGPRD dependent condition by administering to a subject in need thereof an effective amount of a compound of the structure (XV), (XVa), or (XVb), wherein R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, or alkylsufonylalkyl. In some embodiments, provided is a method of treating a MRGPRD dependent condition by administering to a subject in need thereof an effective amount of a compound of the structure (XV), (XVa), or (XVb), wherein R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, or alkylsulfonyl. In other embodiments R₂ is aryl, heterocyclyl, or alkylheteroaryl. In certain embodiments, R₂ is selected from:

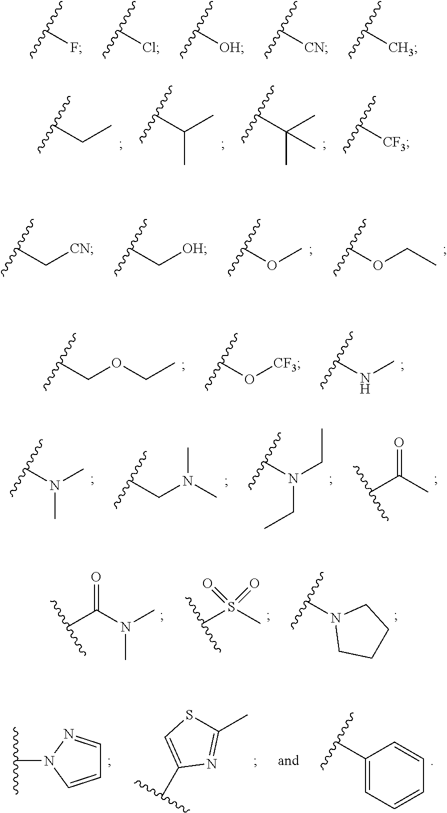

In some embodiments, provided is a method of treating a MRGPRD dependent condition by administering to a subject in need thereof an effective amount of a compound of the structure (XV), (XVa), or (XVb), wherein R₃ is alkyl. In other embodiments, R₃ is alkoxy.

In some embodiments, provided is a method of treating a MRGPRD dependent condition by administering to a subject in need thereof an effective amount of a compound of the structure (XV), (XVa), or (XVb), wherein Ra is H. In other embodiments, Ra is alkyl.

In some embodiments, provided is a method of treating a MRGPRD dependent condition by administering to a subject in need thereof an effective amount of a compound of the structure (XV), (XVa), or (XVb), wherein m is 0. In other embodiments, m is 1. In additional embodiments, m is 2.

In some embodiments, provided is a method of treating a MRGPRD dependent condition by administering to a subject in need thereof an effective amount of a compound of the structure (XV), (XVa), or (XVb), wherein n is 0. In other embodiments, n is 1. In additional embodiments, n is 2. In further embodiments, n is 3.

In some embodiments, provided is a method of treating a MRGPRD dependent condition by administering to a subject in need thereof an effective amount of a compound of the structure (XV), (XVa), or (XVb), wherein p is 0. In other embodiments, p is 1. In additional embodiments, p is 2. In further embodiments, p is 3.

Representative compounds of structures (XV), (XVa), or (XVb) are listed in Table XV below, and include pharmaceutically acceptable isomers, racemates, hydrates, solvates, isotopes, or salts thereof.

TABLE XV
Compounds of Structures (XV), (XVa), or (XVb)

| | Cpd. |
|---|---|
| Structure | No. |
| 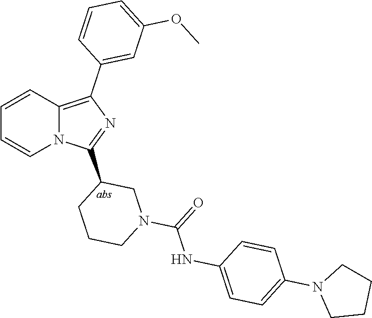 [SMILES: COc1cccc(-c2nc([C@@H]3CCCN(C(=O)Nc4ccc(N5CCCC5)cc4)C3)n3ccccc23)c1] | 1-1 |
| 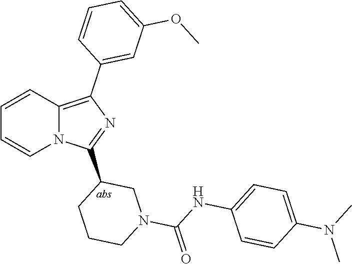 [SMILES: COc1cccc(-c2nc([C@@H]3CCCN(C(=O)Nc4ccc(N(C)C)cc4)C3)n3ccccc23)c1] | 1-2 |
| 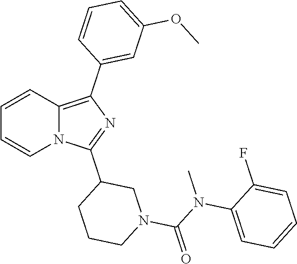 [SMILES: COc1cccc(-c2nc(C3CCCN(C(=O)N(C)c4ccccc4F)C3)n3ccccc23)c1] | 1-3 |
| 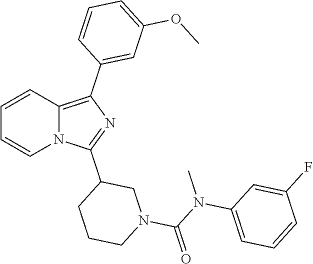 [SMILES: COc1cccc(-c2nc(C3CCCN(C(=O)N(C)c4cccc(F)c4)C3)n3ccccc23)c1] | 1-4 |
| 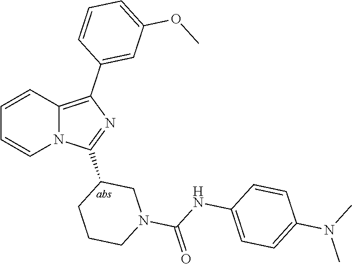 [SMILES: COc1cccc(-c2nc([C@H]3CCCN(C(=O)Nc4ccc(N(C)C)cc4)C3)n3ccccc23)c1] | 1-5 |
| 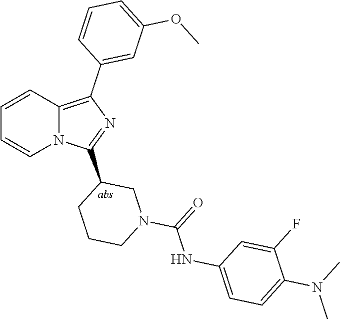 [SMILES: COc1cccc(-c2nc([C@@H]3CCCN(C(=O)Nc4ccc(N(C)C)c(F)c4)C3)n3ccccc23)c1] | 1-6 |
| 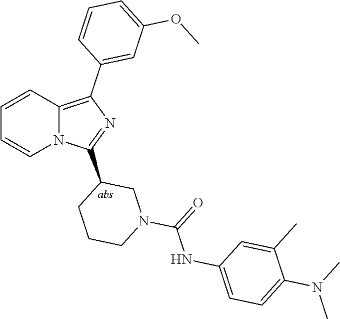 [SMILES: COc1cccc(-c2nc([C@@H]3CCCN(C(=O)Nc4ccc(N(C)C)c(C)c4)C3)n3ccccc23)c1] | 1-7 |
| 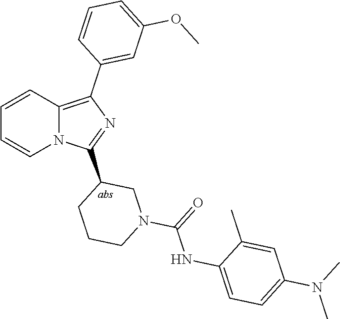 [SMILES: COc1cccc(-c2nc([C@@H]3CCCN(C(=O)Nc4ccc(N(C)C)cc4C)C3)n3ccccc23)c1] | 1-8 |
| 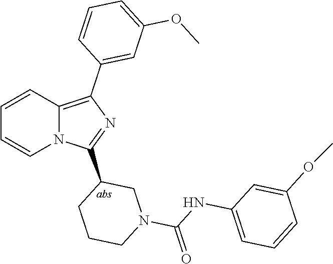 [SMILES: COc1cccc(NC(=O)N2CCC[C@@H](c3nc(-c4cccc(OC)c4)c4ccccn34)C2)c1] | 1-9 |
| 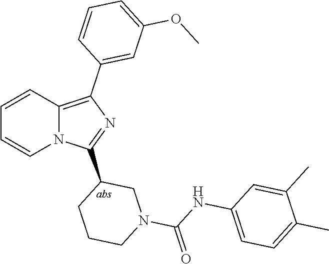 [SMILES: COc1cccc(-c2nc([C@@H]3CCCN(C(=O)Nc4ccc(C)c(C)c4)C3)n3ccccc23)c1] | 1-10 |
| 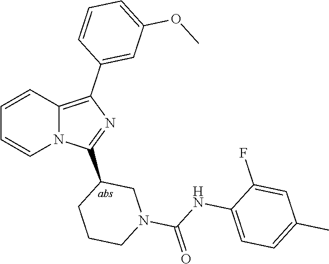 [SMILES: COc1cccc(-c2nc([C@@H]3CCCN(C(=O)Nc4ccc(C)cc4F)C3)n3ccccc23)c1] | 1-11 |
| 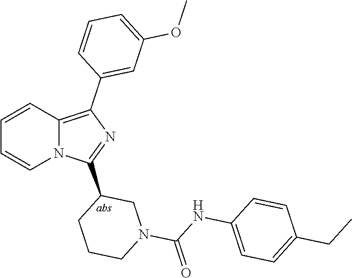 [SMILES: CCc1ccc(NC(=O)N2CCC[C@@H](c3nc(-c4cccc(OC)c4)c4ccccn34)C2)cc1] | 1-12 |
| 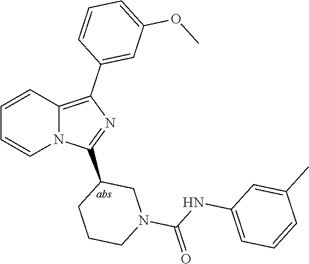 [SMILES: COc1cccc(-c2nc([C@@H]3CCCN(C(=O)Nc4cccc(C)c4)C3)n3ccccc23)c1] | 1-13 |
| 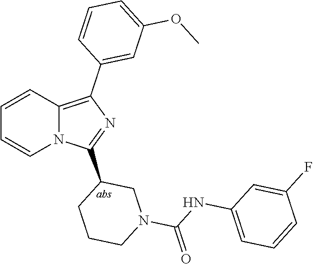 [SMILES: COc1cccc(-c2nc([C@@H]3CCCN(C(=O)Nc4cccc(F)c4)C3)n3ccccc23)c1] | 1-14 |
| 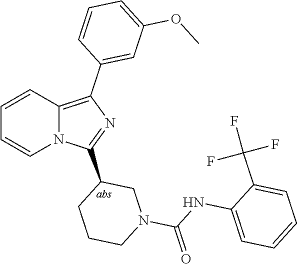 [SMILES: COc1cccc(-c2nc([C@@H]3CCCN(C(=O)Nc4ccccc4C(F)(F)F)C3)n3ccccc23)c1] | 1-15 |
| 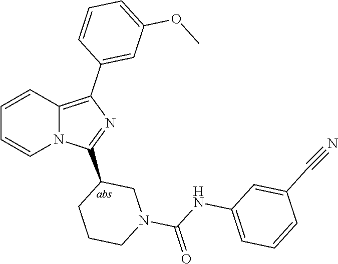 [SMILES: COc1cccc(-c2nc([C@@H]3CCCN(C(=O)Nc4cccc(C#N)c4)C3)n3ccccc23)c1] | 1-16 |
| 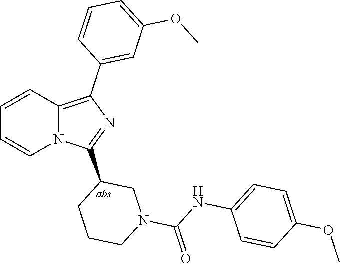 [SMILES: COc1ccc(NC(=O)N2CCC[C@@H](c3nc(-c4cccc(OC)c4)c4ccccn34)C2)cc1] | 1-17 |
| 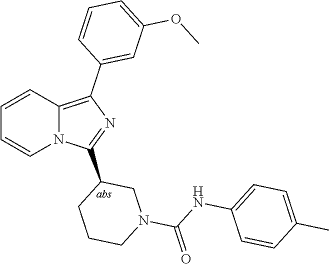 [SMILES: COc1cccc(-c2nc([C@@H]3CCCN(C(=O)Nc4ccc(C)cc4)C3)n3ccccc23)c1] | 1-18 |
| 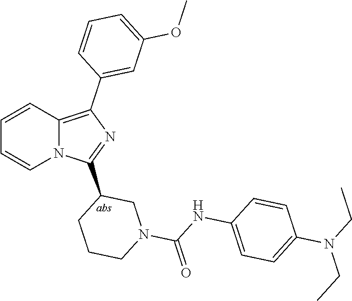 [SMILES: CCN(CC)c1ccc(NC(=O)N2CCC[C@@H](c3nc(-c4cccc(OC)c4)c4ccccn34)C2)cc1] | 1-19 |
| 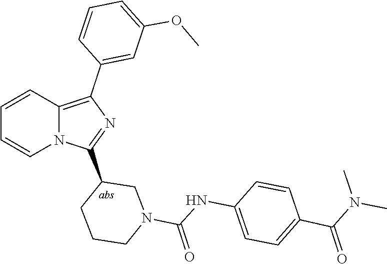 [SMILES: COc1cccc(-c2nc([C@@H]3CCCN(C(=O)Nc4ccc(C(=O)N(C)C)cc4)C3)n3ccccc23)c1] | 1-20 |
| 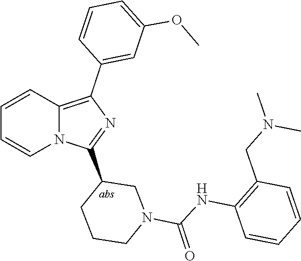 [SMILES: COc1cccc(-c2nc([C@@H]3CCCN(C(=O)Nc4ccccc4CN(C)C)C3)n3ccccc23)c1] | 1-21 |
| 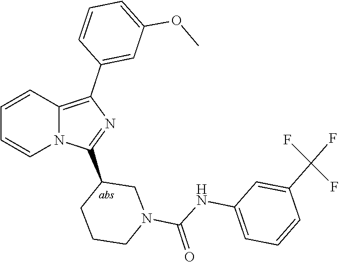 [SMILES: COc1cccc(-c2nc([C@@H]3CCCN(C(=O)Nc4cccc(C(F)(F)F)c4)C3)n3ccccc23)c1] | 1-22 |
| 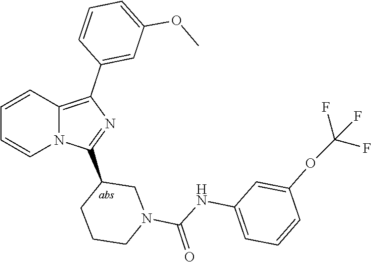 [SMILES: COc1cccc(-c2nc([C@@H]3CCCN(C(=O)Nc4cccc(OC(F)(F)F)c4)C3)n3ccccc23)c1] | 1-23 |
| 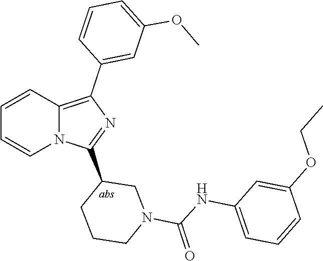 [SMILES: CCOc1cccc(NC(=O)N2CCC[C@@H](c3nc(-c4cccc(OC)c4)c4ccccn34)C2)c1] | 1-24 |
| 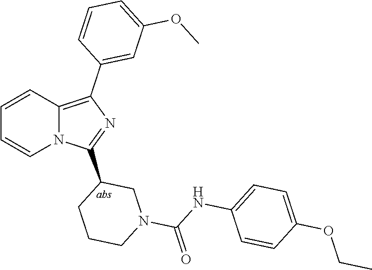 [SMILES: CCOc1ccc(NC(=O)N2CCC[C@@H](c3nc(-c4cccc(OC)c4)c4ccccn34)C2)cc1] | 1-25 |
| 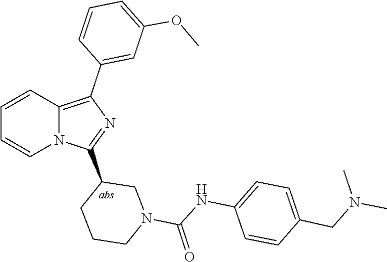 [SMILES: COc1cccc(-c2nc([C@@H]3CCCN(C(=O)Nc4ccc(CN(C)C)cc4)C3)n3ccccc23)c1] | 1-26 |
| 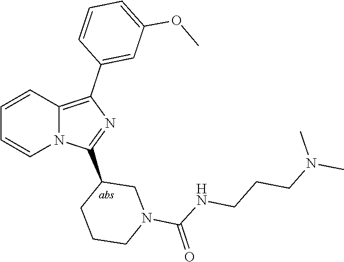 [SMILES: COc1cccc(-c2nc([C@@H]3CCCN(C(=O)NCCCN(C)C)C3)n3ccccc23)c1] | 1-27 |
| 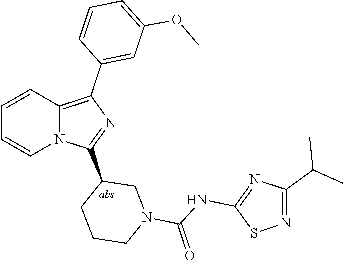 [SMILES: COc1cccc(-c2nc([C@@H]3CCCN(C(=O)Nc4nc(C(C)C)ns4)C3)n3ccccc23)c1] | 1-28 |
| 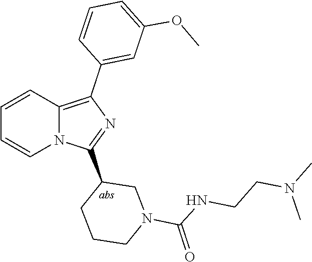 [SMILES: COc1cccc(-c2nc([C@@H]3CCCN(C(=O)NCCN(C)C)C3)n3ccccc23)c1] | 1-29 |
| 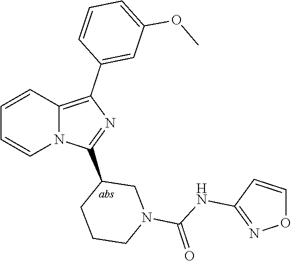 [SMILES: COc1cccc(-c2nc([C@@H]3CCCN(C(=O)Nc4ccon4)C3)n3ccccc23)c1] | 1-30 |
| 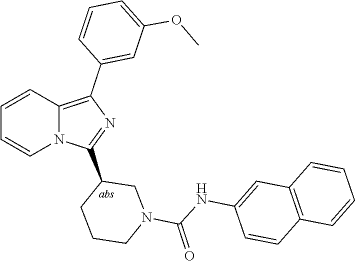 [SMILES: COc1cccc(-c2nc([C@@H]3CCCN(C(=O)Nc4ccc5ccccc5c4)C3)n3ccccc23)c1] | 1-31 |
| 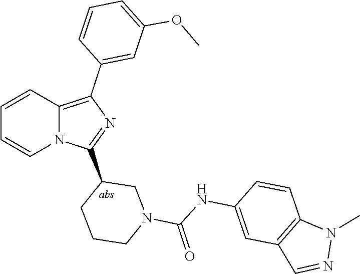 [SMILES: COc1cccc(-c2nc([C@@H]3CCCN(C(=O)Nc4ccc5c(cnn5C)c4)C3)n3ccccc23)c1] | 1-32 |
| 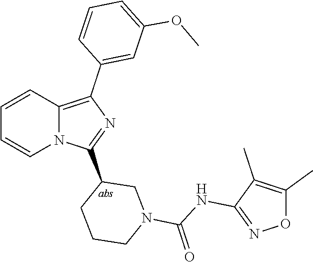 [SMILES: COc1cccc(-c2nc([C@@H]3CCCN(C(=O)Nc4noc(C)c4C)C3)n3ccccc23)c1] | 1-33 |
| 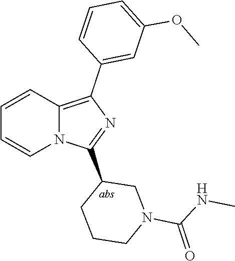 [SMILES: CNC(=O)N1CCC[C@@H](c2nc(-c3cccc(OC)c3)c3ccccn23)C1] | 2-1 |
| 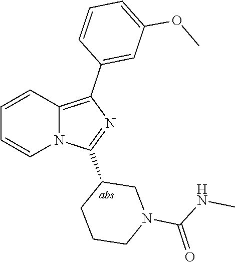 [SMILES: CNC(=O)N1CCC[C@H](c2nc(-c3cccc(OC)c3)c3ccccn23)C1] | 2-2 |
| 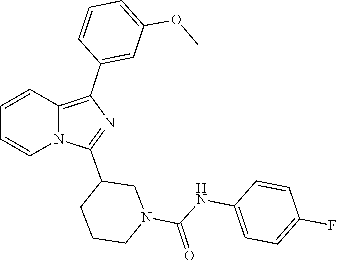 [SMILES: COc1cccc(-c2nc(C3CCCN(C(=O)Nc4ccc(F)cc4)C3)n3ccccc23)c1] | 3-1 |
| 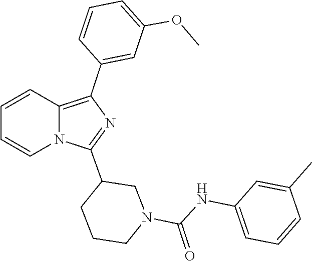 [SMILES: COc1cccc(-c2nc(C3CCCN(C(=O)Nc4cccc(C)c4)C3)n3ccccc23)c1] | 3-2 |
| 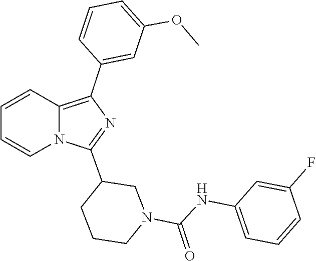 [SMILES: COc1cccc(-c2nc(C3CCCN(C(=O)Nc4cccc(F)c4)C3)n3ccccc23)c1] | 3-3 |
| 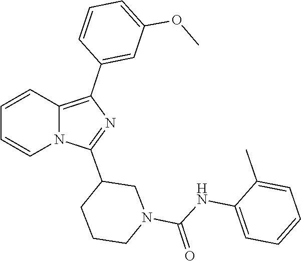 [SMILES: COc1cccc(-c2nc(C3CCCN(C(=O)Nc4ccccc4C)C3)n3ccccc23)c1] | 3-4 |
| 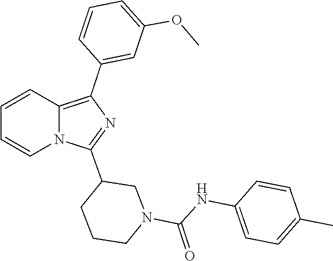 [SMILES: COc1cccc(-c2nc(C3CCCN(C(=O)Nc4ccc(C)cc4)C3)n3ccccc23)c1] | 3-5 |
| 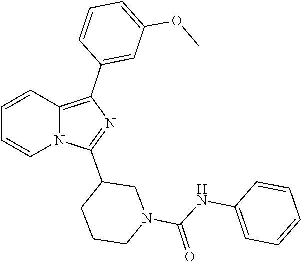 [SMILES: COc1cccc(-c2nc(C3CCCN(C(=O)Nc4ccccc4)C3)n3ccccc23)c1] | 3-6 |
| 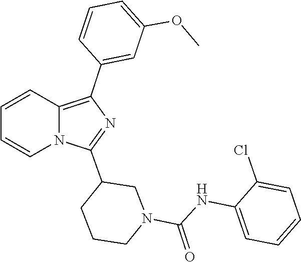 [SMILES: COc1cccc(-c2nc(C3CCCN(C(=O)Nc4ccccc4Cl)C3)n3ccccc23)c1] | 3-7 |
| 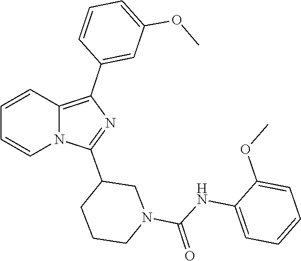 [SMILES: COc1cccc(-c2nc(C3CCCN(C(=O)Nc4ccccc4OC)C3)n3ccccc23)c1] | 3-8 |
| 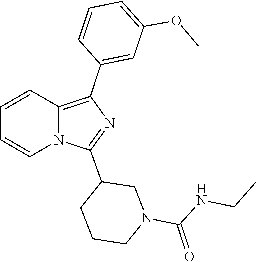 [SMILES: CCNC(=O)N1CCCC(c2nc(-c3cccc(OC)c3)c3ccccn23)C1] | 3-9 |
| 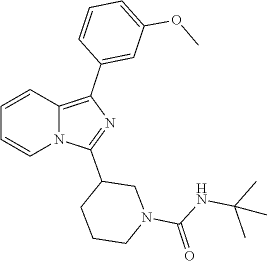 [SMILES: COc1cccc(-c2nc(C3CCCN(C(=O)NC(C)(C)C)C3)n3ccccc23)c1] | 3-10 |
| 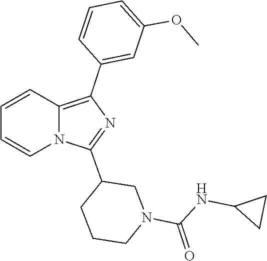 [SMILES: COc1cccc(-c2nc(C3CCCN(C(=O)NC4CC4)C3)n3ccccc23)c1] | 3-11 |
| 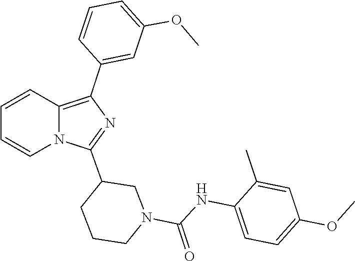 [SMILES: COc1cccc(-c2nc(C3CCCN(C(=O)Nc4ccc(OC)cc4C)C3)n3ccccc23)c1] | 3-12 |
| 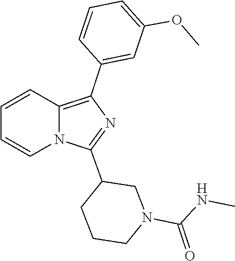 [SMILES: CNC(=O)N1CCCC(c2nc(-c3cccc(OC)c3)c3ccccn23)C1] | 3-13 |
| 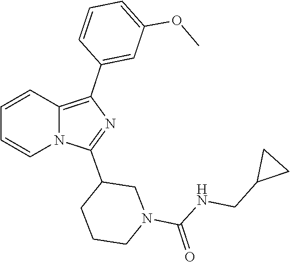 [SMILES: COc1cccc(-c2nc(C3CCCN(C(=O)NCC4CC4)C3)n3ccccc23)c1] | 3-14 |
| 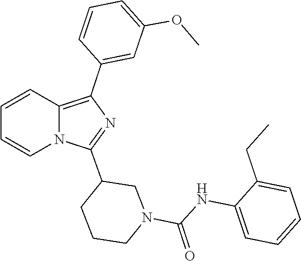 [SMILES: CCc1ccccc1NC(=O)N1CCCC(c2nc(-c3cccc(OC)c3)c3ccccn23)C1] | 3-15 |
| 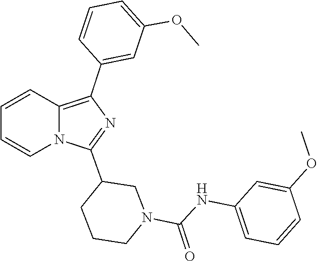 [SMILES: COc1cccc(NC(=O)N2CCCC(c3nc(-c4cccc(OC)c4)c4ccccn34)C2)c1] | 3-16 |
| 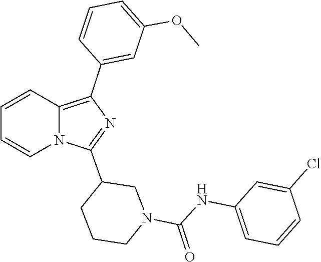 [SMILES: COc1cccc(-c2nc(C3CCCN(C(=O)Nc4cccc(Cl)c4)C3)n3ccccc23)c1] | 3-17 |
| 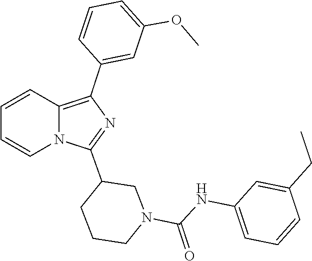 [SMILES: CCc1cccc(NC(=O)N2CCCC(c3nc(-c4cccc(OC)c4)c4ccccn34)C2)c1] | 3-18 |
| 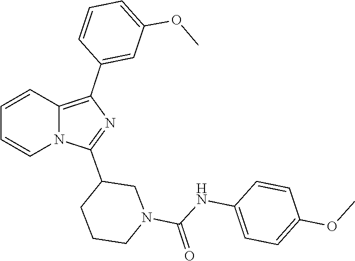 [SMILES: COc1ccc(NC(=O)N2CCCC(c3nc(-c4cccc(OC)c4)c4ccccn34)C2)cc1] | 3-19 |
| 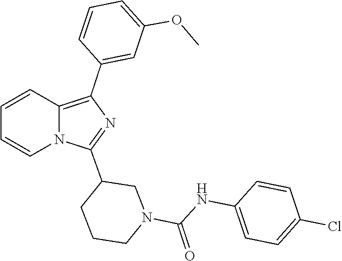 [SMILES: COc1cccc(-c2nc(C3CCCN(C(=O)Nc4ccc(Cl)cc4)C3)n3ccccc23)c1] | 3-20 |
| 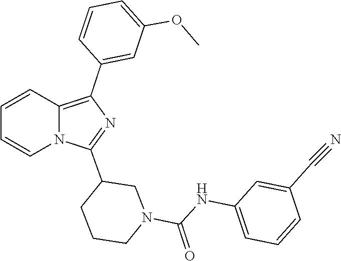 [SMILES: COc1cccc(-c2nc(C3CCCN(C(=O)Nc4cccc(C#N)c4)C3)n3ccccc23)c1] | 3-21 |
| 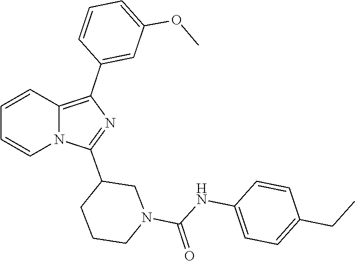 [SMILES: CCc1ccc(NC(=O)N2CCCC(c3nc(-c4cccc(OC)c4)c4ccccn34)C2)cc1] | 3-22 |
| 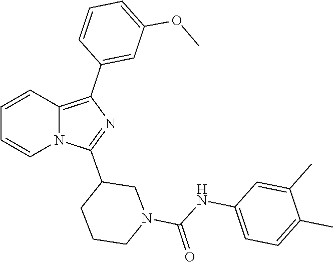 [SMILES: COc1cccc(-c2nc(C3CCCN(C(=O)Nc4ccc(C)c(C)c4)C3)n3ccccc23)c1] | 3-23 |
| 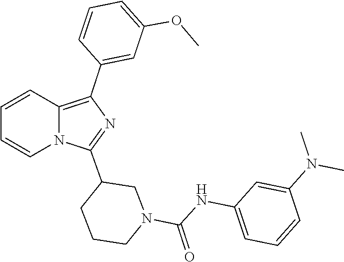 [SMILES: COc1cccc(-c2nc(C3CCCN(C(=O)Nc4cccc(N(C)C)c4)C3)n3ccccc23)c1] | 3-24 |
| 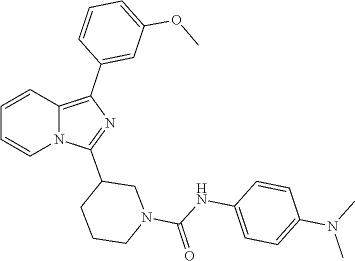 [SMILES: COc1cccc(-c2nc(C3CCCN(C(=O)Nc4ccc(N(C)C)cc4)C3)n3ccccc23)c1] | 3-25 |
| 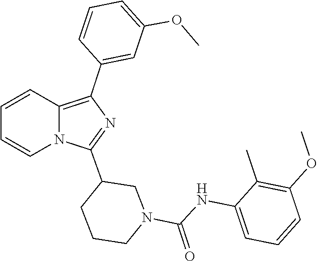 [SMILES: COc1cccc(-c2nc(C3CCCN(C(=O)Nc4cccc(OC)c4C)C3)n3ccccc23)c1] | 3-26 |
| 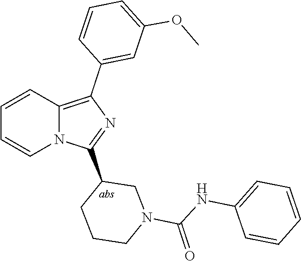 [SMILES: COc1cccc(-c2nc([C@@H]3CCCN(C(=O)Nc4ccccc4)C3)n3ccccc23)c1] | 3-27 |
| 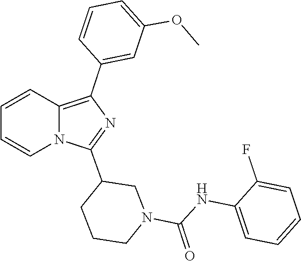 [SMILES: COc1cccc(-c2nc(C3CCCN(C(=O)Nc4ccccc4F)C3)n3ccccc23)c1] | 3-28 |
| 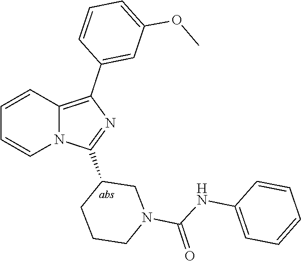 [SMILES: COc1cccc(-c2nc([C@H]3CCCN(C(=O)Nc4ccccc4)C3)n3ccccc23)c1] | 3-29 |
| 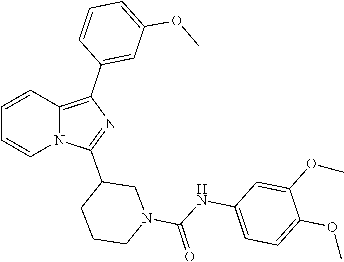 [SMILES: COc1cccc(-c2nc(C3CCCN(C(=O)Nc4ccc(OC)c(OC)c4)C3)n3ccccc23)c1] | 4-1 |
| 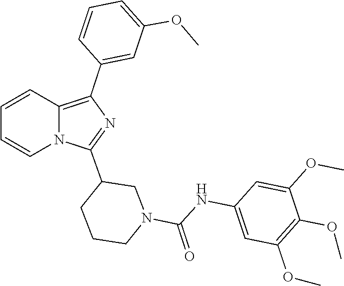 [SMILES: COc1cccc(-c2nc(C3CCCN(C(=O)Nc4cc(OC)c(OC)c(OC)c4)C3)n3ccccc23)c1] | 4-2 |
| 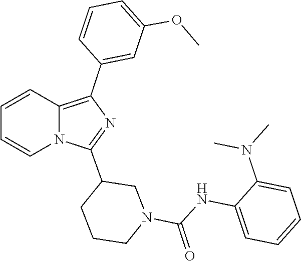 [SMILES: COc1cccc(-c2nc(C3CCCN(C(=O)Nc4ccccc4N(C)C)C3)n3ccccc23)c1] | 4-3 |
| 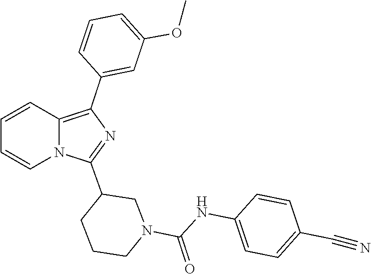 [SMILES: COc1cccc(-c2nc(C3CCCN(C(=O)Nc4ccc(C#N)cc4)C3)n3ccccc23)c1] | 4-4 |
| 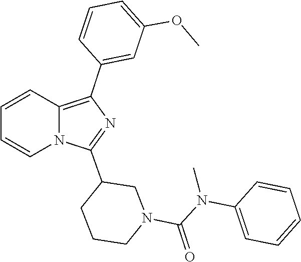 [SMILES: COc1cccc(-c2nc(C3CCCN(C(=O)N(C)c4ccccc4)C3)n3ccccc23)c1] | 5-1 |
| 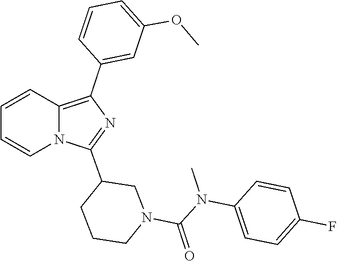 [SMILES: COc1cccc(-c2nc(C3CCCN(C(=O)N(C)c4ccc(F)cc4)C3)n3ccccc23)c1] | 5-2 |
| 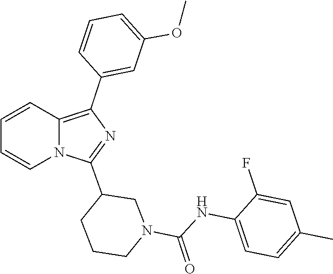 [SMILES: COc1cccc(-c2nc(C3CCCN(C(=O)Nc4ccc(C)cc4F)C3)n3ccccc23)c1] | 5-3 |
| 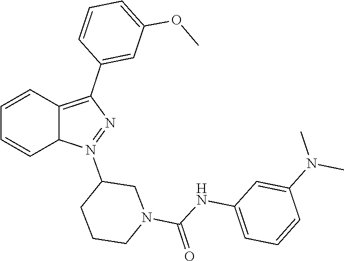 | 5-4 |
| 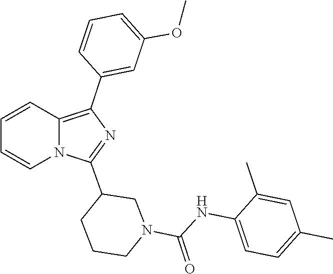 [SMILES: COc1cccc(-c2nc(C3CCCN(C(=O)Nc4ccc(C)cc4C)C3)n3ccccc23)c1] | 5-5 |
| 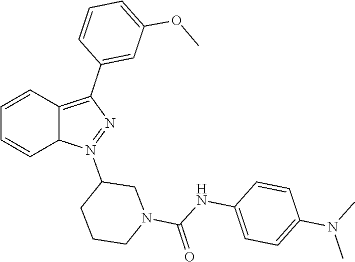 | 5-6 |
| 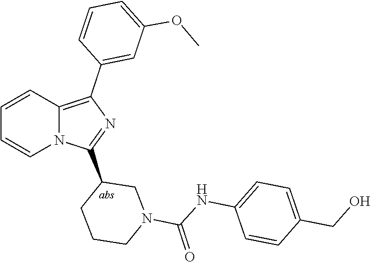 [SMILES: COc1cccc(-c2nc([C@@H]3CCCN(C(=O)Nc4ccc(CO)cc4)C3)n3ccccc23)c1] | 5-7 |
| 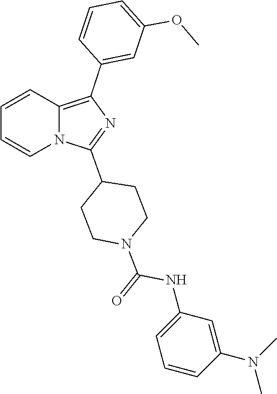 [SMILES: COc1cccc(-c2nc(C3CCN(C(=O)Nc4cccc(N(C)C)c4)CC3)n3ccccc23)c1] | 5-8 |
| 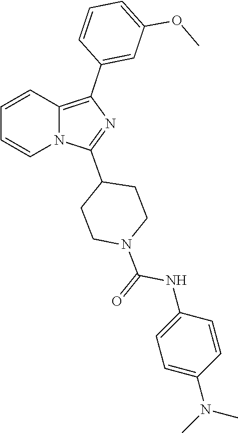 [SMILES: COc1cccc(-c2nc(C3CCN(C(=O)Nc4ccc(N(C)C)cc4)CC3)n3ccccc23)c1] | 5-9 |
| 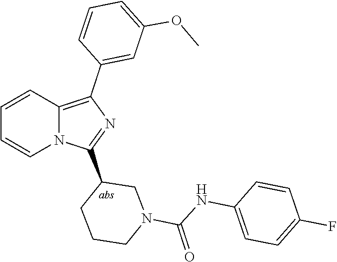 [SMILES: COc1cccc(-c2nc([C@@H]3CCCN(C(=O)Nc4ccc(F)cc4)C3)n3ccccc23)c1] | 5-10 |
| 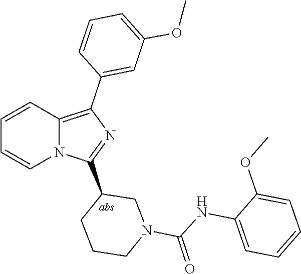 [SMILES: COc1cccc(-c2nc([C@@H]3CCCN(C(=O)Nc4ccccc4OC)C3)n3ccccc23)c1] | 5-11 |
| 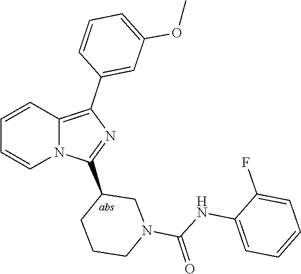 [SMILES: COc1cccc(-c2nc([C@@H]3CCCN(C(=O)Nc4ccccc4F)C3)n3ccccc23)c1] | 5-12 |
| 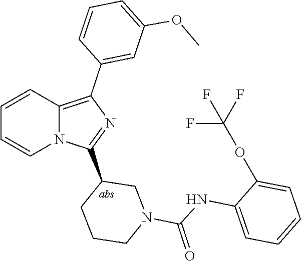 [SMILES: COc1cccc(-c2nc([C@@H]3CCCN(C(=O)Nc4ccccc4OC(F)(F)F)C3)n3ccccc23)c1] | 5-13 |
| 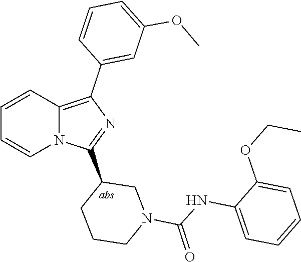 [SMILES: CCOc1ccccc1NC(=O)N1CCC[C@@H](c2nc(-c3cccc(OC)c3)c3ccccn23)C1] | 5-14 |
| 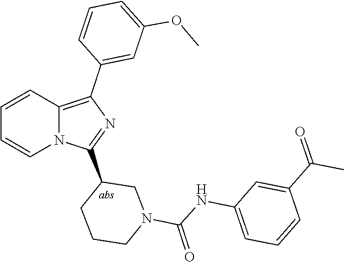 [SMILES: COc1cccc(-c2nc([C@@H]3CCCN(C(=O)Nc4cccc(C(C)=O)c4)C3)n3ccccc23)c1] | 5-15 |
| 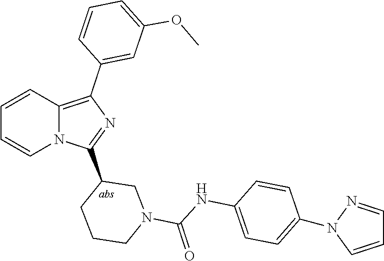 [SMILES: COc1cccc(-c2nc([C@@H]3CCCN(C(=O)Nc4ccc(-n5cccn5)cc4)C3)n3ccccc23)c1] | 5-16 |
| 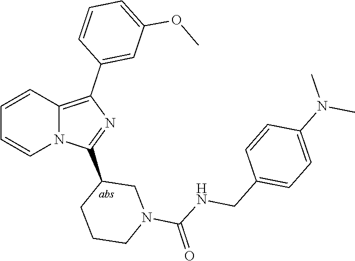 [SMILES: COc1cccc(-c2nc([C@@H]3CCCN(C(=O)NCc4ccc(N(C)C)cc4)C3)n3ccccc23)c1] | 5-17 |
| 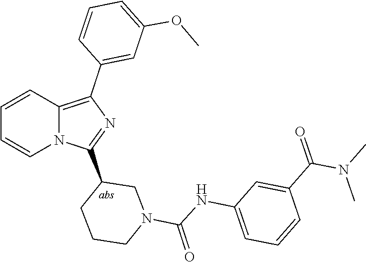 [SMILES: COc1cccc(-c2nc([C@@H]3CCCN(C(=O)Nc4cccc(C(=O)N(C)C)c4)C3)n3ccccc23)c1] | 5-18 |
| 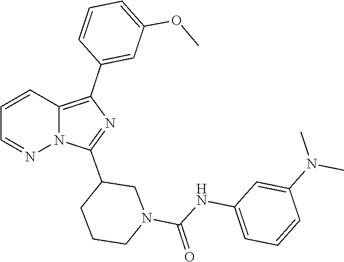 [SMILES: COc1cccc(-c2nc(C3CCCN(C(=O)Nc4cccc(N(C)C)c4)C3)n3ncccc23)c1] | 5-19 |
| 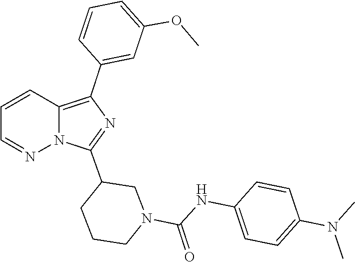 [SMILES: COc1cccc(-c2nc(C3CCCN(C(=O)Nc4ccc(N(C)C)cc4)C3)n3ncccc23)c1] | 5-20 |
| 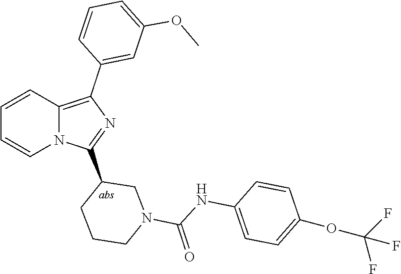 [SMILES: COc1cccc(-c2nc([C@@H]3CCCN(C(=O)Nc4ccc(OC(F)(F)F)cc4)C3)n3ccccc23)c1] | 5-21 |
| 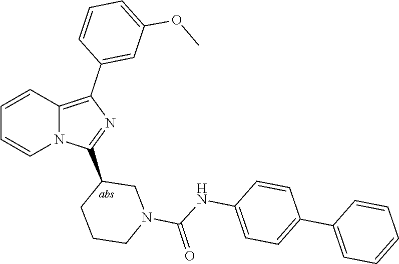 [SMILES: COc1cccc(-c2nc([C@@H]3CCCN(C(=O)Nc4ccc(-c5ccccc5)cc4)C3)n3ccccc23)c1] | 5-22 |
| 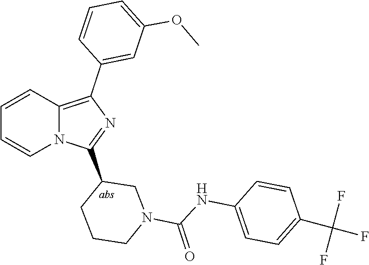 [SMILES: COc1cccc(-c2nc([C@@H]3CCCN(C(=O)Nc4ccc(C(F)(F)F)cc4)C3)n3ccccc23)c1] | 5-23 |
| 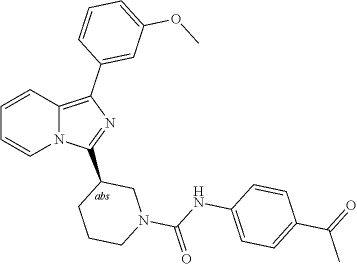 [SMILES: COc1cccc(-c2nc([C@@H]3CCCN(C(=O)Nc4ccc(C(C)=O)cc4)C3)n3ccccc23)c1] | 5-24 |
| 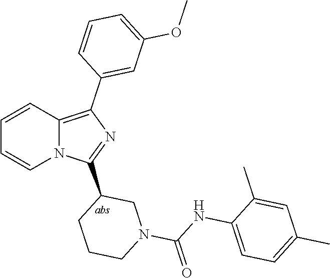 [SMILES: COc1cccc(-c2nc([C@@H]3CCCN(C(=O)Nc4ccc(C)cc4C)C3)n3ccccc23)c1] | 5-25 |
| 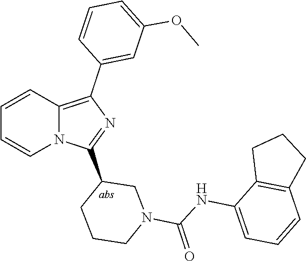 [SMILES: COc1cccc(-c2nc([C@@H]3CCCN(C(=O)Nc4cccc5c4CCC5)C3)n3ccccc23)c1] | 5-26 |
| 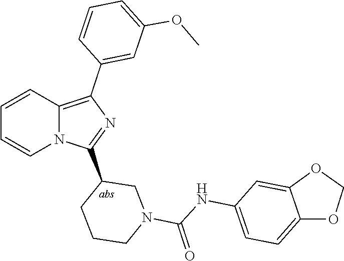 [SMILES: COc1cccc(-c2nc([C@@H]3CCCN(C(=O)Nc4ccc5c(c4)OCO5)C3)n3ccccc23)c1] | 5-27 |
| 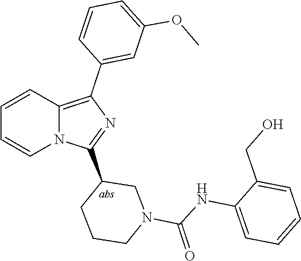 [SMILES: COc1cccc(-c2nc([C@@H]3CCCN(C(=O)Nc4ccccc4CO)C3)n3ccccc23)c1] | 5-28 |
| 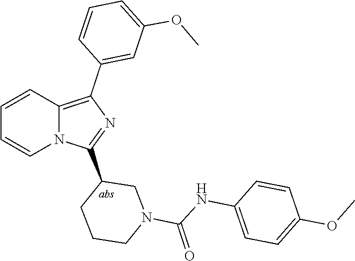 [SMILES: COc1ccc(NC(=O)N2CCC[C@@H](c3nc(-c4cccc(OC)c4)c4ccccn34)C2)cc1] | 5-29 |
| 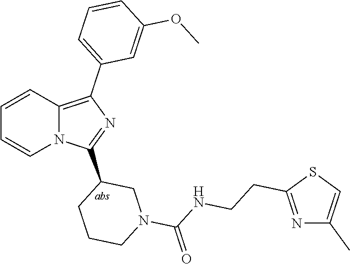 [SMILES: COc1cccc(-c2nc([C@@H]3CCCN(C(=O)NCCc4nc(C)cs4)C3)n3ccccc23)c1] | 5-30 |
| 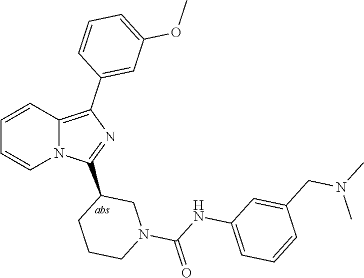 [SMILES: COc1cccc(-c2nc([C@@H]3CCCN(C(=O)Nc4cccc(CN(C)C)c4)C3)n3ccccc23)c1] | 5-31 |
| 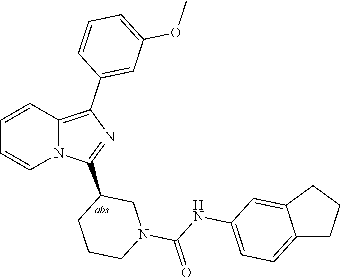 [SMILES: COc1cccc(-c2nc([C@@H]3CCCN(C(=O)Nc4ccc5c(c4)CCC5)C3)n3ccccc23)c1] | 5-32 |
| 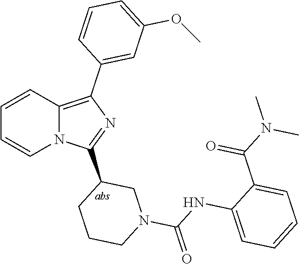 [SMILES: COc1cccc(-c2nc([C@@H]3CCCN(C(=O)Nc4ccccc4C(=O)N(C)C)C3)n3ccccc23)c1] | 5-33 |
| 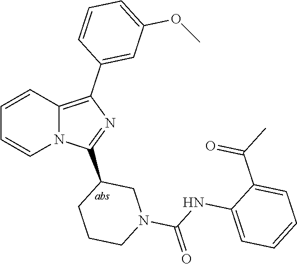 [SMILES: COc1cccc(-c2nc([C@@H]3CCCN(C(=O)Nc4ccccc4C(C)=O)C3)n3ccccc23)c1] | 5-34 |
| 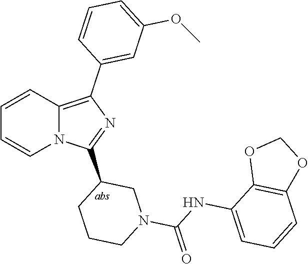 [SMILES: COc1cccc(-c2nc([C@@H]3CCCN(C(=O)Nc4cccc5c4OCO5)C3)n3ccccc23)c1] | 5-35 |
| 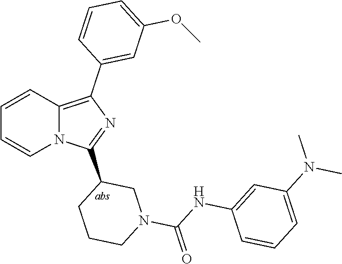 [SMILES: COc1cccc(-c2nc([C@@H]3CCCN(C(=O)Nc4cccc(N(C)C)c4)C3)n3ccccc23)c1] | 5-36 |
| 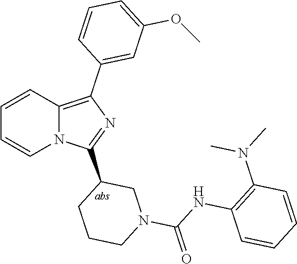 [SMILES: COc1cccc(-c2nc([C@@H]3CCCN(C(=O)Nc4ccccc4N(C)C)C3)n3ccccc23)c1] | 5-37 |
| 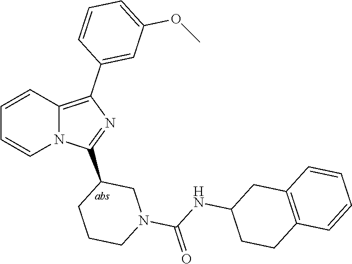 [SMILES: COc1cccc(-c2nc([C@@H]3CCCN(C(=O)NC4CCc5ccccc5C4)C3)n3ccccc23)c1] | 5-38 |
| 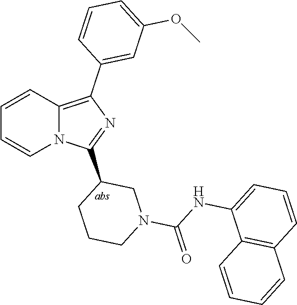 [SMILES: COc1cccc(-c2nc([C@@H]3CCCN(C(=O)Nc4cccc5ccccc45)C3)n3ccccc23)c1] | 5-39 |
| 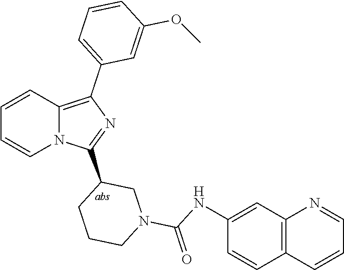 [SMILES: COc1cccc(-c2nc([C@@H]3CCCN(C(=O)Nc4ccc5cccnc5c4)C3)n3ccccc23)c1] | 5-40 |
| 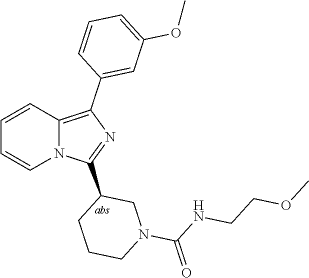 [SMILES: COCCNC(=O)N1CCC[C@@H](c2nc(-c3cccc(OC)c3)c3ccccn23)C1] | 5-41 |
| 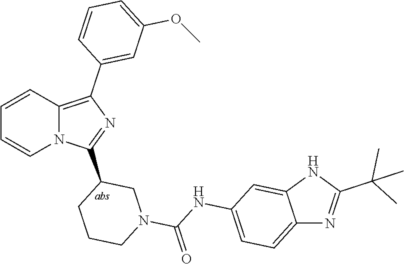 [SMILES: COc1cccc(-c2nc([C@@H]3CCCN(C(=O)Nc4ccc5nc(C(C)(C)C)[nH]c5c4)C3)n3ccccc23)c1] | 5-42 |
| 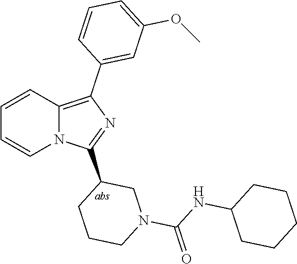 [SMILES: COc1cccc(-c2nc([C@@H]3CCCN(C(=O)NC4CCCCC4)C3)n3ccccc23)c1] | 5-43 |
| 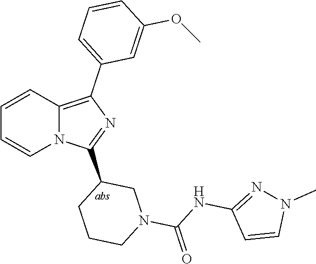 [SMILES: COc1cccc(-c2nc([C@@H]3CCCN(C(=O)Nc4ccn(C)n4)C3)n3ccccc23)c1] | 5-44 |
| 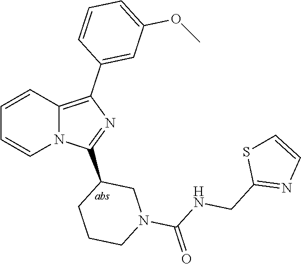 [SMILES: COc1cccc(-c2nc([C@@H]3CCCN(C(=O)NCc4nccs4)C3)n3ccccc23)c1] | 5-45 |
| 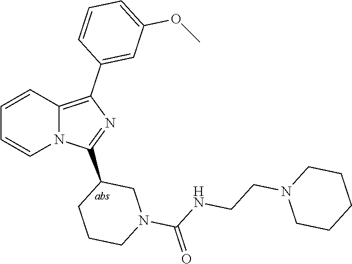 [SMILES: COc1cccc(-c2nc([C@@H]3CCCN(C(=O)NCCN4CCCCC4)C3)n3ccccc23)c1] | 5-46 |
| 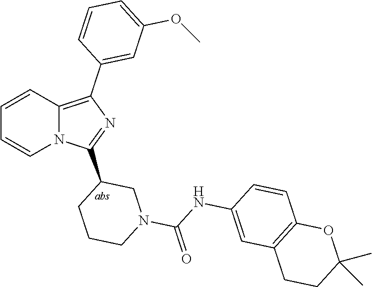 [SMILES: COc1cccc(-c2nc([C@@H]3CCCN(C(=O)Nc4ccc5c(c4)CCC(C)(C)O5)C3)n3ccccc23)c1] | 5-47 |
| 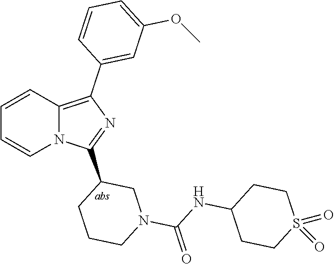 [SMILES: COc1cccc(-c2nc([C@@H]3CCCN(C(=O)NC4CCS(=O)(=O)CC4)C3)n3ccccc23)c1] | 5-48 |
| 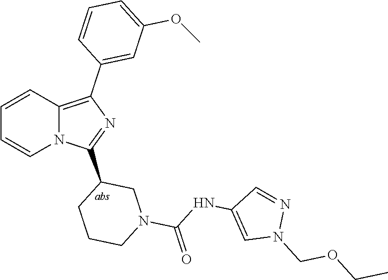 [SMILES: CCOCn1cc(NC(=O)N2CCC[C@@H](c3nc(-c4cccc(OC)c4)c4ccccn34)C2)cn1] | 5-49 |
| 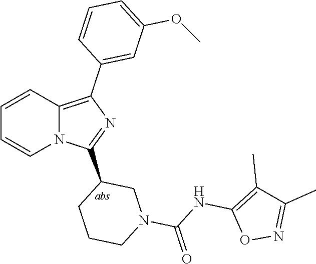 [SMILES: COc1cccc(-c2nc([C@@H]3CCCN(C(=O)Nc4onc(C)c4C)C3)n3ccccc23)c1] | 5-50 |
| 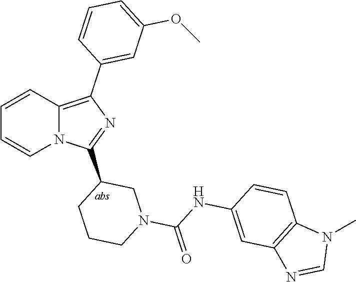 [SMILES: COc1cccc(-c2nc([C@@H]3CCCN(C(=O)Nc4ccc5c(c4)ncn5C)C3)n3ccccc23)c1] | 5-51 |
| 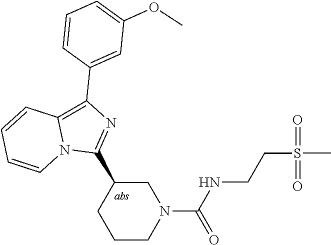 [SMILES: COc1cccc(-c2nc([C@@H]3CCCN(C(=O)NCCS(C)(=O)=O)C3)n3ccccc23)c1] | 5-52 |
| 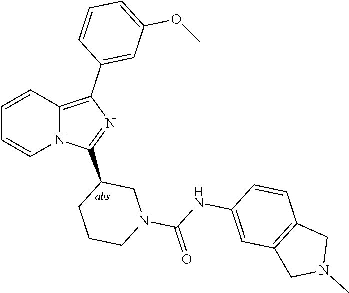 [SMILES: COc1cccc(-c2nc([C@@H]3CCCN(C(=O)Nc4ccc5c(c4)CN(C)C5)C3)n3ccccc23)c1] | 5-53 |
| 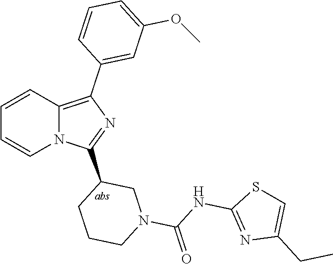 [SMILES: CCc1csc(NC(=O)N2CCC[C@@H](c3nc(-c4cccc(OC)c4)c4ccccn34)C2)n1] | 5-54 |
| 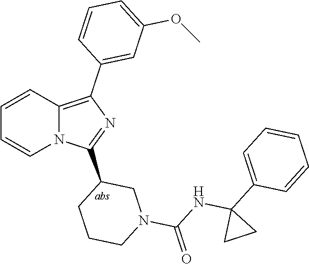 [SMILES: COc1cccc(-c2nc([C@@H]3CCCN(C(=O)NC4(c5ccccc5)CC4)C3)n3ccccc23)c1] | 5-55 |
| 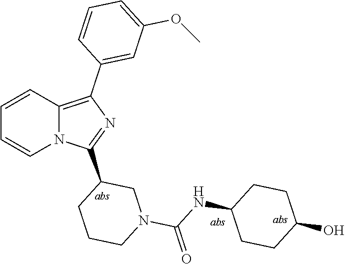 [SMILES: COc1cccc(-c2nc([C@@H]3CCCN(C(=O)N[C@H]4CC[C@@H](O)CC4)C3)n3ccccc23)c1] | 5-56 |
| 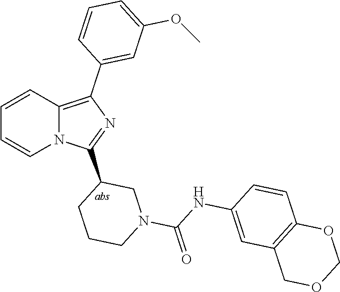 [SMILES: COc1cccc(-c2nc([C@@H]3CCCN(C(=O)Nc4ccc5c(c4)COCO5)C3)n3ccccc23)c1] | 5-57 |
| 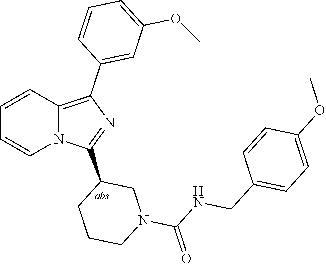 [SMILES: COc1ccc(CNC(=O)N2CCC[C@@H](c3nc(-c4cccc(OC)c4)c4ccccn34)C2)cc1] | 5-58 |
| 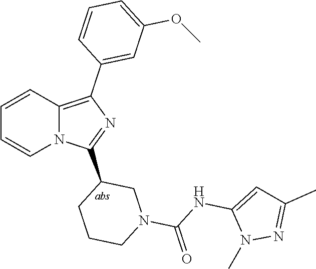 [SMILES: COc1cccc(-c2nc([C@@H]3CCCN(C(=O)Nc4cc(C)nn4C)C3)n3ccccc23)c1] | 5-59 |
| 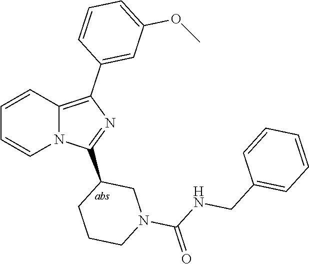 [SMILES: COc1cccc(-c2nc([C@@H]3CCCN(C(=O)NCc4ccccc4)C3)n3ccccc23)c1] | 5-60 |
| 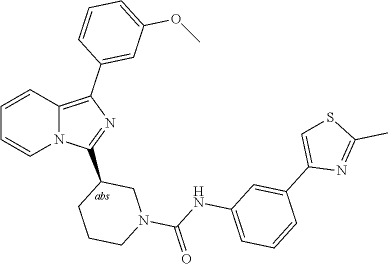 [SMILES: COc1cccc(-c2nc([C@@H]3CCCN(C(=O)Nc4cccc(-c5csc(C)n5)c4)C3)n3ccccc23)c1] | 5-61 |
| 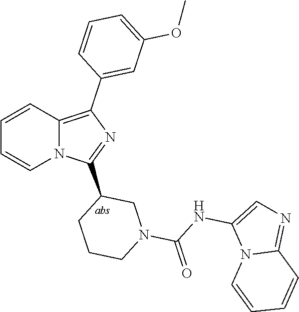 [SMILES: COc1cccc(-c2nc([C@@H]3CCCN(C(=O)Nc4cnc5ccccn45)C3)n3ccccc23)c1] | 5-62 |
| 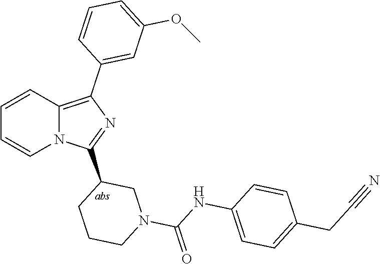 [SMILES: COc1cccc(-c2nc([C@@H]3CCCN(C(=O)Nc4ccc(CC#N)cc4)C3)n3ccccc23)c1] | 5-63 |
| 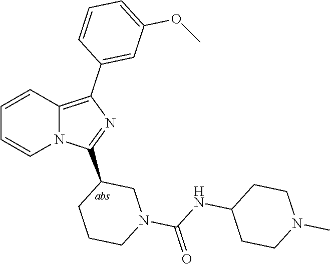 [SMILES: COc1cccc(-c2nc([C@@H]3CCCN(C(=O)NC4CCN(C)CC4)C3)n3ccccc23)c1] | 5-64 |
| 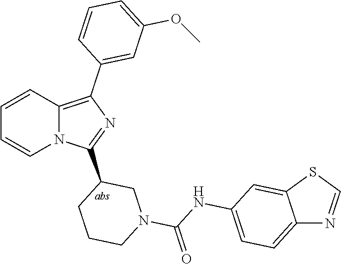 [SMILES: COc1cccc(-c2nc([C@@H]3CCCN(C(=O)Nc4ccc5ncsc5c4)C3)n3ccccc23)c1] | 5-65 |
| 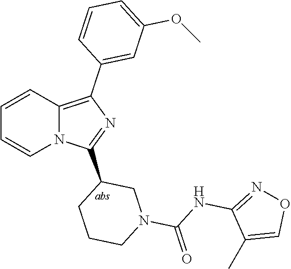 [SMILES: COc1cccc(-c2nc([C@@H]3CCCN(C(=O)Nc4nocc4C)C3)n3ccccc23)c1] | 5-66 |
| 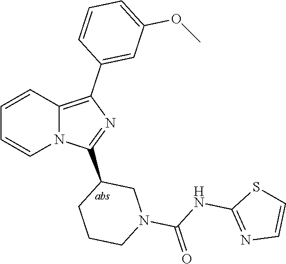 [SMILES: COc1cccc(-c2nc([C@@H]3CCCN(C(=O)Nc4nccs4)C3)n3ccccc23)c1] | 5-67 |
| 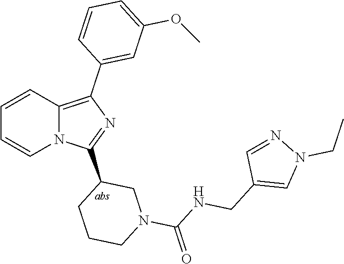 [SMILES: CCn1cc(CNC(=O)N2CCC[C@@H](c3nc(-c4cccc(OC)c4)c4ccccn34)C2)cn1] | 5-68 |
| 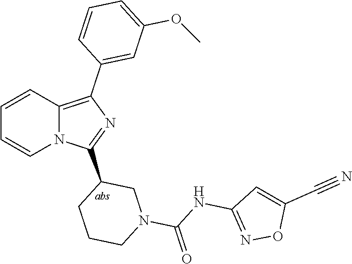 [SMILES: COc1cccc(-c2nc([C@@H]3CCCN(C(=O)Nc4cc(C#N)on4)C3)n3ccccc23)c1] | 5-69 |
| 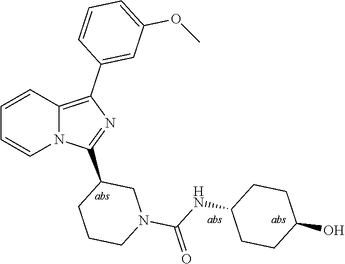 [SMILES: COc1cccc(-c2nc([C@@H]3CCCN(C(=O)N[C@H]4CC[C@H](O)CC4)C3)n3ccccc23)c1] | 5-70 |
| 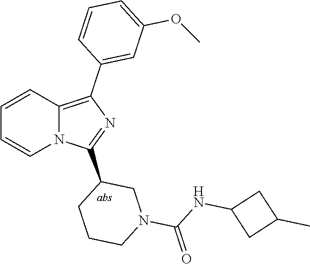 [SMILES: COc1cccc(-c2nc([C@@H]3CCCN(C(=O)NC4CC(C)C4)C3)n3ccccc23)c1] | 5-71 |
| 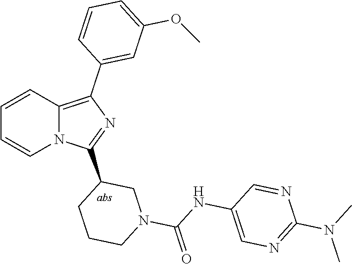 [SMILES: COc1cccc(-c2nc([C@@H]3CCCN(C(=O)Nc4cnc(N(C)C)nc4)C3)n3ccccc23)c1] | 5-72 |
| 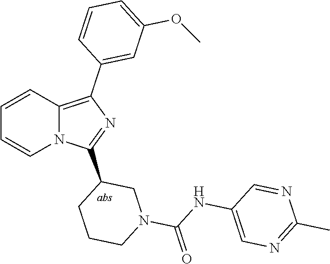 [SMILES: COc1cccc(-c2nc([C@@H]3CCCN(C(=O)Nc4cnc(C)nc4)C3)n3ccccc23)c1] | 5-73 |
| 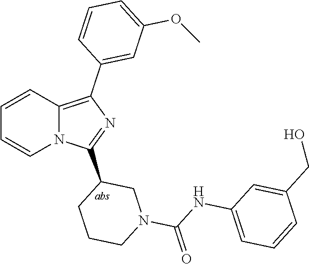 [SMILES: COc1cccc(-c2nc([C@@H]3CCCN(C(=O)Nc4cccc(CO)c4)C3)n3ccccc23)c1] | 6-1 |
| 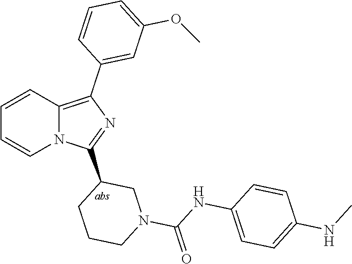 [SMILES: CNc1ccc(NC(=O)N2CCC[C@@H](c3nc(-c4cccc(OC)c4)c4ccccn34)C2)cc1] | 7-1 |
| 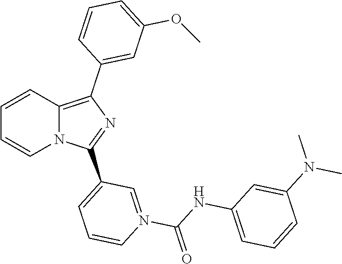 | 8-1 |
| 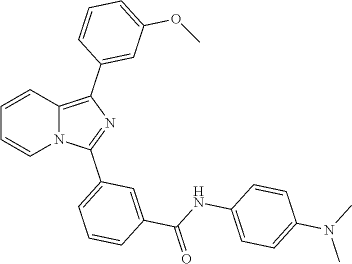 [SMILES: COc1cccc(-c2nc(-c3cccc(C(=O)Nc4ccc(N(C)C)cc4)c3)n3ccccc23)c1] | 8-2 |
| 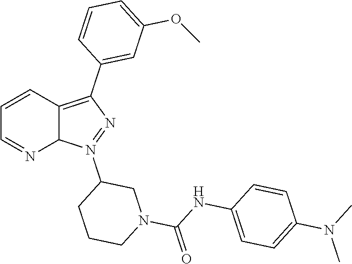 | 9-1 |
| 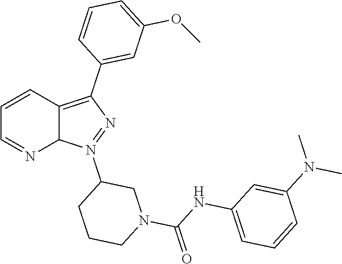 | 9-2 |
| 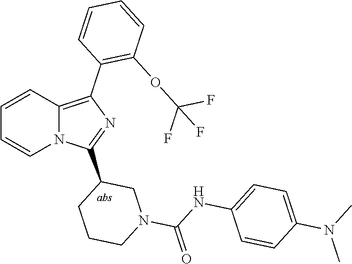 [SMILES: CN(C)c1ccc(NC(=O)N2CCC[C@@H](c3nc(-c4ccccc4OC(F)(F)F)c4ccccn34)C2)cc1] | 10-1 |
| 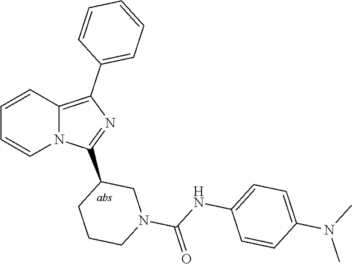 [SMILES: CN(C)c1ccc(NC(=O)N2CCC[C@@H](c3nc(-c4ccccc4)c4ccccn34)C2)cc1] | 10-2 |
| 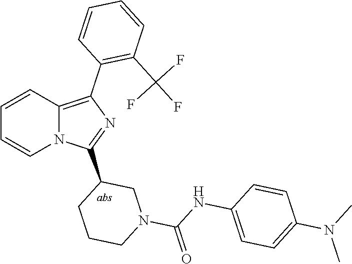 [SMILES: CN(C)c1ccc(NC(=O)N2CCC[C@@H](c3nc(-c4ccccc4C(F)(F)F)c4ccccn34)C2)cc1] | 10-3 |
| 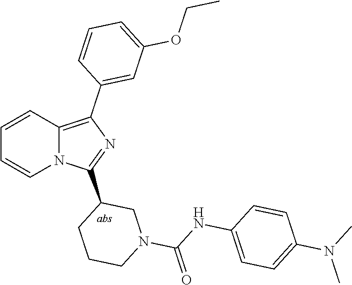 [SMILES: CCOc1cccc(-c2nc([C@@H]3CCCN(C(=O)Nc4ccc(N(C)C)cc4)C3)n3ccccc23)c1] | 10-4 |
| 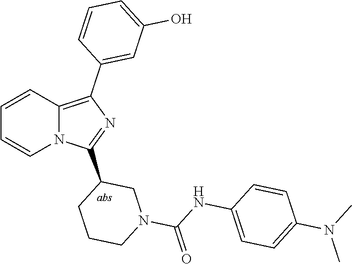 [SMILES: CN(C)c1ccc(NC(=O)N2CCC[C@@H](c3nc(-c4cccc(O)c4)c4ccccn34)C2)cc1] | 10-5 |
| 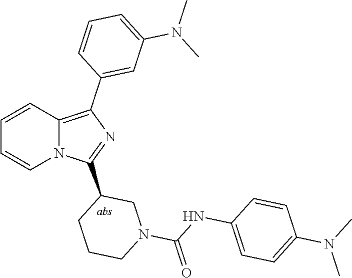 [SMILES: CN(C)c1ccc(NC(=O)N2CCC[C@@H](c3nc(-c4cccc(N(C)C)c4)c4ccccn34)C2)cc1] | 10-6 |
| 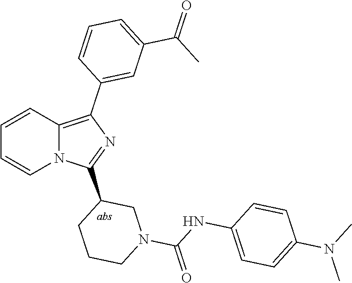 [SMILES: CC(=O)c1cccc(-c2nc([C@@H]3CCCN(C(=O)Nc4ccc(N(C)C)cc4)C3)n3ccccc23)c1] | 10-7 |
| 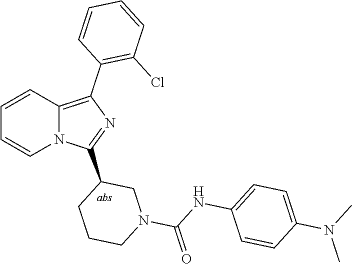 [SMILES: CN(C)c1ccc(NC(=O)N2CCC[C@@H](c3nc(-c4ccccc4Cl)c4ccccn34)C2)cc1] | 10-8 |
| 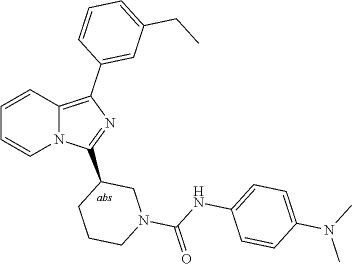 [SMILES: CCc1cccc(-c2nc([C@@H]3CCCN(C(=O)Nc4ccc(N(C)C)cc4)C3)n3ccccc23)c1] | 10-9 |
| 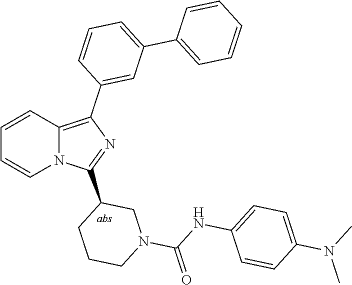 [SMILES: CN(C)c1ccc(NC(=O)N2CCC[C@@H](c3nc(-c4cccc(-c5ccccc5)c4)c4ccccn34)C2)cc1] | 10-10 |
| 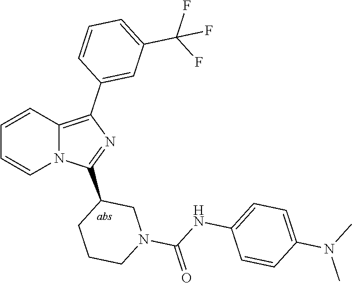 [SMILES: CN(C)c1ccc(NC(=O)N2CCC[C@@H](c3nc(-c4cccc(C(F)(F)F)c4)c4ccccn34)C2)cc1] | 10-11 |
| 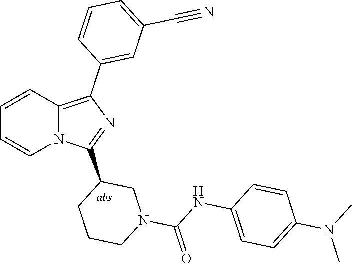 [SMILES: CN(C)c1ccc(NC(=O)N2CCC[C@@H](c3nc(-c4cccc(C#N)c4)c4ccccn34)C2)cc1] | 10-12 |
| 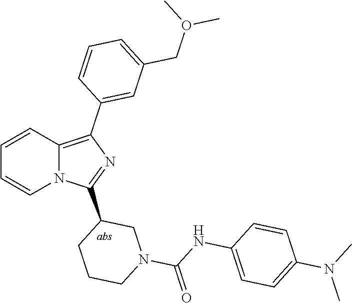 | 10-13 |
| 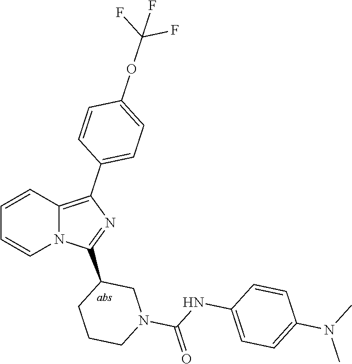 [SMILES: CN(C)c1ccc(NC(=O)N2CCC[C@@H](c3nc(-c4ccc(OC(F)(F)F)cc4)c4ccccn34)C2)cc1] | 10-14 |
| 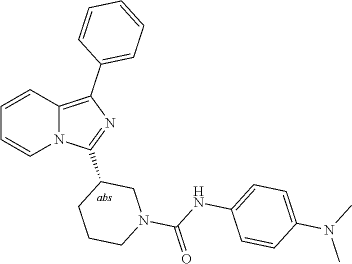 [SMILES: CN(C)c1ccc(NC(=O)N2CCC[C@H](c3nc(-c4ccccc4)c4ccccn34)C2)cc1] | 10-15 |
| 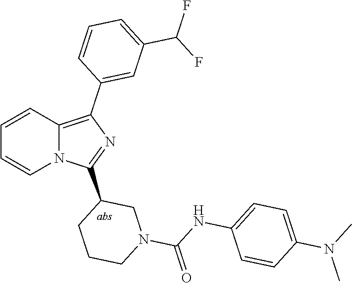 [SMILES: CN(C)c1ccc(NC(=O)N2CCC[C@@H](c3nc(-c4cccc(C(F)F)c4)c4ccccn34)C2)cc1] | 10-16 |
| 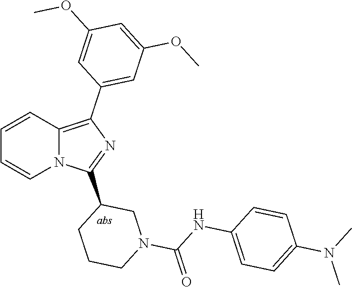 [SMILES: COc1cc(OC)cc(-c2nc([C@@H]3CCCN(C(=O)Nc4ccc(N(C)C)cc4)C3)n3ccccc23)c1] | 10-17 |
| 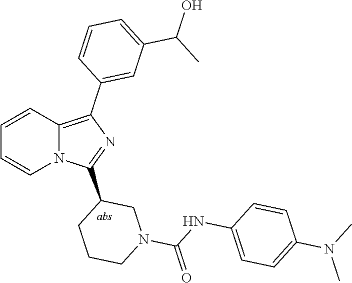 [SMILES: CC(O)c1cccc(-c2nc([C@@H]3CCCN(C(=O)Nc4ccc(N(C)C)cc4)C3)n3ccccc23)c1] | 10-18 |
| 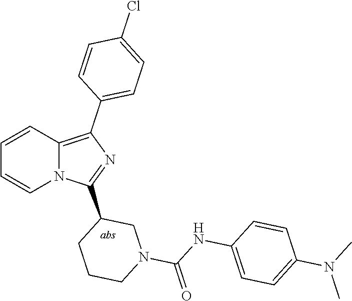 [SMILES: CN(C)c1ccc(NC(=O)N2CCC[C@@H](c3nc(-c4ccc(Cl)cc4)c4ccccn34)C2)cc1] | 10-19 |
| 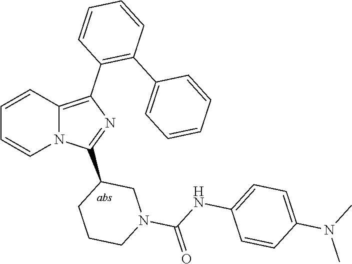 [SMILES: CN(C)c1ccc(NC(=O)N2CCC[C@@H](c3nc(-c4ccccc4-c4ccccc4)c4ccccn34)C2)cc1] | 10-20 |
| 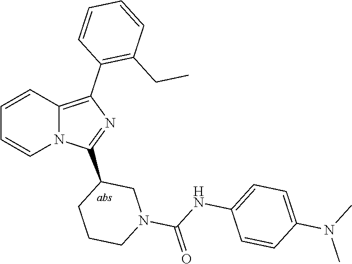 [SMILES: CCc1ccccc1-c1nc([C@@H]2CCCN(C(=O)Nc3ccc(N(C)C)cc3)C2)n2ccccc12] | 10-21 |
| 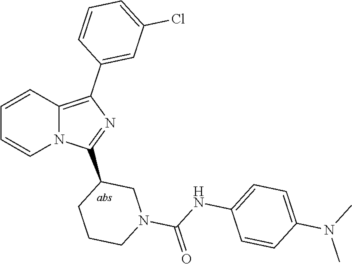 [SMILES: CN(C)c1ccc(NC(=O)N2CCC[C@@H](c3nc(-c4cccc(Cl)c4)c4ccccn34)C2)cc1] | 10-22 |
| 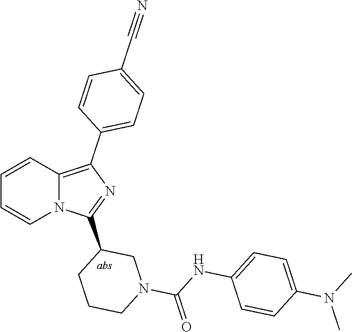 [SMILES: CN(C)c1ccc(NC(=O)N2CCC[C@@H](c3nc(-c4ccc(C#N)cc4)c4ccccn34)C2)cc1] | 10-23 |
| 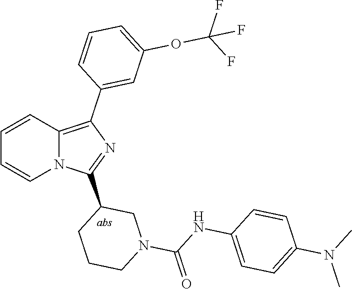 [SMILES: CN(C)c1ccc(NC(=O)N2CCC[C@@H](c3nc(-c4cccc(OC(F)(F)F)c4)c4ccccn34)C2)cc1] | 10-24 |
| 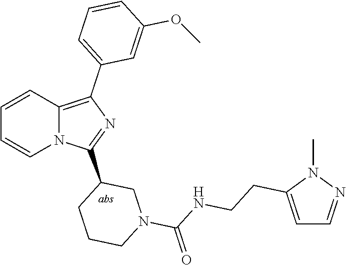 [SMILES: COc1cccc(-c2nc([C@@H]3CCCN(C(=O)NCCc4ccnn4C)C3)n3ccccc23)c1] | 10-25 |
| 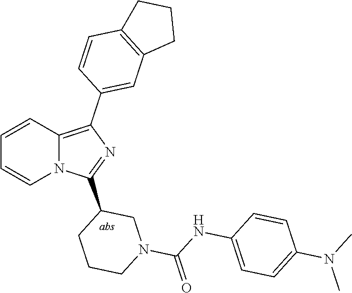 [SMILES: CN(C)c1ccc(NC(=O)N2CCC[C@@H](c3nc(-c4ccc5c(c4)CCC5)c4ccccn34)C2)cc1] | 10-26 |
| 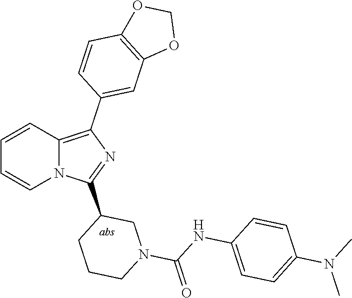 [SMILES: CN(C)c1ccc(NC(=O)N2CCC[C@@H](c3nc(-c4ccc5c(c4)OCO5)c4ccccn34)C2)cc1] | 10-27 |
| 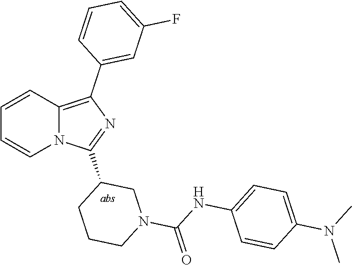 [SMILES: CN(C)c1ccc(NC(=O)N2CCC[C@H](c3nc(-c4cccc(F)c4)c4ccccn34)C2)cc1] | 10-28 |
| 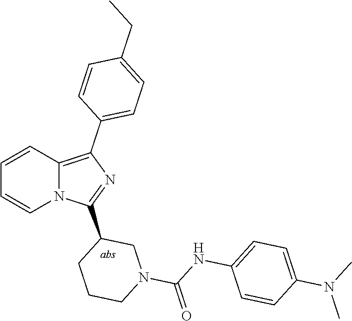 [SMILES: CCc1ccc(-c2nc([C@@H]3CCCN(C(=O)Nc4ccc(N(C)C)cc4)C3)n3ccccc23)cc1] | 10-29 |
| 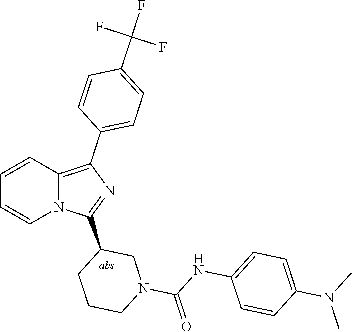 [SMILES: CN(C)c1ccc(NC(=O)N2CCC[C@@H](c3nc(-c4ccc(C(F)(F)F)cc4)c4ccccn34)C2)cc1] | 10-30 |
| 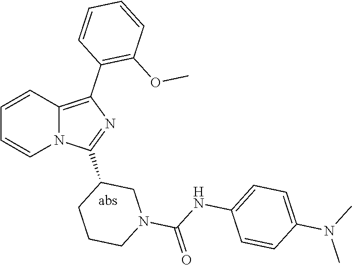 [SMILES: COc1ccccc1-c1nc([C@H]2CCCN(C(=O)Nc3ccc(N(C)C)cc3)C2)n2ccccc12] | 10-31 |
| 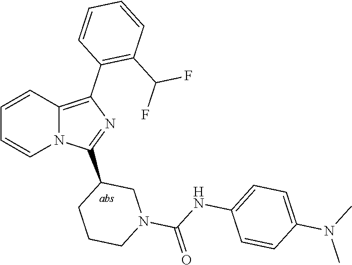 [SMILES: CN(C)c1ccc(NC(=O)N2CCC[C@@H](c3nc(-c4ccccc4C(F)F)c4ccccn34)C2)cc1] | 10-32 |
| 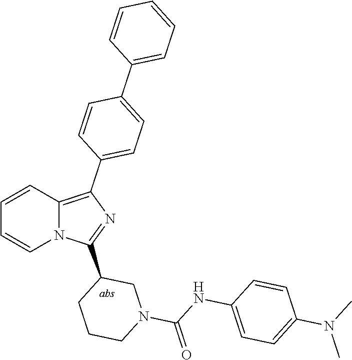 [SMILES: CN(C)c1ccc(NC(=O)N2CCC[C@@H](c3nc(-c4ccc(-c5ccccc5)cc4)c4ccccn34)C2)cc1] | 10-33 |
| 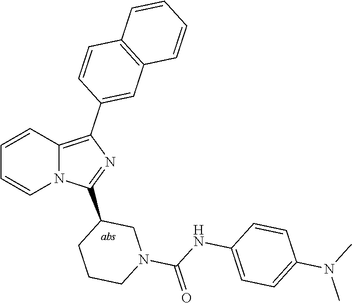 [SMILES: CN(C)c1ccc(NC(=O)N2CCC[C@@H](c3nc(-c4ccc5ccccc5c4)c4ccccn34)C2)cc1] | 10-34 |
| 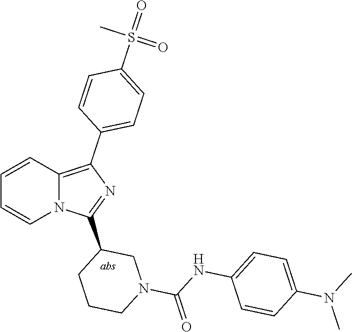 [SMILES: CN(C)c1ccc(NC(=O)N2CCC[C@@H](c3nc(-c4ccc(S(C)(=O)=O)cc4)c4ccccn34)C2)cc1] | 10-35 |
| 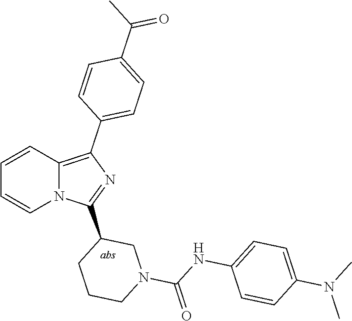 [SMILES: CC(=O)c1ccc(-c2nc([C@@H]3CCCN(C(=O)Nc4ccc(N(C)C)cc4)C3)n3ccccc23)cc1] | 10-36 |
| 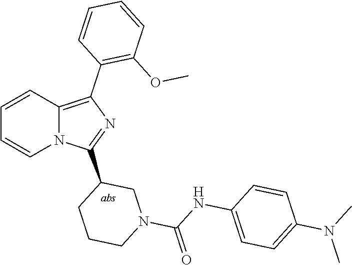 [SMILES: COc1ccccc1-c1nc([C@@H]2CCCN(C(=O)Nc3ccc(N(C)C)cc3)C2)n2ccccc12] | 10-37 |
| 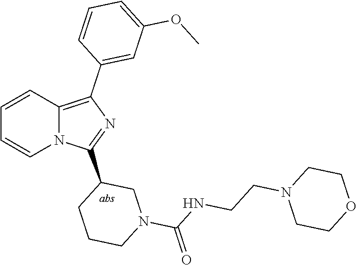 [SMILES: COc1cccc(-c2nc([C@@H]3CCCN(C(=O)NCCN4CCOCC4)C3)n3ccccc23)c1] | 10-38 |
| 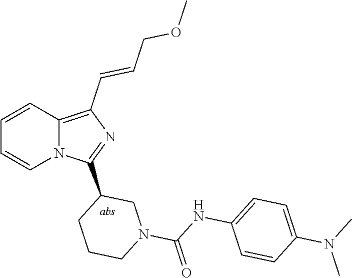 [SMILES: COC/C=C/c1nc([C@@H]2CCCN(C(=O)Nc3ccc(N(C)C)cc3)C2)n2ccccc12] | 10-39 |
| 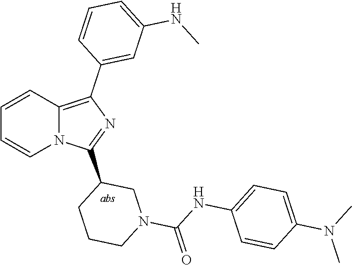 [SMILES: CNc1cccc(-c2nc([C@@H]3CCCN(C(=O)Nc4ccc(N(C)C)cc4)C3)n3ccccc23)c1] | 10-40 |
| 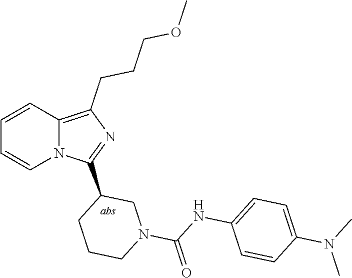 [SMILES: COCCCc1nc([C@@H]2CCCN(C(=O)Nc3ccc(N(C)C)cc3)C2)n2ccccc12] | 10-41 |
| 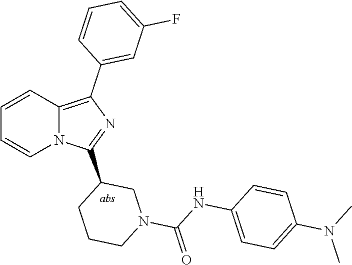 [SMILES: CN(C)c1ccc(NC(=O)N2CCC[C@@H](c3nc(-c4cccc(F)c4)c4ccccn34)C2)cc1] | 10-42 |
| 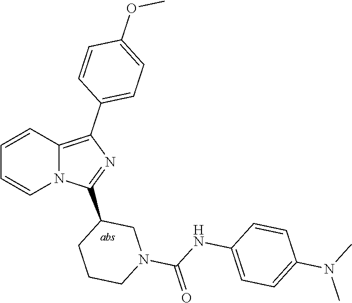 [SMILES: COc1ccc(-c2nc([C@@H]3CCCN(C(=O)Nc4ccc(N(C)C)cc4)C3)n3ccccc23)cc1] | 10-43 |
| 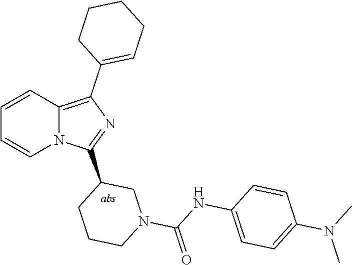 [SMILES: CN(C)c1ccc(NC(=O)N2CCC[C@@H](c3nc(C4=CCCCC4)c4ccccn34)C2)cc1] | 10-44 |
| 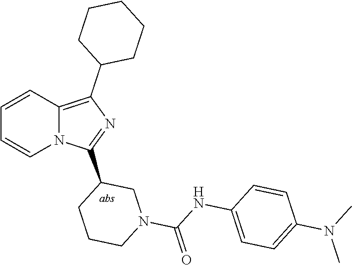 [SMILES: CN(C)c1ccc(NC(=O)N2CCC[C@@H](c3nc(C4CCCCC4)c4ccccn34)C2)cc1] | 10-45 |
| 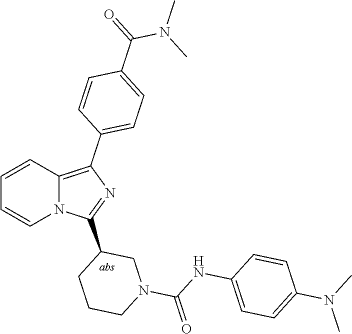 [SMILES: CN(C)C(=O)c1ccc(-c2nc([C@@H]3CCCN(C(=O)Nc4ccc(N(C)C)cc4)C3)n3ccccc23)cc1] | 10-46 |
| 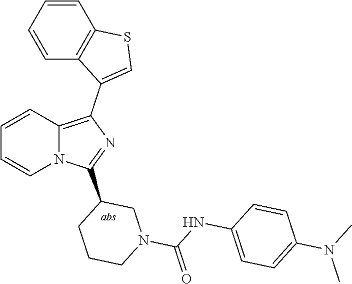 [SMILES: CN(C)c1ccc(NC(=O)N2CCC[C@@H](c3nc(-c4csc5ccccc45)c4ccccn34)C2)cc1] | 10-47 |
| 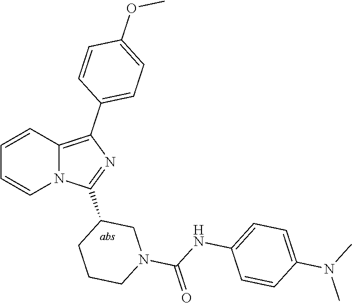 [SMILES: COc1ccc(-c2nc([C@H]3CCCN(C(=O)Nc4ccc(N(C)C)cc4)C3)n3ccccc23)cc1] | 10-48 |
| 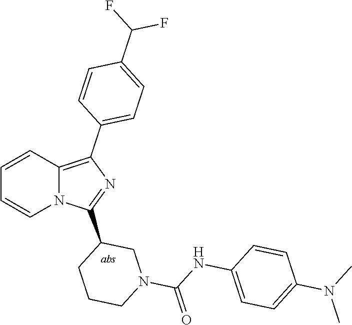 [SMILES: CN(C)c1ccc(NC(=O)N2CCC[C@@H](c3nc(-c4ccc(C(F)F)cc4)c4ccccn34)C2)cc1] | 10-49 |
| 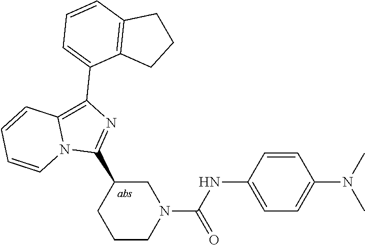 [SMILES: CN(C)c1ccc(NC(=O)N2CCC[C@@H](c3nc(-c4cccc5c4CCC5)c4ccccn34)C2)cc1] | 10-50 |
| 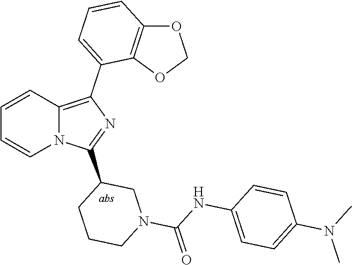 [SMILES: CN(C)c1ccc(NC(=O)N2CCC[C@@H](c3nc(-c4cccc5c4OCO5)c4ccccn34)C2)cc1] | 10-51 |
| 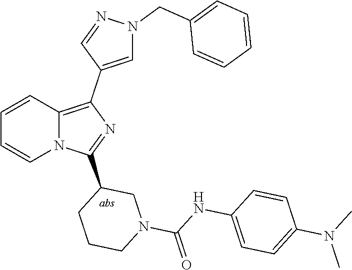 [SMILES: CN(C)c1ccc(NC(=O)N2CCC[C@@H](c3nc(-c4cnn(Cc5ccccc5)c4)c4ccccn34)C2)cc1] | 10-52 |
| 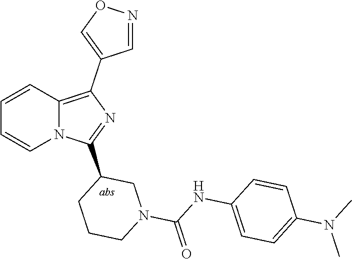 [SMILES: CN(C)c1ccc(NC(=O)N2CCC[C@@H](c3nc(-c4cnoc4)c4ccccn34)C2)cc1] | 10-53 |
| 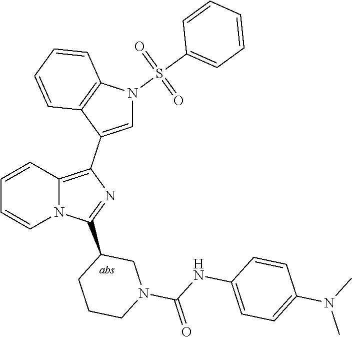 [SMILES: CN(C)c1ccc(NC(=O)N2CCC[C@@H](c3nc(-c4cn(S(=O)(=O)c5ccccc5)c5ccccc45)c4ccccn34)C2)cc1] | 10-54 |
| 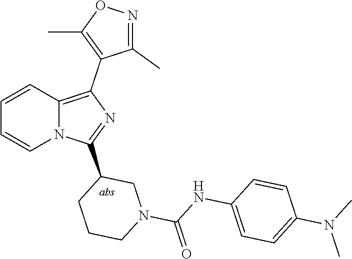 [SMILES: Cc1noc(C)c1-c1nc([C@@H]2CCCN(C(=O)Nc3ccc(N(C)C)cc3)C2)n2ccccc12] | 10-55 |
| 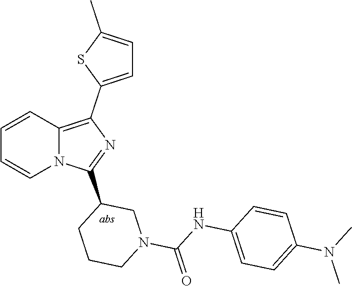 [SMILES: Cc1ccc(-c2nc([C@@H]3CCCN(C(=O)Nc4ccc(N(C)C)cc4)C3)n3ccccc23)s1] | 10-56 |
| 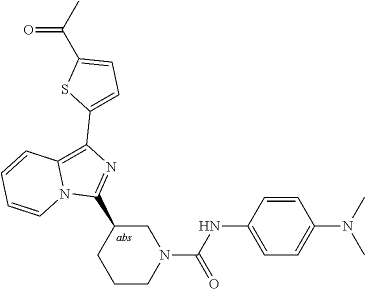 [SMILES: CC(=O)c1ccc(-c2nc([C@@H]3CCCN(C(=O)Nc4ccc(N(C)C)cc4)C3)n3ccccc23)s1] | 10-57 |
| 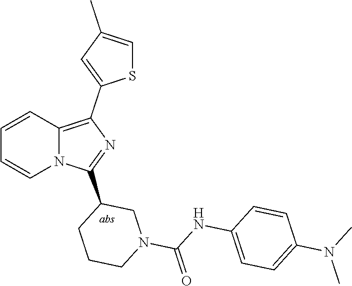 [SMILES: Cc1csc(-c2nc([C@@H]3CCCN(C(=O)Nc4ccc(N(C)C)cc4)C3)n3ccccc23)c1] | 10-58 |
| 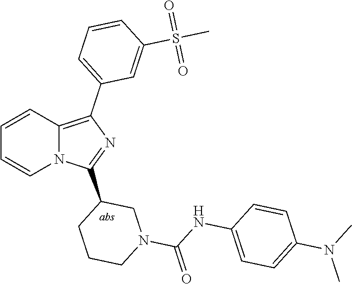 [SMILES: CN(C)c1ccc(NC(=O)N2CCC[C@@H](c3nc(-c4cccc(S(C)(=O)=O)c4)c4ccccn34)C2)cc1] | 10-59 |
| 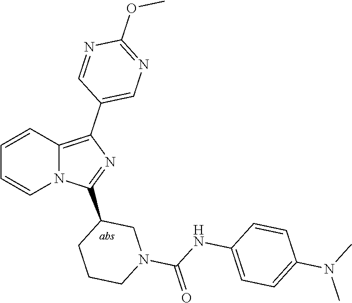 [SMILES: COc1ncc(-c2nc([C@@H]3CCCN(C(=O)Nc4ccc(N(C)C)cc4)C3)n3ccccc23)cn1] | 10-60 |
| 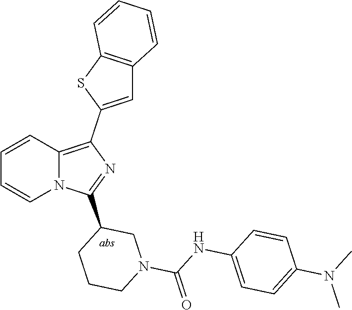 [SMILES: CN(C)c1ccc(NC(=O)N2CCC[C@@H](c3nc(-c4cc5ccccc5s4)c4ccccn34)C2)cc1] | 10-61 |
| 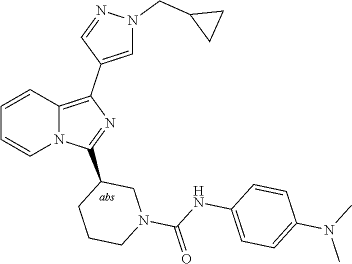 [SMILES: CN(C)c1ccc(NC(=O)N2CCC[C@@H](c3nc(-c4cnn(CC5CC5)c4)c4ccccn34)C2)cc1] | 10-62 |
| 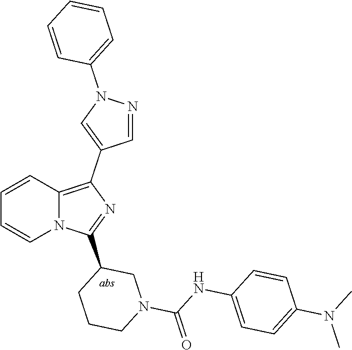 [SMILES: CN(C)c1ccc(NC(=O)N2CCC[C@@H](c3nc(-c4cnn(-c5ccccc5)c4)c4ccccn34)C2)cc1] | 10-63 |
| 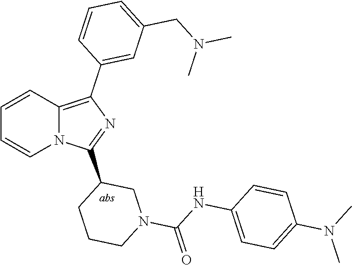 [SMILES: CN(C)Cc1cccc(-c2nc([C@@H]3CCCN(C(=O)Nc4ccc(N(C)C)cc4)C3)n3ccccc23)c1] | 10-64 |
| 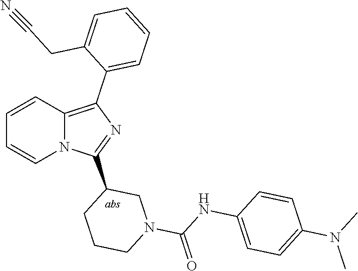 [SMILES: CN(C)c1ccc(NC(=O)N2CCC[C@@H](c3nc(-c4ccccc4CC#N)c4ccccn34)C2)cc1] | 10-65 |
| 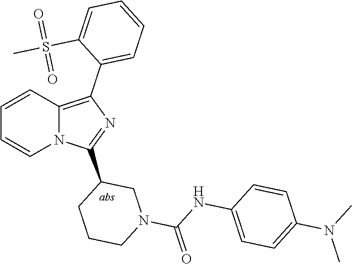 [SMILES: CN(C)c1ccc(NC(=O)N2CCC[C@@H](c3nc(-c4ccccc4S(C)(=O)=O)c4ccccn34)C2)cc1] | 10-66 |
| 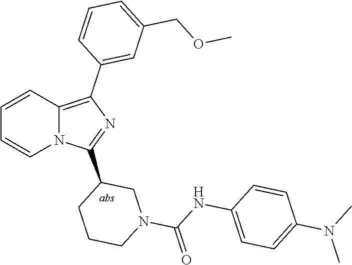 [SMILES: COCc1cccc(-c2nc([C@@H]3CCCN(C(=O)Nc4ccc(N(C)C)cc4)C3)n3ccccc23)c1] | 10-67 |
| 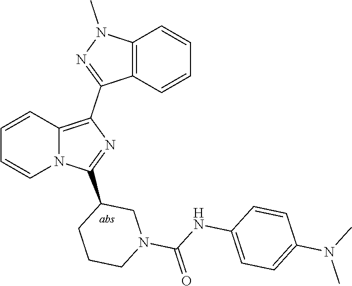 [SMILES: CN(C)c1ccc(NC(=O)N2CCC[C@@H](c3nc(-c4nn(C)c5ccccc45)c4ccccn34)C2)cc1] | 10-68 |
| 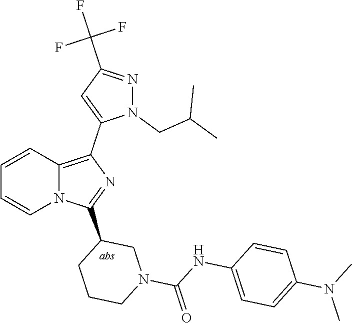 [SMILES: CC(C)Cn1nc(C(F)(F)F)cc1-c1nc([C@@H]2CCCN(C(=O)Nc3ccc(N(C)C)cc3)C2)n2ccccc12] | 10-69 |
| 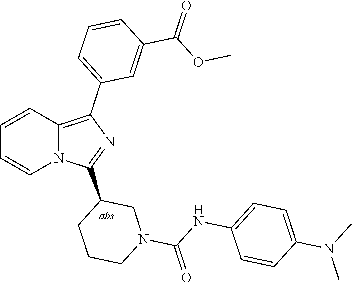 [SMILES: COC(=O)c1cccc(-c2nc([C@@H]3CCCN(C(=O)Nc4ccc(N(C)C)cc4)C3)n3ccccc23)c1] | 10-70 |
| 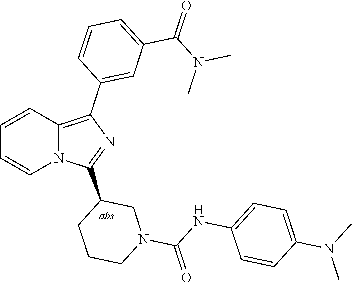 [SMILES: CN(C)C(=O)c1cccc(-c2nc([C@@H]3CCCN(C(=O)Nc4ccc(N(C)C)cc4)C3)n3ccccc23)c1] | 10-71 |
| 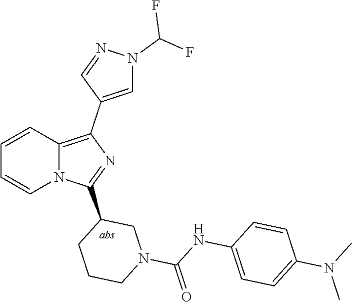 [SMILES: CN(C)c1ccc(NC(=O)N2CCC[C@@H](c3nc(-c4cnn(C(F)F)c4)c4ccccn34)C2)cc1] | 10-72 |
| 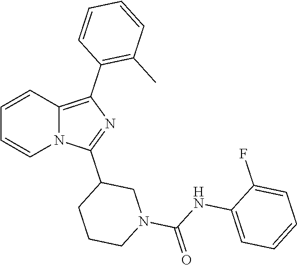 [SMILES: Cc1ccccc1-c1nc(C2CCCN(C(=O)Nc3ccccc3F)C2)n2ccccc12] | 11-1 |
| 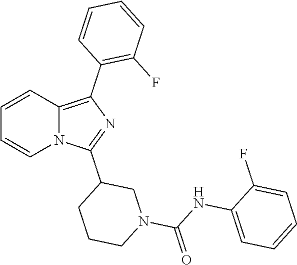 [SMILES: O=C(Nc1ccccc1F)N1CCCC(c2nc(-c3ccccc3F)c3ccccn23)C1] | 11-2 |
| 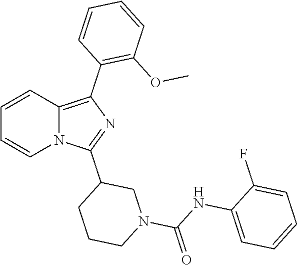 [SMILES: COc1ccccc1-c1nc(C2CCCN(C(=O)Nc3ccccc3F)C2)n2ccccc12] | 11-3 |
| 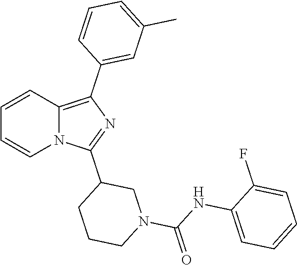 [SMILES: Cc1cccc(-c2nc(C3CCCN(C(=O)Nc4ccccc4F)C3)n3ccccc23)c1] | 11-4 |
| 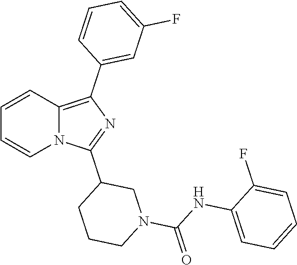 [SMILES: O=C(Nc1ccccc1F)N1CCCC(c2nc(-c3cccc(F)c3)c3ccccn23)C1] | 11-5 |
| 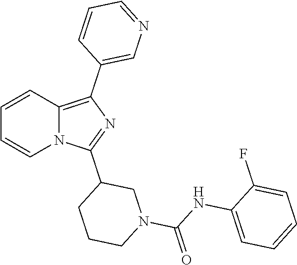 [SMILES: O=C(Nc1ccccc1F)N1CCCC(c2nc(-c3cccnc3)c3ccccn23)C1] | 11-6 |
| 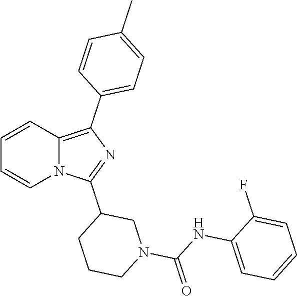 [SMILES: Cc1ccc(-c2nc(C3CCCN(C(=O)Nc4ccccc4F)C3)n3ccccc23)cc1] | 11-7 |
| 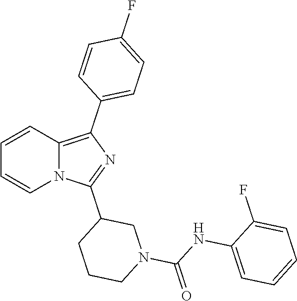 [SMILES: O=C(Nc1ccccc1F)N1CCCC(c2nc(-c3ccc(F)cc3)c3ccccn23)C1] | 11-8 |
| 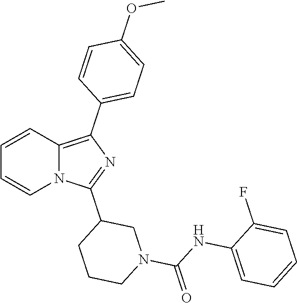 [SMILES: COc1ccc(-c2nc(C3CCCN(C(=O)Nc4ccccc4F)C3)n3ccccc23)cc1] | 11-9 |
| 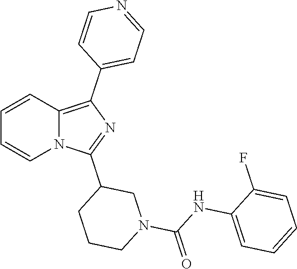 [SMILES: O=C(Nc1ccccc1F)N1CCCC(c2nc(-c3ccncc3)c3ccccn23)C1] | 11-10 |
| 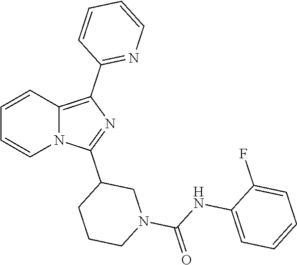 [SMILES: O=C(Nc1ccccc1F)N1CCCC(c2nc(-c3ccccn3)c3ccccn23)C1] | 11-11 |
| 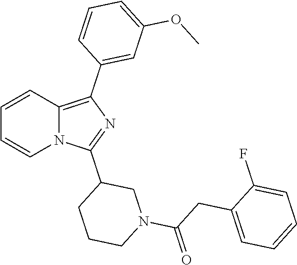 [SMILES: COc1cccc(-c2nc(C3CCCN(C(=O)Cc4ccccc4F)C3)n3ccccc23)c1] | 12-1 |
| 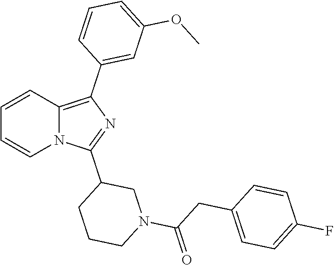 [SMILES: COc1cccc(-c2nc(C3CCCN(C(=O)Cc4ccc(F)cc4)C3)n3ccccc23)c1] | 12-2 |
| 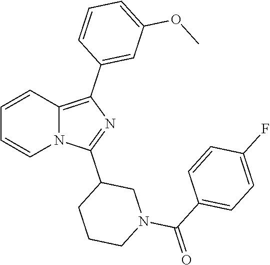 [SMILES: COc1cccc(-c2nc(C3CCCN(C(=O)c4ccc(F)cc4)C3)n3ccccc23)c1] | 12-3 |
| 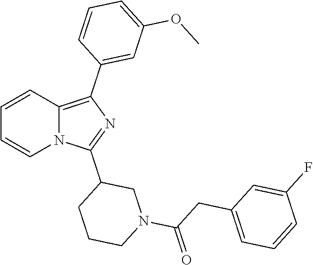 [SMILES: COc1cccc(-c2nc(C3CCCN(C(=O)Cc4cccc(F)c4)C3)n3ccccc23)c1] | 12-4 |
| 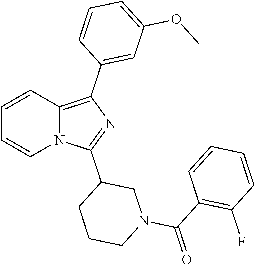 [SMILES: COc1cccc(-c2nc(C3CCCN(C(=O)c4ccccc4F)C3)n3ccccc23)c1] | 12-5 |
| 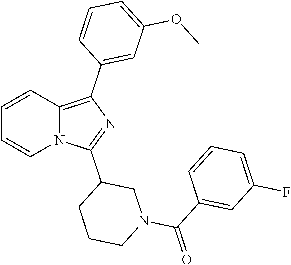 [SMILES: COc1cccc(-c2nc(C3CCCN(C(=O)c4cccc(F)c4)C3)n3ccccc23)c1] | 12-6 |
| 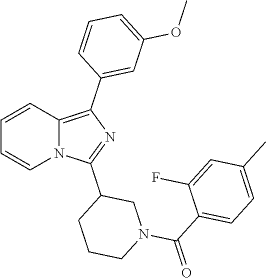 [SMILES: COc1cccc(-c2nc(C3CCCN(C(=O)c4ccc(C)cc4F)C3)n3ccccc23)c1] | 12-7 |
| 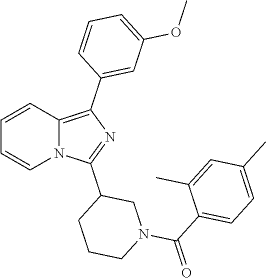 [SMILES: COc1cccc(-c2nc(C3CCCN(C(=O)c4ccc(C)cc4C)C3)n3ccccc23)c1] | 12-8 |
| 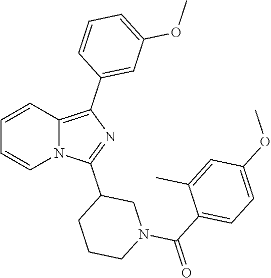 [SMILES: COc1cccc(-c2nc(C3CCCN(C(=O)c4ccc(OC)cc4C)C3)n3ccccc23)c1] | 12-9 |
| 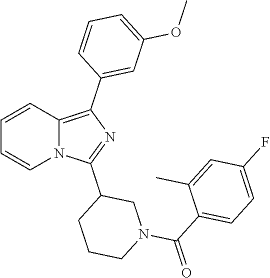 [SMILES: COc1cccc(-c2nc(C3CCCN(C(=O)c4ccc(F)cc4C)C3)n3ccccc23)c1] | 12-10 |
| 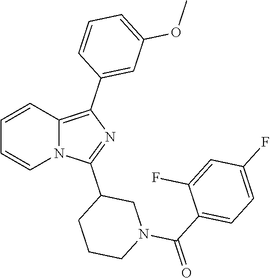 [SMILES: COc1cccc(-c2nc(C3CCCN(C(=O)c4ccc(F)cc4F)C3)n3ccccc23)c1] | 12-11 |
| 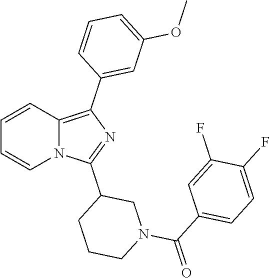 [SMILES: COc1cccc(-c2nc(C3CCCN(C(=O)c4ccc(F)c(F)c4)C3)n3ccccc23)c1] | 12-12 |
| 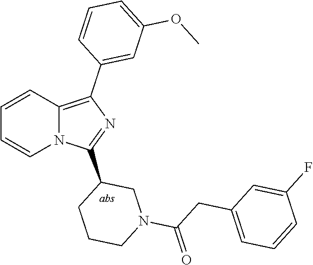 [SMILES: COc1cccc(-c2nc([C@@H]3CCCN(C(=O)Cc4cccc(F)c4)C3)n3ccccc23)c1] | 12-13 |
| 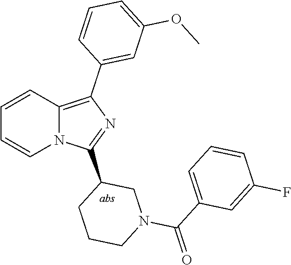 [SMILES: COc1cccc(-c2nc([C@@H]3CCCN(C(=O)c4cccc(F)c4)C3)n3ccccc23)c1] | 12-14 |
| 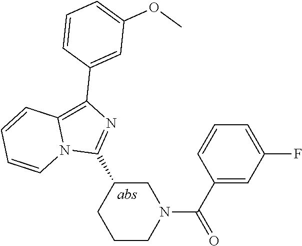 [SMILES: COc1cccc(-c2nc([C@H]3CCCN(C(=O)c4cccc(F)c4)C3)n3ccccc23)c1] | 12-15 |
| 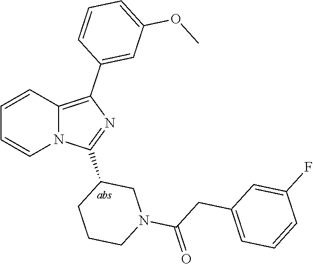 [SMILES: COc1cccc(-c2nc([C@H]3CCCN(C(=O)Cc4cccc(F)c4)C3)n3ccccc23)c1] | 12-16 |
| 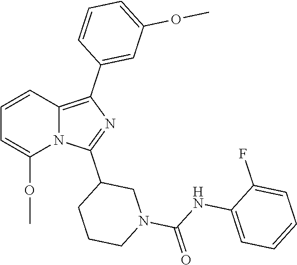 [SMILES: COc1cccc(-c2nc(C3CCCN(C(=O)Nc4ccccc4F)C3)n3c(OC)cccc23)c1] | 13-1 |
| 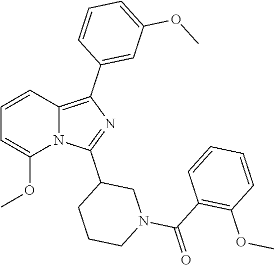 [SMILES: COc1cccc(-c2nc(C3CCCN(C(=O)c4ccccc4OC)C3)n3c(OC)cccc23)c1] | 14-1 |
| 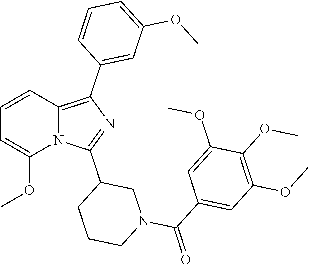 [SMILES: COc1cccc(-c2nc(C3CCCN(C(=O)c4cc(OC)c(OC)c(OC)c4)C3)n3c(OC)cccc23)c1] | 14-2 |
| 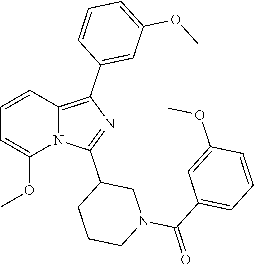 [SMILES: COc1cccc(C(=O)N2CCCC(c3nc(-c4cccc(OC)c4)c4cccc(OC)n34)C2)c1] | 14-3 |
| 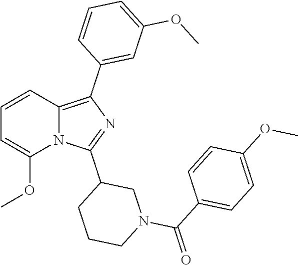 [SMILES: COc1ccc(C(=O)N2CCCC(c3nc(-c4cccc(OC)c4)c4cccc(OC)n34)C2)cc1] | 14-4 |
| 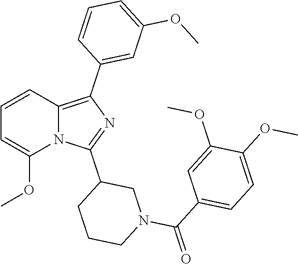 [SMILES: COc1cccc(-c2nc(C3CCCN(C(=O)c4ccc(OC)c(OC)c4)C3)n3c(OC)cccc23)c1] | 14-5 |
| 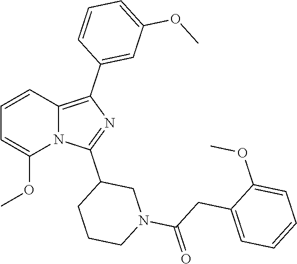 [SMILES: COc1cccc(-c2nc(C3CCCN(C(=O)Cc4ccccc4OC)C3)n3c(OC)cccc23)c1] | 15-1 |
| 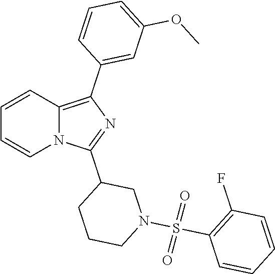 [SMILES: COc1cccc(-c2nc(C3CCCN(S(=O)(=O)c4ccccc4F)C3)n3ccccc23)c1] | 16-1 |
| 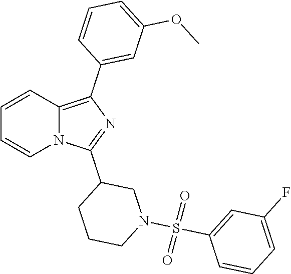 [SMILES: COc1cccc(-c2nc(C3CCCN(S(=O)(=O)c4cccc(F)c4)C3)n3ccccc23)c1] | 16-2 |
| 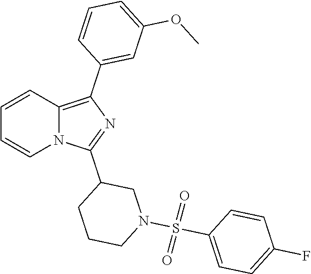 [SMILES: COc1cccc(-c2nc(C3CCCN(S(=O)(=O)c4ccc(F)cc4)C3)n3ccccc23)c1] | 16-3 |
| 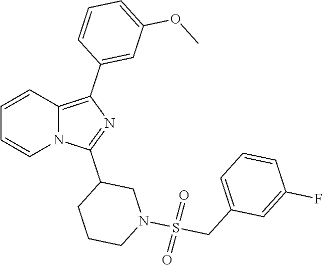 [SMILES: COc1cccc(-c2nc(C3CCCN(S(=O)(=O)Cc4cccc(F)c4)C3)n3ccccc23)c1] | 16-4 |
| 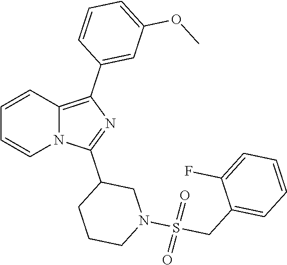 [SMILES: COc1cccc(-c2nc(C3CCCN(S(=O)(=O)Cc4ccccc4F)C3)n3ccccc23)c1] | 16-5 |
| 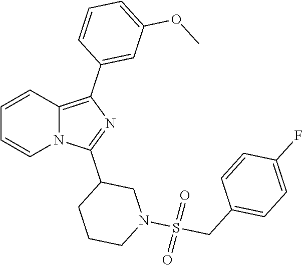 [SMILES: COc1cccc(-c2nc(C3CCCN(S(=O)(=O)Cc4ccc(F)cc4)C3)n3ccccc23)c1] | 16-6 |
| 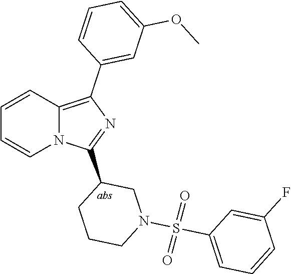 [SMILES: COc1cccc(-c2nc([C@@H]3CCCN(S(=O)(=O)c4cccc(F)c4)C3)n3ccccc23)c1] | 16-7 |
| 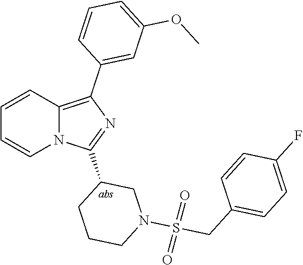 [SMILES: COc1cccc(-c2nc([C@H]3CCCN(S(=O)(=O)Cc4ccc(F)cc4)C3)n3ccccc23)c1] | 16-8 |
| 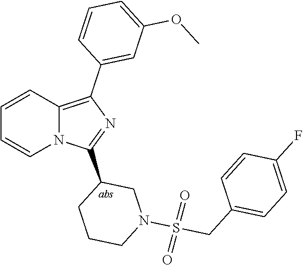 [SMILES: COc1cccc(-c2nc([C@@H]3CCCN(S(=O)(=O)Cc4ccc(F)cc4)C3)n3ccccc23)c1] | 16-9 |
| 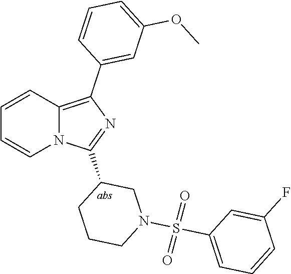 [SMILES: COc1cccc(-c2nc([C@H]3CCCN(S(=O)(=O)c4cccc(F)c4)C3)n3ccccc23)c1] | 16-10 |
| 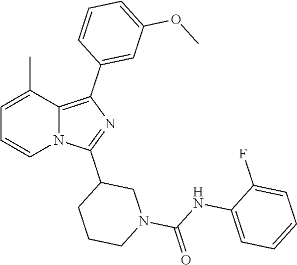 [SMILES: COc1cccc(-c2nc(C3CCCN(C(=O)Nc4ccccc4F)C3)n3cccc(C)c23)c1] | 17-1 |
| 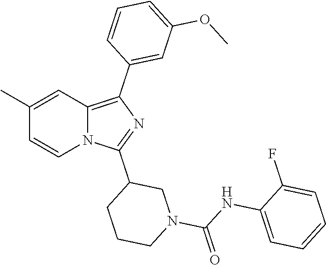 [SMILES: COc1cccc(-c2nc(C3CCCN(C(=O)Nc4ccccc4F)C3)n3ccc(C)cc23)c1] | 17-2 |
| 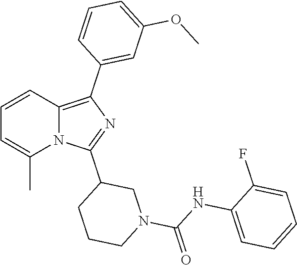 [SMILES: COc1cccc(-c2nc(C3CCCN(C(=O)Nc4ccccc4F)C3)n3c(C)cccc23)c1] | 17-3 |
| 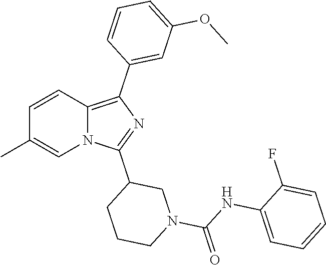 [SMILES: COc1cccc(-c2nc(C3CCCN(C(=O)Nc4ccccc4F)C3)n3cc(C)ccc23)c1] | 18-1 |
| 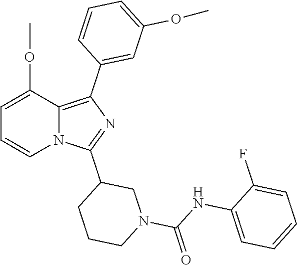 [SMILES: COc1cccc(-c2nc(C3CCCN(C(=O)Nc4ccccc4F)C3)n3cccc(OC)c23)c1] | 19-1 |
| 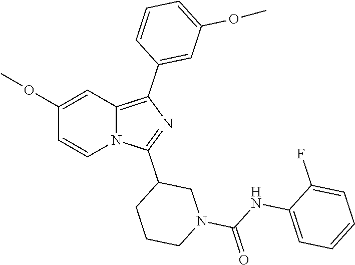 [SMILES: COc1cccc(-c2nc(C3CCCN(C(=O)Nc4ccccc4F)C3)n3ccc(OC)cc23)c1] | 19-2 |
| 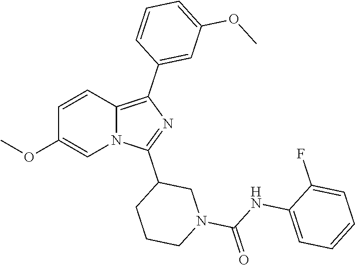 [SMILES: COc1cccc(-c2nc(C3CCCN(C(=O)Nc4ccccc4F)C3)n3cc(OC)ccc23)c1] | 19-3 |
| 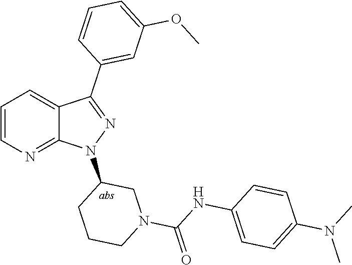 [SMILES: COc1cccc(-c2nn([C@@H]3CCCN(C(=O)Nc4ccc(N(C)C)cc4)C3)c3ncccc23)c1] | 20-1 |
| 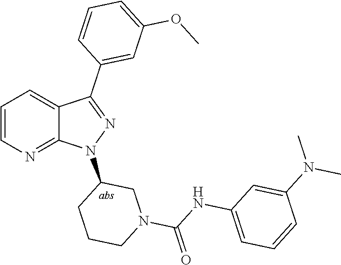 [SMILES: COc1cccc(-c2nn([C@@H]3CCCN(C(=O)Nc4cccc(N(C)C)c4)C3)c3ncccc23)c1] | 20-2 |
| 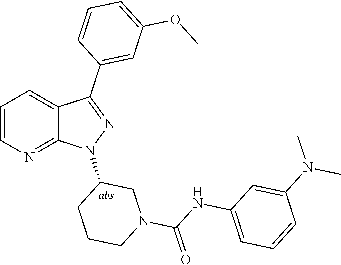 [SMILES: COc1cccc(-c2nn([C@H]3CCCN(C(=O)Nc4cccc(N(C)C)c4)C3)c3ncccc23)c1] | 20-3 |
| 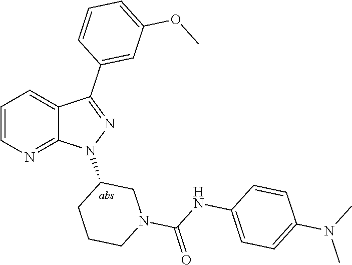 [SMILES: COc1cccc(-c2nn([C@H]3CCCN(C(=O)Nc4ccc(N(C)C)cc4)C3)c3ncccc23)c1] | 20-4 |
| 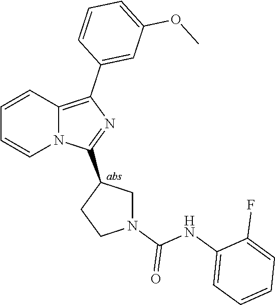 [SMILES: COc1cccc(-c2nc([C@@H]3CCN(C(=O)Nc4ccccc4F)C3)n3ccccc23)c1] | 21-1 |
| 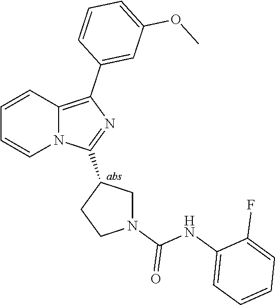 [SMILES: COc1cccc(-c2nc([C@H]3CCN(C(=O)Nc4ccccc4F)C3)n3ccccc23)c1] | 21-2 |
| 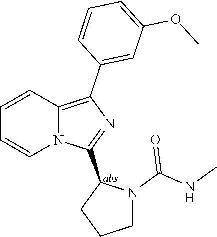 [SMILES: CNC(=O)N1CCC[C@H]1c1nc(-c2cccc(OC)c2)c2ccccn12] | 21-3 |
| 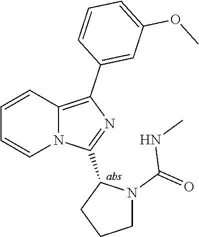 [SMILES: CNC(=O)N1CCC[C@@H]1c1nc(-c2cccc(OC)c2)c2ccccn12] | 21-4 |
| 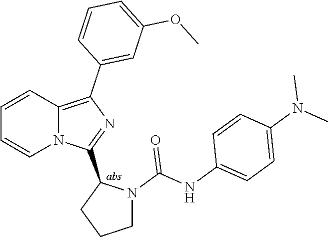 [SMILES: COc1cccc(-c2nc([C@@H]3CCCN3C(=O)Nc3ccc(N(C)C)cc3)n3ccccc23)c1] | 21-5 |
| 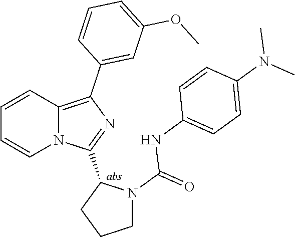 [SMILES: COc1cccc(-c2nc([C@H]3CCCN3C(=O)Nc3ccc(N(C)C)cc3)n3ccccc23)c1] | 21-6 |
| 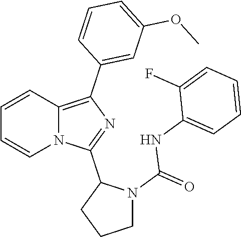 [SMILES: COc1cccc(-c2nc(C3CCCN3C(=O)Nc3ccccc3F)n3ccccc23)c1] | 22-1 |
| 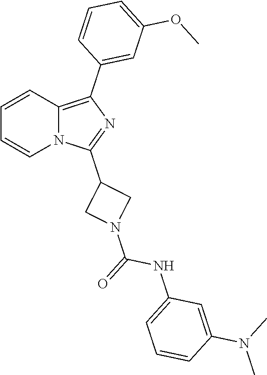 [SMILES: COc1cccc(-c2nc(C3CN(C(=O)Nc4cccc(N(C)C)c4)C3)n3ccccc23)c1] | 23-1 |
| 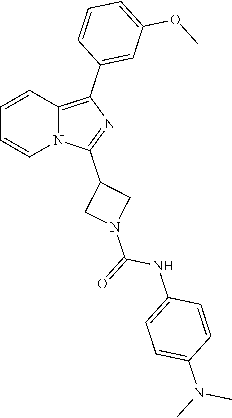 [SMILES: COc1cccc(-c2nc(C3CN(C(=O)Nc4ccc(N(C)C)cc4)C3)n3ccccc23)c1] | 23-2 |
| 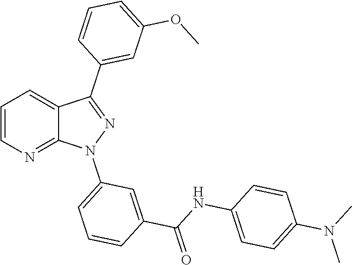 [SMILES: COc1cccc(-c2nn(-c3cccc(C(=O)Nc4ccc(N(C)C)cc4)c3)c3ncccc23)c1] | 24-1 |
| 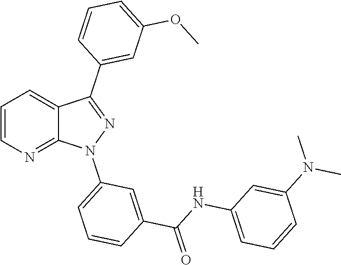 [SMILES: COc1cccc(-c2nn(-c3cccc(C(=O)Nc4cccc(N(C)C)c4)c3)c3ncccc23)c1] | 24-2 |
| 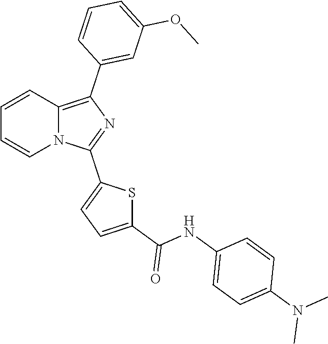 [SMILES: COc1cccc(-c2nc(-c3ccc(C(=O)Nc4ccc(N(C)C)cc4)s3)n3ccccc23)c1] | 25-1 |
| 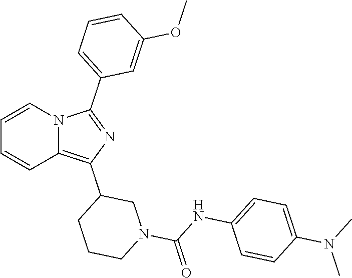 [SMILES: COc1cccc(-c2nc(C3CCCN(C(=O)Nc4ccc(N(C)C)cc4)C3)c3ccccn23)c1] | 26-1 |

### INDICATIONS

### Pain

In one embodiment, the malcondition for which modulation of MRGPRD is medically indicated, the pain associated condition is: Acute Pain, Advanced Prostate Cancer, AIDS-Related Pain, Ankylosing Spondylitis, Arachnoiditis, Arthritis, Arthrofibrosis, Ataxic Cerebral Palsy, Autoimmune Atrophic Gastritis, Avascular Necrosis, Back Pain, Behcet's Disease (Syndrome), Burning Mouth Syndrome, Bursitis, Cancer Pain, Carpal Tunnel, Cauda Equina Syndrome, Central Pain Syndrome, Cerebral Palsy, Cervical Stenosis, Charcot-Marie-Tooth (CMT) Disease, Chronic Fatigue Syndrome (CFS), Chronic Functional Abdominal Pain (CFAP), Chronic Pain, Chronic Pancreatitis, Chronic Pelvic Pain Syndrome, Collapsed Lung (Pneumothorax), Complex Regional Pain Syndrome (RSD), Constipation, Corneal Neuropathic Pain, Crohn's Disease, Degenerative Disc Disease, Dental Pain, Dercum's Disease, Dermatomyositis, Diabetic Peripheral Neuropathy (DPN), Dry Eye Syndrome, Dystonia, Ehlers-Danlos Syndrome (EDS), Endometriosis, Eosinophilia-Myalgia Syndrome (EMS), Erythromelalgia, Fibromyalgia, Gout, Headaches, Herniated disc, Hydrocephalus, Inflammatory Bowel Disease (IBD), Intercostal Neuraligia, Interstitial Cystitis, Irritable Bowel syndrome (IBS), Juvenile Dermatositis (Dermatomyositis), Knee Injury, Leg Pain, Loin Pain-Haematuria Syndrome, Lupus, Lyme Disease, Medullary Sponge Kidney (MSK), Meralgia Paresthetica, Mesothelioma, Migraine, Musculoskeletal pain, Myofascial Pain, Myositis, Neck Pain, Neuropathic Pain, Occipital Neuralgia, ocular Itch, Osteoarthritis, Paget's Disease, Parsonage Turner Syndrome, Pelvic Pain, Periodontitis Pain, Peripheral Neuropathy, Phantom Limb Pain, Pinched Nerve, Polycystic Kidney Disease, Polymyalgia Rhuematica, Polymyositis, Porphyria, Post Herniorraphy Pain Syndrome, Post Mastectomy, Postoperative Pain, Pain Syndrome, Post Stroke Pain, Post Thorocotomy Pain Syndrome, Postherpetic Neuralgia (Shingles), Post-Polio Syndrome, Primary Lateral Sclerosis, Psoriatic Arthritis, Pudendal Neuralgia, Radiculopathy, Raynaud's Disease, Rheumatoid Arthritis (RA), Sacroiliac Joint Dysfunction, Sarcoidosi, Scheuemann's Kyphosis Disease, Sciatica, Scoliosis, Shingles (Herpes Zoster), Sjogren's Syndrome, Spasmodic Torticollis, Sphincter of Oddi Dysfunction, Spinal Cerebellum Ataxia (SCA Ataxia), Spinal Cord Injury, Spinal Stenosis, Syringomyelia, Tarlov Cysts, Transverse Myelitis, Trigeminal Neuralgia, Neuropathic Pain, Ulcerative Colitis, Vascular Pain or Vulvodynia.

### Skin

In another embodiment, the itch associated condition is: chronic itch; contact dermatitis; Allergic blepharitis; Anemia; Atopic dermatitis; Bullous pemphigoid; Candidiasis; Chicken pox; end-stage renal failure; hemodialysis; Chronic urticaria; Contact dermatitis, Atopic Dermatitis; Dermatitis herpetiformis; Diabetes; Drug allergy, Dry skin; Dyshidrotic dermatitis; Ectopic eczema; Eosinophilic fasciitis; Epidermolysis bullosa; Erythrasma; Food allergy; Folliculitis; Fungal skin infection; Hemorrhoids; Herpes; HIV infection; Hodgkin's disease; Hyperthyroidism; Iodinated contrast dye allergy; Iron deficiency anemia; Kidney disease; Leukemia, porphyrias; Lymphoma; Malignancy; Mastocystosis; Multiple myeloma; Neurodermatitis; Onchocerciasis; Paget's disease; Pediculosis; Polycythemia rubra vera; Prurigo nodularis; Lichen Planus; Lichen Sclerosis; Pruritus ani; Pseudorabies; Psoriasis; Rectal prolapse; Sarcoidosis granulomas; Scabies; Schistosomiasis; Scleroderma, Severe stress, Stasia dermatitis; Swimmer's itch; Thyroid disease; Tinea cruris; Rosacea; Cutaneous amyloidosis; Scleroderma; Acne; wound healing; burn healing; ocular itch; or Urticaria.

In a specific embodiment, the itch associated condition is urticaria, pruritus, atopic dermatitis, dry skin, psoriasis, contact dermatitis, or eczema.

### Ocular

In another embodiment, the ocular associated condition is: dry eye syndrome/keratoconjunctivitis sicca and related conditions, including xeropthalmia, meibomian gland dysfunction and lacrimal gland dysfunction; dry eye associated with other medical conditions including dacryoadenitis, dacryocystitis, allergic conjunctivitis, blepharitis, rheumatoid arthritis, systemic lupus erythematous, scleroderma, Sjogren's syndrome, Stevens-Johnson syndrome, sarcoidosis, sympathetic opthalmia, diabetic retinopathy, parasitic eye infections, thyroid disorders, and vitamin A deficiency; dry eye associated with medications such as antihistamines, decongestants, anti-depressants, tranquilizers, diuretics, hormone replacement, oral contraceptives, antihypertensives, isotretonin treatments for acne, and anticholinergic drugs; and dry eye associated with eye surgery including laser eye surgery, glaucoma surgery, corneal transplantation, and cataract removal surgery.

### Cardiovascular and Renal

In another embodiment, cardiovascular and renal diseases associated condition is: peripheral vascular disease, cerebrovascular disease, coronary artery disease, cardiac hypertrophy, cardiac fibrosis, cardiovascular hypertension, renovascular hypertension, renal fibrosis, renal disease, nephritis, atherosclerosis, coronary atherosclerotic heart disease, acute myocardial infarction, stroke, thrombosis, coronary atherothrombosis, pulmonary embolism, myocardial ischemia, carotid stenosis, vertebral stenosis, intracranial stenosis, and aneurysms as well as treatment of cardiac dysfunction induced by sepsis, rheumatic fever, or other acute or chronic disorders that influence cardiovascular and renal function such as diabetes.

### Immune/GI

In another embodiment, the chronic inflammatory and autoimmune associated condition include: chronic pulmonary allergy, asthma, chronic bronchitis, atherosclerosis, Graves' disease, Hashimoto's thyroiditis, chronic inflammatory demyelinating polyneuropathy, ankylosing spondylitis, sacroiliitis, steatohepatitis, scleroderma, systemic sclerosis, diabetes, ulcerative colitis, Crohn's disease, inflammatory bowel disease, systemic lupus erythematous, alopecia areata, temporal arteritis, chronic peptic ulcer, polymyalgia rheumatica, periodontitis, sinusitis, rhinitis, pancreatitis, nephritis, Sjogren's syndrome, dermatomyositis, polymyositis, inclusion body myositis, autoimmune necrotizing myopathy, idiopathic inflammatory myopathies, multiple sclerosis, rheumatoid arthritis, and vasculitis.

As used herein, the term “autoimmune disorder”, or “inflammatory disorder” means a disease or disorder arising from and/or directed against an individual's own tissues or organs, or a co-segregate or manifestation thereof, or resulting condition therefrom. Typically, various clinical and laboratory markers of autoimmune diseases may exist including, but not limited to, hypergammaglobulinemia, high levels of autoantibodies, antigen-antibody complex deposits in tissues, clinical benefit from corticosteroid or immunosuppressive treatments, and lymphoid cell aggregates in affected tissues. Thus, in one embodiment, the method of present invention is provided to treat an autoimmune disorder, such as chronic inflammation, mast cell activation syndrome, Multiple Sclerosis, Steven Johnson's Syndrome, Toxic Epidermal Necrolysis, appendicitis, bursitis, cutaneous lupus, colitis, cystitis, dermatitis, phlebitis, reflex sympathetic dystrophy/complex regional pain syndrome (rsd/crps), rhinitis, tendonitis, tonsillitis, acne vulgaris, sinusitis, rosacea, psoriasis, graft-versus-host disease, reactive airway disorder, asthma, airway infection, allergic rhinitis, autoinflammatory disease, celiac disease, chronic prostatitis, diverticulitis, glomerulonephritis, hidradenitis suppurativa, hypersensitivities, intestinal disorder, epithelial intestinal disorder, inflammatory bowel disease, irritable bowel syndrome, Crohn's Disease, ulcerative colitis, lupus erythematous, interstitial cystitis, otitis, pelvic inflammatory disease, endometrial pain, reperfusion injury, rheumatic fever, rheumatoid arthritis, sarcoidosis, transplant rejection, psoriasis, lung inflammation, chronic obstructive pulmonary disease, permanent sputum eosiniophilia, eosinophilic leukemia, eosinophilic esophagitis, eosinophilic gastritis, mast cell gastrointestinal disease, hypereosinophilic syndrome, aspirin-exacerbated respiratory disease, nasal polyposis, chronic rhinosinusitis, antibody-dependent cell-mediated cytotoxicity, neurofibromatosis, swannamatoisis, tubulointerstitial nephritis, glomerulonephritis, diabetic nephropathy, allograft rejection, amyloidosis, renovascular ischemia, reflux nephropathy, polycystic kidney disease, liver fibrosis/cirrhosis, autoimmune liver disease, Biliary atresia, acute and chronic Hepatitis B and C virus, Liver tumors and cancer, Lung tumors and cancer, Alcoholic liver disease, Polycystic liver disease, Liver cholangiocarcinoma, neuromyelitis optica spectum disorder, cardiovascular disease, and vasculitis.

### Cognitive Indications

In another embodiment, cognitive impairment associated condition include neurodegenerative diseases including Parkinson's disease, Alzheimer's disease, Huntington's disease, Lewy body dementia, frontotemporal dementia, progressive supranuclear palsy, corticobasal syndrome, frontotemporal lobar degeneration, amyotrophic lateral sclerosis and multiple sclerosis, as well as age-induced cognitive impairment, vascular cognitive impairment and post-stroke cognitive impairment.

### EXAMPLES

All reagents, for which the synthesis is not described in the experimental part, are either commercially available, or are known compounds or may be formed from known compounds by known methods by a person skilled in the art. The compounds and intermediates produced according to the methods of the invention may require purification. Purification of organic compounds is well known to a person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using purpose-made or prepacked silica gel cartridges and eluents such as gradients of solvents such as heptane, ether, ethyl acetate, acetonitrile, ethanol and the like. In some cases, the compounds may be purified by preparative HPLC using methods as described.

Purification methods as described herein may provide compounds of the present invention which possess a sufficiently basic or acidic functionality in the form of a salt, such as, in the case of a compound of the present invention which is sufficiently basic, a trifluoroacetate or formate salt, or, in the case of a compound of the present invention, which is sufficiently acidic, an ammonium salt. A salt of this type can either be transformed into its free base or free acid form, respectively, by various methods known to a person skilled in the art or be used as salts in subsequent biological assays. It is to be understood that the specific form of a compound of the present invention as isolated and as described herein is not necessarily the only form in which said compound can be applied to a biological assay in order to quantify the specific biological activity.

Chemical names were generated using the naming function in ChemDraw software (Version 19.0.1.28) by PerkinElmer Informatics, Inc. In some cases, generally accepted names of commercially available reagents were used in place of names generated by the naming software.

### General Methods

¹H NMR (400 MHZ) were obtained in solution of deuteriochloroform (CDCl₃), deuteriomethanol (CD₃OD) or dimethyl sulfoxide—D₆ (DMSO-D₆).

HPLC retention times, purities, and mass spectra (LCMS) were obtained using the following methods:
Method 1: Agilent 1260 Infinity II System equipped with an Agilent Poroshell 120 EC-18, 2.7 μm, 4.6×100 mm column at 30° C., using H₂O with 0.1% FA as the mobile phase A, and CH₃CN with 0.1% FA as the mobile phase B. An ESI detector in positive mode was used. The gradient was 5-95% mobile phase B over 12 min then held at 95% for 1.8 min, then returned to 10% mobile phase B over 0.2 min The flow rate was 1 mL/min.
Method 2: Shimadzu SCL-10A system equipped with Agilent Eclipse XDB-C18, 3.5 μM, 4.6×150 mm column and PE Sciex API 150 EX, using H₂O with 0.1% TFA as the mobile phase A, and MeOH with 0.1% TFA as the mobile phase B. The gradient was 5-95% mobile phase B over 12 min then held at 95% mobile phase B for 3 min, then returned to 5% mobile phase B for 1 min. The flow rate was 1 mL/min.
Method 3: Shimadzu SCL-10A system equipped with Agilent Eclipse XDB-C18, 3.5 μM, 4.6×150 mm column and PE Sciex API 150 EX, using H₂O with 0.1% TFA as the mobile phase A, and MeOH with 0.1% TFA as the mobile phase B. The gradient was 50-95% mobile phase B over 4 min then held at 95% mobile phase B for 4 min, then returned to 50% mobile phase B for 0.1 min. The flow rate was 1 mL/min.
Method 4: Shimadzu LCMS-2020 System equipped with a Kinetex EVO C18 2.1×30 mm, (5 μm particles) column, using H₂O with 0.0375% TFA as the mobile phase A, and CH₃CN with 0.01875% TFA as the mobile phase B. An ESI detector in positive mode was used. The gradient was 5% B at 0.00 min and 5-90% B at 0.00-0.80 min, 90-95% B at 0.80-1.12 min, and then 95-5% B in 0.01 min, hold on 5% B for 0.34 min, the flow rate was 1.5 ml/min.
Method 5: Shimadzu LCMS-2020 System equipped with a Kinetex EVO C18 2.1×30 mm, (5 μm particles) column, using H₂O with 0.025% NH₃·H₂O as the mobile phase A, and CH₃CN as the mobile phase B. An ESI detector in positive mode was used. The gradient was 5% B at 0.00 min and 5-95% B at 0.00-1.2 min, 95-5% B at 1.20-1.21 min, hold on 5% B for 0.34 min, the flow rate was 1.5 ml/min.
Method 6: Shimadzu LCMS-2020 System equipped with a Kinetex EVO C18 2.1×30 mm, (5 μm particles) column, using H₂O with 0.0375% TFA as the mobile phase A, and CH₃CN with 0.01875% TFA as the mobile phase B. An ESI detector in positive mode was used. The gradient was 0% B at 0.00 min and 0-60% B at 0.00-0.80 min, 60-0% B at 0.80-1.20 min, hold on 0% B for 0.34 min, the flow rate was 1.5 ml/min.
Method 7: LCMS-2020 System equipped with a HALO C18 3.0×30 mm, (2.7 μm particles) column, using H₂O with 0.0375% TFA as the mobile phase A, and CH₃CN with 0.01875% TFA as the mobile phase B. An ESI detector in positive mode was used. The gradient was 5% B at 0.00 min and 5-95% B at 0.00-0.50 min, held on 95% B for 0.30 min, 95-5% B at 0.80-0.81 min, and then held on 5% B for 0.24 min, the flow rate was 1.5 ml/min.
Method 8: SHIMADZU LCMS-2020 System equipped with an Xtimate C18 2.1×30 mm, (3 μm particles) column, using H₂O with 0.0375% TFA as the mobile phase A, and CH₃CN with 0.01875% TFA as the mobile phase B. An ESI detector in positive mode was used. The gradient was 10% B at 0.00 min and 10-80% B at 0.00-3.00 min, hold on 80% B for 0.50 min, return to 10% over 0.5 min. The flow rate was 1.2 ml/min. Column temperature was 50° C.
Method 9: SHIMADZU LCMS-2020 System equipped with an Xtimate C18 2.1×30 mm, (3 μm particles) column, using H₂O with 0.0375% TFA as the mobile phase A, and CH₃CN with 0.01875% TFA as the mobile phase B. An ESI detector in positive mode was used. The gradient was 10% B at 0.00 min and 10-80% B at 0.00-0.9 min, hold on 80% B for 0.50 min, return to 10% over 0.5 min. The flow rate was 1.2 ml/min. Column temperature was 50° C.
Method 10: Agilent 1260 Infinity II System equipped with an Agilent Poroshell 120 EC-18, 2.7 μm, 4.6×100 mm column at 30° C., using H₂O with 0.1% FA as the mobile phase A, and CH₃CN with 0.1% FA as the mobile phase B. An ESI detector in negative mode was used. The gradient was 5-95% mobile phase B over 12 min then held at 95% for 1.8 min, then returned to 10% mobile phase B over 0.2 min The flow rate was 1 mL/min.
Method 11: Agilent 1290 Infinity II System equipped with an Agilent Poroshell 120 EC-18, 1.9 μm, 2.1×50 mm column at 35° C., using H₂O with 0.1% FA as the mobile phase A, and CH₃CN with 0.1% FA as the mobile phase B. An ESI detector in positive mode was used. The gradient was 20-95% mobile phase B over 0.8 min then held at 95% for 0.7 min. The flow rate was 0.7 mL/min.
Method 12: Agilent 1260 Infinity II System equipped with an Agilent Poroshell 120 EC-18, 2.7 μm, 4.6×100 mm column at 30° C., using H₂O with 0.1% FA as the mobile phase A, and CH₃CN with 0.1% FA as the mobile phase B. An ESI detector in positive mode was used. The gradient was 10-95% mobile phase B over 4.8 min then dropped to 20% B over 0.2 min, then returned to 10% mobile phase B over 2 min The flow rate was 1 mL/min.

The pyridine, dichloromethane (DCM), tetrahydrofuran (THF), acetonitrile, DMF, and toluene used in the procedures were from Aldrich Sure-Seal bottles, or similar, and kept under nitrogen (N₂). All reactions were stirred magnetically, and temperatures are external reaction temperatures. Chromatographies were typically carried out using a Combiflash Rf flash purification system (Teledyne Isco) equipped with Redisep (Teledyne Isco) silica gel (SiO₂) columns or by using a similar system.

Preparative HPLC purifications were typically performed using one of the following systems (or similar): 1) Waters System equipped with a Waters 2489 uv/vis detector, an Aquity QDA detector, a Waters xBridge Prep C18 5 uM OBD, 30×1560 mm column, and eluting with various gradients of H₂O/CH₃CN (0.1% FA) at a 30 mL/min flow rate, or 2) column: Phenomenex Synergi C18 150×30 mm−4 μm; mobile phase: [H₂O(0.225% FA)-CH₃CN]; B %: 55%-85%, 12 min) and desired fractions were typically concentrated using a Genevac EZ-2.

The following abbreviations are used: ethyl acetate (EA), triethylamine (TEA), dimethylformamide (DMF), diisopropylethylamine (DIEA), dichloromethane (DCM), methanol (MeOH), petroleum ether (pet ether), tetrahydrofuran (THF), acetonitrile (ACN), trifluoracetic acid (TFA), hexafluorophosphate azabenzotriazole tetramethyl 135ranium (HATU), 2-(1H-Benzotriazole-1-yl)-1,1,3,3-tetramethylaminium tetrafluoroborate (TBTU), propanephosphonic acid anhydride (T₃P), dimethylsufoxide (DMSO), dichloroethane (DCE), carbonyl diimidazole (CDI), methyl N-(triethylammoniumsulfonyl)carbamate (Burgess Reagent), N-bromosuccinimide (NBS), N-iodosuccinimide (NIS), liquid chromatography-mass spectrometer (LCMS), nuclear magnetic resonance (NMR), deuterated chloroform (CDCl₃), trifluoroacetic acid (TFA), reverse phase (RP), hour/hours (h), minute (min), 1,8-Diazabicyclo[5.4.0]undec-7-ene (DBU), p-toluenesulfonyl azide (N₃Ts), Hertz (Hz), retention time (t_{R}), trimethylsilyl azide (TMSN₃), petroleum ether (pet ether), acetonitrile (CH₃CN), intermediate (INT), 1,1′-Bis(diphenylphosphino)ferrocene (dppf), retention factor (R_{f}), formic acid (FA), water (H₂O). Other abbreviations commonly known to the art may be included.

### Example 1

### Synthesis of Compound 1-2 and Other Representative Compounds

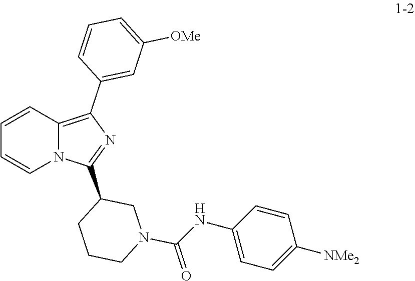 [SMILES: COc1cccc(-c2nc([C@@H]3CCCN(C(=O)Nc4ccc(N(C)C)cc4)C3)n3ccccc23)c1]

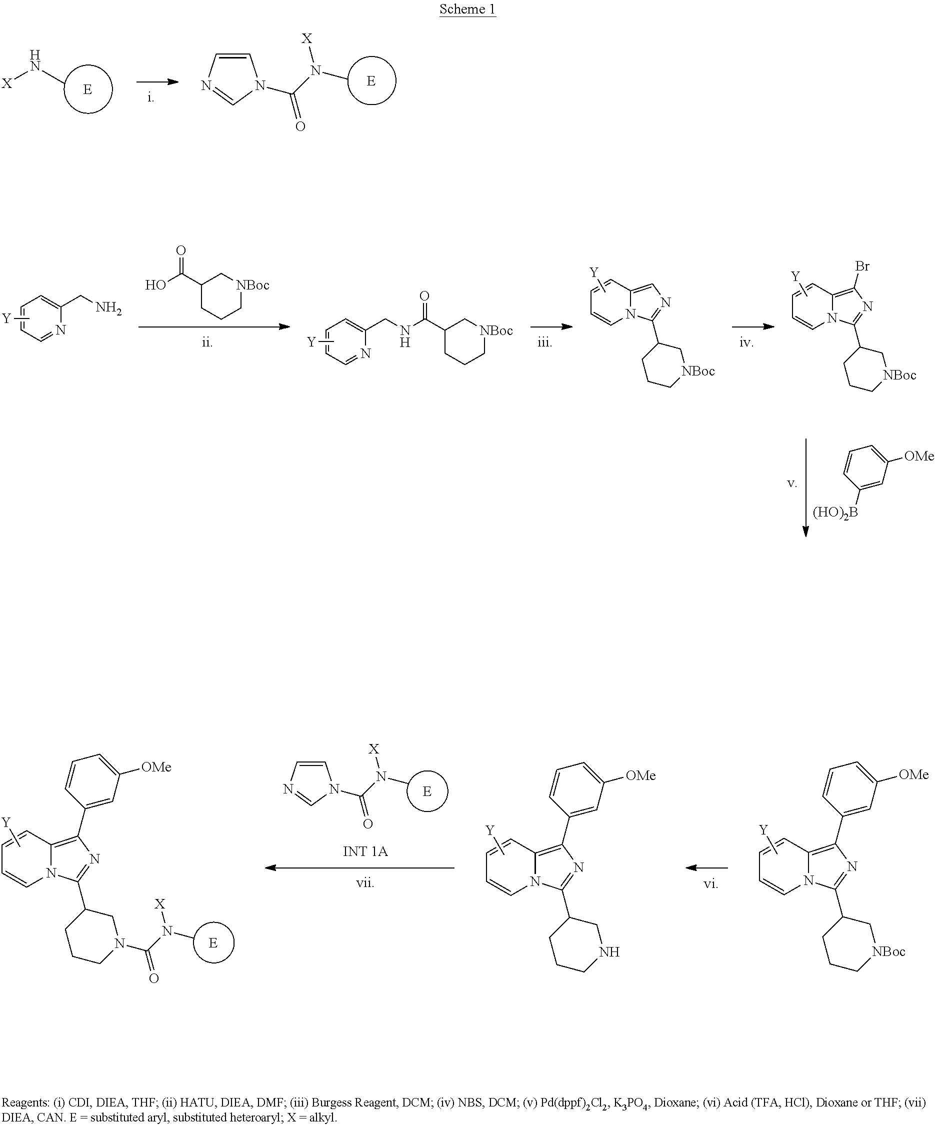

### Step 1-1. N-(4-(dimethylamino)phenyl)-1H-imidazole-1-carboxamide (INT 1A)

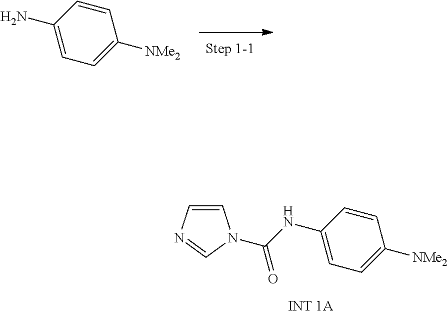 [SMILES: CN(C)c1ccc(N)cc1 ; CN(C)c1ccc(NC(=O)n2ccnc2)cc1]

To a solution of CDI (39.3 mg, 0.24 mmol) in THF (10 mL) were added N1,N1-dimethylbenzene-1,4-diamine (30 mg, 0.22 mmol) and DIEA (56.8 mg, 0.44 mmol). After stirring overnight, the mixture was concentrated to provide 50.7 mg (100%) of crude N-(4-(dimethylamino)phenyl)-1H-imidazole-1-carboxamide (INT 1A) that was used in the next step without further purification.

### Step 1-2. tert-butyl (R)-3-((pyridin-2-ylmethyl)carbamoyl)piperidine-1-carboxylate (INT 1B)

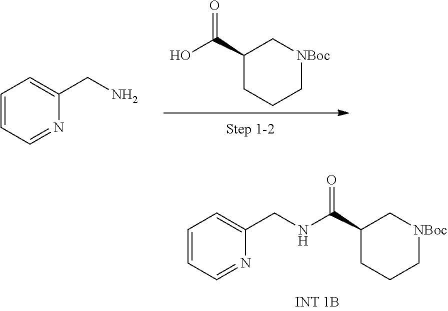 [SMILES: NCc1ccccn1 ; CC(C)(C)OC(=O)N1CCC[C@@H](C(=O)O)C1 ; CC(C)(C)OC(=O)N1CCC[C@@H](C(=O)NCc2ccccn2)C1]

To a solution of (R)-1-(tert-butoxycarbonyl)piperidine-3-carboxylic acid (2.3 g, 10.2 mmol) in DMF (4.9 mL) were added HATU (3.9 g, 10.2 mmol), pyridin-2-ylmethanamine (1.0 g, 9.25 mmol), and DIEA (4.9 mL, 37.9 mmol). After stirring overnight, NaHCO₃ (sat aq) was added, and the mixture was extracted with EA. The organic layer was washed with H₂O and brine, then dried (Na₂SO₄), filtered, and concentrated. The resulting crude material was purified by SiO₂ chromatography (EA/hexane then MeOH/DCM) to provide 1.1 g (37%) of tert-butyl (R)-3-((pyridin-2-ylmethyl)carbamoyl)piperidine-1-carboxylate (INT 1B). LCMS-ESI (m/z) calculated for C₁₇H₂₅N₃O₃: 319.2; found 320.4 [M+H]⁺, t_{R}=5.3 min (Method 3).

### Step 1-3. tert-butyl (R)-3-(imidazo[1,5-a]pyridin-3-yl)piperidine-1-carboxylate (INT 1C)

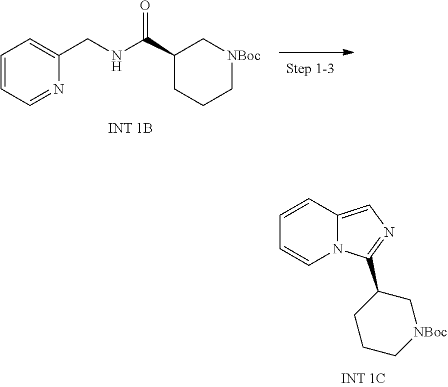 [SMILES: CC(C)(C)OC(=O)N1CCC[C@@H](C(=O)NCc2ccccn2)C1 ; CC(C)(C)OC(=O)N1CCC[C@@H](c2ncc3ccccn23)C1]

To a solution of INT 1B (1.1 g, 3.44 mmol) in DCM (10 mL) was added Burgess Reagent (903 mg, 3.8 mmol). After stirring 18 h, the reaction mixture was concentrated onto SiO₂ gel and was purified by SiO₂ chromatography (EA/hexane, SiO₂ gel preconditioned with 5% NEt₃/hexanes) to provide 480 mg (46%) of tert-butyl (R)-3-(imidazo[1,5-a]pyridin-3-yl)piperidine-1-carboxylate (INT 1C). LCMS-ESI (m/z) calculated for C₁₇H₂₃N₃O₂: 301.4; found 302.4 [M+H]⁺, t_{R}=2.55 min (Method 3).

### Step 1-4. tert-butyl (R)-3-(1-bromoimidazo[1,5-a]pyridin-3-yl)piperidine-1-carboxylate (INT 1D)

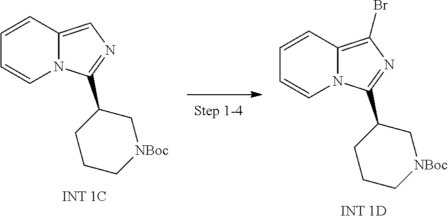 [SMILES: CC(C)(C)OC(=O)N1CCC[C@@H](c2ncc3ccccn23)C1 ; CC(C)(C)OC(=O)N1CCC[C@@H](c2nc(Br)c3ccccn23)C1]

To a solution of INT 1C (480 mg, 1.6 mmol) in DCM (10 mL) was added NBS (312 mg, 1.8 mmol). After stirring 16 h, the reaction mixture was concentrated onto SiO₂ gel and was purified by SiO₂ chromatography (EA/hexane, SiO₂ gel preconditioned with 5% NEt₃/hexanes) to provide 410 mg (68%) of tert-butyl (R)-3-(1-bromoimidazo[1,5-a]pyridin-3-yl)piperidine-1-carboxylate (INT 1D). LCMS-ESI (m/z) calculated for C₁₇H₂₂BrN₃O₂: 379.1; found 380.1 [M+H]⁺, t_{R}=4.88 min (Method 3).

### Step 1-5. tert-butyl (R)-3-(1-(3-methoxyphenyl)imidazo[1,5-a]pyridin-3-yl)piperidine-1-carboxylate (INT 1E)

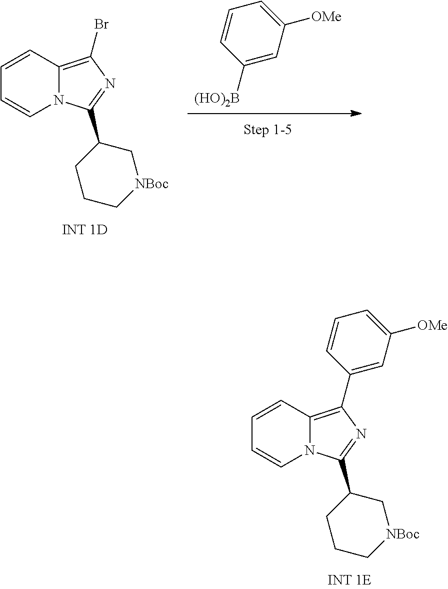 [SMILES: CC(C)(C)OC(=O)N1CCC[C@@H](c2nc(Br)c3ccccn23)C1 ; COc1cccc(B(O)O)c1 ; COc1cccc(-c2nc([C@@H]3CCCN(C(=O)OC(C)(C)C)C3)n3ccccc23)c1]

To a solution of INT 1D (410 mg, 1.1 mmol) in dioxane (6 mL), in a sealable tube, were added K₃PO₄ (686 mg, 3.2 mmol), (3-methoxyphenyl)boronic acid (163.8 mg, 1.1 mmol), and Pd(dppf)₂Cl₂ (78.8 mg, 0.11 mmol). The tube was sealed and heated to 80° C. for 16 hr. The tube was opened, and the reaction mixture was diluted with EA and washed with H₂O, saturated NaHCO₃ (aq) and brine, then dried (Na₂SO₄), filtered and concentrated. The resulting crude residue was purified by SiO₂ chromatography (EA/hexane, SiO₂ gel preconditioned with 5% NEt₃/hexanes) to provide 290 mg (66%) of tert-butyl (R)-3-(1-(3-methoxyphenyl)imidazo[1,5-a]pyridin-3-yl)piperidine-1-carboxylate (INT 1E). LCMS-ESI (m/z) calculated for C₂₄H₂₉N₃O₃: 407.2; found 408.4 [M+H]⁺, t_{R}=4.55 min (Method 3).

### Step 1-6. (R)-1-(3-methoxyphenyl)-3-(piperidin-3-yl)imidazo[1,5-a]pyridine (INT 1F)

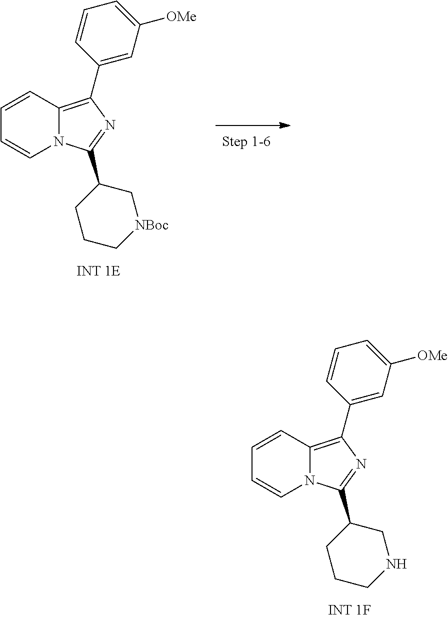 [SMILES: COc1cccc(-c2nc([C@@H]3CCCN(C(=O)OC(C)(C)C)C3)n3ccccc23)c1 ; COc1cccc(-c2nc([C@@H]3CCCNC3)n3ccccc23)c1]

To a solution of INT 1E (170 mg, 0.42 mmol) in DCM (3 mL) was added TFA (3 mL). After 3 h, the reaction mixture was concentrated, diluted with EA and washed with saturated NaHCO₃ (aq) and brine, then dried (Na₂SO₄), filtered and concentrated to provide 120 mg (94%) of (R)-1-(3-methoxyphenyl)-3-(piperidin-3-yl)imidazo[1,5-a]pyridine (INT 1F). LCMS-ESI (m/z) calculated for C₁₉H₂₁N₃O: 307.2; found 308.3 [M+H]⁺, t_{R}=3.46 min (Method 3).

### Step 1-7 (R)—N-(4-(dimethylamino)phenyl)-3-(1-(3-methoxyphenyl)imidazo[1,5-a]pyridin-3-yl)piperidine-1-carboxamide (Compound 1-2)

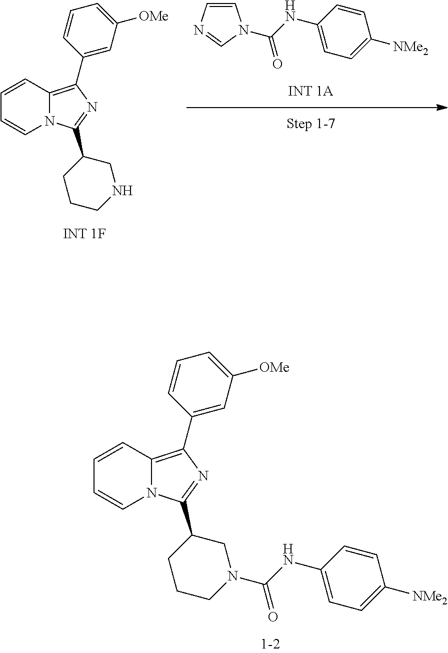 [SMILES: COc1cccc(-c2nc([C@@H]3CCCNC3)n3ccccc23)c1 ; CN(C)c1ccc(NC(=O)n2ccnc2)cc1 ; COc1cccc(-c2nc([C@@H]3CCCN(C(=O)Nc4ccc(N(C)C)cc4)C3)n3ccccc23)c1]

To a solution of INT 1F (55 mg, 0.18 mmol) in ACN (10 mL) were added DIEA (69.2 mg, 0.54 mmol) and INT 1A (82.4 mg, 0.36 mmol). After stirring for 16 h at rt, the reaction mixture was concentrated and purified twice by SiO₂ chromatography (MeOH/DCM), then further purified by Reverse-Phase C18 chromatography (H₂O/MeOH with 0.1% TFA) to provide 24 mg (30%) of (R)—N-(4-(dimethylamino)phenyl)-3-(1-(3-methoxyphenyl)imidazo[1,5-a]pyridin-3-yl)piperidine-1-carboxamide (Compound 1-2). LCMS-ESI (m/z) calculated for C28H₃₁N₅O₂: 469.2; found 470.7 [M+H]⁺, t_{R}=9.24 min (Method 2).

The compounds listed in Table 1 were made using the procedures of Scheme 1.

TABLE 1

| | | Purity | | | | |
|---|---|---|---|---|---|---|
| | Cpd | RT | | Observed | | Purity |
| Structure | No. | (min) | MW | m/z | Ion | Method |
| 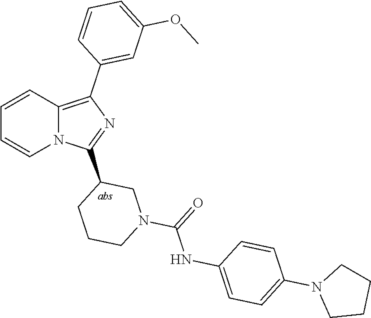 [SMILES: COc1cccc(-c2nc([C@@H]3CCCN(C(=O)Nc4ccc(N5CCCC5)cc4)C3)n3ccccc23)c1] | 1-1 | 9.47 | 495.3 | 496.6 | [M + H]⁺ | 2 |
| 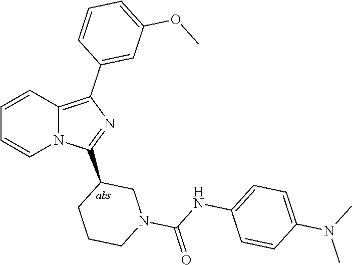 [SMILES: COc1cccc(-c2nc([C@@H]3CCCN(C(=O)Nc4ccc(N(C)C)cc4)C3)n3ccccc23)c1] | 1-2 | 10.51 | 469.2 | 470.7 | [M + H]⁺ | 2 |
| 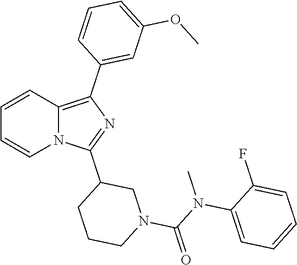 [SMILES: COc1cccc(-c2nc(C3CCCN(C(=O)N(C)c4ccccc4F)C3)n3ccccc23)c1] | 1-3 | 0.907 | 458.2 | 459.2 | [M + H]⁺ | 5 |
| 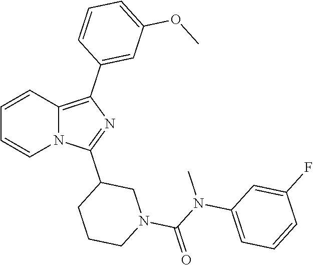 [SMILES: COc1cccc(-c2nc(C3CCCN(C(=O)N(C)c4cccc(F)c4)C3)n3ccccc23)c1] | 1-4 | 0.919 | 458.2 | 459.2 | [M + H]⁺ | 5 |
| 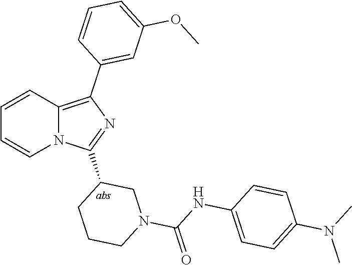 [SMILES: COc1cccc(-c2nc([C@H]3CCCN(C(=O)Nc4ccc(N(C)C)cc4)C3)n3ccccc23)c1] | 1-5 | 10.35 | 469.2 | 470.6 | [M + H]⁺ | 2 |
| 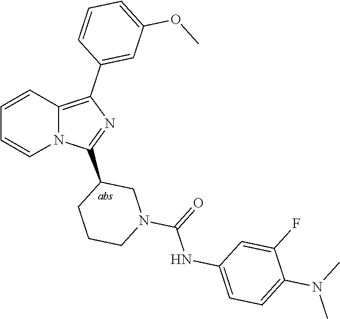 [SMILES: COc1cccc(-c2nc([C@@H]3CCCN(C(=O)Nc4ccc(N(C)C)c(F)c4)C3)n3ccccc23)c1] | 1-6 | 10.57 | 487.2 | 488.5 | [M + H]⁺ | 2 |
| 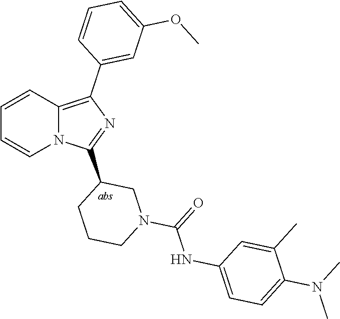 [SMILES: COc1cccc(-c2nc([C@@H]3CCCN(C(=O)Nc4ccc(N(C)C)c(C)c4)C3)n3ccccc23)c1] | 1-7 | 9.5 | 483.3 | 484.6 | [M + H]⁺ | 2 |
| 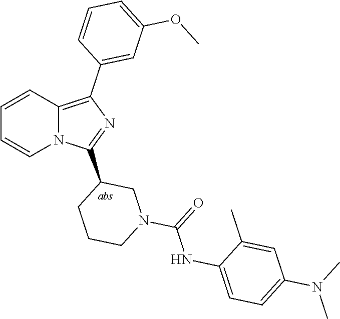 [SMILES: COc1cccc(-c2nc([C@@H]3CCCN(C(=O)Nc4ccc(N(C)C)cc4C)C3)n3ccccc23)c1] | 1-8 | 10.01 | 483.3 | 484.7 | [M + H]⁺ | 2 |
| 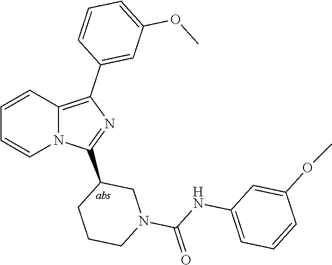 [SMILES: COc1cccc(NC(=O)N2CCC[C@@H](c3nc(-c4cccc(OC)c4)c4ccccn34)C2)c1] | 1-9 | 11.47 | 456.2 | 456.8 | [M + H]⁺ | 2 |
| 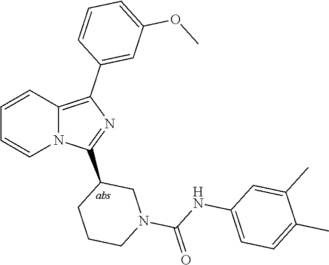 [SMILES: COc1cccc(-c2nc([C@@H]3CCCN(C(=O)Nc4ccc(C)c(C)c4)C3)n3ccccc23)c1] | 1-10 | 12.17 | 454.2 | 455 | [M + H]⁺ | 2 |
| 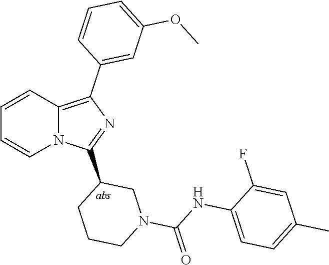 [SMILES: COc1cccc(-c2nc([C@@H]3CCCN(C(=O)Nc4ccc(C)cc4F)C3)n3ccccc23)c1] | 1-11 | 11.92 | 458.2 | 458.7 | [M + H]⁺ | 2 |
| 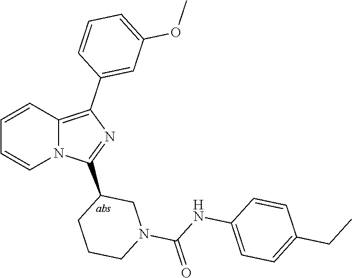 [SMILES: CCc1ccc(NC(=O)N2CCC[C@@H](c3nc(-c4cccc(OC)c4)c4ccccn34)C2)cc1] | 1-12 | 12.12 | 454.2 | 455.2 | [M + H]⁺ | 2 |
| 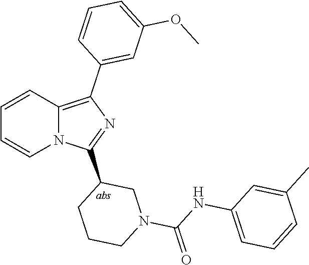 [SMILES: COc1cccc(-c2nc([C@@H]3CCCN(C(=O)Nc4cccc(C)c4)C3)n3ccccc23)c1] | 1-13 | 11.92 | 440.2 | 440.9 | [M + H]⁺ | 2 |
| 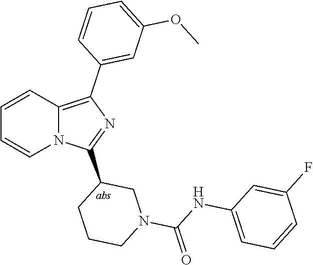 [SMILES: COc1cccc(-c2nc([C@@H]3CCCN(C(=O)Nc4cccc(F)c4)C3)n3ccccc23)c1] | 1-14 | 11.7 | 444.2 | 444.7 | [M + H]⁺ | 2 |
| 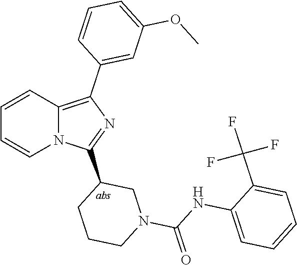 [SMILES: COc1cccc(-c2nc([C@@H]3CCCN(C(=O)Nc4ccccc4C(F)(F)F)C3)n3ccccc23)c1] | 1-15 | 11.54 | 494.2 | 495 | [M + H]⁺ | 2 |
| 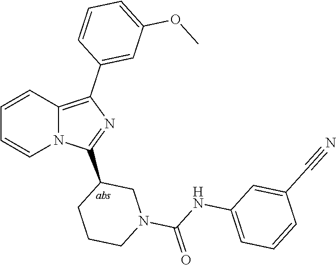 [SMILES: COc1cccc(-c2nc([C@@H]3CCCN(C(=O)Nc4cccc(C#N)c4)C3)n3ccccc23)c1] | 1-16 | 11.43 | 451.2 | 451.7 | [M + H]⁺ | 2 |
| 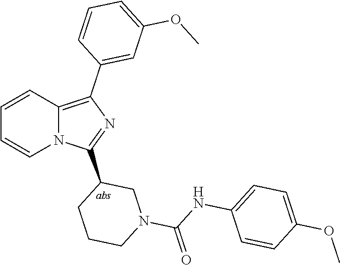 [SMILES: COc1ccc(NC(=O)N2CCC[C@@H](c3nc(-c4cccc(OC)c4)c4ccccn34)C2)cc1] | 1-17 | 11.74 | 456.2 | 456.9 | [M + H]⁺ | 2 |
| 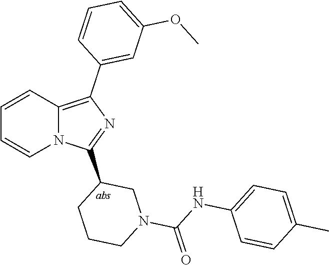 [SMILES: COc1cccc(-c2nc([C@@H]3CCCN(C(=O)Nc4ccc(C)cc4)C3)n3ccccc23)c1] | 1-18 | 11.92 | 440.2 | 440.8 | [M + H]⁺ | 2 |
| 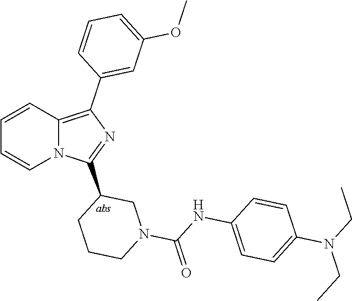 [SMILES: CCN(CC)c1ccc(NC(=O)N2CCC[C@@H](c3nc(-c4cccc(OC)c4)c4ccccn34)C2)cc1] | 1-19 | 10.08 | 497.3 | 497.8 | [M + H]⁺ | 2 |
| 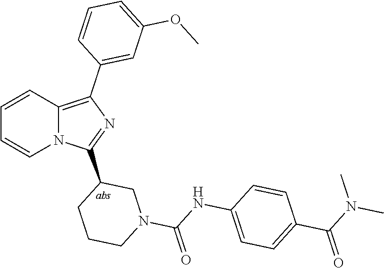 [SMILES: COc1cccc(-c2nc([C@@H]3CCCN(C(=O)Nc4ccc(C(=O)N(C)C)cc4)C3)n3ccccc23)c1] | 1-20 | 10.65 | 497.2 | 497.7 | [M + H]⁺ | 2 |
| 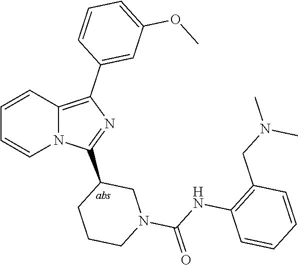 [SMILES: COc1cccc(-c2nc([C@@H]3CCCN(C(=O)Nc4ccccc4CN(C)C)C3)n3ccccc23)c1] | 1-21 | 10.4 | 483.3 | 484.1 | [M + H]⁺ | 2 |
| 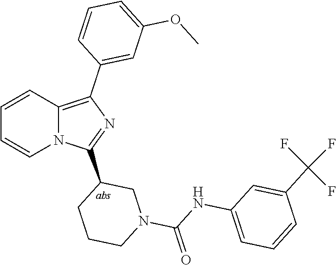 [SMILES: COc1cccc(-c2nc([C@@H]3CCCN(C(=O)Nc4cccc(C(F)(F)F)c4)C3)n3ccccc23)c1] | 1-22 | 12.41 | 494.2 | 494.7 | [M + H]⁺ | 2 |
| 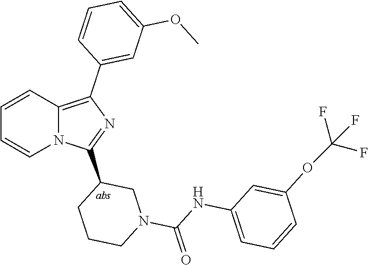 [SMILES: COc1cccc(-c2nc([C@@H]3CCCN(C(=O)Nc4cccc(OC(F)(F)F)c4)C3)n3ccccc23)c1] | 1-23 | 12.55 | 510.2 | 510.6 | [M + H]⁺ | 2 |
| 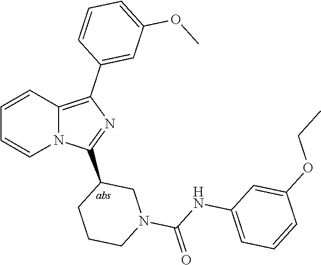 [SMILES: CCOc1cccc(NC(=O)N2CCC[C@@H](c3nc(-c4cccc(OC)c4)c4ccccn34)C2)c1] | 1-24 | 11.96 | 470.2 | 470.8 | [M + H]⁺ | 2 |
| 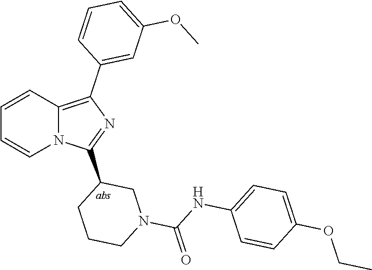 [SMILES: CCOc1ccc(NC(=O)N2CCC[C@@H](c3nc(-c4cccc(OC)c4)c4ccccn34)C2)cc1] | 1-25 | 11.8 | 470.2 | 470.8 | [M + H]⁺ | 2 |
| 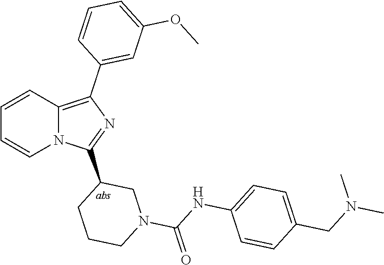 [SMILES: COc1cccc(-c2nc([C@@H]3CCCN(C(=O)Nc4ccc(CN(C)C)cc4)C3)n3ccccc23)c1] | 1-26 | 9.91 | 483.3 | 483.9 | [M + H]⁺ | 2 |
| 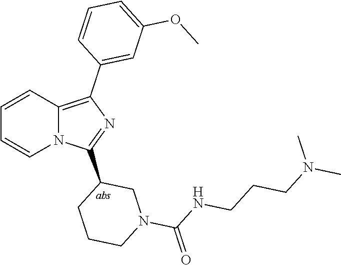 [SMILES: COc1cccc(-c2nc([C@@H]3CCCN(C(=O)NCCCN(C)C)C3)n3ccccc23)c1] | 1-27 | 9.95 | 435.3 | 435.9 | [M + H]⁺ | 2 |
| 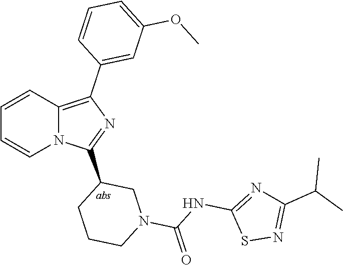 [SMILES: COc1cccc(-c2nc([C@@H]3CCCN(C(=O)Nc4nc(C(C)C)ns4)C3)n3ccccc23)c1] | 1-28 | 12.44 | 476.2 | 476.7 | [M + H]⁺ | 2 |
| 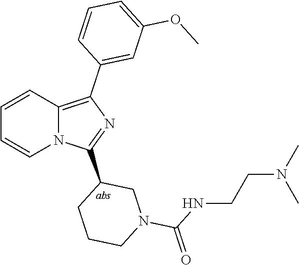 [SMILES: COc1cccc(-c2nc([C@@H]3CCCN(C(=O)NCCN(C)C)C3)n3ccccc23)c1] | 1-29 | 9.3 | 421.2 | 421.9 | [M + H]⁺ | 2 |
| 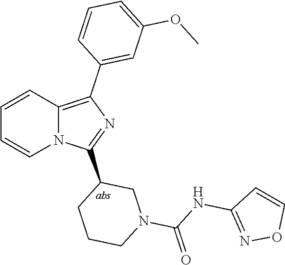 [SMILES: COc1cccc(-c2nc([C@@H]3CCCN(C(=O)Nc4ccon4)C3)n3ccccc23)c1] | 1-30 | 10.68 | 417.2 | 417.5 | [M + H]⁺ | 2 |
| 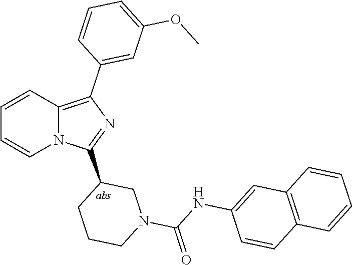 [SMILES: COc1cccc(-c2nc([C@@H]3CCCN(C(=O)Nc4ccc5ccccc5c4)C3)n3ccccc23)c1] | 1-31 | 12.56 | 476.2 | 476.7 | [M + H]⁺ | 2 |
| 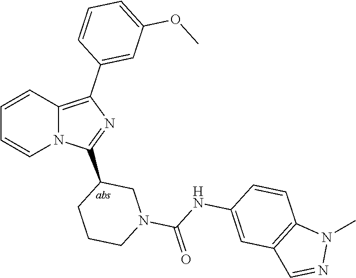 [SMILES: COc1cccc(-c2nc([C@@H]3CCCN(C(=O)Nc4ccc5c(cnn5C)c4)C3)n3ccccc23)c1] | 1-32 | 11.4 | 480.2 | 480.7 | [M + H]⁺ | 2 |
| 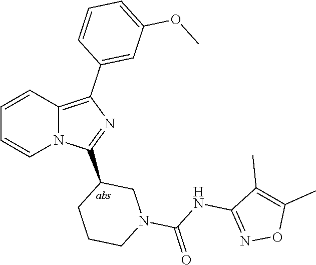 [SMILES: COc1cccc(-c2nc([C@@H]3CCCN(C(=O)Nc4noc(C)c4C)C3)n3ccccc23)c1] | 1-33 | 10.81 | 445.2 | 445.8 | [M + H]⁺ | 2 |

### Example 2

### Synthesis of Compound 2-1 and Other Representative Compounds

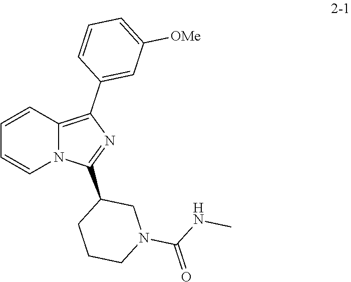 [SMILES: CNC(=O)N1CCC[C@@H](c2nc(-c3cccc(OC)c3)c3ccccn23)C1]

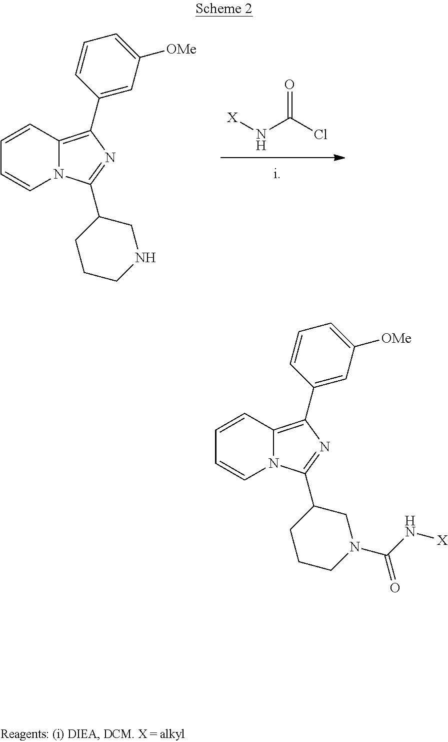

### Step 2-1. Synthesis of (R)-3-(1-(3-methoxyphenyl)imidazo[1,5-a]pyridin-3-yl)-N-methylpiperidine-1-carboxamide (Compound 2-1)

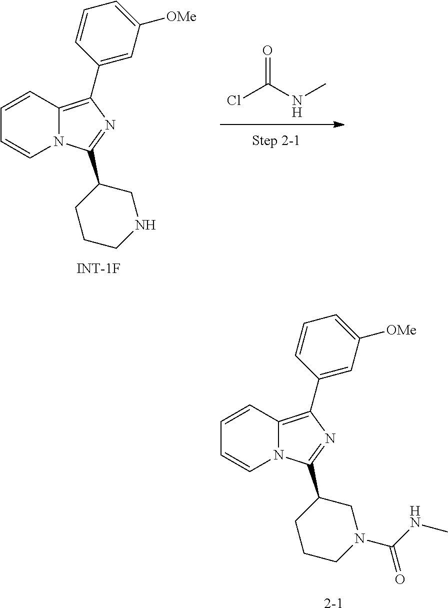

To a stirring solution of INT-1F (60 mg, 0.2 mmol) in DCM (10 mL) were added DIEA (100 mg, 0.78 mmol) and methylcarbamic chloride (20 mg, 0.21 mmol). After 4 hr, additional methylcarbamic chloride (10 mg, 0.1 mmol) was added. After an additional 12 h, the reaction mixture was concentrated, and the resulting crude material was purified by SiO₂ chromatography (EA/hexane (with 5% TEA) to provide material that was further purified by reverse-phase chromatography (MeOH/H₂O with 0.1% TFA) to provide 35 mg (38%) of (R)-3-(1-(3-methoxyphenyl)imidazo[1,5-a]pyridin-3-yl)-N-methylpiperidine-1-carboxamide (Compound 2-1). LCMS-ESI (m/z) calculated for C₂₁H₂₄N₄O₂: 364.2; found 365.3 [M+H]⁺, t_{R}=9.61 min (Method 2).

The compounds listed in Table 2 were made using the procedures of Scheme 2.

TABLE 2

| | | Purity | | | | |
|---|---|---|---|---|---|---|
| | Cpd | RT | | Observed | | Purity |
| Structure | No. | (min) | MW | m/z | Ion | Method |
| 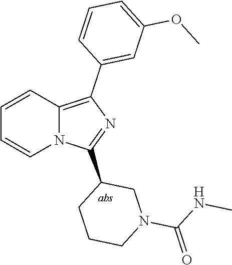 [SMILES: CNC(=O)N1CCC[C@@H](c2nc(-c3cccc(OC)c3)c3ccccn23)C1] | 2-1 | 10.42 | 364.2 | 365.3 | [M + H]⁺ | 2 |
| 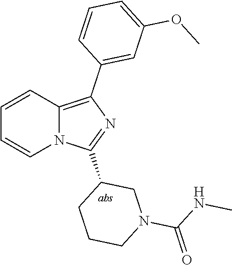 [SMILES: CNC(=O)N1CCC[C@H](c2nc(-c3cccc(OC)c3)c3ccccn23)C1] | 2-2 | 10.47 | 364.2 | 365.3 | [M + H]⁺ | 2 |

### Example 3

### Synthesis of Compound 3-1 and Other Representative Compounds

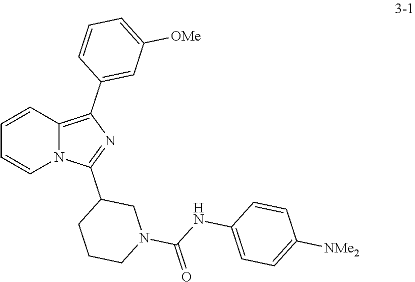 [SMILES: COc1cccc(-c2nc(C3CCCN(C(=O)Nc4ccc(N(C)C)cc4)C3)n3ccccc23)c1]

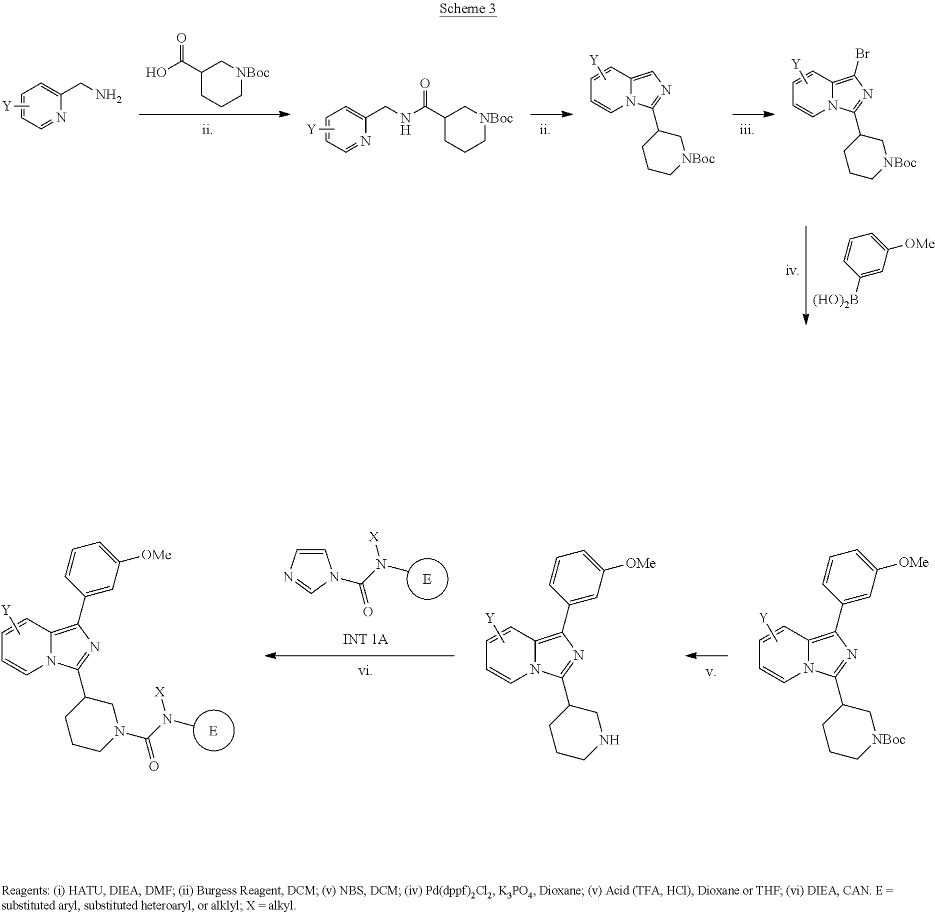

### Step 3-1. tert-butyl 3-((pyridin-2-ylmethyl)carbamoyl)piperidine-1-carboxylate (INT 3B)

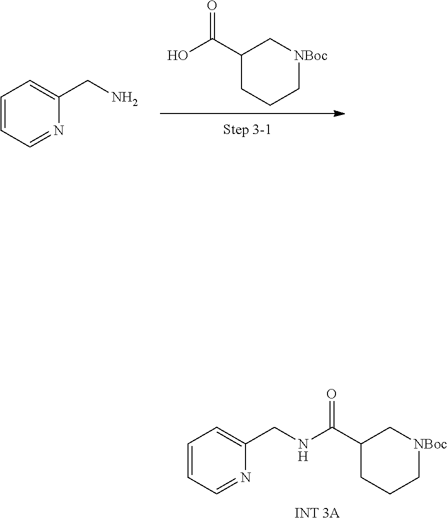 [SMILES: NCc1ccccn1 ; CC(C)(C)OC(=O)N1CCCC(C(=O)O)C1 ; CC(C)(C)OC(=O)N1CCCC(C(=O)NCc2ccccn2)C1 ; "INT 3A"]

To a solution of 1-(tert-butoxycarbonyl)piperidine-3-carboxylic acid (53 g, 231 mmol) and pyridin-2-ylmethanamine (25.0 g, 231 mmol) in DMF (1000 mL) were added DIEA (80.5 mL, 462 mmol) and HATU (175.8 g, 462.4 mmol). After stirring at 20° C. for 12 h, the mixture was concentrated under reduced pressure to remove the DMF. The resulting residue was diluted with H₂O and extracted with EA (3×). The combined organic layers were washed with brine, then dried (Na₂SO₄), filtered, and concentrated. The resulting crude material was purified by SiO₂ chromatography (EA/petroleum ether) to provide 52 g (63.4%) of tert-butyl 3-((pyridin-2-ylmethyl)carbamoyl)piperidine-1-carboxylate (INT 3A) as a brown oil. LCMS-ESI (m/z) calculated for C₁₇H₂₅N₃O₃: 319.2; found 320.2[M+H]⁺, t_{R}=0.769 min (Method 4). ¹H NMR (400 MHz, CDCl₃): δ=1.45 (s, 9H) 1.66-2.01 (m, 3H) 2.39 (br d, J=8.07 Hz, 1H) 3.09-3.31 (m, 2H) 3.73 (dtd, J=13.24, 6.59, 6.59, 4.28 Hz, 1H) 3.99-4.11 (m, 1H) 4.47-4.70 (m, 2H) 7.00 (br d, J=2.93 Hz, 1H) 7.24 (br d, J=7.34 Hz, 1H) 7.71 (td, J=7.64, 1.59 Hz, 1H) 8.03 (s, 1H) 8.54 (d, J=4.40 Hz, 1H).

### Step 3-2. tert-butyl 3-(imidazo[1,5-a]pyridin-3-yl)piperidine-1-carboxylate (INT 3B)

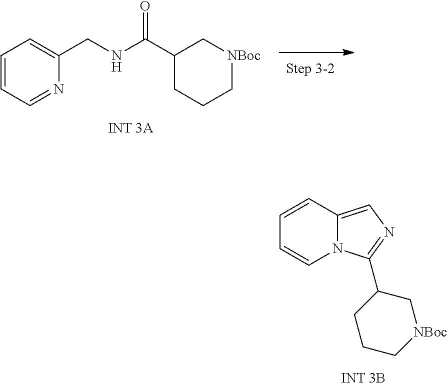 [SMILES: CC(C)(C)OC(=O)N1CCCC(C(=O)NCc2ccccn2)C1 ; CC(C)(C)OC(=O)N1CCCC(c2ncc3ccccn23)C1]

To a solution of INT 3A (20 g, 56 mmol) in DCM (100 mL) was added Burgess Reagent (14.8 g, 62 mmol). After stirring 8 h at 25° C., the reaction mixture was diluted with H₂O and extracted with DCM (3×). The combined organic layers were washed with brine (2×), dried (Na₂SO₄), filtered and concentrated to provide a residue that was purified by SiO₂ chromatography (EA/petroleum ether) to provide 13 g (61%) of tert-butyl-3-(imidazo[1,5-a]pyridin-3-yl)piperidine-1-carboxylate (INT 3B) as a yellow oil. TLC (1:1 petroleum ether:EA) R_{f} 0.48 (UV254).

### Step 3-3. tert-butyl-3-(1-bromoimidazo[1,5-a]pyridin-3-yl)piperidine-1-carboxylate (INT 3C)

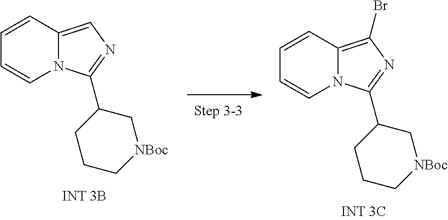 [SMILES: CC(C)(C)OC(=O)N1CCCC(c2ncc3ccccn23)C1 ; CC(C)(C)OC(=O)N1CCCC(c2nc(Br)c3ccccn23)C1]

To a solution of INT 3B (7.4 g, 41 mmol) in DCM (150 mL) was added NBS (7.4 g, 41 mmol). After stirring 12 h at 20° C., the reaction mixture was diluted with H₂O and extracted with DCM (3×). The combined organic layers were washed with brine (2×), dried (Na₂SO₄), filtered and concentrated to provide a residue that was purified by SiO₂ chromatography (EA/petroleum ether) to provide 11 g (59%) of tert-butyl-3-(1-bromoimidazo[1,5-a]pyridin-3-yl)piperidine-1-carboxylate (INT 3C) as a gray solid. LCMS-ESI (m/z) calculated for C₁₇H₂₂BrN₃O₂: 379.1; found 380.2 [M+H]⁺, t_{R}=0.823 min (Method 4).

### Step 3-4. tert-butyl-3-(1-(3-methoxyphenyl)imidazo[1,5-a]pyridin-3-yl)piperidine-1-carboxylate (INT 3D)

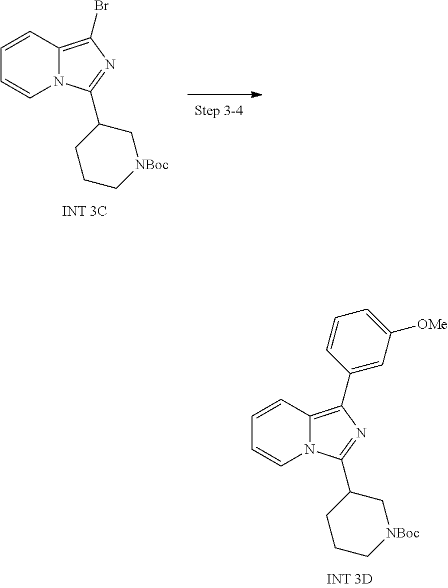 [SMILES: CC(C)(C)OC(=O)N1CCCC(c2nc(Br)c3ccccn23)C1 ; COc1cccc(-c2nc(C3CCCN(C(=O)OC(C)(C)C)C3)n3ccccc23)c1]

To a solution of INT 3D (2 g, 3.7 mmol) in dioxane (4 mL) and H₂O (0.5 mL) were added Cs₂CO₃ (3.0 g, 9.2 mmol), (3-methoxyphenyl)boronic acid (671 mg, 4.4 mmol), and Pd(dppf)₂Cl₂ (539 mg, 0.74 mmol). The mixture was degassed and heated to 90° C. for 12 hr under an atmosphere of N₂. The reaction mixture was diluted with H₂O and extracted with EA (3×). The combined organic layers were washed with brine (2×), then dried (Na₂SO₄), filtered and concentrated. The resulting crude residue was purified by SiO₂ chromatography (EA/petroleum ether) to provide 1.2 g (72%) of tert-butyl-3-(1-(3-methoxyphenyl)imidazo[1,5-a]pyridin-3-yl)piperidine-1-carboxylate (INT 3D) as a yellow oil. LCMS-ESI (m/z) calculated for C₂₄H₂₉N₃O₃: 407.2; found 408.3 [M+H]⁺, t_{R}=0.727 min (Method 4).

### Step 3-5. 1-(3-methoxyphenyl)-3-(piperidin-3-yl)imidazo[1,5-a]pyridine (INT 3E)

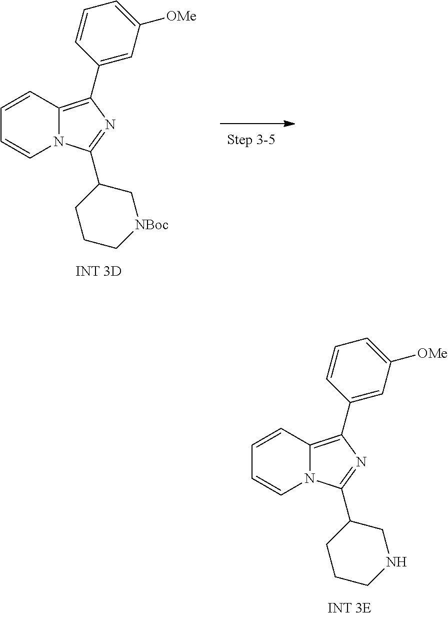 [SMILES: COc1cccc(-c2nc(C3CCCN(C(=O)OC(C)(C)C)C3)n3ccccc23)c1 ; COc1cccc(-c2nc(C3CCCNC3)n3ccccc23)c1]

A solution of INT 3D (1.2 g, 2.65 mmol) in EA (100 mL) with added HCl (10 mL) was stirred at 20° C. for 2 h. The reaction mixture was concentrated to provide 900 mg (89%) of the HCl salt of 1-(3-methoxyphenyl)-3-(piperidin-3-yl)imidazo[1,5-a]pyridine (INT 3E) as a white solid that was used without further purification. LCMS-ESI (m/z) calculated for C₁₉H₂₁N₃O: 307.2; found 308.3 [M+H]⁺, t_{R}=1.26 min (Method 6).

### Step 3-6 N-(4-(dimethylamino)phenyl)-3-(1-(3-methoxyphenyl)imidazo[1,5-a]pyridin-3-yl)piperidine-1-carboxamide (Compound 3-25)

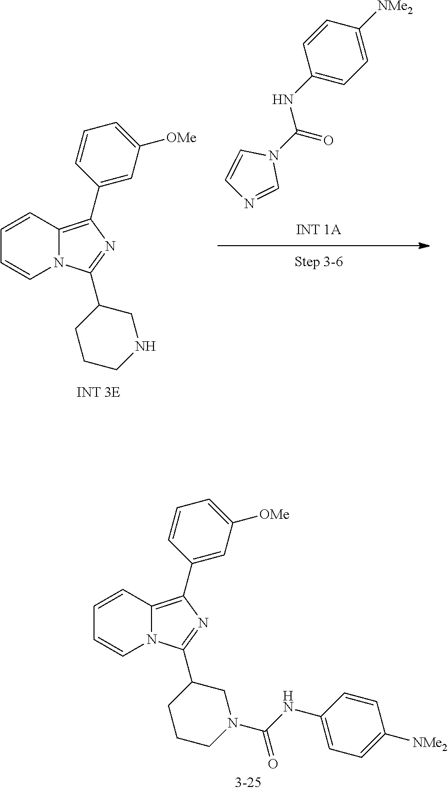 [SMILES: COc1cccc(-c2nc(C3CCCNC3)n3ccccc23)c1 ; CN(C)c1ccc(NC(=O)n2ccnc2)cc1 ; COc1cccc(-c2nc(C3CCCN(C(=O)Nc4ccc(N(C)C)cc4)C3)n3ccccc23)c1]

To a solution of INT 3E (100 mg, 0.33 mmol) in ACN (3 mL) were added DIEA (170 μL, 0.17 mmol) and INT 1A (82 mg, 0.36 mmol). After stirring for 12 h at 80° C., the reaction mixture was concentrated and diluted with H₂O. The mixture was extracted with EA (3×). The combined organic layers were washed with brine, dried (Na₂SO₄), and concentrated to provide a residue that was purified by prep HPLC chromatography (H₂O (10 mM NH₄HCO₃)/ACN) to provide 54 mg (35%) of N-(4-(dimethylamino)phenyl)-3-(1-(3-methoxyphenyl)imidazo[1,5-a]pyridin-3-yl)piperidine-1-carboxamide (Compound 3-25) as a pale yellow solid. LCMS-ESI (m/z) calculated for C28H₃₁N₅O₂: 469.2; found 470.3 [M+H]⁺, t_{R}=1.31 min (Method 5). ¹HNMR (400 MHZ, CDCl₃) δ 1.57-1.85 (m, 2H), 2.15-2.26 (m, 2H), 2.78-2.89 (m, 6H), 3.15-3.38 (m, 3H), 3.78 (s, 4H), 4.25 (br d, J=10.88 Hz, 1H), 6.51 (t, J=6.30 Hz, 1H), 6.60-6.71 (m, 4H), 6.73-6.79 (m, 1H), 7.13 (d, J=8.93 Hz, 2H), 7.23-7.30 (m, 1H), 7.31-7.39 (m, 2H), 7.70 (d, J=9.29 Hz, 1H), 7.86 (d, J=7.21 Hz, 1H).

The compounds listed in Table 3 were made using the procedures of Scheme 3.

TABLE 3

| | | Purity | | | | |
|---|---|---|---|---|---|---|
| | Cpd | RT | | Observed | | Purity |
| Structure | No. | (min) | MW | m/z | Ion | Method |
| 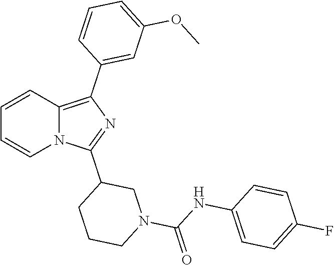 [SMILES: COc1cccc(-c2nc(C3CCCN(C(=O)Nc4ccc(F)cc4)C3)n3ccccc23)c1] | 3-1 | 1.816 | 444.2 | 445.2 | [M + H]⁺ | 8 |
| 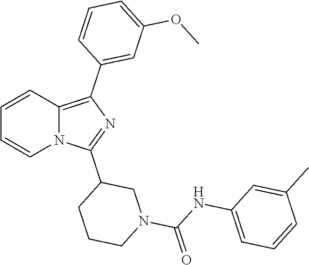 [SMILES: COc1cccc(-c2nc(C3CCCN(C(=O)Nc4cccc(C)c4)C3)n3ccccc23)c1] | 3-2 | 1.895 | 440.2 | 441.2 | [M + H]⁺ | 8 |
| 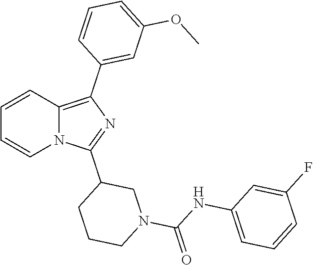 [SMILES: COc1cccc(-c2nc(C3CCCN(C(=O)Nc4cccc(F)c4)C3)n3ccccc23)c1] | 3-3 | 1.881 | 444.2 | 445.2 | [M + H]⁺ | 8 |
| 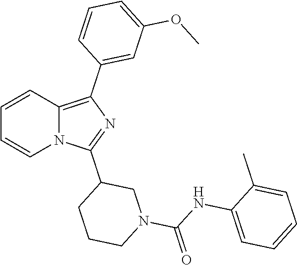 [SMILES: COc1cccc(-c2nc(C3CCCN(C(=O)Nc4ccccc4C)C3)n3ccccc23)c1] | 3-4 | 1.853 | 440.2 | 441.3 | [M + H]⁺ | 8 |
| 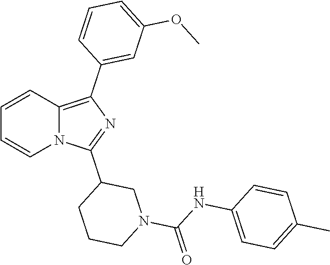 [SMILES: COc1cccc(-c2nc(C3CCCN(C(=O)Nc4ccc(C)cc4)C3)n3ccccc23)c1] | 3-5 | 1.899 | 440.2 | 441.2 | [M + H]⁺ | 8 |
| 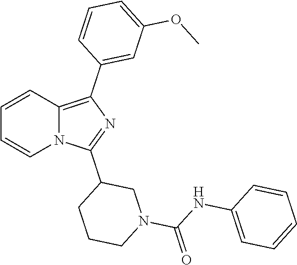 [SMILES: COc1cccc(-c2nc(C3CCCN(C(=O)Nc4ccccc4)C3)n3ccccc23)c1] | 3-6 | 0.838 | 426.2 | 427.2 | [M + H]⁺ | 8 |
| 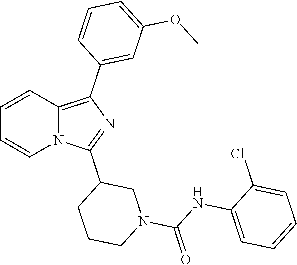 [SMILES: COc1cccc(-c2nc(C3CCCN(C(=O)Nc4ccccc4Cl)C3)n3ccccc23)c1] | 3-7 | 0.899 | 460.2 | 461.2 | [M + H]⁺ | 4 |
| 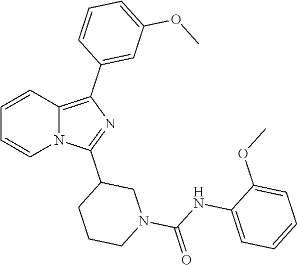 [SMILES: COc1cccc(-c2nc(C3CCCN(C(=O)Nc4ccccc4OC)C3)n3ccccc23)c1] | 3-8 | 0.888 | 456.2 | 457.2 | [M + H]⁺ | 4 |
| 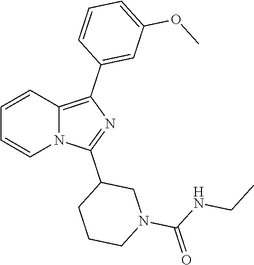 [SMILES: CCNC(=O)N1CCCC(c2nc(-c3cccc(OC)c3)c3ccccn23)C1] | 3-9 | 0.83 | 378.2 | 379.2 | [M + H]⁺ | 4 |
| 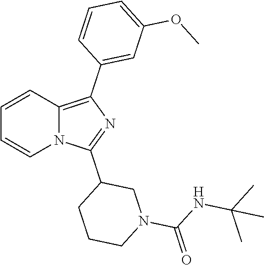 [SMILES: COc1cccc(-c2nc(C3CCCN(C(=O)NC(C)(C)C)C3)n3ccccc23)c1] | 3-10 | 0.898 | 406.2 | 407.2 | [M + H]⁺ | 4 |
| 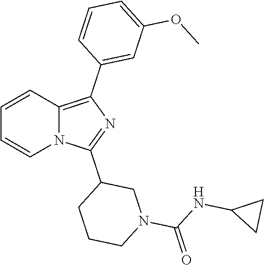 [SMILES: COc1cccc(-c2nc(C3CCCN(C(=O)NC4CC4)C3)n3ccccc23)c1] | 3-11 | 0.814 | 390.2 | 391.2 | [M + H]⁺ | 4 |
| 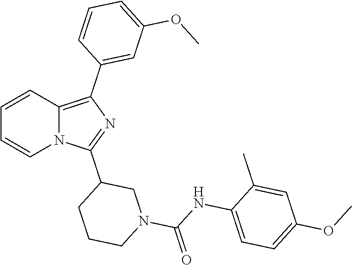 [SMILES: COc1cccc(-c2nc(C3CCCN(C(=O)Nc4ccc(OC)cc4C)C3)n3ccccc23)c1] | 3-12 | 0.874 | 470.2 | 471.2 | [M + H]⁺ | 4 |
| 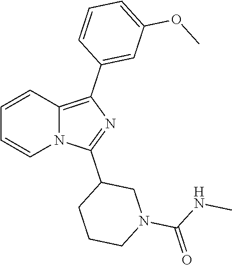 [SMILES: CNC(=O)N1CCCC(c2nc(-c3cccc(OC)c3)c3ccccn23)C1] | 3-13 | 0.781 | 364.2 | 365.2 | [M + H]⁺ | 4 |
| 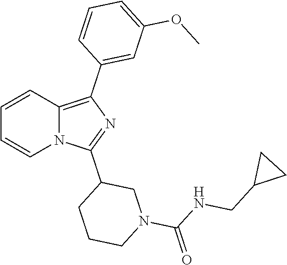 [SMILES: COc1cccc(-c2nc(C3CCCN(C(=O)NCC4CC4)C3)n3ccccc23)c1] | 3-14 | 0.845 | 404.2 | 405.2 | [M + H]⁺ | 4 |
| 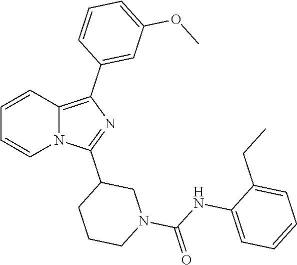 [SMILES: CCc1ccccc1NC(=O)N1CCCC(c2nc(-c3cccc(OC)c3)c3ccccn23)C1] | 3-15 | 0.902 | 454.2 | 455.2 | [M + H]⁺ | 4 |
| 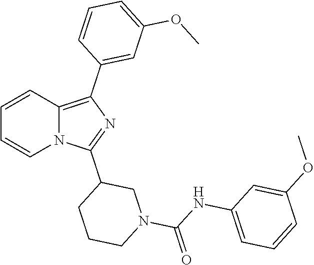 [SMILES: COc1cccc(NC(=O)N2CCCC(c3nc(-c4cccc(OC)c4)c4ccccn34)C2)c1] | 3-16 | 0.879 | 456.2 | 457.2 | [M + H]⁺ | 4 |
| 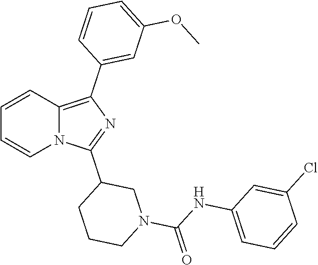 [SMILES: COc1cccc(-c2nc(C3CCCN(C(=O)Nc4cccc(Cl)c4)C3)n3ccccc23)c1] | 3-17 | 0.923 | 460.2 | 461.2 | [M + H]⁺ | 4 |
| 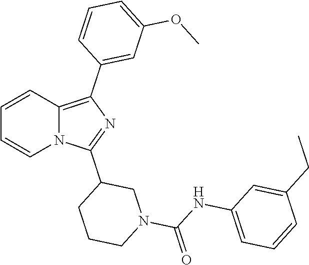 [SMILES: CCc1cccc(NC(=O)N2CCCC(c3nc(-c4cccc(OC)c4)c4ccccn34)C2)c1] | 3-18 | 0.921 | 454.2 | 455.2 | [M + H]⁺ | 4 |
| 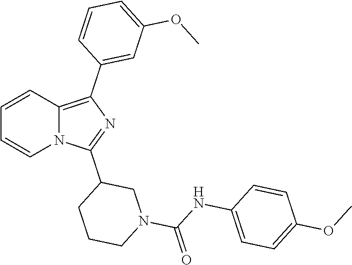 [SMILES: COc1ccc(NC(=O)N2CCCC(c3nc(-c4cccc(OC)c4)c4ccccn34)C2)cc1] | 3-19 | 0.868 | 456.2 | 457.2 | [M + H]⁺ | 4 |
| 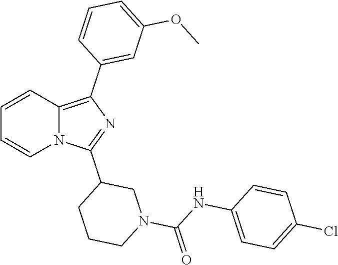 [SMILES: COc1cccc(-c2nc(C3CCCN(C(=O)Nc4ccc(Cl)cc4)C3)n3ccccc23)c1] | 3-20 | 0.915 | 460.2 | 461.2 | [M + H]⁺ | 4 |
| 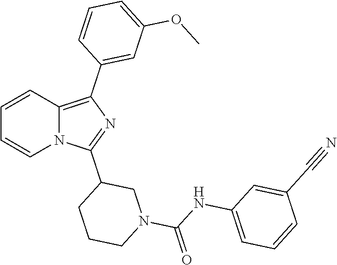 [SMILES: COc1cccc(-c2nc(C3CCCN(C(=O)Nc4cccc(C#N)c4)C3)n3ccccc23)c1] | 3-21 | 0.892 | 451.2 | 452.2 | [M + H]⁺ | 4 |
| 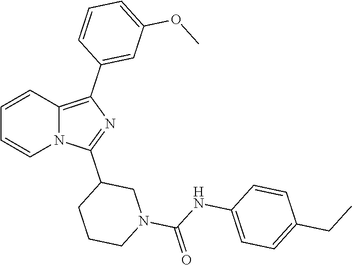 [SMILES: CCc1ccc(NC(=O)N2CCCC(c3nc(-c4cccc(OC)c4)c4ccccn34)C2)cc1] | 3-22 | 0.935 | 454.2 | 455.3 | [M + H]⁺ | 4 |
| 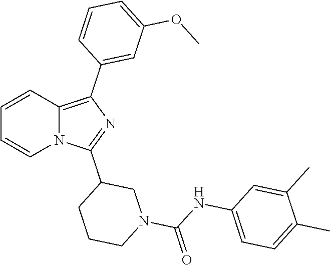 [SMILES: COc1cccc(-c2nc(C3CCCN(C(=O)Nc4ccc(C)c(C)c4)C3)n3ccccc23)c1] | 3-23 | 0.927 | 454.2 | 455.3 | [M + H]⁺ | 4 |
| 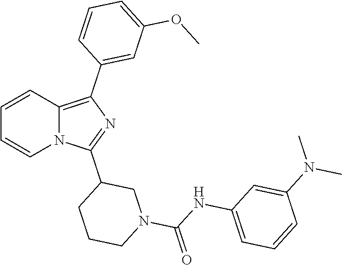 [SMILES: COc1cccc(-c2nc(C3CCCN(C(=O)Nc4cccc(N(C)C)c4)C3)n3ccccc23)c1] | 3-24 | 1.331 | 469.2 | 470.2 | [M + H]⁺ | 5 |
| 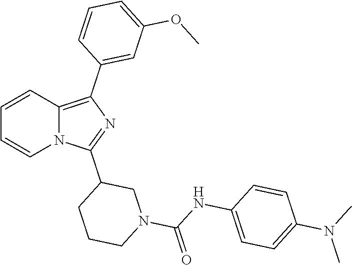 [SMILES: COc1cccc(-c2nc(C3CCCN(C(=O)Nc4ccc(N(C)C)cc4)C3)n3ccccc23)c1] | 3-25 | 1.308 | 469.2 | 470.3 | [M + H]⁺ | 5 |
| 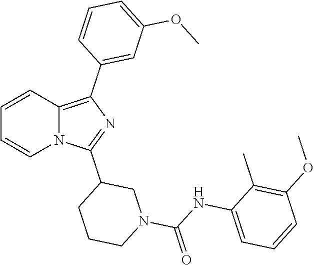 [SMILES: COc1cccc(-c2nc(C3CCCN(C(=O)Nc4cccc(OC)c4C)C3)n3ccccc23)c1] | 3-26 | 1.319 | 470.2 | 471.2 | [M + H]⁺ | 5 |
| 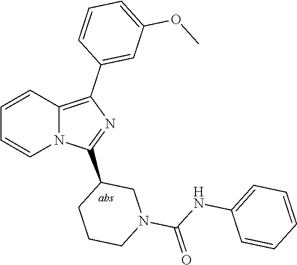 [SMILES: COc1cccc(-c2nc([C@@H]3CCCN(C(=O)Nc4ccccc4)C3)n3ccccc23)c1] | 3-27 | 11.66 | 426.2 | 426.6 | [M + H]⁺ | 2 |
| 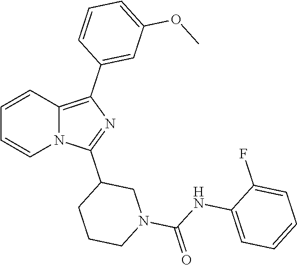 [SMILES: COc1cccc(-c2nc(C3CCCN(C(=O)Nc4ccccc4F)C3)n3ccccc23)c1] | 3-28 | 0.855 | 444.2 | 445.2 | [M + H]⁺ | 8 |
| 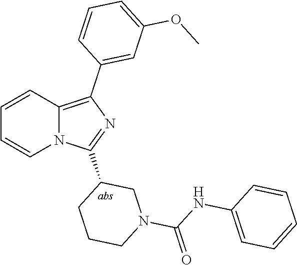 [SMILES: COc1cccc(-c2nc([C@H]3CCCN(C(=O)Nc4ccccc4)C3)n3ccccc23)c1] | 3-29 | 11.12 | 426.2 | 426.9 | [M + H]⁺ | 2 |

### Example 4

### Synthesis of Compound 4-2 and Other Representative Compounds

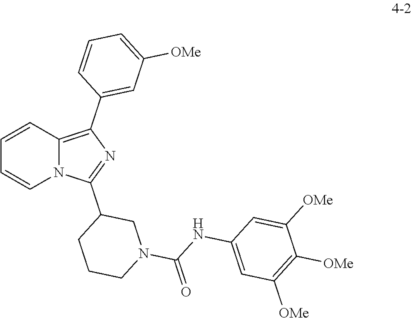 [SMILES: COc1cccc(-c2nc(C3CCCN(C(=O)Nc4cc(OC)c(OC)c(OC)c4)C3)n3ccccc23)c1]

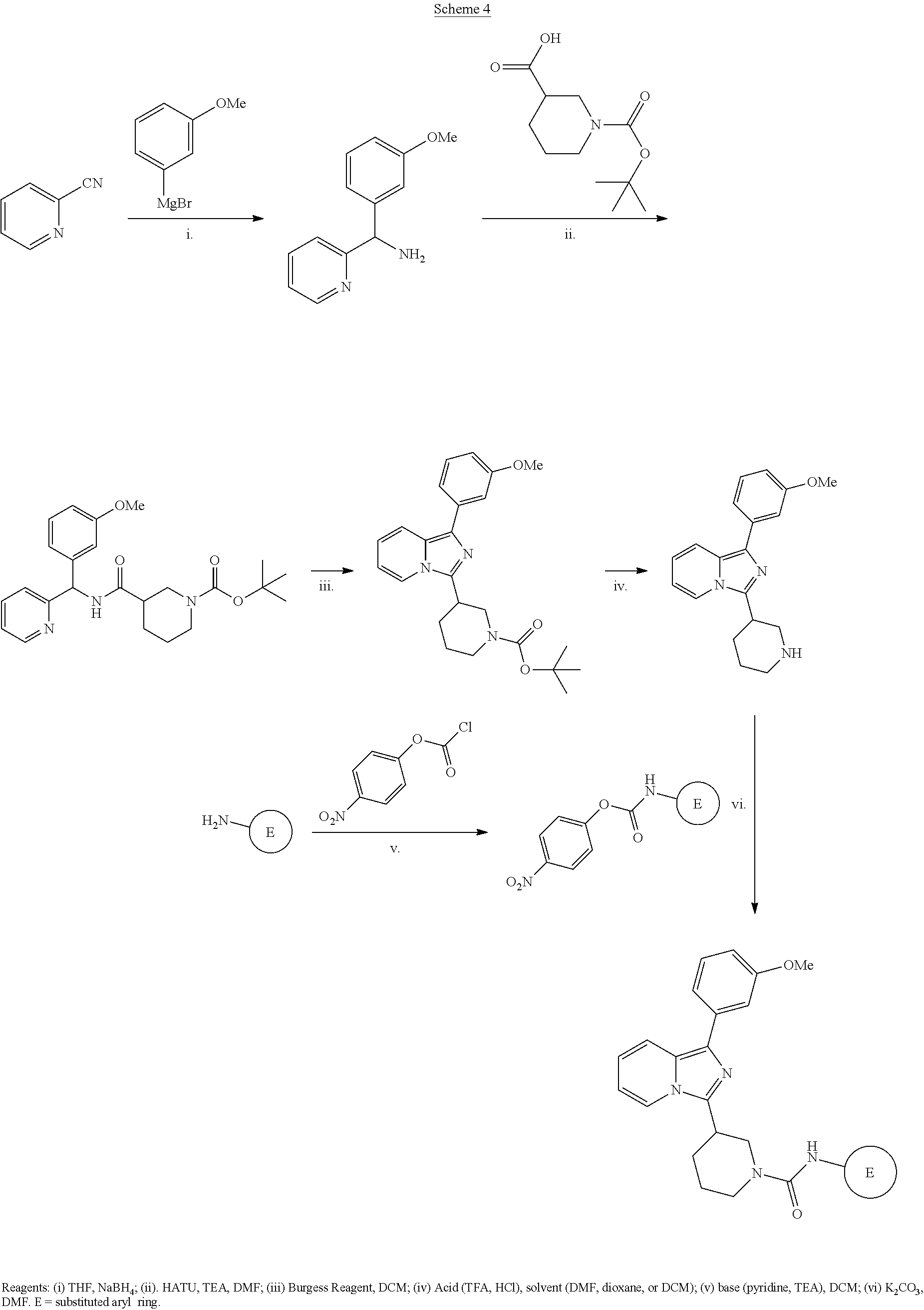

### Step 4-1. (3-methoxyphenyl)(pyridin-2-yl)methanamine (INT 4A)

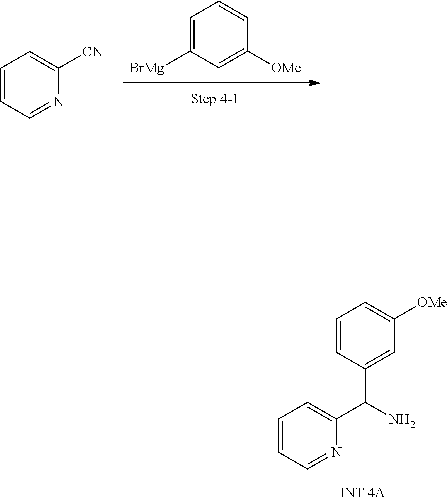

Into a cooled (0° C.) solution of pyridine-2-carbonitrile (3.0 g, 29 mmol) in dry THF (50 mL) under N₂ was added bromo-(3-methoxyphenyl)magnesium (1 M, 34.6 mL, 34.6 mmol) dropwise over 0.5 h. The reaction mixture was then stirred at 25° C. for 1.5 h under N₂ atmosphere. The reaction mixture was cooled back down to 0° C., and isobutanol (30 mL) was added dropwise, keeping the temperature below 5° C. The reaction mixture was kept cool (0-5° C.) and NaBH₄ (1.64 g, 43.2 mmol) was added portion wise. The reaction mixture was stirred at 25° C. for 12 h under N₂ atmosphere. The mixture was diluted with H₂O at 25° C. and stirred for 0.15 h, then filtered. The filter cake was dried in vacuo to provide 3.9 g (632%) of (3-methoxyphenyl)(pyridin-2-yl)methanamine_(INT 4A) as a white solid. LCMS-ESI (m/z) calculated for C₁₃H₁₄N₂O: 214.3; found 215.2 [M+H]⁺, t_{R}=0.67 min (Method 4). ¹H NMR (400 MHZ, DMSO-d6): δ=8.45 (d, J=4.6 Hz, 1H), 7.71 (dt, J=1.8, 7.6 Hz, 1H), 7.46 (d, J=7.9 Hz, 1H), 7.22-7.13 (m, 2H), 6.97 (d, J=1.9 Hz, 1H), 6.93 (d, J=7.6 Hz, 1H), 6.73 (dd, J=2.4, 8.1 Hz, 1H), 5.06 (t, J=6.4 Hz, 1H), 3.70 (s, 3H), 2.41 (br d, J=6.4 Hz, 2H).

### Step 4-2. Synthesis of tert-butyl 3-(((3-methoxyphenyl) (pyridin-2-yl)methyl) carbamoyl) piperidine-1-carboxylate (INT 4B)

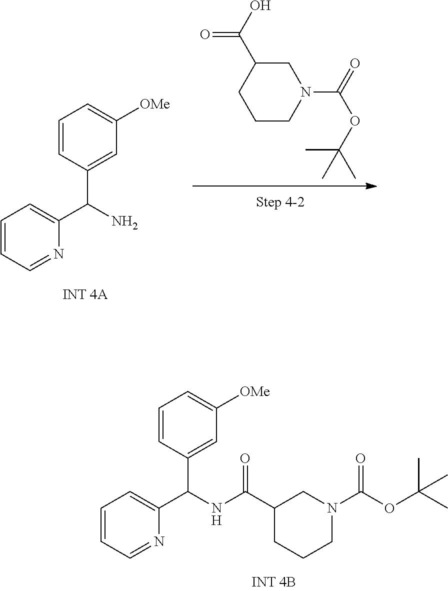 [SMILES: COc1cccc(C(N)c2ccccn2)c1 ; CC(C)(C)OC(=O)N1CCCC(C(=O)O)C1 ; COc1cccc(C(NC(=O)C2CCCN(C(=O)OC(C)(C)C)C2)c2ccccn2)c1]

Into a solution of 1-tert-butoxycarbonylpiperidine-3-carboxylic acid (5.46 g, 23.8 mmol) in DMF (50 mL) were added HATU (9.05 g, 23.8 mmol) and TEA (6.63 mL, 47.6 mmol). The reaction was stirred at 25° C. for 0.15 h under an N₂ atmosphere, before INT 4A (3.4 g, 16 mmol) was added. The mixture was then stirred at 60° C. for 12 h under N₂ atmosphere, quenched with H₂O (50 mL) and extracted with EA (3×). The combined organic layers were washed with saturated NaCl (aq), dried (Na₂SO₄), and concentrated under reduced pressure to provide 3.1 g (46%) of tert-butyl 3-(((3-methoxyphenyl) (pyridin-2-yl)methyl) carbamoyl)piperidine-1-carboxylate (INT 4B) that was used without further purification. LCMS-ESI (m/z) calculated for C₂₉H₃₁N₃O₄: 425.5; found 426.2 [M+H]⁺, t_{R}=0.863 min (Method 4). ¹H NMR (400 MHZ, DMSO-d6): δ=8.86 (br d, J=8.4 Hz, 1H), 8.57-8.47 (m, 1H), 7.77 (tt, J=1.8, 7.7 Hz, 1H), 7.44 (d, J=7.9 Hz, 1H), 7.30-7.18 (m, 2H), 6.92-6.85 (m, 2H), 6.79 (ddd, J=2.2, 5.4, 7.8 Hz, 1H), 6.09 (dd, J=5.6, 8.3 Hz, 1H), 3.71 (d, J=3.0 Hz, 3H), 3.49-3.42 (m, 2H), 3.36-3.13 (m, 2H), 2.64-2.58 (m, 1H), 1.74 (br d, J=14.1 Hz, 1H), 1.55-1.46 (m, 2H), 1.39 (s, 10H), 1.32-1.25 (m, 1H).

### Step 4-3. Synthesis of tert-butyl 3-(1-(3-methoxyphenyl)imidazo[1,5-a]pyridin-3-yl)piperidine-1-carboxylate (INT 3D)

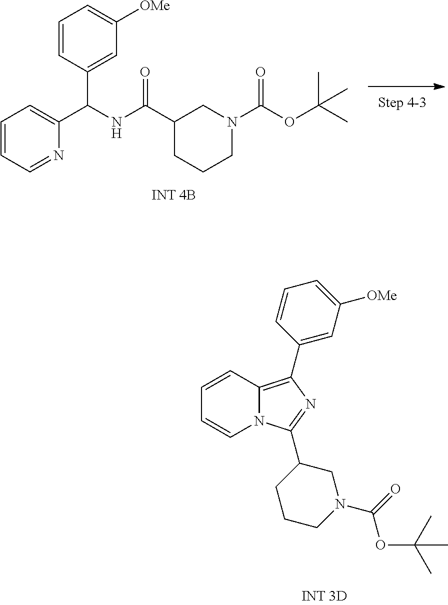 [SMILES: COc1cccc(C(NC(=O)C2CCCN(C(=O)OC(C)(C)C)C2)c2ccccn2)c1 ; COc1cccc(-c2nc(C3CCCN(C(=O)OC(C)(C)C)C3)n3ccccc23)c1]

To a solution of INT 4B (3 g, 7.1 mmol) in DCM (40 mL) was added Burgess Reagent (1.85 g, 7.8 mmol) in H₂O (1 mL). After stirring at 25° C. for 16 h under an N₂ atmosphere, the reaction mixture was poured into H₂O and extracted with EA (3×). The combined organic layers were washed with brine, dried (Na₂SO₄), filtered, and concentrated to provide crude material that was purified by SiO₂ chromatography (EA/petroleum ether) to provide 1.27 g (44%) of tert-butyl 3-(1-(3-methoxyphenyl) imidazo[1,5-a]pyridin-3-yl)piperidine-1-carboxylate (INT 3D) as a green oil. LCMS-ESI (m/z) calculated for C₂₄H₂₉N₃O₃: 407.5; found 408.3 [M+H]*, t_{R}=0.92 min (Method 4). ¹H NMR (400 MHZ, CDCl₃-d) δ=7.89 (br s, 1H), 7.78 (br d, J=9.3 Hz, 1H), 7.46-7.39 (m, 2H), 7.37-7.31 (m, 1H), 6.88-6.80 (m, 1H), 6.78-6.71 (m, 1H), 6.65-6.54 (m, 1H), 4.52-4.15 (m, 2H), 3.90 (s, 3H), 3.30-3.03 (m, 2H), 3.02-2.68 (m, 2H), 2.23-2.12 (m, 1H), 1.97-1.82 (m, 1H), 1.65-1.56 (m, 1H), 1.55-1.48 (m, 9H).

### Step 4-4. Synthesis of 1-(3-methoxyphenyl)-3-(piperidin-3-yl)imidazo[1,5-a]pyridine (INT 3E)

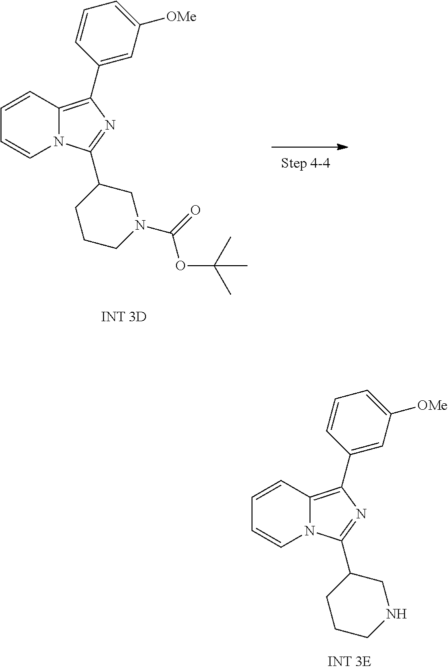 [SMILES: COc1cccc(-c2nc(C3CCCN(C(=O)OC(C)(C)C)C3)n3ccccc23)c1 ; COc1cccc(-c2nc(C3CCCNC3)n3ccccc23)c1]

A solution of INT 3D (1.27 g, 3.1 mmol) in 1M HCl/Dioxane (15 mL) was stirred at 25° C. for 1 h under an N₂ atmosphere. The reaction mixture was concentrated to provide 1.12 g (99% yield) of crude 1-(3-methoxyphenyl)-3-(piperidin-3-yl)imidazo[1,5-a]pyridine (INT 3E) as a yellow solid. LCMS-ESI (m/z) calculated for C₁₉H21lN₃O: 307.4; found 308.2 [M+H]⁺, t_{R}=0.76 min (Method 4). ¹H NMR (400 MHz, DMSO-d6): δ=9.43 (br s, 2H), 8.51 (br d, J=7.0 Hz, 1H), 8.04 (d, J=9.3 Hz, 1H), 7.57-7.42 (m, 3H), 7.12 (br dd, J=6.8, 8.9 Hz, 1H), 7.04-6.87 (m, 2H), 4.10-4.02 (m, 1H), 3.91 (s, 3H), 3.62-3.38 (m, 4H), 3.14-2.97 (m, 1H), 2.29-2.17 (m, 1H), 2.17-2.00 (m, 2H).

### Step 4-5. Synthesis of 4-nitrophenyl (3,4,5-trimethoxyphenyl)carbamate (INT 4C)

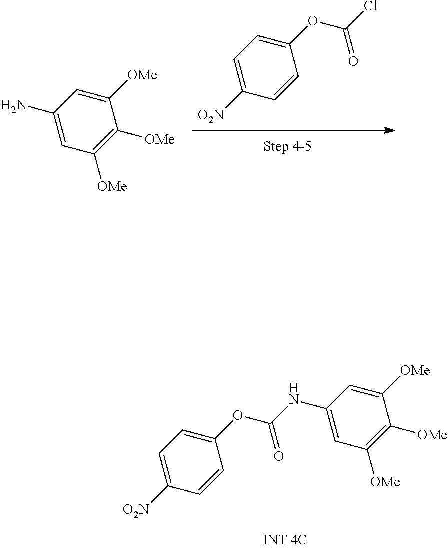 [SMILES: COc1cc(N)cc(OC)c1OC ; O=C(Cl)Oc1ccc([N+](=O)[O-])cc1 ; COc1cc(NC(=O)Oc2ccc([N+](=O)[O-])cc2)cc(OC)c1OC]

To a solution of 3,4,5-trimethoxyaniline (1.0 g, 5.5 mmol) in DCM (15 mL) and pyridine (5 mL) was added (4-nitrophenyl) carbonochloridate (1.1 g, 5.5 mmol) portion wise, with stirring over 30 using ice-bath cooling. After stirring at 25° C. for 1 h, the reaction mixture was concentrated and purified by SiO₂ chromatography (EA/petroleum ether) to provide 450 mg (24%) of 4-nitrophenyl (3,4,5-trimethoxyphenyl)carbamate (INT 4C) as a yellow solid. LCMS-ESI (m/z) calculated for C₁₆H₁₆N₂O₇: 348.3; m/z not observed, t_{R}=0.89 min (Method 4). NMR (400 MHZ, CDCl₃-d): δ=8.35-8.26 (m, 2H), 7.48-7.34 (m, 2H), 7.01 (br s, 1H), 6.75 (s, 2H), 3.87 (s, 6H), 3.84 (s, 3H).

### Step 4-6. Synthesis of 3-(1-(3-methoxyphenyl)imidazo[1,5-a]pyridin-3-yl)-N-(3,4,5-trimethoxyphenyl)piperidine-1-carboxamide (Compound 4-2)

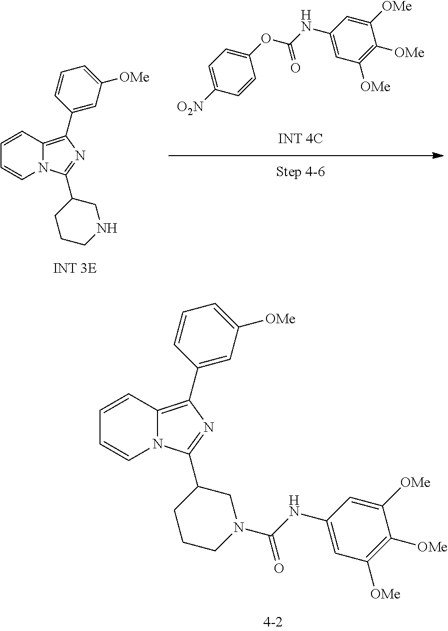 [SMILES: COc1cccc(-c2nc(C3CCCNC3)n3ccccc23)c1 ; COc1cc(NC(=O)Oc2ccc([N+](=O)[O-])cc2)cc(OC)c1OC ; COc1cccc(-c2nc(C3CCCN(C(=O)Nc4cc(OC)c(OC)c(OC)c4)C3)n3ccccc23)c1]

To a solution of INT 3E (80 mg, 0.23 mmol, made by either scheme 1 or scheme 4) and INT 4C (122 mg, 0.35 mmol) in DMF (1.5 mL) was added K₂CO₃ (64 mg, 0.47 mmol). After stirring at 60° C. for 0.5 h, the reaction mixture was concentrated and the crude residue was purified by prep HPLC chromatography to provide 31.3 mg (28%) of 3-(1-(3-methoxyphenyl) imidazo[1,5-a]pyridin-3-yl)-N-(3,4,5-trimethoxyphenyl) piperidine-1-carboxamide (Compound 4-2) as a yellow solid. LCMS-ESI (m/z) calculated for C₂₉H₃₂N₄O₅: 516.6; found 517.2 [M+H]⁺, t_{R}=1.07 min (Method 9). ¹H NMR (400 MHZ, CD₄OD) J=8.25 (d, J=7.4 Hz, 1H), 7.81 (d, J=9.4 Hz, 1H), 7.38 (d, J=4.8 Hz, 3H), 6.92-6.85 (m, 2H), 6.80-6.68 (m, 3H), 4.64 (s, 3H), 4.37 (br d, J=9.6 Hz, 1H), 4.16 (br d, J=13.9 Hz, 1H), 3.82-3.76 (m, 1H), 3.86-3.72 (m, 9H), 3.75-3.71 (m, 1H), 3.90-3.70 (m, 1H), 3.53-3.43 (m, 2H), 3.28-3.16 (m, 1H), 2.32-2.06 (m, 2H), 2.02-1.72 (m, 2H).

The compounds listed in Table 4 were made using the procedures of Scheme 4.

TABLE 4

| | | Purity | | | | |
|---|---|---|---|---|---|---|
| | Cpd | RT | | Observed | | Purity |
| Structure | No. | (min) | MW | m/z | Ion | Method |
| 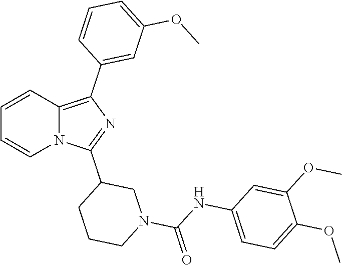 [SMILES: COc1cccc(-c2nc(C3CCCN(C(=O)Nc4ccc(OC)c(OC)c4)C3)n3ccccc23)c1] | 4-1 | 1.047 | 486.2 | 487.2 | [M + H]⁺ | 9 |
| 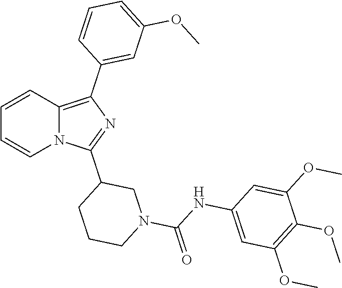 [SMILES: COc1cccc(-c2nc(C3CCCN(C(=O)Nc4cc(OC)c(OC)c(OC)c4)C3)n3ccccc23)c1] | 4-2 | 1.068 | 516.2 | 517.2 | [M + H]⁺ | 9 |
| 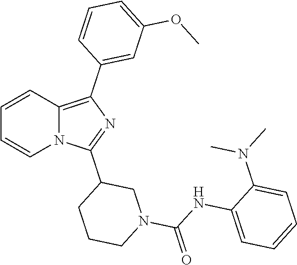 [SMILES: COc1cccc(-c2nc(C3CCCN(C(=O)Nc4ccccc4N(C)C)C3)n3ccccc23)c1] | 4-3 | 0.791 | 469.2 | 470.3 | [M + H]⁺ | 4 |
| 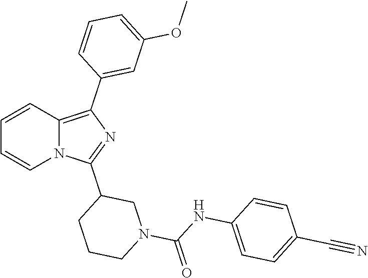 [SMILES: COc1cccc(-c2nc(C3CCCN(C(=O)Nc4ccc(C#N)cc4)C3)n3ccccc23)c1] | 4-4 | 0.805 | 451.2 | 452.2 | [M + H]⁺ | 9 |

### Example 5

### Synthesis of Compound 5-4 and Other Representative Compounds

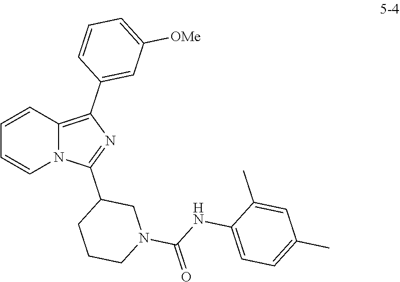 [SMILES: COc1cccc(-c2nc(C3CCCN(C(=O)Nc4ccc(C)cc4C)C3)n3ccccc23)c1]

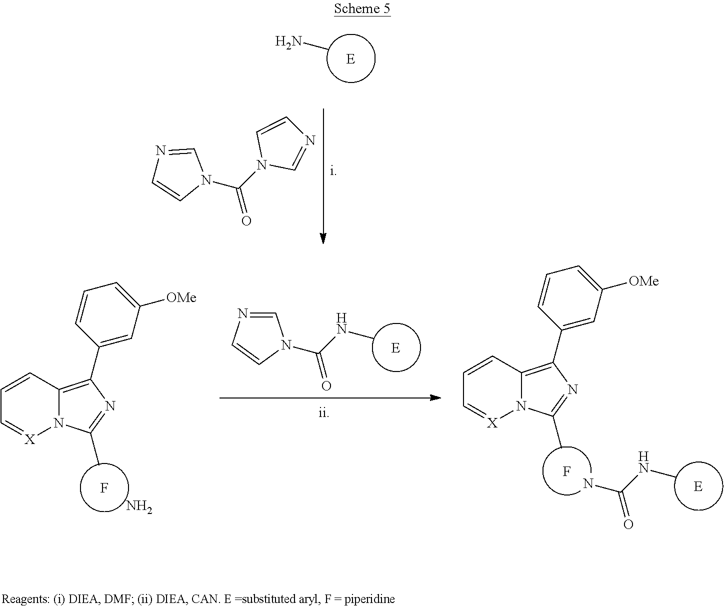

### Step 5-1. Synthesis of N-(2,4-dimethylphenyl)-1H-imidazole-1-carboxamide (INT 5A)

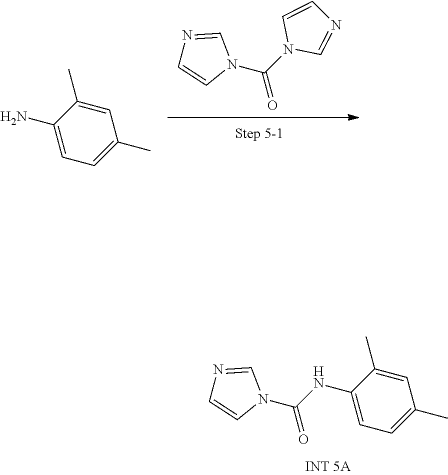

A solution of 2,4-dimethylaniline (100 mg, 0.82 mmol) in THF (1 mL) was treated with DIEA (287.5 uL, 1.65 mmol) and then carbonyl di-imidazole (161 mg, 0.99 mmol). The reaction mixture was stirred at 25° C. for 2 h and then concentrated to provide 150 mg (99%) of crude N-(2,4-dimethylphenyl)-1H-imidazole-1-carboxamide (INT 5A) as a pale-yellow solid that was used without further purification. LCMS-ESI (m/z) calculated for C₇H₆N₄O: 162.2; m/z not observed, t_{R}=0.67 min (Method 4).

### Step 5-2. Synthesis of N-(2,4-dimethylphenyl)-3-(1-(3-methoxyphenyl)imidazo[1,5-a]pyridin-3-yl)piperidine-1-carboxamide (Compound 5-4)

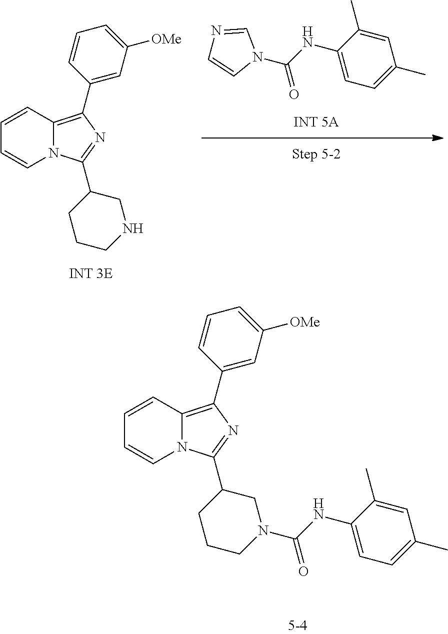 [SMILES: COc1cccc(-c2nc(C3CCCNC3)n3ccccc23)c1 ; Cc1ccc(NC(=O)n2ccnc2)c(C)c1 ; COc1cccc(-c2nc(C3CCCN(C(=O)Nc4ccc(C)cc4C)C3)n3ccccc23)c1]

Into a solution of INT 5A (84 mg, 0.39 mmol) and DIEA (90.7 μL, 0.39 mmol) in ACN (2 ml) was added INT 3E (80 mg, 0.26 mmol). After stirring at 80° C. for 3 h, the reaction mixture was concentrated and purified by RP-SiO₂ chromatography (H₂O (10 mM NH₄CO₃)/ACN) to afford 47 mg (40%) of N-(2,4-dimethylphenyl)-3-(1-(3-methoxyphenyl)imidazo[1,5-a]pyridin-3-yl)piperidine-1-carboxamide (Compound 5-4) as a pale yellow solid. LCMS-ESI (m/z) calculated for C₂₈H₃₀N₄O₂: 454.6; found 455.3 [M+H]⁺, t_{R}=0.82 min (Method 4) ¹H NMR (400 MHZ, CDCl₃) δ=1.72-1.85 (m, 1H), 1.86-1.96 (m, 1H), 2.22 (s, 3H), 2.30 (s, 5H), 3.25-3.37 (m, 3H), 3.86 (s, 4H), 4.38 (br d, J=9.41 Hz, 1H), 6.51 (s, 1H), 6.58 (t, J=6.72 Hz, 1H), 6.75 (dd, J=9.17, 6.36 Hz, 1H), 6.80-6.86 (m, 1H), 6.97-7.04 (m, 2H), 7.31-7.37 (m, 1H), 7.39-7.49 (m, 3H), 7.78 (d, J=9.29 Hz, 1H), 7.95 (d, J=7.21 Hz, 1H).

The compounds listed in Table 5 were made using the procedures of Scheme 5.

TABLE 5

| | | Purity | | | | |
|---|---|---|---|---|---|---|
| | Cpd | RT | | Observed | | Purity |
| Structure | No. | (min) | MW | m/z | Ion | Method |
| 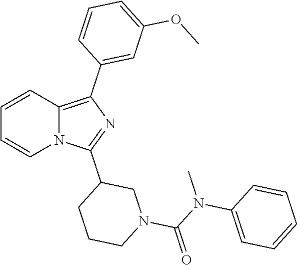 [SMILES: COc1cccc(-c2nc(C3CCCN(C(=O)N(C)c4ccccc4)C3)n3ccccc23)c1] | 5-1 | 0.913 | 440.2 | 441.3 | [M + H]⁺ | 4 |
| 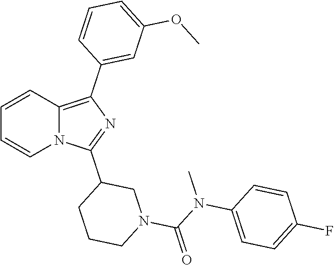 [SMILES: COc1cccc(-c2nc(C3CCCN(C(=O)N(C)c4ccc(F)cc4)C3)n3ccccc23)c1] | 5-2 | 0.919 | 458.2 | 459.2 | [M + H]⁺ | 4 |
| 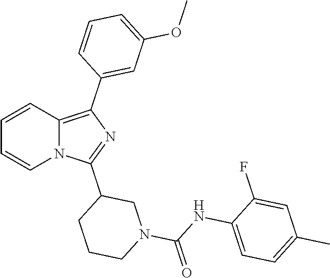 [SMILES: COc1cccc(-c2nc(C3CCCN(C(=O)Nc4ccc(C)cc4F)C3)n3ccccc23)c1] | 5-3 | 0.819 | 458.2 | 459.2 | [M + H]⁺ | 4 |
| 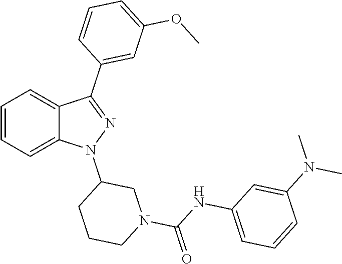 [SMILES: COc1cccc(-c2nn(C3CCCN(C(=O)Nc4cccc(N(C)C)c4)C3)c3ccccc23)c1] | 5-4 | 8.54 | 469.2 | 470 | [M + H]⁺ | 1 |
| 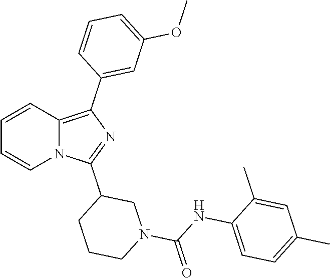 [SMILES: COc1cccc(-c2nc(C3CCCN(C(=O)Nc4ccc(C)cc4C)C3)n3ccccc23)c1] | 5-5 | 0.82 | 454.2 | 455.3 | [M + H]⁺ | 4 |
| 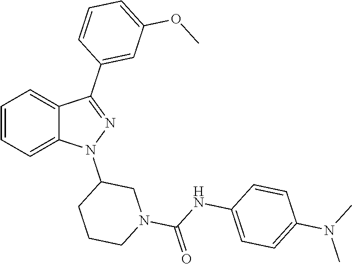 [SMILES: COc1cccc(-c2nn(C3CCCN(C(=O)Nc4ccc(N(C)C)cc4)C3)c3ccccc23)c1] | 5-6 | 7.099 | 469.2 | 470 | [M + H]⁺ | 1 |
| 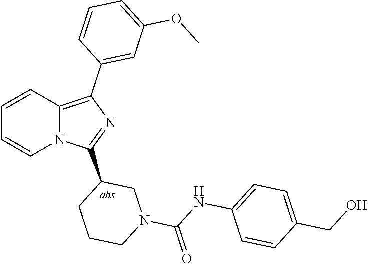 [SMILES: COc1cccc(-c2nc([C@@H]3CCCN(C(=O)Nc4ccc(CO)cc4)C3)n3ccccc23)c1] | 5-7 | 10.56 | 456.2 | 456.7 | [M + H]⁺ | 2 |
| 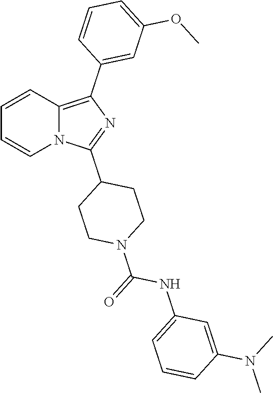 [SMILES: COc1cccc(-c2nc(C3CCN(C(=O)Nc4cccc(N(C)C)c4)CC3)n3ccccc23)c1] | 5-8 | 5.354 | 469.2 | 467.8 | [M + H]⁺ | 10 |
| 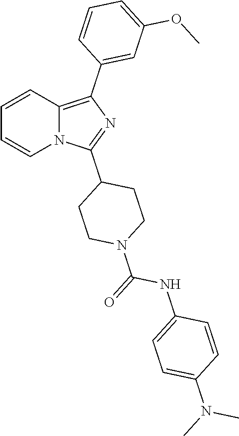 [SMILES: COc1cccc(-c2nc(C3CCN(C(=O)Nc4ccc(N(C)C)cc4)CC3)n3ccccc23)c1] | 5-9 | 4.76 | 469.2 | 470 | [M + H]⁺ | 1 |
| 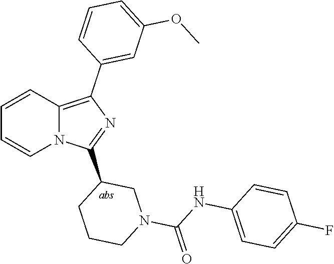 [SMILES: COc1cccc(-c2nc([C@@H]3CCCN(C(=O)Nc4ccc(F)cc4)C3)n3ccccc23)c1] | 5-10 | 11.71 | 444.2 | 444.9 | [M + H]⁺ | 2 |
| 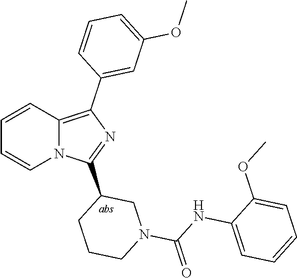 [SMILES: COc1cccc(-c2nc([C@@H]3CCCN(C(=O)Nc4ccccc4OC)C3)n3ccccc23)c1] | 5-11 | 11.46 | 456.2 | 456.9 | [M + H]⁺ | 2 |
| 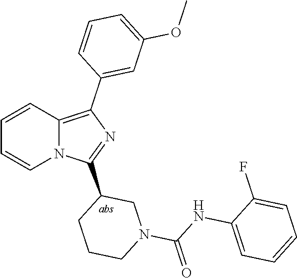 [SMILES: COc1cccc(-c2nc([C@@H]3CCCN(C(=O)Nc4ccccc4F)C3)n3ccccc23)c1] | 5-12 | 11.26 | 444.2 | 444.5 | [M + H]⁺ | 2 |
| 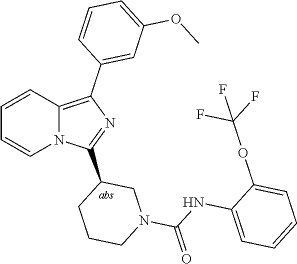 [SMILES: COc1cccc(-c2nc([C@@H]3CCCN(C(=O)Nc4ccccc4OC(F)(F)F)C3)n3ccccc23)c1] | 5-13 | 11.69 | 510.2 | 510.5 | [M + H]⁺ | 2 |
| 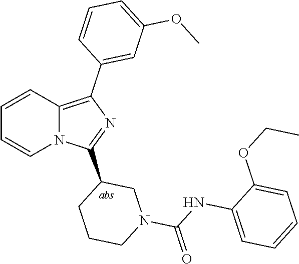 [SMILES: CCOc1ccccc1NC(=O)N1CCC[C@@H](c2nc(-c3cccc(OC)c3)c3ccccn23)C1] | 5-14 | 11.86 | 470.2 | 470.9 | [M + H]⁺ | 2 |
| 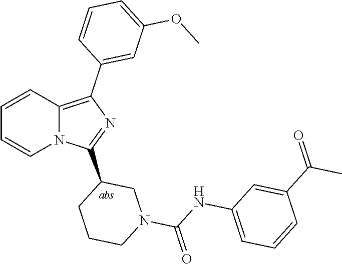 [SMILES: COc1cccc(-c2nc([C@@H]3CCCN(C(=O)Nc4cccc(C(C)=O)c4)C3)n3ccccc23)c1] | 5-15 | 10.06 | 468.2 | 468.6 | [M + H]⁺ | 2 |
| 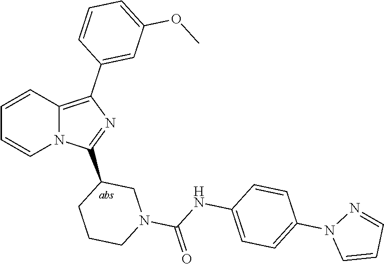 [SMILES: COc1cccc(-c2nc([C@@H]3CCCN(C(=O)Nc4ccc(-n5cccn5)cc4)C3)n3ccccc23)c1] | 5-16 | 11.44 | 492.2 | 492.8 | [M + H]⁺ | 2 |
| 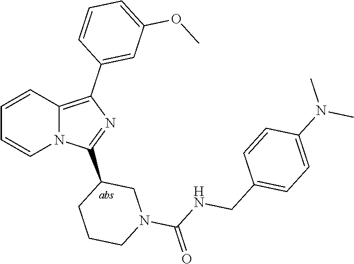 [SMILES: COc1cccc(-c2nc([C@@H]3CCCN(C(=O)NCc4ccc(N(C)C)cc4)C3)n3ccccc23)c1] | 5-17 | 9.47 | 483.3 | 484 | [M + H]⁺ | 2 |
| 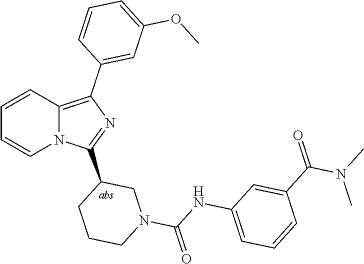 [SMILES: COc1cccc(-c2nc([C@@H]3CCCN(C(=O)Nc4cccc(C(=O)N(C)C)c4)C3)n3ccccc23)c1] | 5-18 | 10.75 | 497.2 | 497.7 | [M + H]⁺ | 2 |
| 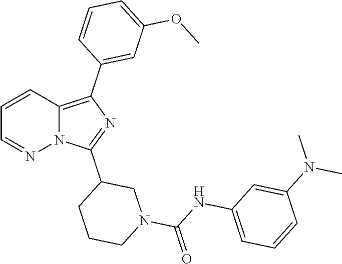 [SMILES: COc1cccc(-c2nc(C3CCCN(C(=O)Nc4cccc(N(C)C)c4)C3)n3ncccc23)c1] | 5-19 | 6.472 | 470.2 | 471 | [M + H]⁺ | 1 |
| 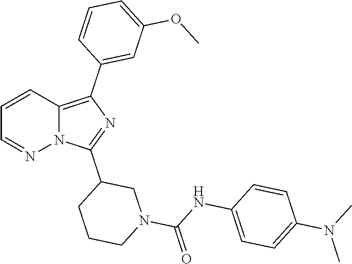 [SMILES: COc1cccc(-c2nc(C3CCCN(C(=O)Nc4ccc(N(C)C)cc4)C3)n3ncccc23)c1] | 5-20 | 5.612 | 470.2 | 471 | [M + H]⁺ | 1 |
| 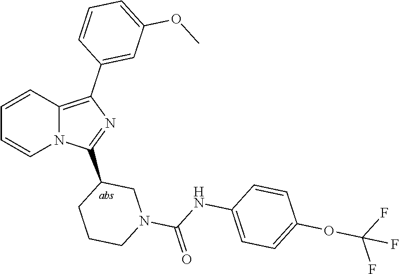 [SMILES: COc1cccc(-c2nc([C@@H]3CCCN(C(=O)Nc4ccc(OC(F)(F)F)cc4)C3)n3ccccc23)c1] | 5-21 | 12.35 | 510.2 | 511 | [M + H]⁺ | 2 |
| 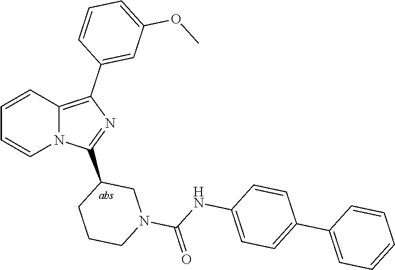 [SMILES: COc1cccc(-c2nc([C@@H]3CCCN(C(=O)Nc4ccc(-c5ccccc5)cc4)C3)n3ccccc23)c1] | 5-22 | 12.73 | 502.2 | 502.8 | [M + H]⁺ | 2 |
| 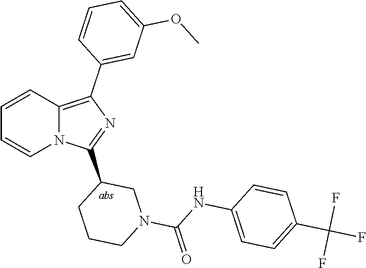 [SMILES: COc1cccc(-c2nc([C@@H]3CCCN(C(=O)Nc4ccc(C(F)(F)F)cc4)C3)n3ccccc23)c1] | 5-23 | 12.36 | 494.2 | 494.8 | [M + H]⁺ | 2 |
| 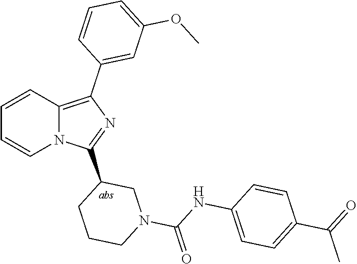 [SMILES: COc1cccc(-c2nc([C@@H]3CCCN(C(=O)Nc4ccc(C(C)=O)cc4)C3)n3ccccc23)c1] | 5-24 | 11.33 | 468.2 | 469.1 | [M + H]⁺ | 2 |
| 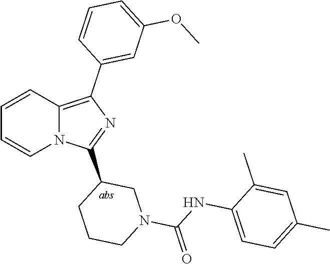 [SMILES: COc1cccc(-c2nc([C@@H]3CCCN(C(=O)Nc4ccc(C)cc4C)C3)n3ccccc23)c1] | 5-25 | 11.8 | 454.2 | 454.8 | [M + H]⁺ | 2 |
| 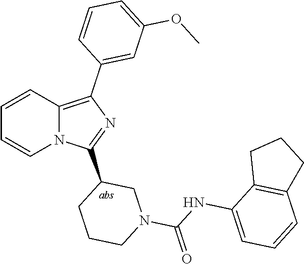 [SMILES: COc1cccc(-c2nc([C@@H]3CCCN(C(=O)Nc4cccc5c4CCC5)C3)n3ccccc23)c1] | 5-26 | 12.1 | 466.2 | 466.9 | [M + H]⁺ | 2 |
| 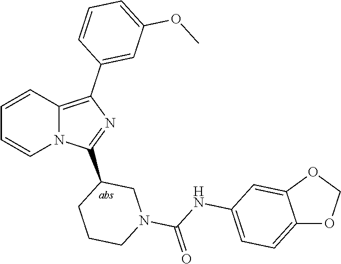 [SMILES: COc1cccc(-c2nc([C@@H]3CCCN(C(=O)Nc4ccc5c(c4)OCO5)C3)n3ccccc23)c1] | 5-27 | 11.26 | 470.2 | 470.7 | [M + H]⁺ | 2 |
| 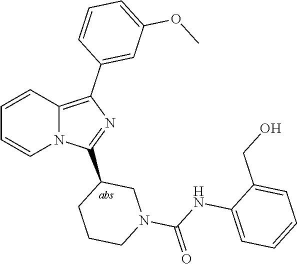 [SMILES: COc1cccc(-c2nc([C@@H]3CCCN(C(=O)Nc4ccccc4CO)C3)n3ccccc23)c1] | 5-28 | 10.16 | 456.2 | 456.5 | [M + H]⁺ | 2 |
| 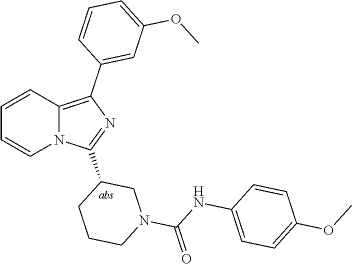 [SMILES: COc1ccc(NC(=O)N2CCC[C@H](c3nc(-c4cccc(OC)c4)c4ccccn34)C2)cc1] | 5-29 | 11.3 | 456.2 | 457.1 | [M + H]⁺ | 2 |
| 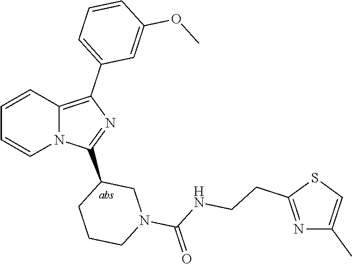 [SMILES: COc1cccc(-c2nc([C@@H]3CCCN(C(=O)NCCc4nc(C)cs4)C3)n3ccccc23)c1] | 5-30 | 10.45 | 475.2 | 475.7 | [M + H]⁺ | 2 |
| 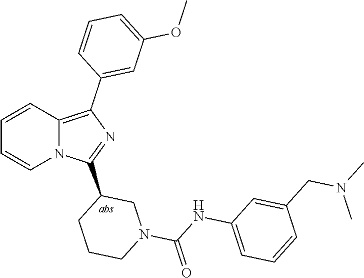 [SMILES: COc1cccc(-c2nc([C@@H]3CCCN(C(=O)Nc4cccc(CN(C)C)c4)C3)n3ccccc23)c1] | 5-31 | 10.04 | 483.3 | 484.1 | [M + H]⁺ | 2 |
| 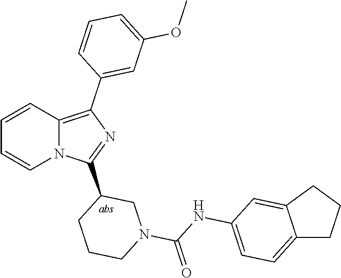 [SMILES: COc1cccc(-c2nc([C@@H]3CCCN(C(=O)Nc4ccc5c(c4)CCC5)C3)n3ccccc23)c1] | 5-32 | 12.57 | 466.2 | 466.9 | [M + H]⁺ | 2 |
| 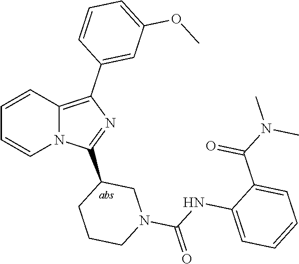 [SMILES: COc1cccc(-c2nc([C@@H]3CCCN(C(=O)Nc4ccccc4C(=O)N(C)C)C3)n3ccccc23)c1] | 5-33 | 11.05 | 497.2 | 497.9 | [M + H]⁺ | 2 |
| 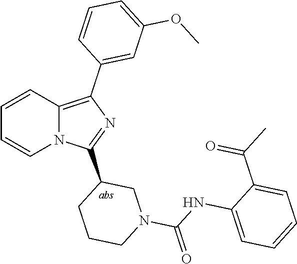 [SMILES: COc1cccc(-c2nc([C@@H]3CCCN(C(=O)Nc4ccccc4C(C)=O)C3)n3ccccc23)c1] | 5-34 | 12.55 | 468.2 | 468.7 | [M + H]⁺ | 2 |
| 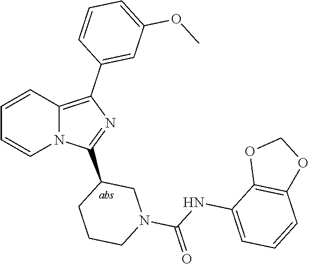 [SMILES: COc1cccc(-c2nc([C@@H]3CCCN(C(=O)Nc4cccc5c4OCO5)C3)n3ccccc23)c1] | 5-35 | 11.02 | 470.2 | 470.7 | [M + H]⁺ | 2 |
| 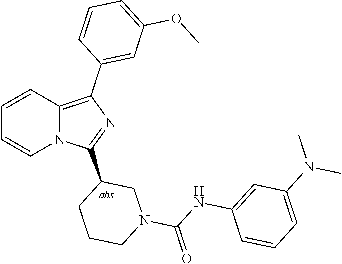 [SMILES: COc1cccc(-c2nc([C@@H]3CCCN(C(=O)Nc4cccc(N(C)C)c4)C3)n3ccccc23)c1] | 5-36 | 9.59 | 469.2 | 469.8 | [M + H]⁺ | 2 |
| 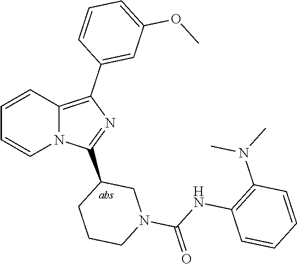 [SMILES: COc1cccc(-c2nc([C@@H]3CCCN(C(=O)Nc4ccccc4N(C)C)C3)n3ccccc23)c1] | 5-37 | 10.03 | 469.2 | 469.8 | [M + H]⁺ | 2 |
| 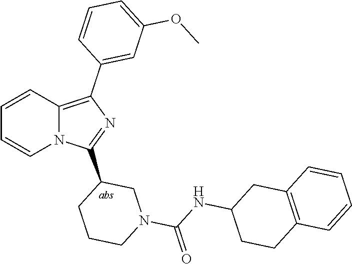 [SMILES: COc1cccc(-c2nc([C@@H]3CCCN(C(=O)NC4CCc5ccccc5C4)C3)n3ccccc23)c1] | 5-38 | 12.5 | 480.3 | 480.9 | [M + H]⁺ | 2 |
| 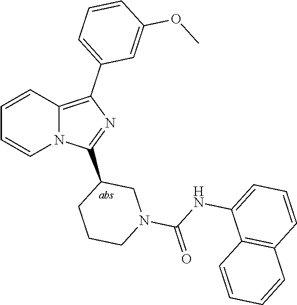 [SMILES: COc1cccc(-c2nc([C@@H]3CCCN(C(=O)Nc4cccc5ccccc45)C3)n3ccccc23)c1] | 5-39 | 12 | 476.2 | 476.6 | [M + H]⁺ | 2 |
| 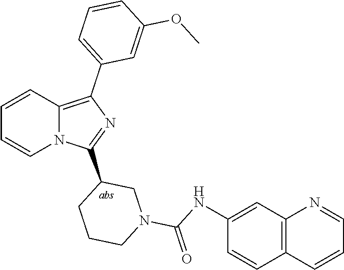 [SMILES: COc1cccc(-c2nc([C@@H]3CCCN(C(=O)Nc4ccc5cccnc5c4)C3)n3ccccc23)c1] | 5-40 | 10.47 | 477.2 | 477.8 | [M + H]⁺ | 2 |
| 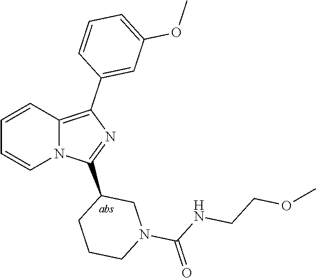 [SMILES: COCCNC(=O)N1CCC[C@@H](c2nc(-c3cccc(OC)c3)c3ccccn23)C1] | 5-41 | 9.96 | 408.2 | 408.6 | [M + H]⁺ | 2 |
| 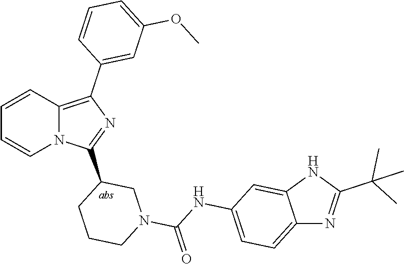 [SMILES: COc1cccc(-c2nc([C@@H]3CCCN(C(=O)Nc4ccc5nc(C(C)(C)C)[nH]c5c4)C3)n3ccccc23)c1] | 5-42 | 10.58 | 522.3 | 522.9 | [M + H]⁺ | 2 |
| 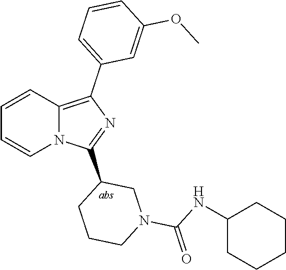 [SMILES: COc1cccc(-c2nc([C@@H]3CCCN(C(=O)NC4CCCCC4)C3)n3ccccc23)c1] | 5-43 | 12.25 | 432.3 | 432.7 | [M + H]⁺ | 2 |
| 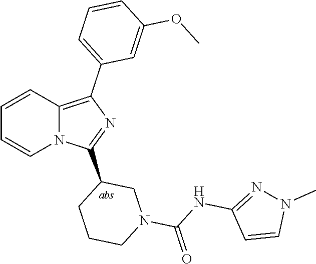 [SMILES: COc1cccc(-c2nc([C@@H]3CCCN(C(=O)Nc4ccn(C)n4)C3)n3ccccc23)c1] | 5-44 | 10.6 | 430.2 | 431 | [M + H]⁺ | 2 |
| 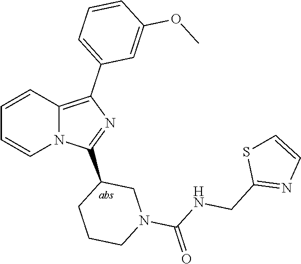 [SMILES: COc1cccc(-c2nc([C@@H]3CCCN(C(=O)NCc4nccs4)C3)n3ccccc23)c1] | 5-45 | 10.4 | 447.2 | 447.5 | [M + H]⁺ | 2 |
| 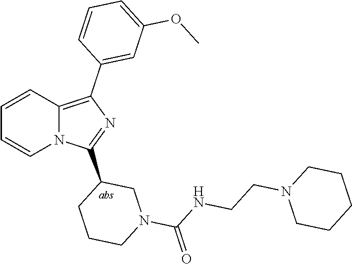 [SMILES: COc1cccc(-c2nc([C@@H]3CCCN(C(=O)NCCN4CCCCC4)C3)n3ccccc23)c1] | 5-46 | 10.08 | 461.3 | 461.9 | [M + H]⁺ | 2 |
| 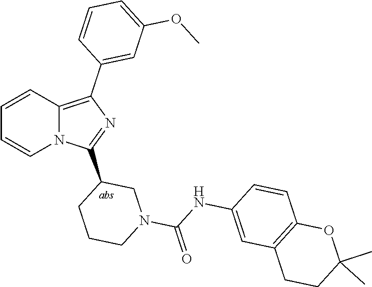 [SMILES: COc1cccc(-c2nc([C@@H]3CCCN(C(=O)Nc4ccc5c(c4)CCC(C)(C)O5)C3)n3ccccc23)c1] | 5-47 | 12.59 | 510.3 | 510.9 | [M + H]⁺ | 2 |
| 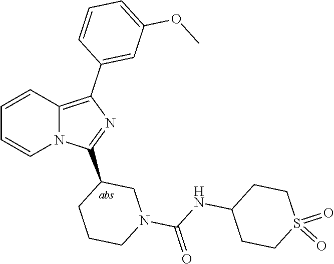 [SMILES: COc1cccc(-c2nc([C@@H]3CCCN(C(=O)NC4CCS(=O)(=O)CC4)C3)n3ccccc23)c1] | 5-48 | 10.09 | 482.2 | 482.8 | [M + H]⁺ | 2 |
| 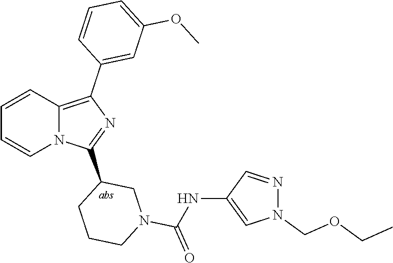 [SMILES: CCOCn1cc(NC(=O)N2CCC[C@@H](c3nc(-c4cccc(OC)c4)c4ccccn34)C2)cn1] | 5-49 | 10.62 | 474.2 | 474.5 | [M + H]⁺ | 2 |
| 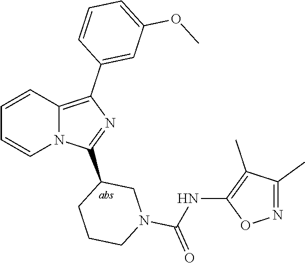 [SMILES: COc1cccc(-c2nc([C@@H]3CCCN(C(=O)Nc4onc(C)c4C)C3)n3ccccc23)c1] | 5-50 | 10.57 | 445.2 | 445.6 | [M + H]⁺ | 2 |
| 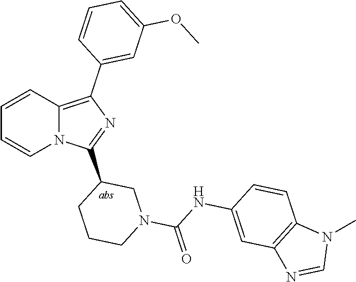 [SMILES: COc1cccc(-c2nc([C@@H]3CCCN(C(=O)Nc4ccc5c(c4)ncn5C)C3)n3ccccc23)c1] | 5-51 | 10.2 | 480.2 | 480.7 | [M + H]⁺ | 2 |
| 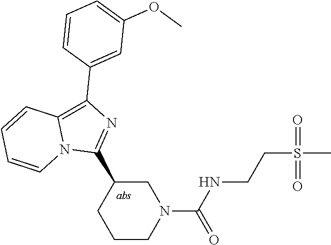 [SMILES: COc1cccc(-c2nc([C@@H]3CCCN(C(=O)NCCS(C)(=O)=O)C3)n3ccccc23)c1] | 5-52 | 9.4 | 456.2 | 457 | [M + H]⁺ | 2 |
| 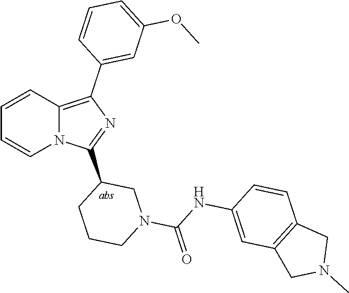 [SMILES: COc1cccc(-c2nc([C@@H]3CCCN(C(=O)Nc4ccc5c(c4)CN(C)C5)C3)n3ccccc23)c1] | 5-53 | 9.48 | 481.2 | 481.9 | [M + H]⁺ | 2 |
| 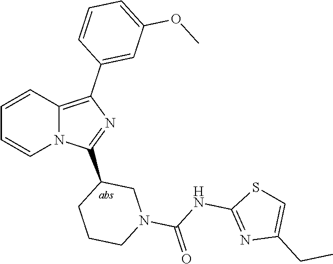 [SMILES: CCc1csc(NC(=O)N2CCC[C@@H](c3nc(-c4cccc(OC)c4)c4ccccn34)C2)n1] | 5-54 | 11.62 | 461.2 | 461.8 | [M + H]⁺ | 2 |
| 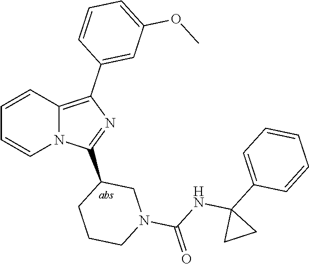 [SMILES: COc1cccc(-c2nc([C@@H]3CCCN(C(=O)NC4(c5ccccc5)CC4)C3)n3ccccc23)c1] | 5-55 | 11.61 | 466.2 | 466.8 | [M + H]⁺ | 2 |
| 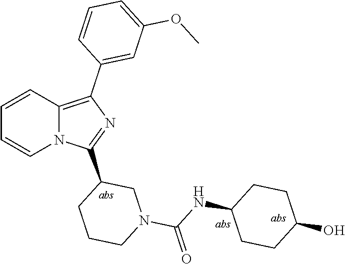 [SMILES: COc1cccc(-c2nc([C@@H]3CCCN(C(=O)N[C@H]4CC[C@@H](O)CC4)C3)n3ccccc23)c1] | 5-56 | 10.73 | 448.2 | 448.9 | [M + H]⁺ | 2 |
| 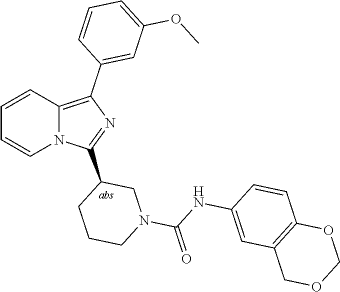 [SMILES: COc1cccc(-c2nc([C@@H]3CCCN(C(=O)Nc4ccc5c(c4)COCO5)C3)n3ccccc23)c1] | 5-57 | 11.39 | 484.2 | 485 | [M + H]⁺ | 2 |
| 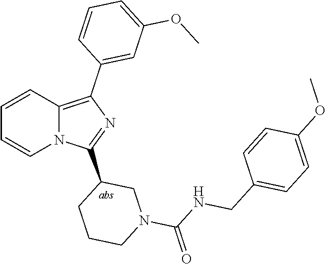 [SMILES: COc1ccc(CNC(=O)N2CCC[C@@H](c3nc(-c4cccc(OC)c4)c4ccccn34)C2)cc1] | 5-58 | 11.36 | 470.2 | 470.7 | [M + H]⁺ | 2 |
| 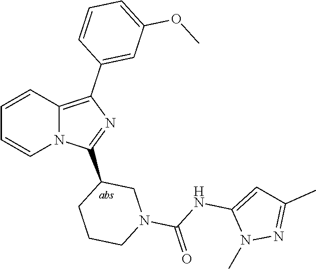 [SMILES: COc1cccc(-c2nc([C@@H]3CCCN(C(=O)Nc4cc(C)nn4C)C3)n3ccccc23)c1] | 5-59 | 10.24 | 444.2 | 444.6 | [M + H]⁺ | 2 |
| 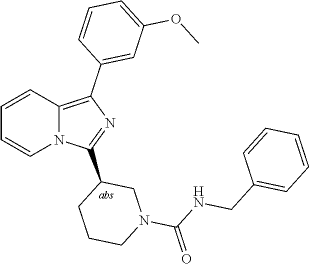 [SMILES: COc1cccc(-c2nc([C@@H]3CCCN(C(=O)NCc4ccccc4)C3)n3ccccc23)c1] | 5-60 | 11.64 | 440.2 | 441.1 | [M + H]⁺ | 2 |
| 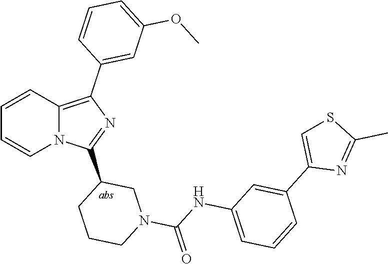 [SMILES: COc1cccc(-c2nc([C@@H]3CCCN(C(=O)Nc4cccc(-c5csc(C)n5)c4)C3)n3ccccc23)c1] | 5-61 | 12.35 | 523.2 | 523.8 | [M + H]⁺ | 2 |
| 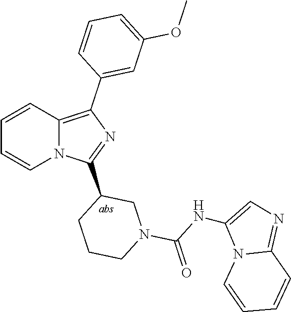 [SMILES: COc1cccc(-c2nc([C@@H]3CCCN(C(=O)Nc4cnc5ccccn45)C3)n3ccccc23)c1] | 5-62 | 9.65 | 466.2 | 466.9 | [M + H]⁺ | 2 |
| 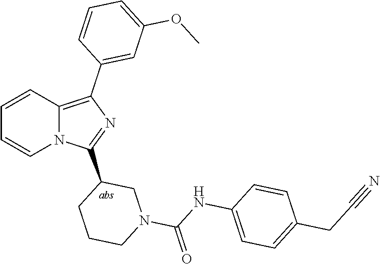 [SMILES: COc1cccc(-c2nc([C@@H]3CCCN(C(=O)Nc4ccc(CC#N)cc4)C3)n3ccccc23)c1] | 5-63 | 10.86 | 465.2 | 465.8 | [M + H]⁺ | 2 |
| 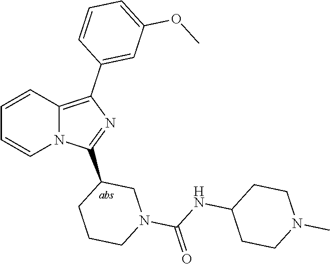 [SMILES: COc1cccc(-c2nc([C@@H]3CCCN(C(=O)NC4CCN(C)CC4)C3)n3ccccc23)c1] | 5-64 | 9.83 | 447.3 | 448 | [M + H]⁺ | 2 |
| 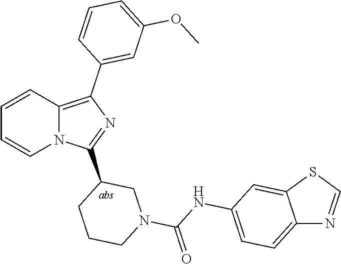 [SMILES: COc1cccc(-c2nc([C@@H]3CCCN(C(=O)Nc4ccc5ncsc5c4)C3)n3ccccc23)c1] | 5-65 | 11.55 | 483.2 | 483.5 | [M + H]⁺ | 2 |
| 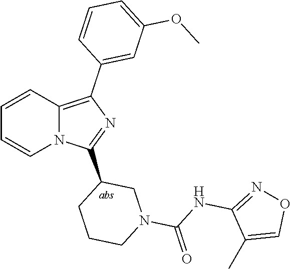 [SMILES: COc1cccc(-c2nc([C@@H]3CCCN(C(=O)Nc4nocc4C)C3)n3ccccc23)c1] | 5-66 | 10.5 | 431.2 | 431.8 | [M + H]⁺ | 2 |
| 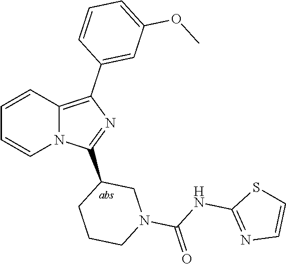 [SMILES: COc1cccc(-c2nc([C@@H]3CCCN(C(=O)Nc4nccs4)C3)n3ccccc23)c1] | 5-67 | 10.89 | 433.2 | 433.7 | [M + H]⁺ | 2 |
| 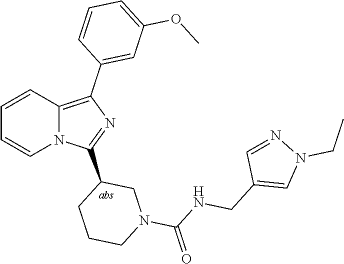 [SMILES: CCn1cc(CNC(=O)N2CCC[C@@H](c3nc(-c4cccc(OC)c4)c4ccccn34)C2)cn1] | 5-68 | 10.16 | 458.2 | 458.8 | [M + H]⁺ | 2 |
| 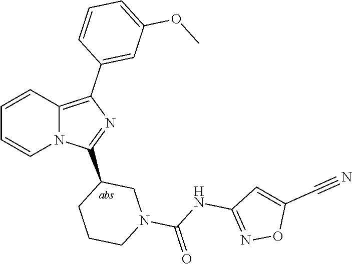 [SMILES: COc1cccc(-c2nc([C@@H]3CCCN(C(=O)Nc4cc(C#N)on4)C3)n3ccccc23)c1] | 5-69 | 8.96 | 442.2 | 442.9 | [M + H]⁺ | 2 |
| 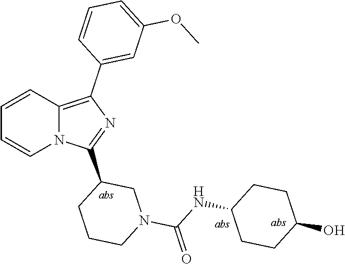 [SMILES: COc1cccc(-c2nc([C@@H]3CCCN(C(=O)N[C@H]4CC[C@H](O)CC4)C3)n3ccccc23)c1] | 5-70 | 8.38 | 448.2 | 448.7 | [M + H]⁺ | 2 |
| 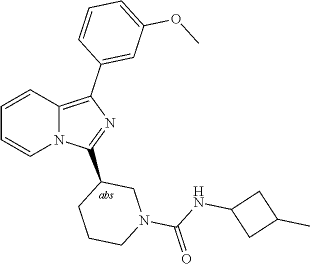 [SMILES: COc1cccc(-c2nc([C@@H]3CCCN(C(=O)NC4CC(C)C4)C3)n3ccccc23)c1] | 5-71 | 11.57 | 418.2 | 418.9 | [M + H]⁺ | 2 |
| 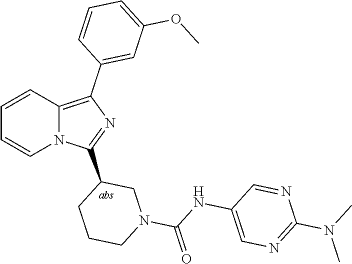 [SMILES: COc1cccc(-c2nc([C@@H]3CCCN(C(=O)Nc4cnc(N(C)C)nc4)C3)n3ccccc23)c1] | 5-72 | 10.5 | 471.2 | 471.8 | [M + H]⁺ | 2 |
| 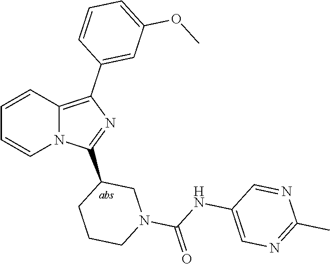 [SMILES: COc1cccc(-c2nc([C@@H]3CCCN(C(=O)Nc4cnc(C)nc4)C3)n3ccccc23)c1] | 5-73 | 10.47 | 442.2 | 443.1 | [M + H]⁺ | 2 |

### Example 6

### Synthesis of Compound 6-1 and Other Representative Compounds

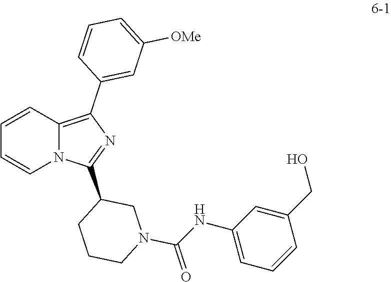 [SMILES: COc1cccc(-c2nc([C@@H]3CCCN(C(=O)Nc4cccc(CO)c4)C3)n3ccccc23)c1]

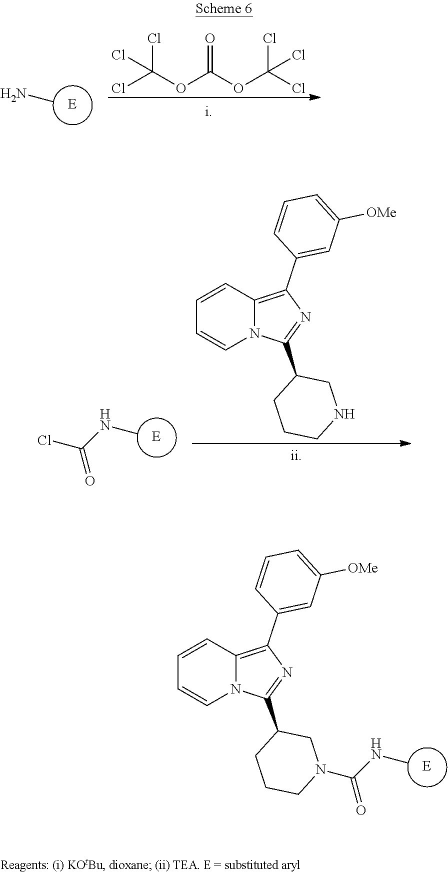

### Step 6-1. Synthesis of (R)—N-(3-(hydroxymethyl)phenyl)-3-(1-(3-methoxyphenyl) imidazo[1,5-a]pyridin-3-yl)piperidine-1-carboxamide (Compound 6-1)

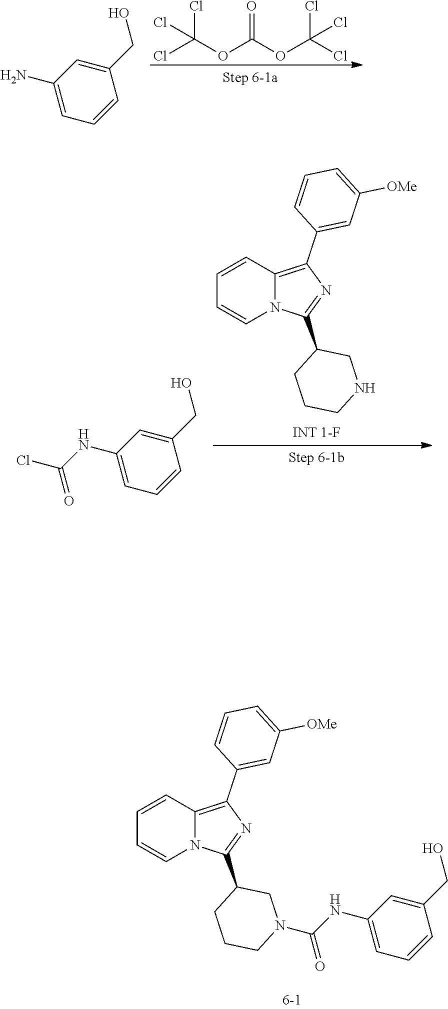

To a stirring solution of (3-aminophenyl)methanol (50 mg, 0.41 mmol) in dioxane (2.0 mL) was added KO^{t}Bu (50.1 mg, 0.45 mmol). After 5 min, bis(trichloromethyl) carbonate (66 mg, 0.41 mmol) was added. After stirring for 2 h, TEA (123 mg, 1.22 mmol) and INT 1F (50.1 mg, 0.15 mmol) were added. After stirring for 1 h at room temperature, the mixture was directly loaded onto a SiO₂ column and purified using SiO₂ chromatography (10% MeOH in EA/DCM). The resulting material was further purified by C-18 reverse-phase chromatography (MeOH/H₂O) to provide 21.5 mg (32% yield) of (R)—N-(3-(hydroxymethyl)phenyl)-3-(1-(3-methoxyphenyl) imidazo[1,5-a]pyridin-3-yl)piperidine-1-carboxamide (Compound 6-1). LCMS-ESI (m/z) calculated for C₂₇H₂₈N₄O₃: 456.5; found 457.7 [M+H]⁺, t_{R}=10.76 min (Method 2). ¹H NMR (400 MHz, DMSO): δ 8.58 (s, 1H), 8.35 (d, J=7.2 Hz, 1H), 7.90 (dd, J=1.2, 9.4 Hz, 1H), 7.50-7.31 (m, 5H), 7.16 (t, J=7.8 Hz, 1H), 6.89 (dd, J=6.7, 9.8 Hz, 2H), 6.85-6.79 (m, 1H), 6.75 (td, J=1.1, 6.2, 6.7 Hz, 1H), 5.13 (t, J=5.7 Hz, 1H), 4.44 (d, J=5.7 Hz, 2H), 4.38 (d, J=13.2 Hz, 1H), 4.24 (d, J=13.3 Hz, 1H), 3.82 (s, 3H), 3.29-3.18 (m, 1H), 2.96 (t, J=12.4 Hz, 1H), 2.16 (d, J=12.6 Hz, 1H), 1.83 (dd, J=11.6, 18.4 Hz, 2H), 1.68 (s, 1H).

The compounds listed in Table 6 were made using the procedures of Scheme 6.

TABLE 6

| | | Purity | | | | |
|---|---|---|---|---|---|---|
| | Cpd | RT | | Observed | | Purity |
| Structure | No. | (min) | MW | m/z | Ion | Method |
| 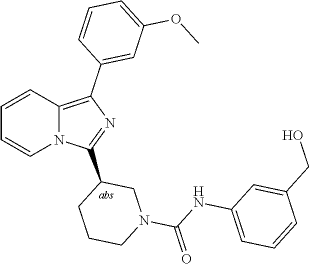 [SMILES: COc1cccc(-c2nc([C@@H]3CCCN(C(=O)Nc4cccc(CO)c4)C3)n3ccccc23)c1] | 6-1 | 10.8 | 456.2 | 456.8 | [M + H]⁺ | 2 |

### Example 7

### Synthesis of Compound 7-12 and Other Representative Compounds

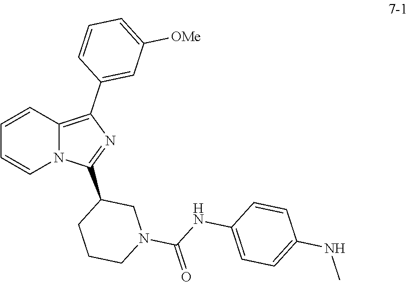 [SMILES: CNc1ccc(NC(=O)N2CCC[C@@H](c3nc(-c4cccc(OC)c4)c4ccccn34)C2)cc1]

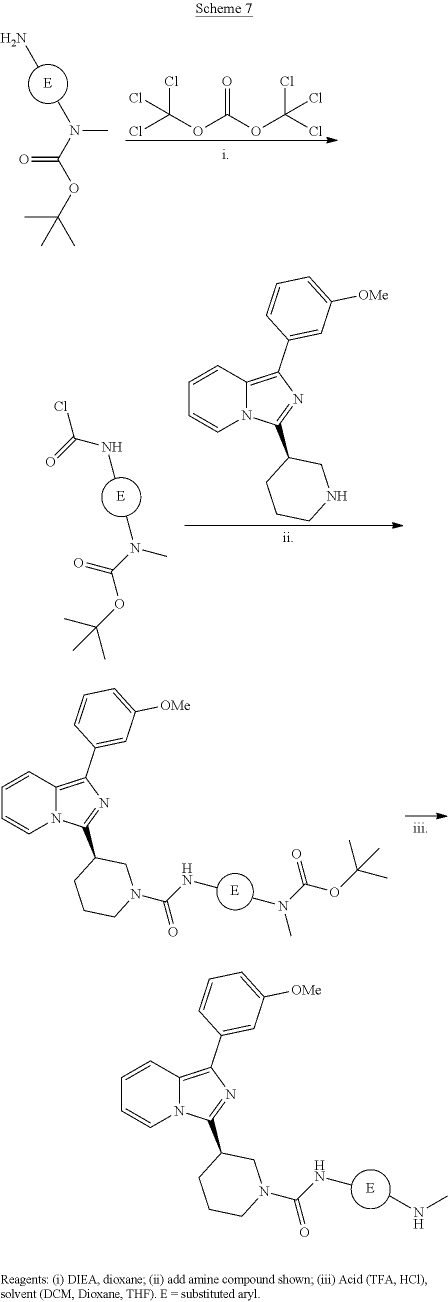

### Step 7-1. Synthesis of tert-butyl (R)-(4-(3-(1-(3-methoxyphenyl)imidazo[1,5-a]pyridin-3-yl)piperidine-1-carboxamido)phenyl)(methyl)carbamate (INT 7A)

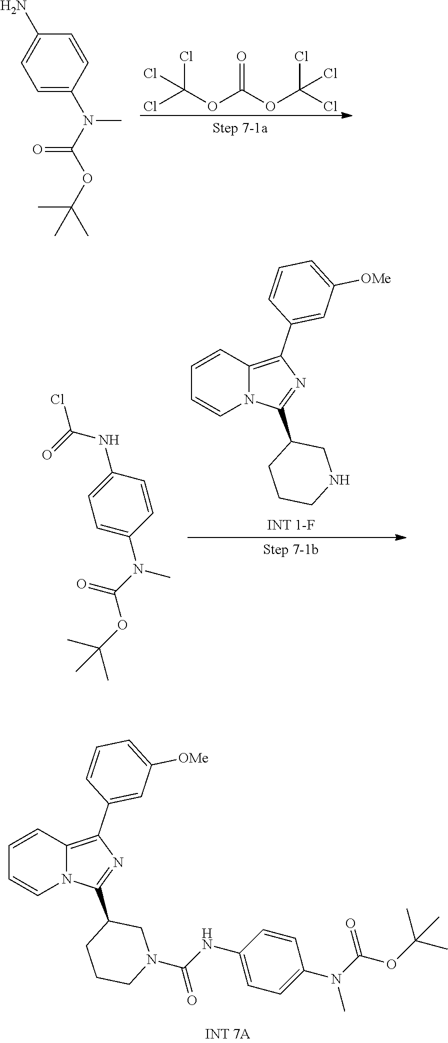

To a stirring solution of tert-butyl (4-aminophenyl)(methyl)carbamate (81 mg, 0.36 mmol) in dioxane (2.0 mL) were added DIEA (235 mg, 1.82 mmol) and bis(trichloromethyl) carbonate (35 mg, 0.12 mmol). After stirring for 5 min, INT 1F (50.0 mg, 0.15 mmol) was added. After stirring for 2 h at room temperature, the mixture was directly loaded onto a SiO₂ column and purified using SiO₂ chromatography (10% MeOH in EA/DCM). The resulting material was further purified by C-18 reverse-phase chromatography (MeOH/H₂O) to provide 17 mg (25.5% yield) of tert-butyl (R)-(4-(3-(1-(3-methoxyphenyl)imidazo[1,5-a]pyridin-3-yl) piperidine-1-carboxamido) phenyl) (methyl) carbamate (INT 7A). LCMS-ESI (m/z) calculated for C₃₂H₃₇N₅O₄: 555.7; found 555.6 [M+H]⁺, t_{R}=6.29 min (Method 3).

### Step 7-2. Synthesis of (R)-3-(1-(3-methoxyphenyl)imidazo[1,5-a]pyridin-3-yl)-N-(4-(methylamino)phenyl)piperidine-1-carboxamide (Compound 7-1)

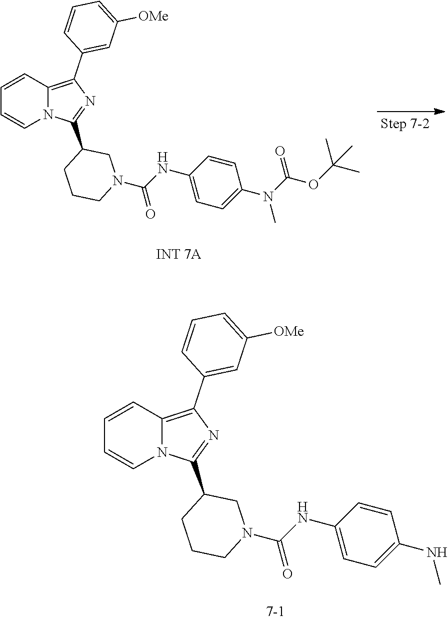 [SMILES: COc1cccc(-c2nc([C@@H]3CCCN(C(=O)Nc4ccc(N(C)C(=O)OC(C)(C)C)cc4)C3)n3ccccc23)c1 ; CNc1ccc(NC(=O)N2CCC[C@@H](c3nc(-c4cccc(OC)c4)c4ccccn34)C2)cc1]

A solution of INT 7A (17 mg, 0.03 mmol) in DCM (1 mL) and TFA (35 mg, 0.3 mmol) was stirred at room temperature for 30 min. The mixture was diluted with sat NaHCO₃ (aq) and extracted into DCM (3×). The combined organic layers were dried (Na₂SO₄), filtered, and concentrated to provide 42 mg (65%) of (R)-3-(1-(3-methoxyphenyl) imidazo[1,5-a]pyridin-3-yl)-N-(4-(methylamino) phenyl) piperidine-1-carboxamide (Compound 7-1). LCMS-ESI (m/z) calculated for C₂₇H₂₉N₅O₂: 455.5; found 456.0 [M+H]⁺, t_{R}=9.76 min (Method 2). ¹H NMR (500 Hz, DMSO-D6): 8.34 (d, J=7.2 Hz, 1H), 8.19 (s, 1H), 7.89 (dd, J=1.2, 9.4 Hz, 1H), 7.45 (dt, J=1.2, 7.8 Hz, 1H), 7.39 (dd, J=1.5, 2.7 Hz, 1H), 7.34 (t, J=7.9 Hz, 1H), 7.18-7.09 (m, 2H), 6.88 (ddd, J=1.0, 6.4, 9.3 Hz, 1H), 6.82 (ddd, J=1.0, 2.6, 8.2 Hz, 1H), 6.73 (ddd, J=1.2, 6.3, 7.3 Hz, 1H), 6.48-6.39 (m, 2H), 5.27 (q, J=5.2 Hz, 1H), 4.34 (d, J=13.1 Hz, 1H), 4.19 (d, J=13.1 Hz, 1H), 3.82 (s, 3H), 3.15 (dd, J=11.0, 13.2 Hz, 1H), 2.91 (t, J=12.4 Hz, 1H), 2.63 (d, J=5.2 Hz, 3H), 2.14 (d, J=12.7 Hz, 1H), 1.93-1.75 (m, 2H), 1.65 (d, J=12.6 Hz, 1H).

The compounds listed in the Table 7 were made using the procedures of Scheme 7.

TABLE 7

| | | Purity | | | | |
|---|---|---|---|---|---|---|
| | Cpd | RT | | Observed | | Purity |
| Structure | No. | (min) | MW | m/z | Ion | Method |
| 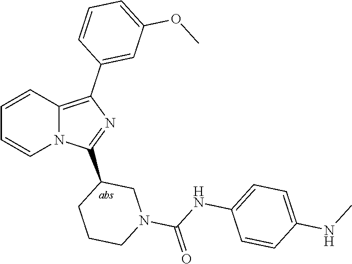 [SMILES: CNc1ccc(NC(=O)N2CCC[C@@H](c3nc(-c4cccc(OC)c4)c4ccccn34)C2)cc1] | 7-1 | 9.76 | 455.2 | 456 | [M + H]⁺ | 2 |

### Example 8

### Synthesis of Compound 8-1 and Other Representative Compounds

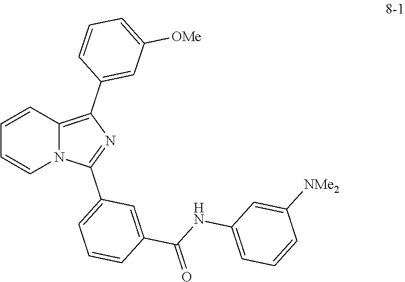 [SMILES: COc1cccc(-c2nc(-c3cccc(C(=O)Nc4cccc(N(C)C)c4)c3)n3ccccc23)c1]

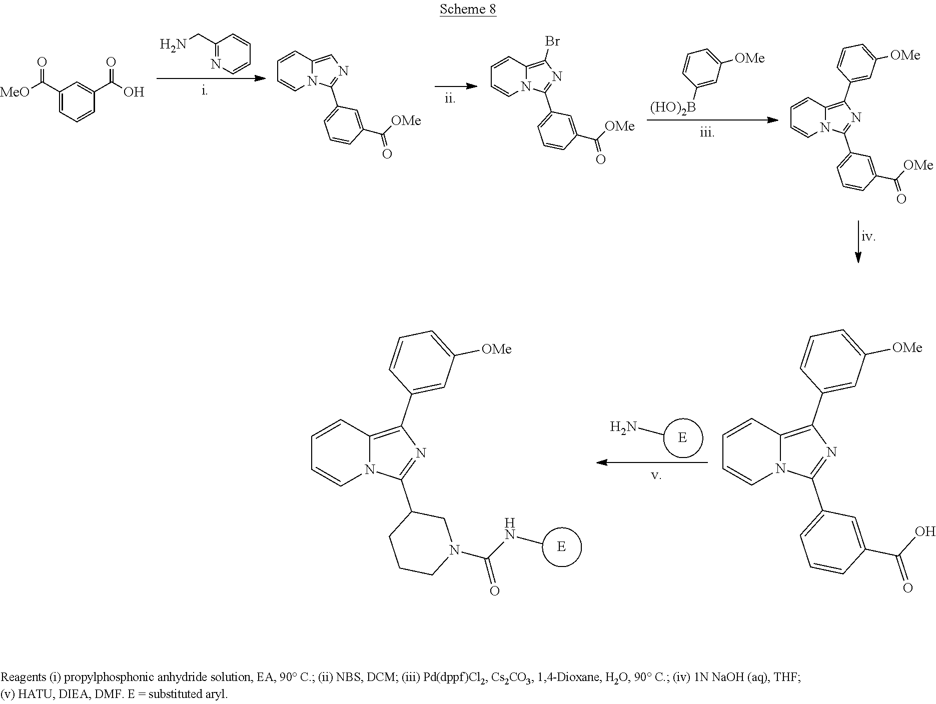

### Step 8-1. Synthesis of methyl 3-(imidazo[1,5-a]pyridin-3-yl)benzoate (INT 8A)

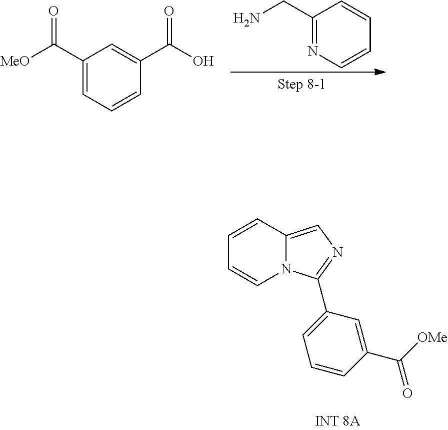 [SMILES: COC(=O)c1cccc(C(=O)O)c1 ; NCc1ccccn1 ; COC(=O)c1cccc(-c2ncc3ccccn23)c1]

Into a 20 mL pressure vial containing a stirring solution of 2-(aminomethyl) pyridine (190 μL, 1.85 mmol) in EA (10 mL) at room temperature was added 3-(methoxycarbonyl)benzoic acid (383 mg, 2.13 mmol). To the resulting suspension was added a solution of propylphosphonic anhydride in EA (1.65 mL, 50% wt, 2.77 mmol). The resulting solution was stirred at room temperature for 1 hour before being heated at reflux (90° C.) for 18 hours. The reaction was heated at 120° C. for 24 hours, then diluted with EA and washed with saturated NaHCO₃ (2×). The organic phase was dried (Na₂SO₄) and concentrated under reduced pressure. The residue was dissolved in DCM and purified by SiO₂ chromatography (MeOH/DCM) to provide 223 mg (48%) of methyl 3-(imidazo[1,5-a]pyridin-3-yl)benzoate (INT 8A) as an amber oil. LCMS-ESI (m/z) calculated for C₁₅H₁₂N₂O₂: 252.0; found 253.2[M+H]⁺, t_{R}=0.595 min (Method 11). ¹H NMR (400 MHZ, DMSO) δ=8.51 (dt, J=1.1, 7.2 Hz, 1H), 8.39 (td, J=0.5, 1.8 Hz, 1H), 8.15 (ddd, J=1.2, 1.9, 7.8 Hz, 1H), 8.03 (ddd, J=1.1, 1.8, 7.8 Hz, 1H), 7.76-7.65 (m, 2H), 7.60 (d, J=0.9 Hz, 1H), 6.89 (ddd, J=0.9, 6.4, 9.1 Hz, 1H), 6.78 (ddd, J=1.3, 6.4, 7.6 Hz, 1H), 3.91 (s, 3H),

### Step 8-2. Synthesis of methyl 3-(1-bromoimidazo[1,5-a]pyridin-3-yl)benzoate (INT 8B)

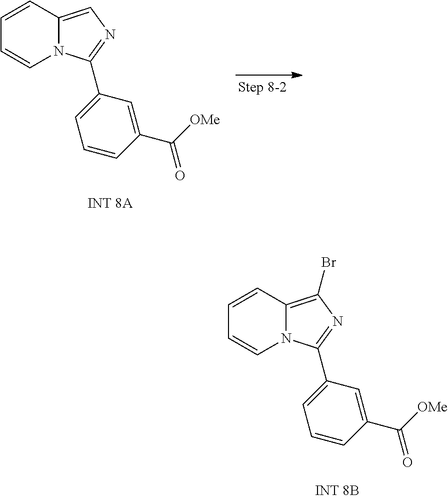 [SMILES: COC(=O)c1cccc(-c2ncc3ccccn23)c1 ; COC(=O)c1cccc(-c2nc(Br)c3ccccn23)c1]

Into a vial containing a stirring solution of INT 8A (217 mg, 860 μmol) in DCM (5 mL) was added N-bromosuccinimide (153 mg, 860 μmol). After stirring for 17 hours, the reaction mixture was purified by SiO₂ chromatography (EA/hexanes) to provide 200 mg (70%) of methyl 3-(1-bromoimidazo[1,5-a]pyridin-3-yl)benzoate (INT 8B) as an off-white solid. LCMS-ESI (m/z) calculated for C₁₅H₁₁BrN₂O₂: 330.0; found 331.0 [M+H]⁺, t_{R}=1.5 min (Method 11). ¹H NMR (400 MHZ, DMSO-D6) δ=8.54 (dd, J=1.1, 7.3 Hz, 1H), 8.36 (d, J=1.8 Hz, 1H), 8.17-8.10 (m, 1H), 8.05 (dt, J=1.4, 7.8 Hz, 1H), 7.73 (t, 1H), 7.52 (dd, J=1.2, 9.2 Hz, 1H), 7.07-6.98 (m, 1H), 6.88 (td, J=1.3, 6.5, 7.0 Hz, 1H), 3.91 (s, 3H).

### Step 8-3. Synthesis of methyl 3-(1-(3-methoxyphenyl)imidazo[1,5-a]pyridin-3-yl)benzoate (INT 8C)

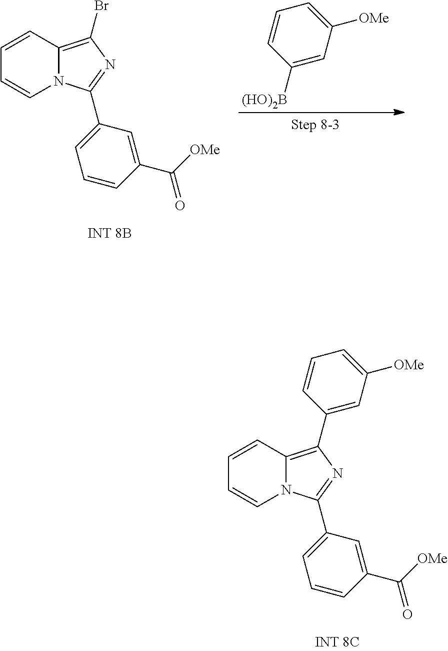 [SMILES: COC(=O)c1cccc(-c2nc(Br)c3ccccn23)c1 ; COc1cccc(B(O)O)c1 ; COC(=O)c1cccc(-c2nc(-c3cccc(OC)c3)c3ccccn23)c1]

A 20 mL pressure vial containing a mixture of INT 8B (195 mg, 589 μmol), 3-methoxyphenylboronic acid (98.4 mg, 648 μmol), [1,1′-Bis(diphenylphosphino)ferrocene]dichloropalladium(II) complex with dichloromethane (96.2 mg, 118 μmol), and cesium carbonate (384 mg, 1.18 mmol) in 1,4-Dioxane (7 mL) and H₂O (0.7 mL) was degassed with nitrogen gas for 5 minutes. The vial was capped, and the mixture heated at 90° C. After 17 hours the reaction mixture was partitioned between EA and H₂O. The organic phase was collected and the aqueous layer was extracted EA (2×). The organic layers were combined, washed with brine, and concentrated under reduced pressure. The residue was dissolved in DCM (9 mL) and purified by SiO₂ chromatography (EA/hexanes) to yield 115 mg (55%) of methyl 3-(1-(3-methoxyphenyl)imidazo[1,5-a]pyridin-3-yl)benzoate (INT 8C) as a sticky yellow foam. LCMS-ESI (m/z) calculated for C₂₂H₁₈N₂O₃: 358.1; found 359.2 [M+H]⁺, t_{R}=5.54 min (Method 12). ¹H NMR (400 MHZ, DMSO-D6) 8=8.52 (dd, J=1.2, 7.3 Hz, 1H), 8.42 (d, J=1.8 Hz, 1H), 8.19 (dt, J=1.6, 7.8 Hz, 1H), 8.11-7.99 (m, 2H), 7.75 (t, J=7.8 Hz, 1H), 7.56-7.50 (m, 1H), 7.47 (dd, J=1.4, 2.7 Hz, 1H), 7.40 (t, J=7.9 Hz, 1H), 7.02 (dd, J=6.4, 9.3 Hz, 1H), 6.93-6.81 (m, 2H), 3.92 (s, 3H), 3.85 (s, 3H).

### Step 8-4. Synthesis of 3-(1-(3-methoxyphenyl)imidazo[1,5-a]pyridin-3-yl)benzoic acid (INT 8D)

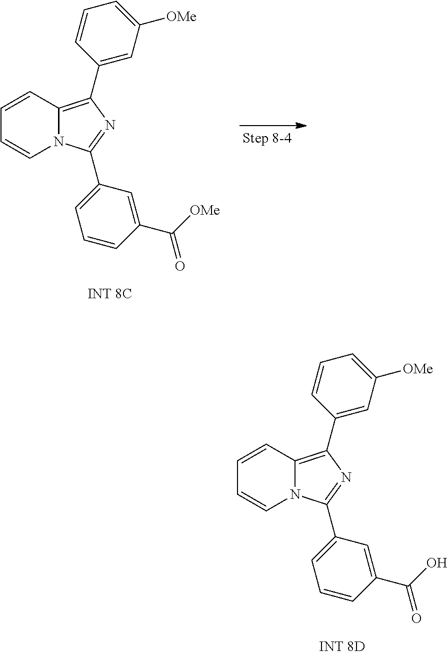 [SMILES: COC(=O)c1cccc(-c2nc(-c3cccc(OC)c3)c3ccccn23)c1 ; COc1cccc(-c2nc(-c3cccc(C(=O)O)c3)n3ccccc23)c1]

A 20 mL vial containing a stirring solution of INT 8C (110 mg, 307 μmol) in THF (6 mL) was charged with 1N sodium hydroxide (0.12 g, 614 μL, 5.00 molar, 3.07 mmol). The resulting yellow solution was stirred at 50° C. for 3 days then was concentrated under reduced pressure. The residue was dissolved in H₂O, and the aqueous solution was acidified to pH 1 with 2N HCl and extracted EA (3×). The organic layers were combined, dried over Na₂SO₄ and concentrated under reduced pressure to yield 98.4 mg (93% yield) of 3-(1-(3-methoxyphenyl)imidazo[1,5-a]pyridin-3-yl)benzoic acid (INT 8D) as a yellow film. LCMS-ESI (m/z) calculated for C₂₁H₁₆N₂O₃: 344.1; found 345.2 [M+H]⁺, t_{R}=0.897 min (Method 11).

### Step 8-5. Synthesis of N-(3-(dimethylamino)phenyl)-3-(1-(3-methoxyphenyl) imidazo[1,5-a]pyridin-3-yl)benzamide (Compound 8-1)

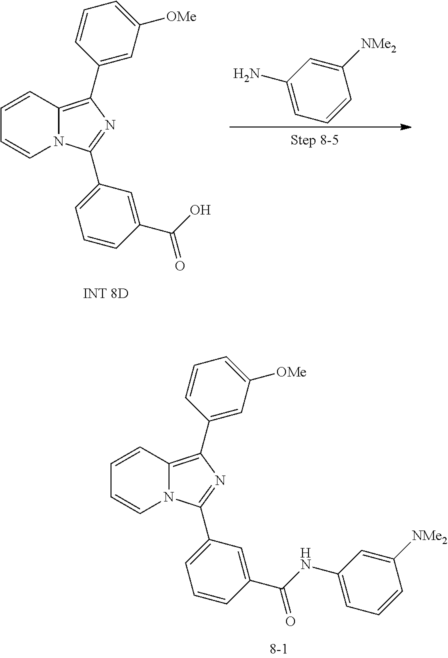 [SMILES: COc1cccc(-c2nc(-c3cccc(C(=O)O)c3)n3ccccc23)c1 ; CN(C)c1cccc(N)c1 ; COc1cccc(-c2nc(-c3cccc(C(=O)Nc4cccc(N(C)C)c4)c3)n3ccccc23)c1]

A vial containing a stirring solution of INT 8D (49 mg, 0.14 mmol) and DIEA (99 μL, 0.57 mmol) in DMF (2 mL) was charged with HATU (54 mg, 0.14 mmol). After stirring for 5 min, the reaction mixture was charged with N,N-dimethyl-m-phenylenediamine dihydrochloride (30 mg, 0.14 mmol). The resulting mixture was stirred at room temperature for 1 hour, passed through a syringe filter into a test tube, and the filtrate was directly purified by prep HPLC. The relevant fractions were directly lyophilized for 2 days to yield 20.7 mg, (31%) of N-(3-(dimethylamino)phenyl)-3-(1-(3-methoxyphenyl)imidazo[1,5-a]pyridin-3-yl)benzamide (Compound 8-1) as a yellow solid. LCMS-ESI (m/z) calculated for C₂₉H₂₆N₄O₂: 462.2; found 462.9 [M+H]⁺, t_{R}=9.097 min (Method 1). ¹H NMR (400 MHZ, DMSO-D6) δ=10.12 (s, 1H), 8.51 (d, J=7.2 Hz, 1H), 8.33 (d, J=1.8 Hz, 1H), 8.05-7.91 (m, 3H), 7.67 (t, J=7.7 Hz, 1H), 7.47 (dt, J=1.3, 7.7 Hz, 1H), 7.41 (t, J=2.1 Hz, 1H), 7.33 (t, J=7.9 Hz, 1H), 7.16 (d, J=2.3 Hz, 1H), 7.14-7.04 (m, 2H), 6.95 (dd, J=6.3, 9.3 Hz, 1H), 6.82 (dd, J=2.6, 8.2 Hz, 1H), 6.81-6.73 (m, 1H), 6.44 (dt, J=2.2, 7.3 Hz, 1H), 3.78 (s, 3H), 2.83 (s, 6H).

The compounds listed in Table 8 were made using the procedures of Scheme 8.

TABLE 8

| | | Purity | | | | |
|---|---|---|---|---|---|---|
| | Cpd | RT | | Observed | | Purity |
| Structure | No. | (min) | MW | m/z | Ion | Method |
| 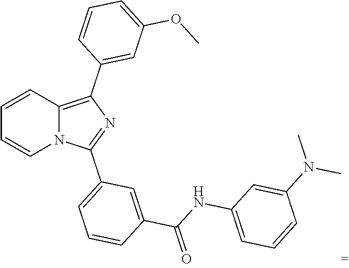 [SMILES: COc1cccc(-c2nc(-c3cccc(C(=O)Nc4cccc(N(C)C)c4)c3)n3ccccc23)c1] | 8-1 | 9.097 | 462.2 | 462.9 | [M + H]⁺ | 1 |
| 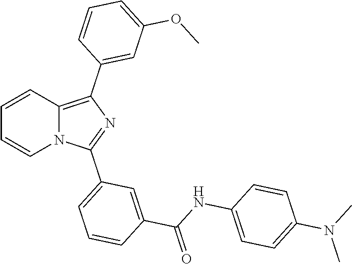 [SMILES: COc1cccc(-c2nc(-c3cccc(C(=O)Nc4ccc(N(C)C)cc4)c3)n3ccccc23)c1] | 8-2 | 7.427 | 462.2 | 460.8 | [M − H]⁻ | 10 |

### Example 9

### Synthesis of Compound 9-1 and Other Representative Compounds

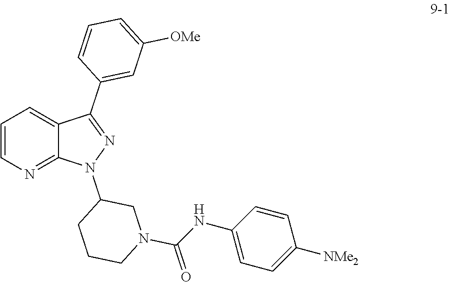 [SMILES: COc1cccc(-c2nn(C3CCCN(C(=O)Nc4ccc(N(C)C)cc4)C3)c3ncccc23)c1]

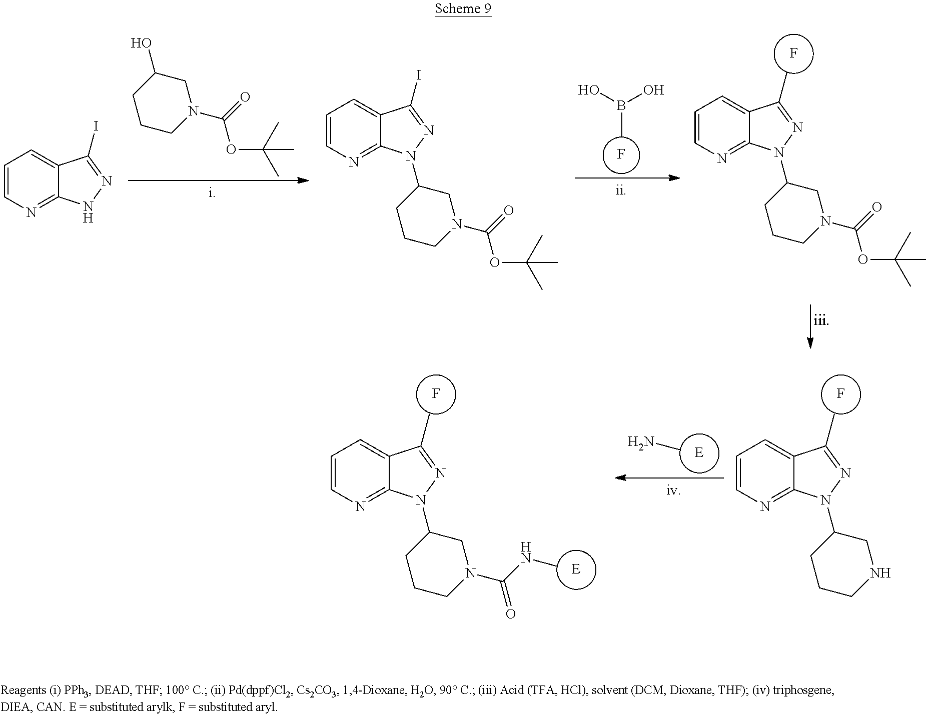

### Step 9-1. Synthesis of tert-butyl 3-(3-iodo-1H-pyrazolo[3,4-b]pyridin-1-yl)piperidine-1-carboxylate (INT 9A)

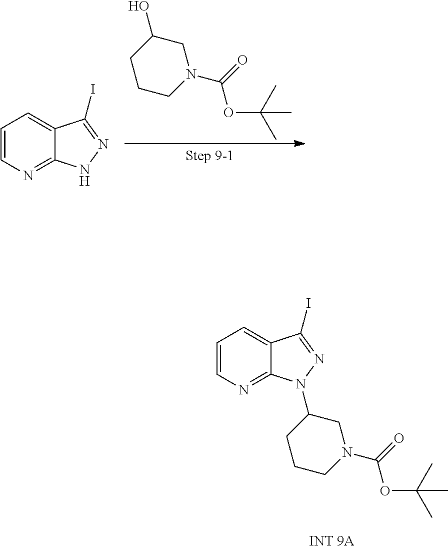 [SMILES: Ic1n[nH]c2ncccc12 ; CC(C)(C)OC(=O)N1CCCC(O)C1 ; CC(C)(C)OC(=O)N1CCCC(n2nc(I)c3cccnc32)C1]

A 20 mL pressure vial containing a stirring solution of 3-iodo-1H-pyrazolo[3,4-b]pyridine (300 mg, 1.22 mmol) and 1-Boc-3-hydroxypiperidine (246 mg, 1.22 mmol) in THF (5 mL) was charged with triphenylphosphine (482 mg, 1.84 mmol) and diisopropyl azodicarboxylate (357 μL, 1.84 mmol). After stirring for 18 hours, the reaction mixture was poured into H₂O and extracted with EA (3×). The organic layers were combined, washed with brine, and concentrated under reduced pressure. The residue was dissolved in DCM and purified by SiO₂ chromatography (EA/hexanes) to yield 235 mg (45%) of tert-butyl 3-(3-iodo-1H-pyrazolo[3,4-b]pyridin-1-yl)piperidine-1-carboxylate (INT 9A) as a white foam. LCMS-ESI (m/z) calculated for C₁₆H₂₁IN₄O₂: 428.1; found 429.2 [M+H]⁺, t_{R}=1.16 min (Method 11). ¹H NMR (400 MHZ, DMSO-D6) δ 8.63 (dd, J=1.5, 4.4 Hz, 1H), 7.96 (dd, J=1.6, 8.1 Hz, 1H), 7.33 (dd, J=4.5, 8.1 Hz, 1H), 4.84 (dq, J=4.2, 4.7, 9.6 Hz, 1H), 3.92 (s, 1H), 3.78 (s, 3H), 3.32 (s, 1H), 2.21 (td, J=3.8, 10.8 Hz, 1H), 2.11 (dd, J=4.4, 13.2 Hz, 1H), 1.93 (s, 1H), 1.66-1.50 (m, 1H), 1.41 (s, 2H), 1.28 (s, 5H), 1.26-1.14 (m, 1H).

### Step 9-2. Synthesis of tert-butyl 3-(3-(3-methoxyphenyl)-1H-pyrazolo[3,4-b]pyridin-1-yl)piperidine-1-carboxylate (INT 9B)

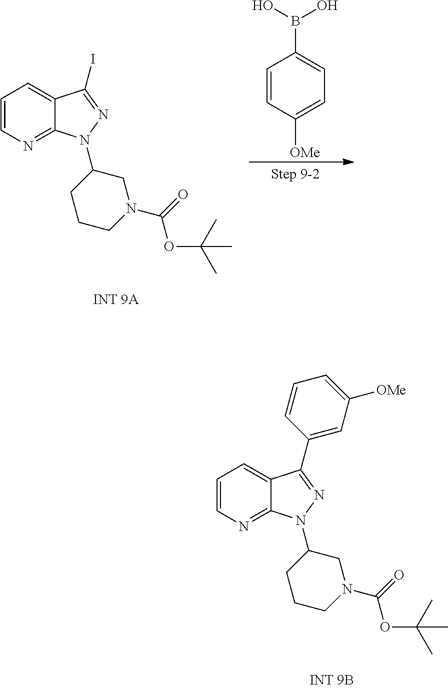 [SMILES: CC(C)(C)OC(=O)N1CCCC(n2nc(I)c3cccnc32)C1 ; COc1ccc(B(O)O)cc1 ; COc1cccc(-c2nn(C3CCCN(C(=O)OC(C)(C)C)C3)c3ncccc23)c1]

A 20 mL pressure vial containing a mixture of INT 9A (229 mg, 535 μmol), 3-methoxyphenylboronic acid (89.4 mg, 588 μmol), [1,1′-Bis (diphenylphosphino) ferrocene] dichloropalladium(II) complex with dichloromethane (87.3 mg, 107 μmol), and cesium carbonate (348 mg, 1.07 mmol) in 1,4-Dioxane (5 mL) and H₂O (0.5 mL) was degassed with N₂ gas for 5 min. The vial was capped, and the mixture was heated at 90° C. After stirring for 17 hours, the reaction mixture was partitioned between EA and H₂O. The organic phase was collected, and the aqueous layer was extracted with EA (2×). The organic layers were combined, washed with brine, and concentrated under reduced pressure. The residue was dissolved in DCM and purified by SiO₂ chromatography (EA/hexanes) to yield 151 mg (69%) of tert-butyl 3-(3-(3-methoxyphenyl)-1H-pyrazolo[3,4-b]pyridin-1-yl)piperidine-1-carboxylate (INT 9B) as a white foam. LCMS-ESI (m/z) calculated for C₂₃H₂₈N₄O₃: 408.2; found 409.2 [M+H]⁺, t_{R}=1.22 min (Method 11).

### Step 9-3. Synthesis of 3-(3-methoxyphenyl)-1-(piperidin-3-yl)-1H-pyrazolo[3,4-b]pyridine (INT 9C)

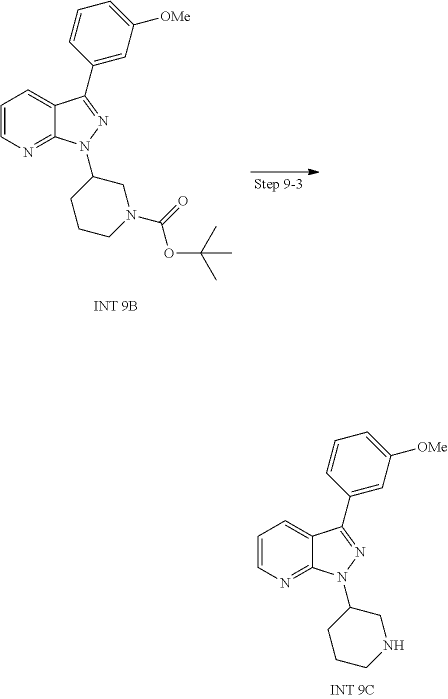 [SMILES: COc1cccc(-c2nn(C3CCCN(C(=O)OC(C)(C)C)C3)c3ncccc23)c1 ; COc1cccc(-c2nn(C3CCCNC3)c3ncccc23)c1]

To a 20 mL vial containing a stirring solution of INT 9B (146 mg, 357 μmol) in 1,4-Dioxane (4 mL) was added hydrogen chloride in 1,4-dioxane (894 μL, 4.0 molar, 3.6 mmol). The resulting mixture was allowed to stir at 50° C. for 18 hours. The reaction mixture was charged with diethyl ether and filtered. The filter cake was washed with diethyl ether and dried to yield 93.6 mg (76%) of 3-(3-methoxyphenyl)-1-(piperidin-3-yl)-1H-pyrazolo[3,4-b]pyridine hydrochloride (INT 9C) as a yellow solid. The product was taken forward to the next step without further purification. LCMS-ESI (m/z) calculated for C₁₈H₂₀N₄O: 308.2; found 309.2 [M+H]⁺, t_{R}=0.585 min (Method 11).

### Step 9-4. Synthesis of N-(4-(dimethylamino)phenyl)-3-(3-(3-methoxyphenyl)-1H-pyrazolo[3,4-b]pyridin-1-yl)piperidine-1-carboxamide (Compound 9-1)

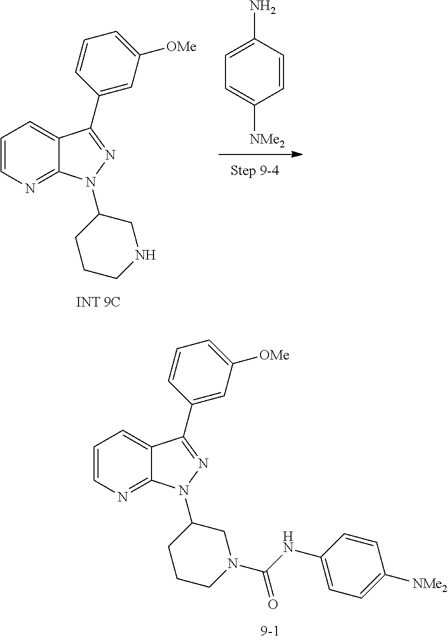 [SMILES: COc1cccc(-c2nn(C3CCCNC3)c3ncccc23)c1 ; CN(C)c1ccc(N)cc1 ; COc1cccc(-c2nn(C3CCCN(C(=O)Nc4ccc(N(C)C)cc4)C3)c3ncccc23)c1]

A vial containing a mixture of N,N-dimethyl-p-phenylenediamine (18 mg, 0.13 mmol) in MeCN (2 mL) was charged with DIEA (0.11 mL, 0.65 mmol). The mixture was cooled to 0° C., and triphosgene (39 mg, 0.13 mmol) was charged in one portion. The reaction mixture was stirred at 0° C. for 10 minutes, then was charged with INT 9C (45 mg, 0.13 mmol), followed by DIEA (0.04 mL, 26 mmol). The reaction mixture was stirred at 50° C. for 1.5 hours, passed through a syringe filter into a test tube, and the filtrate was directly purified by prep HPLC. The relevant fractions were directly lyophilized for 2 days to yield 36.4 mg (59%) of N-(4-(dimethylamino)phenyl)-3-(3-(3-methoxyphenyl)-1H-pyrazolo[3,4-b]pyridin-1-yl)piperidine-1-carboxamide (Compound 9-1) as a white solid. LCMS-ESI (m/z) calculated for C₂₇H₃₀N₆O₂: 470.2; found 471.0 [M+H]⁺, t_{R}=6.18 min (Method 1). ¹H NMR (400 MHZ, DMSO-D6) δ 8.66-8.57 (m, 2H), 8.30 (s, 1H), 7.61 (dt, J=1.2, 7.8 Hz, 1H), 7.51 (dd, J=1.5, 2.6 Hz, 1H), 7.47 (t, J=7.9 Hz, 1H), 7.35 (dd, J=4.5, 8.2 Hz, 1H), 7.27-7.19 (m, 2H), 7.03 (ddd, J=1.0, 2.6, 8.3 Hz, 1H), 6.69-6.60 (m, 2H), 5.03-4.91 (m, 1H), 4.39-4.32 (m, 1H), 4.17 (d, J=13.3 Hz, 1H), 3.87 (s, 3H), 3.42 (dd, J=10.6, 12.8 Hz, 1H), 2.98 (t, J=12.0 Hz, 1H), 2.82 (s, 6H), 2.29 (td, J=4.0, 12.2 Hz, 1H), 1.93 (d, J=12.9 Hz, 1H), 1.68 (s, 1H).

The compounds listed in Table 9 wase made using the procedures of Scheme 9:

TABLE 9

| | | Purity | | | | |
|---|---|---|---|---|---|---|
| | Cpd | RT | | Observed | | Purity |
| Structure | No. | (min) | MW | m/z | Ion | Method |
| 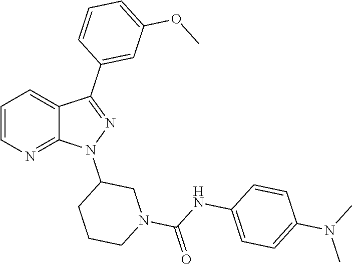 [SMILES: COc1cccc(-c2nn(C3CCCN(C(=O)Nc4ccc(N(C)C)cc4)C3)c3ncccc23)c1] | 9-1 | 6.182 | 470.2 | 471 | [M + H]⁺ | 1 |
| 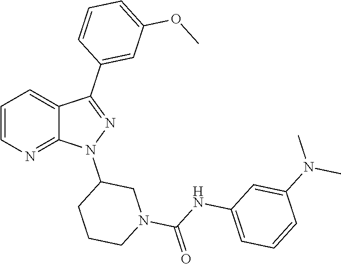 [SMILES: COc1cccc(-c2nn(C3CCCN(C(=O)Nc4cccc(N(C)C)c4)C3)c3ncccc23)c1] | 9-2 | 7.242 | 470.2 | 471 | [M + H]⁺ | 1 |

### Example 10

### Synthesis of Compound 10-1 and Other Representative Compounds

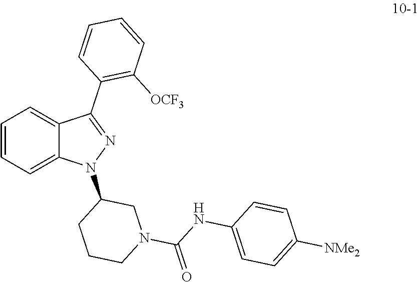 [SMILES: CN(C)c1ccc(NC(=O)N2CCC[C@@H](n3nc(-c4ccccc4OC(F)(F)F)c4ccccc43)C2)cc1]

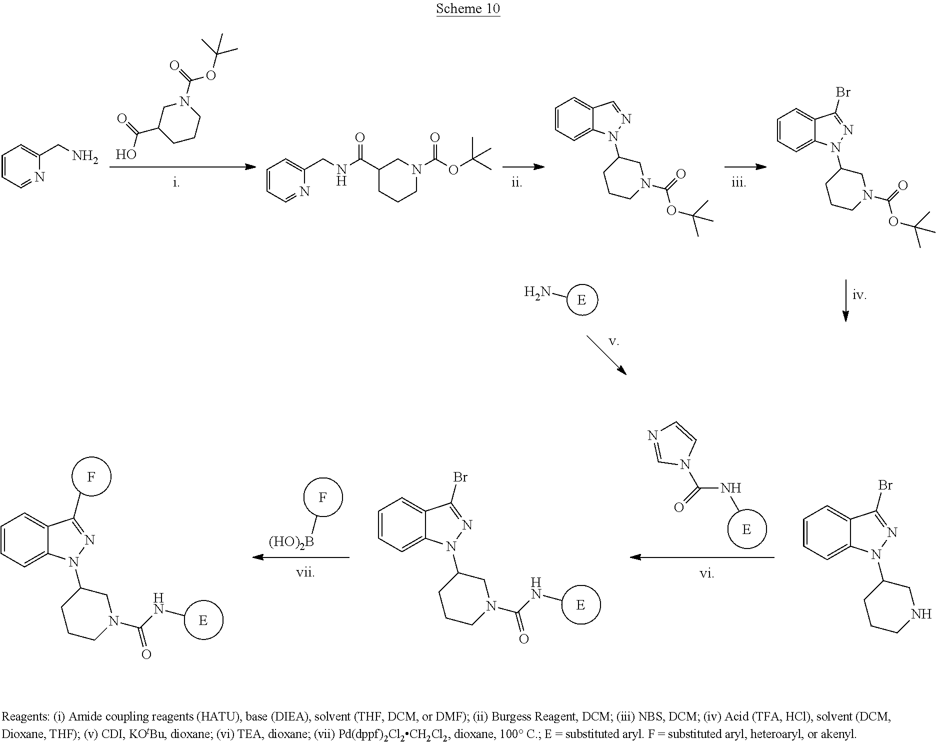

### Step 10-1. Synthesis of tert-butyl (R)-3-((pyridin-2-ylmethyl)carbamoyl)piperidine-1-carboxylate (INT 10A)

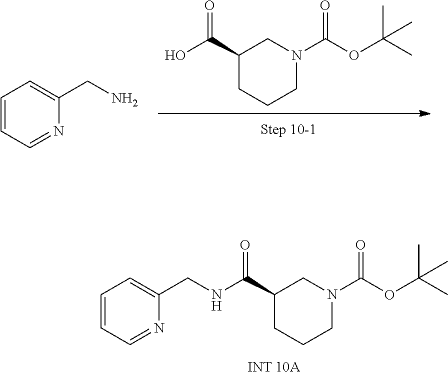 [SMILES: NCc1ccccn1 ; CC(C)(C)OC(=O)N1CCC[C@@H](C(=O)O)C1 ; CC(C)(C)OC(=O)N1CCC[C@@H](C(=O)NCc2ccccn2)C1]

(R)-1-(tert-butoxycarbonyl)piperidine-3-carboxylic acid (3.07 g, 13.4 mmol) and HATU (5.6 g, 13.4 mmol) were added to a flask containing DMF (8 mL). After 5 min, DIEA (2.08 g, 16.1 mmol) and pyridin-2-ylmethanamine (1.45 g, 13.41 mmol) were added. After stirring at room temperature for 20 min, the reaction mixture was diluted with a saturated solution of NaHCO₃ (aq) and extracted with EA (3×). The combined organic extracts were washed with H₂O and brine, then dried (Na₂SO₄), filtered, and concentrated to provide a crude residue that was purified by SiO₂ chromatography (MeOH/DCM) to provide 2.6 g (81%) of tert-butyl (R)-3-((pyridin-2-ylmethyl)carbamoyl) piperidine-1-carboxylate (INT 10A). LCMS-ESI (m/z) calculated for C₁₇H₂₅N₃O₃: 319.4; found 320.4 [M+H]⁺, t_{R}=5.3 min (Method 3).

### Step 10-2. Synthesis of tert-butyl (R)-3-(1H-indazol-1-yl)piperidine-1-carboxylate (INT 10B)

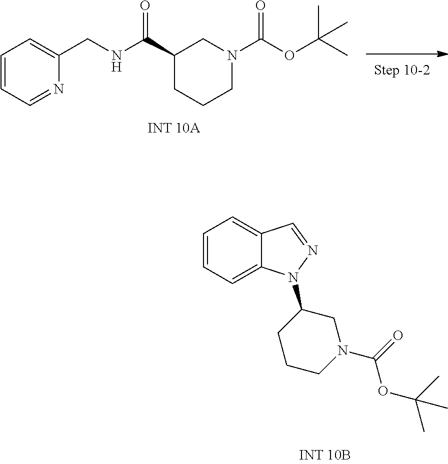 [SMILES: CC(C)(C)OC(=O)N1CCC[C@@H](C(=O)NCc2ccccn2)C1 ; CC(C)(C)OC(=O)N1CCC[C@@H](n2ncc3ccccc32)C1]

To a vial containing INT 10A (2.59 g, 3.1 mmol) in DCM (20 ml) was added Burgess reagent (2.12 g, 8.9 mmol). After stirring for 18 hr at room temperature, additional Burgess reagent was added (469 mg, 1.9 mmol). After 4 hr, the mixture was concentrated and purified by SiO₂ chromatography (EA/hexane) to afford 1.98 g, (51%) of tert-butyl (R)-3-(1H-indazol-1-yl)piperidine-1-carboxylate (INT 10B) as an off-white solid. LCMS-ESI (m/z) calculated for C₁₇H₂₃N₃O₂: 301.4; found 302.5 [M+H]⁺, t_{R}=5.28 min (Method 3).

### Step 10-3. Synthesis of tert-butyl (R)-3-(3-bromo-1H-indazol-1-yl)piperidine-1-carboxylate (INT 10C)

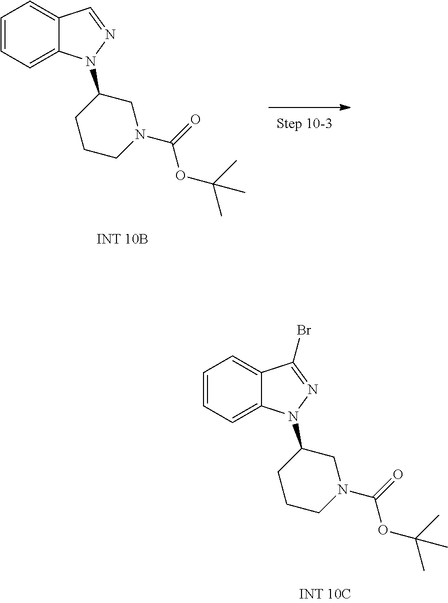 [SMILES: CC(C)(C)OC(=O)N1CCC[C@@H](n2ncc3ccccc32)C1 ; CC(C)(C)OC(=O)N1CCC[C@@H](n2nc(Br)c3ccccc32)C1]

To a stirring solution of INT 10B (1.98 g, 6.57 mmol) in DCM (10 mL) was added NBS (1.29 g, 7.23 mmol). After stirring for 30 min, the reaction mixture was concentrated and purified by SiO₂ chromatography (EA/hexanes) to afford 1.05 g (42%) of tert-butyl (R)-3-(3-bromo-1H-indazol-1-yl)piperidine-1-carboxylate (INT 10C). LCMS-ESI (m/z) calculated for C₁₇H₂₂BrN₃O₂: 380.2; found 382 [M+H]⁺, t_{R}=6.05 min (Method 3).

### Step 10-4. Synthesis of (R)-3-bromo-1-(piperidin-3-yl)-1H-indazole (INT 10D)

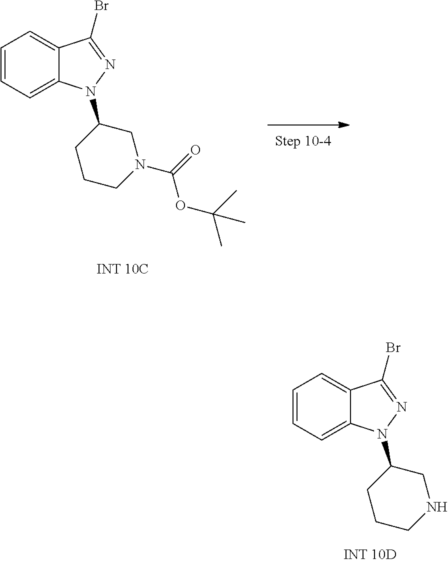 [SMILES: CC(C)(C)OC(=O)N1CCC[C@@H](n2nc(Br)c3ccccc32)C1 ; Brc1nn([C@@H]2CCCNC2)c2ccccc12]

INT 10C (1.11 g, 2.92 mmol) was dissolved in TFA (5 mL). After stirring for 1 h at room temperature, the reaction mixture was concentrated to afford 1.15 g (99%) of (R)-3-bromo-1-(piperidin-3-yl)-1H-indazole (INT 10D) that was used in the next step without further purification. LCMS-ESI (m/z) calculated for C12H₁₄BrN₃: 280.2; found 281.9 [M+H]⁺, t_{R}=5.13 min (Method 3).

### Step 10-5. Synthesis of (R)-3-(3-bromo-1H-indazol-1-yl)-N-(4-(dimethylamino)phenyl)piperidine-1-carboxamide (INT 10E)

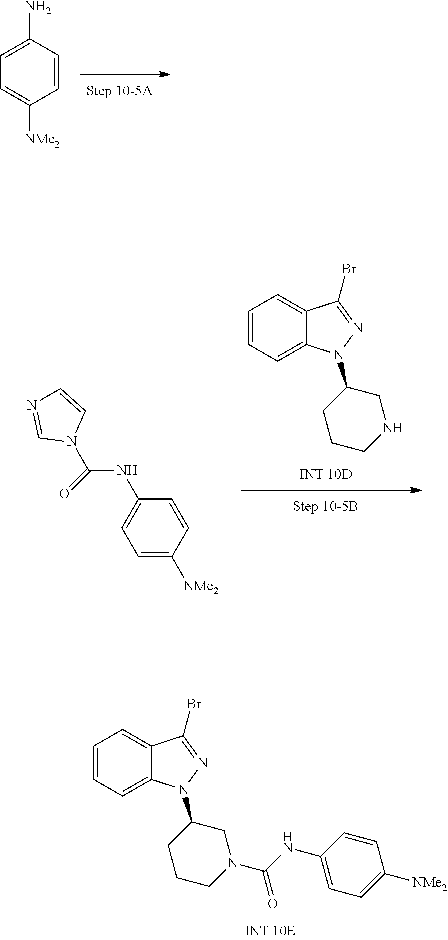 [SMILES: CN(C)c1ccc(N)cc1 ; CN(C)c1ccc(NC(=O)n2ccnc2)cc1 ; Brc1nn([C@@H]2CCCNC2)c2ccccc12 ; CN(C)c1ccc(NC(=O)N2CCC[C@@H](n3nc(Br)c4ccccc43)C2)cc1]

To a stirring solution of N1,N1-dimethylbenzene-1,4-diamine (2.5 g, 18.4 mmol) in dioxane (10 mL) was added KO′Bu (2.27 g, 20.2 mmol). After stirring for 5 min, CDI (2.98 g, 18.4 mmol) was added. After stirring for 2 h, Et₃N (5.6 g, 55 mmol) and INT 10D (2.62 g, 9.3 mmol) were added. After stirring overnight, the reaction mixture was concentrated, suspended in DCM and washed with H₂O (3×). The organic layer was dried (Na₂SO₄), filtered, concentrated, and purified by SiO₂ chromatography (10% MeOH in EA/hexanes) and then C-18 reverse phase chromatography (MeOH/H₂O) to afford 2.7 g (33%) of (R)-3-(3-bromo-1H-indazol-1-yl)-N-(4-(dimethylamino) phenyl)piperidine-1-carboxamide (INT 10E). LCMS-ESI (m/z) calculated for C₂₁H₂₄BrN₅O: 442.4; found 441.9 [M+H]⁺, t_{R}=5.71 min (Method 3).

### Step 10-6. Synthesis of (R)—N-(4-(dimethylamino)phenyl)-3-(3-(2-(trifluoromethoxy)phenyl)-1H-indazol-1-yl)piperidine-1-carboxamide (Compound 10-12)

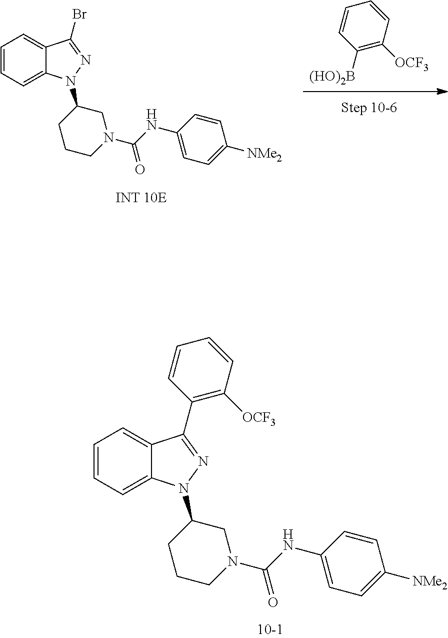 [SMILES: CN(C)c1ccc(NC(=O)N2CCC[C@@H](n3nc(Br)c4ccccc43)C2)cc1 ; OB(O)c1ccccc1OC(F)(F)F ; CN(C)c1ccc(NC(=O)N2CCC[C@@H](n3nc(-c4ccccc4OC(F)(F)F)c4ccccc43)C2)cc1]

INT 10E (44 mg, 0.10 mmol), (2-(trifluoromethoxy)phenyl) boronic acid (25 mg, 0.12 mmol), Pd(dppf)Cl₂ (7 mg, 0.01 mmol) and K₃PO₄ (64 mg, 0.30 mmol) were dissolved in a solution of dioxane (2 mL) and H₂O (1 mL) in a sealable tube. The tube was purged with N₂, sealed, and heated at 110° C. for 20 min. The mixture was cooled, diluted with EA, washed with saturated NaHCO₃ (aq) and brine, then dried (Na₂SO₂), filtered, purified by SiO₂ chromatography (EA/hexanes) then RP-C18 (MeOH/H₂O) to afford 19 mg (36%) of (R)—N-(4-(dimethylamino)phenyl)-3-(3-(2-(trifluoromethoxy)phenyl)-1H-indazol-1-yl)piperidine-1-carboxamide (Compound 10-1). LCMS-ESI (m/z) calculated for C28H₂₈F₃N₅O₂: 523.2; found 523.8 [M+H]⁺, t_{R}=10.79 min (Method 2).

The compounds listed in Table 10 were made using the procedures of Scheme 10:

TABLE 10

| | | Purity | | | | |
|---|---|---|---|---|---|---|
| | Cpd | RT | | Observed | | Purity |
| Structure | No. | (min) | MW | m/z | Ion | Method |
| 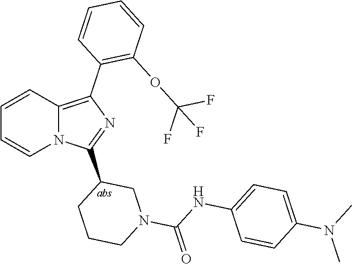 [SMILES: CN(C)c1ccc(NC(=O)N2CCC[C@@H](c3nc(-c4ccccc4OC(F)(F)F)c4ccccn34)C2)cc1] | 10-1 | 11.28 | 523.2 | 523.8 | [M + H]⁺ | 2 |
| 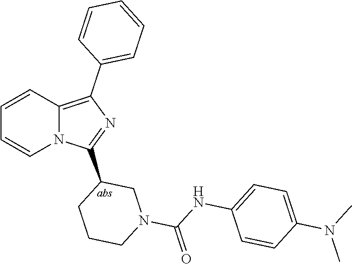 [SMILES: CN(C)c1ccc(NC(=O)N2CCC[C@@H](c3nc(-c4ccccc4)c4ccccn34)C2)cc1] | 10-2 | 9.47 | 439.2 | 440.2 | [M + H]⁺ | 2 |
| 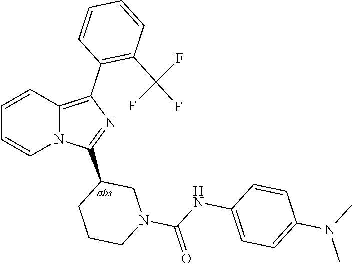 [SMILES: CN(C)c1ccc(NC(=O)N2CCC[C@@H](c3nc(-c4ccccc4C(F)(F)F)c4ccccn34)C2)cc1] | 10-3 | 10.44 | 507.2 | 507.8 | [M + H]⁺ | 2 |
| 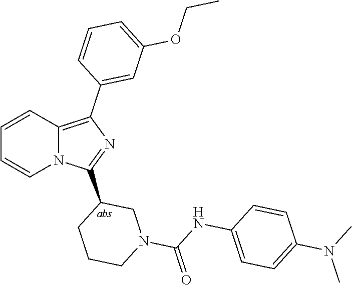 [SMILES: CCOc1cccc(-c2nc([C@@H]3CCCN(C(=O)Nc4ccc(N(C)C)cc4)C3)n3ccccc23)c1] | 10-4 | 9.98 | 483.3 | 484.1 | [M + H]⁺ | 2 |
| 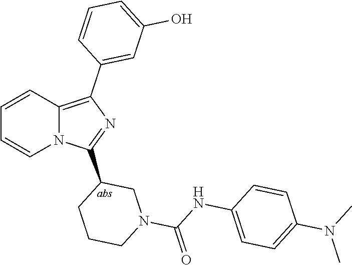 [SMILES: CN(C)c1ccc(NC(=O)N2CCC[C@@H](c3nc(-c4cccc(O)c4)c4ccccn34)C2)cc1] | 10-5 | 8.56 | 455.2 | 456 | [M + H]⁺ | 2 |
| 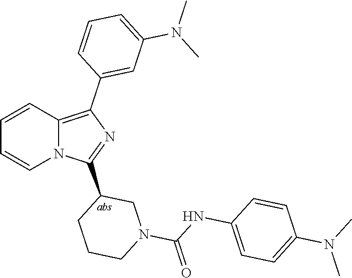 [SMILES: CN(C)c1ccc(NC(=O)N2CCC[C@@H](c3nc(-c4cccc(N(C)C)c4)c4ccccn34)C2)cc1] | 10-6 | 8.64 | 482.3 | 483 | [M + H]⁺ | 2 |
| 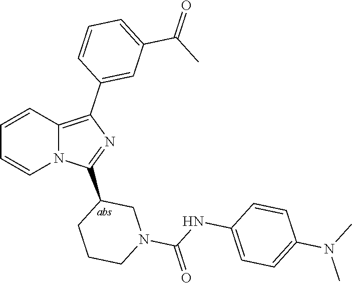 [SMILES: CC(=O)c1cccc(-c2nc([C@@H]3CCCN(C(=O)Nc4ccc(N(C)C)cc4)C3)n3ccccc23)c1] | 10-7 | 9.79 | 481.2 | 481.9 | [M + H]⁺ | 2 |
| 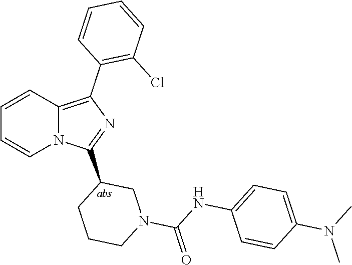 [SMILES: CN(C)c1ccc(NC(=O)N2CCC[C@@H](c3nc(-c4ccccc4Cl)c4ccccn34)C2)cc1] | 10-8 | 10.31 | 473.2 | 475 | [M + H]⁺ | 2 |
| 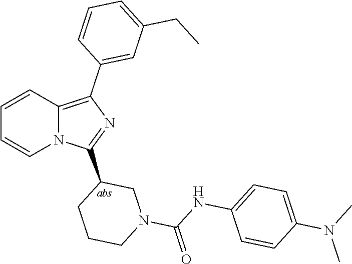 [SMILES: CCc1cccc(-c2nc([C@@H]3CCCN(C(=O)Nc4ccc(N(C)C)cc4)C3)n3ccccc23)c1] | 10-9 | 10.99 | 467.3 | 467.8 | [M + H]⁺ | 2 |
| 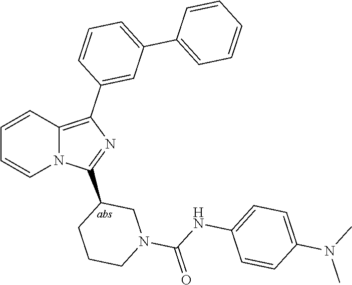 [SMILES: CN(C)c1ccc(NC(=O)N2CCC[C@@H](c3nc(-c4cccc(-c5ccccc5)c4)c4ccccn34)C2)cc1] | 10-10 | 11.8 | 515.3 | 516 | [M + H]⁺ | 2 |
| 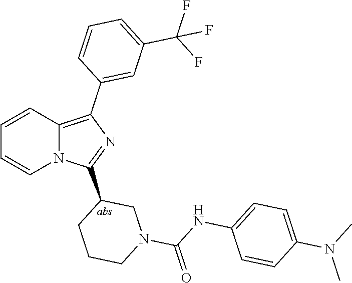 [SMILES: CN(C)c1ccc(NC(=O)N2CCC[C@@H](c3nc(-c4cccc(C(F)(F)F)c4)c4ccccn34)C2)cc1] | 10-11 | 12.09 | 507.2 | 507.5 | [M + H]⁺ | 2 |
| 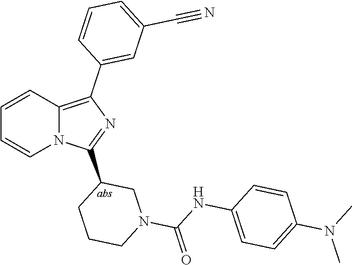 [SMILES: CN(C)c1ccc(NC(=O)N2CCC[C@@H](c3nc(-c4cccc(C#N)c4)c4ccccn34)C2)cc1] | 10-12 | 10.65 | 464.2 | 464.9 | [M + H]⁺ | 2 |
| 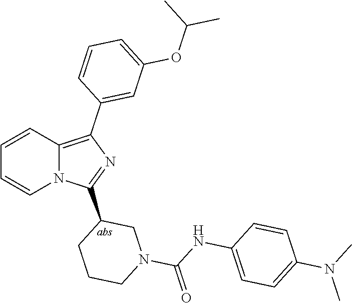 [SMILES: CC(C)Oc1cccc(-c2nc([C@@H]3CCCN(C(=O)Nc4ccc(N(C)C)cc4)C3)n3ccccc23)c1] | 10-13 | 10.9 | 497.3 | 497.9 | [M + H]⁺ | 2 |
| 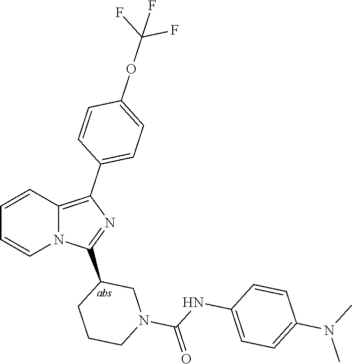 [SMILES: CN(C)c1ccc(NC(=O)N2CCC[C@@H](c3nc(-c4ccc(OC(F)(F)F)cc4)c4ccccn34)C2)cc1] | 10-14 | 11.49 | 523.2 | 523.7 | [M + H]⁺ | 2 |
| 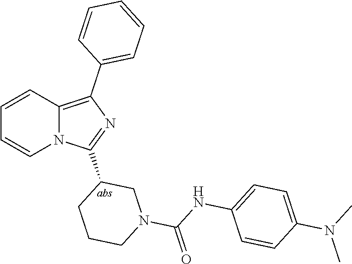 [SMILES: CN(C)c1ccc(NC(=O)N2CCC[C@H](c3nc(-c4ccccc4)c4ccccn34)C2)cc1] | 10-15 | 9.21 | 439.2 | 439.9 | [M + H]⁺ | 2 |
| 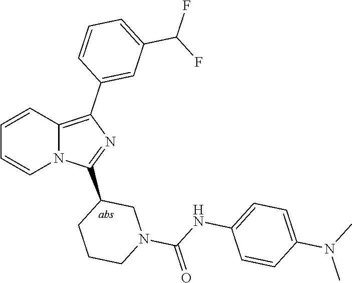 [SMILES: CN(C)c1ccc(NC(=O)N2CCC[C@@H](c3nc(-c4cccc(C(F)F)c4)c4ccccn34)C2)cc1] | 10-16 | 10.79 | 489.2 | 490 | [M + H]⁺ | 2 |
| 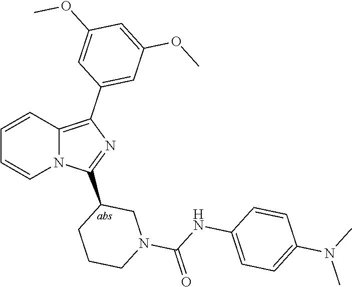 [SMILES: COc1cc(OC)cc(-c2nc([C@@H]3CCCN(C(=O)Nc4ccc(N(C)C)cc4)C3)n3ccccc23)c1] | 10-17 | 10.14 | 499.3 | 499.9 | [M + H]⁺ | 2 |
| 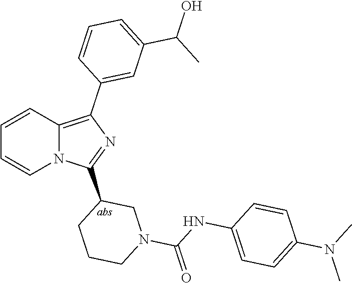 [SMILES: CC(O)c1cccc(-c2nc([C@@H]3CCCN(C(=O)Nc4ccc(N(C)C)cc4)C3)n3ccccc23)c1] | 10-18 | 8.91 | 483.3 | 484 | [M + H]⁺ | 2 |
| 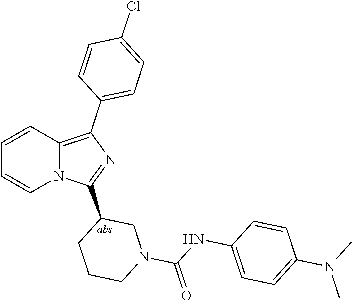 [SMILES: CN(C)c1ccc(NC(=O)N2CCC[C@@H](c3nc(-c4ccc(Cl)cc4)c4ccccn34)C2)cc1] | 10-19 | 10.92 | 473.2 | 475.1 | [M + H]⁺ | 2 |
| 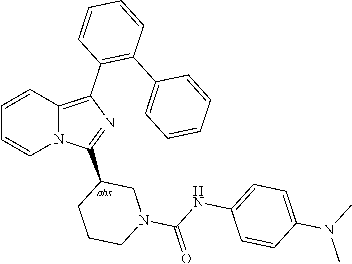 [SMILES: CN(C)c1ccc(NC(=O)N2CCC[C@@H](c3nc(-c4ccccc4-c4ccccc4)c4ccccn34)C2)cc1] | 10-20 | 10.63 | 515.3 | 516 | [M + H]⁺ | 2 |
| 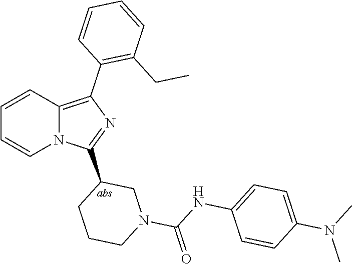 [SMILES: CCc1ccccc1-c1nc([C@@H]2CCCN(C(=O)Nc3ccc(N(C)C)cc3)C2)n2ccccc12] | 10-21 | 10.1 | 467.3 | 467.7 | [M + H]⁺ | 2 |
| 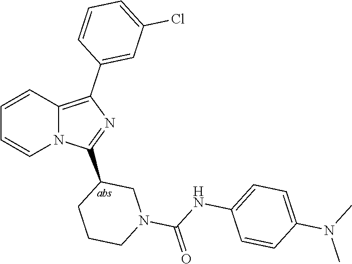 [SMILES: CN(C)c1ccc(NC(=O)N2CCC[C@@H](c3nc(-c4cccc(Cl)c4)c4ccccn34)C2)cc1] | 10-22 | 10.93 | 473.2 | 475.5 | [M + H]⁺ | 2 |
| 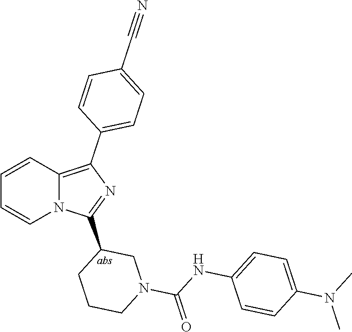 [SMILES: CN(C)c1ccc(NC(=O)N2CCC[C@@H](c3nc(-c4ccc(C#N)cc4)c4ccccn34)C2)cc1] | 10-23 | 10.77 | 464.2 | 465.1 | [M + H]⁺ | 2 |
| 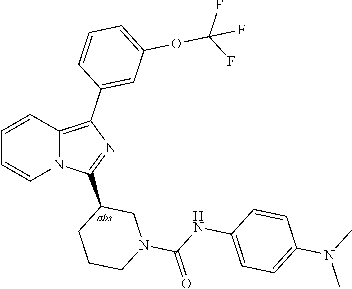 [SMILES: CN(C)c1ccc(NC(=O)N2CCC[C@@H](c3nc(-c4cccc(OC(F)(F)F)c4)c4ccccn34)C2)cc1] | 10-24 | 11.39 | 523.2 | 524 | [M + H]⁺ | 2 |
| 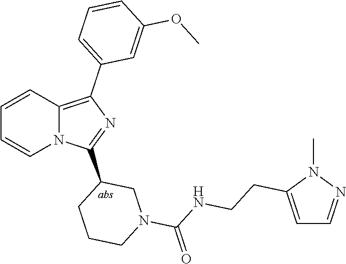 [SMILES: COc1cccc(-c2nc([C@@H]3CCCN(C(=O)NCCc4ccnn4C)C3)n3ccccc23)c1] | 10-25 | 10.54 | 458.2 | 459.1 | [M + H]⁺ | 2 |
| 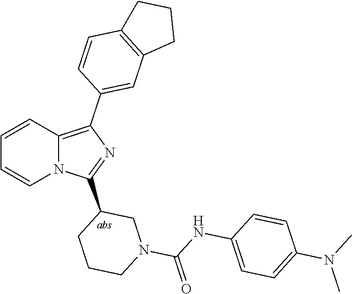 [SMILES: CN(C)c1ccc(NC(=O)N2CCC[C@@H](c3nc(-c4ccc5c(c4)CCC5)c4ccccn34)C2)cc1] | 10-26 | 9.56 | 479.3 | 479.9 | [M + H]⁺ | 2 |
| 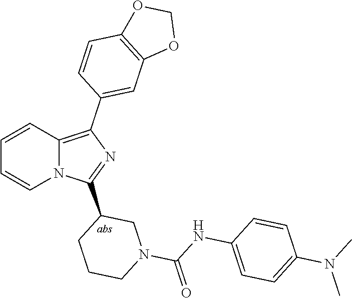 [SMILES: CN(C)c1ccc(NC(=O)N2CCC[C@@H](c3nc(-c4ccc5c(c4)OCO5)c4ccccn34)C2)cc1] | 10-27 | 8.89 | 483.2 | 484 | [M + H]⁺ | 2 |
| 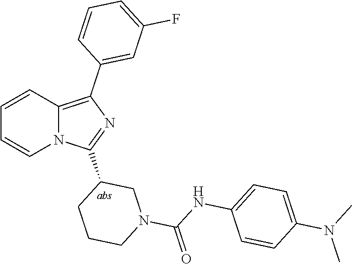 [SMILES: CN(C)c1ccc(NC(=O)N2CCC[C@H](c3nc(-c4cccc(F)c4)c4ccccn34)C2)cc1] | 10-28 | 10.72 | 457.2 | 457.7 | [M + H]⁺ | 2 |
| 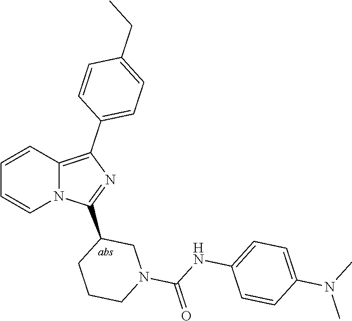 [SMILES: CCc1ccc(-c2nc([C@@H]3CCCN(C(=O)Nc4ccc(N(C)C)cc4)C3)n3ccccc23)cc1] | 10-29 | 10.21 | 467.3 | 467.9 | [M + H]⁺ | 2 |
| 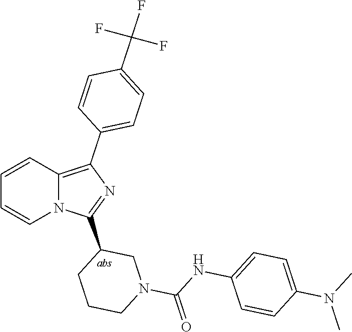 [SMILES: CN(C)c1ccc(NC(=O)N2CCC[C@@H](c3nc(-c4ccc(C(F)(F)F)cc4)c4ccccn34)C2)cc1] | 10-30 | 12.07 | 507.2 | 507.6 | [M + H]⁺ | 2 |
| 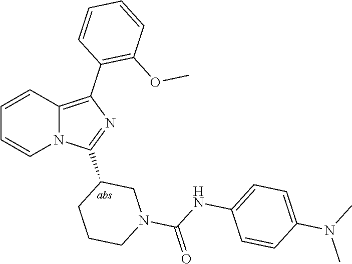 [SMILES: COc1ccccc1-c1nc([C@H]2CCCN(C(=O)Nc3ccc(N(C)C)cc3)C2)n2ccccc12] | 10-31 | 9.06 | 469.2 | 470 | [M + H]⁺ | 2 |
| 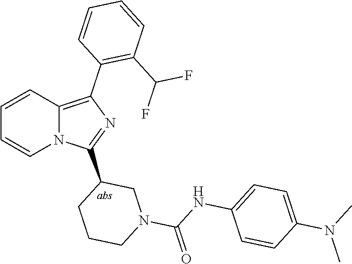 [SMILES: CN(C)c1ccc(NC(=O)N2CCC[C@@H](c3nc(-c4ccccc4C(F)F)c4ccccn34)C2)cc1] | 10-32 | 10.86 | 489.2 | 489.5 | [M + H]⁺ | 2 |
| 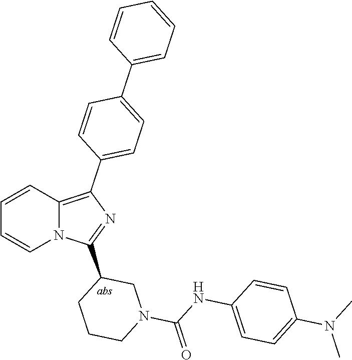 [SMILES: CN(C)c1ccc(NC(=O)N2CCC[C@@H](c3nc(-c4ccc(-c5ccccc5)cc4)c4ccccn34)C2)cc1] | 10-33 | 11.8 | 515.3 | 515.9 | [M + H]⁺ | 2 |
| 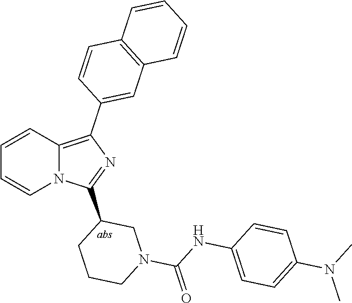 [SMILES: CN(C)c1ccc(NC(=O)N2CCC[C@@H](c3nc(-c4ccc5ccccc5c4)c4ccccn34)C2)cc1] | 10-34 | 11.45 | 489.3 | 489.6 | [M + H]⁺ | 2 |
| 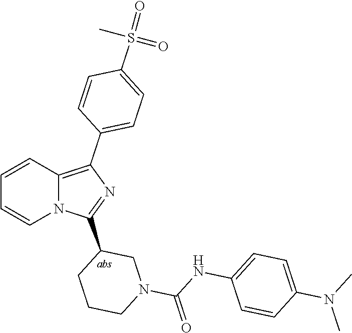 [SMILES: CN(C)c1ccc(NC(=O)N2CCC[C@@H](c3nc(-c4ccc(S(C)(=O)=O)cc4)c4ccccn34)C2)cc1] | 10-35 | 9.6 | 517.2 | 517.9 | [M + H]⁺ | 2 |
| 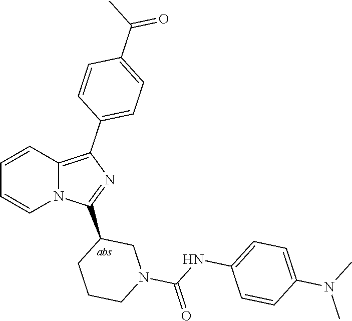 [SMILES: CC(=O)c1ccc(-c2nc([C@@H]3CCCN(C(=O)Nc4ccc(N(C)C)cc4)C3)n3ccccc23)cc1] | 10-36 | 10.15 | 481.2 | 482.2 | [M + H]⁺ | 2 |
| 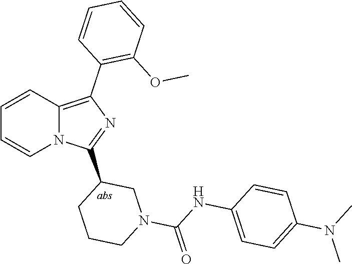 [SMILES: COc1ccccc1-c1nc([C@@H]2CCCN(C(=O)Nc3ccc(N(C)C)cc3)C2)n2ccccc12] | 10-37 | 9.14 | 469.2 | 470 | [M + H]⁺ | 2 |
| 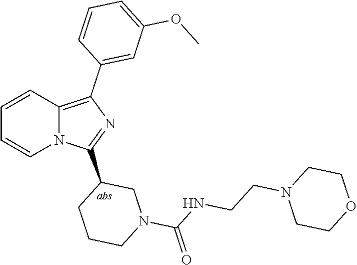 [SMILES: COc1cccc(-c2nc([C@@H]3CCCN(C(=O)NCCN4CCOCC4)C3)n3ccccc23)c1] | 10-38 | 9.59 | 463.3 | 463.7 | [M + H]⁺ | 2 |
| 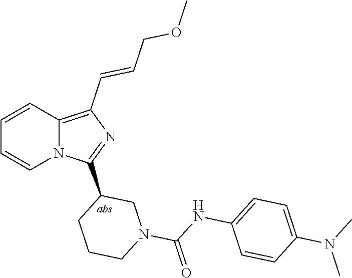 [SMILES: COC/C=C/c1nc([C@@H]2CCCN(C(=O)Nc3ccc(N(C)C)cc3)C2)n2ccccc12] | 10-39 | 8.3 | 433.2 | 433.8 | [M + H]⁺ | 2 |
| 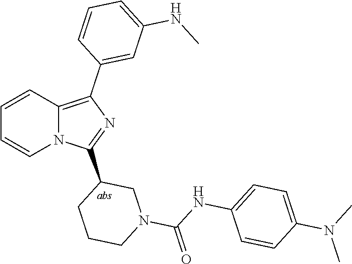 [SMILES: CNc1cccc(-c2nc([C@@H]3CCCN(C(=O)Nc4ccc(N(C)C)cc4)C3)n3ccccc23)c1] | 10-40 | 8.28 | 468.3 | 468.9 | [M + H]⁺ | 2 |
| 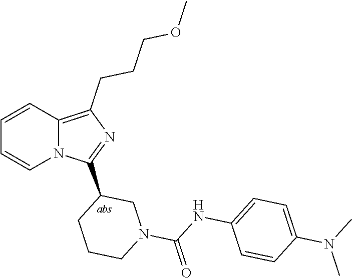 [SMILES: COCCCc1nc([C@@H]2CCCN(C(=O)Nc3ccc(N(C)C)cc3)C2)n2ccccc12] | 10-41 | 7.78 | 435.3 | 435.7 | [M + H]⁺ | 2 |
| 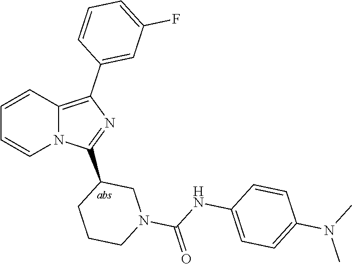 [SMILES: CN(C)c1ccc(NC(=O)N2CCC[C@@H](c3nc(-c4cccc(F)c4)c4ccccn34)C2)cc1] | 10-42 | 10.55 | 457.2 | 457.8 | [M + H]⁺ | 2 |
| 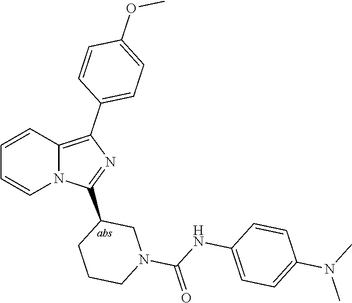 [SMILES: COc1ccc(-c2nc([C@@H]3CCCN(C(=O)Nc4ccc(N(C)C)cc4)C3)n3ccccc23)cc1] | 10-43 | 9.62 | 469.2 | 469.8 | [M + H]⁺ | 2 |
| 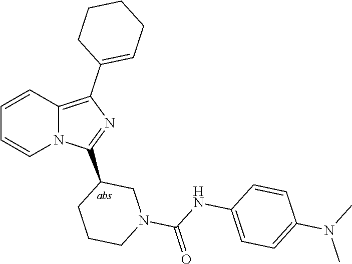 [SMILES: CN(C)c1ccc(NC(=O)N2CCC[C@@H](c3nc(C4=CCCCC4)c4ccccn34)C2)cc1] | 10-44 | 9.47 | 443.3 | 443.8 | [M + H]⁺ | 2 |
| 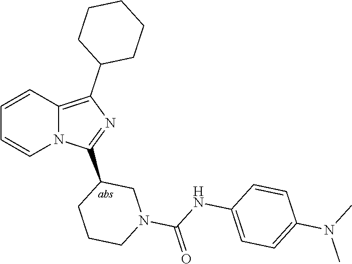 [SMILES: CN(C)c1ccc(NC(=O)N2CCC[C@@H](c3nc(C4CCCCC4)c4ccccn34)C2)cc1] | 10-45 | 8.35 | 445.3 | 445.7 | [M + H]⁺ | 2 |
| 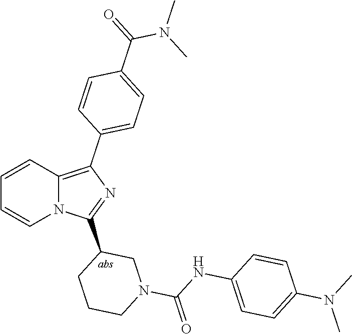 [SMILES: CN(C)C(=O)c1ccc(-c2nc([C@@H]3CCCN(C(=O)Nc4ccc(N(C)C)cc4)C3)n3ccccc23)cc1] | 10-46 | 9.14 | 510.3 | 511 | [M + H]⁺ | 2 |
| 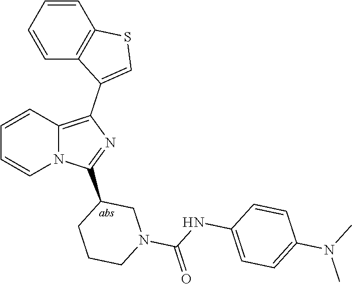 [SMILES: CN(C)c1ccc(NC(=O)N2CCC[C@@H](c3nc(-c4csc5ccccc45)c4ccccn34)C2)cc1] | 10-47 | 11.18 | 495.2 | 496 | [M + H]⁺ | 2 |
| 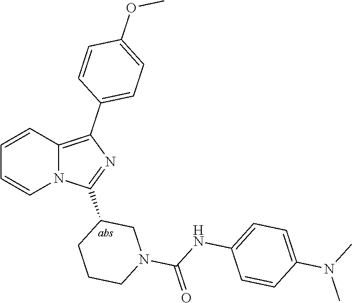 [SMILES: COc1ccc(-c2nc([C@H]3CCCN(C(=O)Nc4ccc(N(C)C)cc4)C3)n3ccccc23)cc1] | 10-48 | 9.43 | 469.2 | 469.8 | [M + H]⁺ | 2 |
| 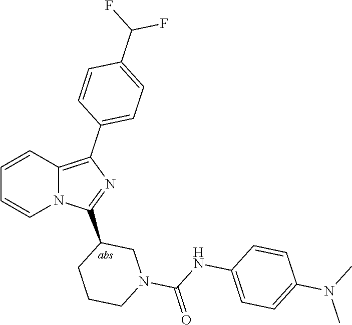 [SMILES: CN(C)c1ccc(NC(=O)N2CCC[C@@H](c3nc(-c4ccc(C(F)F)cc4)c4ccccn34)C2)cc1] | 10-49 | 9.94 | 489.2 | 490.2 | [M + H]⁺ | 2 |
| 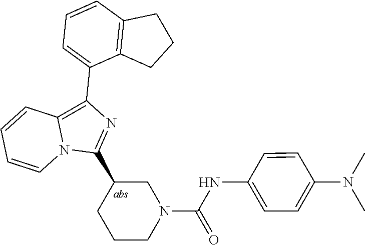 [SMILES: CN(C)c1ccc(NC(=O)N2CCC[C@@H](c3nc(-c4cccc5c4CCC5)c4ccccn34)C2)cc1] | 10-50 | 10.44 | 479.3 | 479.8 | [M + H]⁺ | 2 |
| 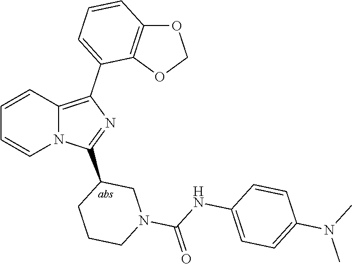 [SMILES: CN(C)c1ccc(NC(=O)N2CCC[C@@H](c3nc(-c4cccc5c4OCO5)c4ccccn34)C2)cc1] | 10-51 | 9.69 | 483.2 | 483.9 | [M + H]⁺ | 2 |
| 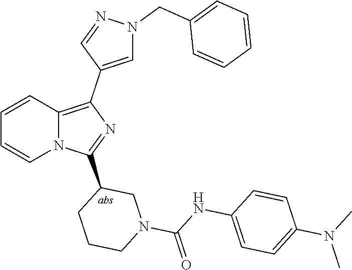 [SMILES: CN(C)c1ccc(NC(=O)N2CCC[C@@H](c3nc(-c4cnn(Cc5ccccc5)c4)c4ccccn34)C2)cc1] | 10-52 | 9.25 | 519.3 | 519.6 | [M + H]⁺ | 2 |
| 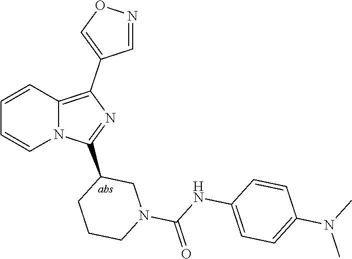 [SMILES: CN(C)c1ccc(NC(=O)N2CCC[C@@H](c3nc(-c4cnoc4)c4ccccn34)C2)cc1] | 10-53 | 9.06 | 430.2 | 430.8 | [M + H]⁺ | 2 |
| 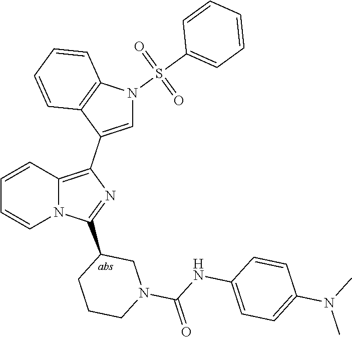 [SMILES: CN(C)c1ccc(NC(=O)N2CCC[C@@H](c3nc(-c4cn(S(=O)(=O)c5ccccc5)c5ccccc45)c4ccccn34)C2)cc1] | 10-54 | 12.24 | 618.2 | 618.8 | [M + H]⁺ | 2 |
| 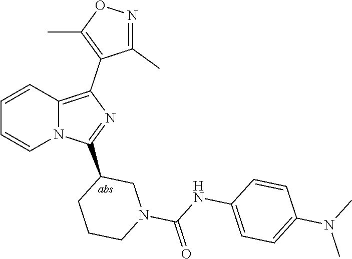 [SMILES: Cc1noc(C)c1-c1nc([C@@H]2CCCN(C(=O)Nc3ccc(N(C)C)cc3)C2)n2ccccc12] | 10-55 | 8.15 | 458.2 | 458.5 | [M + H]⁺ | 2 |
| 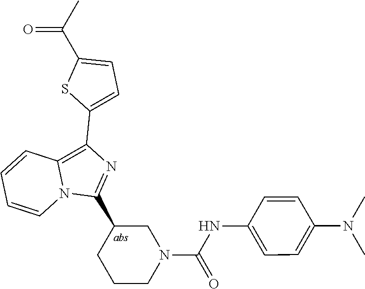 | 10-56 | 10.85 | 459.2 | 459.5 | [M + H]⁺ | 2 |
| 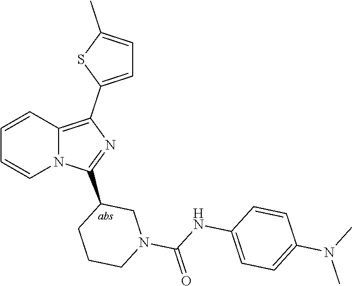 | 10-57 | 10.89 | 487.2 | 487.9 | [M + H]⁺ | 2 |
| 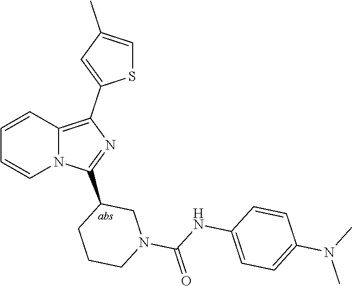 [SMILES: Cc1csc(-c2nc([C@@H]3CCCN(C(=O)Nc4ccc(N(C)C)cc4)C3)n3ccccc23)c1] | 10-58 | 11.11 | 459.2 | 459.7 | [M + H]⁺ | 2 |
| 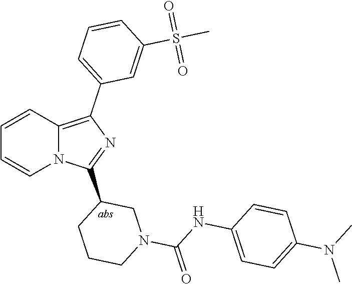 [SMILES: CN(C)c1ccc(NC(=O)N2CCC[C@@H](c3nc(-c4cccc(S(C)(=O)=O)c4)c4ccccn34)C2)cc1] | 10-59 | 9.57 | 517.2 | 517.9 | [M + H]⁺ | 2 |
| 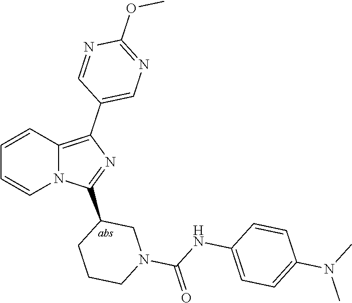 [SMILES: COc1ncc(-c2nc([C@@H]3CCCN(C(=O)Nc4ccc(N(C)C)cc4)C3)n3ccccc23)cn1] | 10-60 | 9.81 | 471.2 | 471.8 | [M + H]⁺ | 2 |
| 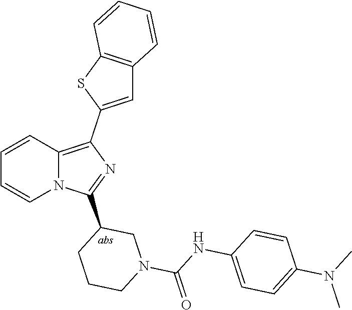 [SMILES: CN(C)c1ccc(NC(=O)N2CCC[C@@H](c3nc(-c4cc5ccccc5s4)c4ccccn34)C2)cc1] | 10-61 | 12.65 | 495.2 | 496.1 | [M + H]⁺ | 2 |
| 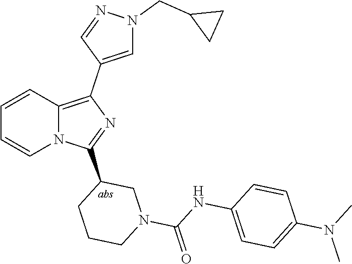 [SMILES: CN(C)c1ccc(NC(=O)N2CCC[C@@H](c3nc(-c4cnn(CC5CC5)c4)c4ccccn34)C2)cc1] | 10-62 | 9.25 | 483.3 | 484.1 | [M + H]⁺ | 2 |
| 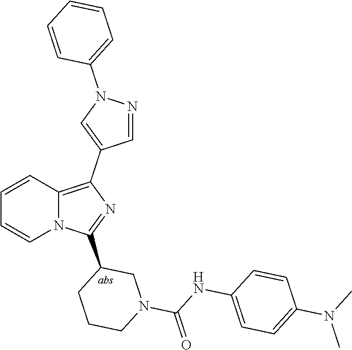 [SMILES: CN(C)c1ccc(NC(=O)N2CCC[C@@H](c3nc(-c4cnn(-c5ccccc5)c4)c4ccccn34)C2)cc1] | 10-63 | 10.2 | 505.3 | 505.8 | [M + H]⁺ | 2 |
| 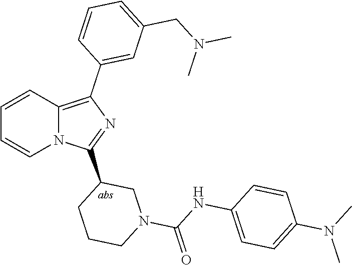 [SMILES: CN(C)Cc1cccc(-c2nc([C@@H]3CCCN(C(=O)Nc4ccc(N(C)C)cc4)C3)n3ccccc23)c1] | 10-64 | 8.16 | 496.3 | 496.8 | [M + H]⁺ | 2 |
| 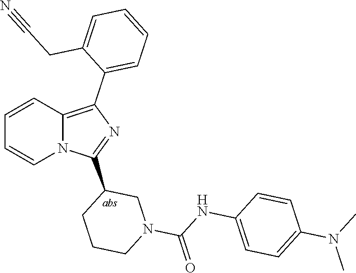 [SMILES: CN(C)c1ccc(NC(=O)N2CCC[C@@H](c3nc(-c4ccccc4CC#N)c4ccccn34)C2)cc1] | 10-65 | 9.7 | 478.2 | 478.7 | [M + H]⁺ | 2 |
| 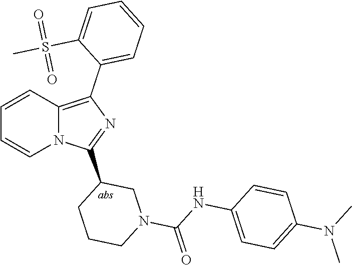 [SMILES: CN(C)c1ccc(NC(=O)N2CCC[C@@H](c3nc(-c4ccccc4S(C)(=O)=O)c4ccccn34)C2)cc1] | 10-66 | 8.89 | 517.2 | 517.7 | [M + H]⁺ | 2 |
| 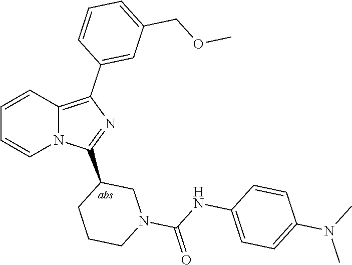 [SMILES: COCc1cccc(-c2nc([C@@H]3CCCN(C(=O)Nc4ccc(N(C)C)cc4)C3)n3ccccc23)c1] | 10-67 | 9.09 | 483.3 | 484 | [M + H]⁺ | 2 |
| 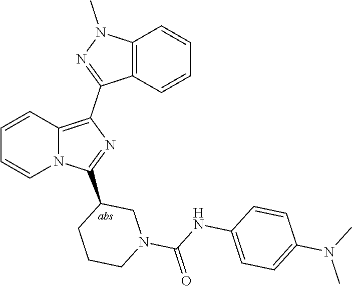 [SMILES: CN(C)c1ccc(NC(=O)N2CCC[C@@H](c3nc(-c4nn(C)c5ccccc45)c4ccccn34)C2)cc1] | 10-68 | 11.53 | 493.3 | 493.9 | [M + H]⁺ | 2 |
| 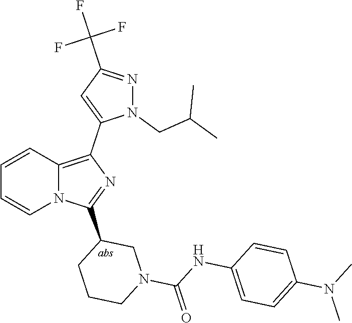 [SMILES: CC(C)Cn1nc(C(F)(F)F)cc1-c1nc([C@@H]2CCCN(C(=O)Nc3ccc(N(C)C)cc3)C2)n2ccccc12] | 10-69 | 12.65 | 553.3 | 553.9 | [M + H]⁺ | 2 |
| 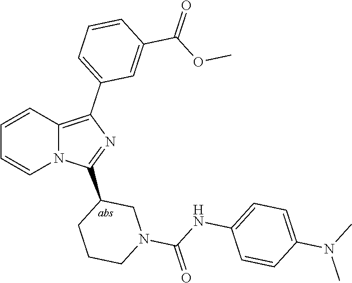 [SMILES: COC(=O)c1cccc(-c2nc([C@@H]3CCCN(C(=O)Nc4ccc(N(C)C)cc4)C3)n3ccccc23)c1] | 10-70 | 10.83 | 497.2 | 497.8 | [M + H]⁺ | 2 |
| 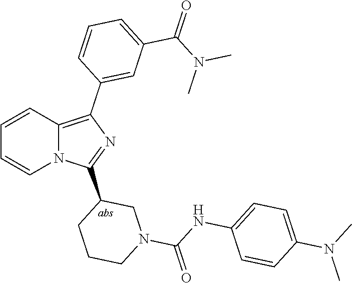 [SMILES: CN(C)C(=O)c1cccc(-c2nc([C@@H]3CCCN(C(=O)Nc4ccc(N(C)C)cc4)C3)n3ccccc23)c1] | 10-71 | 9.15 | 510.3 | 511.1 | [M + H]⁺ | 2 |
| 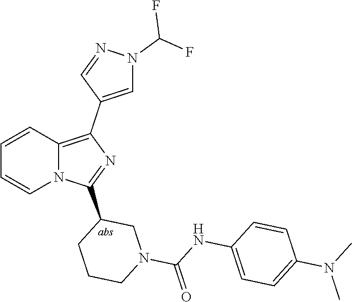 [SMILES: CN(C)c1ccc(NC(=O)N2CCC[C@@H](c3nc(-c4cnn(C(F)F)c4)c4ccccn34)C2)cc1] | 10-72 | 9.05 | 479.2 | 479.7 | [M + H]⁺ | 2 |

### Example 11

### Synthesis of Compound 11-9 and Other Representative Compounds

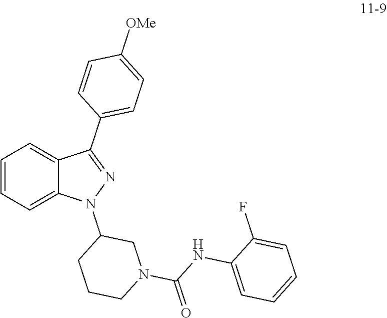 [SMILES: COc1ccc(-c2nn(C3CCCN(C(=O)Nc4ccccc4F)C3)c3ccccc23)cc1]

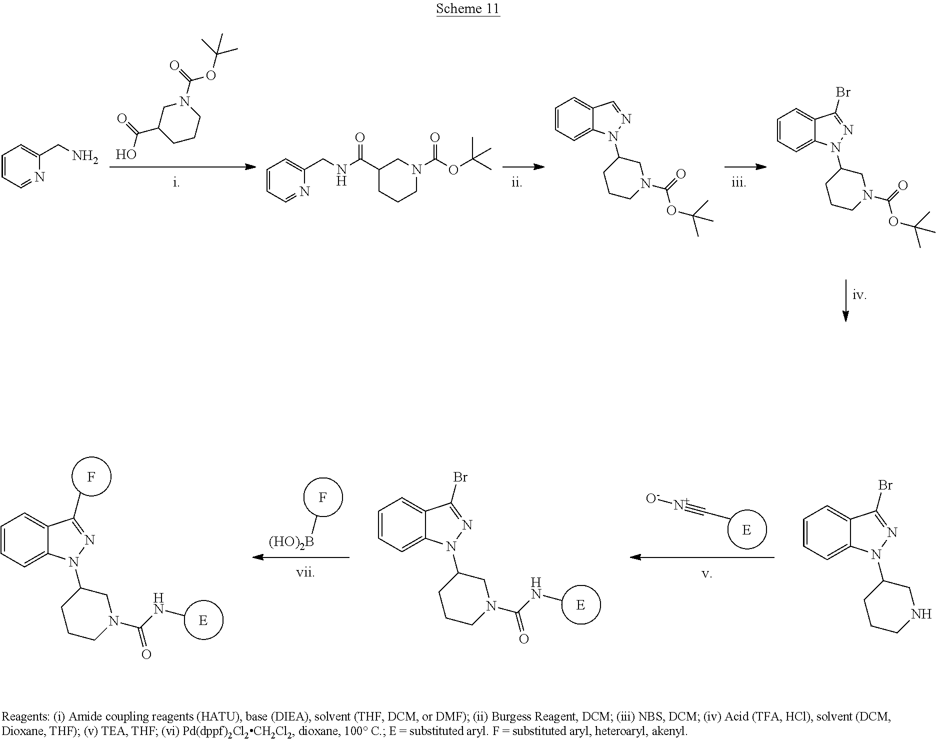

### Step 11-1. Synthesis of tert-butyl (R)-3-((pyridin-2-ylmethyl)carbamoyl)piperidine-1-carboxylate (INT 11A)

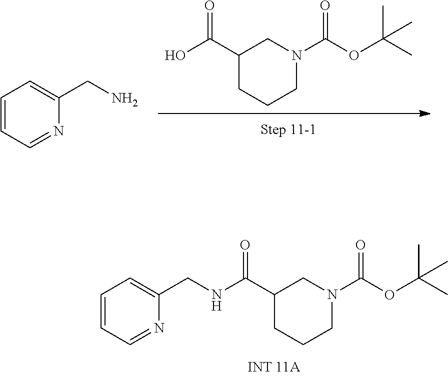 [SMILES: NCc1ccccn1 ; CC(C)(C)OC(=O)N1CCCC(C(=O)O)C1 ; CC(C)(C)OC(=O)N1CCCC(C(=O)NCc2ccccn2)C1]

1-(tert-butoxycarbonyl)piperidine-3-carboxylic acid (12.72 g, 55.48 mmol), DIEA (11.95 g, 92.5 mmol) and HATU (21.1 g, 55.5 mmol) were added to a flask containing DCM (50 mL) and DMF (5 mL). After 5 min, 2-pyridylmethanamine (4.72 mL, 46.2 mmol) was added. After stirring at 25° C. for 16 hr, the reaction mixture was concentrated, diluted with H₂O, and extracted with EA (2×). The combined organic extracts were washed with saturated Na₂CO₃ (aq) and brine, then dried (Na₂SO₄), filtered, and concentrated to provide a crude residue that was purified by SiO₂ chromatography (EA/petroleum ether) to provide 14.7 g (99%) of tert-butyl 3-((pyridin-2-ylmethyl)carbamoyl) piperidine-1-carboxylate (INT 11A). LCMS-ESI (m/z) calculated for C₁₇H₂₅N₃O₃: 319.4; found 320.2 [M+H]⁺, t_{R}=0.72 min (Method 4). ¹H NMR (400 MHz, DMSO-d6): δ=8.53 (br t, J=5.6 Hz, 1H), 8.49 (dd, J=0.6, 4.8 Hz, 1H), 7.76 (dt, J=1.8, 7.7 Hz, 1H), 7.32-7.18 (m, 2H), 4.34 (br s, 2H), 3.73-3.50 (m, 2H), 3.14 (dq, J=4.3, 7.3 Hz, 2H), 2.69 (s, 3H), 2.33 (tt, J=3.8, 11.2 Hz, 1H), 1.88 (br d, J=11.5 Hz, 1H), 1.71-1.49 (m, 2H), 1.39 (s, 9H), 1.37-1.29 (m, 1H).

### Step 11-2. Synthesis of tert-butyl 3-(1H-indazol-1-yl)piperidine-1-carboxylate (INT 11B)

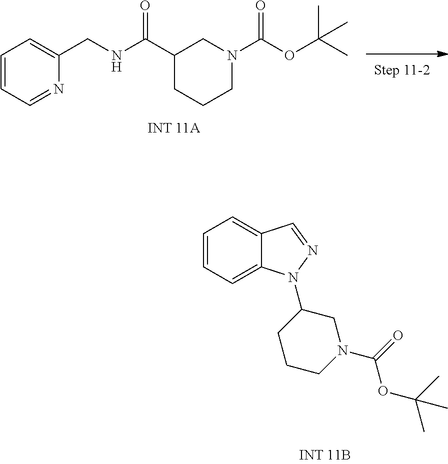 [SMILES: CC(C)(C)OC(=O)N1CCCC(C(=O)NCc2ccccn2)C1 ; CC(C)(C)OC(=O)N1CCCC(n2ncc3ccccc32)C1]

Burgess reagent (11.49 g, 48.22 mmol) was added to a vial containing INT 11A (13 g, 43.1 mmol) in DCM (140 ml). After stirring for 2 hr at 25° C., H₂O was added, and the reaction mixture was extracted with DCM (2×). The combined organic layers were washed with brine, dried (Na₂SO₄), concentrated, and purified by SiO₂ chromatography (MeOH/DCM) to afford 13 g (98%) of tert-butyl 3-(1H-indazol-1-yl)piperidine-1-carboxylate (INT 11B) as a yellow oil. LCMS-ESI (m/z) calculated for C₁₇H₂₃N₃O₂: 301.4; found 302.1 [M+H]⁺, t_{R}=1.09 min (Method 10).

### Step 11-3. Synthesis of tert-butyl (3-(3-bromo-1H-indazol-1-yl)piperidine-1-carboxylate (INT 11C)

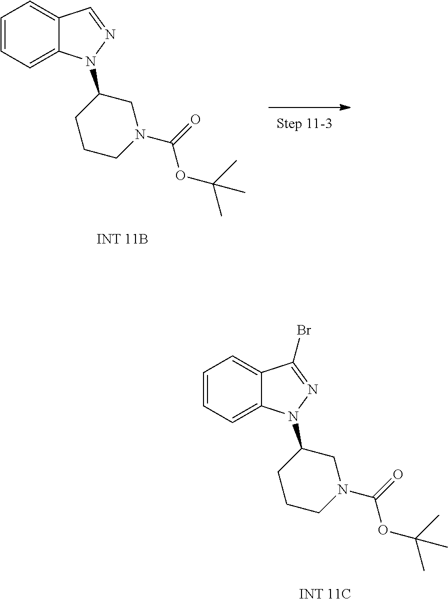 [SMILES: CC(C)(C)OC(=O)N1CCC[C@@H](n2ncc3ccccc32)C1 ; CC(C)(C)OC(=O)N1CCC[C@@H](n2nc(Br)c3ccccc32)C1]

NBS (2.95 g, 16.6 mmol) was added to a stirring solution of INT 11B (5 g, 16.6 mmol) in DMF (50 mL) at 25° C. After stirring for 1 h, the reaction mixture was diluted with H₂O, washed with brine, dried (Na₂SO₄), filtered, concentrated and purified by SiO₂ chromatography (EA/petroleum ether) to afford 2.4 g (37%) of tert-butyl 3-(3-bromo-1H-indazol-1-yl)piperidine-1-carboxylate (INT 11C) as a yellow oil. LCMS-ESI (m/z) calculated for C₁₇H₂₂BrN₃O₂: 380.2; found 382.1 [M+H]⁺, t_{R}=1.00 min (Method 4). ¹H NMR (400 MHZ, CDCl₃): δ=7.86 (br s, 1H), 7.35 (br d, J=9.3 Hz, 1H), 6.74 (dd, J=6.4, 9.1 Hz, 1H), 6.65-6.54 (m, 1H), 4.48-4.10 (m, 2H), 3.18-2.95 (m, 2H), 2.93-2.63 (m, 2H), 2.21-2.12 (m, 1H), 1.87-1.74 (m, 1H), 1.67-1.53 (m, 1H), 1.47 (s, 9H).

### Step 11-4. Synthesis of 3-bromo-1-(piperidin-3-yl)-1H-indazole (INT 11D)

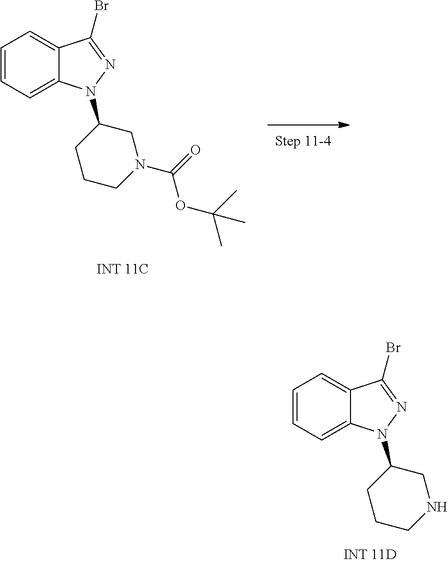 [SMILES: CC(C)(C)OC(=O)N1CCC[C@@H](n2nc(Br)c3ccccc32)C1 ; Brc1nn([C@@H]2CCCNC2)c2ccccc12]

A solution of INT 11C (2 g, 5.26 mmol) was dissolved in 4 M HCl/Dioxane (16.67 mL). After stirring for 1 hr at 25° C., the reaction mixture was concentrated to afford 1.6 g (99%) of (3-bromo-1-(piperidin-3-yl)-1H-indazole (INT 11D) as a yellow solid that was used in the next step without further purification. LCMS-ESI (m/z) calculated for C₁₂H₁₄BrN₃: 280.2; found 280.1 [M+H]⁺, t_{R}=0.710 min (Method 4).

### Step 11-5. Synthesis of 3-(3-bromo-1H-indazol-1-yl)-N-(4-(dimethylamino) phenyl) piperidine-1-carboxamide (INT 11E)

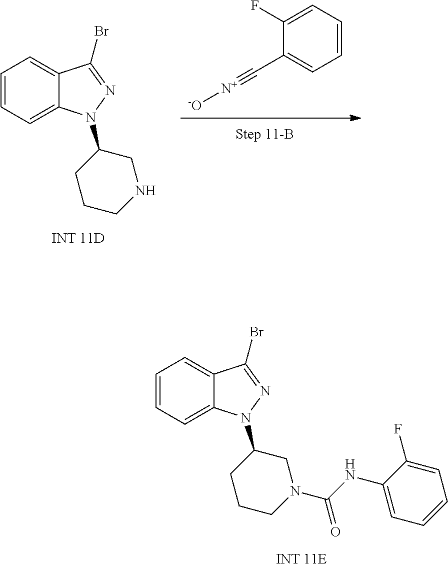

To a solution of INT 11D (1.6 g, 5.05 mmol) in THF (20 mL) were added TEA (1.41 mL, 10.11 mmol) and 2-fluorobenzonitrile oxide (831.44 mg, 6.06 mmol) at 25° C. The resulting mixture was stirred at 25° C. for 2 hr. The mixture was concentrated and purified by SiO₂ chromatography (10% MeOH in EA/petroleum ether) to afford 1.8 g (85%) of 3-(3-bromo-1H-indazol-1-yl)-N-(4-(dimethylamino) phenyl)piperidine-1-carboxamide (INT 11E) as a yellow solid. LCMS-ESI (m/z) calculated for C₁₉H₁₈BrFN₄O: 417.2; found 419.0 [M+H]⁺, t_{R} 0.927 min (Method 4). ¹H NMR (400 MHZ, CDCl₃): δ=8.07 (dt, J=1.5, 8.2 Hz, 1H), 7.93 (d, J=7.1 Hz, 1H), 7.37 (d, J=9.3 Hz, 1H), 7.18-7.04 (m, 2H), 7.04-6.94 (m, 2H), 6.77 (dd, J=6.4, 9.3 Hz, 1H), 6.64 (t, J=6.4 Hz, 1H), 4.36 (br d, J=9.5 Hz, 1H), 3.90 (br d, J=13.5 Hz, 1H), 3.37-3.18 (m, 3H), 2.31-2.14 (m, 2H), 1.95-1.83 (m, 1H), 1.81-1.67 (m, 1H).

### Step 11-6. Synthesis of (N-(4-(dimethylamino)phenyl)-3-(3-(2-(trifluoromethoxy) phenyl)-1H-indazol-1-yl)piperidine-1-carboxamide (Compound 11-9)

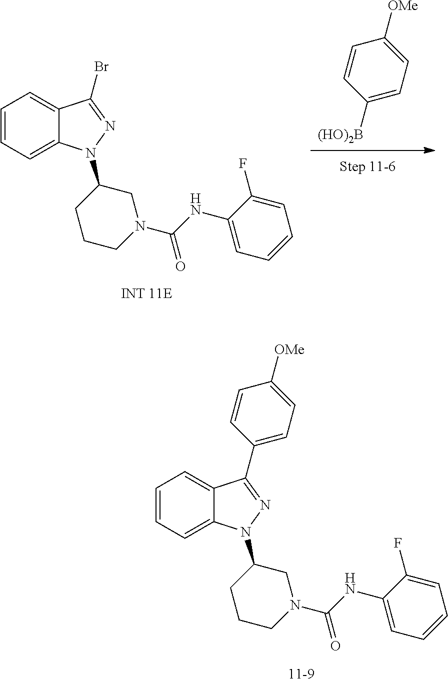 [SMILES: O=C(Nc1ccccc1F)N1CCC[C@@H](n2nc(Br)c3ccccc32)C1 ; COc1ccc(B(O)O)cc1 ; COc1ccc(-c2nn([C@@H]3CCCN(C(=O)Nc4ccccc4F)C3)c3ccccc23)cc1]

INT 11E (100 mg, 0.24 mmol), (4-methoxyphenyl)boronic acid (72.8 mg, 0.48 mmol), Pd(dppf)Cl₂ (17.5 mg, 0.02 mmol) and Cs₂CO₃ (156.2 mg, 0.48 mmol) were dissolved in dioxane (2.5 mL) and H₂O (0.5 mL). The tube was purged with N₂, sealed, and heated at 100° C. for 6 h under N₂. The mixture was cooled, diluted with H₂O, and extracted with EA (2×). The combined organic layers were washed with brine (2×), then dried (Na₂SO₂), filtered, and purified by prep HPLC chromatography (H₂O (0.225% FA)/ACN). The appropriate fractions were combined, concentrated, and lyophilized to afford 32.4 mg (30%) of N-(4-(dimethylamino)phenyl)-3-(3-(2-(trifluoromethoxy)phenyl)-1H-indazol-1-yl)piperidine-1-carboxamide (Compound 11-9). LCMS-ESI (m/z) calculated for C26H₂₅F₃N₄O₂: 444.5; found 445.2 [M+H]⁺, t_{R}=1.61 min (Method 8). ¹H NMR (400 MHZ, CD₃OD) J=8.21 (d, J=8.0 Hz, 1H), 7.77-7.68 (m, 3H), 7.53-7.45 (m, 1H), 7.19-7.10 (m, 3H), 7.04 (d, J=8.0 Hz, 2H), 6.82 (dd, J=8.0 Hz, 1H), 6.71 (t, J=8.0 Hz, 1H), 4.41 (br d, J=12.0 Hz, 1H), 4.22 (br d, J=12.0 Hz, 1H), 3.86 (s, 3H), 3.45 (tt, J=4.0, 12.0 Hz, 1H), 3.37 (s, 1H), 3.23-3.13 (m, 1H), 2.29-2.21 (m, 1H), 2.19-2.07 (m, 1H), 1.98-1.78 (m, 2H).

The compounds listed in the Table 11 was made using the procedures of Scheme 11.

TABLE 11

| | | Purity | | | | |
|---|---|---|---|---|---|---|
| | Cpd | RT | | Observed | | Purity |
| Structure | No. | (min) | MW | m/z | Ion | Method |
| 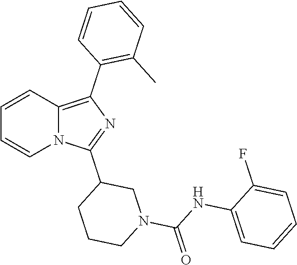 [SMILES: Cc1ccccc1-c1nc(C2CCCN(C(=O)Nc3ccccc3F)C2)n2ccccc12] | 11-1 | 1.623 | 428.2 | 429.3 | [M + H]⁺ | 9 |
| 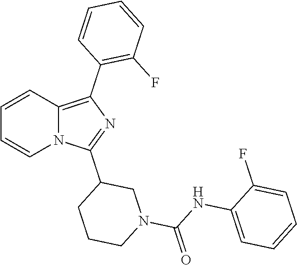 [SMILES: O=C(Nc1ccccc1F)N1CCCC(c2nc(-c3ccccc3F)c3ccccn23)C1] | 11-2 | 1.735 | 432.2 | 433.2 | [M + H]⁺ | 9 |
| 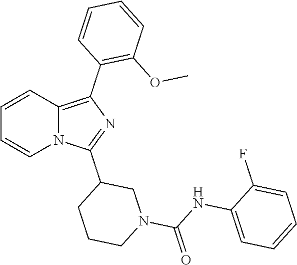 [SMILES: COc1ccccc1-c1nc(C2CCCN(C(=O)Nc3ccccc3F)C2)n2ccccc12] | 11-3 | 1.558 | 444.2 | 445.2 | [M + H]⁺ | 9 |
| 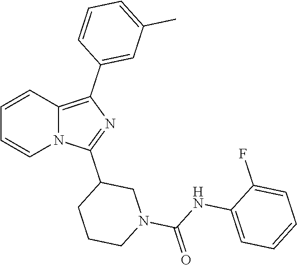 [SMILES: Cc1cccc(-c2nc(C3CCCN(C(=O)Nc4ccccc4F)C3)n3ccccc23)c1] | 11-4 | 1.78 | 428.2 | 429.3 | [M + H]⁺ | 8 |
| 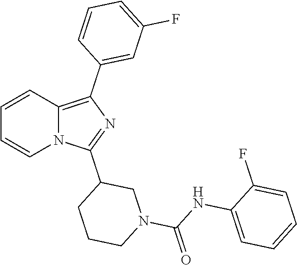 [SMILES: O=C(Nc1ccccc1F)N1CCCC(c2nc(-c3cccc(F)c3)c3ccccn23)C1] | 11-5 | 1.897 | 432.2 | 433.3 | [M + H]⁺ | 8 |
| 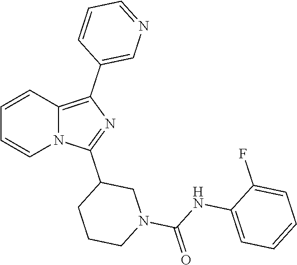 [SMILES: O=C(Nc1ccccc1F)N1CCCC(c2nc(-c3cccnc3)c3ccccn23)C1] | 11-6 | 1.275 | 415.2 | 416.2 | [M + H]⁺ | 8 |
| 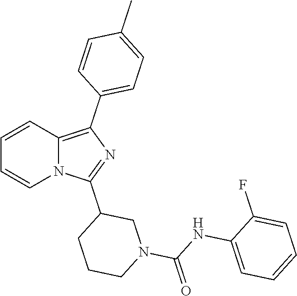 [SMILES: Cc1ccc(-c2nc(C3CCCN(C(=O)Nc4ccccc4F)C3)n3ccccc23)cc1] | 11-7 | 1.736 | 428.2 | 429.2 | [M + H]⁺ | 8 |
| 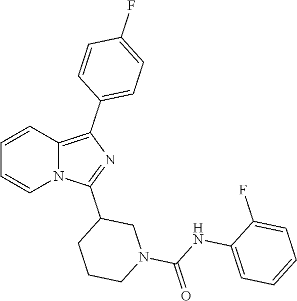 [SMILES: O=C(Nc1ccccc1F)N1CCCC(c2nc(-c3ccc(F)cc3)c3ccccn23)C1] | 11-8 | 1.755 | 432.2 | 433.2 | [M + H]⁺ | 8 |
| 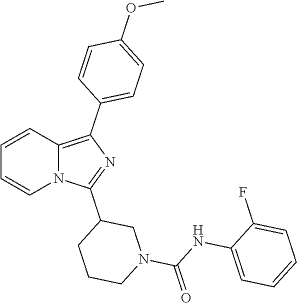 [SMILES: COc1ccc(-c2nc(C3CCCN(C(=O)Nc4ccccc4F)C3)n3ccccc23)cc1] | 11-9 | 1.609 | 444.2 | 445.2 | [M + H]⁺ | 8 |
| 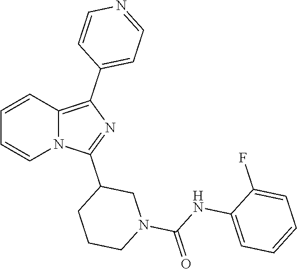 [SMILES: O=C(Nc1ccccc1F)N1CCCC(c2nc(-c3ccncc3)c3ccccn23)C1] | 11-10 | 1.267 | 415.2 | 416.2 | [M + H]⁺ | 8 |
| 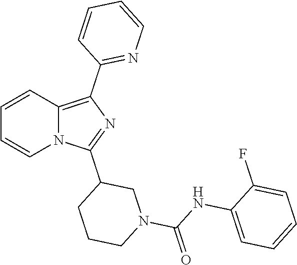 [SMILES: O=C(Nc1ccccc1F)N1CCCC(c2nc(-c3ccccn3)c3ccccn23)C1] | 11-11 | 1.649 | 415.2 | 416.1 | [M + H]⁺ | 8 |

### Example 12

### Synthesis of Compound 12-1 and Other Representative Compounds

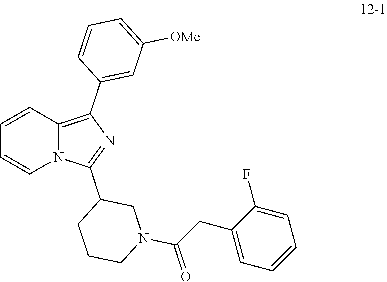 [SMILES: COc1cccc(-c2nc(C3CCCN(C(=O)Cc4ccccc4F)C3)n3ccccc23)c1]

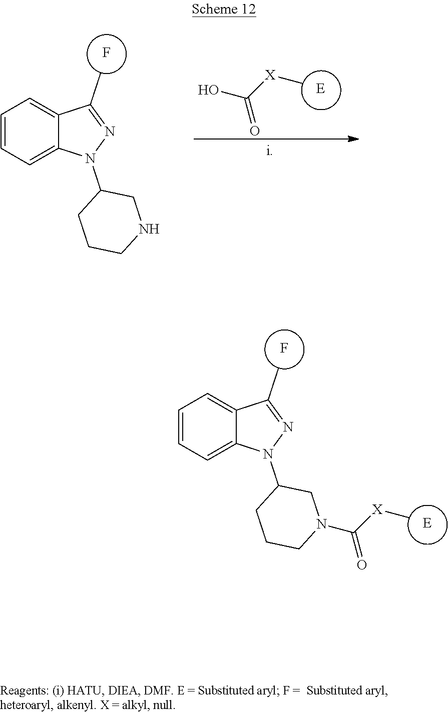

### Step 12-1. Synthesis of 2-(2-fluorophenyl)-1-(3-(1-(3-methoxyphenyl)imidazo[1,5-a]pyridin-3-yl)piperidin-1-yl)ethan-1-one (Compound 12-1)

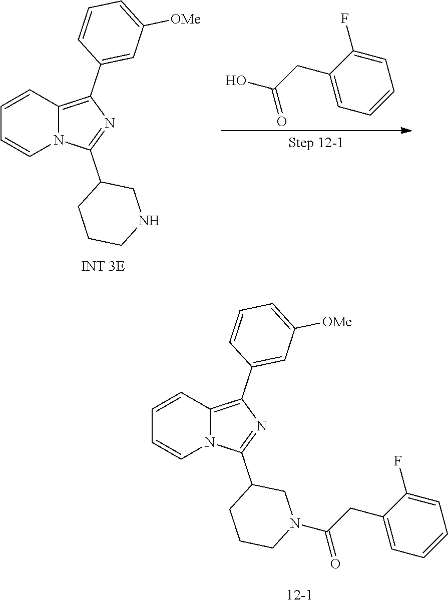 [SMILES: COc1cccc(-c2nc(C3CCCNC3)n3ccccc23)c1 ; O=C(O)Cc1ccccc1F ; COc1cccc(-c2nc(C3CCCN(C(=O)Cc4ccccc4F)C3)n3ccccc23)c1]

To a solution of INT 3E (90 mg, 0.29 mmol) and 2-(2-fluorophenyl)acetic acid (90.3 mg, 0.59 mmol) in DMF (1 mL) were added HATU (167 mg, 0.44 mmol) and DIEA (102 μL, 0.59 mmol). After stirring for 12 h at 25° C., the mixture was concentrated to provide a crude product that was purified by prep-HPLC chromatography (H₂O (0.225% FA)/ACN). The desired fractions were concentrated and lyophilized to provide 9.3 mg (7%) of 2-(2-fluorophenyl)-1-(3-(1-(3-methoxyphenyl)imidazo[1,5-a]pyridin-3-yl)piperidin-1-yl)ethan-1-one (Compound 12-1) as a yellow solid. LCMS-ESI (m/z) calculated for C₂₇H₂₆FN₃O₂: 443.5; found 444.2 [M+H]⁺, t_{R}=1.84 min (Method 8). ¹H NMR (400 MHZ, CD₃OD) δ 8.28 (d, J=7.2 Hz, 1H), 7.90 (br s, 1H), 7.88-7.82 (m, 1H), 7.46-7.29 (m, 5H), 7.22-7.05 (m, 2H), 7.03-6.90 (m, 2H), 6.90-6.74 (m, 1H), 4.82-4.52 (m, 1H), 4.31-4.10 (m, 1H), 3.91-3.87 (m, 4H), 4.00-3.85 (m, 1H), 3.81-3.62 (m, 1H), 3.46 (br t, J=11.0 Hz, 1H), 3.21-2.86 (m, 1H), 2.29-1.94 (m, 3H), 1.81-1.63 (m, 1H).

The compounds listed in Table 12 were made using the procedures of Scheme 12.

TABLE 12

| | | Purity | | | | |
|---|---|---|---|---|---|---|
| | Cpd | RT | | Observed | | Purity |
| Structure | No. | (min) | MW | m/z | Ion | Method |
| 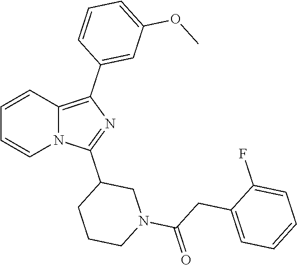 [SMILES: COc1cccc(-c2nc(C3CCCN(C(=O)Cc4ccccc4F)C3)n3ccccc23)c1] | 12-1 | 1.84 | 443.2 | 444.2 | [M + H]⁺ | 8 |
| 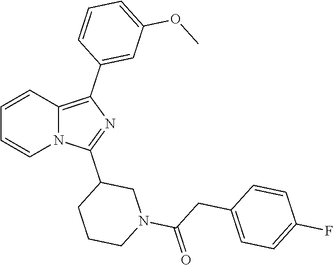 [SMILES: COc1cccc(-c2nc(C3CCCN(C(=O)Cc4ccc(F)cc4)C3)n3ccccc23)c1] | 12-2 | 1.26 | 443.2 | 444.1 | [M + H]⁺ | 8 |
| 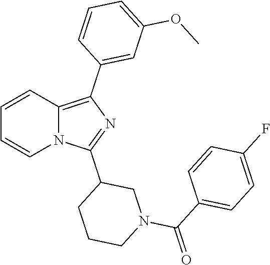 [SMILES: COc1cccc(-c2nc(C3CCCN(C(=O)c4ccc(F)cc4)C3)n3ccccc23)c1] | 12-3 | 2.11 | 429.2 | 430.1 | [M + H]⁺ | 8 |
| 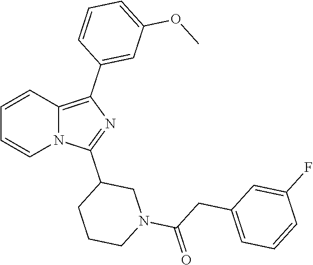 [SMILES: COc1cccc(-c2nc(C3CCCN(C(=O)Cc4cccc(F)c4)C3)n3ccccc23)c1] | 12-4 | 1.658 | 443.2 | 444.2 | [M + H]⁺ | 8 |
| 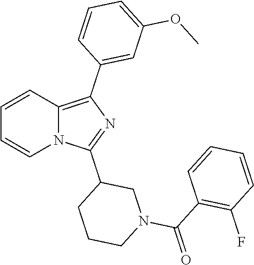 [SMILES: COc1cccc(-c2nc(C3CCCN(C(=O)c4ccccc4F)C3)n3ccccc23)c1] | 12-5 | 2.012 | 429.2 | 430.2 | [M + H]⁺ | 8 |
| 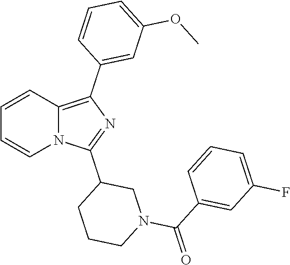 [SMILES: COc1cccc(-c2nc(C3CCCN(C(=O)c4cccc(F)c4)C3)n3ccccc23)c1] | 12-6 | 2.02 | 429.2 | 430.2 | [M + H]⁺ | 8 |
| 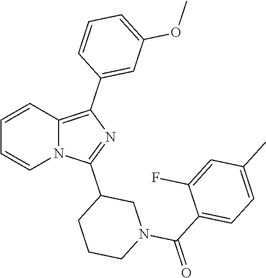 [SMILES: COc1cccc(-c2nc(C3CCCN(C(=O)c4ccc(C)cc4F)C3)n3ccccc23)c1] | 12-7 | 0.855 | 443.2 | 444.2 | [M + H]⁺ | 4 |
| 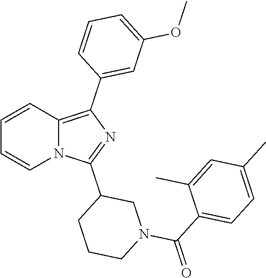 [SMILES: COc1cccc(-c2nc(C3CCCN(C(=O)c4ccc(C)cc4C)C3)n3ccccc23)c1] | 12-8 | 0.866 | 439.2 | 440.2 | [M + H]⁺ | 4 |
| 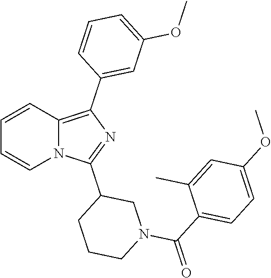 [SMILES: COc1cccc(-c2nc(C3CCCN(C(=O)c4ccc(OC)cc4C)C3)n3ccccc23)c1] | 12-9 | 0.833 | 455.2 | 456.2 | [M + H]⁺ | 4 |
| 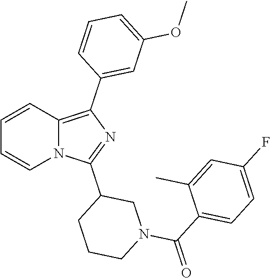 [SMILES: COc1cccc(-c2nc(C3CCCN(C(=O)c4ccc(F)cc4C)C3)n3ccccc23)c1] | 12-10 | 0.849 | 443.2 | 444.2 | [M + H]⁺ | 4 |
| 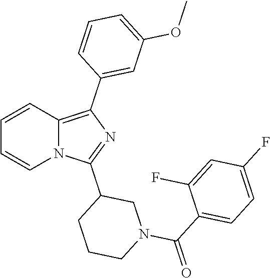 [SMILES: COc1cccc(-c2nc(C3CCCN(C(=O)c4ccc(F)cc4F)C3)n3ccccc23)c1] | 12-11 | 0.842 | 447.2 | 448.2 | [M + H]⁺ | 4 |
| 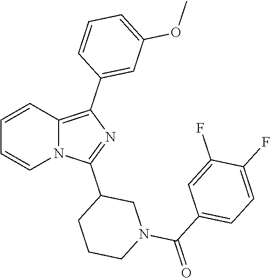 [SMILES: COc1cccc(-c2nc(C3CCCN(C(=O)c4ccc(F)c(F)c4)C3)n3ccccc23)c1] | 12-12 | 0.848 | 447.2 | 448.2 | [M + H]⁺ | 4 |
| 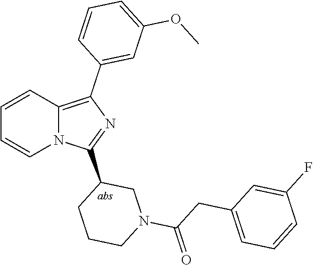 [SMILES: COc1cccc(-c2nc([C@@H]3CCCN(C(=O)Cc4cccc(F)c4)C3)n3ccccc23)c1] | 12-13 | 11.63 | 443.2 | 443.9 | [M + H]⁺ | 2 |
| 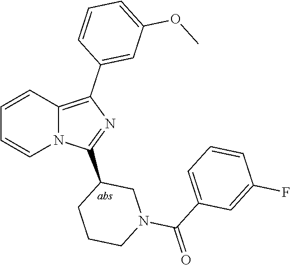 [SMILES: COc1cccc(-c2nc([C@@H]3CCCN(C(=O)c4cccc(F)c4)C3)n3ccccc23)c1] | 12-14 | 11.69 | 429.2 | 430.1 | [M + H]⁺ | 2 |
| 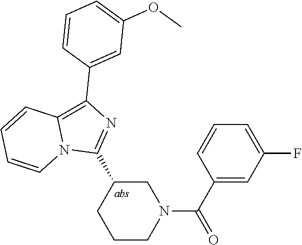 [SMILES: COc1cccc(-c2nc([C@H]3CCCN(C(=O)c4cccc(F)c4)C3)n3ccccc23)c1] | 12-15 | 11.85 | 429.2 | 430 | [M + H]⁺ | 2 |
| 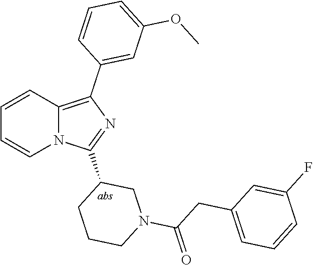 [SMILES: COc1cccc(-c2nc([C@H]3CCCN(C(=O)Cc4cccc(F)c4)C3)n3ccccc23)c1] | 12-16 | 11.99 | 443.2 | 443.9 | [M + H]⁺ | 2 |

### Example 13

### Synthesis of Compound 13-1 and Other Representative Compounds

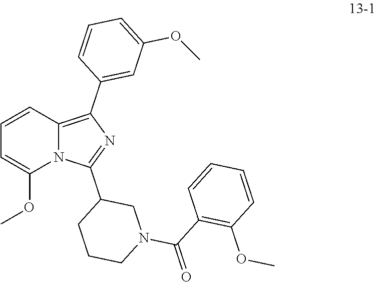

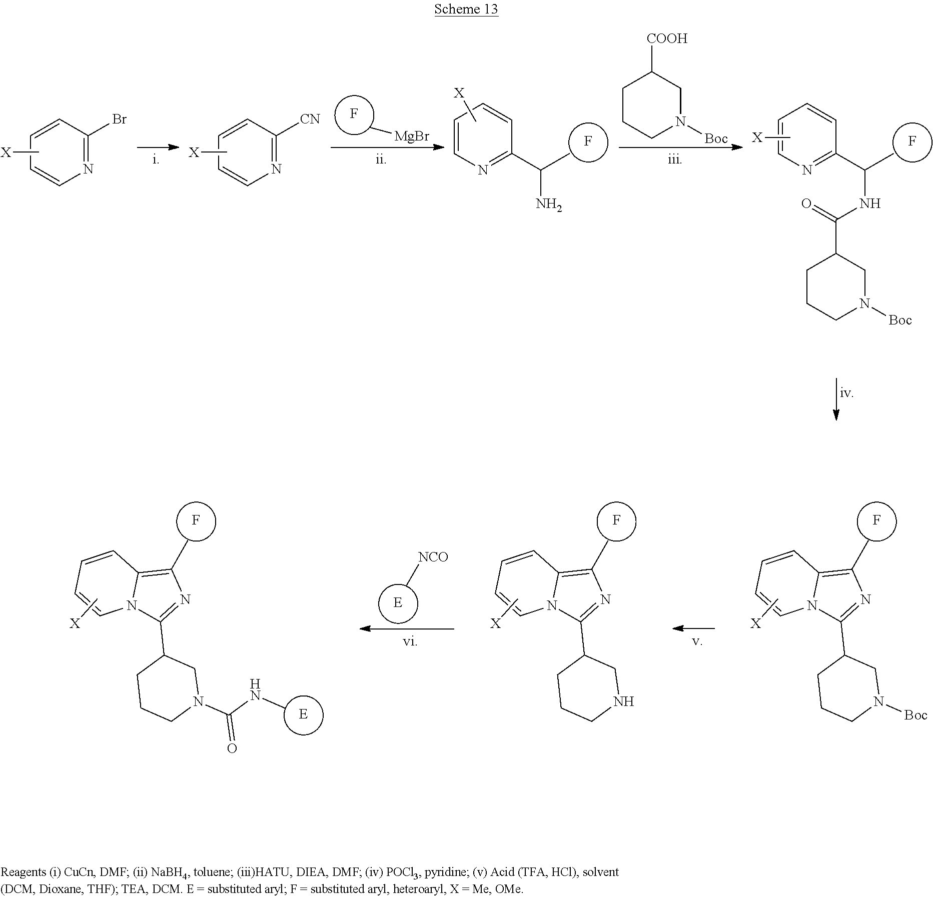

### Step 13-1. Synthesis of 6-methoxypicolinonitrile (INT 13A)

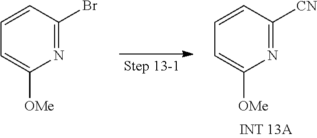 [SMILES: COc1cccc(Br)n1 ; COc1cccc(C#N)n1]

To a stirring solution of 2-bromo-6-methoxy-pyridine (3.27 mL, 26.59 mmol) in DMF (50 mL) was added CuCN (8.71 mL, 39.89 mmol). The reaction mixture was stirred at 140° C. for 12 hr. After cooling to 25° C., the DMF was removed under high vacuum and the residue was diluted with EA (200 mL) and filtered through celite. The resulting solution was washed with H₂O and brine, then dried (Na₂SO₄), filtered and concentrated to give a residue that was triturated with petroleum ether (50 mL) to provide 2.4 g mg (64%) of 6-methoxypicolinonitrile (INT 13A). ¹H NMR (400 MHZ, CDCl₃): 7.66 (t, J=7.87 Hz, 1H) 7.31 (d, J=7.15 Hz, 1H) 6.97 (d, J=8.46 Hz, 1H) 3.97 (s,3 H).

### Step 13-2. Synthesis of (3-methoxyphenyl)(6-methoxypyridin-2-yl)methanamine (INT 13B)

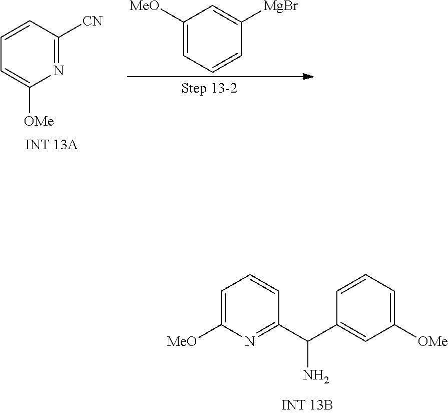

To a stirring solution of INT 13A (800 mg, 5.96 mmol) in toluene (10 mL) was added bromo-(3-methoxyphenyl)magnesium (1 M, 6.56 mL, 6.6 mmol) dropwise under nitrogen at 0° C. After the mixture was stirred at 20° C. for 2 h, MeOH (10 mL) was added and the mixture cooled to 0° C. NaBH₄ (315.9 mg, 8.35 mmol) was added and the mixture was stirred at 20° C. for 12 hrs. The reaction mixture was poured into H₂O (100 mL) and the resulting mixture was filtered. The filtrate was extracted with EA (3×) and the combined organic layers were washed with brine, dried (Na₂SO₄), filtered and concentrated under reduced pressure to give 2.5 g (25%) of (3-methoxyphenyl)(6-methoxypyridin-2-yl)methanamine (INT 13B) as a crude, yellow oil that was used without further purification. LCMS-ESI (m/z) calculated for C₁₄H₁₆N₂O₂: 244.3; found 245.2 [M+H]⁺, t_{R}=0.74 min (Method 4).

### Step 13-3. Synthesis of tert-butyl 3-(((3-methoxyphenyl)(6-methoxypyridin-2-yl)methyl)carbamoyl)piperidine-1-carboxylate (INT 13C)

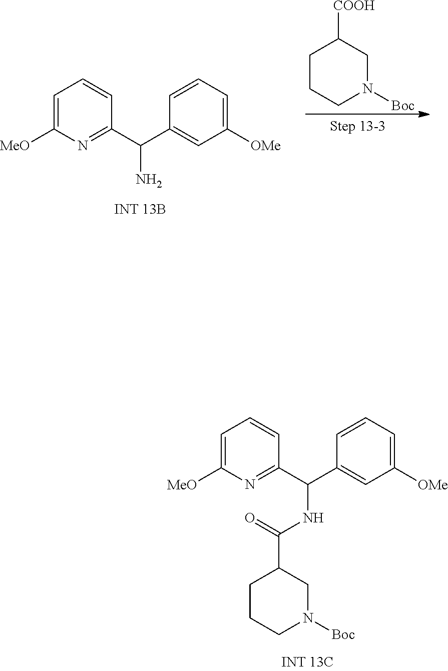 [SMILES: COc1cccc(C(N)c2cccc(OC)n2)c1 ; CC(C)(C)OC(=O)N1CCCC(C(=O)O)C1 ; COc1cccc(C(NC(=O)C2CCCN(C(=O)OC(C)(C)C)C2)c2cccc(OC)n2)c1]

To a stirring solution of INT 13B (2.5 g, 4.4 mmol) in DMF (10 mL) were added DIEA (1.53 mL, 8.80 mmol), HATU (2.01 g, 5.28 mmol) and 1-tert-butoxycarbonylpiperidine-3-carboxylic acid (1.21 g, 5.28 mmol). After the solution was stirred at 20° C. for 2 h, the reaction mixture was poured into H₂O (100 mL) and extracted with EA (3×). The combined organic layers were washed with brine, dried (Na₂SO₄), filtered and concentrated to provide a residue that was purified by SiO₂ chromatography (petroleum ether/EA) to give 600 mg (27%) of tert-butyl 3-(((3-methoxyphenyl)(6-methoxypyridin-2-yl)methyl)carbamoyl)piperidine-1-carboxylate (INT 13C) as a colorless oil. ¹H NMR (400 MHZ, CDCl₃): 7.52 (t, J=7.76 Hz, 1H) 7.34-7.47 (m, 1H) 7.16-7.23 (m, 1H) 6.86-6.98 (m, 2H) 6.83 (d, J=7.34 Hz, 1H) 6.76 (br d, J=8.19 Hz, 1H) 6.64 (d, J=8.31 Hz, 1H) 6.05 (d, J=7.46 Hz, 1H) 4.13-4.20 (m, 1H) 3.99 (d, J=2.08 Hz, 3H) 3.86-3.97 (m, 1H) 3.77 (s, 3H) 2.96-3.09 (m, 2H) 2.73-2.92 (m, 1H) 2.38 (br t, J=10.64 Hz, 1H) 1.99 (br d, J=9.66 Hz, 1H) 1.58-1.86 (m, 3H) 1.44 (d, J=3.79 Hz, 9H).

### Step 13-4. Synthesis of tert-butyl 3-(5-methoxy-1-(3-methoxyphenyl)imidazo[1,5-a]pyridin-3-yl)piperidine-1-carboxylate (INT 13D)

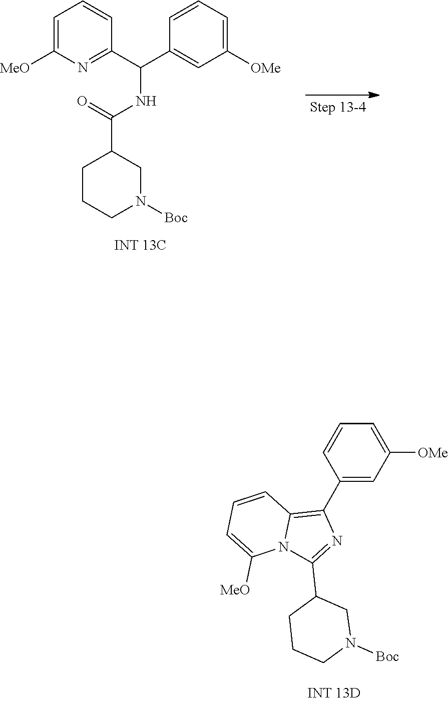 [SMILES: COc1cccc(C(NC(=O)C2CCCN(C(=O)OC(C)(C)C)C2)c2cccc(OC)n2)c1 ; COc1cccc(-c2nc(C3CCCN(C(=O)OC(C)(C)C)C3)n3c(OC)cccc23)c1]

To a stirring solution of INT 13C (600 mg, 1.19 mmol) in pyridine (10 mL) at 0° C. was added POCl₃ (220.3 μL, 2.37 mmol). After the solution was stirred at 20° C. for 2 h, the reaction mixture was concentrated to remove the pyridine. The residue was diluted with EA and washed with 1N HCl (aq) and brine. The organic layer was dried (Na₂SO₄), filtered and concentrated to provide a residue that was purified by SiO₂ chromatography (petroleum ether/EA) to give 200 mg (32%) of tert-butyl 3-(5-methoxy-1-(3-methoxyphenyl)imidazo[1,5-a]pyridin-3-yl)piperidine-1-carboxylate (INT 13D) as a yellow solid. LCMS-ESI (m/z) calculated for C₂₅H₃₁N₃O₄: 437.2; found 438.3 [M+H]⁺, t_{R}=1.156 min (Method 4). ¹H NMR (400 MHZ, CDCl₃): 7.30-7.48 (m, 4H) 6.77-6.85 (m, 1H) 6.70 (dd, J=9.05, 7.09 Hz, 1H) 5.73 (d,J=6.97 Hz, 1H) 4.34-4.70 (m, 1H) 4.14-4.22 (m, 1H) 4.06 (s, 3H) 3.89 (s, 3H) 3.65-3.80 (m, 1H) 2.95-3.32 (m, 1H), 2.83 (br t, J=11.74 Hz, 1H) 2.03-2.34 (m, 2H) 1.83 (br s, 1H) 1.56-1.70 (m, 1H) 1.48 (br s, 9H).

### Step 13-5. Synthesis of 5-methoxy-1-(3-methoxyphenyl)-3-(piperidin-3-yl)imidazo[1,5-a]pyridine (INT 13E)

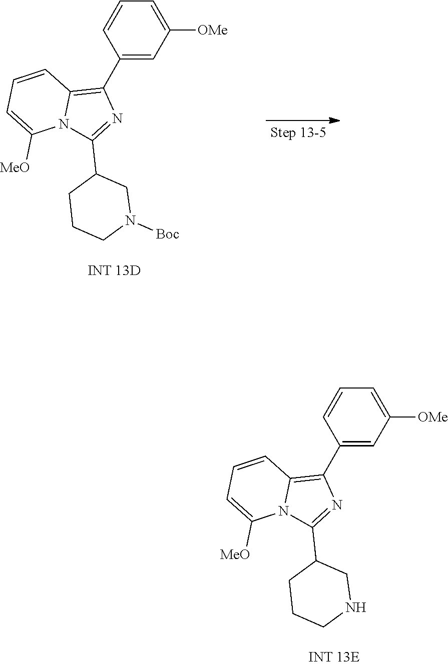 [SMILES: COc1cccc(-c2nc(C3CCCN(C(=O)OC(C)(C)C)C3)n3c(OC)cccc23)c1 ; COc1cccc(-c2nc(C3CCCNC3)n3c(OC)cccc23)c1]

A stirring solution of INT 13D (200 mg, 0.37 mmol) in HCl/MeOH (4 M, 10 mL) was stirred at 20° C. for 2 hrs. The reaction mixture was concentrated and the resulting residue was triturated with THF (5 mL) to give 100 mg (71%) of 5-methoxy-1-(3-methoxyphenyl)-3-(piperidin-3-yl)imidazo[1,5-a]pyridine (INT 13E) as a yellow solid. LCMS-ESI (m/z) calculated for C₂₀H₂₃N₃O₂: 337.1; found 338.2 [M+H]⁺, t_{R}=0.636 min (Method 4). ¹H NMR (400 MHz, C₃DOD): 7.48-7.60 (m, 2H) 7.23-7.37 (m, 3H) 7.12 (dd, J=8.38, 1.77 Hz, 1H) 6.56 (d, J=7.34 Hz, 1H) 4.78 (tt, J=11.98, 3.67 Hz, 1H) 4.28 (s, 3H) 3.91 (s, 3H) 3.85 (br d, J=12.23 Hz, 1H) 3.40-3.59 (m, 2H), 3.15 (td, J=12.75, 3.24 Hz, 1H) 1.99-2.45 (m, 4H).

### Step 13-6. Synthesis of N-(2-fluorophenyl)-3-(5-methoxy-1-(3-methoxyphenyl) imidazo[1,5-a]pyridin-3-yl)piperidine-1-carboxamide (Compound 13-1)

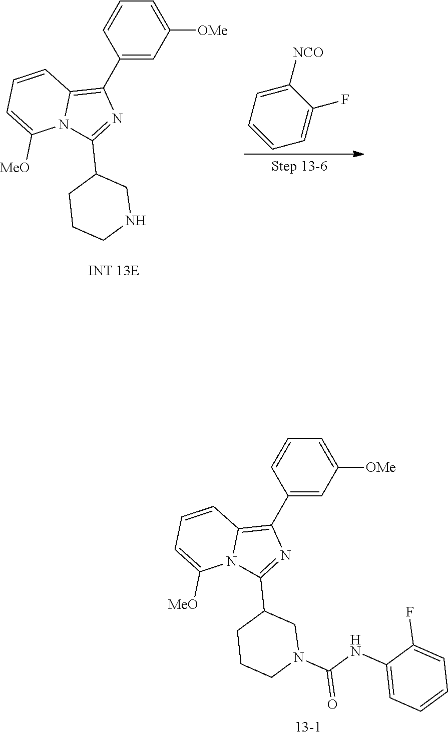

To a stirring solution of INT 13E (100 mg, 0.26 mmol) in DCM (5 mL) at 0° C. were added TEA (111.7 μL, 0.802 mmol) and 1-fluoro-2-isocyanato-benzene (36.67 mg, 0.27 mmol). After stirring at 20° C. for 2 h, the reaction mixture was diluted with DCM and washed with 1N HCl (aq) and brine. The organic layer was dried (Na₂SO₄), filtered and concentrated to provide a residue that was purified by prep TLC chromatography (petroleum ether/EA) to give 100 mg (79%) of N-(2-fluorophenyl)-3-(5-methoxy-1-(3-methoxyphenyl) imidazo[1,5-a] pyridin-3-yl) piperidine -1-carboxamide (Compound 13-1) as a yellow solid. LCMS-ESI (m/z) calculated for C₂₇H₂₇FN₄O₃: 474.5; found 475.2 [M+H]⁺, t_{R}=0.91 min (Method 4). ¹H NMR (400 MHz, DMSO-d₆): 8.34 (s, 1H) 7.39-7.49 (m, 3H) 7.29-7.38 (m, 2H) 7.07-7.21 (m, 3H) 6.78-6.89 (m, 1H) 6.78-6.89 (m, 1H) 6.04 (d, J=7.03 Hz, 1H) 4.49 (br d, J=12.99 Hz, 1H) 4.18 (br d, J=12.64 Hz, 1H) 3.94-4.06 (m, 3H), 3.81 (s, 3H) 3.75 (ddt, J=11.06, 7.45, 3.62, 3.62 Hz, 1 H) 3.09-3.22 (m, 1H) 2.81-2.95 (m, 1H) 2.21 (br d, J=11.68 Hz, 1H), 1.71-2.02 (m, 2H) 1.50-1.67 (m, 1H).

The compound listed in Table 13 was made using the procedures of Scheme 13.

TABLE 13

| | | Purity | | | | |
|---|---|---|---|---|---|---|
| | Cpd | RT | | observed | | Purity |
| Structure | No. | (min) | MW | m/z | Ion | Method |
| 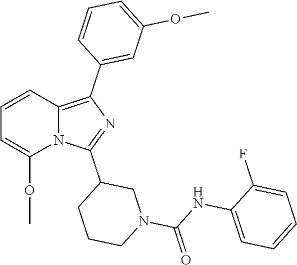 [SMILES: COc1cccc(-c2nc(C3CCCN(C(=O)Nc4ccccc4F)C3)n3c(OC)cccc23)c1] | 13-1 | 0.91 | 474.2 | 475.2 | [M + H]⁺ | 4 |

### Example 14

### Synthesis of Compound 14-1 and Other Representative Compounds

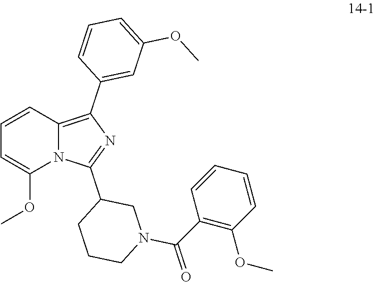

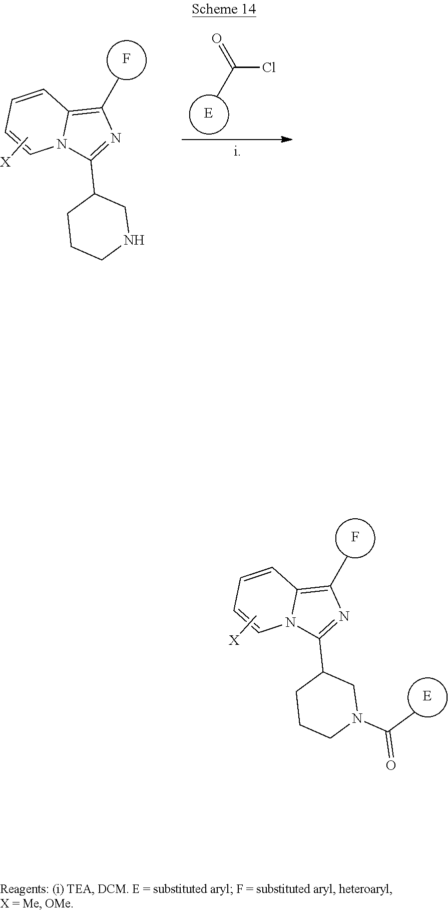

### Step 14-1. Synthesis of (3-(5-methoxy-1-(3-methoxyphenyl)imidazo[1,5-a]pyridin-3-yl)piperidin-1-yl)(2-methoxyphenyl)methanone (Compound 14-1)

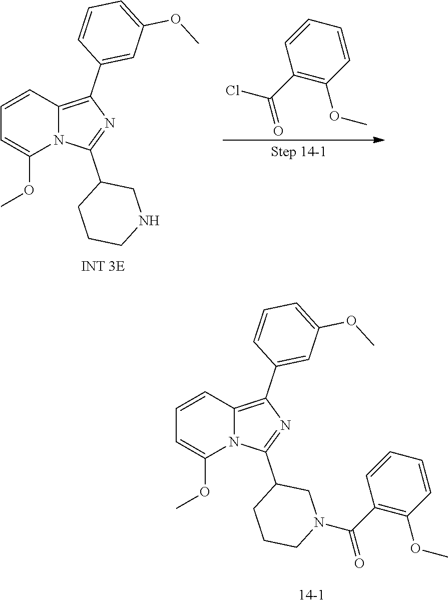 [SMILES: COc1cccc(-c2nc(C3CCCNC3)n3c(OC)cccc23)c1 ; COc1ccccc1C(=O)Cl ; COc1cccc(-c2nc(C3CCCN(C(=O)c4ccccc4OC)C3)n3c(OC)cccc23)c1]

A solution of INT 13E (50 mg, 0.13 mmol) in DCM (1.8 mL) was treated with 2-methoxybenzoyl chloride (19.83 UL, 0.13 mmol) and TEA (70.2 UL, 0.5 mmol). After stirring at 20° C. for 30 min, the reaction mixture was diluted with NaHCO₃ (aq.) and then extracted with EA (3×). The combined organic layers were dried (Na₂SO₄), filtered and concentrated under reduced pressure to give a residue that was purified by reverse-phase HPLC (H₂O (10 mM NH₄HCO₃)/ACN) to afford 18 mg (28%) of (3-(5-methoxy-1-(3-methoxyphenyl) imidazo[1,5-a]pyridin-3-yl) piperidin-1-yl) (2-methoxyphenyl) methanone (Compound 14-1) as a white solid. LCMS-ESI (m/z) calculated for C₂₈H₂₉N₃O₄: 471.6; found 472.2 [M+H]⁺, t_{R}=0.927 min (Method 4).

The compounds list in Table 14 were made using the procedures of Scheme 14:

TABLE 14

| | | Purity | | | | |
|---|---|---|---|---|---|---|
| | Cpd | RT | | Observed | | Purity |
| Structure | No. | (min) | MW | m/z | Ion | Method |
| 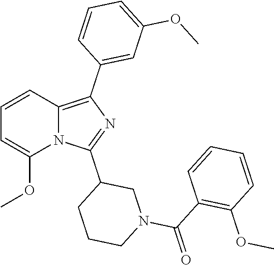 [SMILES: COc1cccc(-c2nc(C3CCCN(C(=O)c4ccccc4OC)C3)n3c(OC)cccc23)c1] | 14-1 | 0.93 | 471.2 | 472.2 | [M + H]⁺ | 4 |
| 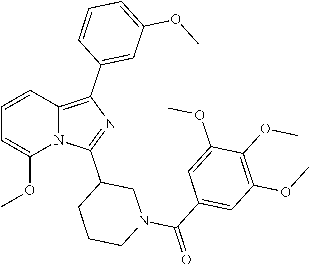 [SMILES: COc1cccc(-c2nc(C3CCCN(C(=O)c4cc(OC)c(OC)c(OC)c4)C3)n3c(OC)cccc23)c1] | 14-2 | 0.931 | 531.2 | 532.3 | [M + H]⁺ | 4 |
| 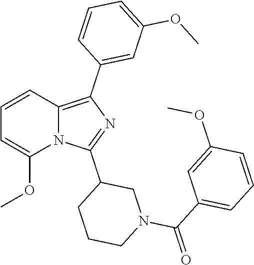 [SMILES: COc1cccc(C(=O)N2CCCC(c3nc(-c4cccc(OC)c4)c4cccc(OC)n34)C2)c1] | 14-3 | 0.863 | 471.2 | 472.3 | [M + H]⁺ | 4 |
| 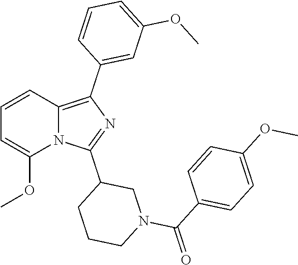 [SMILES: COc1ccc(C(=O)N2CCCC(c3nc(-c4cccc(OC)c4)c4cccc(OC)n34)C2)cc1] | 14-4 | 0.853 | 471.2 | 472.2 | [M + H]⁺ | 4 |
| 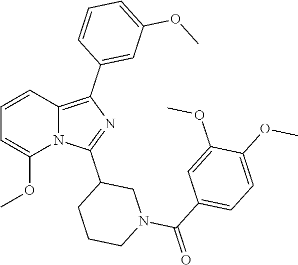 [SMILES: COc1cccc(-c2nc(C3CCCN(C(=O)c4ccc(OC)c(OC)c4)C3)n3c(OC)cccc23)c1] | 14-5 | 0.829 | 501.2 | 502.3 | [M + H]⁺ | 4 |

### Example 15

### Synthesis of Compound 15-1 and Other Representative Compounds

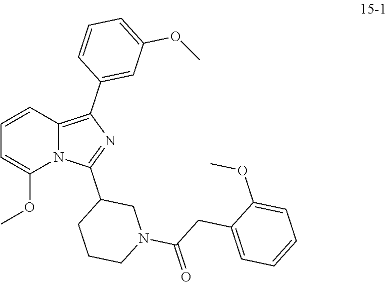 [SMILES: COc1cccc(-c2nc(C3CCCN(C(=O)Cc4ccccc4OC)C3)n3c(OC)cccc23)c1]

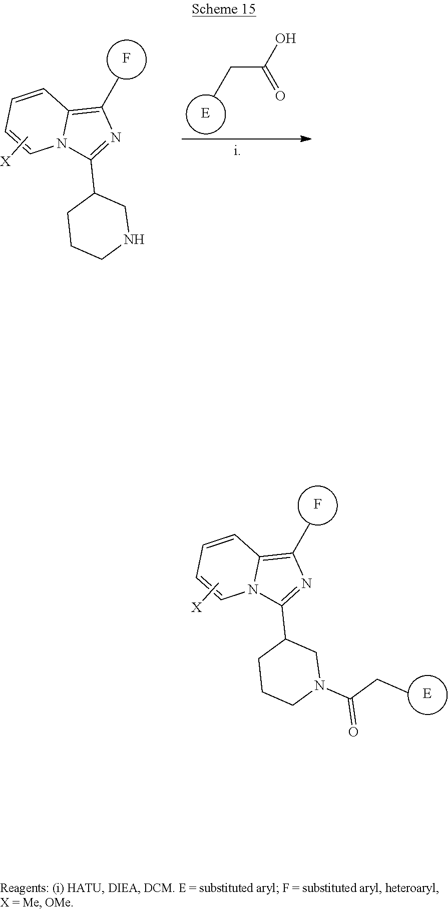

### Step 15-1. Synthesis of 1-(3-(5-methoxy-1-(3-methoxyphenyl)imidazo[1,5-a]pyridin-3-yl)piperidin-1-yl)-2-(2-methoxyphenyl)ethan-1-one (Compound 15-1)

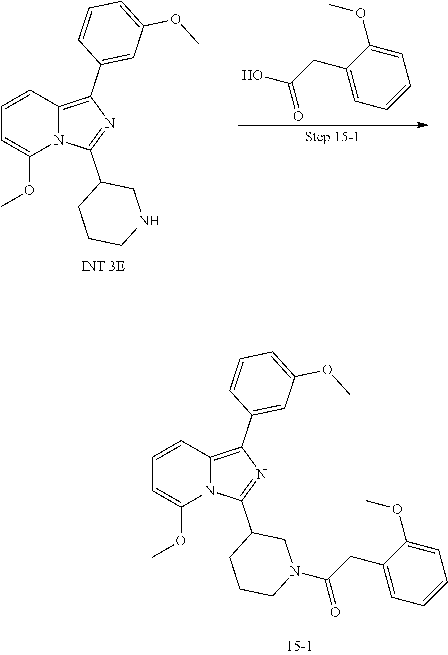 [SMILES: COc1cccc(-c2nc(C3CCCNC3)n3c(OC)cccc23)c1 ; COc1ccccc1CC(=O)O ; COc1cccc(-c2nc(C3CCCN(C(=O)Cc4ccccc4OC)C3)n3c(OC)cccc23)c1]

To a solution of 2-(2-methoxyphenyl)acetic acid (10 mg, 60.1 μmol) in DCM (2.25 mL) were added DIEA (31.5 μL, 180.5 μmol) and HATU (27.46 mg, 72.2 μmol. After 30 min, INT 13E (22.5 mg, 60.2 μmol) was added at 0° C. After stirring at 20° C. for 12 h, the mixture was concentrated to a residue that was purified by prep HPLC (H₂O (10 mM NH₄HCO₃)/ACN) to produce 22.46 mg (38%) of 1-(3-(5-methoxy-1-(3-methoxyphenyl) imidazo[1,5-a]pyridin-3-yl)piperidin-1-yl)-2-(2-methoxyphenyl) ethan-1-one (Compound 15-1) as a white solid. LCMS-ESI (m/z) calculated for C₂₉H₃₁N₃O₄: 485.2; found 486.2 [M+H]⁺, t_{R}=0.75 min (Method 4).

The compounds listed in the Table 15 were made using the procedures of Scheme 15.

TABLE 15

| | | Purity | | | | |
|---|---|---|---|---|---|---|
| | Cpd | RT | | Observed | | Purity |
| Structure | No. | (min) | MW | m/z | Ion | Method |
| 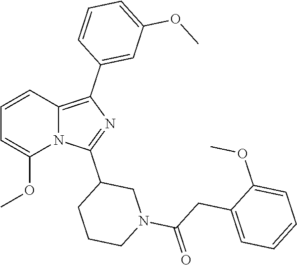 [SMILES: COc1cccc(-c2nc(C3CCCN(C(=O)Cc4ccccc4OC)C3)n3c(OC)cccc23)c1] | 15-1 | 0.948 | 485.2 | 486.3 | [M + H]⁺ | 4 |

### Example 16

### Synthesis of Compound 16-2 and Other Representative Compounds

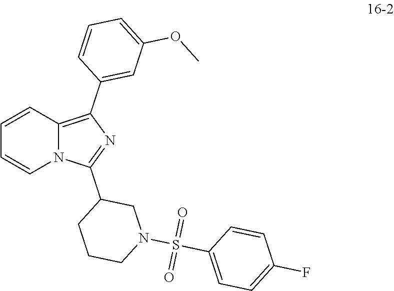 [SMILES: COc1cccc(-c2nc(C3CCCN(S(=O)(=O)c4ccc(F)cc4)C3)n3ccccc23)c1]

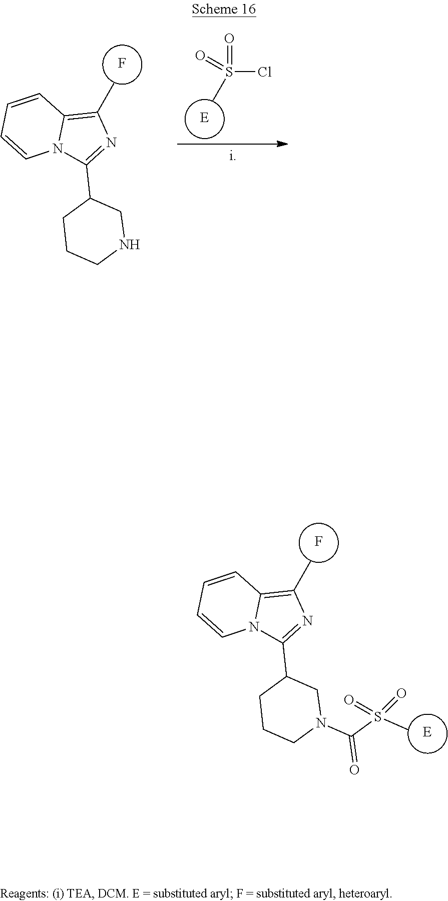

### Step 16-1. Synthesis of 3-(1-((4-fluorophenyl)sulfonyl)piperidin-3-yl)-1-(3-methoxyphenyl)imidazo[1,5-a]pyridine (Compound 16-2)

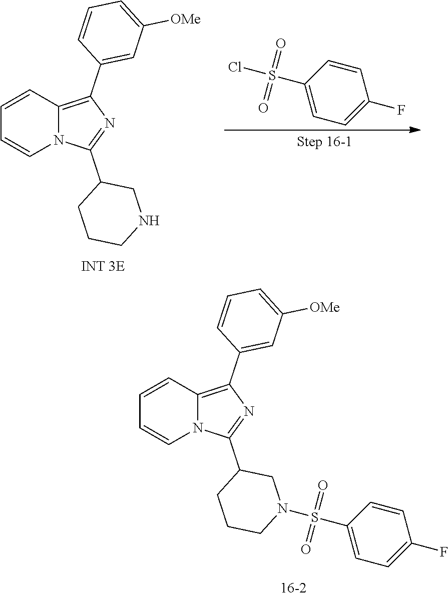 [SMILES: COc1cccc(-c2nc(C3CCCNC3)n3ccccc23)c1 ; O=S(=O)(Cl)c1ccc(F)cc1 ; COc1cccc(-c2nc(C3CCCN(S(=O)(=O)c4ccc(F)cc4)C3)n3ccccc23)c1]

To a solution of INT 3E (60 mg, 0.18 mmol) in DCM (3 mL) were added TEA (73.4 μL, 0.53 mmol) and 3-fluorobenzenesulfonyl chloride (23.4 μL, 175.7 μmol). After stirring at 25° C. for 2 h, the mixture was concentrated under reduced pressure to give a residue that was purified by reverse-phase HPLC chromatography (10 mM NH₄HCO₃/ACN) to afford 24.2 mg (29%) of 3-(1-((4-fluorophenyl)sulfonyl)piperidin-3-yl)-1-(3-methoxyphenyl)imidazo[1,5-a]pyridine (Compound 16-2) as a light yellow solid. LCMS-ESI (m/z) calculated for C₂₅H₂₄FN₃O₃S: 465.2; found 466.2 [M+H]⁺, t_{R}=0.97 min (Method 4). ¹H NMR (400 MHZ, CDCl₃) δ 1.85-2.02 (m, 3H) 2.17 (br s, 1H) 2.39-2.50 (m, 1H) 2.73 (t, J=11.43 Hz, 1H) 3.37 (br s, 1H) 3.88 (s, 3H) 3.97 (br d, J=11.25 Hz, 1H) 4.05-4.13 (m, 1H) 6.61-6.69 (m, 1H) 6.75-6.86 (m, 2H) 7.29-7.42 (m, 4H) 7.46-7.59 (m, 3H) 7.78 (d, J=9.29 Hz, 1H) 7.86 (d, J=7.21 Hz, 1H).

The compounds listed in Table 16 were made using the procedures of Scheme 16.

TABLE 16

| | | Purity | | | | |
|---|---|---|---|---|---|---|
| | Cpd | RT | | observed | | Purity |
| Structure | No. | (min) | MW | m/z | Ion | Method |
| 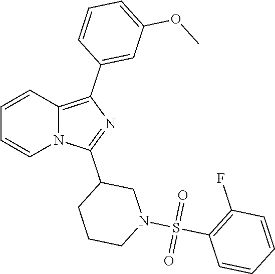 [SMILES: COc1cccc(-c2nc(C3CCCN(S(=O)(=O)c4ccccc4F)C3)n3ccccc23)c1] | 16-1 | 0.96 | 465.2 | 466.2 | [M + H]⁺ | 4 |
| 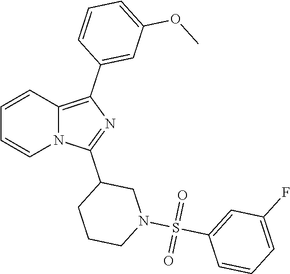 [SMILES: COc1cccc(-c2nc(C3CCCN(S(=O)(=O)c4cccc(F)c4)C3)n3ccccc23)c1] | 16-2 | 0.973 | 465.2 | 466.2 | [M + H]⁺ | 4 |
| 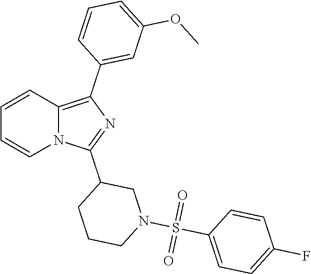 [SMILES: COc1cccc(-c2nc(C3CCCN(S(=O)(=O)c4ccc(F)cc4)C3)n3ccccc23)c1] | 16-3 | 0.968 | 465.2 | 466.2 | [M + H]⁺ | 4 |
| 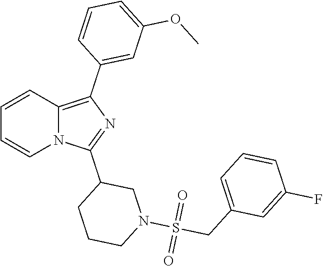 [SMILES: COc1cccc(-c2nc(C3CCCN(S(=O)(=O)Cc4cccc(F)c4)C3)n3ccccc23)c1] | 16-4 | 0.959 | 479.2 | 480.2 | [M + H]⁺ | 4 |
| 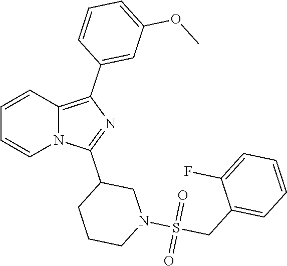 [SMILES: COc1cccc(-c2nc(C3CCCN(S(=O)(=O)Cc4ccccc4F)C3)n3ccccc23)c1] | 16-5 | 0.961 | 479.2 | 480.2 | [M + H]⁺ | 4 |
| 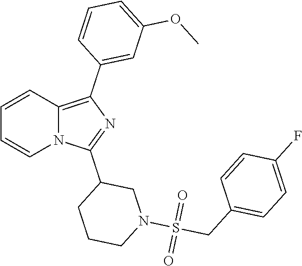 [SMILES: COc1cccc(-c2nc(C3CCCN(S(=O)(=O)Cc4ccc(F)cc4)C3)n3ccccc23)c1] | 16-6 | 0.961 | 479.2 | 480.2 | [M + H]⁺ | 4 |
| 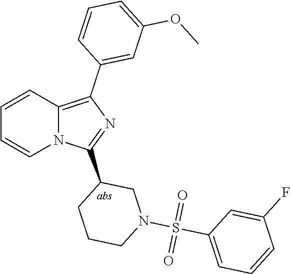 [SMILES: COc1cccc(-c2nc([C@@H]3CCCN(S(=O)(=O)c4cccc(F)c4)C3)n3ccccc23)c1] | 16-7 | 12.29 | 465.2 | 465.8 | [M + H]⁺ | 2 |
| 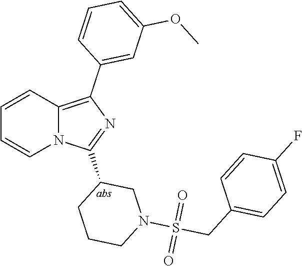 [SMILES: COc1cccc(-c2nc([C@H]3CCCN(S(=O)(=O)Cc4ccc(F)cc4)C3)n3ccccc23)c1] | 16-8 | 12.07 | 479.2 | 479.8 | [M + H]⁺ | 2 |
| 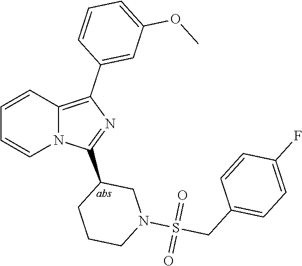 [SMILES: COc1cccc(-c2nc([C@@H]3CCCN(S(=O)(=O)Cc4ccc(F)cc4)C3)n3ccccc23)c1] | 16-9 | 12.21 | 479.2 | 479.8 | [M + H]⁺ | 2 |
| 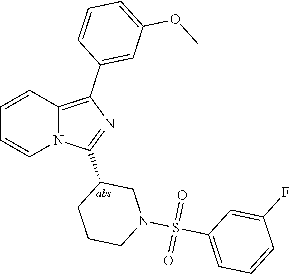 [SMILES: COc1cccc(-c2nc([C@H]3CCCN(S(=O)(=O)c4cccc(F)c4)C3)n3ccccc23)c1] | 16-10 | 12.3 | 465.2 | 465.8 | [M + H]⁺ | 2 |

### Example 17

### Synthesis of Compound 17-1 and Other Representative Compounds

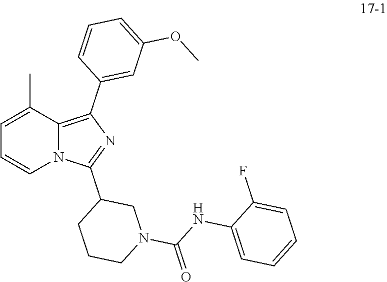 [SMILES: COc1cccc(-c2nc(C3CCCN(C(=O)Nc4ccccc4F)C3)n3cccc(C)c23)c1]

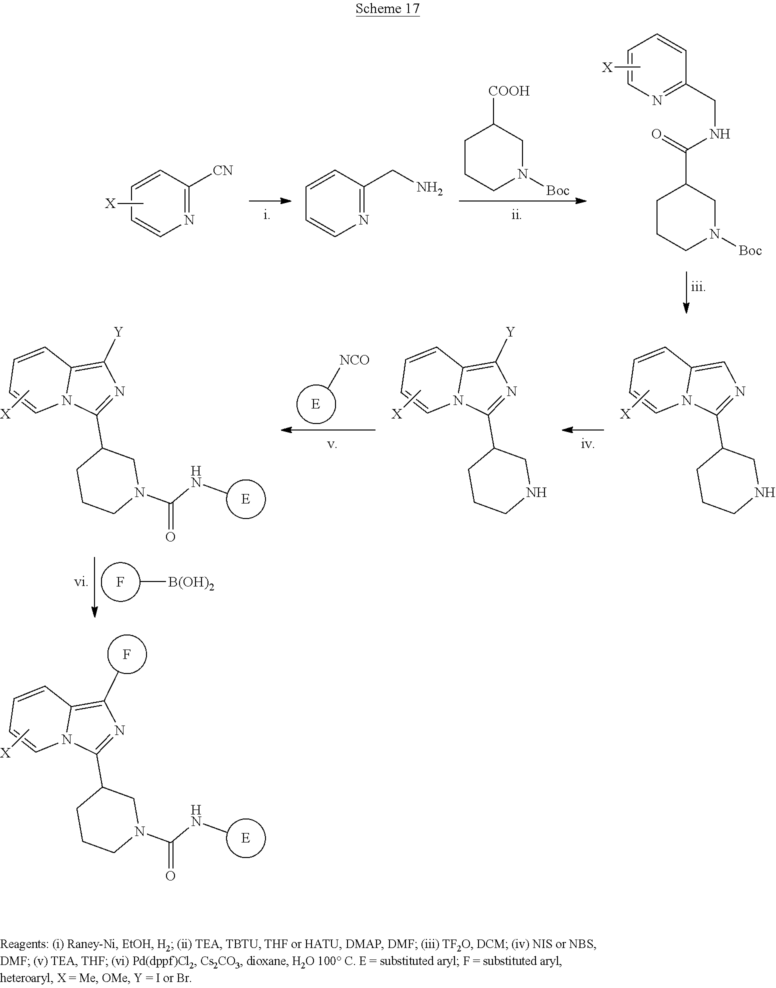

### Step 17-1. Synthesis of (3-methyl-1,2-dihydropyridin-2-yl)methanamine (INT 17A)

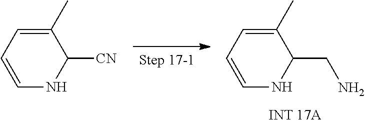

A mixture of 3-methylpyridine-2-carbonitrile (10 g, 84.7 mmol) and Raney-Ni (4.00 g, 4.67 mmol, 10% purity) in EtOH (80 mL) was stirred at 25° C. for 2 h under H₂ atmosphere (50 Psi). The mixture was filtered and concentrated to provide 7 g (51%) of (3-methyl-1,2-dihydropyridin-2-yl)methanamine (INT 17A) as a purple oil that was used in the next step without further purification. ¹H NMR (400 MHZ, DMSO-D₆) δ=8.42 (d, 1H), 7.43 (d, 1H), 7.09 (d, 1H), 3.95 (s, 2H), 2.29 (2, 3H).

### Step 17-2. Synthesis of tert-butyl 3-(((3-methyl-1,2-dihydropyridin-2-yl)methyl)carbamoyl)piperidine-1-carboxylate (INT 17B)

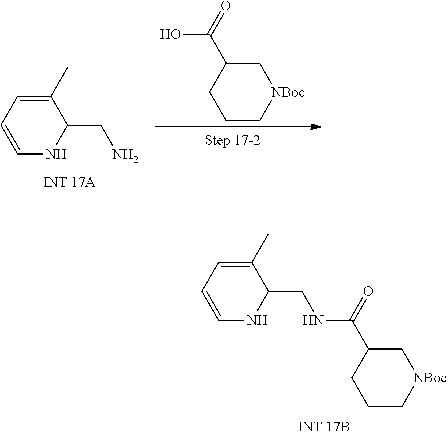

A mixture of 1-tert-butoxycarbonylpiperidine-3-carboxylic acid (10.32 g, 45.02 mmol), TEA (17.09 mL, 122.78 mmol), and TBTU (15.77 g, 49.11 mmol) in THF (80 mL) was stirred at 25° C. for 0.25 h. INT 17A (5 g, 40.93 mmol) was added and the mixture was stirred at 25° C. for 0.75 h. The reaction mixture was poured into H₂O and extracted with EA (3×). The combined organic layer was washed with brine, dried (Na₂SO₄), filtered and concentrated. The resulting residue was purified by SiO₂ chromatography (petroleum ether/EA) to provide 6 g (42%) of tert-butyl 3-(((3-methyl-1,2-dihydropyridin-2-yl) methyl) carbamoyl) piperidine-1-carboxylate (INT 17B) as a brown oil that was used in the next step without further purification. LCMS-ESI (m/z) calculated for C₁₈H₁₉N₃O₃: 335.2; found 334.4 [M−H]⁺, t_{R}=0.647 min (Method 4).

### Step 17-3. Synthesis of 8-methyl-3-(piperidin-3-yl)imidazo[1,5-a]pyridine (INT 17C)

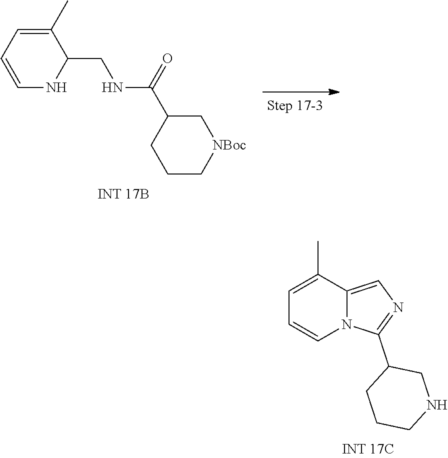 [SMILES: CC1=CC=CNC1CNC(=O)C1CCCN(C(=O)OC(C)(C)C)C1 ; Cc1cccn2c(C3CCCNC3)ncc12]

A mixture of INT 17B (5.0 g, 15.00 mmol), Tf₂O (3.22 mL, 19.49 mmol) and 2-methoxypyridine (1.71 mL, 16.50 mmol) in DCM (60 mL) was stirred at 25° C. for 16 h. The mixture was diluted with petroleum ether and filtered. The filter cake was collected to provide 3.2 g (99%) of 8-methyl-3-(piperidin-3-yl)imidazo[1,5-a]pyridine (INT 17C) as a yellow solid that was used in the next step without further purification. LCMS-ESI (m/z) calculated for C₁₃H₁₇N₃: 215.1; found 216.6 [M+H]⁺, t_{R}=0.617 min (Method 4).

### Step 17-4. Synthesis of 1-bromo-8-methyl-3-(piperidin-3-yl)imidazo[1,5-a]pyridine (INT 17D)

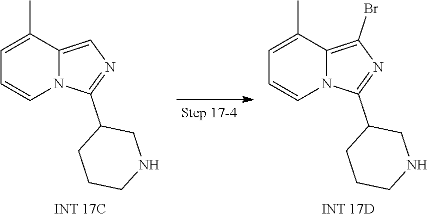 [SMILES: Cc1cccn2c(C3CCCNC3)ncc12 ; Cc1cccn2c(C3CCCNC3)nc(Br)c12]

A mixture of INT 17C (2 g, 9.29 mmol) and NBS (1.82 g, 10.22 mmol) in DMF (50 mL) was stirred at 25° C. for 0.15 h. The mixture was concentrated and purified by reversed phase SiO₂ chromatography to provide 1.0 g (37%) of 1-bromo-8-methyl-3-(piperidin-3-yl)imidazo[1,5-a]pyridine (INT 17D) as a yellow solid. LCMS-ESI (m/z) calculated for C₁₃H₁₆BrN₃: 293.1; found 294.3 [M+H]⁺, t_{R}=0.635 min (Method 4).

### Step 17-5. Synthesis of 3-(1-bromo-8-methylimidazo[1,5-a]pyridin-3-yl)-N-(2-fluorophenyl)piperidine-1-carboxamide (INT 17E)

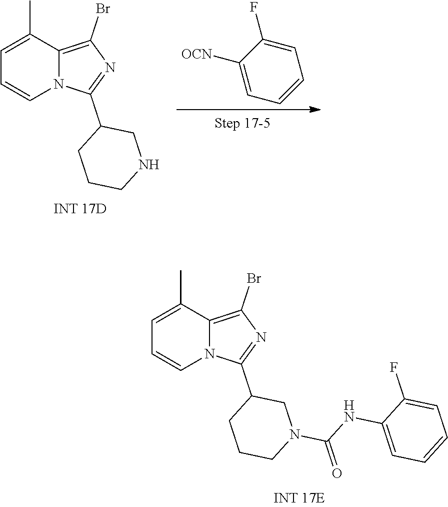

A mixture of INT 17D (600 mg, 2.04 mmol), 1-fluoro-2-isocyanatobenzene (275 μL, 2.45 mmol), and TEA (567.8 uL, 4.08 mmol) in THF (10 mL) was stirred at 25° C. for 0.25 h. The mixture was concentrated and purified by SiO₂ chromatography (Petroleum ether/EA) to provide 600 mg (68%) of 3-(1-bromo-8-methylimidazo[1,5-a]pyridin-3-yl)-N-(2-fluorophenyl)piperidine-1-carboxamide (INT 17E) as a yellow solid. LCMS-ESI (m/z) calculated for C₂₀H₂₀BrFN₄O: 430.1; found 433.3 [M+H]⁺, t_{R}=0.885 min (Method 4).

### Step 17-6. Synthesis of N-(2-fluorophenyl)-3-(1-(3-methoxyphenyl)-8-methylimidazo[1,5-a]pyridin-3-yl)piperidine-1-carboxamide (Compound 17-1)

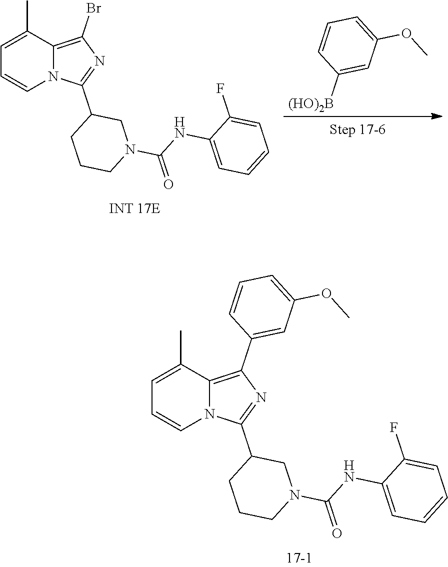 [SMILES: Cc1cccn2c(C3CCCN(C(=O)Nc4ccccc4F)C3)nc(Br)c12 ; COc1cccc(B(O)O)c1 ; COc1cccc(-c2nc(C3CCCN(C(=O)Nc4ccccc4F)C3)n3cccc(C)c23)c1]

A mixture of INT 17E (500 mg, 1.16 mmol), (3-methoxyphenyl) boronic acid (193.78 mg, 1.28 mmol), Pd(dppf)Cl₂ (84.83 mg, 115.93 μmol), and Cs₂CO₃ (755.43 mg, 2.32 mmol) in dioxane (5 mL) and H₂O (1 mL) was stirred at 100° C. for 1 h under N₂. The mixture was filtered, concentrated, and purified by SiO₂ chromatography (Petroleum ether/EA) to provide crude product that was further purified by Prep HPLC chromatography and lyophilized to provide 74 mg (14%) of N-(2-fluorophenyl)-3-(1-(3-methoxyphenyl)-8-methylimidazo[1,5-a]pyridin-3-yl) piperidine-1-carboxamide (Compound 17-1) as a white solid. LCMS-ESI (m/z) calculated for C₂₇H₂₇FN₄O₂: 458.2; found 459.1 [M+H]⁺, t_{R}=0.761 min (Method 4).

The compounds listed in Table 17 were made using the procedures of Scheme 17.

TABLE 17

| | | Purity | | | | |
|---|---|---|---|---|---|---|
| | Cpd | RT | | observed | | Purity |
| Structure | No. | (min) | MW | m/z | Ion | Method |
| 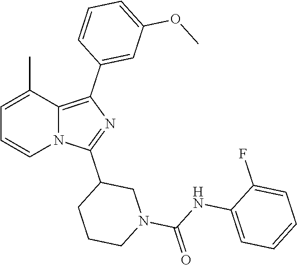 [SMILES: COc1cccc(-c2nc(C3CCCN(C(=O)Nc4ccccc4F)C3)n3cccc(C)c23)c1] | 17-1 | 0.761 | 458.2 | 459.1 | [M + H]⁺ | 4 |
| 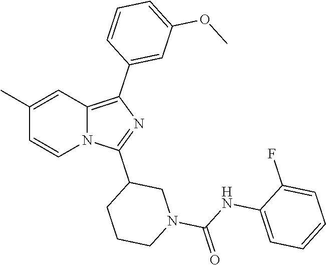 [SMILES: COc1cccc(-c2nc(C3CCCN(C(=O)Nc4ccccc4F)C3)n3ccc(C)cc23)c1] | 17-2 | 0.811 | 458.2 | 459.3 | [M + H]⁺ | 4 |
| 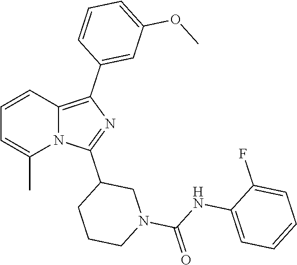 [SMILES: COc1cccc(-c2nc(C3CCCN(C(=O)Nc4ccccc4F)C3)n3c(C)cccc23)c1] | 17-3 | 0.82 | 458.2 | 459.3 | [M + H]⁺ | 4 |

### Example 18

### Synthesis of Compound 18-1 and Other Representative Compounds

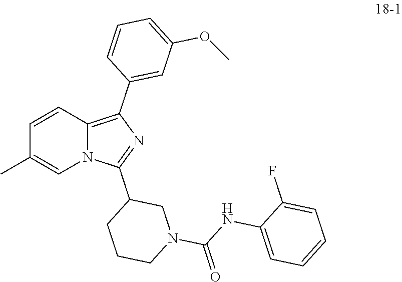 [SMILES: COc1cccc(-c2nc(C3CCCN(C(=O)Nc4ccccc4F)C3)n3cc(C)ccc23)c1]

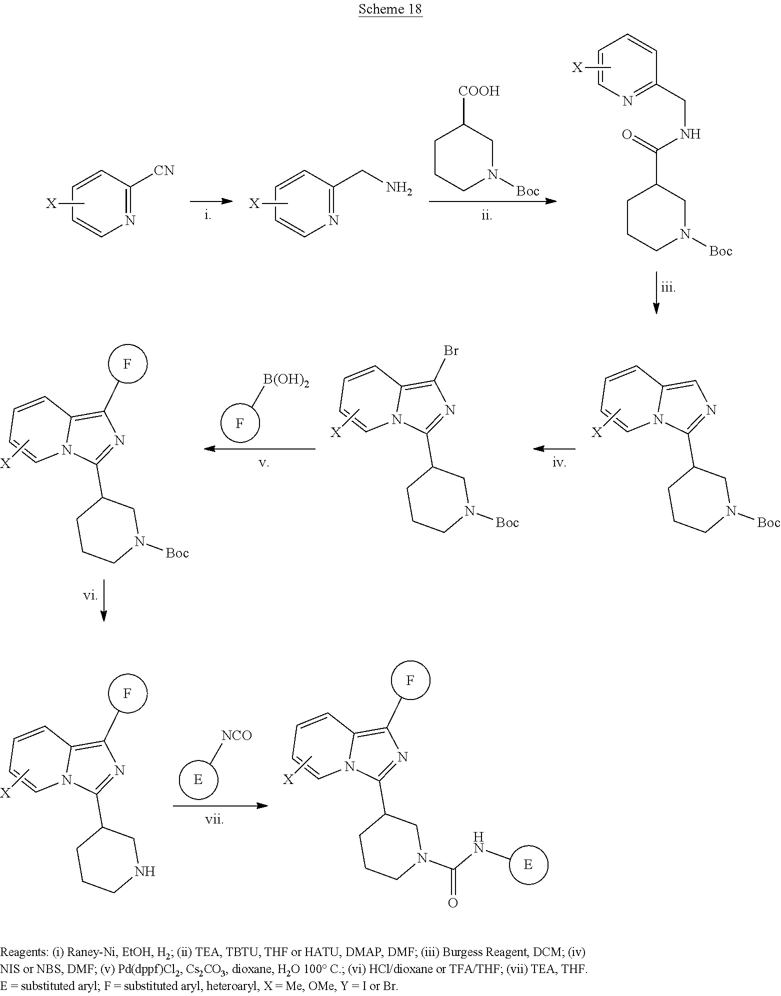

### Step 18-1. Synthesis of (5-methyl-1,2-dihydropyridin-2-yl)methanamine (INT 18A)

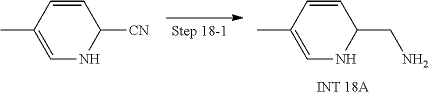

A mixture of 5-methylpyridine-2-carbonitrile (1 g, 8.46 mmol) and Raney-Ni (0.4 g, 4.7 mmol) in EtOH (10 mL) was stirred at 25° C. for 2 h under H₂ atmosphere. The mixture was filtered and concentrated to provide 7 g (51%) of (5-methyl-1,2-dihydropyridin-2-yl)methanamine (INT 18A) as a yellow oil that was used in the next step without further purification. ¹H NMR (400 MHz, DMSO-D₆) δ=8.31 (d, 1H), 7.54 (d, 1H), 7.325 (d, 1H), 3.75 (s, 2H), 2.25 (2, 3H).

### Step 18-2. Synthesis of tert-butyl 3-(((5-methyl-1,2-dihydropyridin-2-yl)methyl)carbamoyl)piperidine-1-carboxylate (INT 18B)

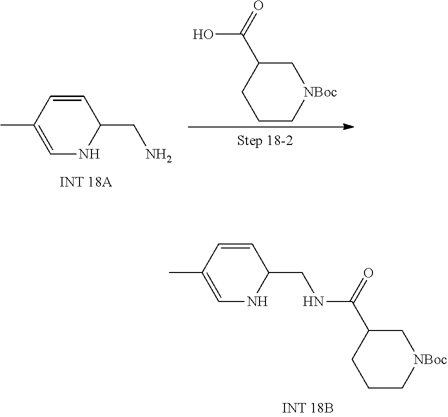

To a mixture of 1-tert-butoxycarbonylpiperidine-3-carboxylic acid (1.41 g, 6.1 mmol), HATU (2.33 g, 6.1 mmol) and DMAP (750.0 mg, 6.1 mmol) in DMF (5 mL) was added (5-methyl-2-pyridyl)methanamine INT 18A (500 mg, 4.1 mmol). The mixture was stirred at 25° C. for 2 h then diluted with H₂O (50 mL) and extracted with EA (3×). The combined organic layers were washed with H₂O and brine, then dried (Na₂SO₄), filtered and concentrated. The resulting residue was purified by SiO₂ chromatography (DCM/MeOH) to provide 1.6 g (60%) of tert-butyl 3-(((5-methyl-1,2-dihydropyridin-2-yl) methyl) carbamoyl) piperidine-1-carboxylate (INT 18B) as a black oil that was used in the next step without further purification. LCMS-ESI (m/z) calculated for C₁₈H₁₉N₃O₃: 335.2; found 334.4 [M−H]⁺, t_{R}=0.78 min (Method 8).

### Step 17-3. Synthesis of tert-butyl 3-(6-methylimidazo[1,5-a]pyridin-3-yl)piperidine-1-carboxylate (INT 18C)

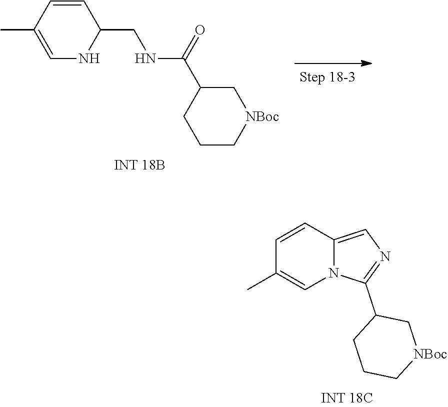 [SMILES: CC1=CNC(CNC(=O)C2CCCN(C(=O)OC(C)(C)C)C2)C=C1 ; Cc1ccc2cnc(C3CCCN(C(=O)OC(C)(C)C)C3)n2c1]

A mixture of INT 18B (1.5 g, 4.5 mmol) and Burgess Reagent (857.7 mg, 3.6 mmol) in DCM (10 mL) was stirred at 25° C. for 16 h under an atmosphere of N₂. The mixture was diluted with H₂O and extracted with EA (3×). The combined organic layers were washed with H₂O and brine, then dried (Na₂SO₄), filtered, and concentrated to provide 1.35 g (89%) of tert-butyl 3-(6-methylimidazo[1,5-a]pyridin-3-yl)piperidine-1-carboxylate (INT 18C) as a yellow oil that was used in the next step without further purification. LCMS-ESI (m/z) calculated for C₁₈H₂₅N₃O₂: 315.1; found 316.26 [M+H]⁺, t_{R}=0.717 min (Method 4).

### Step 18-4. Synthesis of tert-butyl 3-(1-bromo-6-methylimidazo[1,5-a]pyridin-3-yl)piperidine-1-carboxylate (INT 18D)

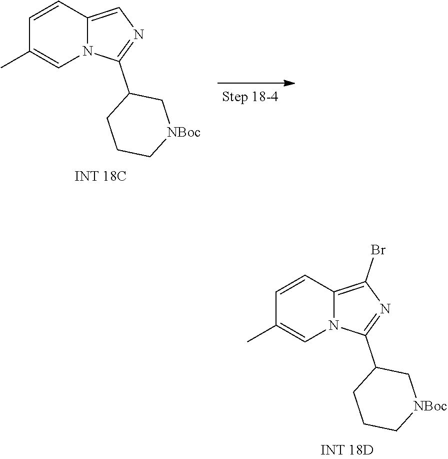 [SMILES: Cc1ccc2cnc(C3CCCN(C(=O)OC(C)(C)C)C3)n2c1 ; Cc1ccc2c(Br)nc(C3CCCN(C(=O)OC(C)(C)C)C3)n2c1]

To a mixture of INT 18C (1.35 g, 4.27 mmol) in DCM (10 mL) was added NBS (1.14 g, 6.41 mmol). After stirring at 25° C. for 1 h under N₂, the mixture was concentrated and purified by reversed phase SiO₂ chromatography (MeOH/DCM) to provide 35 mg (2%) of tert-butyl 3-(1-bromo-6-methylimidazo[1,5-a]pyridin-3-yl)piperidine-1-carboxylate (INT 18D) as a yellow oil. LCMS-ESI (m/z) calculated for C₁₈H₂₄BrN₃O₂: 393.1; found 394.1 [M+H]⁺, t_{R}=2.68 min (Method 5).

### Step 18-5. Synthesis of tert-butyl 3-(1-(3-methoxyphenyl)-6-methylimidazo[1,5-a]pyridin-3-yl)piperidine-1-carboxylate (INT 18E)

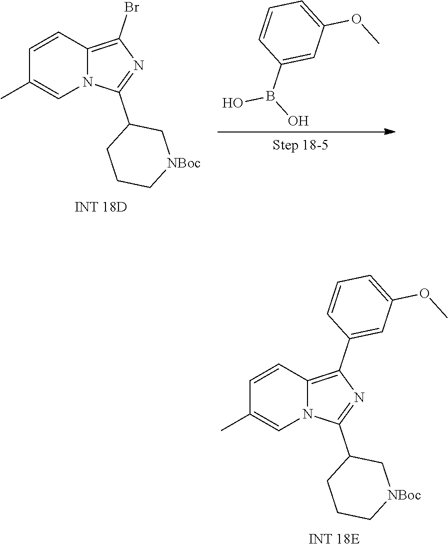

To a solution INT 18D (35 mg, 88.8 μmol) and (3-methoxyphenyl)boronic acid (20.23 mg, 133.2 μmol) in H₂O (0.25 mL) and 1,4-dioxane (1 mL) were added Cs₂CO₃ (57.84 mg, 177.5 μmol) and Pd(dppf)Cl₂ (6.49 mg, 8.88 μmol) at 25° C. After stirring at 100° C. for 1 h under N₂, the mixture was concentrated and purified by prep TLC (1:1 Petroleum ether: EA) to provide 50 mg (75%) of tert-butyl 3-(1-(3-methoxyphenyl)-6-methylimidazo[1,5-a]pyridin-3-yl)piperidine-1-carboxylate (INT 18E) as a yellow oil. LCMS-ESI (m/z) calculated for C₂₅H₃₁N₃O₃: 421.5; found 422.2 [M+H]⁺, t_{R}=1.29 min (Method 9).

### Step 18-6. Synthesis of 1-(3-methoxyphenyl)-6-methyl-3-(piperidin-3-yl)imidazo[1,5-a]pyridine (INT 18F)

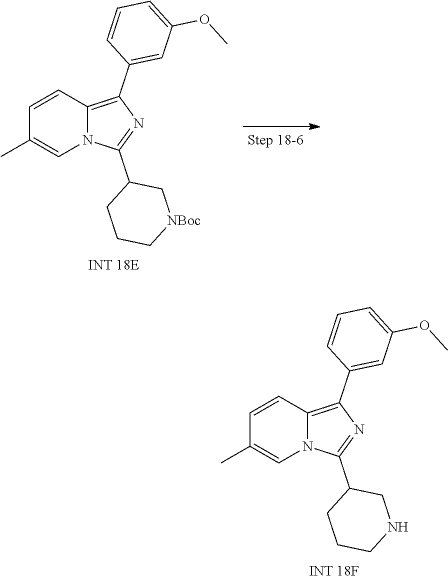

A solution of INT 18E (20 mg, 47.45 μmol) in 1M HCl/Dioxane (1 mL) was stirred at 25° C. for 1 h under N₂. The mixture was concentrated to provide 35 mg of crude 1-(3-methoxyphenyl)-6-methyl-3-(piperidin-3-yl)imidazo[1,5-a]pyridine (INT 18F) as a yellow solid that was used in the next step without further purification. LCMS-ESI (m/z) calculated for C₂₀H₂₃N₃O: 321.4; found 322.2 [M+H]⁺, t_{R}=0.76 min (Method 4).

### Step 18-7. Synthesis of N-(2-fluorophenyl)-3-(1-(3-methoxyphenyl)-6-methylimidazo[1,5-a]pyridin-3-yl)piperidine-1-carboxamide (Compound 18-1)

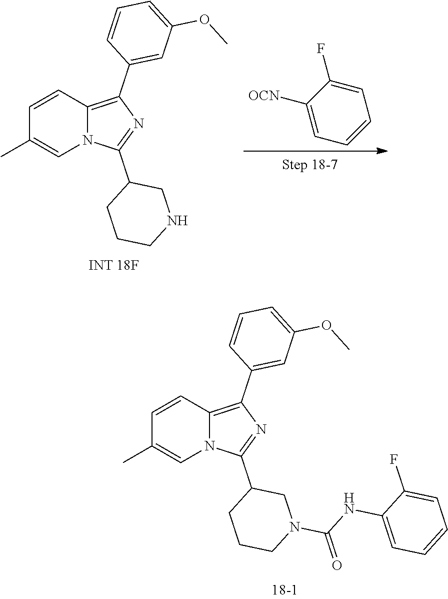

To a solution of INT 18F (35 mg, 108.89 μmol) in THF (1 mL) were added TEA (30.3 μL, 217.8 μmol) and 1-fluoro-2-isocyanato-benzene (14.69 μL, 130.67 μmol) at 25° C. The resulting mixture was stirred at 25° C. for 2 hr, then was concentrated and purified by prep HPLC (water (0.05% HCl)/ACN). The desired fractions were combined and lyophilized to provide 3 mg (6%) of N-(2-fluorophenyl)-3-(1-(3-methoxyphenyl)-6-methylimidazo[1,5-a]pyridin-3-yl)piperidine-1-carboxamide (Compound 18-1) as a white solid. LCMS-ESI (m/z) calculated for C27H₂₇FN₄O₂: 458.2; found 459.3 [M+H]⁺, t_{R}=1.927 min (Method 8). ¹H NMR (400 MHZ, CD₃OD) δ=8.39 (s, 1H), 7.93 (d, J=9.6 Hz, 1H), 7.58-7.44 (m, 2H), 7.40-7.31 (m, 2H), 7.23-7.11 (m, 5H), 4.36 (dd, J=3.4, 13.4 Hz, 1H), 4.10 (br d, J=13.8 Hz, 1H), 3.88 (s, 3H), 3.88-3.81 (m, 1H), 3.61 (dd, J=8.8, 13.6 Hz, 1H), 3.44 (td, J=6.8, 13.6 Hz, 1H), 2.44 (s, 3H), 2.41-2.34 (m, 1H), 2.34-2.22 (m, 1H), 1.94-1.82 (m, 2H).

The compounds listed in Table 18 were made using the procedures of Scheme 18.

TABLE 18

| | | Purity | | | | |
|---|---|---|---|---|---|---|
| | Cpd | RT | | observed | | Purity |
| Structure | No. | (min) | MW | m/z | Ion | Method |
| 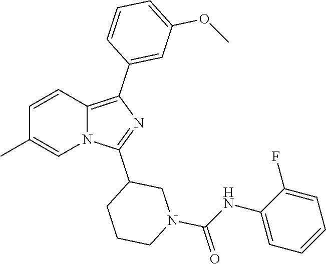 [SMILES: COc1cccc(-c2nc(C3CCCN(C(=O)Nc4ccccc4F)C3)n3cc(C)ccc23)c1] | 18-1 | 1.926 | 458.2 | 459.3 | [M + H]⁺ | 8 |

### Example 19

### Synthesis of Compound 19-3 and Other Representative Compounds

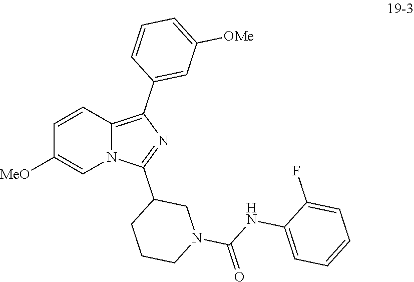 [SMILES: COc1cccc(-c2nc(C3CCCN(C(=O)Nc4ccccc4F)C3)n3cc(OC)ccc23)c1]

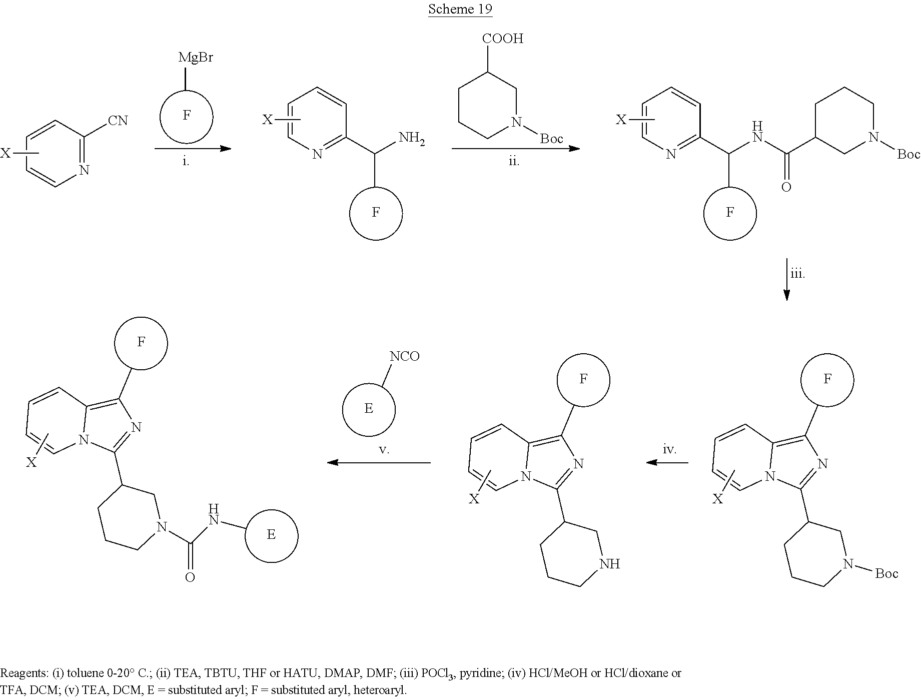

### Step 19-1. Synthesis of (3-methoxyphenyl)(5-methoxypyridin-2-yl)methanamine (INT 19A)

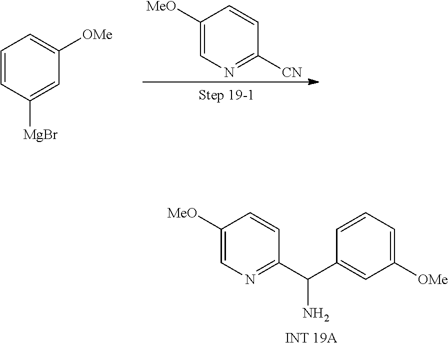

To a solution of 5-methoxypyridine-2-carbonitrile (700 mg, 5.22 mmol) in toluene (15 mL) was added bromo-(3-methoxyphenyl)magnesium (1 M, 5.74 mL) dropwise under nitrogen at 0° C. After the mixture was stirred at 20° C. for 2 h, it was diluted with H₂O, concentrated to remove volatile solvents, then extracted with EA (3×). The combined organic layers were dried (Na₂SO₄), filtered and concentrated to provide 1.5 g (70%) of crude (3-methoxyphenyl)(5-methoxypyridin-2-yl)methanamine (INT 19A) that was used in the next step without further purification. TLC (3:1 petroleum ether:EA) Rr: 0.05 (UV254).

### Step 19-2. Synthesis of tert-butyl 3-(((3-methoxyphenyl)(5-methoxypyridin-2-yl)methyl)carbamoyl)piperidine-1-carboxylate (INT 19B)

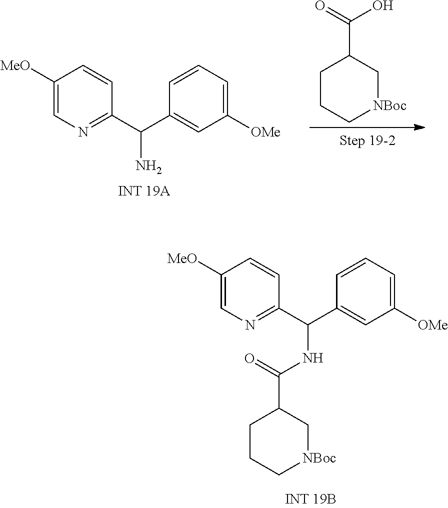 [SMILES: COc1ccc(C(N)c2cccc(OC)c2)nc1 ; CC(C)(C)OC(=O)N1CCCC(C(=O)O)C1 ; COc1ccc(C(NC(=O)C2CCCN(C(=O)OC(C)(C)C)C2)c2cccc(OC)c2)nc1]

To a solution of INT 19A (1.69 g, 7.37 mmol) in DMF (30 mL) were added DIEA (2.14 mL, 12.28 mmol), HATU (2.80 g, 7.37 mmol) and (3-methoxyphenyl)-(5-methoxy-2-pyridyl)methanamine (1.5 g, 6.14 mmol, 1 eq). After the solution was stirred at 20° C. for 2 h, it was diluted with H₂O and extracted with EA (3×). The combined organic layers were dried (Na₂SO₄), filtered and concentrated to provide a residue that was purified by SiO₂ chromatography (EA/pet ether) to produce 1.5 g (48%) of tert-butyl 3-(((3-methoxyphenyl)(5-methoxypyridin-2-yl)methyl)carbamoyl) piperidine-1-carboxylate (INT 19B) as a white solid. LCMS-ESI (m/z) calculated for C₂₅H₃₃N₃O₅: 455.2; found 456.3 [M+H]⁺, t_{R}=0.755 min (Method 5). ¹H NMR (400 MHz, CD₃OD) δ=8.20 (t, J=2.3 Hz, 1H), 7.40-7.34 (m, 1H), 7.32-7.26 (m, 1H), 7.25-7.18 (m, 1H), 6.88-6.77 (m, 3H), 6.10 (s, 1H), 4.10-4.04 (m, 1H), 3.97 (br d, J=13.5 Hz, 1H), 3.86 (d, J=1.0 Hz, 3H), 3.76 (d, J=2.9 Hz, 3H), 3.01-2.85 (m, 1H), 2.81 (br d, J=7.0 Hz, 1H), 2.50 (tt, J=3.7, 10.9 Hz, 1H), 1.96 (br d, J=12.0 Hz, 1H), 1.74-1.64 (m, 2H), 1.52-1.48 (m, 1H), 1.45 (s, 9H).

### Step 19-3. Synthesis of tert-butyl 3-(6-methoxy-1-(3-methoxyphenyl)imidazo[1,5-a]pyridin-3-yl)piperidine-1-carboxylate (INT 19C)

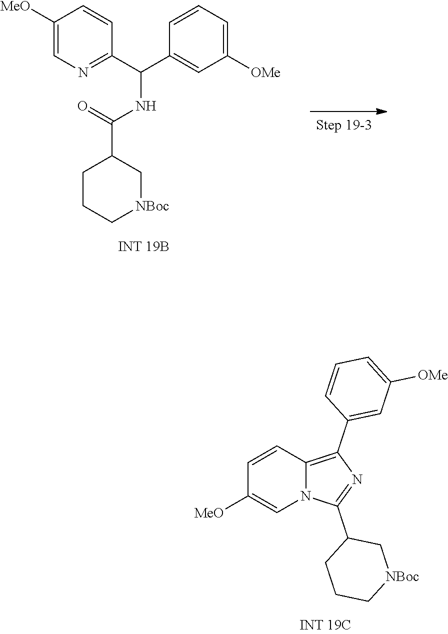 [SMILES: COc1ccc(C(NC(=O)C2CCCN(C(=O)OC(C)(C)C)C2)c2cccc(OC)c2)nc1 ; COc1cccc(-c2nc(C3CCCN(C(=O)OC(C)(C)C)C3)n3cc(OC)ccc23)c1]

To a solution of INT 19B (700 mg, 1.54 mmol) in pyridine (14 mL) was added POCl₃ (285.59 μL, 3.07 mmol) at 0° C. The reaction mixture was diluted with H₂O and extracted with EA (3×). The combined organics were dried (Na₂SO₄), filtered and concentrated under reduced pressure to give 700 mg (83%) of crude tert-butyl 3-(6-methoxy-1-(3-methoxyphenyl)imidazo[1,5-a]pyridin-3-yl)piperidine-1-carboxylate (INT 19C) as a brown solid that was used without further purification. TLC (2:1 EA/petroleum ether), Rr-0.65 (UV254). 1H NMR (400 MHZ, CD₃OD) δ=7.52-7.43 (m, 2H), 7.39-7.28 (m, 3H), 6.92-6.83 (m, 1H), 6.70 (dd, J=2.0, 9.9 Hz, 1H), 4.38-4.03 (m, 2H), 3.91-3.82 (m, 6H), 3.44-3.35 (m, 1H), 3.01 (br s, 2H), 2.19 (br d, J=10.9 Hz, 1H), 2.10-2.00 (m, 1H), 1.91 (br s, 1H), 1.79-1.65 (m, 1H), 1.45 (br d, J=5.5 Hz, 9H).

### Step 19-4. Synthesis of 6-methoxy-1-(3-methoxyphenyl)-3-(piperidin-3-yl)imidazo[1,5-a]pyridine (INT 19D)

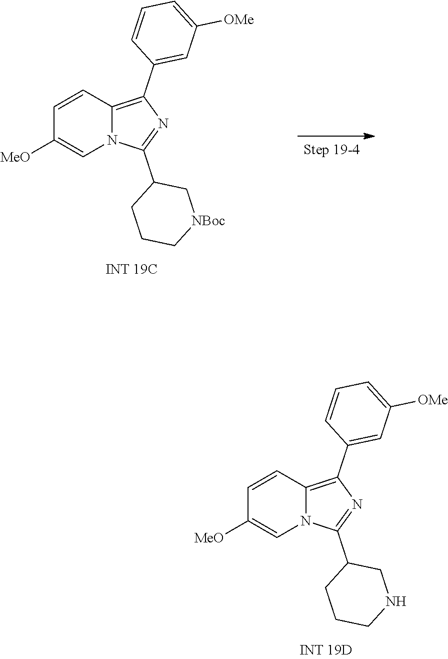 [SMILES: COc1cccc(-c2nc(C3CCCN(C(=O)OC(C)(C)C)C3)n3cc(OC)ccc23)c1 ; COc1cccc(-c2nc(C3CCCNC3)n3cc(OC)ccc23)c1]

A solution of INT 19C (300 mg, 686 μmol) in HCl/MeOH (4 M, 6.00 mL) was stirred at 20° C. for 1 h then concentrated to give 220 mg (86%) of crude 6-methoxy-1-(3-methoxyphenyl)-3-(piperidin-3-yl)imidazo[1,5-a]pyridine (INT 19D) as a yellow solid that was used without further purification. TLC (2:1 EA/petroleum ether), R_{f}=0.00 (UV254). ¹H NMR (400 MHZ, CD₃OD) δ=8.12 (s, 1H), 7.89 (d, J=10.1 Hz, 1H), 7.53 (t, J=8.3 Hz, 1H), 7.38-7.31 (m, 2H), 7.12 (dt, J=1.9, 10.3 Hz, 2H), 4.42 (tt, J=3.7, 11.9 Hz, 1H), 4.04 (s, 3H), 3.91 (s, 3H), 3.78 (br d, J=14.0 Hz, 1H), 3.60 (br d, J=12.9 Hz, 1H), 3.42 (t, J=12.4 Hz, 1H), 3.22-3.11 (m, 1H), 2.39-2.14 (m, 4H).

### Step 19-5. Synthesis of N-(2-fluorophenyl)-3-(6-methoxy-1-(3-methoxyphenyl)imidazo[1,5-a]pyridin-3-yl)piperidine-1-carboxamide (Compound 19-3)

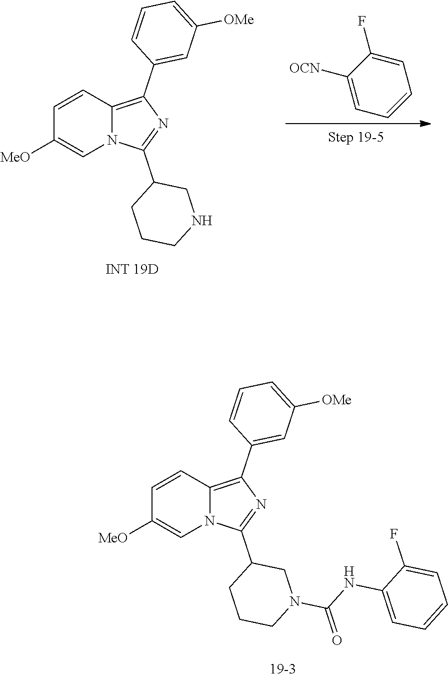

To a solution of INT 19D (220 mg, 652.0 μmol) in DCM (6 mL) were added TEA (272.26 μL, 1.96 mmol) and 1-fluoro-2-isocyanato-benzene (73.28 μL, 652.0 μmol). After the solution was stirred at 20° C. for 2 h, the mixture was diluted with H₂O and extracted with EA (3×). The combined organic layers were dried (Na₂SO₄), concentrated, and purified by prep HPLC (10 mM aqueous NH₄CO₃/CH₃CN) to produce 26.5 mg (8%) of N-(2-fluorophenyl)-3-(6-methoxy-1-(3-methoxyphenyl) imidazo[1,5-a]pyridin-3-yl)piperidine-1-carboxamide (Compound 19-3) as a yellow solid. LCMS-ESI (m/z) calculated for C₂₇H₂₇FN₄O₃: 474.2; found 475.2 [M+H]⁺, t_{R}=0.882 min (Method 4). ¹H NMR (400 MHZ, CDCl₃) δ=7.73-7.67 (m, 2H), 7.51-7.44 (m, 1H), 7.38-7.28 (m, 3H), 7.17-7.08 (m, 3H), 6.86 (td, J=1.9, 7.7 Hz, 1H), 6.70-6.63 (m, 1H), 4.42 (br d, J=13.5 Hz, 1H), 4.21 (br d, J=12.9 Hz, 1H), 3.85 (d, J=5.5 Hz, 6H), 3.44 (tdd, J=3.7, 7.4, 14.8 Hz, 1H), 3.28-3.14 (m, 2H), 2.30-2.11 (m, 2H), 1.98-1.77 (m, 2H).

The compounds listed in Table 19 were made using the procedures of Scheme 19.

TABLE 19

| | | Purity | | | | |
|---|---|---|---|---|---|---|
| | Cpd | RT | | observed | | Purity |
| Structure | No. | (min) | MW | m/z | Ion | Method |
| 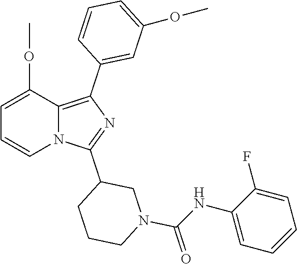 [SMILES: COc1cccc(-c2nc(C3CCCN(C(=O)Nc4ccccc4F)C3)n3cccc(OC)c23)c1] | 19-1 | 0.872 | 474.2 | 475.2 | [M + H]⁺ | 4 |
| 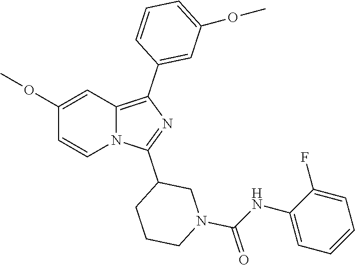 [SMILES: COc1cccc(-c2nc(C3CCCN(C(=O)Nc4ccccc4F)C3)n3ccc(OC)cc23)c1] | 19-2 | 0.88 | 474.2 | 475.2 | [M + H]⁺ | 4 |
| 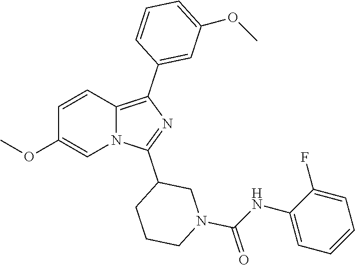 [SMILES: COc1cccc(-c2nc(C3CCCN(C(=O)Nc4ccccc4F)C3)n3cc(OC)ccc23)c1] | 19-3 | 0.882 | 474.2 | 475.2 | [M + H]⁺ | 4 |

### Example 20

### Synthesis of Compound 20-1 and Other Representative Compounds

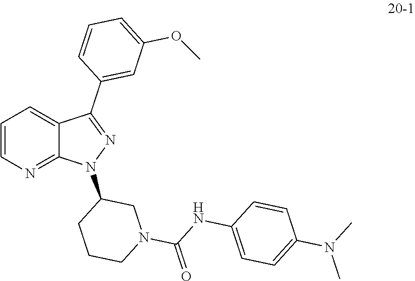 [SMILES: COc1cccc(-c2nn([C@@H]3CCCN(C(=O)Nc4ccc(N(C)C)cc4)C3)c3ncccc23)c1]

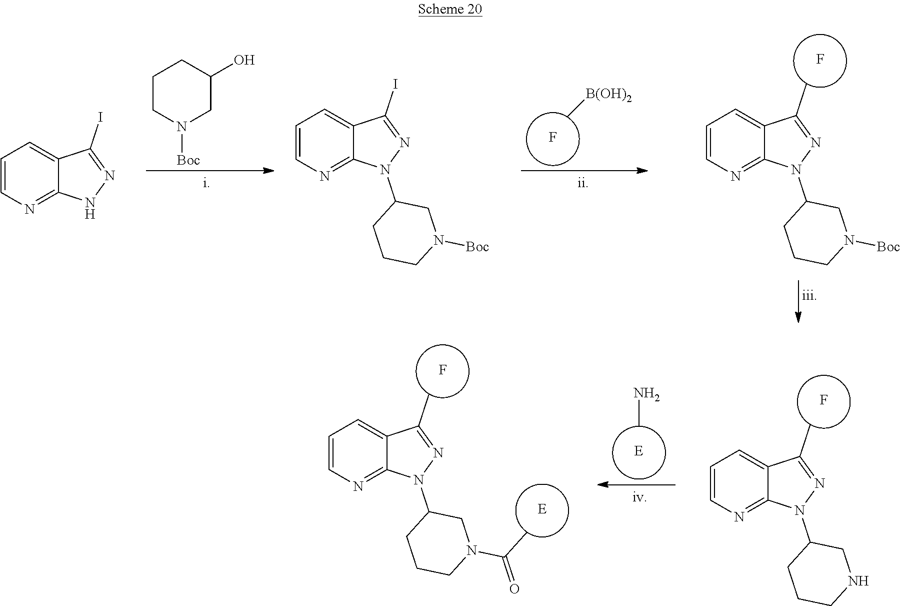

Reagents: (i) PPh₃, DEAD, THF; (ii)) Pd(dppf)Cl₂, Cs₂CO₃, dioxane, H₂O, 90° C.; (iii) HCl/MeOH or HCl/dioxane or TFA, DCM; (iv) DIEA, triphosgene, ACN, 0° C. E=substituted aryl; F=substituted aryl, heteroaryl.

### Step 20-1. Synthesis of tert-butyl (R)-3-(3-iodo-1H-pyrazolo[3,4-b]pyridin-1-yl)piperidine-1-carboxylate (INT 20A)

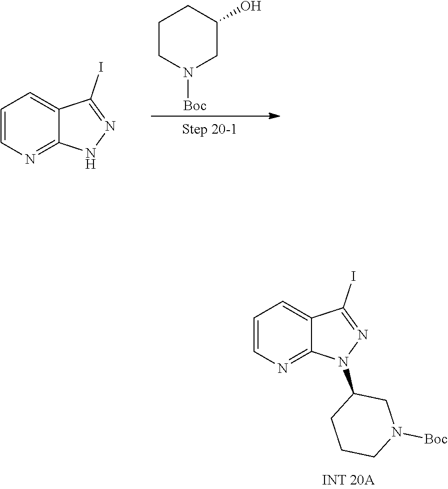 [SMILES: Ic1n[nH]c2ncccc12 ; CC(C)(C)OC(=O)N1CCC[C@H](O)C1 ; CC(C)(C)OC(=O)N1CCC[C@@H](n2nc(I)c3cccnc32)C1]

A 20 mL pressure vial containing a stirring solution of 3-iodo-1H-pyrazolo[3,4-b]pyridine (500 mg, 2.04 mmol) and (S)-1-Boc-3-hydroxypiperidine (411 mg, 2.04 mmol) in THF (8 mL) was charged with triphenylphosphine (803 mg, 3.06 mmol) and diisopropyl azodicarboxylate (595 μL, 3.06 mmol). After stirring for 2 hours at RT, the reaction mixture was poured into H₂O and extracted with EA (3×). The organic layers were combined, washed with brine, and concentrated under reduced pressure. The residue was dissolved in DCM and purified by SiO₂ chromatography (EA/hexanes) to afford 450 mg (51%) of tert-butyl (R)-3-(3-iodo-1H-pyrazolo[3,4-b]pyridin-1-yl)piperidine-1-carboxylate (INT 20A) as a white foam. LCMS-ESI (m/z) calculated for C₁₆H₂₁IN₄O₂: 322.2; found 323.7 [M+H]⁺, t_{R}=1.18 min (Method 11).

### Step 20-2. Synthesis of tert-butyl (R)-3-(3-(3-methoxyphenyl)-1H-pyrazolo[3,4-b]pyridin-1-yl)piperidine-1-carboxylate (INT 20B)

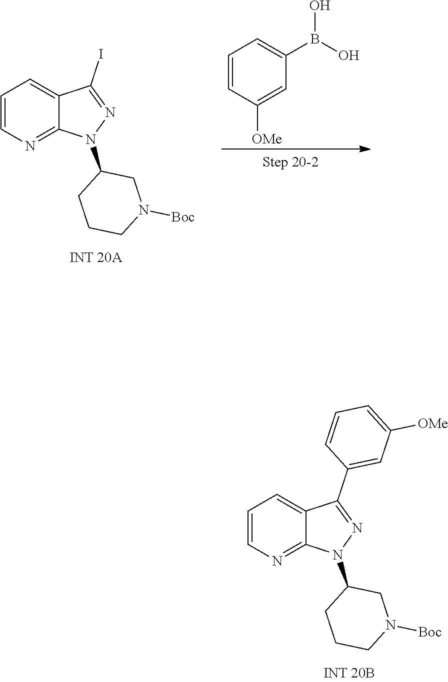 [SMILES: CC(C)(C)OC(=O)N1CCC[C@@H](n2nc(I)c3cccnc32)C1 ; COc1cccc(B(O)O)c1 ; COc1cccc(-c2nn([C@@H]3CCCN(C(=O)OC(C)(C)C)C3)c3ncccc23)c1]

A 20 mL pressure vial containing a mixture of INT 20A (445 mg, 1.04 mmol), 3-methoxyphenylboronic acid (174 mg, 1.14 mmol), [1,1′-Bis (diphenylphosphino) ferrocene] dichloropalladium(II) complex with dichloromethane (170 mg, 208 μmol), and cesium carbonate (677 mg, 2.08 mmol) in a solution of 1,4-dioxane (10 mL) and H₂O (1.0 mL) was degassed with N₂ gas for 5 minutes. The vial was capped, and the mixture was heated and stirred at 90° C. After stirring for 19 hours, the reaction mixture was partitioned between EA and H₂O. The organic phase was collected, and the aqueous layer was extracted with EA (2×). The organic layers were combined, washed with brine, and concentrated under reduced pressure. The residue was purified by SiO₂ chromatography (EA/hexanes) to give 335 mg (78%) of tert-butyl (R)-3-(3-(3-methoxyphenyl)-1H-pyrazolo[3,4-b]pyridin-1-yl)piperidine-1-carboxylate (INT 20B) as a white foam. LCMS-ESI (m/z) calculated for C₂₃H₂₈BN₄O₃: 408.5; found 409.2 [M+H]⁺, t_{R}=0.437 min (Method 11).

### Step 20-3. Synthesis of (R)-3-(3-methoxyphenyl)-1-(piperidin-3-yl)-1H-pyrazolo[3,4-b]pyridine (INT 20C)

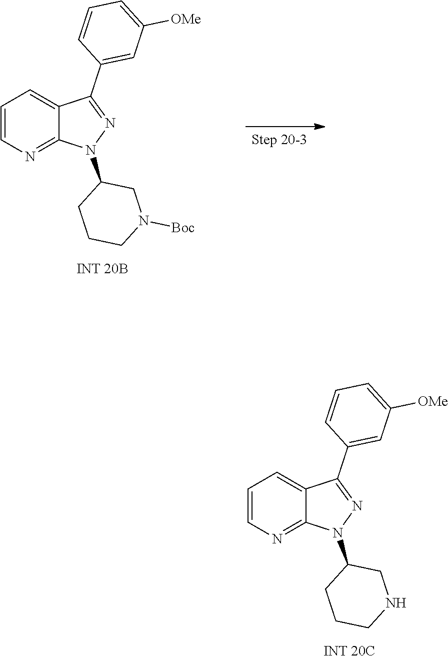 [SMILES: COc1cccc(-c2nn([C@@H]3CCCN(C(=O)OC(C)(C)C)C3)c3ncccc23)c1 ; COc1cccc(-c2nn([C@@H]3CCCNC3)c3ncccc23)c1]

To a 20 mL vial containing a stirring solution of INT 20B (329 mg, 805 μmol) in 1,4-dioxane (6 mL) was added a solution of hydrogen chloride in 1,4-dioxane (294 mg, 2.01 mL, 4.0 molar, 8.05 mmol). After stirring at 50° C. for 19 h, the reaction mixture was filtered. The filter cake was washed with diethyl ether and dried to provide 293 mg (100%) of crude (R)-3-(3-methoxyphenyl)-1-(piperidin-3-yl)-1H-pyrazolo[3,4-b]pyridine (INT 20C) that was used in the next step without further purification. LCMS-ESI (m/z) calculated for C₁₈H₂₁ClN₄O: 308.2; found 309.2 [M+H]⁺, t_{R}=0.559 min (Method 11).

### Step 20-4. Synthesis of (R)—N-(4-(dimethylamino)phenyl)-3-(3-(3-methoxyphenyl)-1H-pyrazolo[3,4-b]pyridin-1-yl)piperidine-1-carboxamide (Compound 20-1)

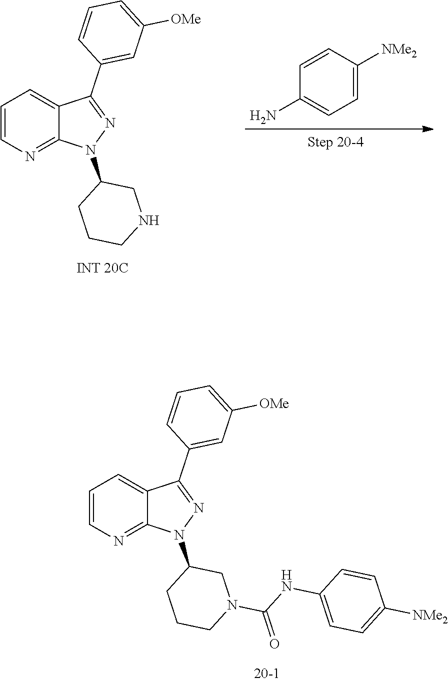 [SMILES: COc1cccc(-c2nn([C@@H]3CCCNC3)c3ncccc23)c1 ; CN(C)c1ccc(N)cc1 ; COc1cccc(-c2nn([C@@H]3CCCN(C(=O)Nc4ccc(N(C)C)cc4)C3)c3ncccc23)c1]

A vial containing a mixture of N,N-dimethyl-p-phenylenediamine (28 mg, 0.20 mmol) in ACN (2 mL) was charged with DIEA (0.18 mL, 1.0 mmol). The mixture was cooled to 0° C., and triphosgene (60 mg, 0.20 mmol) was charged in one portion. The reaction mixture was stirred at 0° C. for 10 minutes, then charged with INT 20C (70 mg, 0.20 mmol) and additional DIEA (2.5 eq.). After stirring for 1.5 h at 50° C., the reaction mixture was passed through a syringe filter into a test tube, and the filtrate was directly loaded/purified by prep HPLC. The relevant fractions were directly lyophilized to produce 47.8 mg (50%) of (R)—N-(4-(dimethylamino)phenyl)-3-(3-(3-methoxyphenyl)-1H-pyrazolo[3,4-b]pyridin-1-yl)piperidine-1-carboxamide (Compound 20-1) as a white solid. LCMS-ESI (m/z) calculated for C₂₇H₃₀N₆O₂: 470.6; found 471.0 [M+H]⁺, t_{R}=6.16 min (Method 1). ¹H NMR (400 MHZ, DMSO-D₆) δ 8.59-8.49 (m, 2H), 8.23 (s, 1H), 7.53 (dt, J=1.2, 7.7 Hz, 1H), 7.44 (dd, J=1.5, 2.7 Hz, 1H), 7.39 (t, J=7.9 Hz, 1H), 7.28 (dd, J=4.5, 8.1 Hz, 1H), 7.20-7.11 (m, 2H), 6.96 (ddd, J=1.0, 2.6, 8.3 Hz, 1H), 6.61-6.53 (m, 2H), 4.95-4.83 (m, 1H), 4.28 (dd, J=4.1, 12.8 Hz, 1H), 4.10 (d, J=13.4 Hz, 1H), 3.79 (s, 3H), 3.35 (dd, J=10.7, 12.8 Hz, 1H), 2.90 (t, J=11.9 Hz, 1H), 2.74 (s, 6H), 2.30-2.15 (m, 1H), 2.11 (d, J=12.3 Hz, 1H), 1.85 (d, J=13.4 Hz, 1H), 1.62 (d, J=12.9 Hz, 1H).

The compound listed in Table 20 were made using the procedures of Scheme 20.

TABLE 20

| | | Purity | | | | |
|---|---|---|---|---|---|---|
| | Cpd | RT | | observed | | Purity |
| Structure | No. | (min) | MW | m/z | Ion | Method |
| 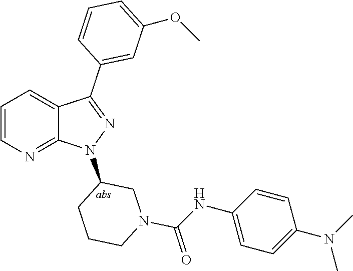 [SMILES: COc1cccc(-c2nn([C@@H]3CCCN(C(=O)Nc4ccc(N(C)C)cc4)C3)c3ncccc23)c1] | 20-1 | 6.161 | 470.2 | 471 | [M + H]⁺ | 1 |
| 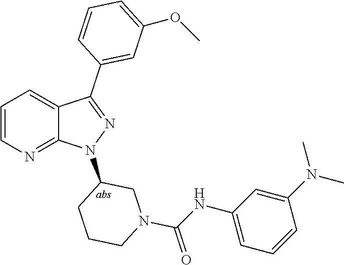 [SMILES: COc1cccc(-c2nn([C@@H]3CCCN(C(=O)Nc4cccc(N(C)C)c4)C3)c3ncccc23)c1] | 20-2 | 7.231 | 470.2 | 471 | [M + H]⁺ | 1 |
| 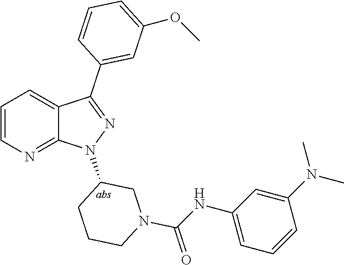 [SMILES: COc1cccc(-c2nn([C@H]3CCCN(C(=O)Nc4cccc(N(C)C)c4)C3)c3ncccc23)c1] | 20-3 | 7.204 | 470.2 | 471.2 | [M + H]⁺ | 1 |
| 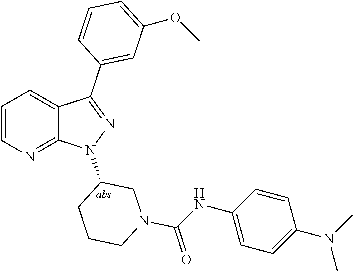 [SMILES: COc1cccc(-c2nn([C@H]3CCCN(C(=O)Nc4ccc(N(C)C)cc4)C3)c3ncccc23)c1] | 20-4 | 6.146 | 470.2 | 471.2 | [M + H]⁺ | 1 |

### Example 21

### Synthesis of Compound 21-1 and Other Representative Compounds

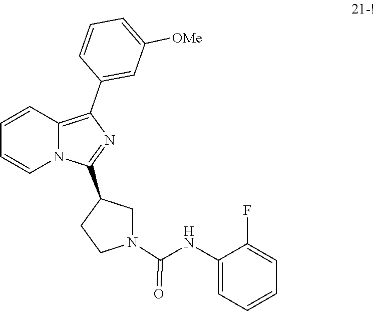 [SMILES: COc1cccc(-c2nc([C@@H]3CCN(C(=O)Nc4ccccc4F)C3)n3ccccc23)c1]

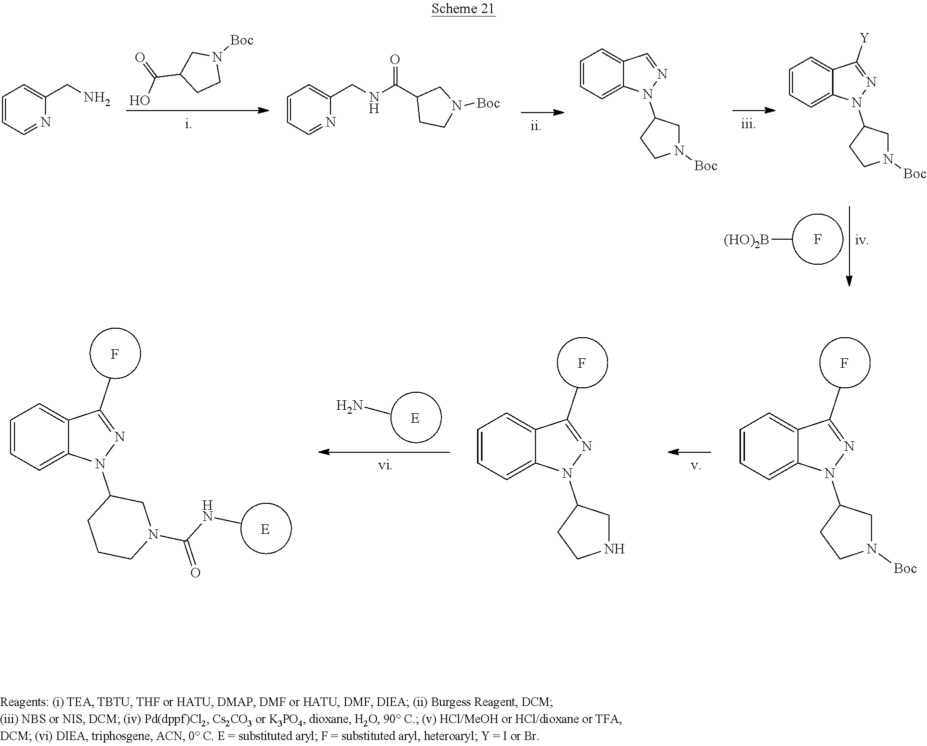

### Step 21-1. Synthesis of tert-butyl (R)-3-(benzylcarbamoyl)pyrrolidine-1-carboxylate (INT 21A)

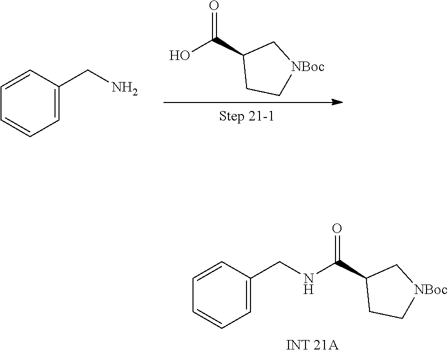 [SMILES: NCc1ccccc1 ; CC(C)(C)OC(=O)N1CC[C@@H](C(=O)O)C1 ; CC(C)(C)OC(=O)N1CC[C@@H](C(=O)NCc2ccccc2)C1]

To a solution of (R)-1-(tert-butoxycarbonyl)pyrrolidine-3-carboxylic acid (2.19 g, 10.17 mmol) in DMF (8 mL) at rt was added HATU (3.87 g, 10.17 mmol). After stirring for 30 min, phenylmethanamine (1.00 g, 9.3 mmol) and DIEA (4.89 g, 37.9 mmol) were added. After stirring at rt for 18 h, the reaction mixture was diluted with aqueous NaHCO₃ and extracted with EA. The organic layer was washed with brine, dried (Na₂SO₄), filtered and concentrated. The resulting residue was purified by SiO₂ chromatography (EA/hexane) to afford 1.1 g (39%) of tert-butyl (R)-3-(benzylcarbamoyl)pyrrolidine-1-carboxylate (INT 21A) as an oil. LCMS-ESI (m/z) calculated for C₁₇H₂₄N₂O₂: 304.4; found 306.1 [M+H]⁺, t_{R}=5.23 min (Method 2).

### Step 21-2. Synthesis of tert-butyl (R)-3-(imidazo[1,5-a]pyridin-3-yl)pyrrolidine-1-carboxylate (INT 21B)

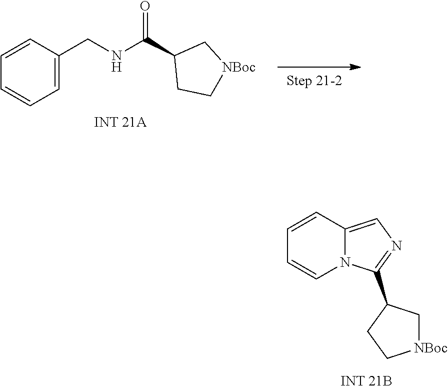 [SMILES: CC(C)(C)OC(=O)N1CC[C@@H](C(=O)NCc2ccccc2)C1 ; CC(C)(C)OC(=O)N1CC[C@@H](c2ncc3ccccn23)C1]

To a solution of INT 21A (1.10 g, 3.6 mmol) in DCM (20 mL) was added Burgess reagent (944 mg, 3.96 mmol). After the reaction was stirred at rt for 18 h, it was concentrated onto SiO₂ and purified using SiO₂ chromatography (EA/hexane) to yield 500 mg (48%) of tert-butyl (R)-3-(imidazo[1,5-a]pyridin-3-yl)pyrrolidine-1-carboxylate (INT 21B). LCMS-ESI (m/z) calculated for C₁₆H₂₁N₃O₂: 287.3; found 287.0 [M+H]⁺, t_{R}=5.10 min (Method 2).

### Step 21-3. Synthesis of tert-butyl (R)-3-(1-bromoimidazo[1,5-a]pyridin-3-yl)pyrrolidine-1-carboxylate (INT 21C)

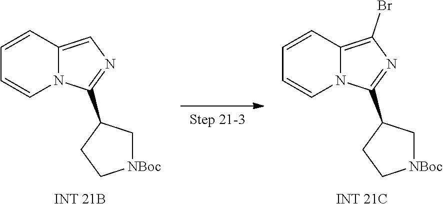 [SMILES: CC(C)(C)OC(=O)N1CC[C@@H](c2ncc3ccccn23)C1 ; CC(C)(C)OC(=O)N1CC[C@@H](c2nc(Br)c3ccccn23)C1]

To a solution of INT 21B (500 mg, 1.74 mmol) in DCM (10 mL) was added NBS (340.7 mg, 1.91 mmol). After the reaction was stirred at 25° C. for 1 h, the mixture was concentrated onto SiO₂ gel and purified by SiO₂ chromatography (EA, hexane) to provide 320 mg (50%) of tert-butyl (R)-3-(1-bromoimidazo[1,5-a]pyridin-3-yl)pyrrolidine-1-carboxylate (INT 21C). LCMS-ESI (m/z) calculated for C₁₆H₂₀BrN₃O₂: 365.0; found 367.7 [M+H]⁺, t_{R}=6.49 min (Method 2).

### Step 21-4. Synthesis of tert-butyl (R)-3-(1-(3-methoxyphenyl)imidazo[1,5-a]pyridin-3-yl)pyrrolidine-1-carboxylate (INT 21D)

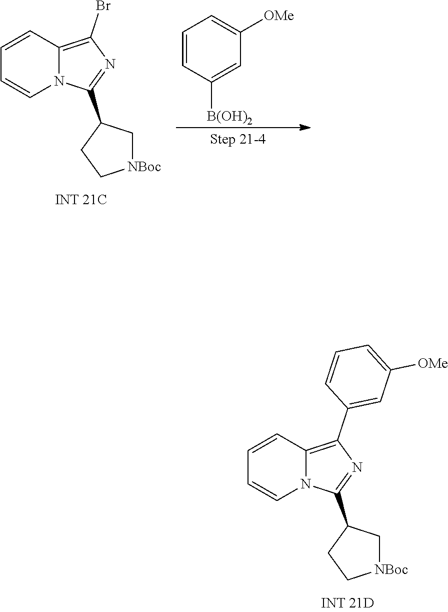 [SMILES: CC(C)(C)OC(=O)N1CC[C@@H](c2nc(Br)c3ccccn23)C1 ; COc1cccc(B(O)O)c1 ; COc1cccc(-c2nc([C@@H]3CCN(C(=O)OC(C)(C)C)C3)n3ccccc23)c1]

To a solution of INT 21C (320 mg, 0.87 mmol) in dioxane (5 mL) and H₂O (0.1 mL) in a sealable tube were added (3-methoxyphenyl) boronic acid (265.5 mg, 1.74 mmol), K₃PO₄ (556.4 mg, 2.62 mmol) and Pd(dppf)Cl₂ (63.9 mg, 0.09 mmol) under an N₂ atmosphere. The tube was sealed, and the reaction mixture was stirred at 100° C. for 1 h. The reaction mixture was diluted with EA, washed with H₂O, saturated aqueous NaHCO₃ and brine, then dried (Na₂SO₄), filtered, concentrated and purified by SiO₂ chromatography (EA/hexane) to produce 310 mg (90%) of tert-butyl (R)-3-(1-(3-methoxyphenyl) imidazo[1,5-a]pyridin-3-yl)pyrrolidine-1-carboxylate (IND 21D) as an off-white solid. LCMS-ESI (m/z) calculated for C₂₃H₂₇N₃O₃: 393.2; found 393.2 [M+H]⁺, t_{R}=6.49 min (Method 2).

### Step 21-5. Synthesis of (R)-1-(3-methoxyphenyl)-3-(pyrrolidin-3-yl)imidazo[1,5-a]pyridine (INT 21E)

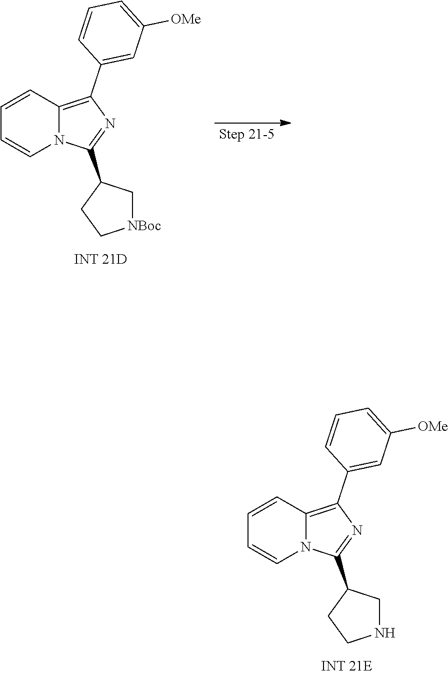 [SMILES: COc1cccc(-c2nc([C@@H]3CCN(C(=O)OC(C)(C)C)C3)n3ccccc23)c1 ; COc1cccc(-c2nc([C@@H]3CCNC3)n3ccccc23)c1]

To a solution of INT 21D (310 mg, 0.79 mmol) in DCM (4 mL) was added TFA (2 mL). After stirring for 3 h at rt, the reaction mixture was concentrated, diluted with EA, and washed with saturated aqueous NaHCO₃ and brine, then dried (Na₂SO₄), filtered, and concentrated to produce 100 mg (44%) of (R)-1-(3-methoxyphenyl)-3-(pyrrolidin-3-yl)imidazo[1,5-a]pyridine (INT 21E) as an oil. LCMS-ESI (m/z) calculated for C₁₈H₁₉N₃O: 293.1; found 294.0 [M+H]⁺, t_{R}=5.46 min (Method 2).

### Step 21-6. Synthesis of (R)—N-(2-fluorophenyl)-3-(1-(3-methoxyphenyl)imidazo[1,5-a]pyridin-3-yl)pyrrolidine-1-carboxamide (Compound 21-1)

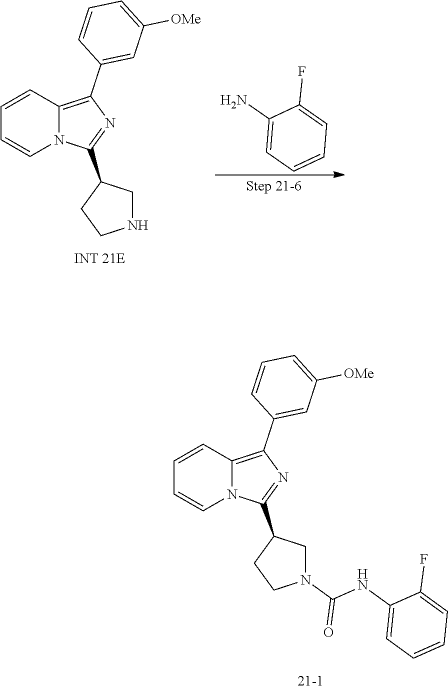 [SMILES: COc1cccc(-c2nc([C@@H]3CCNC3)n3ccccc23)c1 ; Nc1ccccc1F ; COc1cccc(-c2nc([C@@H]3CCN(C(=O)Nc4ccccc4F)C3)n3ccccc23)c1]

To a solution of 2-fluoroaniline (14.2 mg, 0.13 mmol) and triphosgene (14.4 mg, 0.05 mmol) in DCM (5 mL) was added DIEA (165 mg, 1.28 mmol), dropwise at 0° C. After stirring for 10 min at 0° C., INT 21E (37.5 mg, 0.13 mmol) was added. After 1 h, the reaction mixture was diluted with H₂O and extracted with DCM (3×). The combined organic layers were dried (NaSO₄), concentrated, purified by SiO₂ chromatography (EA/hexane) and then reverse-phase chromatography to produce 17 mg (31%) of (R)—N-(2-fluorophenyl)-3-(1-(3-methoxyphenyl)imidazo[1,5-a]pyridin-3-yl) pyrrolidine-1-carboxamide (Compound 21-1). LCMS-ESI (m/z) calculated for C₂₅H₂₃FN₄O₂: 430.5; found 431 [M+H]⁺, t_{R}=11.28 min (Method 2). ¹H NMR (400 MHZ, DMSO-D₆) δ=8.37 (d, J=7.2 Hz, 1H), 7.98 (s, 1H), 7.91 (d, J=9.3 Hz, 1H), 7.55 (dq, J=2.9, 3.6, 7.8 Hz, 1H), 7.46 (d, J=7.7 Hz, 1H), 7.41 (t, J=2.0 Hz, 1H), 7.35 (t, J=7.9 Hz, 1H), 7.23-7.15 (m, 1H), 7.11 (dq, J=3.7, 4.3, 6.8 Hz, 2H), 6.91 (dd, J=6.3, 9.3 Hz, 1H), 6.82 (dd, J=2.6, 8.1 Hz, 1H), 6.76 (t, J=6.8 Hz, 1H), 4.07 (t, J=7.7 Hz, 1H), 4.00 (q, J=8.3 Hz, 1H), 3.82 (s, 3H), 3.75 (t, J=14.6 Hz, 2H), 3.55 (dt, J=6.5, 13.3 Hz, 1H), 2.46 (d, J=7.9 Hz, 1H), 2.29 (t, J=10.3 Hz, 1H).

The compounds listed in Table 21 were made using the procedures of Scheme 21.

TABLE 21

| | | Purity | | | | |
|---|---|---|---|---|---|---|
| | Cpd | RT | | observed | | Purity |
| Structure | No. | (min) | MW | m/z | Ion | Method |
| 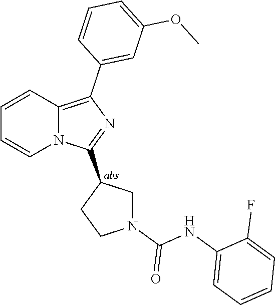 [SMILES: COc1cccc(-c2nc([C@@H]3CCN(C(=O)Nc4ccccc4F)C3)n3ccccc23)c1] | 21-1 | 11.2 | 430.2 | 431 | [M + H]⁺ | 2 |
| 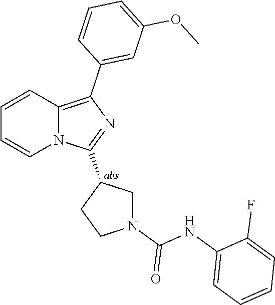 [SMILES: COc1cccc(-c2nc([C@H]3CCN(C(=O)Nc4ccccc4F)C3)n3ccccc23)c1] | 21-2 | 11.12 | 430.2 | 431.1 | [M + H]⁺ | 2 |
| 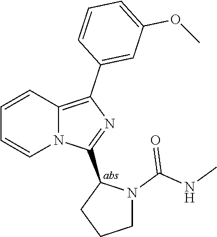 [SMILES: CNC(=O)N1CCC[C@H]1c1nc(-c2cccc(OC)c2)c2ccccn12] | 21-3 | 9.49 | 350.2 | 351.5 | [M + H]⁺ | 2 |
| 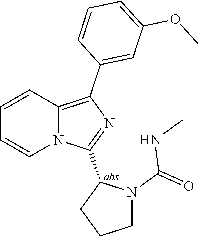 [SMILES: CNC(=O)N1CCC[C@@H]1c1nc(-c2cccc(OC)c2)c2ccccn12] | 21-4 | 9.54 | 350.2 | 351.5 | [M + H]⁺ | 2 |
| 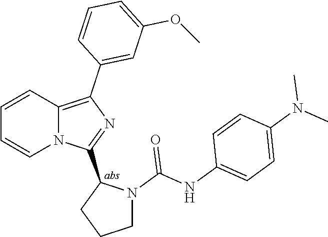 [SMILES: COc1cccc(-c2nc([C@@H]3CCCN3C(=O)Nc3ccc(N(C)C)cc3)n3ccccc23)c1] | 21-5 | 9.54 | 455.2 | 456.6 | [M + H]⁺ | 2 |
| 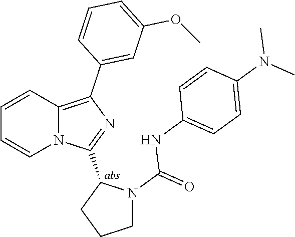 [SMILES: COc1cccc(-c2nc([C@H]3CCCN3C(=O)Nc3ccc(N(C)C)cc3)n3ccccc23)c1] | 21-6 | 9.59 | 455.2 | 456.6 | [M + H]⁺ | 2 |

### Example 22

### Synthesis of Compound 22-1 and Other Representative Compounds

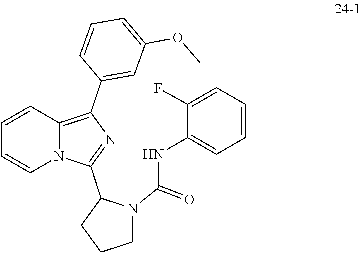 [SMILES: COc1cccc(-c2nc(C3CCCN3C(=O)Nc3ccccc3F)n3ccccc23)c1]

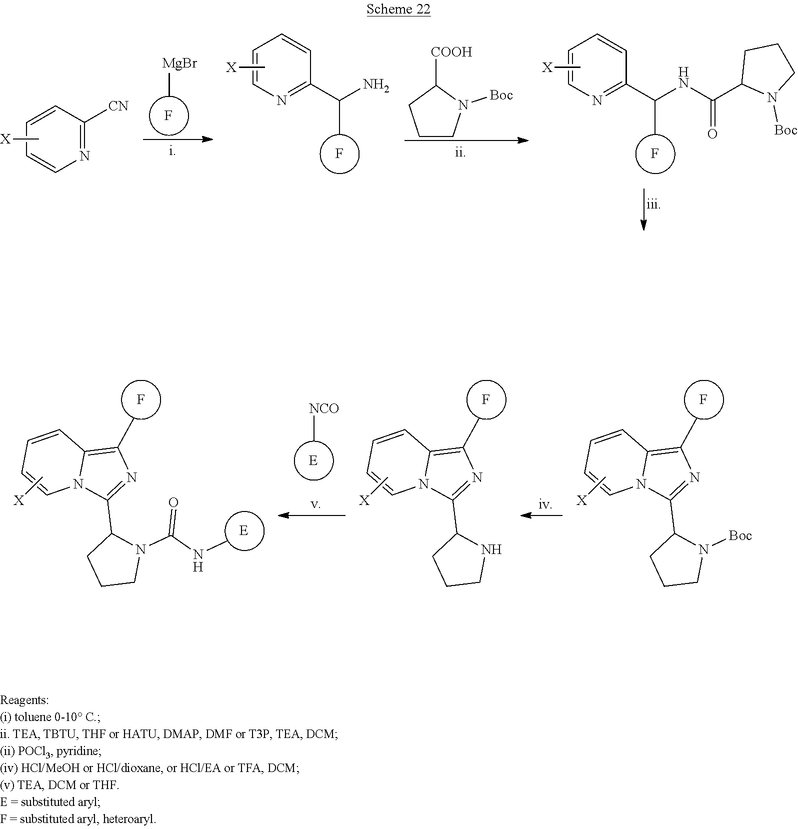

### Step 22-1. Synthesis of (3-methoxyphenyl)(pyridin-2-yl)methanamine (INT 22A)

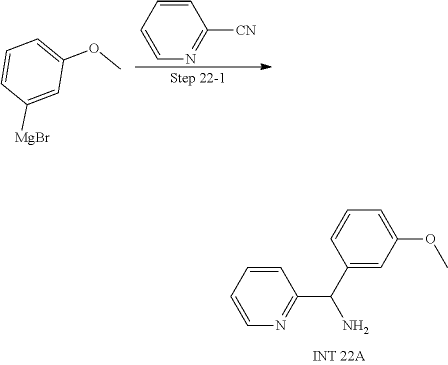

To a solution of pyridine-2-carbonitrile (5 g, 48.03 mmol, 4.63 mL) in toluene (200 mL) was added bromo-(3-methoxyphenyl)magnesium (20.10 mL, 96.05 mmol) at 0° C. The reaction was stirred at 0° C. for 1 h. Then ⁱ-BuOH (100 mL) was added to the mixture at 0° C.˜10° C. followed by NaBH₄ (3.63 g, 96.1 mmol) at 0° C. After the reaction was stirred at 20° C. for 18 h, it was quenched with MeOH (500 mL), concentrated and purified by SiO₂ chromatography (EA/petroleum ether) to produce 6 g (58%) of (3-methoxyphenyl)(pyridin-2-yl)methanamine (INT 22A) as a brown oil. ¹H NMR (400 MHZ, CD₃Cl) δ=3.80 (s, 3H), 5.24 (s, 1H), 6.76-6.83 (m, 1H), 7.00 (s, 1H), 7.07-7.20 (m, 2H), 7.22-7.29 (m, 2H), 7.62 (td, J=7.67, 1.65 Hz, 1H), 8.59 (br d, J=4.65 Hz, 1H).

### Step 22-2. Synthesis of tert-butyl 2-(((3-methoxyphenyl)(pyridin-2-yl)methyl)carbamoyl)pyrrolidine-1-carboxylate (INT 22B)

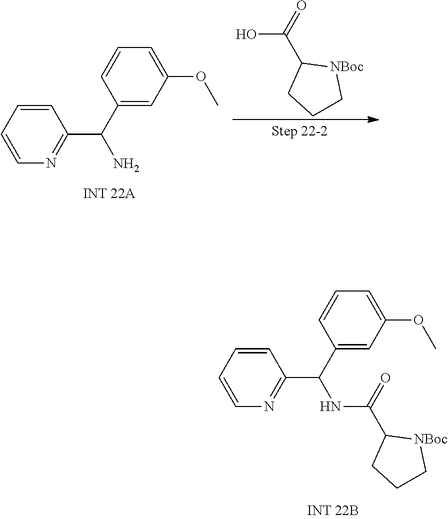 [SMILES: COc1cccc(C(N)c2ccccn2)c1 ; CC(C)(C)OC(=O)N1CCCC1C(=O)O ; COc1cccc(C(NC(=O)C2CCCN2C(=O)OC(C)(C)C)c2ccccn2)c1]

To a solution of INT 22A (2 g, 9.3 mmol) and 1-tert-butoxycarbonylpyrrolidine-2-carboxylic acid (2.41 g, 11.20 mmol) in DCM (20 mL) was added TEA (3.9 mL, 28 mmol) at 20° C. The reaction mixture was cooled to 0° C. and a solution of T₃P (8.33 mL, 14 mmol) in EA (4.2 mL) was added. The reaction mixture was warmed to 20° C. and stirred for 12 h then concentrated in vacuo to give a residue that was purified by prep SiO₂ chromatography (EA/petroleum ether) to provide 2.1 g (55%) of tert-butyl 2-(((3-methoxyphenyl) (pyridin-2-yl) methyl)carbamoyl) pyrrolidine-1-carboxylate (INT 22B) as a yellow oil. ¹H NMR (400 MHz, CD₃Cl) δ=1.21-1.39 (m, 6H), 1.50 (br s, 3H), 1.57-1.64 (m, 1H), 1.82-1.93 (m, 2H), 2.14 (br dd, J=4.89, 2.93 Hz, 1H), 3.34-3.63 (m, 2H), 3.76 (s, 3H), 6.06-6.18 (m, 1H), 6.76 (dd, J=8.19, 1.96 Hz, 1H), 6.90 (br d, J=7.70 Hz, 2H), 7.15-7.23 (m, 2H), 7.27-7.30 (m, 1H), 7.57-7.69 (m, 1H), 8.56 (br s, 1H).

### Step 22-3. Synthesis of tert-butyl 2-(1-(3-methoxyphenyl)imidazo[1,5-a]pyridin-3-yl)pyrrolidine-1-carboxylate (INT 22C)

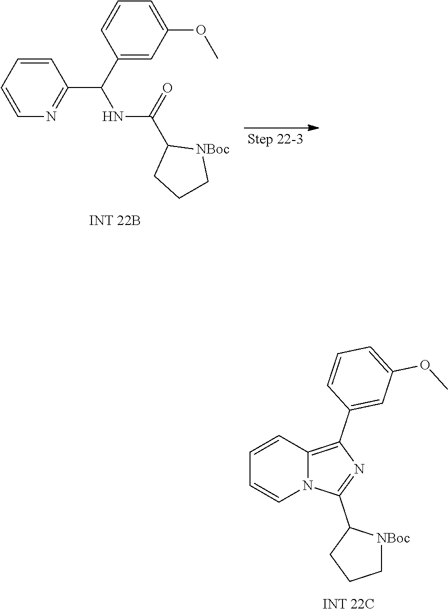 [SMILES: COc1cccc(C(NC(=O)C2CCCN2C(=O)OC(C)(C)C)c2ccccn2)c1 ; COc1cccc(-c2nc(C3CCCN3C(=O)OC(C)(C)C)n3ccccc23)c1]

To a solution of INT 22B (2.1 g, 5.1 mmol) in DCM (20 mL) was added pyridine (2.47 mL, 30.6 mmol) at 20° C. The reaction was cooled to 0° C. and POCl₃ (570 μL, 6.12 mmol) was added. After stirring for 18 h, the mixture was concentrated, and purified by SiO₂ chromatography (EA/petroleum ether) to provide 1.1 g (55%) of tert-butyl 2-(1-(3-methoxyphenyl)imidazo[1,5-a]pyridin-3-yl) pyrrolidine-1-carboxylate (INT 22C) as a yellow oil. LCMS-ESI (m/z) calculated for C₂₃H₂₇N₃O₃: 393.2; found 394.2 [M+H]⁺, t_{R}=0.71 min (Method 4). ¹H NMR (400 MHZ, CD₃Cl) δ=0.97 (br s, 5H), 1.21 (br d, J=19.20 Hz, 2H), 1.31-1.43 (m, 4H), 1.72-2.03 (m, 2H), 2.14-2.36 (m, 2H), 3.81 (s, 3H), 6.46-6.55 (m, 1H), 6.67 (dd, J=9.23, 6.17 Hz, 1H), 7.26 (br d, J=1.83 Hz, 2H), 7.36 (br d, J=6.72 Hz, 2H), 7.66 (br t, J=7.64 Hz, 2H), 8.56 (br d, J=4.28 Hz, 1H).

### Step 22-4. Synthesis of 1-(3-methoxyphenyl)-3-(pyrrolidin-2-yl)imidazo[1,5-a]pyridine (INT 22D)

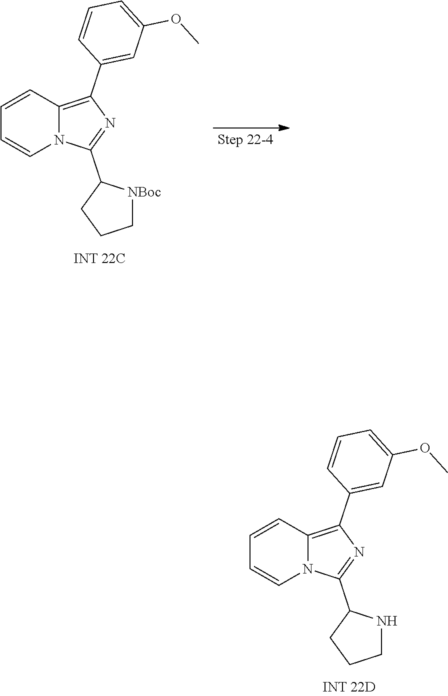 [SMILES: COc1cccc(-c2nc(C3CCCN3C(=O)OC(C)(C)C)n3ccccc23)c1 ; COc1cccc(-c2nc(C3CCCN3)n3ccccc23)c1]

A solution of INT 22C (1.1 g, 2.8 mmol) in 4M HCl/EA (15 mL) was stirred at 20° C. for 3 h. The mixture was concentrated to provide crude 500 mg (54%) of 1-(3-methoxyphenyl)-3-(pyrrolidin-2-yl)imidazo[1,5-a]pyridine (INT 22D) as a white solid that was used in the next step without further purification. TLC (10:1 EA/MeOH), t_{R}=0.03.

### Step 22-5. Synthesis of N-(2-fluorophenyl)-2-(1-(3-methoxyphenyl)imidazo[1,5-a]pyridin-3-yl)pyrrolidine-1-carboxamide (Compound 22-1)

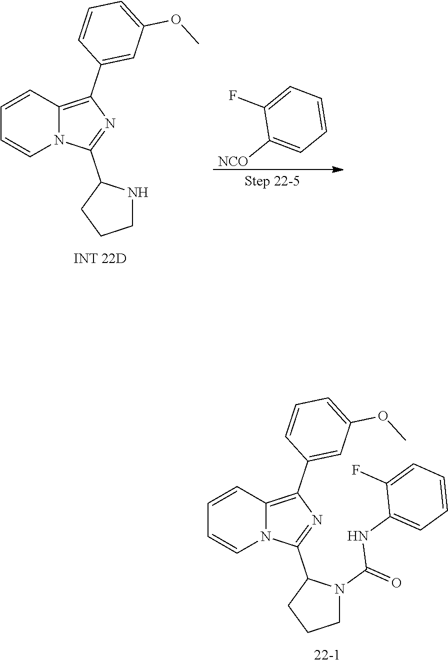

To a solution of INT 22D (500 mg, 1.70 mmol) in THF (10 mL) were added 1-fluoro-2-isocyanato-benzene (350.5 mg, 2.56 mmol) and TEA (1.42 mL, 10.23 mmol). After the reaction was stirred at 20° C. for 18 h, the mixture was concentrated and purified by prep-HPLC chromatography (H₂O (0.05% NH₃H₂O+10 mM NH₄HCO₃)/ACN) to provide 200 mg (27%) of N-(2-fluorophenyl)-2-(1-(3-methoxyphenyl) imidazo[1,5-a]pyridin-3-yl)pyrrolidine-1-carboxamide (Compound 22-1) as a yellow solid. LCMS-ESI (m/z) calculated for C₂₅H₂₃FN₄O₂: 430.1; found 431.2 [M+H]⁺, t_{R}=0.861 min (Method 4).

The compounds listed in Table 22 were made using the procedures of Scheme 22.

TABLE 22

| | | Purity | | | | |
|---|---|---|---|---|---|---|
| | Cpd | RT | | observed | | Purity |
| Structure | No. | (min) | MW | m/z | Ion | Method |
| 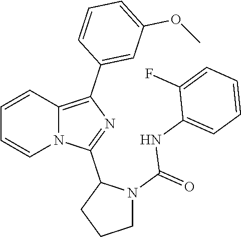 [SMILES: COc1cccc(-c2nc(C3CCCN3C(=O)Nc3ccccc3F)n3ccccc23)c1] | 22-1 | 0.861 | 430.2 | 431.2 | [M + H]⁺ | 4 |

### Example 23

### Synthesis of Compound 23-1 and Other Representative Compounds

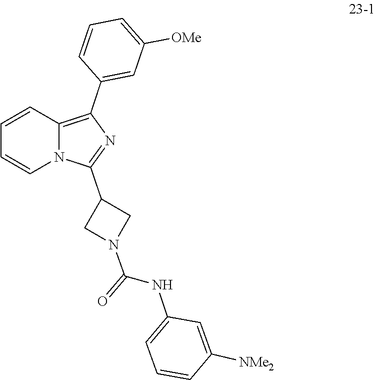 [SMILES: COc1cccc(-c2nc(C3CN(C(=O)Nc4cccc(N(C)C)c4)C3)n3ccccc23)c1]

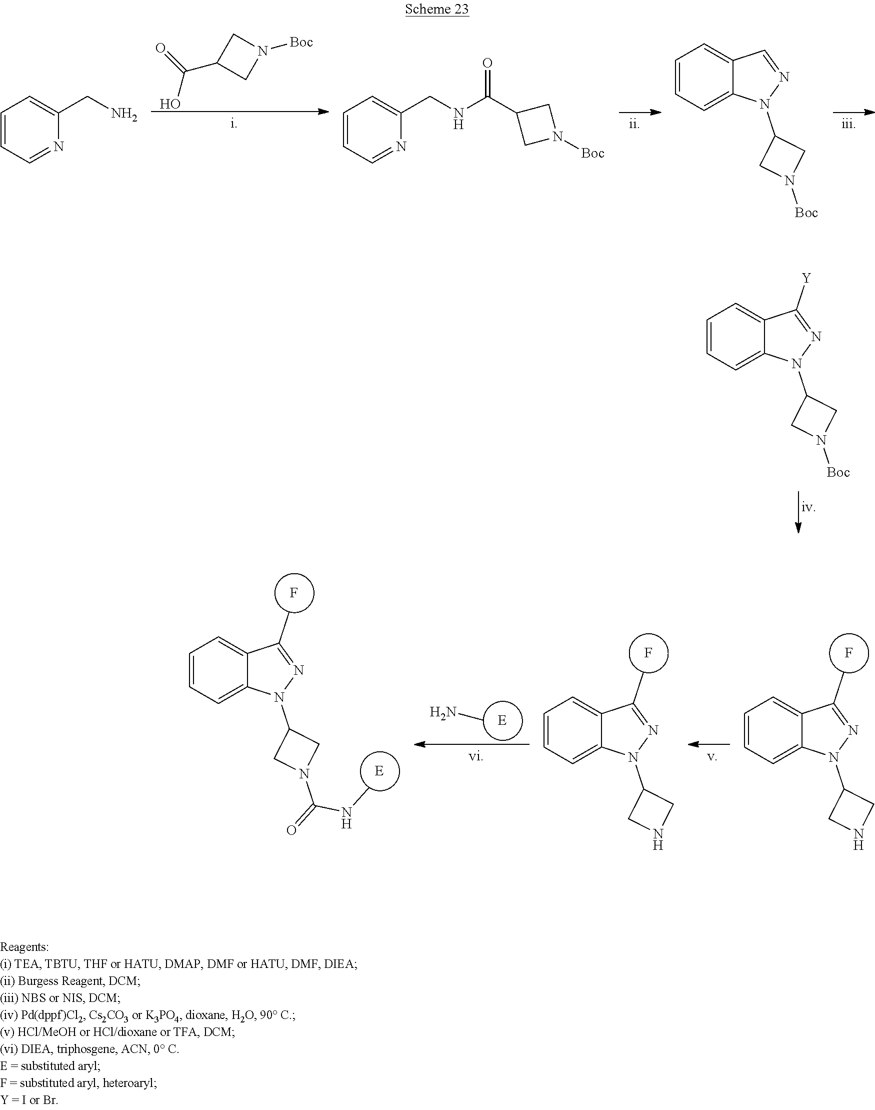

### Step 23-1. Synthesis of tert-butyl 3-((pyridin-2-ylmethyl)carbamoyl)azetidine-1-carboxylate (INT 23A)

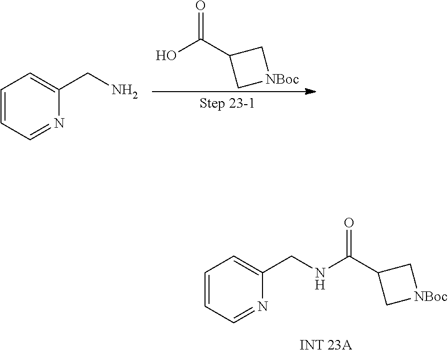 [SMILES: NCc1ccccn1 ; CC(C)(C)OC(=O)N1CC(C(=O)O)C1 ; CC(C)(C)OC(=O)N1CC(C(=O)NCc2ccccn2)C1]

To a stirring solution of 1-(tert-butoxycarbonyl) azetidine-3-carboxylic acid (1.0 g, 5.0 mmol) and DIEA (3.5 mL, 19.9 mmol) in DCM (15 mL) was added HATU (1.89 g, 4.97 mmol). After stirring for 10 minutes, the reaction mixture was charged with 2-(aminomethyl) pyridine (512 μL, 4.97 mmol). The resulting mixture was allowed to stir at room temperature for 1 h. The reaction mixture was diluted with DCM and washed with saturated NH₄Cl, saturated NaHCO₃, and brine. The organic layer was dried (Na₂SO₄) and concentrated to yield 2.59 g (179%) of crude tert-butyl 3-((pyridin-2-ylmethyl) carbamoyl) azetidine-1-carboxylate (INT 23A) as a purple oil that was taken forward to the next step without further purification. LCMS-ESI (m/z) calculated for C₁₅H₂₁N₃O₃: 291.35; found 292.2 [M+H]⁺, t_{R}=0.45 min (Method 11).

### Step 23-2. Synthesis of tert-butyl 3-(imidazo[1,5-a]pyridin-3-yl)azetidine-1-carboxylate (INT 23B)

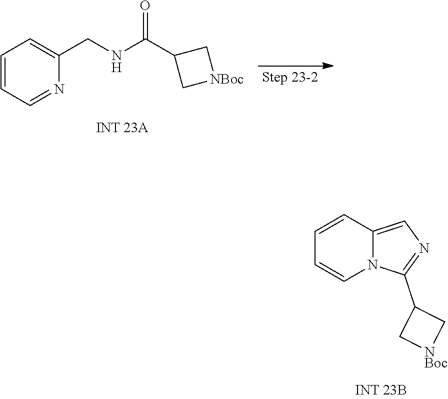 [SMILES: CC(C)(C)OC(=O)N1CC(C(=O)NCc2ccccn2)C1 ; CC(C)(C)OC(=O)N1CC(c2ncc3ccccn23)C1]

To a solution of INT 23A (1.44 g, 4.94 mmol) in DCM (20 mL) at 0° C. was added Burgess reagent (1.18 g, 4.94 mmol). The resulting mixture was allowed to stir for 18 h at rt. The reaction mixture was directly purified by SiO₂ chromatography (MeOH/DCM). The relevant fractions were combined and concentrated under reduced pressure to yield 1.46 g (108%) of tert-butyl 3-(imidazo[1,5-a]pyridin-3-yl)azetidine-1-carboxylate (INT 23B) as a yellow-green syrup that was used without further purification. LCMS-ESI (m/z) calculated for C₁₅H₁₉N₃O₂: 273.3; found 274.2 [M+H]⁺, t_{R}=0.568 min (Method 11).

### Step 23-3. Synthesis of tert-butyl 3-(1-bromoimidazo[1,5-a]pyridin-3-yl)azetidine-1-carboxylate (INT 23C)

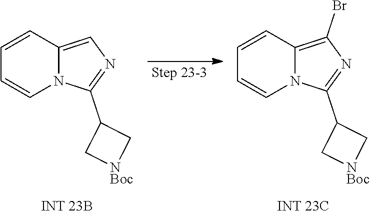 [SMILES: CC(C)(C)OC(=O)N1CC(c2ncc3ccccn23)C1 ; CC(C)(C)OC(=O)N1CC(c2nc(Br)c3ccccn23)C1]

To a stirring solution of INT 23B (1.35 g, 4.94 mmol) in DCM (16 mL) was added N-bromosuccinimide (879 mg, 4.94 mmol). After stirring for 17 h, the reaction mixture was directly purified by flash column chromatography (EA/hexanes) to provide 457 mg (26%) of tert-butyl 3-(1-bromoimidazo[1,5-a]pyridin-3-yl)azetidine-1-carboxylate (INT 23C). LCMS-ESI (m/z) calculated for C₁₅H₁₈BrN₃O₂: 352.23; found 353.9 [M+H]⁺. (Method 11). ¹H NMR (400 MHz, DMSO-D₆) δ 8.15 (dt, J=1.1, 7.1 Hz, 1H), 7.39 (dt, J=1.2, 9.2 Hz, 1H), 6.90 (ddd, J=0.9, 6.4, 9.3 Hz, 1H), 6.76 (ddd, J=1.2, 6.4, 7.5 Hz, 1H), 4.32 (dt, J=6.2, 9.3 Hz, 3H), 4.09 (s, 2H), 1.41 (s, 9H).

### Step 23-4. Synthesis of tert-butyl 3-(1-(3-methoxyphenyl)imidazo[1,5-a]pyridin-3-yl)azetidine-1-carboxylate (INT 23D)

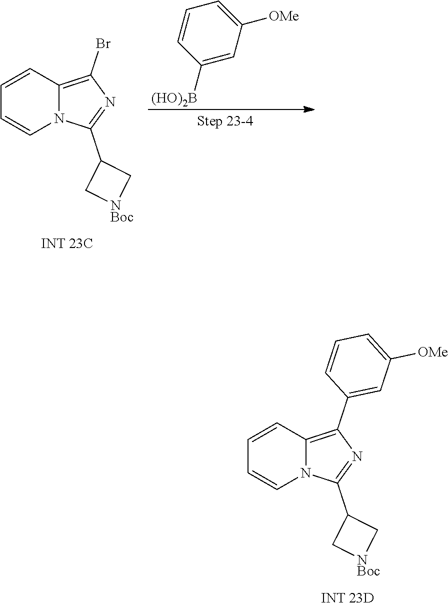 [SMILES: CC(C)(C)OC(=O)N1CC(c2nc(Br)c3ccccn23)C1 ; COc1cccc(B(O)O)c1 ; COc1cccc(-c2nc(C3CN(C(=O)OC(C)(C)C)C3)n3ccccc23)c1]

A 20 mL pressure vial containing a mixture INT 23C (453 mg, 1.29 mmol), 3-methoxyphenylboronic acid (215 mg, 1.41 mmol), [1,1′-Bis(diphenylphosphino) ferrocene] dichloropalladium (II) Complex with dichloromethane (210 mg, 257 μmol), and cesium carbonate (838 mg, 2.57 mmol) in a solution of 1,4-dioxane (7 mL) and H₂O (0.7 mL) was degassed with N₂ gas for 10 minutes. The vial was capped, and the mixture was heated/stirred at 90° C. After stirring for 19 hours, the reaction mixture was partitioned between EA and H₂O. The organic phase was collected and the aqueous layer was extracted with EA (2×). The organic layers were combined, washed with brine, and concentrated under reduced pressure. The residue was dissolved in DCM (12 mL) and purified by SiO₂ (EA/hexanes). The relevant fractions were combined and concentrated under reduced pressure to yield 330 mg (68%) of tert-butyl 3-(1-(3-methoxyphenyl)imidazo[1,5-a]pyridin-3-yl)azetidine-1-carboxylate (INT 23D) as a yellow foam. LCMS-ESI (m/z) calculated for C₂₂H₂₅N₃O₃: 379.5; found 380.2 [M+H]⁺, t_{R}=1.055 min (Method 11).

### Step 23-5. Synthesis of 3-(azetidin-3-yl)-1-(3-methoxyphenyl)imidazo[1,5-a]pyridine (INT 23E)

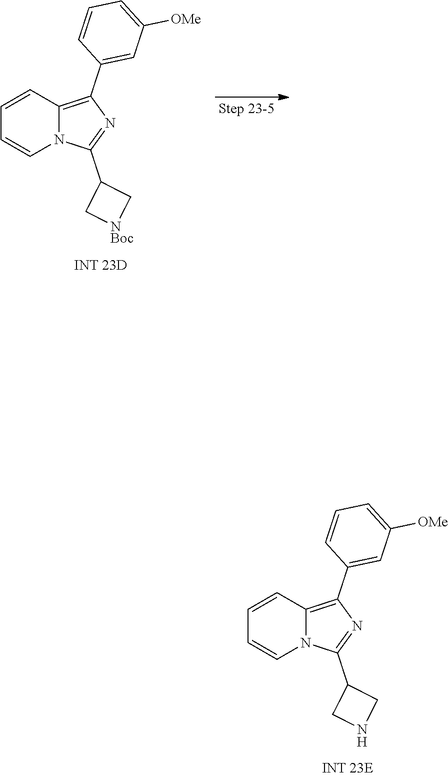 [SMILES: COc1cccc(-c2nc(C3CN(C(=O)OC(C)(C)C)C3)n3ccccc23)c1 ; COc1cccc(-c2nc(C3CNC3)n3ccccc23)c1]

To a solution of INT 23D (325 mg, 856 μmol) in 1,4-Dioxane (5 mL) was added 4M hydrogen chloride in 1,4-dioxane (2.14 mL, 8.56 mmol). After stirring for 17 h at 50° C., the reaction mixture was dissolved in MeOH and concentrated under reduced pressure to yield 366 mg (135%) of crude 3-(azetidin-3-yl)-1-(3-methoxyphenyl)imidazo[1,5-a]pyridine hydrochloride (INT 23E) as a light-brown solid. The product was taken forward to the next step without further purification LCMS-ESI (m/z) calculated for C₁₇H₁₇N₃O: 279.3; found 280.0 [M+H]⁺, t_{R}=0.577 (Method 11).

### Step 23-6. Synthesis of N-(3-(dimethylamino)phenyl)-3-(1-(3-methoxyphenyl) imidazo[1,5-a]pyridin-3-yl)azetidine-1-carboxamide (Compound 23-1)

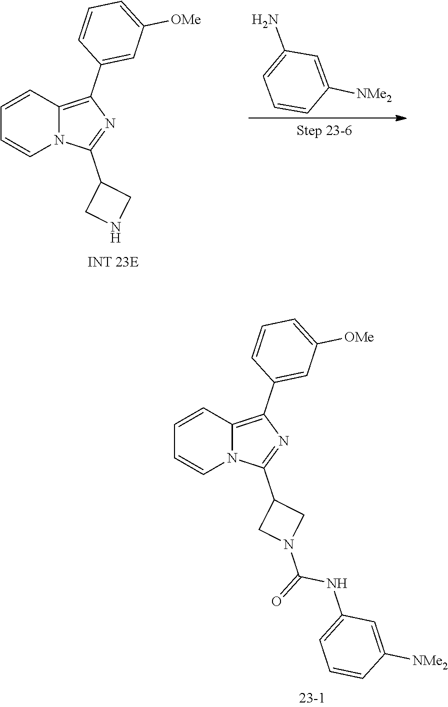 [SMILES: COc1cccc(-c2nc(C3CNC3)n3ccccc23)c1 ; CN(C)c1cccc(N)c1 ; COc1cccc(-c2nc(C3CN(C(=O)Nc4cccc(N(C)C)c4)C3)n3ccccc23)c1]

A solution of N1,N1-dimethylbenzene-1,3-diamine (22 mg, 0.16 mmol) in CAN (2 mL) was charged with DIPEA (0.14 mL, 0.79 mmol). The mixture was cooled to 0° C., and triphosgene (47 mg, 0.16 mmol) was added. The reaction mixture was stirred at 0° C. for 10 minutes, then was charged with INT 23E (50 mg, 0.16 mmol), and DIPEA (2.5 eq.). After stirring for 1.5 h at 50° C., the reaction mixture was passed through a syringe filter into a test tube, and the filtrate was purified by prep HPLC. The relevant fractions were lyophilized to yield 6 mg (9%) of N-(3-(dimethylamino)phenyl)-3-(1-(3-methoxyphenyl)imidazo[1,5-a]pyridin-3-yl)azetidine-1-carboxamide (Compound 23-1) as a pale-yellow solid. LCMS-ESI (m/z) calculated for C₂₆H₂₇N₅O₂: 441.5; found 442.0 [M+H]⁺, t_{R}=5.843 (Method 1). ¹H NMR (400 MHZ, DMSO) δ=8.30 (s, 1H), 8.15-8.07 (m, 1H), 7.86 (dt, J=1.2, 9.3 Hz, 1H), 7.42 (dt, J=1.2, 7.6 Hz, 1H), 7.35 (dd, J=1.5, 2.6 Hz, 1H), 7.28 (t, J=7.9 Hz, 1H), 6.95 (t, J=8.1 Hz, 1H), 6.91-6.79 (m, 3H), 6.79-6.65 (m, 2H), 6.30-6.23 (m, 1H), 4.39 (t, J=7.4 Hz, 2H), 4.36-4.26 (m, 1H), 4.29-4.21 (m, 2H), 3.76 (s, 3H), 2.78 (s, 6H).

The compounds listed in Table 23 were made using the procedures of Scheme 23.

TABLE 23

| | | Purity | | | | |
|---|---|---|---|---|---|---|
| | Cpd | RT | | observed | | Purity |
| Structure | No. | (min) | MW | m/z | Ion | Method |
| 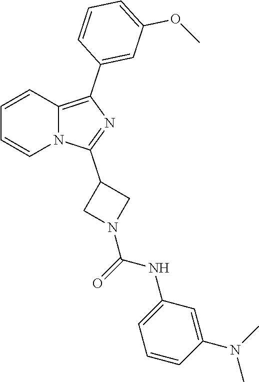 [SMILES: COc1cccc(-c2nc(C3CN(C(=O)Nc4cccc(N(C)C)c4)C3)n3ccccc23)c1] | 23-1 | 5.843 | 441.2 | 442 | [M + H]⁺ | 1 |
| 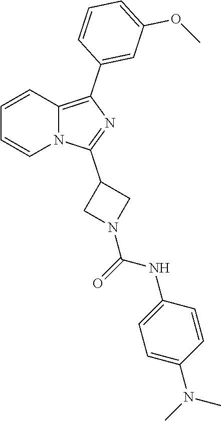 [SMILES: COc1cccc(-c2nc(C3CN(C(=O)Nc4ccc(N(C)C)cc4)C3)n3ccccc23)c1] | 23.2 | 5.19 | 441.2 | 442 | [M + H]⁺ | 1 |

### Example 24

### Synthesis of Compound 24-1 and Other Representative Compounds

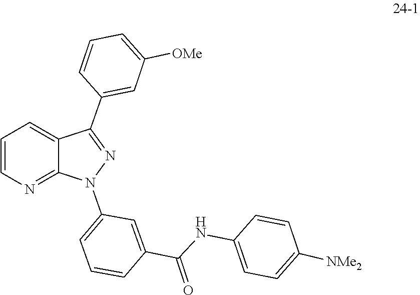 [SMILES: COc1cccc(-c2nn(-c3cccc(C(=O)Nc4ccc(N(C)C)cc4)c3)c3ncccc23)c1]

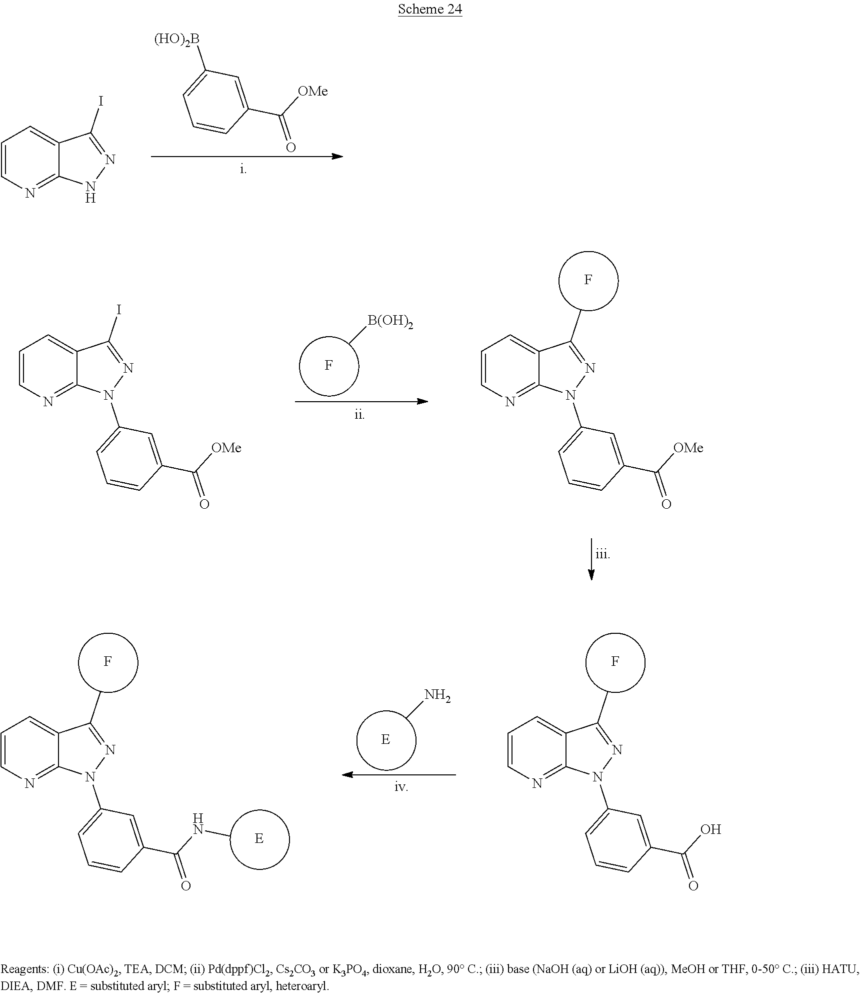

### Step 24-1. Synthesis of methyl 3-(3-iodo-1H-pyrazolo[3,4-b]pyridin-1-yl)benzoate (INT 24A)

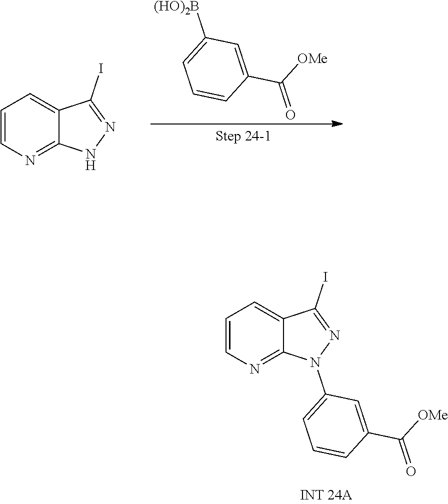 [SMILES: Ic1n[nH]c2ncccc12 ; COC(=O)c1cccc(B(O)O)c1 ; COC(=O)c1cccc(-n2nc(I)c3cccnc32)c1]

To a stirring solution of 3-iodo-1H-pyrazolo[3,4-b]pyridine (300 mg, 1.22 mmol) and 3-methoxycarbonylphenylboronic acid (441 mg, 2.45 mmol) in DCM (5 mL) were added copper (II) acetate (222 mg, 1.22 mmol) and TEA (341 μL, 2.45 mmol). After stirring for 18 h at rt, the reaction mixture was passed through a syringe filter and the filtrate was purified by SiO₂ chromatography (EA/hexanes). The relevant fractions were combined and concentrated under reduced pressure to yield 94 mg (20%) of methyl 3-(3-iodo-1H-pyrazolo[3,4-b]pyridin-1-yl) (INT 24A) as a white solid. LCMS-ESI (m/z) calculated for C₁₄H₁₀IN₃O₂: 379.2; found 380.2 [M+H]⁺, t_{R}=1.152 min (Method 11). ¹H NMR (400 MHZ, DMSO-D₆) δ=8.83 (t, J=1.9 Hz, 1H), 8.78 (dd, J=1.6, 4.6 Hz, 1H), 8.57 (ddd, J=1.1, 2.3, 8.1 Hz, 1H), 8.12 (dd, J=1.6, 8.1 Hz, 1H), 7.94 (dt, J=1.3, 7.8 Hz, 1H), 7.75 (t, J=8.0 Hz, 1H), 7.48 (dd, J=4.6, 8.1 Hz, 1H), 3.93 (s, 3H).

### Step 24-2. Synthesis of methyl 3-(3-(3-methoxyphenyl)-1H-pyrazolo[3,4-b]pyridin-1-yl)benzoate (INT 24B)

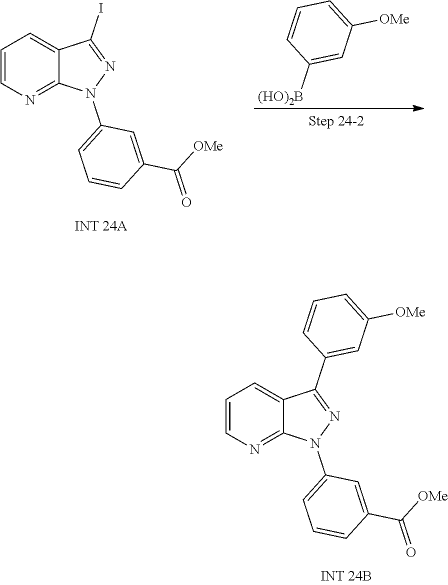 [SMILES: COC(=O)c1cccc(-n2nc(I)c3cccnc32)c1 ; COc1cccc(B(O)O)c1 ; COC(=O)c1cccc(-n2nc(-c3cccc(OC)c3)c3cccnc32)c1]

To a vial containing INT 24A (88 mg, 0.23 mmol), 3-methoxyphenylboronic acid (39 mg, 0.26 mmol), [1,1′-Bis (diphenylphosphino) ferrocene] dichloropalladium(II) complex with dichloromethane (38 mg, 46 μmol), and cesium carbonate (0.15 g, 2 Eq, 0.46 mmol) were added 1,4-Dioxane (3 mL) and H₂O (0.3 mL). The solution was degassed with nitrogen gas for 5 minutes before the vial was capped and the mixture was stirred at 90° C. for 17 h. The reaction mixture was partitioned between EA and H₂O. The organic phase was collected, and the aqueous layer was further extracted with EA (2×). The organic layers were combined, washed with brine, and concentrated under reduced pressure. The residue was dissolved in DCM and purified by SiO₂ chromatography (EA/hexanes). The relevant fractions were combined and concentrated under reduced pressure to yield 67 mg (81%) of methyl 3-(3-(3-methoxyphenyl)-1H-pyrazolo[3,4-b]pyridin-1-yl)benzoate (INT 24B) as a white to off-white solid. LCMS-ESI (m/z) calculated for C₂₁H₁₇N₃O₃: 359.39; found 360.2 [M+H]⁺, t_{R}=1.223 min (Method 11). 1HNMR (400 MHZ, DMSO) δ 9.00 (t, J=1.9 Hz, 1H), 8.79 (dd, J=1.5, 4.6 Hz, 1H), 8.76-8.65 (m, 2H), 7.95 (dt, J=1.4, 7.8 Hz, 1H), 7.77 (t, J=7.9 Hz, 1H), 7.70 (dt, J=1.2, 7.8 Hz, 1H), 7.61 (dd, J=1.5, 2.7 Hz, 1H), 7.57-7.46 (m, 2H), 7.12 (ddd, J=1.0, 2.6, 8.3 Hz, 1H), 3.94 (s, 3H), 3.90 (s, 3H).

### Step 24-3. Synthesis of 3-(3-(3-methoxyphenyl)-1H-pyrazolo[3,4-b]pyridin-1-yl)benzoic acid (INT 24C)

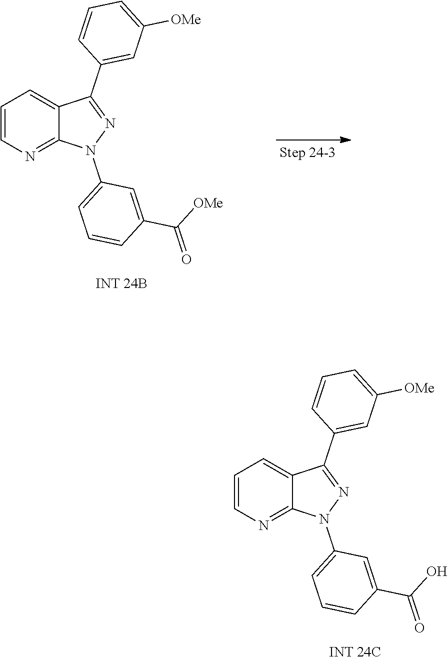 [SMILES: COC(=O)c1cccc(-n2nc(-c3cccc(OC)c3)c3cccnc32)c1 ; COc1cccc(-c2nn(-c3cccc(C(=O)O)c3)c3ncccc23)c1]

To a stirring solution of INT 24B (62 mg, 0.17 mmol) in THF (3 mL) was added 1N sodium hydroxide (0.35 mL, 1.7 mmol). The solution was stirred at 50° C. for 3 days and then was concentrated under reduced pressure. The residue was dissolved in H₂O, and the aqueous solution was acidified to pH 1 with 2N HCl and extracted with EA (3×). The organic layers were combined, dried (Na₂SO₄) and concentrated under reduced pressure to yield 55 mg (93%) of 3-(3-(3-methoxyphenyl)-1H-pyrazolo[3,4-b]pyridin-1-yl) benzoic acid (INT 24C) as a white solid. LCMS-ESI (m/z) calculated for C₂₀H₁₅N₃O₃: 345.4; found 346.2 [M+H]⁺, t_{R}=1.046 min (Method 11). ¹H NMR (400 MHz, DMSO-D₆) δ=13.32 (s, 1H), 9.07 (t, J=1.9 Hz, 1H), 8.85 (dd, J=1.5, 4.5 Hz, 1H), 8.78 (dd, J=1.6, 8.2 Hz, 1H), 8.71 (ddd, J=1.1, 2.3, 8.2 Hz, 1H), 8.00 (dt, J=1.3, 7.7 Hz, 1H), 7.80 (t, J=7.9 Hz, 1H), 7.76 (dt, J=1.2, 7.7 Hz, 1H), 7.67 (dd, J=1.5, 2.6 Hz, 1H), 7.63-7.52 (m, 2H), 7.17 (ddd, J=0.9, 2.6, 8.3 Hz, 1H), 3.96 (s, 3H).

### Step 24-3. Synthesis of N-(4-(dimethylamino)phenyl)-3-(3-(3-methoxyphenyl)-1H-pyrazolo[3,4-b]pyridin-1-yl)benzamide (Compound 24-1)

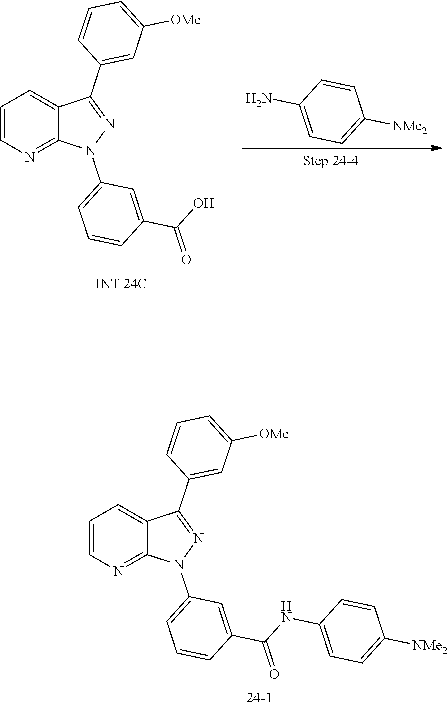 [SMILES: COc1cccc(-c2nn(-c3cccc(C(=O)O)c3)c3ncccc23)c1 ; CN(C)c1ccc(N)cc1 ; COc1cccc(-c2nn(-c3cccc(C(=O)Nc4ccc(N(C)C)cc4)c3)c3ncccc23)c1]

To a solution of INT 24C (52 mg, 0.15 mmol) and DIEA (0.11 mL, 0.60 mmol) in DMF (3 mL) was added HATU (57 mg, 0.15 mmol). After 5 min, N,N-dimethyl-p-phenylenediamine (21 mg, 0.15 mmol) was added and the resulting mixture was allowed to stir at room temperature for 17 h. The reaction mixture was passed through a syringe filter and the filtrate was purified by prep HPLC. The relevant fractions were directly lyophilized to yield 5.7 mg (8%) of N-(4-(dimethylamino)phenyl)-3-(3-(3-methoxyphenyl)-1H-pyrazolo[3,4-b]pyridin-1-yl)benzamide (Compound 24-1) as an off-white solid. LCMS-ESI (m/z) calculated for C₂₈H₂₅N₅O₂: 463.5; found 464.0 [M+H]⁺, t_{R}=8.46 min (Method 1). ¹H NMR (400 MHZ, DMSO-D₆) δ=10.12 (s, 1H), 8.81 (t, J=2.0 Hz, 1H), 8.71 (dd, J=1.6, 4.5 Hz, 1H), 8.67 (dd, J=1.6, 8.2 Hz, 1H), 8.50 (ddd, J=1.0, 2.2, 8.1 Hz, 1H), 7.88 (dt, J=1.4, 7.8 Hz, 1H), 7.72-7.61 (m, 2H), 7.59-7.51 (m, 3H), 7.55-7.43 (m, 1H), 7.46-7.39 (m, 1H), 7.04 (ddd, J=1.0, 2.7, 8.3 Hz, 1H), 6.73-6.64 (m, 2H), 3.83 (s, 3H), 2.82 (s, 6H).

The compound listed in Table 24 were made using the procedures of Scheme 24.

TABLE 24

| | | Purity | | | | |
|---|---|---|---|---|---|---|
| | Cpd | RT | | observed | | Purity |
| Structure | No. | (min) | MW | m/z | Ion | Method |
| 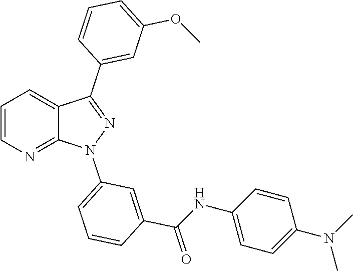 [SMILES: COc1cccc(-c2nn(-c3cccc(C(=O)Nc4ccc(N(C)C)cc4)c3)c3ncccc23)c1] | 24-1 | 8.455 | 463.2 | 464 | [M + H]⁺ | 1 |
| 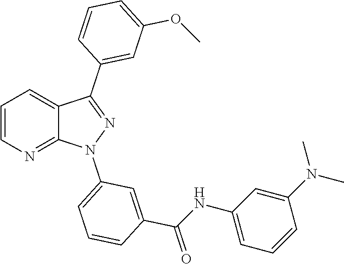 [SMILES: COc1cccc(-c2nn(-c3cccc(C(=O)Nc4cccc(N(C)C)c4)c3)c3ncccc23)c1] | 24-2 | 10.34 | 463.2 | 464 | [M + H]⁺ | 1 |

### Example 25

### Synthesis of Compound 25-1 and Other Representative Compounds

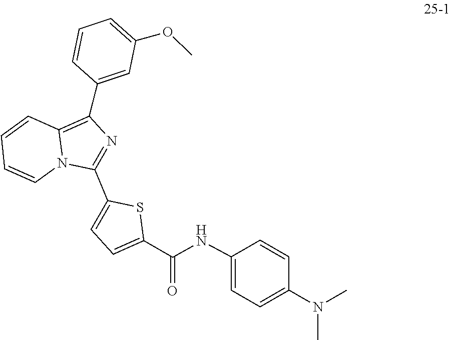 [SMILES: COc1cccc(-c2nc(-c3ccc(C(=O)Nc4ccc(N(C)C)cc4)s3)n3ccccc23)c1]

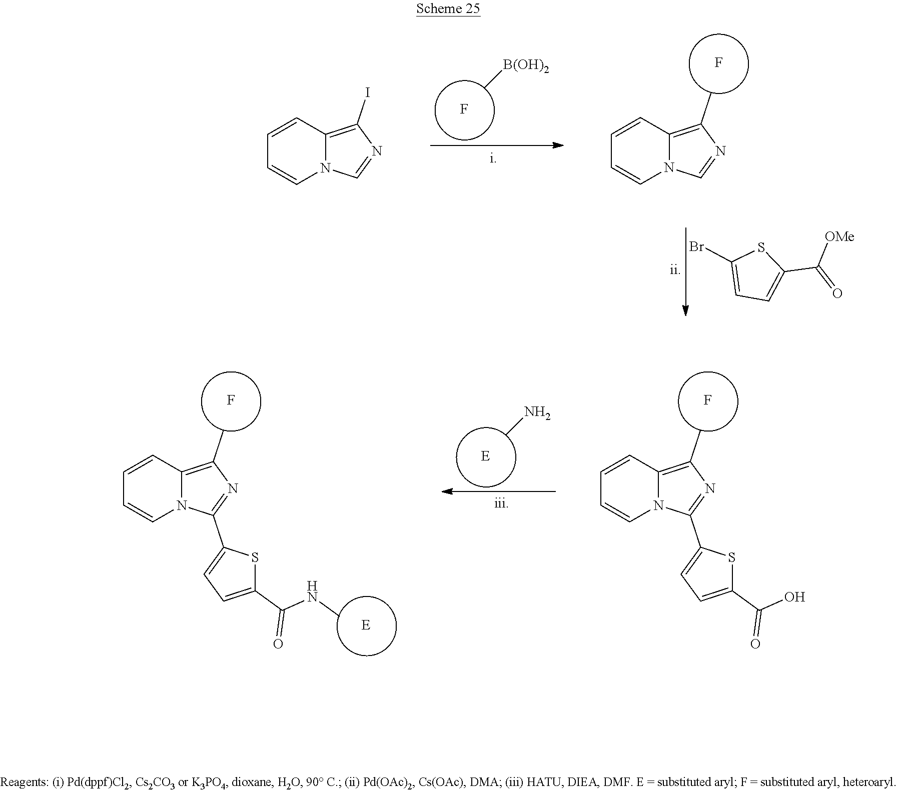

### Step 25-1. Synthesis of 1-(3-methoxyphenyl)imidazo[1,5-a]pyridine (INT 25A)

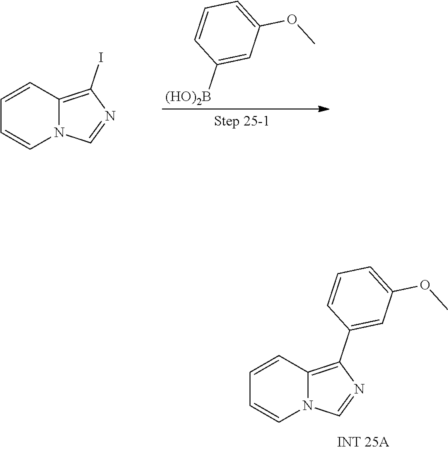 [SMILES: Ic1ncn2ccccc12 ; COc1cccc(B(O)O)c1 ; COc1cccc(-c2ncn3ccccc23)c1]

A pressure vessel containing a mixture of 1-iodoimidazo[1,5-a]pyridine (1 g, 4.10 mmol), 3-methoxyphenylboronic acid (685 mg, 4.51 mmol), [1,1′-Bis (diphenylphosphino) ferrocene]dichloropalladium(II) complex with dichloromethane (669.3 mg, 819.5 μmol), and cesium carbonate (2.67 g, 8.20 mmol) in 1,4-Dioxane (20 mL) and H₂O (2.0 mL) was degassed with N₂ gas for 15 min. The vial was capped and the mixture was heated at 90° C. for 20 h. The reaction mixture was partitioned between EA and H₂O. The organic phase was collected, and the aqueous layer was extracted with EA (2×). The organic layers were combined, washed with brine, dried (Na₂SO₄) and concentrated under reduced pressure. The residue was dissolved in DCM (12 mL) and purified by SiO₂ chromatography (EA/hexanes). The relevant fractions were combined and concentrated under reduced pressure to yield 191.8 mg (21%) of 1-(3-methoxyphenyl)imidazo[1,5-a]pyridine (INT 25A) as a yellow-brown oil. LCMS-ESI (m/z) calculated for C₁₄H₁₂N₂O: 224.3; found 225.0 [M+H]⁺, t_{R}=0.687 min (Method 11). ¹H NMR (400 MHz, DMSO-D₆) δ=8.47 (s, 1H), 8.39 (dt, J=1.2, 7.1 Hz, 1H), 7.91 (dq, J=1.0, 9.3 Hz, 1H), 7.47 (dt, J=1.3, 7.7 Hz, 1H), 7.43 (dd, J=1.5, 2.7 Hz, 1H), 7.35 (t, J=7.9 Hz, 1H), 6.91 (ddd, J=1.1, 6.4, 9.3 Hz, 1H), 6.83 (ddd, J=1.0, 2.6, 8.2 Hz, 1H), 6.72 (ddd, J=1.1, 6.4, 7.3 Hz, 1H), 3.83 (s, 3H).

### Step 25-2. Synthesis of 5-(1-(3-methoxyphenyl)imidazo[1,5-a]pyridin-3-yl)thiophene-2-carboxylic acid (INT 25B)

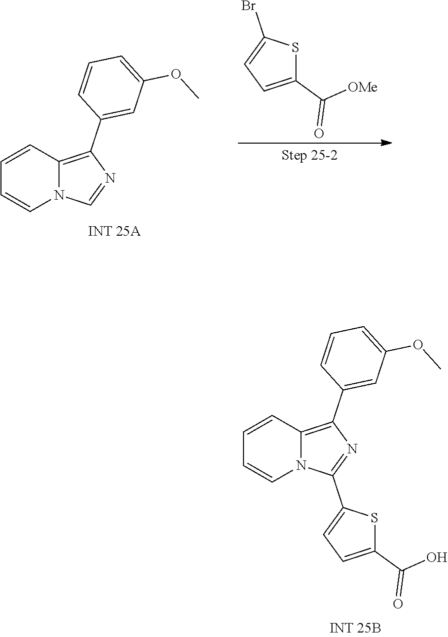 [SMILES: COc1cccc(-c2ncn3ccccc23)c1 ; COC(=O)c1ccc(Br)s1 ; COc1cccc(-c2nc(-c3ccc(C(=O)O)s3)n3ccccc23)c1]

A pressure vial containing a solution of INT 25A (186 mg, 829 μmol) and methyl 5-bromothiophene-2-carboxylate (183 mg, 829 μmol) in DMA (5 mL) was charged with cesium acetate (318 mg, 1.66 mmol) and palladium(II) acetate (9.31 mg, 41.5 μmol). The vial was capped, and the resulting mixture was stirred at 150° C. 18 h. The reaction mixture was diluted with saturated NH₄Cl and H₂O, then extracted with EA. The organic phase was collected, and the aqueous layer was extracted with EA (2×). The organic layers were combined, washed with brine, and concentrated under reduced pressure. The residue was dissolved in DCM (12 mL) and purified by SiO₂ chromatography (EA/hexanes). The relevant fractions were combined and concentrated under reduced pressure to yield 61.8 mg (21%) of 5-(1-(3-methoxyphenyl)imidazo[1,5-a]pyridin-3-yl)thiophene-2-carboxylic acid (INT 25B) as an orange solid. LCMS-ESI (m/z) calculated for C₁₉H₁₄N₂O₃S: 350.4; found 351.0 [M+H]⁺, t_{R}=0.948 min (Method 11). ¹H NMR (400 MHZ, DMSO-D₆) δ=13.29 (s, 1H), 8.72 (d, J=7.2 Hz, 1H), 8.08 (d, J=9.1 Hz, 1H), 7.83 (q, J=4.0 Hz, 2H), 7.52 (d, J=8.1 Hz, 1H), 7.45 (t, J=2.1 Hz, 1H), 7.42 (t, J=7.9 Hz, 1H), 7.10 (dd, J=6.4, 9.2 Hz, 1H), 6.99 (t, J=6.8 Hz, 1H), 6.92 (dd, J=2.6, 8.2 Hz, 1H), 3.86 (s, 3H).

### Step 25-3. Synthesis of 1-(3′-acetyl-6-(1,3,5-trimethyl-1H-pyrazol-4-yl)-[1,1′-biphenyl]-3-yl)-5-cyclopropyl-N-(pyridin-4-yl)-1H-1,2,3-triazole-4-carboxamide (Compound 25-1)

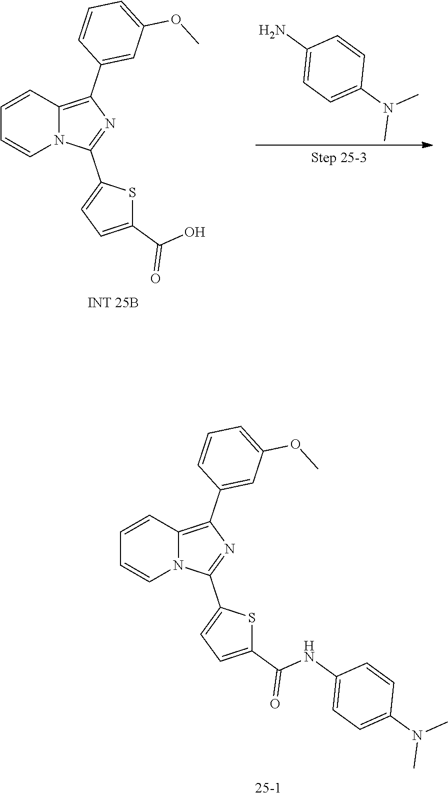

A vial containing a stirring solution of INT 25B (55 mg, 0.16 mmol) and DIEA (0.11 mL, 0.63 mmol) in DMF (3 mL) was charged with HATU (60 mg, 0.16 mmol). After 5 minutes, the reaction mixture was charged with N,N-dimethyl-p-phenylenediamine (21 mg, 0.16 mmol). The resulting mixture was stirred at room temperature for 19 h. The reaction mixture was passed through a syringe filter into a test tube, and the filtrate was directly purified by prep HPLC. The relevant fractions were lyophilized to yield 23.5 mg (32%) of N-(4-(dimethylamino) phenyl)-5-(1-(3-methoxyphenyl) imidazo[1,5-a]pyridin-3-yl)thiophene-2-carboxamide (Compound 25-1) as a yellow-orange solid. LCMS-ESI (m/z) calculated for C₂₇H₂₄N₄O₂S: 468.6; found 469.2 [M+H]⁺, t_{R}=7.952 min (Method 1). ¹H NMR (400 MHZ, DMSO) δ 10.02 (s, 1H), 8.66 (dd, J=1.2, 7.2 Hz, 1H), 8.04-7.97 (m, 2H), 7.80 (d, J=4.1 Hz, 1H), 7.52-7.46 (m, 1H), 7.50-7.42 (m, 2H), 7.38 (dd, J=1.5, 2.6 Hz, 1H), 7.34 (t, J=7.9 Hz, 1H), 7.02 (ddd, J=1.0, 6.5, 9.2 Hz, 1H), 6.92 (td, J=1.2, 6.5, 6.9 Hz, 1H), 6.85 (ddd, J=1.0, 2.6, 8.3 Hz, 1H), 6.72-6.62 (m, 2H), 3.79 (s, 3H), 2.82 (s, 6H).

The compounds listed in Table 25 were made using the procedures of Scheme 25.

TABLE 25

| | | Purity | | | | |
|---|---|---|---|---|---|---|
| | Cpd | RT | | observed | | Purity |
| Structure | No. | (min) | MW | m/z | Ion | Method |
| 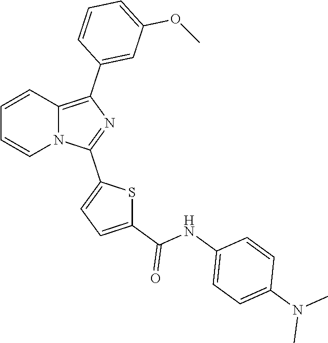 [SMILES: COc1cccc(-c2nc(-c3ccc(C(=O)Nc4ccc(N(C)C)cc4)s3)n3ccccc23)c1] | 25-1 | 7.952 | 468.2 | 469.2 | [M + H]⁺ | 1 |

### Example 26

### Synthesis of Compound 26-1 and Other Representative Compounds

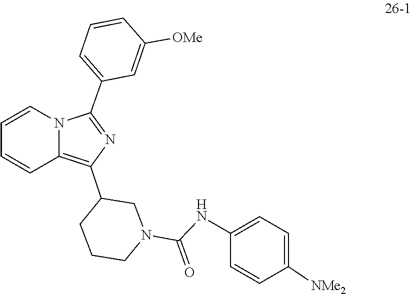 [SMILES: COc1cccc(-c2nc(C3CCCN(C(=O)Nc4ccc(N(C)C)cc4)C3)c3ccccn23)c1]

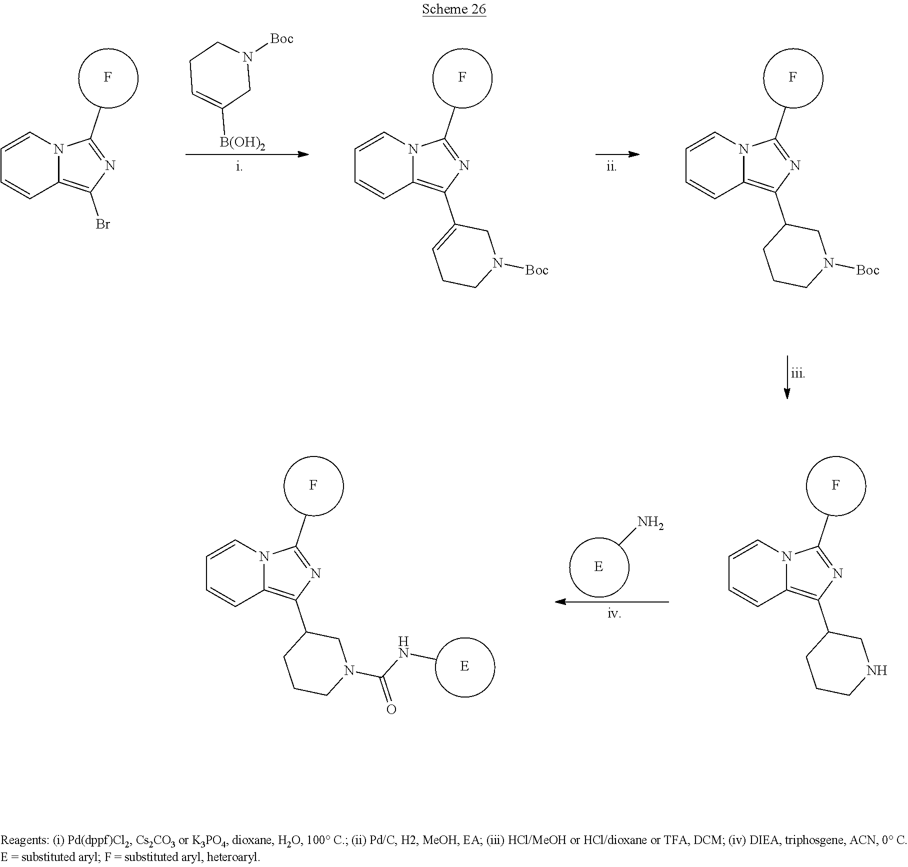

### Step 26-1. Synthesis of tert-butyl 5-(3-(3-methoxyphenyl)imidazo[1,5-a]pyridin-1-yl)-3,6-dihydropyridine-1(2H)-carboxylate (INT 26A)

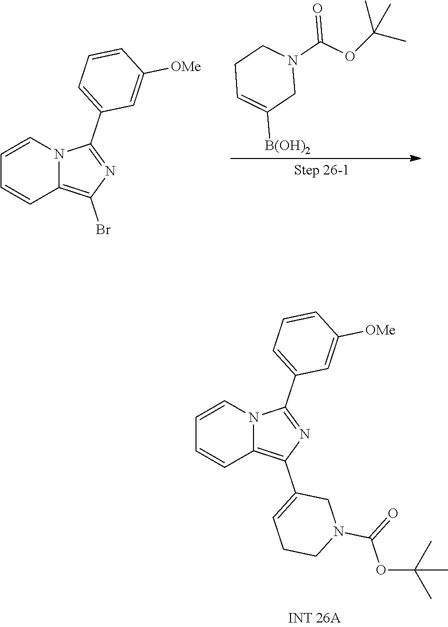 [SMILES: COc1cccc(-c2nc(Br)c3ccccn23)c1 ; CC(C)(C)OC(=O)N1CCC=C(B(O)O)C1 ; COc1cccc(-c2nc(C3=CCCN(C(=O)OC(C)(C)C)C3)c3ccccn23)c1]

To a reaction vessel containing 1-bromo-3-(3-methoxyphenyl) imidazo[1,5-a]pyridine (400 mg, 1.31 mmol) in dioxane (5 mL) and H₂O (0.5 mL) were added (1-(tert-butoxycarbonyl)-1,2,5,6-tetrahydropyridin-3-yl)boronic acid (816 mg, 2.63 mmol), [1,1′-Bis (diphenylphosphino) ferrocene]dichloropalladium(II) complex (96 mg, 131 μmol), and K₂PO₄ (840 mg, 3.96 mmol). The mixture was degassed with N₂ gas for 15 min. The vial was capped, and the mixture was heated at 100° C. for 1 h. The reaction mixture diluted with EA and washed with H₂O, saturated NaHCO₃ (aq), and brine. The organic layers were combined, dried (Na₂SO₄) and concentrated under reduced pressure then purified by SiO₂ chromatography (EA/hexanes). The relevant fractions were combined and concentrated under reduced pressure to yield 250 mg (47%) of tert-butyl 5-(3-(3-methoxyphenyl) imidazo[1,5-a]pyridin-1-yl)-3,6-dihydropyridine-1(2H)-carboxylate (INT 26A). LCMS-ESI (m/z) calculated for C₂₄H₂₇N₃O₃: 405.5; found 406.0 [M+H]⁺, t_{R}=6.41 min (Method 2).

### Step 26-2. Synthesis of tert-butyl 3-(3-(3-methoxyphenyl)imidazo[1,5-a]pyridin-1-yl)piperidine-1-carboxylate (INT 26B)

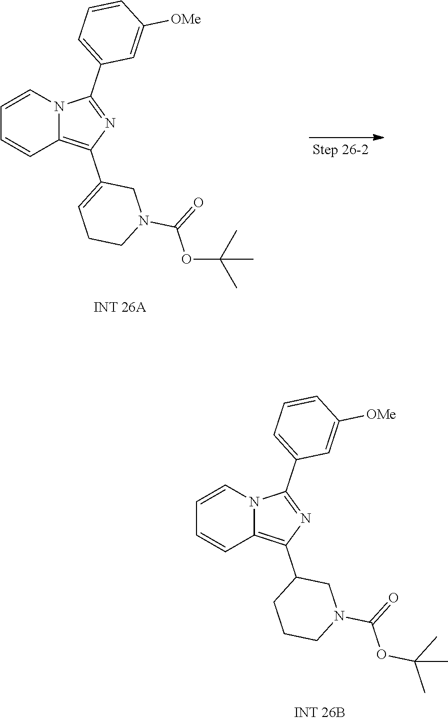 [SMILES: COc1cccc(-c2nc(C3=CCCN(C(=O)OC(C)(C)C)C3)c3ccccn23)c1 ; COc1cccc(-c2nc(C3CCCN(C(=O)OC(C)(C)C)C3)c3ccccn23)c1]

A flask containing INT 26A (100 mg, 0.246 mmol) in 1:1 MeOH/EA (10 mL) was degassed by N₂ bubbling prior to addition of 5% weight Pd/C (12.5 mg). The mixture was further degassed with N₂, then left to stir under an atmosphere of H₂, provided by a balloon filled with H₂. After 15 min, the mixture was filtered through celite and concentrated to provide 30 mg (30%) of crude tert-butyl 3-(3-(3-methoxyphenyl)imidazo[1,5-a]pyridin-1-yl)piperidine-1-carboxylate (INT 26B) as an oil that was carried forward to the next step without further purification. LCMS-ESI (m/z) calculated for C₂₄H₂₉N₃O₃: 407.5; found 408.1 [M+H]⁺, t_{R}=6.03 min (Method 2).

### Step 26-3. Synthesis of 3-(3-methoxyphenyl)-1-(piperidin-3-yl)imidazo[1,5-a]pyridine (INT 26C)

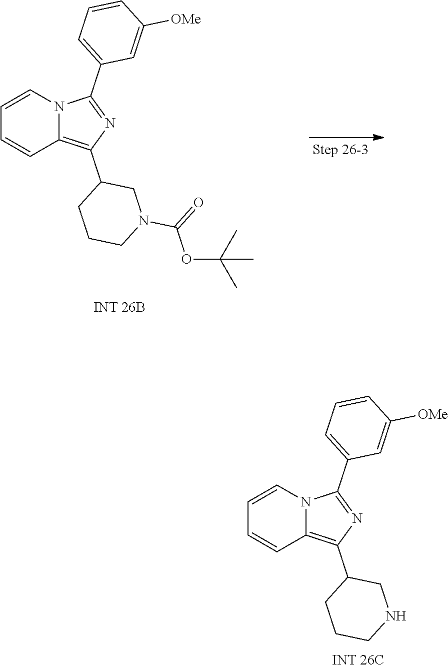 [SMILES: COc1cccc(-c2nc(C3CCCN(C(=O)OC(C)(C)C)C3)c3ccccn23)c1 ; COc1cccc(-c2nc(C3CCCNC3)c3ccccn23)c1]

To a vial containing a stirring solution of INT 26B (30 mg, 0.07 mmol) in DCM (4 mL) was added 4N HCl/dioxane (2 mL). After 3 h, the reaction mixture was concentrated to yield 25 mg (100%) of 3-(3-methoxyphenyl)-1-(piperidin-3-yl) imidazo[1,5-a]pyridine (INT 26C) that was used in the next step without further purification.

### Step 26-4. Synthesis of N-(4-(dimethylamino)phenyl)-3-(3-(3-methoxyphenyl) imidazo[1,5-a]pyridin-1-yl)piperidine-1-carboxamide (Compound 26-1)

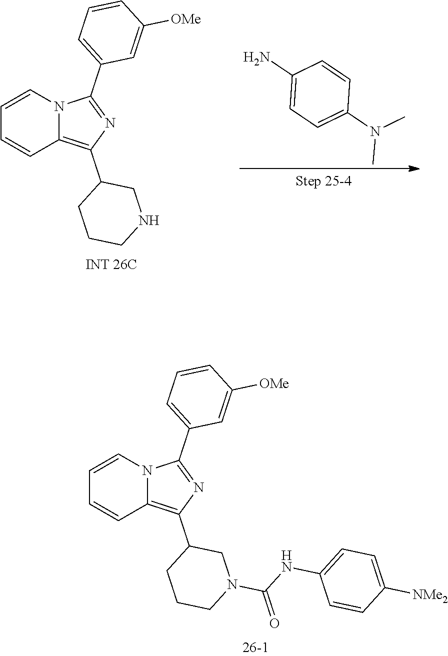

To a mixture of N1,N1-dimethylbenzene-1,4-diamine (12 mg, 0.08 mmol) and triphosgene (9.9 mg, 0.03 mmol) in DCM (5 mL) was added DIEA (114 mg, 0.88 mmol) at 0° C. After stirring at 0° C. for 10 min, INT 26C (27 mg, 0.088 mmol) was added. After stirring at rt for 1 h, the mixture was quenched with H₂O (15 mL) and extracted with DCM (3×). The combined organic layers were dried (Na₂SO₄), concentrated, and purified by SiO₂ chromatography (EA/hexane) and reversed phase chromatography (MeOH/H₂O) to afford 12 mg (29%) of N-(4-(dimethylamino)phenyl)-3-(3-(3-methoxyphenyl) imidazo[1,5-a]pyridin-1-yl)piperidine-1-carboxamide (Compound 26-1). LCMS-ESI (m/z) calculated for C₂₈H₃₁N₅O₂: 469.6; found 470.3 [M+H]⁺, t_{R}=9.21 min (Method 2).

The compounds listed in Table 26 were made using the procedures of Scheme 26.

TABLE 26

| | | Purity | | | | |
|---|---|---|---|---|---|---|
| | Cpd | RT | | observed | | Purity |
| Structure | No. | (min) | MW | m/z | Ion | Method |
| 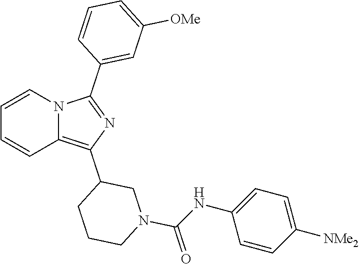 [SMILES: COc1cccc(-c2nc(C3CCCN(C(=O)Nc4ccc(N(C)C)cc4)C3)c3ccccn23)c1] | 26-1 | 9.15 | 469.2 | 470.3 | [M + H]⁺ | 2 |

### Example 27

### MRGPRD Activity

Chinese Hamster Ovary (CHO) cells stably expressing human MRGPRD (Eurofins, San Diego CA) were plated in a 384-well plate at 20,000 cells per well in 12 μL of Optimem (Fisher Scientific 11-058-021). Plates were kept in the incubator at 37° C. overnight. Antagonists were solubilized at a concentration of 10 mM in DMSO, and the agonists were solubilized to stock concentrations of either 50 mM in H₂O (β-Alanine) or 10 mM in DMSO (all others) and then diluted in assay buffer (final concentrations of 5.7 mM Tris-HCl, 43 mM NaCl, 50 mM LiCl, pH=8). The concentration of DMSO was normalized across the plate. Antagonists were added to the plate using a Tecan D300 Digital Dispenser at nanoliter/picoliter volumes and 2 μL of agonists in assay buffer were added to each well for a final assay volume of 14 μL. Plates were covered and incubated for 1 h at 37° C. and then for 1 h at room temperature. IP-1 standards and HTRF detection reagents were added according to an IP-One-Gq Kit purchased from Cisbio (part number 62IPAPEJ) and incubated in the dark for 1 h at room temperature. Assay plates were read on either a Molecular Devices SpectraMax iD5 plate reader or a BMG ClarioStar plate reader. The HTRF ratio was calculated form the raw data and graphed using GraphPad Prism to calculate IC₅₀ values. Data are expressed as average IC₅₀ values and average percent antagonism calculated as a percent of the maximum efficacy response. For these studies, the variability of the data was assessed by determining the standard error of mean (SEM).

Activity data for selected MRGPRD antagonists (versus 100 μM β-Alanine agonist) are displayed in Table 27. The activity ranges are denoted as follows: “+++++” denotes antagonist activity <100 nM; “++++” denotes antagonist activity between 100 and 500 nM; “+++” denotes activity between 500 and 1000 nM; “++” denotes activity between 1000 and 3000 nM; and “+” denotes activity >3000 nM.

TABLE 27
Activity data for selected MRGPRD antagonists

| Cpd No. | mRGPRD Antagonist Activity |
|---|---|
| 1-1 | ++++ |
| 1-2 | +++++ |
| 1-3 | ++ |
| 1-4 | +++ |
| 1-5 | ++ |
| 1-6 | +++++ |
| 1-7 | +++++ |
| 1-8 | +++++ |
| 1-9 | +++++ |
| 1-10 | ++++ |
| 1-11 | ++++ |
| 1-12 | +++++ |
| 1-13 | ++++ |
| 1-14 | ++++ |
| 1-15 | ++++ |
| 1-16 | ++++ |
| 1-17 | +++++ |
| 1-18 | ++++ |
| 1-19 | +++++ |
| 1-20 | +++++ |
| 1-21 | +++ |
| 1-22 | +++ |
| 1-23 | +++ |
| 1-24 | +++++ |
| 1-25 | +++++ |
| 1-26 | ++++ |
| 1-27 | +++ |
| 1-28 | ++++ |
| 1-29 | +++ |
| 1-30 | ++++ |
| 1-31 | ++++ |
| 1-32 | +++++ |
| 1-33 | +++++ |
| 2-1 | + |
| 2-2 | + |
| 3-1 | ++++ |
| 3-2 | ++++ |
| 3-3 | ++++ |
| 3-4 | ++ |
| 3-5 | ++++ |
| 3-6 | ++++ |
| 3-7 | +++ |
| 3-8 | ++++ |
| 3-9 | ++++ |
| 3-10 | +++ |
| 3-11 | + |
| 3-12 | ++++ |
| 3-13 | + |
| 3-14 | + |
| 3-15 | + |
| 3-16 | ++++ |
| 3-17 | +++ |
| 3-18 | + |
| 3-19 | ++++ |
| 3-20 | +++ |
| 3-21 | ++++ |
| 3-22 | ++++ |
| 3-23 | ++++ |
| 3-24 | ++++ |
| 3-25 | +++++ |
| 3-26 | ++++ |
| 3-27 | +++++ |
| 3-28 | ++++ |
| 3-29 | ++ |
| 4-1 | ++++ |
| 4-2 | +++++ |
| 4-3 | +++ |
| 4-4 | +++ |
| 5-1 | ++ |
| 5-2 | ++ |
| 5-3 | +++ |
| 5-4 | ++++ |
| 5-5 | ++++ |
| 5-6 | +++++ |
| 5-7 | +++++ |
| 5-8 | ++ |
| 5-9 | + |
| 5-10 | ++++ |
| 5-11 | ++++ |
| 5-12 | +++++ |
| 5-13 | +++ |
| 5-14 | ++++ |
| 5-15 | +++++ |
| 5-16 | +++++ |
| 5-17 | +++++ |
| 5-18 | +++++ |
| 5-19 | +++++ |
| 5-20 | +++++ |
| 5-21 | +++ |
| 5-22 | ++ |
| 5-23 | +++ |
| 5-24 | +++++ |
| 5-25 | +++++ |
| 5-26 | ++++ |
| 5-27 | +++++ |
| 5-28 | +++++ |
| 5-29 | ++ |
| 5-30 | ++++ |
| 5-31 | ++++ |
| 5-32 | ++++ |
| 5-33 | ++ |
| 5-34 | ++ |
| 5-35 | +++++ |
| 5-36 | ++++ |
| 5-37 | ++++ |
| 5-38 | ++++ |
| 5-39 | ++++ |
| 5-40 | +++++ |
| 5-41 | ++++ |
| 5-42 | +++++ |
| 5-43 | ++++ |
| 5-44 | +++++ |
| 5-45 | +++++ |
| 5-46 | +++ |
| 5-47 | +++++ |
| 5-48 | ++++ |
| 5-49 | +++++ |
| 5-50 | +++++ |
| 5-51 | +++++ |
| 5-52 | ++++ |
| 5-53 | +++++ |
| 5-54 | +++ |
| 5-55 | +++++ |
| 5-56 | ++++ |
| 5-57 | +++++ |
| 5-58 | +++++ |
| 5-59 | +++++ |
| 5-60 | +++++ |
| 5-61 | +++++ |
| 5-62 | +++++ |
| 5-63 | +++++ |
| 5-64 | +++ |
| 5-65 | +++++ |
| 5-66 | +++++ |
| 5-67 | +++++ |
| 5-68 | +++++ |
| 5-69 | +++++ |
| 5-70 | +++++ |
| 5-71 | +++++ |
| 5-72 | +++++ |
| 5-73 | +++++ |
| 6-1 | +++++ |
| 7-1 | +++++ |
| 8-1 | ++++ |
| 8-2 | +++++ |
| 9-1 | +++++ |
| 9-2 | +++++ |
| 10-1 | ++++ |
| 10-2 | +++ |
| 10-3 | +++ |
| 10-4 | +++++ |
| 10-5 | +++ |
| 10-6 | ++++ |
| 10-7 | +++++ |
| 10-8 | +++ |
| 10-9 | +++ |
| 10-10 | + |
| 10-11 | +++ |
| 10-12 | +++ |
| 10-13 | +++ |
| 10-14 | ++ |
| 10-15 | ++ |
| 10-16 | ++++ |
| 10-17 | +++++ |
| 10-18 | ++++ |
| 10-19 | +++ |
| 10-20 | + |
| 10-21 | ++++ |
| 10-22 | +++ |
| 10-23 | +++ |
| 10-24 | +++ |
| 10-25 | ++++ |
| 10-26 | +++ |
| 10-27 | +++ |
| 10-28 | ++ |
| 10-29 | +++ |
| 10-30 | ++ |
| 10-31 | + |
| 10-32 | ++ |
| 10-33 | + |
| 10-34 | ++ |
| 10-35 | + |
| 10-36 | +++ |
| 10-37 | +++ |
| 10-38 | +++ |
| 10-39 | + |
| 10-40 | +++ |
| 10-41 | ++ |
| 10-42 | +++ |
| 10-43 | +++ |
| 10-44 | ++++ |
| 10-45 | ++++ |
| 10-46 | + |
| 10-47 | ++++ |
| 10-48 | ++ |
| 10-49 | ++++ |
| 10-50 | +++++ |
| 10-51 | +++++ |
| 10-52 | ++ |
| 10-53 | +++ |
| 10-54 | +++ |
| 10-55 | +++ |
| 10-56 | +++ |
| 10-57 | +++ |
| 10-58 | +++ |
| 10-59 | ++ |
| 10-60 | + |
| 10-61 | + |
| 10-62 | ++ |
| 10-63 | ++ |
| 10-64 | + |
| 10-65 | +++ |
| 10-66 | + |
| 10-67 | ++ |
| 10-68 | +++ |
| 10-69 | +++ |
| 10-70 | +++++ |
| 10-71 | +++++ |
| 10-72 | ++++ |
| 11-1 | ++ |
| 11-2 | ++ |
| 11-3 | ++ |
| 11-4 | + |
| 11-5 | ++ |
| 11-6 | + |
| 11-7 | ++ |
| 11-8 | ++ |
| 11-9 | ++ |
| 11-10 | ++ |
| 11-11 | + |
| 12-1 | ++++ |
| 12-2 | +++ |
| 12-3 | +++ |
| 12-4 | ++++ |
| 12-5 | +++ |
| 12-6 | ++++ |
| 12-7 | +++ |
| 12-8 | ++ |
| 12-9 | +++ |
| 12-10 | ++ |
| 12-11 | +++ |
| 12-12 | +++ |
| 12-13 | ++++ |
| 12-14 | +++ |
| 12-15 | ++++ |
| 12-16 | ++ |
| 13-1 | ++++ |
| 14-1 | +++ |
| 14-2 | + |
| 14-3 | + |
| 14-4 | ++++ |
| 14-5 | ++++ |
| 15-1 | +++ |
| 16-1 | +++ |
| 16-2 | ++++ |
| 16-3 | + |
| 16-4 | ++++ |
| 16-5 | + |
| 16-6 | ++++ |
| 16-7 | ++++ |
| 16-8 | ++ |
| 16-9 | +++++ |
| 16-10 | ++ |
| 17-1 | ++++ |
| 17-2 | +++ |
| 17-3 | ++++ |
| 18-1 | ++++ |
| 19-1 | + |
| 19-2 | + |
| 19-3 | ++ |
| 20-1 | +++++ |
| 20-2 | +++++ |
| 20-3 | ++ |
| 20-4 | ++ |
| 21-1 | +++ |
| 21-2 | ++ |
| 21-3 | + |
| 21-4 | + |
| 21-5 | + |
| 21-6 | + |
| 22-1 | ++ |
| 23-1 | + |
| 23-2 | + |
| 24-1 | ++++ |
| 24-2 | +++ |
| 25-1 | ++ |
| 26-1 | +++++ |

All of the U.S. patents, U.S. patent application publications, U.S. patent applications, foreign patents, foreign patent applications and non-patent publications referred to in this specification and/or listed in the Application Data Sheet are incorporated herein by reference, in their entirety. In addition, the terms used in the following claims should not be construed as limited to the specific embodiments disclosed in the specification but should be construed to include all possible embodiments along with the full scope of equivalents to which such claims are entitled.

This application claims the benefit of priority to U.S. Provisional Application No. 63/420,470, filed Oct. 28, 2022, which application is hereby incorporated by reference in its entirety.

1. A compound having the following structure (I): 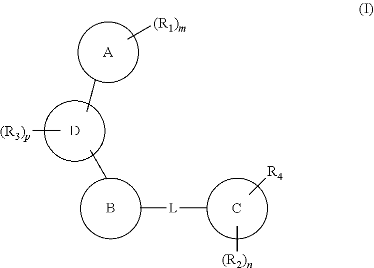
or a pharmaceutically acceptable salt, isomer, hydrate, solvate, or isotope thereof, wherein:
A and B are each individually carbocyclyl or heterocyclyl;
C is phenyl or C is optionally absent when R₄ is aminylalkyl;
D is 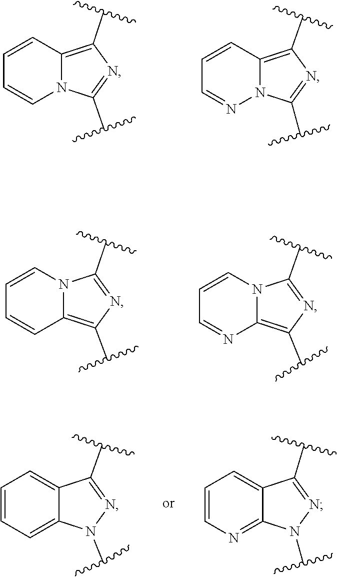
L is a linker having the structure —C(O)—, —C(O)-alkyl-, —C(O)—NRa-, —C(O)—NRa-alkyl-, —S(O)₂—, or —S(O)₂-alkyl-;
R₁ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, alkoxycarbonyl, aminylcarbonyl, alkylsulfonyl, cycloalkylalkyl, aryl, aralkyl, or arylsulfonyl;
R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
R₄ is aminyl, aminylalkyl, or aminylcarbonyl;
Ra is H or alkyl;
m is 0, 1, or 2;
n is 0, 1, 2, or 3; and
p is 0, 1, 2, or 3.

2. The compound of claim 1, wherein A is carbocyclyl.

3. The compound of claim 1, wherein A is heterocyclyl.

4-5. (canceled)

6. The compound of claim 1, wherein B is carbocyclyl.

7. (canceled)

8. The compound of claim 1, wherein B is heterocyclyl.

9-60. (canceled)

61. The compound of claim 1, having one of the following structures:

| Cpd. | |
|---|---|
| No. | Structure |
| 1-1 | 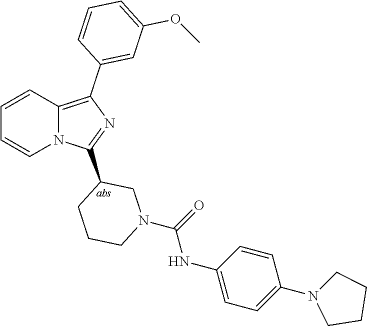 [SMILES: COc1cccc(-c2nc([C@@H]3CCCN(C(=O)Nc4ccc(N5CCCC5)cc4)C3)n3ccccc23)c1] |
| 1-2 | 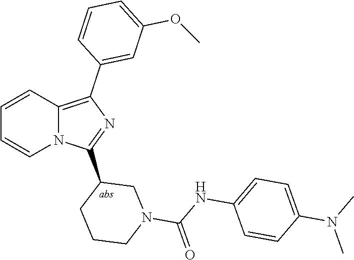 [SMILES: COc1cccc(-c2nc([C@@H]3CCCN(C(=O)Nc4ccc(N(C)C)cc4)C3)n3ccccc23)c1] |
| 1-5 | 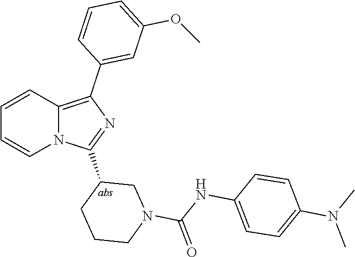 [SMILES: COc1cccc(-c2nc([C@H]3CCCN(C(=O)Nc4ccc(N(C)C)cc4)C3)n3ccccc23)c1] |
| 1-6 | 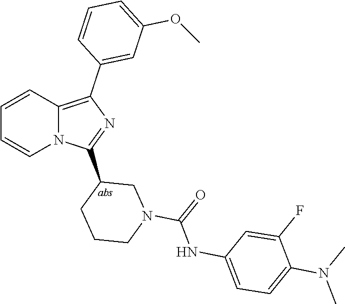 [SMILES: COc1cccc(-c2nc([C@@H]3CCCN(C(=O)Nc4ccc(N(C)C)c(F)c4)C3)n3ccccc23)c1] |
| 1-7 | 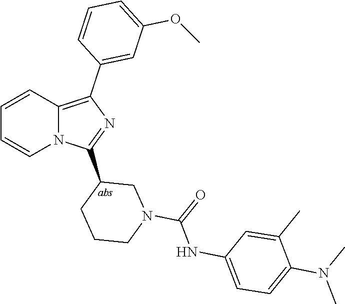 [SMILES: COc1cccc(-c2nc([C@@H]3CCCN(C(=O)Nc4ccc(N(C)C)c(C)c4)C3)n3ccccc23)c1] |
| 1-8 | 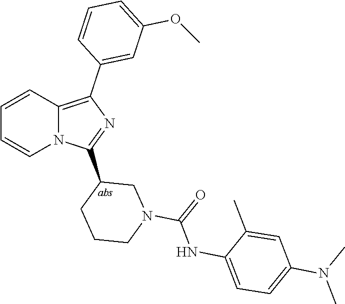 [SMILES: COc1cccc(-c2nc([C@@H]3CCCN(C(=O)Nc4ccc(N(C)C)cc4C)C3)n3ccccc23)c1] |
| 1-19 | 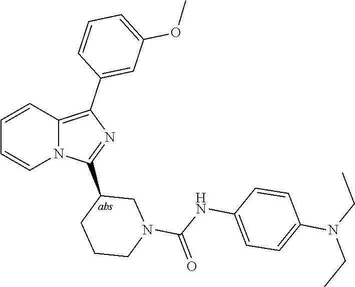 [SMILES: CCN(CC)c1ccc(NC(=O)N2CCC[C@@H](c3nc(-c4cccc(OC)c4)c4ccccn34)C2)cc1] |
| 1-20 | 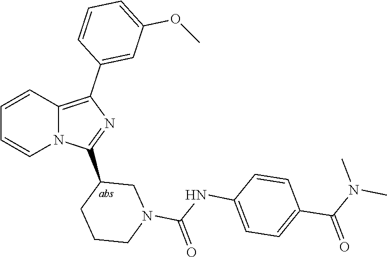 [SMILES: COc1cccc(-c2nc([C@@H]3CCCN(C(=O)Nc4ccc(C(=O)N(C)C)cc4)C3)n3ccccc23)c1] |
| 1-21 | 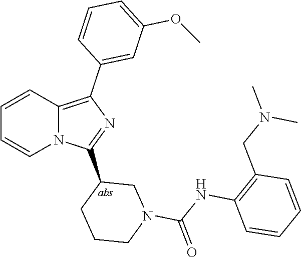 [SMILES: COc1cccc(-c2nc([C@@H]3CCCN(C(=O)Nc4ccccc4CN(C)C)C3)n3ccccc23)c1] |
| I-26 | 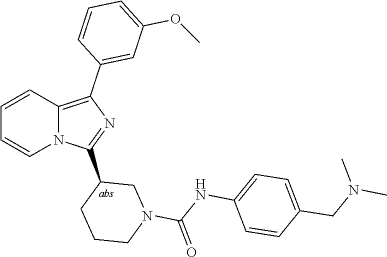 [SMILES: COc1cccc(-c2nc([C@@H]3CCCN(C(=O)Nc4ccc(CN(C)C)cc4)C3)n3ccccc23)c1] |
| I-27 | 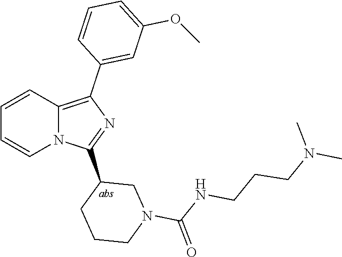 [SMILES: COc1cccc(-c2nc([C@@H]3CCCN(C(=O)NCCCN(C)C)C3)n3ccccc23)c1] |
| I-29 | 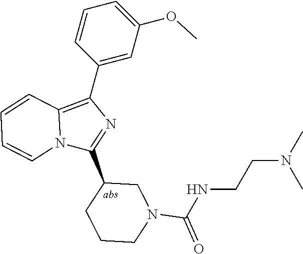 [SMILES: COc1cccc(-c2nc([C@@H]3CCCN(C(=O)NCCN(C)C)C3)n3ccccc23)c1] |
| 3-24 | 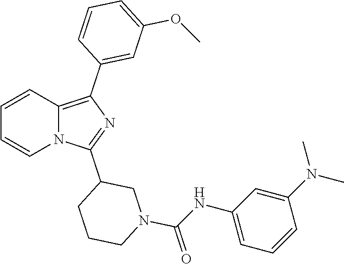 [SMILES: COc1cccc(-c2nc(C3CCCN(C(=O)Nc4cccc(N(C)C)c4)C3)n3ccccc23)c1] |
| 3-25 | 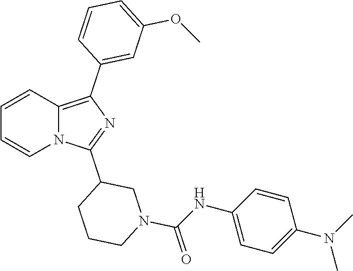 [SMILES: COc1cccc(-c2nc(C3CCCN(C(=O)Nc4ccc(N(C)C)cc4)C3)n3ccccc23)c1] |
| 4-3 | 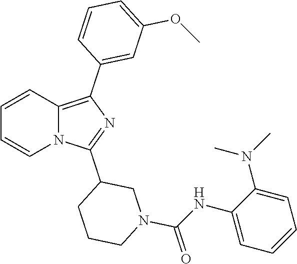 [SMILES: COc1cccc(-c2nc(C3CCCN(C(=O)Nc4ccccc4N(C)C)C3)n3ccccc23)c1] |
| 5-4 | 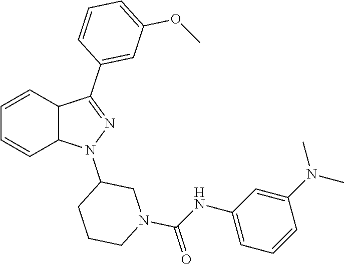 [SMILES: COc1cccc(C2=NN(C3CCCN(C(=O)Nc4cccc(N(C)C)c4)C3)C3C=CC=CC23)c1] |
| 5-6 | 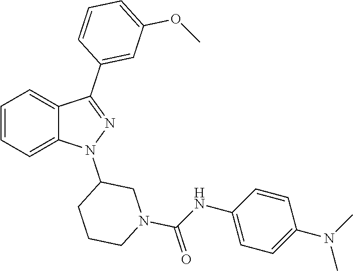 [SMILES: COc1cccc(-c2nn(C3CCCN(C(=O)Nc4ccc(N(C)C)cc4)C3)c3ccccc23)c1] |
| 5-8 | 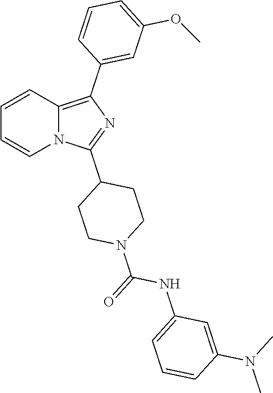 [SMILES: COc1cccc(-c2nc(C3CCN(C(=O)Nc4cccc(N(C)C)c4)CC3)n3ccccc23)c1] |
| 5-9 | 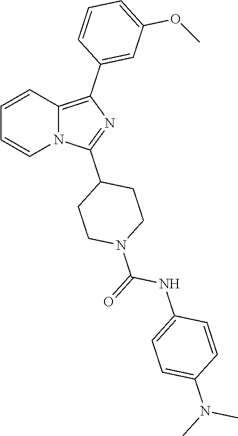 [SMILES: COc1cccc(-c2nc(C3CCN(C(=O)Nc4ccc(N(C)C)cc4)CC3)n3ccccc23)c1] |
| 5-17 | 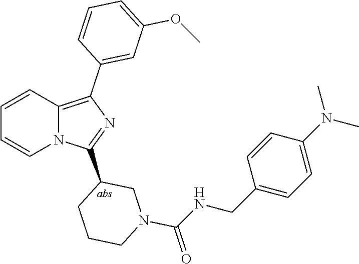 [SMILES: COc1cccc(-c2nc([C@@H]3CCCN(C(=O)NCc4ccc(N(C)C)cc4)C3)n3ccccc23)c1] |
| 5-18 | 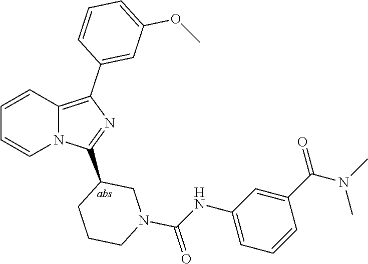 [SMILES: COc1cccc(-c2nc([C@@H]3CCCN(C(=O)Nc4cccc(C(=O)N(C)C)c4)C3)n3ccccc23)c1] |
| 5-19 | 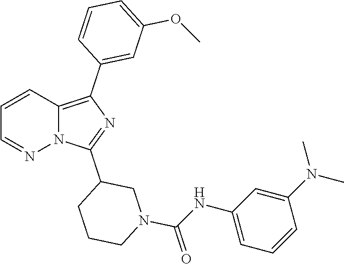 [SMILES: COc1cccc(-c2nc(C3CCCN(C(=O)Nc4cccc(N(C)C)c4)C3)n3ncccc23)c1] |
| 5-20 | 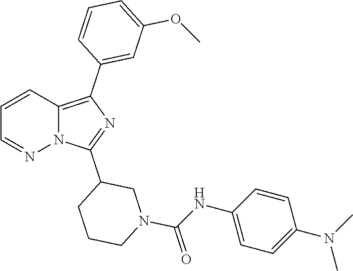 [SMILES: COc1cccc(-c2nc(C3CCCN(C(=O)Nc4ccc(N(C)C)cc4)C3)n3ncccc23)c1] |
| 5-31 | 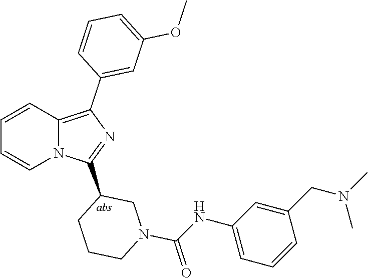 [SMILES: COc1cccc(-c2nc([C@@H]3CCCN(C(=O)Nc4cccc(CN(C)C)c4)C3)n3ccccc23)c1] |
| 5-33 | 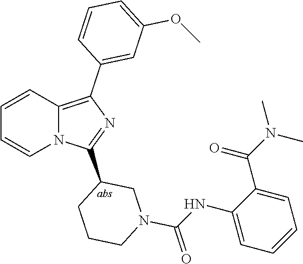 [SMILES: COc1cccc(-c2nc([C@@H]3CCCN(C(=O)Nc4ccccc4C(=O)N(C)C)C3)n3ccccc23)c1] |
| 5-36 | 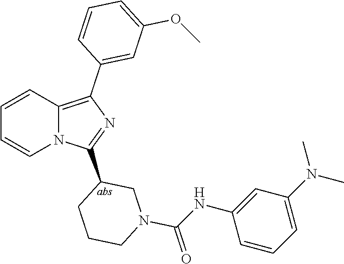 [SMILES: COc1cccc(-c2nc([C@@H]3CCCN(C(=O)Nc4cccc(N(C)C)c4)C3)n3ccccc23)c1] |
| 5-37 | 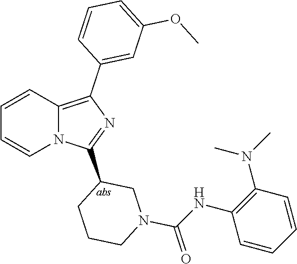 [SMILES: COc1cccc(-c2nc([C@@H]3CCCN(C(=O)Nc4ccccc4N(C)C)C3)n3ccccc23)c1] |
| 7-1 | 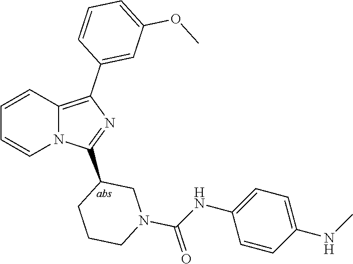 [SMILES: CNc1ccc(NC(=O)N2CCC[C@@H](c3nc(-c4cccc(OC)c4)c4ccccn34)C2)cc1] |
| 8-1 | 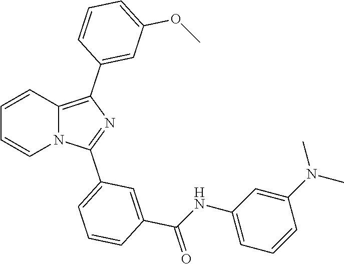 [SMILES: COc1cccc(-c2nc(-c3cccc(C(=O)Nc4cccc(N(C)C)c4)c3)n3ccccc23)c1] |
| 8-2 | 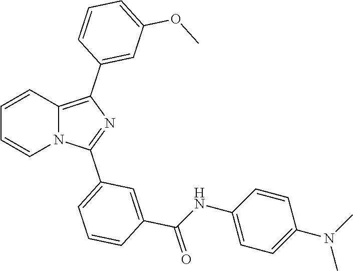 [SMILES: COc1cccc(-c2nc(-c3cccc(C(=O)Nc4ccc(N(C)C)cc4)c3)n3ccccc23)c1] |
| 9-1 | 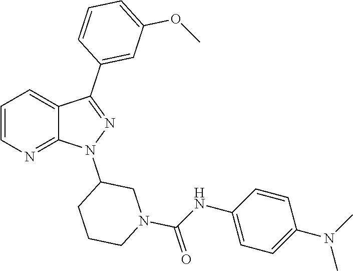 [SMILES: COc1cccc(-c2nn(C3CCCN(C(=O)Nc4ccc(N(C)C)cc4)C3)c3ncccc23)c1] |
| 9-2 | 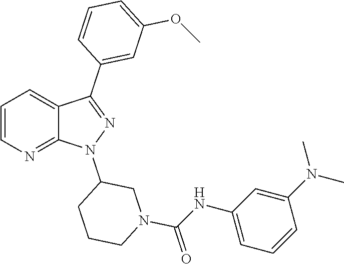 [SMILES: COc1cccc(-c2nn(C3CCCN(C(=O)Nc4cccc(N(C)C)c4)C3)c3ncccc23)c1] |
| 10-1 | 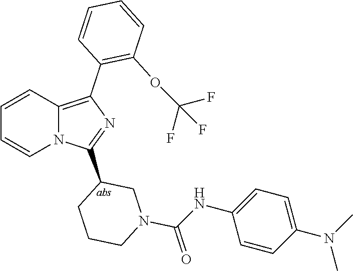 [SMILES: CN(C)c1ccc(NC(=O)N2CCC[C@@H](c3nc(-c4ccccc4OC(F)(F)F)c4ccccn34)C2)cc1] |
| 10-2 | 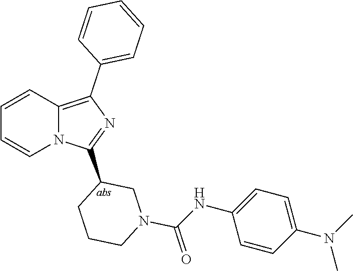 [SMILES: CN(C)c1ccc(NC(=O)N2CCC[C@@H](c3nc(-c4ccccc4)c4ccccn34)C2)cc1] |
| 10-3 | 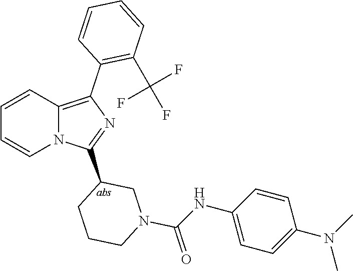 [SMILES: CN(C)c1ccc(NC(=O)N2CCC[C@@H](c3nc(-c4ccccc4C(F)(F)F)c4ccccn34)C2)cc1] |
| 10-4 | 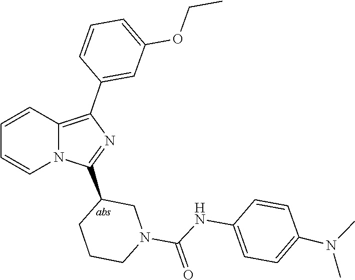 [SMILES: CCOc1cccc(-c2nc([C@@H]3CCCN(C(=O)Nc4ccc(N(C)C)cc4)C3)n3ccccc23)c1] |
| 10-5 | 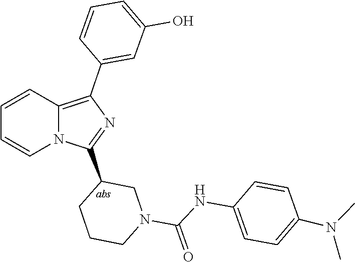 [SMILES: CN(C)c1ccc(NC(=O)N2CCC[C@@H](c3nc(-c4cccc(O)c4)c4ccccn34)C2)cc1] |
| 10-6 | 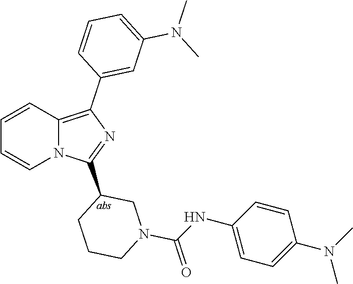 [SMILES: CN(C)c1ccc(NC(=O)N2CCC[C@@H](c3nc(-c4cccc(N(C)C)c4)c4ccccn34)C2)cc1] |
| 10-7 | 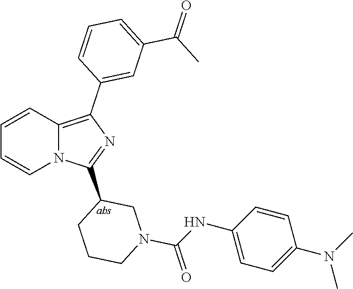 [SMILES: CC(=O)c1cccc(-c2nc([C@@H]3CCCN(C(=O)Nc4ccc(N(C)C)cc4)C3)n3ccccc23)c1] |
| 10-8 | 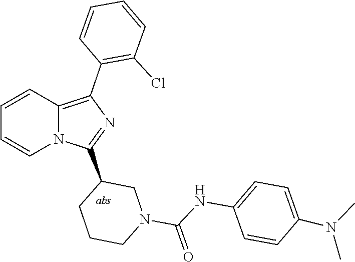 [SMILES: CN(C)c1ccc(NC(=O)N2CCC[C@@H](c3nc(-c4ccccc4Cl)c4ccccn34)C2)cc1] |
| 10-9 | 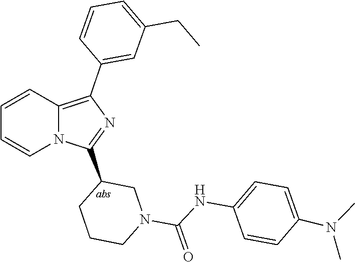 [SMILES: CCc1cccc(-c2nc([C@@H]3CCCN(C(=O)Nc4ccc(N(C)C)cc4)C3)n3ccccc23)c1] |
| 10-10 | 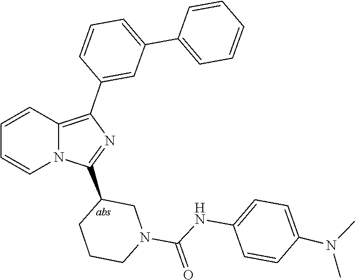 [SMILES: CN(C)c1ccc(NC(=O)N2CCC[C@@H](c3nc(-c4cccc(-c5ccccc5)c4)c4ccccn34)C2)cc1] |
| 10-11 | 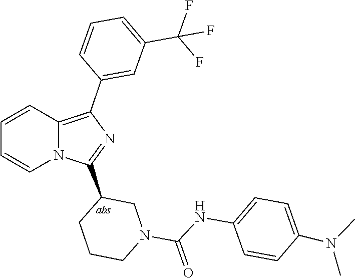 [SMILES: CN(C)c1ccc(NC(=O)N2CCC[C@@H](c3nc(-c4cccc(C(F)(F)F)c4)c4ccccn34)C2)cc1] |
| 10-12 | 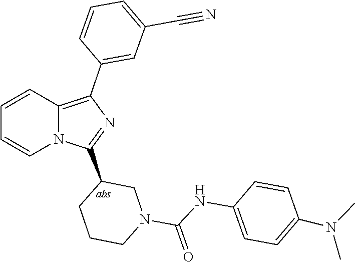 [SMILES: CN(C)c1ccc(NC(=O)N2CCC[C@@H](c3nc(-c4cccc(C#N)c4)c4ccccn34)C2)cc1] |
| 10-13 | 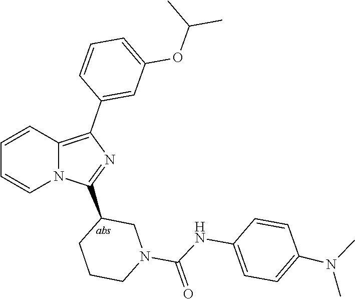 [SMILES: CC(C)Oc1cccc(-c2nc([C@@H]3CCCN(C(=O)Nc4ccc(N(C)C)cc4)C3)n3ccccc23)c1] |
| 10-14 | 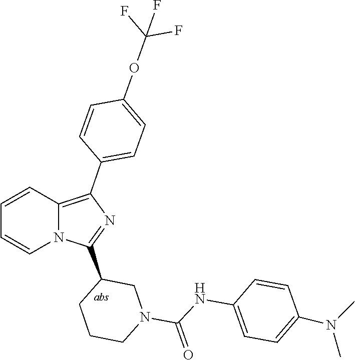 [SMILES: CN(C)c1ccc(NC(=O)N2CCC[C@@H](c3nc(-c4ccc(OC(F)(F)F)cc4)c4ccccn34)C2)cc1] |
| 10-15 | 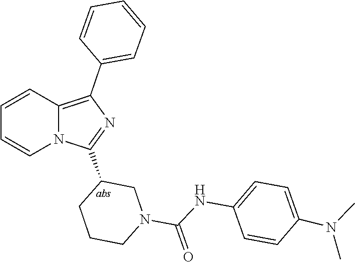 [SMILES: CN(C)c1ccc(NC(=O)N2CCC[C@H](c3nc(-c4ccccc4)c4ccccn34)C2)cc1] |
| 10-16 | 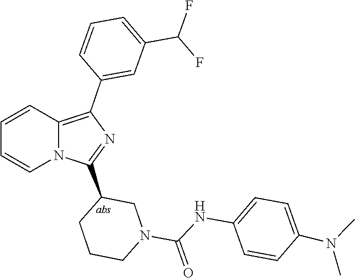 [SMILES: CN(C)c1ccc(NC(=O)N2CCC[C@@H](c3nc(-c4cccc(C(F)F)c4)c4ccccn34)C2)cc1] |
| 10-17 | 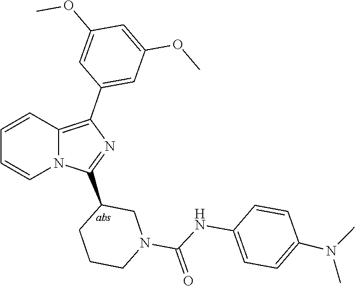 [SMILES: COc1cc(OC)cc(-c2nc([C@@H]3CCCN(C(=O)Nc4ccc(N(C)C)cc4)C3)n3ccccc23)c1] |
| 10-18 | 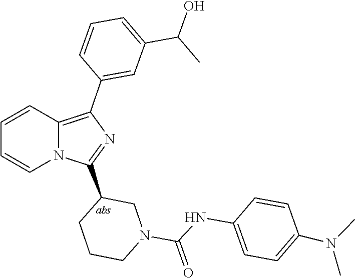 [SMILES: CC(O)c1cccc(-c2nc([C@@H]3CCCN(C(=O)Nc4ccc(N(C)C)cc4)C3)n3ccccc23)c1] |
| 10-19 | 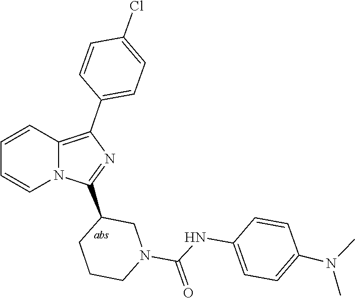 [SMILES: CN(C)c1ccc(NC(=O)N2CCC[C@@H](c3nc(-c4ccc(Cl)cc4)c4ccccn34)C2)cc1] |
| 10-20 | 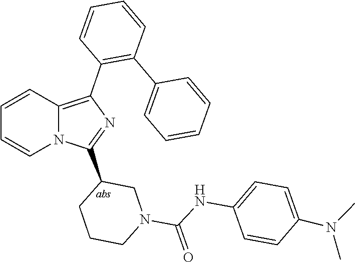 [SMILES: CN(C)c1ccc(NC(=O)N2CCC[C@@H](c3nc(-c4ccccc4-c4ccccc4)c4ccccn34)C2)cc1] |
| 10-21 | 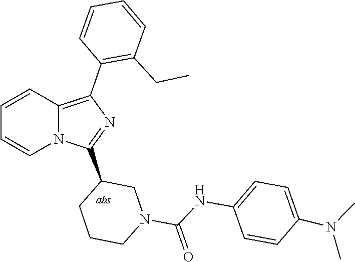 [SMILES: CCc1ccccc1-c1nc([C@@H]2CCCN(C(=O)Nc3ccc(N(C)C)cc3)C2)n2ccccc12] |
| 10-22 | 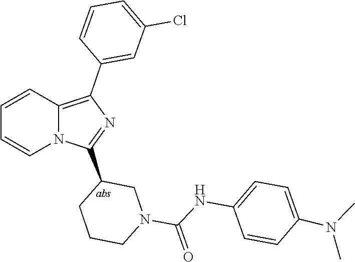 [SMILES: CN(C)c1ccc(NC(=O)N2CCC[C@@H](c3nc(-c4cccc(Cl)c4)c4ccccn34)C2)cc1] |
| 10-23 | 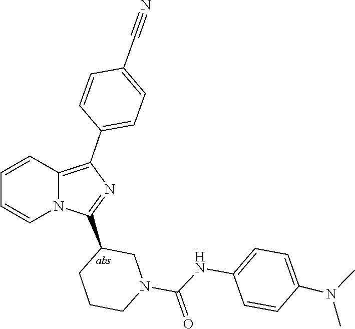 [SMILES: CN(C)c1ccc(NC(=O)N2CCC[C@@H](c3nc(-c4ccc(C#N)cc4)c4ccccn34)C2)cc1] |
| 10-24 | 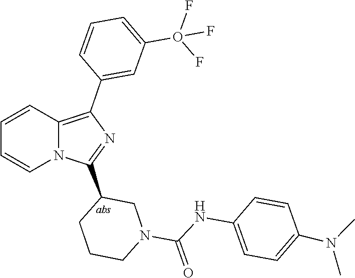 |
| 10-26 | 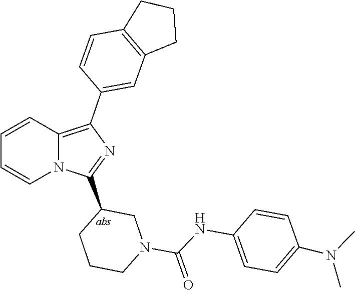 [SMILES: CN(C)c1ccc(NC(=O)N2CCC[C@@H](c3nc(-c4ccc5c(c4)CCC5)c4ccccn34)C2)cc1] |
| 10-27 | 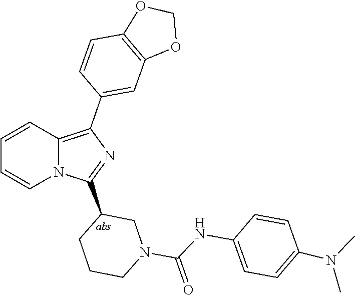 [SMILES: CN(C)c1ccc(NC(=O)N2CCC[C@@H](c3nc(-c4ccc5c(c4)OCO5)c4ccccn34)C2)cc1] |
| 10-28 | 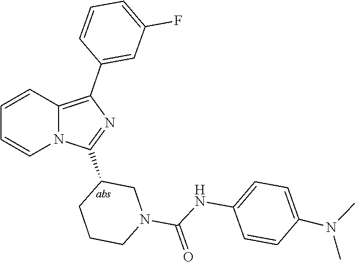 [SMILES: CN(C)c1ccc(NC(=O)N2CCC[C@H](c3nc(-c4cccc(F)c4)c4ccccn34)C2)cc1] |
| 10-29 | 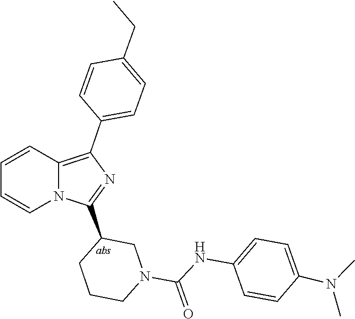 [SMILES: CCc1ccc(-c2nc([C@@H]3CCCN(C(=O)Nc4ccc(N(C)C)cc4)C3)n3ccccc23)cc1] |
| 10-30 | 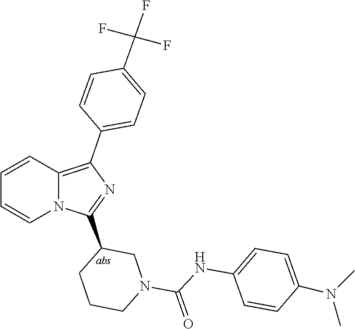 [SMILES: CN(C)c1ccc(NC(=O)N2CCC[C@@H](c3nc(-c4ccc(C(F)(F)F)cc4)c4ccccn34)C2)cc1] |
| 10-31 | 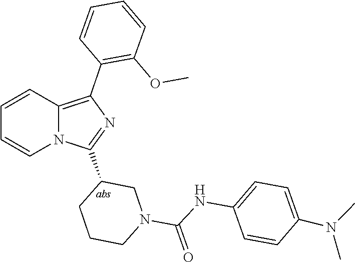 [SMILES: COc1ccccc1-c1nc([C@H]2CCCN(C(=O)Nc3ccc(N(C)C)cc3)C2)n2ccccc12] |
| 10-32 | 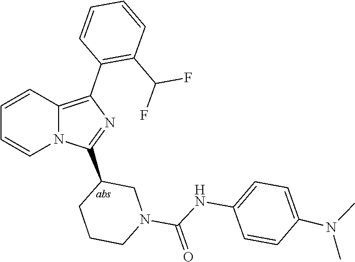 [SMILES: CN(C)c1ccc(NC(=O)N2CCC[C@@H](c3nc(-c4ccccc4C(F)F)c4ccccn34)C2)cc1] |
| 10-33 | 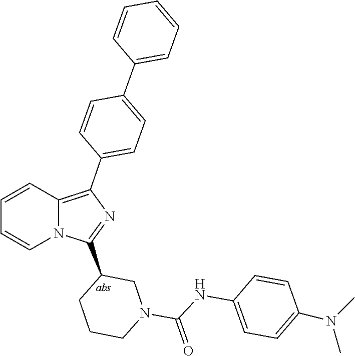 [SMILES: CN(C)c1ccc(NC(=O)N2CCC[C@@H](c3nc(-c4ccc(-c5ccccc5)cc4)c4ccccn34)C2)cc1] |
| 10-34 | 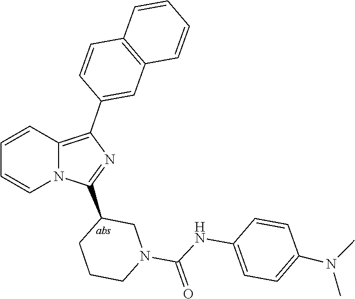 [SMILES: CN(C)c1ccc(NC(=O)N2CCC[C@@H](c3nc(-c4ccc5ccccc5c4)c4ccccn34)C2)cc1] |
| 10-35 | 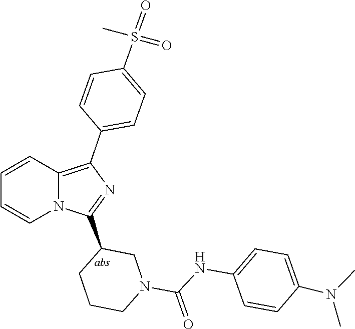 [SMILES: CN(C)c1ccc(NC(=O)N2CCC[C@@H](c3nc(-c4ccc(S(C)(=O)=O)cc4)c4ccccn34)C2)cc1] |
| 10-36 | 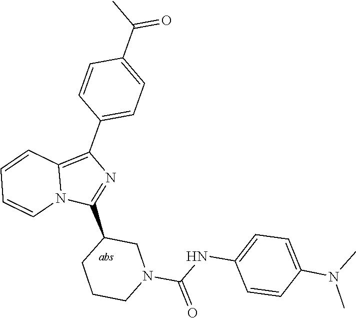 [SMILES: CC(=O)c1ccc(-c2nc([C@@H]3CCCN(C(=O)Nc4ccc(N(C)C)cc4)C3)n3ccccc23)cc1] |
| 10-37 | 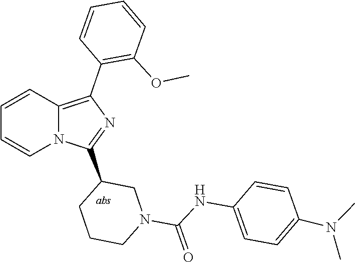 [SMILES: COc1ccccc1-c1nc([C@@H]2CCCN(C(=O)Nc3ccc(N(C)C)cc3)C2)n2ccccc12] |
| 10-40 | 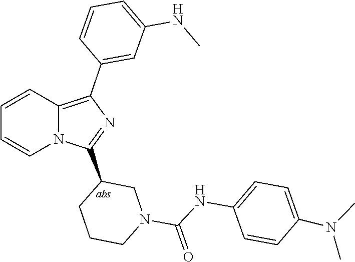 |
| 10-42 | 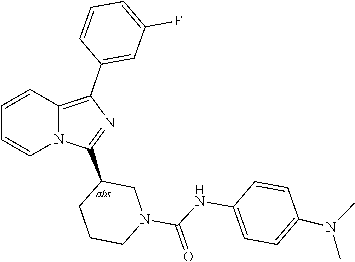 [SMILES: CN(C)c1ccc(NC(=O)N2CCC[C@@H](c3nc(-c4cccc(F)c4)c4ccccn34)C2)cc1] |
| 10-43 | 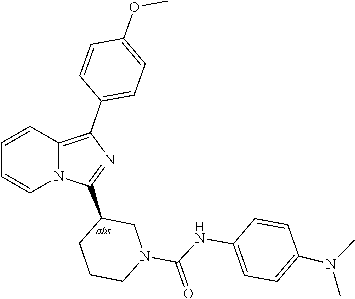 [SMILES: COc1ccc(-c2nc([C@@H]3CCCN(C(=O)Nc4ccc(N(C)C)cc4)C3)n3ccccc23)cc1] |
| 10-44 | 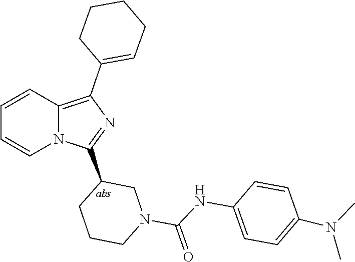 [SMILES: CN(C)c1ccc(NC(=O)N2CCC[C@@H](c3nc(C4=CCCCC4)c4ccccn34)C2)cc1] |
| 10-45 | 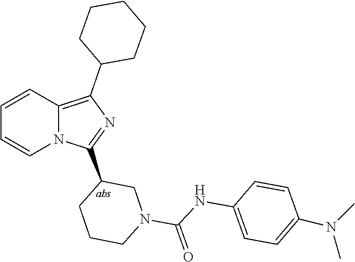 [SMILES: CN(C)c1ccc(NC(=O)N2CCC[C@@H](c3nc(C4CCCCC4)c4ccccn34)C2)cc1] |
| 10-46 | 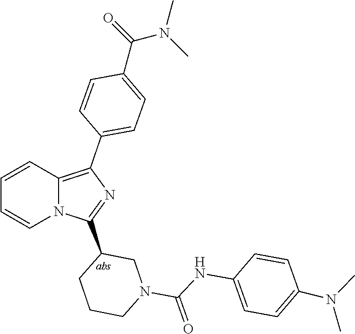 [SMILES: CN(C)C(=O)c1ccc(-c2nc([C@@H]3CCCN(C(=O)Nc4ccc(N(C)C)cc4)C3)n3ccccc23)cc1] |
| 10-47 | 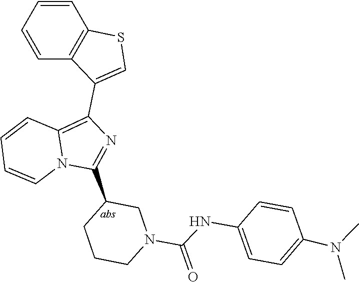 [SMILES: CN(C)c1ccc(NC(=O)N2CCC[C@@H](c3nc(-c4csc5ccccc45)c4ccccn34)C2)cc1] |
| 10-48 | 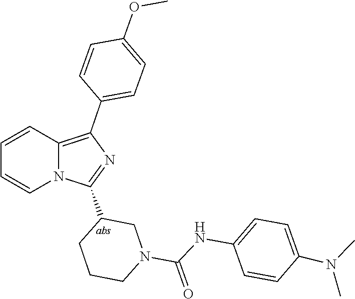 |
| 10-49 | 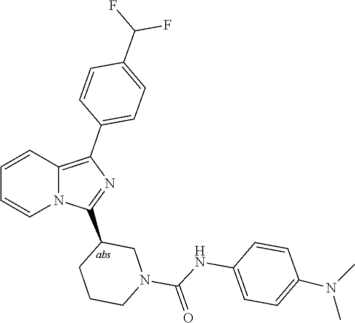 [SMILES: CN(C)c1ccc(NC(=O)N2CCC[C@@H](c3nc(-c4ccc(C(F)F)cc4)c4ccccn34)C2)cc1] |
| 10-50 | 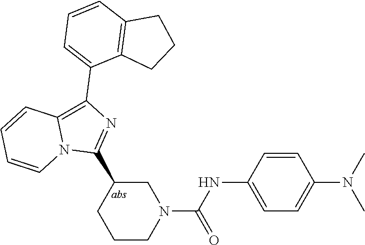 [SMILES: CN(C)c1ccc(NC(=O)N2CCC[C@@H](c3nc(-c4cccc5c4CCC5)c4ccccn34)C2)cc1] |
| 10-51 | 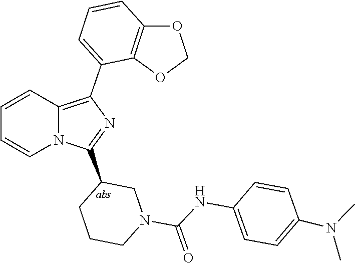 [SMILES: CN(C)c1ccc(NC(=O)N2CCC[C@@H](c3nc(-c4cccc5c4OCO5)c4ccccn34)C2)cc1] |
| 10-52 | 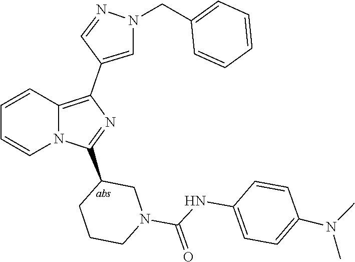 [SMILES: CN(C)c1ccc(NC(=O)N2CCC[C@@H](c3nc(-c4cnn(Cc5ccccc5)c4)c4ccccn34)C2)cc1] |
| 10-53 | 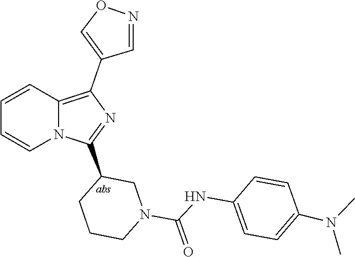 [SMILES: CN(C)c1ccc(NC(=O)N2CCC[C@@H](c3nc(-c4cnoc4)c4ccccn34)C2)cc1] |
| 10-54 | 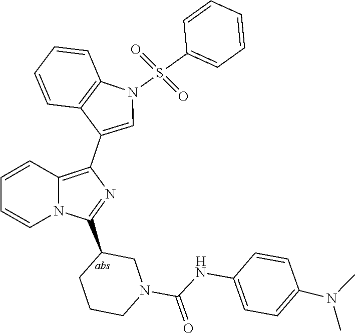 |
| 10-55 | 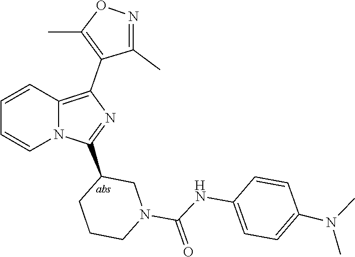 |
| 10-56 | 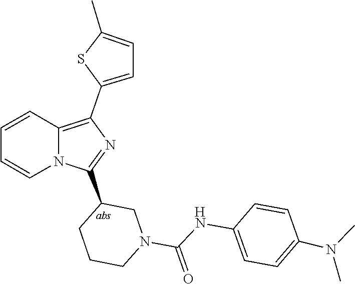 |
| 10-57 | 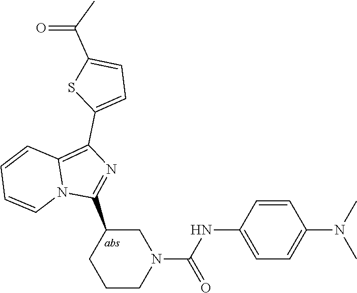 [SMILES: CC(=O)c1ccc(-c2nc([C@@H]3CCCN(C(=O)Nc4ccc(N(C)C)cc4)C3)n3ccccc23)s1] |
| 10-58 | 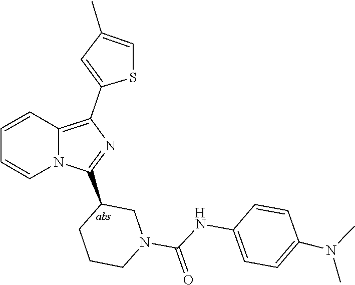 |
| 10-59 | 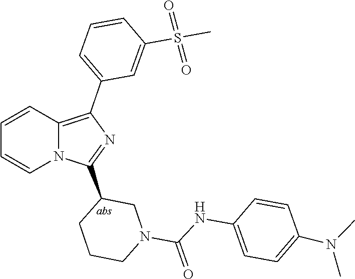 [SMILES: CN(C)c1ccc(NC(=O)N2CCC[C@@H](c3nc(-c4cccc(S(C)(=O)=O)c4)c4ccccn34)C2)cc1] |
| 10-60 | 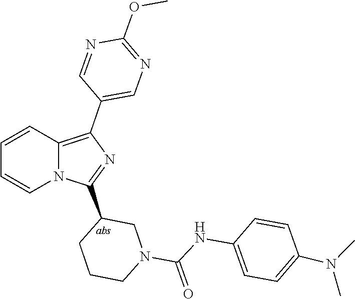 [SMILES: COc1ncc(-c2nc([C@@H]3CCCN(C(=O)Nc4ccc(N(C)C)cc4)C3)n3ccccc23)cn1] |
| 10-61 | 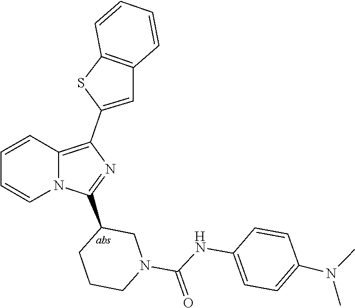 |
| 10-62 | 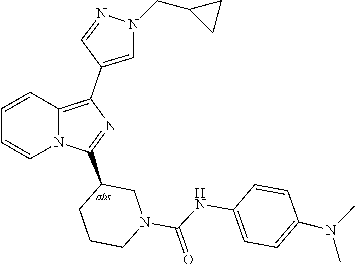 |
| 10-63 | 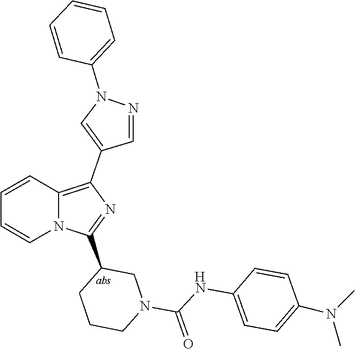 |
| 10-64 | 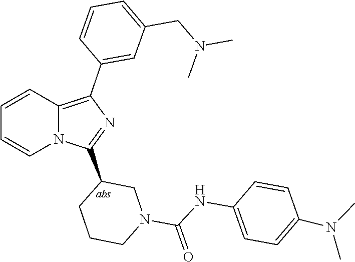 [SMILES: CN(C)Cc1cccc(-c2nc([C@@H]3CCCN(C(=O)Nc4ccc(N(C)C)cc4)C3)n3ccccc23)c1] |
| 10-65 | 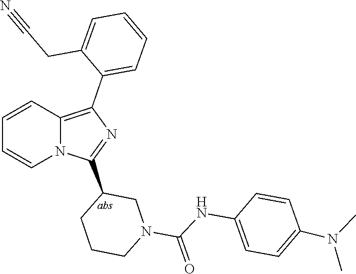 [SMILES: CN(C)c1ccc(NC(=O)N2CCC[C@@H](c3nc(-c4ccccc4CC#N)c4ccccn34)C2)cc1] |
| 10-66 | 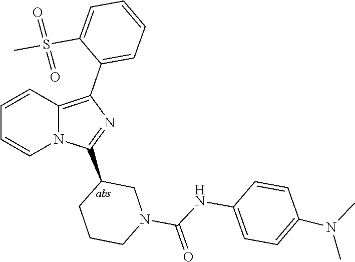 [SMILES: CN(C)c1ccc(NC(=O)N2CCC[C@@H](c3nc(-c4ccccc4S(C)(=O)=O)c4ccccn34)C2)cc1] |
| 10-67 | 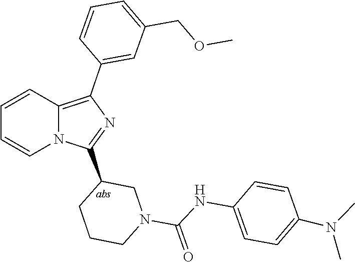 [SMILES: COCc1cccc(-c2nc([C@@H]3CCCN(C(=O)Nc4ccc(N(C)C)cc4)C3)n3ccccc23)c1] |
| 10-68 | 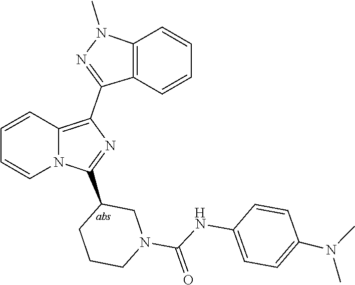 |
| 10-69 | 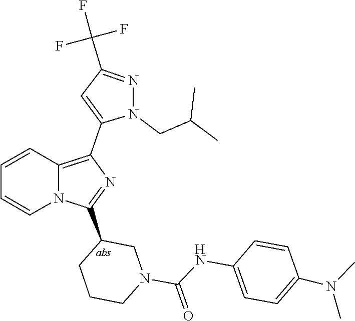 |
| 10-70 | 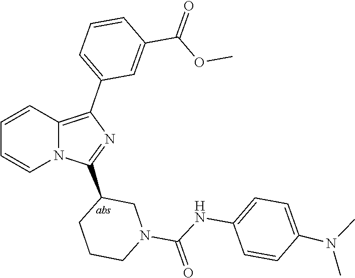 [SMILES: COC(=O)c1cccc(-c2nc([C@@H]3CCCN(C(=O)Nc4ccc(N(C)C)cc4)C3)n3ccccc23)c1] |
| 10-71 | 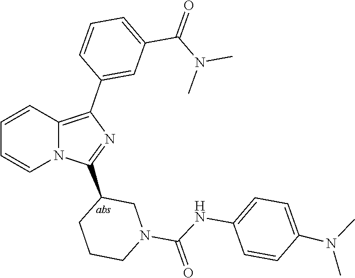 [SMILES: CN(C)C(=O)c1cccc(-c2nc([C@@H]3CCCN(C(=O)Nc4ccc(N(C)C)cc4)C3)n3ccccc23)c1] |
| 10-72 | 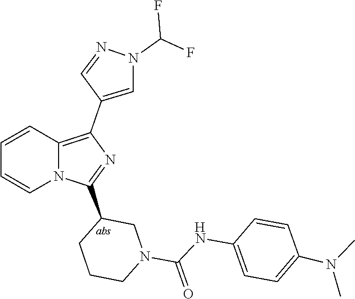 |
| 20-1 | 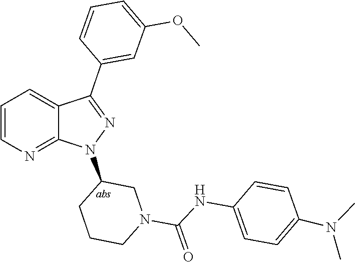 [SMILES: COc1cccc(-c2nn([C@@H]3CCCN(C(=O)Nc4ccc(N(C)C)cc4)C3)c3ncccc23)c1] |
| 20-2 | 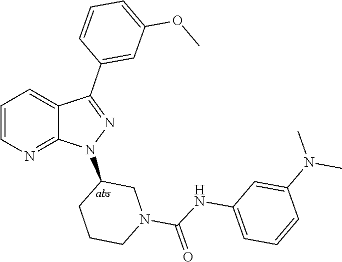 [SMILES: COc1cccc(-c2nn([C@@H]3CCCN(C(=O)Nc4cccc(N(C)C)c4)C3)c3ncccc23)c1] |
| 20-3 | 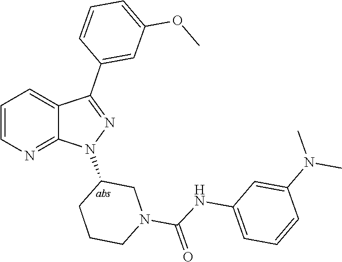 [SMILES: COc1cccc(-c2nn([C@H]3CCCN(C(=O)Nc4cccc(N(C)C)c4)C3)c3ncccc23)c1] |
| 20-4 | 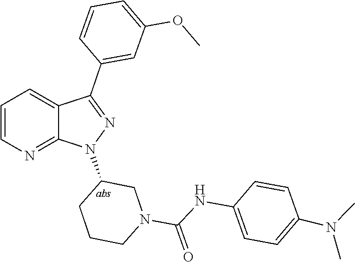 [SMILES: COc1cccc(-c2nn([C@H]3CCCN(C(=O)Nc4ccc(N(C)C)cc4)C3)c3ncccc23)c1] |
| 21-5 | 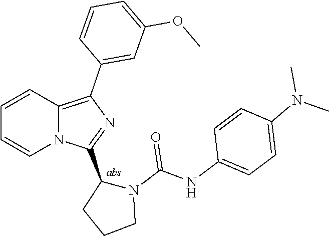 [SMILES: COc1cccc(-c2nc([C@@H]3CCCN3C(=O)Nc3ccc(N(C)C)cc3)n3ccccc23)c1] |
| 21-6 | 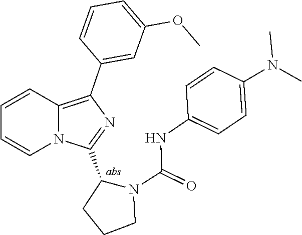 [SMILES: COc1cccc(-c2nc([C@H]3CCCN3C(=O)Nc3ccc(N(C)C)cc3)n3ccccc23)c1] |
| 23-1 | 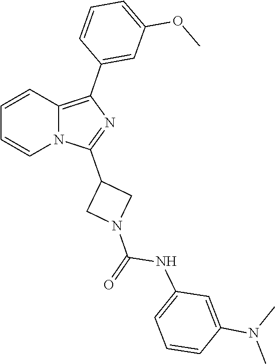 [SMILES: COc1cccc(-c2nc(C3CN(C(=O)Nc4cccc(N(C)C)c4)C3)n3ccccc23)c1] |
| 23-2 | 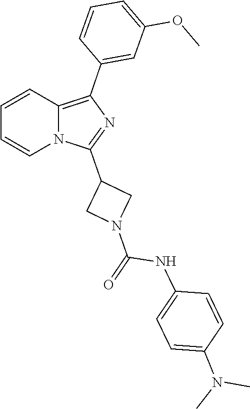 [SMILES: COc1cccc(-c2nc(C3CN(C(=O)Nc4ccc(N(C)C)cc4)C3)n3ccccc23)c1] |
| 24-1 | 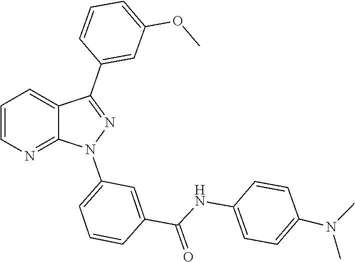 [SMILES: COc1cccc(-c2nn(-c3cccc(C(=O)Nc4ccc(N(C)C)cc4)c3)c3ncccc23)c1] |
| 24-2 | 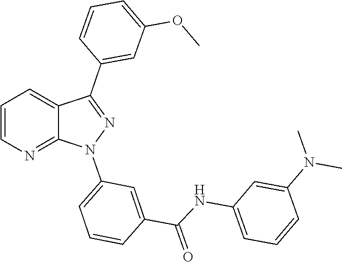 [SMILES: COc1cccc(-c2nn(-c3cccc(C(=O)Nc4cccc(N(C)C)c4)c3)c3ncccc23)c1] |
| 25-1 | 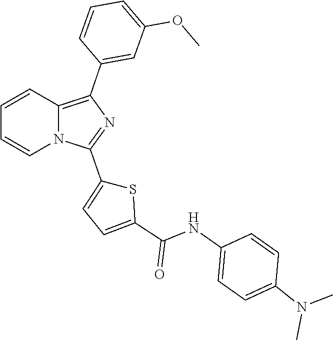 [SMILES: COc1cccc(-c2nc(-c3ccc(C(=O)Nc4ccc(N(C)C)cc4)s3)n3ccccc23)c1] |
| 26-1 | 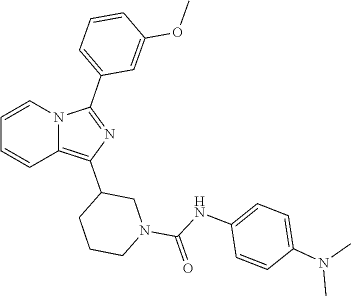 [SMILES: COc1cccc(-c2nc(C3CCCN(C(=O)Nc4ccc(N(C)C)cc4)C3)c3ccccn23)c1] |

or a pharmaceutically acceptable salt, isomer, hydrate, solvate, or isotope thereof.

62. The compound of claim 1, wherein the compound has the following structure (II): 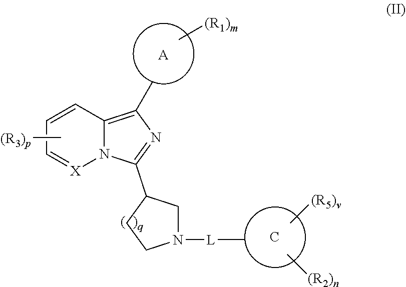
or a pharmaceutically acceptable salt, isomer, hydrate, solvate, or isotope thereof, wherein:
A is carbocyclyl or heterocyclyl, or A is 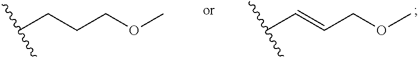
C is carbocyclyl or heterocyclyl;
L is a linker having the structure —C(O)—, —C(O)-alkyl-, —C(O)—NRa-, —C(O)—NRa-alkyl-, —S(O)₂—, or —S(O)₂-alkyl-;
X is CR₃, CH, or N;
R₁ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, alkoxycarbonyl, aminylcarbonyl, alkylsulfonyl, cycloalkylalkyl, aryl, aralkyl, or arylsulfonyl;
R₂ is hydroxyl, hydroxylalkyl, aminyl, aminylalkyl, cyanoalkyl, alkoxyalkyl, aminylcarbonyl, alkylsulfonyl, alkylsufonylalkyl, heterocyclyl, or alkylheteroaryl;
R₃ is alkyl or alkoxy;
R₅ is halo or alkyl;
Ra is H or alkyl;
m is 0, 1, or 2;
n is 1, 2, or 3;
p is 0, 1, 2, or 3;
q is 0, 1, or 2; and
v is 0 or 1.

63-117. (canceled)

118. The compound of claim 1, wherein the compound has the following structure (III): 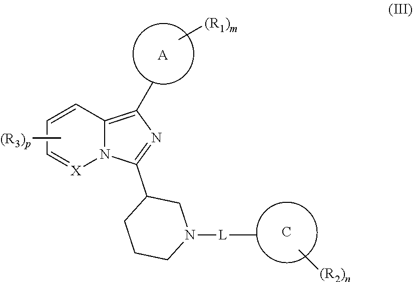
or a pharmaceutically acceptable salt, isomer, hydrate, solvate, or isotope thereof, wherein:
A is cycloalkyl or heterocyclyl;
C is carbocyclyl or heterocyclyl, or C is optionally absent when L is —C(O)—NRa- and is R₂ is alkyl, aminylalkyl, alkoxyalkyl, or alkylsufonylalkyl;
L is a linker having the structure —C(O)—, —C(O)-alkyl-, —C(O)—Nra-, —C(O)—Nra-alkyl-, —S(O)₂—, or —S(O)₂-alkyl-;
X is CR₃, CH, or N;
R₁ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, alkoxycarbonyl, aminylcarbonyl, alkylsulfonyl, cycloalkylalkyl, aryl, aralkyl, or arylsulfonyl;
R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
Ra is H or alkyl;
m is 0, 1, or 2;
n is 0, 1, 2, or 3; and
p is 0, 1, 2, or 3.

119-169. (canceled)

170. The compound of claim 1, wherein the compound has the following structure (IV): 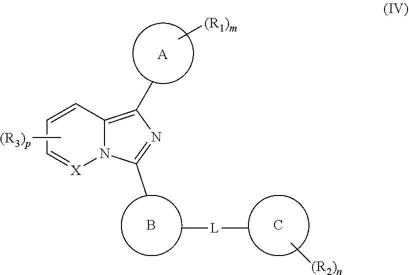
or a pharmaceutically acceptable salt, isomer, hydrate, solvate, or isotope thereof, wherein:
A is carbocyclyl or heterocyclyl;
B is heterocyclyl;
C is a nitrogen containing heterocyclyl;
L is a linker having the structure —C(O)—, —C(O)-alkyl-, —C(O)—Nra-, —C(O)—Nra-alkyl-, —S(O)₂—, or —S(O)₂-alkyl-;
X is CR₃, CH, or N;
R₁ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, alkoxycarbonyl, aminylcarbonyl, alkylsulfonyl, cycloalkylalkyl, aryl, aralkyl, or arylsulfonyl;
R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
Ra is H or alkyl;
m is 0, 1, or 2;
n is 0, 1, 2, or 3; and
p is 0, 1, 2, or 3.

171-219. (canceled)

220. The compound of claim 1, wherein the compound has the following structure (V): 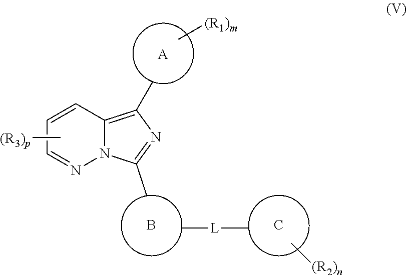
or a pharmaceutically acceptable salt, isomer, hydrate, solvate, or isotope thereof, wherein:
A and B are each individually carbocyclyl or heterocyclyl;
C is carbocyclyl or heterocyclyl, or C is optionally absent when L is —C(O)—Nra- and R₂ is alkyl, aminylalkyl, alkoxyalkyl, or alkylsufonylalkyl;
L is a linker having the structure —C(O)—, —C(O)-alkyl-, —C(O)—Nra-, —C(O)—Nra-alkyl-, —S(O)₂—, or —S(O)₂-alkyl-;
R₁ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, alkoxycarbonyl, aminylcarbonyl, alkylsulfonyl, cycloalkylalkyl, aryl, aralkyl, or arylsulfonyl;
R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
Ra is H or alkyl;
m is 0, 1, or 2;
n is 0, 1, 2, or 3; and
p is 0, 1, 2, or 3.

221-277. (canceled)

278. The compound of claim 1, wherein the compound has the following structure (VI): 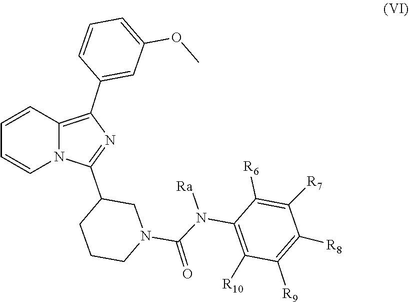
or a pharmaceutically acceptable salt, isomer, hydrate, solvate, or isotope thereof, wherein:
Ra is H or alkyl; and
either (1) R₆-R₁₀ are each individually H;
(2) R₆ and R₁₀ are H and R₇, R₈, and R₉ are each individually methoxy;
(3) R₆ is methyl and R₇ is methoxy and R₈-R₁₀ are each individually H;
(4) R₉ and R₁₀ are both H, and all but one of R₆-R₈ is H and the non-hydrogen substituent is selected from:
R₆ is F, Cl, methyl, ethyl, methoxy, ethoxy, OCF₃, —C(O)CH₃,
R₇ is Cl, cyano, ethyl, CF₃, OCF₃, ethoxy, —C(O)CH₃, and
R₈ is F, Cl, methyl, ethyl, methoxy, ethoxy, OCF₃, phenyl;
(5) R₆ and R₇ join together with the phenyl ring to which they are attached to form 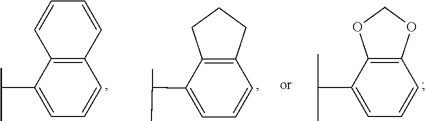
(6) R₇ and R₈ join together with the phenyl ring to which they are attached to form 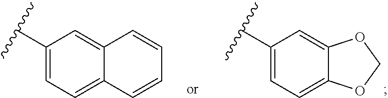 [SMILES: *c1ccc2ccccc2c1 ; *c1ccc2c(c1)OCO2]
or
(7) R₈ and R₉ join together with the phenyl ring to which they are attached to form

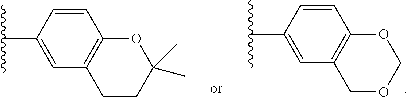 [SMILES: *c1ccc2c(c1)CCC(C)(C)O2 ; *c1ccc2c(c1)COCO2]

279-291. (canceled)

292. The compound of claim 1, wherein the compound has the following structure (VII): 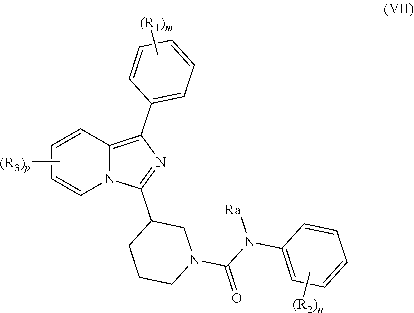
or a pharmaceutically acceptable salt, isomer, hydrate, solvate, or isotope thereof, wherein:
R₁ is hydroxyl, halo, cyano, alkyl, haloalkyl, hydroxylalkyl, C₂-C₆ alkoxy, haloalkoxy, aminyl, aminylalkyl, cyanoalkyl, alkoxyalkyl, alkylcarbonyl, alkoxycarbonyl, aminylcarbonyl, alkylsulfonyl, cycloalkylalkyl, aryl, aralkyl, or arylsulfonyl;
R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
Ra is H or alkyl;
m is 0, 1, or 2;
n is 0, 1, 2, or 3; and
p is 0, 1, 2, or 3.

293-316. (canceled)

317. The compound of claim 1, wherein the compound has the following structure (VIII): 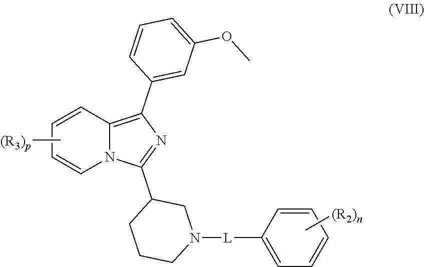
or a pharmaceutically acceptable salt, isomer, hydrate, solvate, or isotope thereof, wherein:
L is a linker having the structure —C(O)—, —C(O)-alkyl-, —C(O)—Nra-, —C(O)—Nra-alkyl-, —S(O)₂—, or —S(O)₂-alkyl-;
R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
Ra is H or alkyl;
n is 0, 1, 2, or 3; and
p is 1, 2, or 3.

318-339. (canceled)

340. The compound of claim 1, wherein the compound has the following structure (IX): 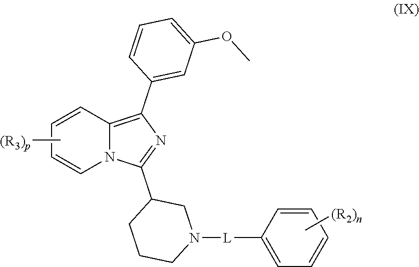
or a pharmaceutically acceptable salt, isomer, hydrate, solvate, or isotope thereof, wherein:
L is a linker having the structure —C(O)—, —C(O)-alkyl-, —S(O)₂—, or —S(O)₂-alkyl-;
each R₂ is individually hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl; and
R₃ is alkyl or alkoxy;
n is 0, 1, 2, or 3; and
p is 0, 1, 2, or 3.

341-359. (canceled)

360. The compound of claim 1, wherein the compound has the following structure (X): 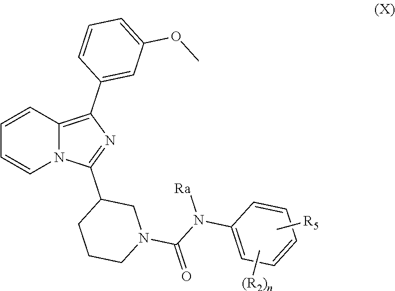
or a pharmaceutically acceptable salt, isomer, hydrate, solvate, or isotope thereof, wherein:
R₂ is hydroxyl, halo, cyano, alkyl, haloalkyl, hydroxylalkyl, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₅ is alkyl;
Ra is H or alkyl; and
n is 1, 2, or 3.

361-370. (canceled)

371. The compound of claim 1, wherein the compound has the following structure (XI): 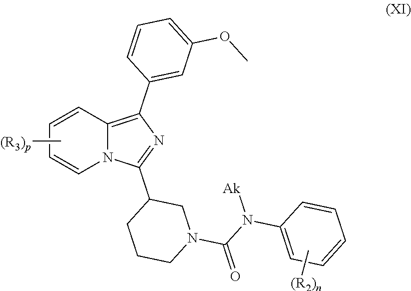
or a pharmaceutically acceptable salt, isomer, hydrate, solvate, or isotope thereof, wherein:
Ak is alkyl;
R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
n is 0, 1, 2, or 3; and
p is 0, 1, 2, or 3.

372-386. (canceled)

387. The compound of claim 1, wherein the compound has the following structure (XII): 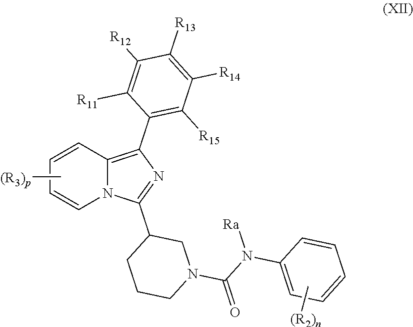
or a pharmaceutically acceptable salt, isomer, hydrate, solvate, or isotope thereof, wherein:
either (1) R₁₃ is methoxy and R₁₁, R₁₂, R₁₄, and R₁₅ are each individually H,
(2) R₁₁ is methoxy and R₁₂, R₁₃, R₁₄, and R₁₅ are each individually H,
(3) R₁₅ is methoxy and R₁₁, R₁₂, R₁₃, and R₁₄ are each individually H, or
(4) R₁₂ and R₁₄ are both methoxy and R₁₁, R₁₃, and R₁₅ are each individually H; and
R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
Ra is H or alkyl;
n is 0, 1, 2, or 3; and
p is 0, 1, 2, or 3.

388-408. (canceled)

409. The compound of claim 1, wherein the compound has the following structure (XIII): 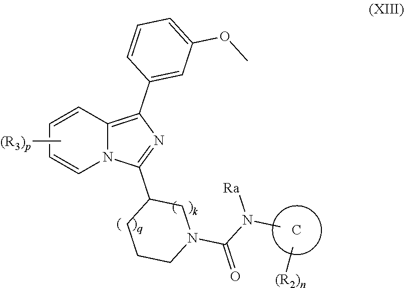
or a pharmaceutically acceptable salt, isomer, hydrate, solvate, or isotope thereof, wherein:
either (1) k is 0 and q is 1, or (2) k is 1 and q is 0;
C is phenyl, or C is methyl and n is 0;
R₂ is hydroxyl, halo, cyano, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
Ra is H or alkyl;
n is 0, 1, 2, or 3; and
p is 0, 1, 2, or 3.

410-430. (canceled)

431. The compound of claim 1, wherein the compound has the following structure (XIV): 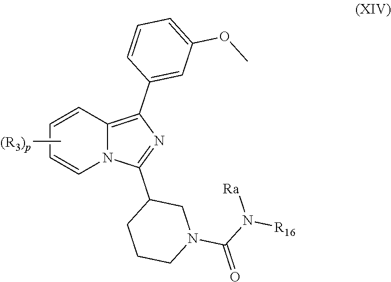
or a pharmaceutically acceptable salt, isomer, hydrate, solvate, or isotope thereof, wherein:
R₁₆ is methyl, alkoxyalkyl, alkylsufonylalkyl, C₃-C₅ cycloalkyl, C₃-C₅ cycloalkylC₁-C₆alkyl, alkylC₃-C₅cycloalkyl, benzyl, 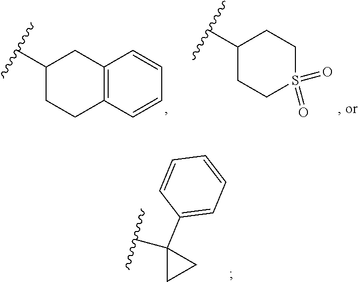 [SMILES: *C1CCc2ccccc2C1 ; *C1CCS(=O)(=O)CC1 ; *C1(c2ccccc2)CC1]
R₃ is alkyl or alkoxy; and
Ra is H or alkyl;
p is 0, 1, 2, or 3.

432-455. (canceled)

456. A method of treating a MRGPRD dependent condition by administering to a subject in need thereof an effective amount of a compound having the following structure (XV): 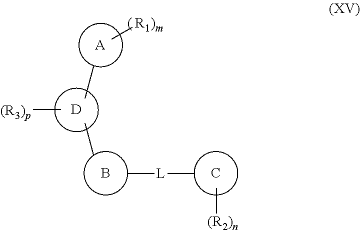
or a pharmaceutically acceptable salt, isomer, hydrate, solvate, or isotope thereof, wherein:
A is carbocyclyl, heterocyclyl, alkoxyalkyl, or alkoxyalkenyl;
B is phenyl, thiophenyl, piperidinyl, pyrrolidinyl, or azetidinyl;
C is carbocyclyl or heterocyclyl, or C is optionally absent when L is —C(O)—Nra- and R₂ is alkyl, aminylalkyl, alkoxyalkyl, or alkylsufonylalkyl;
D is 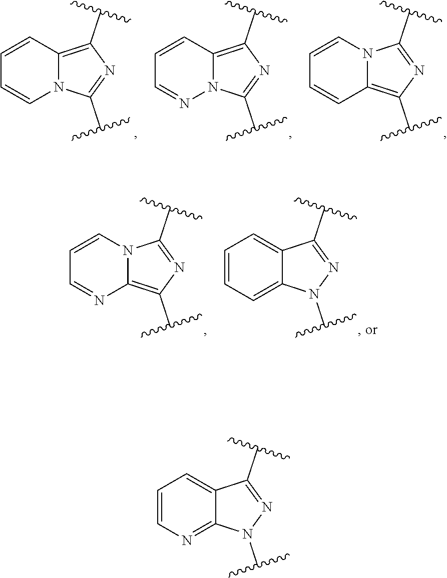 [SMILES: *c1nc(*)n2ccccc12 ; *c1nc(*)n2ncccc12 ; *c1nc(*)n2ccccc12 ; *c1nc(*)n2cccnc12 ; *c1nn(*)c2ccccc12 ; *c1nn(*)c2ncccc12]
L is a linker having the structure —C(O)—, —C(O)-alkyl-, —C(O)—Nra-, —C(O)—Nra-alkyl-, —S(O)₂—, or —S(O)₂-alkyl-;
R₁ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, alkoxycarbonyl, aminylcarbonyl, alkylsulfonyl, cycloalkylalkyl, aryl, aralkyl, or arylsulfonyl;
R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
Ra is H or alkyl;
m is 0, 1, or 2;
n is 0, 1, 2, or 3; and
p is 0, 1, 2, or 3.

457. The method of claim 456, wherein the method of treating a MRGPRD dependent condition by administering to a subject in need thereof an effective amount of a compound having the following structure (Xva): 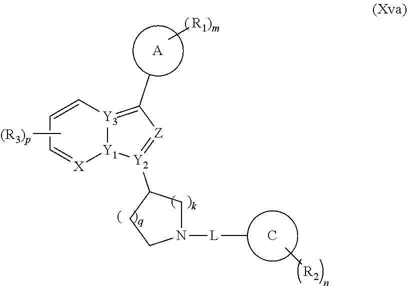
or a pharmaceutically acceptable salt, isomer, hydrate, solvate, or isotope thereof, wherein:
A is carbocyclyl, heterocyclyl, alkoxyalkyl, or alkoxyalkenyl;
C is carbocyclyl or heterocyclyl, or C is optionally absent when L is —C(O)—Nra- and R₂ is alkyl, aminylalkyl, alkoxyalkyl, or alkylsufonylalkyl;
X is CR₃, CH, or N;
Y₁, Y₂, and Y₃ are each independently C or N;
Z is CH or N;
L is a linker having the structure —C(O)—, —C(O)-alkyl-, —C(O)—Nra-, —C(O)—Nra-alkyl-, —S(O)₂—, or —S(O)₂-alkyl-;
R₁ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, alkoxycarbonyl, aminylcarbonyl, alkylsulfonyl, cycloalkylalkyl, aryl, aralkyl, or arylsulfonyl;
R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
Ra is H or alkyl;
m is 0, 1, or 2;
n is 0, 1, 2, or 3;
p is 0, 1, 2, or 3;
q is 0, 1, or 2; and
k is 0, 1, or 2.

458-465. (canceled)

466. The method of claim 456, wherein the method of treating a MRGPRD dependent condition by administering to a subject in need thereof an effective amount of a compound having the following structure (XVb): 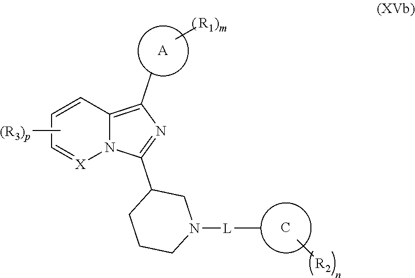
or a pharmaceutically acceptable salt, isomer, hydrate, solvate, or isotope thereof, wherein:
A is carbocyclyl, heterocyclyl, alkoxyalkyl, or alkoxyalkenyl;
C is carbocyclyl or heterocyclyl, or C is optionally absent when L is —C(O)—Nra- and R₂ is alkyl, aminylalkyl, alkoxyalkyl, or alkylsufonylalkyl;
X is CR₃, CH, or N;
L is a linker having the structure —C(O)—, —C(O)-alkyl-, —C(O)—Nra-, —C(O)—Nra-alkyl-, —S(O)₂—, or —S(O)₂-alkyl-;
R₁ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, alkoxycarbonyl, aminylcarbonyl, alkylsulfonyl, cycloalkylalkyl, aryl, aralkyl, or arylsulfonyl;
R₂ is hydroxyl, halo, cyano, alkyl, haloalkyl, hydroxylalkyl, alkoxy, haloalkoxy, aminyl, aminylalkyl, cyanoalkyl, alkoxyalkyl, alkylcarbonyl, aminylcarbonyl, alkylsulfonyl, alkylsufonylalkyl, aryl, heterocyclyl, or alkylheteroaryl;
R₃ is alkyl or alkoxy;
Ra is H or alkyl;
m is 0, 1, or 2;
n is 0, 1, 2, or 3; and
p is 0, 1, 2, or 3.

467-530. (canceled)

531. A pharmaceutical composition comprising a compound of claim 1 or a pharmaceutically acceptable salt, isomer, hydrate, solvate or isotope thereof and at least one pharmaceutically acceptable excipient.

532. A method of modulating a Mas-Related G-Protein Receptor (MRGPR) D by contacting MRGPRD with an effective amount of a compound of claim 1.

533. A method of treating a MRGPRD dependent condition by administering to a subject in need thereof an effective amount of a compound of claim 1.

534. The method of claim 533, wherein the MRGPRD dependent condition is a pain associated condition, an itch associated condition, an ocular associated condition, a cardiovascular and renal disease associate condition, an inflammatory or autoimmune disorder, o a cognitive impairment associated condition, or a cancer related condition.

535-547. (canceled)
